(19) United States
(12) Patent Application Publication
(10) Pub. No.: US 2022/0244263 A1
(43) Pub. Date: Aug. 4, 2022

(54) METHODS FOR TREATING SMALL CELL NEUROENDOCRINE AND RELATED CANCERS

(71) Applicant: The Regents of the University of California, 12th Floor, CA (US)

(72) Inventors: Thomas G. GRAEBER, Los Angeles, CA (US); Nikolas G. BALANIS, Los Angeles, CA (US); Katherine M. SHEU, Los Angeles, CA (US); Owen N. WITTE, Los Angeles, CA (US); Favour ESEDEBE, Los Angeles, CA (US); Jung Wook PARK, Los Angeles, CA (US)

(73) Assignee: The Regents of the University of California, Oakland, CA (US)

(21) Appl. No.: 17/613,881

(22) PCT Filed: May 28, 2020
(86) PCT No.: PCT/US2020/034954

§ 371 Date: Nov. 23, 2021

Related U.S. Application Data
(60) Provisional application No. 62/853,551, filed on May 28, 2019.

(51) Int. Cl. G01N 33/574; A61K 45/06

(52) U.S. Cl. CPC G01N 33/57484; A61K 45/06

(57) ABSTRACT

The current disclosure provides for methods of identifying and treating small cell neuroendocrine (SCN) tumors and small-round-blue cell tumor (SRBCT). Aspects of the disclosure relate to a method comprising measuring the level of expression of one or more biomarkers from Tables 1-3 in a biological sample from a cancer patient. Further aspects relate to a method for treating small cell neuroendocrine (SCN) cancer or small-round-blue cell tumor (SRBCT) in a patient comprising administering a cancer treatment to the patient, wherein the cancer treatment is a treatment selected from Table 4A, 4B, or combinations of treatments from Table 4A and/or 4B.

This application claims priority to U.S. Provisional Patent Application Ser. No. 62/853,551 filed May 28, 2019, which are incorporated by reference herein in their entirety.

This invention was made with government support under Grant Numbers CA092131, CA222877, GM008042, awarded by the National Institutes of Health. The government has certain rights in the invention.

### BACKGROUND OF THE INVENTION

### Field of the Invention

This invention relates to the field of medicine.

### Background

Small cell neuroendocrine cancers (SCNCs) are a highly aggressive cancer subtype that has been observed to arise in multiple tissues, more commonly reported in lung, with rare cases in prostate, bladder, breast, skin, gastrointestinal tract, and cervix. SCNCs that arise from different tissues share characteristic morphology- and marker-based histology such as high nuclear to cytoplasm ratios, frequent mitotic figures, and granular chromatin. At the molecular level, TP53 and RB1 loss and/or inactivating mutations are essentially obligatory for SCNCs of the lung and highly enriched in SCNC's of the prostate. Additionally, SCNCs from both tissues share common neuroendocrine markers such as chromogranin A (CHGA) and synaptophysin (SYP).

Neuroendocrine cells are found in numerous tissues, but the cell of origin of SCNCs across tissues is unclear. Small cell lung cancer (SCLC) can arise de novo, possibly from a cell of neuroendocrine origin, as seen in mouse models or in transformation experiments performed in distinct lung cell types. A second proposed mechanism for SCLC origin is through transdifferentiation from another non-neuroendocrine cell lineage, which has been observed in patient tumors, and studied in mouse in vivo experiments.

De novo neuroendocrine prostate cancers (NEPC) are rare (approximately 1% of cases), but exhibit a similarly aggressive phenotype. As in lung cancer, evidence for transdifferentiation from a prostate adenocarcinoma to a small cell neuroendocrine (SCN) state in response to androgen suppression therapy has been reported, with 25-40% of resulting resistant tumors expressing neuroendocrine markers. Thus, across multiple tissues types, adenocarcinomas have been observed to escape targeted therapy through evolution to a histologically similar SCN phenotype, highlighting the need to characterize and develop therapies for this aggressive cancer outcome.

There are currently no effective therapies for SCNCs. SCNC is considered a systemic disease and typically leads to early metastases. Etoposide or platinum-based chemotherapies are the primary first-line treatment modalities. These treatments are only transiently effective, and 5-year survival rates for SCN lung and prostate cancers are less than 20%. In the progression to treatment-resistant SCN cancers, roles for shared oncogenic transcription factors such as MYCN, and epigenetic regulators such as EZH2, have been described. There is a need in the art for more effective detection and treatment methods for SCN cancers.

### SUMMARY OF THE INVENTION

The current disclosure provides for methods of identifying and/or treating small cell neuroendocrine (SCN) tumors and small-round-blue cell tumor (SRBCT). Aspects of the disclosure relate to a method comprising measuring the level of expression of one or more biomarkers from Tables 1-3 in a biological sample from a cancer patient. Aspects of the disclosure relate to a method comprising measuring the level of expression of one or more biomarkers from Table 1 in a biological sample from a cancer patient. Aspects of the disclosure relate to a method comprising measuring the level of expression of one or more biomarkers from Table 2 in a biological sample from a cancer patient. Aspects of the disclosure relate to a method comprising measuring the level of expression of one or more biomarkers from Table 3 in a biological sample from a cancer patient.

Further aspects relate to a method for treating small cell neuroendocrine (SCN) cancer or small-round-blue cell tumor (SRBCT) in a patient comprising administering a cancer treatment to the patient, wherein the cancer treatment is a treatment selected from Table 4A or 4B, or combinations of treatments from Table 4A and/or 4B.

Further aspects relate to a method for treating small cell neuroendocrine (SCN) cancer or small-round-blue cell tumor (SRBCT) in a patient comprising administering a cancer treatment to the patient, wherein the cancer treatment is a targeted treatment that binds to and inhibits or activates a target, wherein the target is one listed in Table 5A or 5B, or combinations of targets listed in Table 5A and/or 5B.

Further aspects relate to a method for treating SCN cancer or SRBCT in a patient comprising administering a cancer treatment to a patient determined to have a SCN cancer or SRBCT, wherein the patient was determined to have a SCN or SRBCT by measuring the expression level of one or more biomarkers from tables 1-3 in a biological sample from the patient.

Further aspects relate to a method for prognosing a cancer patient or for diagnosing a SCN or SRBCT cancer, comprising: measuring the expression level of one or more biomarkers from Tables 1-3 in a biological sample from the patient; comparing the expression level of the at least one biomarker to a control level of expression, wherein the control level of expression comprises the level of expression of the biomarkers in a SCN or SRBCT cancer; and diagnosing the patient as having a SCN or SRBCT cancer when the level of expression of the measured biomarker is not substantially different from the control level of expression.

Further aspects of the disclosure relate to a method for treating SCN or SRBCT cancer in a patient comprising administering a cancer treatment to a patient determined to have a SCN or SRBCT cancer, wherein the patient was determined to have a SCN or SRBCT cancer by measuring the expression level of at least ten biomarkers from tables 1-3 in a biological sample from the patient; wherein the biomarker has an absolute value of the signature weight of greater than 0.025; and wherein the treatment comprises ABT-263, NSC-207895, NPK76-II-72-1, XMD13-2, MP470, BX-912, GW-2580, GSK1070916, WZ3105, GSK690693, OSI-027, FK866, I-BET-762, GSK429286A, UNC0638, PHA-793887, QL-X-138, Vorinostat, Tubastatin A, CX-5461, or combinations thereof.

Further aspects relate to a method for treating SCN or SRBCT cancer in a patient comprising administering a cancer treatment to a patient determined to have a SCN or SRBCT cancer, wherein the patient was determined to have a SCN or SRBCTcancer by measuring the expression level of at least ten biomarkers from tables 1-3 in a biological sample from the patient; wherein the biomarker has an absolute value of the signature weight of greater than 0.025; and wherein the treatment comprises vorinostat, VU0238429, CUDC-101, BX-912, oxaliplatin, alisertib, salinomycin, BMS-754807, KX2-391, D-64131, oxyquinoline, axitinib, barasertib, BI-78D3, M-344, evodiamine, UNBS-5162, vinorelbine, albendazole, BI-2536, or combinations thereof.

In some embodiments, at least one biomarker has an absolute value of the signature weight of greater than 0.04. In some embodiments, at least 2 biomarkers have an absolute value of the signature weight of greater than 0.03.

In some embodiments, the patient has been determined to have and/or diagnosed with a cancer. In some embodiments, the cancer comprises a cancer disclosed herein. In some embodiments, the cancer comprises epithelial cancer, SRBCT, brain cancer, melanoma, or a germ cell cancer. In some embodiments, the cancer is a hematological cancer. In some embodiments, hematological cancers are excluded. In some embodiments, the cancer comprises an epithelial cancer selected from adrenocortical carcinoma, bladder urothelial carcinoma, breast invasive carcinoma, cervical squamous cell carcinoma, endocervical adenocarcinoma, cholangiocarcinoma, colon adenocarcinoma, rectum adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, ovarian serous cystadenocarcinoma, pancreatic adenocarcinoma, prostate adenocarcinoma, stomach adenocarcinoma, thyroid carcinoma, thymoma, uterine corpus endometrial carcinoma, and uterine carcinosarcoma. In some embodiments, the cancer comprises an SRBCT cancer selected from sarcoma, Wilms Tumor (pediatric), Ewing's Sarcoma, medulloblastoma, neuroblastoma, rhabdomyosarcoma, and neuroblastoma (pediatric). In some embodiments, the cancer comprises a brain cancer selected from glioblastoma multiforme, brain lower grade glioma, mesothelioma, neuroblastoma (pediatric), pheochromocytoma and paraganglioma. In some embodiments, the cancer comprises a skin cancer. In some embodiments, the cancer comprises melanoma. In some embodiments, the cancer comprises skin cutaneous melanoma or uveal melanoma. In some embodiments, the cancer comprises testicular germ cell cancers. In some embodiments, the cancer comprises a hematological or hematopoietic cancer such as acute lymphoblastic leukemia (pediatric), acute myeloid leukemia (pediatric), acute myeloid leukemia induction fraction (pediatric), lymphoid neoplasm diffuse large B-cell lymphoma, and acute myeloid leukemia. In some embodiments, one or more of acute lymphoblastic leukemia (pediatric), acute myeloid leukemia (pediatric), acute myeloid leukemia induction fraction (pediatric), lymphoid neoplasm diffuse large B-cell lymphoma, and acute myeloid leukemia is excluded from the methods of the disclosure. In some embodiments, the cancer comprises a SCN cancer that is further defined as a cancer recited above or herein. In some embodiments, the cancer comprises small cell lung cancer. In some embodiments, the cancer excludes small cell lung cancer.

In some embodiments, the method comprises measuring the level of expression of at least five biomarkers from Tables 1-3. In some embodiments, the method comprises measuring the level of expression of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1500, 2000, 2500, 3000, 3500, 4000, 4500, 5000, 5000, 7000, 8000, 9000, 10000, 11000, 12000, 13000, 14000, 15000, 16000, 17000, or 18000 biomarkers (or any range derivable therein) from Tables 1-3.

In some embodiments, at least 2 of the measured biomarkers has an absolute value of the signature weight of greater than 0.025. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) of the measured biomarkers has an absolute value of the signature weight of greater than 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, or 0.05 (or any derivable range therein).

In some embodiments, the method comprises measuring the level of expression of at least 10 biomarkers from Tables 1-3 and wherein at least 5 of the measured biomarkers has an absolute value of the signature weight of greater than 0.025. In some embodiments, the method comprises measuring the level of expression of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 (or any derivable range thereof) biomarkers from Tables 1-3 and wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 (or any derivable range thereof) of the measured biomarkers has an absolute value of the signature weight of greater than 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, or 0.05 (or any derivable range therein). In some embodiments, the method comprises measuring the level of expression of at least 10 biomarkers from Tables 1-3 and wherein at least 5 of the measured biomarkers has an absolute value of the signature weight of greater than 0.03. In some embodiments, at least or at most 20, 30, 40, or 50 biomarkers are measured.

In some embodiments, the expression level of the measured biomarkers are/were determined to be not significantly different as compared to a control level of expression, wherein the control level of expression comprises the level of expression of the biomarkers in a SCN or SRBCT cancer. In some embodiments, the expression level of the measured biomarkers are/were determined to be significantly different as compared to a control level of expression, wherein the control level of expression comprises the level of expression of the biomarkers in a SCN or SRBCT cancer. In some embodiments, the expression level of the measured biomarkers are/were determined to be not significantly different as compared to a control level of expression, wherein the control level of expression comprises the level of expression of the biomarkers in a cancer that is not a SCN or SRBCT cancer. In some embodiments, the expression level of the measured biomarkers are/were determined to be significantly different as compared to a control level of expression, wherein the control level of expression comprises the level of expression of the biomarkers in a cancer that is not a SCN or SRBCT cancer. In some embodiments, the expression level of the measured biomarkers are/were determined to be not significantly different as compared to a control level of expression, wherein the control level of expression comprises the level of expression of the biomarkers in a non-cancerous sample. In some embodiments, the expression level of the measured biomarkers are/were determined to be significantly different as compared to a control level of expression, wherein the control level of expression comprises the level of expression of the biomarkers in a non-cancerous sample. In some embodiments, the expression level of the measured biomarkers are/were determined to be not significantly different as compared to a control level of expression, wherein the control level of expression comprises the level of expression of the biomarkers in a cancerous sample, wherein the cancer comprises a cancer described herein. In some embodiments, the expression level of the measured biomarkers are/were determined to be significantly different as compared to a control level of expression, wherein the control level of expression comprises the level of expression of the biomarkers in a cancerous sample, wherein the cancer comprises a cancer described herein.

In some embodiments, the one or more biomarkers has an absolute value of the signature weight of greater than 0.025. The term “absolute value” refers to the magnitude of a real number without regard to its sign. Signature weights are listed in Tables 1-3. In some embodiments, the one or more biomarkers has an absolute value of the signature weight of greater than or less than 0.000001, 0.000002, 0.000003, 0.000004, 0.000005, 0.000006, 0.000007, 0.000008, 0.000009, 0.00001, 0.000015, 0.00002, 0.000025, 0.00003, 0.000035, 0.00004, 0.000045, 0.00005, 0.000055, 0.00006, 0.000065, 0.00007, 0.000075, 0.00008, 0.000085, 0.00009, 0.000095, 0.0001, 0.00015, 0.0002, 0.00025, 0.0003, 0.00035, 0.0004, 0.00045, 0.0005, 0.00055, 0.0006, 0.00065, 0.0007, 0.00075, 0.0008, 0.00085, 0.0009, 0.00095, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, or 0.05 (or any range derivable therein).

In some embodiments, 1, 2, 3, 4, 5, 6, 7, or 8 biomarkers from Tables 1-3 are excluded from being measured for expression levels in the biological sample from the cancer patient. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1500, 2000, 2500, 3000, 3500, 4000, 4500, 5000, 5000, 7000, 8000, 9000, 10000, 11000, 12000, 13000, 14000, 15000, 16000, 17000, or 18000 biomarkers (or any range derivable therein) from Tables 1-3 are excluded from being measured for expression levels in the biological sample from the cancer patient.

In some embodiments, the method further comprises comparing the level of expression to the level of expression of the biomarker(s) in a control sample. In some embodiments, the control comprises a biological sample from a patient having a SCN or SRBCT cancer. In some embodiments, the control comprises a biological sample from a patient having a cancer disclosed herein. In some embodiments, the control comprises a biological sample from a patient having a cancer that is not a SCN cancer. In some embodiments, the control comprises a biological sample from a patient not having cancer.

In some embodiments, the method further comprises comprising comparing the level of expression to a control level of expression of the biomarker(s). In some embodiments, the control level of expression comprises the level of expression of the biomarker in a SCN or SRBCT cancerous sample. In some embodiments, the control level of expression comprises the level of expression of the biomarker in a cancerous sample from a cancer disclosed herein. In some embodiments, the control level of expression comprises the level of expression of the biomarker in cancerous sample that is not a SCN cancer. In some embodiments, the control level of expression comprises the level of expression of the biomarker in a non-cancerous sample.

In some embodiments, the biological sample comprises a tissue sample, a blood sample, a biopsy sample, a saliva sample, or a tumor sample. In some embodiments, the biological sample comprises a biological sample described herein. In some embodiments, the biological sample comprises tumor tissue. In some embodiments, the biological sample comprises metastatic tumor tissue or is from the lymph nodes.

In some embodiments, the subject has been treated for a cancer. In some embodiments, the treatment comprises a targeted therapy. In some embodiments, the method comprises or further comprises evaluating tumor size and/or lymph node status. In some embodiments, the method comprises or further comprises pathological and/or histological evaluation of a biological sample from the patient. In some embodiments, the sample is evaluated histologically for a SCN or SRBCT cell type. In some embodiments, the sample is determined to not have a SCN or SRBCT cancer after a histological analysis. In some embodiments, the method further comprises calculating a risk score for the patient. In some embodiments, the risk score indicates a risk of decreased overall survival, metastasis, and/or recurrence. In some embodiments, the risk score indicates the risk for having a SCN or SBRCT cancer.

In some embodiments, the method comprises or further comprises treating the patient for a SCN or SRBCT cancer. In some embodiments, the treatment inhibits a Target listed in Table 5A or 5B. In some embodiments, the treatment activates a target listed in Table 5A or 5B. In some embodiments, one or more of the targets in Table 5A or 5B is excluded from the methods of the disclosure. In some embodiments, the treatment comprises ABT-263, NSC-207895, NPK76-II-72-1, XMD13-2, MP470, BX-912, GW-2580, GSK1070916, WZ3105, GSK690693, OSI-027, FK866, I-BET-762, GSK429286A, UNC0638, PHA-793887, QL-X-138, Vorinostat, Tubastatin A, CX-5461, or combinations thereof. In some embodiments, the treatment comprises vorinostat, VU0238429, CUDC-101, BX-912, oxaliplatin, alisertib, salinomycin, BMS-754807, KX2-391, D-64131, oxyquinoline, axitinib, barasertib, BI-78D3, M-344, evodiamine, UNBS-5162, vinorelbine, albendazole, BI-2536, or combinations thereof. In some embodiments, the treatment comprises a drug listed in Table 4A, 4B, or combinations thereof. In some embodiments, the treatment excludes at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, or 256 (or any derivable range therein) drugs listed in Table 4A or 4B. In some embodiments, the treatment is a treatment in Table 4A or 4B with a sensitivity in SCN, SRBCT or Blood value of 1 or potential.

In some embodiments, the patient has been determined to have a SCN or SRBCT based on the level of expression of one or more biomarkers from tables 1-3 in a biological sample from the patient.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. It is contemplated that embodiments described in the context of the term “comprising” may also be implemented in the context of the term “consisting of” or “consisting essentially of.”

It is contemplated that any embodiment discussed in this specification can be implemented with respect to any method or composition provided herein, and vice versa. Furthermore, compositions described herein can be used to achieve methods described herein. It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-F. Pan-cancer convergence of small cell neuroendocrine carcinoma. A) Varimax-rotated PCA of adjacent normal, adenocarcinoma, and SCNC for lung and prostate. Ellipses represent 80% confidence regions. B) Varimax-rotated PCA of samples in panel A and bladder patient data. TCGA BLCA includes 4 SCN samples that were labeled separately (BLCA.SCN). PC1 and PC2 are reversed to show the SCN signature on the x-axis as in FIG. 1A. C) Gene loadings and selected top SCN-related genes of the varimax-rotated first principal component of the PCA (PCV1) of FIG. 1A. Enrichment analysis (GSEA) was run on this ranked gene list. Shown along the bottom are the top gene sets in the ‘neuro’ (red) and ‘immune’ (blue) categories, also shown in panel D. D) Top 10 gene sets from ‘neuro’ and ‘immune’ gene set categories. Lines mark false discovery rate (FDR) q-value of 0.05. E) Distribution of gene sets ranked by normalized enrichment score (NES). 5873 total genesets from C5 MSigDB collection. All listed categories enrichments are nominally significant (p value<0.001) by Kolmogorov-Smirnov test (p values located in FIG. 8D). Dashed lines mark FDR q-value<0.05 for individual gene sets in each direction. F) Left: Average rank ordering of VIPER activities across the three tissue types. Right: Zoom in to top of rank based inferred activity in each cancer separately. Combined p value across the three tissues by Stouffer's method (See also FIG. 8).

FIG. 2A-D. An epigenetic basis for the shared small cell neuroendocrine gene expression signature. A) RRHO heat map of lung and bladder SCN signatures defined by PLSR loadings. Number shown is the maximum −log 10(p value) of the RRHO heatmap. B) Methylation sites that define the lung or bladder SCN versus non-SCN dichotomy are enriched in open-sea regions rather than CG islands (random sample of 100K sites shown for visibility, distributions presented are representative of all sites). X-axis is the rank distance of the CG site from the nearest TSS (arrows point in direction of increasing distance). Y-axis is the rank value of individually run lung or bladder PLSR component 1 loadings, thus extreme values represent sites with differential methylation between SCN and non-SCN tumors. Waterfall plots show the relative values of the ranked loadings. C) Partial least squares regression (PLSR) analysis of DNA methylation data from patient bladder SCN and non-SCN tumor biopsy samples, and projection of lung LUAD and SCLC tumor samples onto this framework, or vice versa. PLSR component 1 is z-scored by tissue type. Dashed red lines separate training data (above line) from testing data (below line). D) Top 5 enrichment analysis (GSEA) terms for genes hypomethylated in SCN. Genes were ranked by averaged lung, prostate, bladder PLSR component 1 loadings. Dashed green line is at FDR p value=0.05. (See also FIGS. 9-10).

FIG. 3A-I. Pan-cancer identification of primary tumors with an SCN signature. A) Gene expression-based prediction of SCN phenotype in epithelial TCGA cancers. Predictions made by projection onto varimax PCA from FIG. 1A. For the two left boxes SCN scores are z-normalized w.r.t CRPC (leftmost box) and LUAD (middle box). For samples in the right box, SCN score is the value of PCv1 from projection onto FIG. 1A, z-score normalized by cancer type. For all boxes, samples greater than 3 standard deviations from the mean are highlighted as enlarged data points. Known cases of NETs in PAAD (6 of 8; 2 missed are just slightly subthreshold) and SCNCs in BLCA (3 of 4 known cases, missed known case is next sample subthreshold) are predicted correctly (red inset boxes). Cancer types in rightmost box are left-right sorted based on the average of top 3 scores per cancer type. B) Kaplan-Meier of overall survival for predicted SCN versus non-SCN cases from epithelial TCGA cancers (samples in panel A right box plus LUAD; PAAD, SCLC, NEPC and CRPC-Adeno not included) (see Abbreviations section). P value is calculated controlling for tumor type. C) Left: P values from a Cox regression survival analysis in individual cancers using the continuous SCN score. Right: Pan-cancer Cox regression using the continuous score, accounting for cancer type. D-G) TCGA BRCA hematoxylin and eosin (H&E) stained diagnostic slides of invasive ductal carcinoma (D; TCGA-D8-A1XD), small cell neuroendocrine carcinoma (E; TCGA-BH-A0HL), mixed tumor with components of invasive ductal carcinoma (lower left, green arrow) and small cell neuroendocrine carcinoma (upper right, blue arrow) (F; TCGA-E9-A245), mixed tumor with components of large cell neuroendocrine carcinoma (upper left, green arrow) and small cell neuroendocrine carcinoma (lower right, blue arrow) (G; TCGA-A1-A0SK). H) Rug plot and Kolmogorov-Smirnov enrichment p value of breast cases scored by pathologist for SCN features ordered by their proliferation-removed SCN score. I) Scatter plot of all samples in TCGA BRCA cohort. (x-axis: Proliferation removed SCN score; y-axis: Z-scored Chromogranin A expression. Cases above the dashed red line, x=3, were computationally predicted as SCN-like) (See also FIGS. 11, 12).

FIG. 4A-C. Metastases across multiple tissue types have increased expression of SCN features. A) Projection of lung, prostate, bladder normal tissue, primary and metastatic non-SCN samples, and SCN samples onto the PCA framework of FIG. 1A. Samples were grouped into the categories of normal tissue, primary non-SCN, metastatic non-SCN, and SCN (both primary and metastatic), and the centroids and 80% confidence regions are displayed. Metastatic samples have profiles closer to SCNCs. Here, the pancreatic, cervix, stomach, and thymus blue samples are those annotated with a neuroendocrine (NE)-related term in Robinson et al., 2017. B) Heatmap of canonical SCN markers (top) and key SCN transcription factors (bottom) for prostate normal, primary adeno, metastatic adeno, and metastatic SCN. Samples ordered from left to right by the sum z-score of the genes displayed. C) Pan-cancer projections onto FIG. 1A (data from TCGA normal samples, TCGA primary tumors, MET500 metastatic tumors, SCLC tumors (George et al.), and CRPC and NEPC tumors (Beltran et al.). Plotted are the PCV1 values, representing SCN score. The SKIN.met cohort has two sources, the TCGA and met500 databases. Wilcoxon-Mann-Whitney p values are shown comparing primary (orange) and metastatic (red) cases. (*p<0.05, **p<0.01, ***p<0.001, ****p<0.0001). (See also Figure S6).

FIG. 5A-E. Blood cancers have SCN-like gene and protein expression profiles and drug sensitivities. A) Cell line gene expression SCN score (component 1 score of projection on PLSR of lung adeno and SCN lines). Z-score adjusted across all cell line samples together. Cancer types top-bottom sorted based on average SCN score per type. Red box: Non-SCN annotated epithelial lines with strong SCN score. B) Protein profile of blood cell lines, from projection onto PCA of lung adeno (LUAD) and lung SCN (SCLC) lines. C) Drug sensitivity profile of blood cell lines, from projection onto PCA of IC50 values for lung adeno (LUAD) and lung SCN (SCLC) lines. Ellipses represent 80% confidence regions. In panels B and C, waterfall plots show binned gene expression SCN score of projected cell lines, showing that cell lines with transcriptional profiles closer to SCLC are associated with high Protein and Drug SCN scores, confirming that protein or drug sensitivity SCN scores are concordant with gene expression SCN scores. D) Heatmap of drugs with differential sensitivity between lung adeno and SCN cell lines (t-test p value<0.0001). Hierarchical clustering is done on IC50 measurements for lung adeno, lung SCN, and blood lines. E) Enrichment of drug targets for drugs more effective in lung adeno or SCN lines. Kolmogorov Smirnov test p values. (See also FIG. 14-15).

FIG. 6A-G. Validation of shared vulnerabilities based on genome-scale functional RNAi screens. A) Varimax-rotated PLSR model trained on the genome-scale RNAi sensitivity values for lung adeno (LUAD) and lung SCN (SCLC) cell lines. Ellipses represent 80% confidence regions. B) Prediction of small cell RNAi sensitivity profile for blood, SRBCTs, and all other cell lines in the dataset. SCN sensitivity score based on PLSRv component 1 from panel A. SRBCTs include neuroblastoma, medulloblastoma, rhabdomyosarcoma, Ewing's sarcoma, and Merkel cell carcinoma. C-F) Comparison of gene set expression rank to gene set sensitivity rank for lung SCN versus adeno, and blood versus non-blood, for gene sets containing selected keywords. Gene set RRHO scatter plots are subcategorized and colored by immune (C), lipid (D), neuro (E), and cell cycle (F) gene sets, with all other gene sets colored gray. Arrows in top left corner of individual panels indicate direction of significance (q<0.01) by Kolmogorov-Smirnov test (diagonal arrows indicate significance in both expression and sensitivity directions; Benjamani-Hochberg correction). G) Select genes with differential lung SCN versus adeno, and blood versus non-blood RNAi sensitivity. The y-axis (RNAi sensitivity) is the published Demeter score. Student's t-test p values. (*p<0.1, **p<0.01, ***p<0.001, ****p<0.0001). (See also FIG. 16).

FIG. 7A-F. Tumors recapitulate SCN and blood cell line sensitivity signatures. A) Scatter plot of t-values from t-test of true IC50 values of lung SNC (SCLC) versus lung adeno (LUAD) in cell lines (x-axis) and predicted IC50 values in tumors (y-axis) highlight shared sensitivities signatures of tumor and cell line SCLC cases. B) Comparison of cross validation model R2 values (cell line-based) to signed log p values of cell line lung SCN versus lung adeno true drug sensitivities (IC50). R2 values are from true cell line drug sensitivities versus cross validation-based predicted cell line sensitivities. Dashed lines are p value 0.05 (log p value=1.3). Model cross-validation performance improves for drugs with significant true differential sensitivities, showing that the models are built on relevant gene expression features. C) Real and predicted sensitivities for NPK76 (PLK3 inhibitor) and ABT.263 (BCL2 inhibitor). Y-axis is the log IC50. D) Projection of RNA-seq, elastic net-based predicted sensitivities of epithelial tumors onto PCA framework of SCLC and LUAD cell lines. More negative “Relative Sensitivity Scores” correspond to having more SCN like drug sensitivity profile. Asterisks (*) denote individual tumor type significance by Kolmogorov Smirnov test, NS=not significant. SCLC and LUAD tumor predicted sensitivity compared via a Wilcoxon-Mann-Whitney test (p<2.2×10-16). Combined p value calculated with Stouffer's test. E) Real and predicted sensitivities for NPK76 (PLK3 inhibitor) and ABT.263 (BCL2 inhibitor) for blood tumors. Y-axis is the log IC50. F) Projection of elastic net sensitivities of epithelial tumors and blood tumors onto PCA framework of SCLC and LUAD cell lines (open bracket) highlights blood cancers are enriched for small cell sensitivity profiles; combined p value from Stouffer's test. More negative “Relative Sensitivity Scores” correspond to having more SCN like drug sensitivity profile.

FIG. 8A-G. Gene expression signatures of three tissue types supports convergent expression profiles of small cell neuroendocrine cancers. Related to FIG. 1. A) Hierarchical clustering on Pearson's correlation using all genes. B) Heterogeneity of canonical neuroendocrine markers in SCNC tumors, with a representative set of non-SCNC tumors for comparison. C) Varimax-rotated PCA with proliferation and proliferation-related genes removed. Ellipses represent 80% confidence regions. D) Left: GSEA on proliferation-removed PCV1 loadings. Dashed lines mark FDR q-value<0.05 in each direction. Right: Bar chart of individual categories Kolmogorov-Smirnov test −log 10(p values) for enrichment in SCN (for Neuro, Cell cycle and Splicing) or in Non-SCN (for Immune and Adhesion). E) Projection of patient tumor biopsies onto lung cell lines. Lung cell lines are from the same batch, with these results thus mitigating concerns of a batch effect between patient tumor datasets. Ellipses represent 80% confidence regions. F) Melanoma (SKCM) tumors projected onto lung cell line-defined PC components. The neuroendocrine dedifferentiation signature is distinct from other dedifferentiation trajectories, such as melanoma dedifferentiation (clusters 1-4 from Tsoi et al.). G) GSEA of a t-test defined SCNC versus adenocarcinoma signature. Gene sets are ranked by NES score. Kolmogorov-Smirnov p values<2×10-16 for all.

FIG. 9A-B. Small cell neuroendocrine cancer convergence is reflected by epigenetic changes. Related to FIG. 2. A) Lung tumor methylation projected to lung cell line methylation. Cell lines are from the same batch, mitigating concerns of batch effect in tumor data derived from two different datasets. Ellipses represent 80% confidence regions. B) Rank signature overlap (RRHO) of PLSR component 1 loadings of matched sites averaged to gene loci across lung, prostate, and bladder. Only the subset of sites that matched across 450K microarray and RRBS sequencing platforms were used. The average rank of these ranked gene lists created independently on the 3 separate tissues were used in the enrichment analysis in FIG. 2E. Numbers shown are maximum −log 10(p values) of the RRHO heatmap.

FIG. 10A-G. Small cell neuroendocrine cancers of lung and prostate origin share DNA copy number alteration patterns. Related to FIG. 2. A) PCA on lung CCLE cell line DNA copy number alteration (CNA) profiles. (Side tracks are density plots of points along PC2.) B) Cell line PC1 score reflects degree of aneuploidy (integrated CNA (iCNA) score). C) Projection of lung tumors to cell line-defined PC components of panel A. LUAD category randomly down-sampled for clarity. Cell lines are from the same batch, mitigating batch effect concerns in the lung tumor data which are from two separate datasets. D) Projection of prostate tumors to cell line-defined PC components of panel A. E) PLSR of lung and prostate tumor CNA profiles, regressed on SCN or non-SCN status. LUAD category randomly down-sampled to match numbers in other categories. F) Genome-wide view of CNA patterns. Each row is a tumor biopsy sample: SCLC-red, LUAD-light green, NEPC-blue, CRPC-brown; SC-green, non-SC (NSC)-orange. G) Copy number changes consistently observed in both lung and prostate SCNC signatures, when each cancer type is initially analyzed by PLSR independently. Y-axis represents the mean of concordant PLSR loadings.

FIG. 11A-L. Tumors with SCN phenotype in breast cancer. Related to FIG. 3. Tumor tissues from the TCGA breast cancer (BRCA) cohort scored by pathologist review of diagnostic slides for SCN features. A-D) TCGA-AC-A2QH. The case was originally diagnosed as invasive ductal carcinoma. Careful examination of the digital picture available at the TCGA website revealed focal areas of SCNC. A) Low power view of the case. Yellow arrow indicates the junction of invasive ductal carcinoma and SCNC. B) Medium power view of the junction of invasive ductal carcinoma and SCNC. Blue arrow points to the component of invasive ductal carcinoma, while yellow arrow points to the component of SCNC. C) High power view of invasive ductal carcinoma region. D) High power view of SCNC region. E-H) TCGA-A7-A13D. The case was originally also diagnosed as invasive ductal carcinoma. Careful examination of the digital picture available at the TCGA website revealed focal areas of SCNC. E) Low power view of the case. Yellow arrow indicates the junction of invasive ductal carcinoma and SCNC. F) Medium power view of the junction of invasive ductal carcinoma and SCNC. Blue arrow points to the component of invasive ductal carcinoma, while yellow arrow points to the component of SCNC. G) High power view of invasive ductal carcinoma region. H) High power view of SCNC region. I) Boxplots of SCN Score by PAM50 breast subtype. Dashed red line (y=3) is threshold for calling tumor SCN-like. J) Scatter plot of REST Score vs SCN Score for 4 tumor types (R is Pearson's correlation). K-L) Violin plot of Pearson's R from correlation of REST Score (K) or REST gene expression (L) to SCN score for TCGA cancers (dots are the individual tumor type R values). P value from one way t-test (null hypothesis mean=0), t is the signed t statistic. In most cancer types in the TCGA cohort, higher SCN scores are positively correlated with the expression of REST target genes, which supports the loss of REST as a transcriptional repressor in cancers that have an SCN component. The expression of the REST gene itself is also somewhat positively correlated with SCN score in the majority of cancers types (whereas the paradigm is that it should be negatively correlated owing to its function in downregulation of neural genes). This finding down weights REST's potential as a direct marker of non-NE tissues in samples with mixed histology. One possibility for this discrepancy is that REST function can be lost in multiple ways such as by mutation, (Mahamdallie et al., 2015), or by truncation/alternative splicing that abrogates its function (Chen and Miller, 2018)—both of which would not require concordant changes in transcription levels.

FIG. 12A-C. Genetic mutations associated with the SCN phenotype. Related to FIG. 3. A) Gene mutation association with continuous SCN score in lung and prostate tumors. Signed log p values are positive (negative) when associated with the small cell neuroendocrine state (non-SCN state). Red line indicates a q-value of 0.1. B) Pan-epithelial cancer gene mutation association with SCN score in primary tumors. Mutational status is the dependent variable, conditioned on neuroendocrine score and tissue type. Dashed red line indicates a q-value of 0.1. C) Gene mutation association by individual cancer type across all TCGA cancers with data available. Dashed red line indicates a nominal p value of 0.05. Cancers not shown have no genes that met this threshold (COADREAD, UCEC, PAAD).

FIG. 13A-D. Metastatic carcinomas display a convergent SCN trajectory with a neuronal signature. Related to FIG. 4. A) Projection of adenocarcinoma and SCN metastases onto PCA framework of FIG. 1A (black dots are FIG. 1A samples). Pan-cancer metastatic SCNs project onto the SCN space defined by lung and prostate. B) Expression levels of top 50 SCN signature gene loadings (prostate and lung FIG. 1A-defined) in prostate tissue and cancer samples. Samples are sorted by the sum z-score across these 50 genes. C) Separation of proliferation signal and SCN signal. Note some SCN samples (blue) are either primary (circle) or metastatic (square) biopsies. This approach highlighted a unique distinction in the pancreatic neuroendocrine tumors, in which both primary and metastatic tumors had equal expression of the neuronal program, but could be distinguished by their expression of the proliferation signature. Here, the pancreatic, cervix, stomach, and thymus blue samples are those annotated with a neuroendocrine (NE)-related term in Robinson et al., 2017. Wilcoxon-Mann-Whitney test p values are shown comparing primary (orange) and metastasis (red). (*p<0.05, **p<0.01, ***p<0.001, ****p<0.0001). D) Additional low grade neuroendocrine tumors from the pancreas, rectum, and small intestine. A different number of genes was used due to differences in data source and processing. Thus, lung and prostate SCN, and TCGA PAAD and TCGA pancreatic NETs shown in S6C are replotted for frame of reference.

FIG. 14A-B. Blood cancers share expression profiles with SCNCs. Related to FIG. 5. A) SCN score of blood and SRBCT patient biopsies by projection onto FIG. 1A framework (non-z-score normalized to keep on common scale). B) Heatmap of significantly differentially expressed proteins between lung adeno (LUAD) and lung SCN (SCLC) cell lines (t-test p value<0.01). Hierarchical clustering is done on measurements for lung adeno, lung SCN, and blood lines. Values were imputed for proteins lacking measurements as described in methods. Cell lines with >25% imputed values for the proteins shown were removed.

FIG. 15A-F. Shared drug sensitivities in SCNC, SRBCTs, and SCN-like epithelial cancer cell lines. Related to FIG. 5. A) PCA of protein profiles for lung adeno (LUAD) and lung SCN (SCLC) lines, and projection of all other cell lines, with SRBCTs highlighted as a group. SRBCTs include neuroblastoma, medulloblastoma, rhabdomyosarcoma, and Ewing's sarcoma. Ellipses represent 80% confidence regions. B) PCA of drug sensitivity IC50 for lung adeno and SCN lines, and projection of all other cell lines, with SRBCTs highlighted as a group. C) Individual drug sensitivities for selected drugs with high differential sensitivity (p value<0.0001) in the lung SCN versus adeno comparison, and blood versus non-blood comparison. Individual blood cancer types are shown, which were all grouped into the BLOOD category in panels A and B. Samples marked BLOOD in this panel are blood cancer lines of unclear subtype. Drug targets shown in parentheses of axes labels. Y-axis is the log IC50. D) Left: PC2 (from FIG. 5C) of drug sensitivity data on lung cell lines with culture-based growth characteristic indicated. Top right: Scatterplot oft-test signed log p values of SCLC (all lines) vs. LUAD (x-axis) and blood vs. other (y-axis) drug sensitivity profile comparisons (Pearson's R=0.68). Bottom right: As in left panel but restricting SCLC samples to only those that grow adherent (Pearson's R=0.69). The correlation coefficient of the two x-axes, “SCLC (suspension only) vs. LUAD” and “SCLC (adherent only) vs. LUAD”, is R=0.81. Note: almost all LUAD lines grow adherent (94%). (n=20 adherent SCLC, 6 semi-adherent SCLC, 38 suspension SCLC; 168 blood samples, 638 other (non-blood, non-lung, non-SRBCT), 58 adherent LUAD). Analysis of the shRNA knockdown sensitivity data likewise shows correlation between SCLC and blood knockdown sensitivity, even when only adherent SCLCs are used. The correlation of the blood vs other knockdown sensitivity signature to the SCLC vs LUAD signature is R=0.32 using only adherent SCLCs, R=0.4 using only suspension SCLCs, and R=0.43 using all SCLCs (n=11 adherent, n=2 semi-adherent, n=10 suspension SCLCs). E) Correlation (Pearson) between cell line drug sensitivity SCN score and RNA expression SCN score for epithelial cancer cell lines. Cell lines with larger RNA and drug scores are more SCN-like. (Combined p value for all epithelial cancers except LUAD and SCLC using weighted-Stouffer's test). The first panel shows the correlation coefficient and p values for both LUAD and SCLC. The individual values are r=−0.14, p=0.32, and r=−0.51, p=0.0012, respectively. F) Mean cell line drug sensitivity SCN score versus mean expression SCN score for epithelial tissue cell lines. Inset, inclusion of the mean from lung small cell lines (red circle region corresponds to the main graph). (*p<0.05, **p<0.01, ***p<0.001, ****p<0.0001).

FIG. 16A-E. Validation of shared vulnerabilities based on genome-scale functional RNAi screens. Related to FIG. 6. A) Varimax-rotated PLSR (PLSRV) of blood versus non-blood samples (all other cell lines except SCLC and SRBCT). Ellipses represent 80% confidence regions. B) Projection of LUAD, SCLC and SRBCT cell lines onto PLSRV component 1 of panel A. C) Enrichment or de-enrichment of categories of gene sets, based on co-ranked blood and SCLC RNAi sensitivity gene set signatures. Dotted line in the p value bar plot indicates nominal Kolmogorov-Smirnov p value=0.01. D-E) RRHO scatter plots of blood and lung SCN sensitivities by genes (D) and by gene set (E). Gene set RRHO scatter plots are subcategorized and colored by immune, lipid, neuro, and cell cycle gene sets, with all other gene sets colored gray (left: sensitivity based, right: expression based).

FIG. 17A-C. Prediction of samples with SCN features using a logistic regression model with LASSO (LRL), using top/bottom 100. Using the top 100 and bottom 100 genes from the loadings of FIG. 1A as input to the LRL, a search grid of 100 values of the tuning parameter were generated (final model contained 47 genes). Predictions were performed on A) the original training set (100% accuracy) or B) lung cell lines in the CCLE (95.2% accuracy). (Dashed lines represent the value of the tuning parameter used for prediction, x-axis=logarithm of lambda tuning parameter (reflecting the number of genes used), y-axis=accuracy.) C) Genes in the final model under these parameters. Different sets of final genes can be similarly informative.

FIG. 18A-C. Prediction of samples with SCN features using a logistic regression model with LASSO (LRL), using top/bottom 500. Using the top 500 and bottom 500 genes from the loadings of FIG. 1A as input to the LRL, a search grid of 100 values of the tuning parameter were generated (final model contained 41 genes). Predictions were made on the A) the original training set (100% accuracy) or B) lung cell lines in the CCLE (95.2% accuracy). (Dashed lines represent the value of the tuning parameter used for prediction, x-axis=logarithm of lambda tuning parameter (reflecting the number of genes used), y-axis=accuracy.) C) Genes in the final model under these parameters. Different sets of final genes can be similarly informative.

### DETAILED DESCRIPTION OF THE INVENTION

In this disclosure, by defining the molecular signatures shared between SCN cancers from different tissue types, the inventors have identified molecular networks, key transcription factors, and gene mutations that drive a convergent SCN phenotype. The implications of identifying this molecular spectrum of SCN cancers is apparent in functional data on drug response and gene dependency, which further supports the value of identifying SCN-like cases in clinical assessment. The molecular profiling-based SCN scoring identified cases with SCN or neuroendocrine features across numerous purportedly non-SCN primary tumors. These cases may not have been reported in the original pathology reports due to the fact that their SCN component is typically focal, and the conventional thinking that SCN is rare in breast and other epithelial tumors. Epithelial tumor types, such as breast cancer, in which SCN tumors rarely occur but have poor prognosis, present statistical challenges in advancing care through clinical trials, and thus cross-tissue learning supported by shared molecular profiles will likely be required. In sum, the current disclosure relates to methods for identifying and classifying patients with SCN and treatments that may be particularly effective to treat those patients.

### I. Definitions

The term “substantially the same”, “not significantly different”, or “within the range” refers to a level of expression that is not significantly different than what it is compared to. Alternatively, or in conjunction, the term substantially the same refers to a level of expression that is less than 2, 1.5, or 1.25 fold different than the expression level it is compared to or less than 20, 15, 10, or 5% difference in expression.

By “subject” or “patient” is meant any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

The term “primer” or “probe” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred. A probe may also refer to a nucleic acid that is capable of hybridizing by base complementarity to a nucleic acid of a gene of interest or a fragment thereof.

As used herein, “increased” or “elevated” or “decreased” refers to expression level of a biomarker in the subject's sample as compared to a reference level representing the same biomarker or a different biomarker. In certain aspects, the reference level may be a reference level of expression from a non-cancerous tissue from the same subject or from a cancerous tissue that is not a small cell neuroendocrine cancer. Alternatively, the reference level may be a reference level of expression from a different subject or group of subjects. For example, the reference level of expression may be an expression level obtained from a sample (e.g., a tissue, fluid or cell sample) of a subject or group of subjects without cancer, with cancer, with SCN or SRBCT cancer, with a can that is not SCN or SRBCT, with a cancer that has not undergone transdifferentiation, or an expression level obtained from a non-cancerous tissue of a subject or group of subjects with cancer. The reference level may be a single value or may be a range of values. The reference level of expression can be determined using any method known to those of ordinary skill in the art. The reference level may also be depicted graphically as an area on a graph. In certain embodiments, a reference level is a normalized level.

The term “determining” or “evaluating” as used herein may refer to directly or indirectly measuring, quantitating, or quantifying (either qualitatively or quantitatively).

### II. Cancer Therapies

Embodiments of the disclosure relate to administration of cancer therapies. In addition to the cancer therapies listed in Table 4A and 4B, it is contemplated the cancer therapies described below, such as an immunotherapy, virus, polysaccharide, neoantigen, chemotherapy, radiotherapy, surgery, or other agent described below may be used alone or in combination to treat SCN and/or SRBCT tumors.

A. Immunotherapy

In some embodiments, the cancer therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Embodiments of the disclosure may include administration of immune checkpoint inhibitors (also referred to as checkpoint inhibitor therapy), which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

In some embodiments, the immunotherapy comprises dendritic cell therapy. Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. CAR-T Cell Therapy

In some embodiments, the cancer therapy comprises CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

In some embodiments, the immunotherapy comprises cytokine therapy. Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

In some embodiments, the immunotherapy comprises adoptive T cell therapy. Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death. [60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

C. Polysaccharides

In some embodiments, the cancer therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the cancer therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the cancer therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the cancer therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

In some embodiments, the cancer therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### III. ROC Analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making. ROC analysis provides a tool for creating cut-off values to partition patient populations into high expression and low expression of certain biomarkers.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety.

### IV. Sample Preparation

In certain aspects, methods involve obtaining a biological sample from a subject and/or evaluating a biological sample. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. Evaluating Levels of Biomarkers

In certain aspects a meta-analysis of expression or activity can be performed. In statistics, a meta-analysis combines the results of several studies that address a set of related research hypotheses. This is normally done by identification of a common measure of effect size, which is modeled using a form of meta-regression. Generally, three types of models can be distinguished in the literature on meta-analysis: simple regression, fixed effects meta-regression and random effects meta-regression. Resulting overall averages when controlling for study characteristics can be considered meta-effect sizes, which are more powerful estimates of the true effect size than those derived in a single study under a given single set of assumptions and conditions. A meta-gene expression value, in this context, is to be understood as being the median of the normalized expression of a biomarker gene or activity. Normalization of the expression of a biomarker gene is preferably achieved by dividing the expression level of the individual marker gene to be normalized by the respective individual median expression of this marker genes, wherein said median expression is preferably calculated from multiple measurements of the respective gene in a sufficiently large cohort of test individuals. The test cohort preferably comprises at least 3, 10, 100, 200, 1000 individuals or more including all values and ranges thereof. Dataset-specific bias can be removed or minimized allowing multiple datasets to be combined for meta-analyses (See Sims et al. BMC Medical Genomics (1:42), 1-14, 2008, which is incorporated herein by reference in its entirety).

The calculation of a meta-gene expression value is performed by: (i) determining the gene expression value of at least two, preferably more genes (ii) “normalizing” the gene expression value of each individual gene by dividing the expression value with a coefficient which is approximately the median expression value of the respective gene in a representative breast cancer cohort (iii) calculating the median of the group of normalized gene expression values.

A gene shall be understood to be specifically expressed in a certain cell type if the expression level of the gene in the cell type is at least about 2-fold, 5-fold, 10-fold, 100-fold, 1000-fold, or 10000-fold higher (or any range derivable therein) than in a reference cell type, or in a mixture of reference cell types. Reference cell types include non-cancerous tissue cells or a heterogenous population of cancers.

In certain algorithms a suitable threshold level is first determined for a marker gene. The suitable threshold level can be determined from measurements of the marker gene expression in multiple individuals from a test cohort. The median expression of the marker gene in said multiple expression measurements is taken as the suitable threshold value.

Comparison of multiple marker genes with a threshold level can be performed as follows: 1. The individual marker genes are compared to their respective threshold levels. 2. The number of marker genes, the expression level of which is above their respective threshold level, is determined. 3. If a marker genes is expressed above its respective threshold level, then the expression level of the marker gene is taken to be “above the threshold level”.

Some embodiments include determining that a measured expression level is higher than, lower than, increased relative to, decreased relative to, equal to, or within a predetermined amount of a reference expression level. In some embodiments, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain gene or miRNA at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. This can be the case, for example, when a classifier is used to identify the molecular subtype of a metastasis. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene or miRNA expression levels to a mean expression levels or a reference expression levels, the comparison is to be made on a gene-by-gene and miRNA-by-miRNA basis. For example, if the expression levels of gene A, gene B, and miRNA X in a patient's cancerous sample are measured, a comparison to mean expression levels in cancerous samples of a cohort of patients would involve: comparing the expression level of gene A in the patient's cancerous sample with the mean expression level of gene A in cancerous samples of the cohort of patients, comparing the expression level of gene B in the patient's sample with the mean expression level of gene B in samples of the cohort of patients, and comparing the expression level of miRNA X in the patient's metastasis with the mean expression level of miRNA X in cancerous samples of the cohort of patients. Comparisons that involve determining whether the expression level measured in a patient's sample is within a predetermined amount of a mean expression level or reference expression level are similarly done on a gene-by-gene and miRNA-by-miRNA basis, as applicable.

### VI. Nucleic Acid Assays

Aspects of the methods include assaying nucleic acids to determine expression levels. Arrays can be used to detect differences between two samples. Specifically contemplated applications include identifying and/or quantifying differences between bacterial populations from a sample that is normal and from a sample that is not normal, between a cancerous condition and a non-cancerous condition, or between two differently treated samples. Also, microbiome profiles may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids, their activities, and their effects. Such assays include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### VII. Therapeutic Methods

The current methods and compositions relate to methods for treating cancer. In some embodiments, the cancer comprises a solid tumor. In some embodiments, the cancer is non-lymphatic. In some embodiments, the cancer is an epithelial cancer. In some embodiments, the caner excludes a hematological cancer.

The compositions of the disclosure may be used for in vivo, in vitro, or ex vivo administration. The route of administration of the composition may be, for example, intratumoral, intracutaneous, subcutaneous, intravenous, intralymphatic, and intraperitoneal administrations. In some embodiments, the administration is intratumoral or intralymphatic or peri-tumoral. In some embodiments, the compositions are administered directly into a cancer tissue or a lymph node.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VIII. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed, for example, a first cancer treatment is “A” and a second cancer treatment is

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX. Kits

Certain aspects of the present invention also concern kits containing compositions of the invention or compositions to implement methods of the invention. In some embodiments, kits can be used to evaluate one or more biomarkers, such as one or more SCN or SRBCT biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### X. Tables

TABLE 1
SCN gene signature weights

| | SCN |
|---|---|
| | signature |
| Gene | weight |
| TARDBP | 0.050046 |
| PIK3R3 | 0.049087 |
| DPYSL5 | 0.044226 |
| TMBIM4 | 0.042018 |
| PIK3R2 | 0.041165 |
| INSM1 | 0.040721 |
| TIMM10B | 0.039219 |
| KIF1A | 0.038317 |
| PSMA1 | 0.037698 |
| STXBP5L | 0.037609 |
| CHGA | 0.036387 |
| PAPD7 | 0.036124 |
| ELAVL3 | 0.035688 |
| DLL3 | 0.034705 |
| ASCL1 | 0.034372 |
| PI4K2B | 0.034356 |
| ZIC5 | 0.034346 |
| DLK1 | 0.034262 |
| SEZ6 | 0.034028 |
| FOXG1 | 0.033796 |
| GNG4 | 0.033750 |
| ATCAY | 0.033655 |
| ZIC2 | 0.033534 |
| RAB3B | 0.033273 |
| RIMS2 | 0.033236 |
| ACTL6B | 0.033103 |
| ZNF286A | 0.033099 |
| ONECUT2 | 0.032991 |
| INA | 0.032957 |
| NRSN1 | 0.032855 |
| PAGR1 | 0.032600 |
| CPLX2 | 0.032351 |
| BEST3 | 0.032243 |
| CELF3 | 0.032012 |
| DCX | 0.031961 |
| ST18 | 0.031529 |
| CACNA1A | 0.031512 |
| XKR7 | 0.031507 |
| LHX2 | 0.031425 |
| CACNA1B | 0.031268 |
| SYT4 | 0.031045 |
| C17orf100 | 0.031021 |
| PCSK1 | 0.030989 |
| TAGLN3 | 0.030809 |
| SOX11 | 0.030603 |
| FAM57B | 0.030310 |
| CHRNB2 | 0.030170 |
| NKX2-2 | 0.030022 |
| KCNC1 | 0.029939 |
| ISL1 | 0.029906 |
| HEPACAM2 | 0.029840 |
| ST8SIA3 | 0.029799 |
| DLX6 | 0.029756 |
| SCG3 | 0.029402 |
| PHF21B | 0.029396 |
| DLX1 | 0.029377 |
| ACSL6 | 0.029205 |
| SPATA13 | 0.029188 |
| NOL4 | 0.029159 |
| SEZ6L | 0.029159 |
| CDK5R2 | 0.029034 |
| CAMK2B | 0.028906 |
| B4GALNT4 | 0.028815 |
| TMEM179 | 0.028799 |
| CHGB | 0.028725 |
| RGS5 | 0.028715 |
| KCNH5 | 0.028658 |
| KIF5C | 0.028640 |
| MAST1 | 0.028625 |
| TMEM151B | 0.028579 |
| ZBTB8B | 0.028501 |
| MYT1 | 0.028396 |
| VGF | 0.028366 |
| RALYL | 0.028332 |
| SLC7A14 | 0.028238 |
| SYT13 | 0.028114 |
| UNC13A | 0.027888 |
| ST8SIA5 | 0.027832 |
| CAMK2N2 | 0.027811 |
| SCGN | 0.027799 |
| MPPED1 | 0.027698 |
| UNC80 | 0.027571 |
| NRXN1 | 0.027371 |
| HRH3 | 0.027336 |
| ELAVL4 | 0.027325 |
| CNTNAP2 | 0.027288 |
| SYT14 | 0.027228 |
| OGDHL | 0.027225 |
| ZMAT4 | 0.027169 |
| TCERG1L | 0.026964 |
| ZDHHC22 | 0.026950 |
| GRM4 | 0.026729 |
| AMH | 0.026692 |
| SRRM4 | 0.026690 |
| DGKB | 0.026580 |
| FOXN4 | 0.026556 |
| NEUROD1 | 0.026519 |
| PCDH8 | 0.026511 |
| RIMBP2 | 0.026498 |
| SULT4A1 | 0.026466 |
| AMER3 | 0.026430 |
| TMEFF1 | 0.026347 |
| NKAIN1 | 0.026332 |
| BTBD17 | 0.026300 |
| SCN3A | 0.026154 |
| SVOP | 0.026100 |
| POU4F1 | 0.026037 |
| HOXD13 | 0.025985 |
| CBLN2 | 0.025911 |
| TRPM8 | 0.025897 |
| RUNDC3A | 0.025835 |
| IDS | 0.025572 |
| GRIK3 | 0.025522 |
| FSD1 | 0.025345 |
| HOXB9 | 0.025242 |
| HOXD11 | 0.025228 |
| BEX1 | 0.025117 |
| SLC6A17 | 0.025088 |
| CLN3 | 0.024931 |
| CRYBA2 | 0.024927 |
| NGB | 0.024816 |
| KCNH7 | 0.024805 |
| KIF5A | 0.024804 |
| 43535 | 0.024730 |
| COL2A1 | 0.024673 |
| TUBB2B | 0.024657 |
| UNC79 | 0.024609 |
| BARX1 | 0.024534 |
| TRIM9 | 0.024446 |
| POU3F2 | 0.024437 |
| KCNB2 | 0.024396 |
| KCNK9 | 0.024392 |
| DRD2 | 0.024382 |
| SYT5 | 0.024358 |
| KCNA1 | 0.024348 |
| C12orf56 | 0.024274 |
| PRAME | 0.024245 |
| SOX1 | 0.024178 |
| PEX5L | 0.024159 |
| CAMKV | 0.024143 |
| SALL1 | 0.024140 |
| CABP7 | 0.024106 |
| BRSK2 | 0.024054 |
| PHYHIPL | 0.024008 |
| DLX2 | 0.023887 |
| PTPRN | 0.023751 |
| GAD1 | 0.023747 |
| C11orf71 | 0.023739 |
| FUT9 | 0.023668 |
| KCNH8 | 0.023608 |
| ELOVL2 | 0.023538 |
| GPC2 | 0.023503 |
| EPHA8 | 0.023369 |
| COL22A1 | 0.023360 |
| NXPH4 | 0.023348 |
| KCNH6 | 0.023265 |
| NKAIN2 | 0.023157 |
| GPR6 | 0.023028 |
| HES6 | 0.023018 |
| DLX5 | 0.022900 |
| AP3B2 | 0.022896 |
| CELSR3 | 0.022882 |
| TERT | 0.022839 |
| SNAP91 | 0.022802 |
| SALL3 | 0.022753 |
| VWA5B2 | 0.022720 |
| ECSCR | 0.022664 |
| E2F7 | 0.022589 |
| RTBDN | 0.022574 |
| ZACN | 0.022539 |
| GAD2 | 0.022517 |
| TPH1 | 0.022478 |
| CACNA1E | 0.022469 |
| CALML3 | 0.022441 |
| CNTNAP5 | 0.022435 |
| NEUROD4 | 0.022395 |
| 43528 | 0.022364 |
| CADPS | 0.022363 |
| GRIA2 | 0.022350 |
| HOXD10 | 0.022343 |
| TMEM145 | 0.022342 |
| SCN8A | 0.022120 |
| KIF18B | 0.022106 |
| ASPM | 0.022076 |
| PPM1E | 0.022048 |
| MRPS17 | 0.022007 |
| C1QL1 | 0.021997 |
| HMX2 | 0.021955 |
| KCNJ6 | 0.021950 |
| FBXO43 | 0.021870 |
| RIPPLY3 | 0.021866 |
| DEPDC1 | 0.021850 |
| SLC6A5 | 0.021804 |
| MYT1L | 0.021779 |
| NELL1 | 0.021770 |
| CTNNA2 | 0.021751 |
| CCNE2 | 0.021716 |
| SHOX2 | 0.021672 |
| GRP | 0.021669 |
| RNF183 | 0.021577 |
| TROAP | 0.021568 |
| CHD5 | 0.021561 |
| INSRR | 0.021543 |
| SLC35D3 | 0.021542 |
| GDAP1L1 | 0.021492 |
| SCGB1D2 | 0.021439 |
| TMEM178B | 0.021305 |
| CDH7 | 0.021291 |
| SIX3 | 0.021267 |
| CASKIN1 | 0.021263 |
| TRIM67 | 0.021229 |
| B3GALT5 | 0.021163 |
| SLCO5A1 | 0.021156 |
| HGFAC | 0.021152 |
| FBF1 | 0.021142 |
| EPHA7 | 0.021141 |
| ADCYAP1 | 0.021105 |
| NUF2 | 0.021039 |
| DIRAS2 | 0.021015 |
| SCN2A | 0.021014 |
| SNAP25 | 0.021013 |
| DPF1 | 0.020945 |
| UBE2QL1 | 0.020938 |
| RAD54L | 0.020914 |
| GP1BB | 0.020880 |
| CRABP1 | 0.020879 |
| CPLX3 | 0.020824 |
| NAT16 | 0.020801 |
| BRSK1 | 0.020795 |
| KIF14 | 0.020782 |
| HJURP | 0.020754 |
| KCNK12 | 0.020719 |
| ADAMTS18 | 0.020714 |
| MNX1 | 0.020709 |
| UNC5A | 0.020679 |
| COCH | 0.020621 |
| TOP2A | 0.020601 |
| ZNF695 | 0.020585 |
| FAM64A | 0.020571 |
| AMER2 | 0.020562 |
| NPPA | 0.020538 |
| CENPF | 0.020517 |
| EEF1A2 | 0.020507 |
| ELAVL2 | 0.020501 |
| DEPDC1B | 0.020444 |
| RIPPLY2 | 0.020407 |
| STMN2 | 0.020406 |
| TEX15 | 0.020386 |
| APC2 | 0.020370 |
| MSI1 | 0.020336 |
| NEURL1 | 0.020268 |
| DMRTA2 | 0.020162 |
| SHISA9 | 0.020137 |
| ABCC8 | 0.020127 |
| SCRT1 | 0.020095 |
| CCDC150 | 0.020087 |
| FAM83F | 0.020080 |
| KCNK10 | 0.020062 |
| HOXB8 | 0.020056 |
| ATOH7 | 0.020053 |
| TTK | 0.020048 |
| NEUROG2 | 0.020024 |
| TTBK1 | 0.019983 |
| STMN4 | 0.019952 |
| PDIA2 | 0.019920 |
| GLYATL1 | 0.019856 |
| POTEI | 0.019788 |
| DLGAP5 | 0.019781 |
| PBK | 0.019738 |
| DLGAP3 | 0.019721 |
| NXPH1 | 0.019719 |
| TMEM63C | 0.019718 |
| LRTM2 | 0.019709 |
| FAM111B | 0.019709 |
| HOXA10 | 0.019707 |
| FOXD1 | 0.019695 |
| CENPA | 0.019689 |
| DPYSL4 | 0.019676 |
| CNTN2 | 0.019641 |
| SHD | 0.019618 |
| HOXA13 | 0.019618 |
| FAM163B | 0.019603 |
| MEX3A | 0.019558 |
| OR51E2 | 0.019553 |
| CALCB | 0.019531 |
| SP8 | 0.019520 |
| WNK2 | 0.019514 |
| RAB26 | 0.019503 |
| PPFIA4 | 0.019480 |
| SPOCK1 | 0.019470 |
| RBFOX1 | 0.019457 |
| MATR3 | 0.019445 |
| MAGEA3 | 0.019440 |
| SAPCD2 | 0.019393 |
| ESPL1 | 0.019386 |
| HOXD4 | 0.019380 |
| SLC18A1 | 0.019365 |
| ATP1A3 | 0.019364 |
| GRM8 | 0.019360 |
| FOXD3 | 0.019355 |
| INHBE | 0.019344 |
| NDST4 | 0.019336 |
| BSN | 0.019310 |
| CER1 | 0.019298 |
| HIST1H2BH | 0.019294 |
| KIF4A | 0.019288 |
| GRM2 | 0.019262 |
| FRRS1L | 0.019244 |
| SOWAHA | 0.019237 |
| SCG2 | 0.019209 |
| ACRV1 | 0.019190 |
| KCNQ2 | 0.019163 |
| UHRF1 | 0.019149 |
| LCN15 | 0.019148 |
| RDM1 | 0.019132 |
| BUB1B | 0.019102 |
| CALB1 | 0.019033 |
| RGS17 | 0.019032 |
| CDC25C | 0.019021 |
| CLGN | 0.018981 |
| IQGAP3 | 0.018974 |
| LHFPL5 | 0.018970 |
| CLVS2 | 0.018965 |
| CNIH2 | 0.018937 |
| ATP2B3 | 0.018936 |
| CCDC177 | 0.018929 |
| UBE2C | 0.018927 |
| NCAM1 | 0.018922 |
| POLQ | 0.018917 |
| NEK2 | 0.018907 |
| TMSB15A | 0.018905 |
| NDC80 | 0.018901 |
| PCP4 | 0.018866 |
| MKRN3 | 0.018863 |
| MELK | 0.018847 |
| GTSE1 | 0.018820 |
| SKA1 | 0.018761 |
| EZH2 | 0.018753 |
| SLIT1 | 0.018740 |
| ST8SIA2 | 0.018740 |
| GABRQ | 0.018736 |
| CBS | 0.018664 |
| UCHL1 | 0.018653 |
| NMU | 0.018651 |
| LRRC7 | 0.018635 |
| MYBL2 | 0.018623 |
| DDC | 0.018576 |
| EXO1 | 0.018534 |
| FGF17 | 0.018531 |
| ARHGAP33 | 0.018529 |
| SIX2 | 0.018518 |
| SRSF12 | 0.018518 |
| TPX2 | 0.018506 |
| CDH2 | 0.018498 |
| NTN3 | 0.018492 |
| EMX1 | 0.018483 |
| NEIL3 | 0.018461 |
| OR51E1 | 0.018440 |
| NEUROG3 | 0.018429 |
| KCNK16 | 0.018396 |
| CKAP2L | 0.018388 |
| E2F2 | 0.018386 |
| KIF2C | 0.018371 |
| KIF15 | 0.018341 |
| ELOOL4 | 0.018340 |
| 43711 | 0.018318 |
| HIST1H2BG | 0.018297 |
| PROX1 | 0.018274 |
| L1CAM | 0.018270 |
| ACBD7 | 0.018253 |
| OPRD1 | 0.018246 |
| SRPK3 | 0.018213 |
| PAH | 0.018204 |
| RPRM | 0.018194 |
| SLC45A2 | 0.018157 |
| PIF1 | 0.018150 |
| GPRIN1 | 0.018138 |
| NCAPG | 0.018130 |
| CRHR1 | 0.018103 |
| SHISA8 | 0.018100 |
| MED12L | 0.018065 |
| CCKBR | 0.018033 |
| MTHFS | 0.018020 |
| DDX25 | 0.017979 |
| GHRH | 0.017948 |
| LRFN2 | 0.017940 |
| C19orf45 | 0.017932 |
| NCAN | 0.017921 |
| DSCAM | 0.017910 |
| MAGEA6 | 0.017900 |
| HPCA | 0.017899 |
| ASXL3 | 0.017861 |
| SPAG5 | 0.017821 |
| NUSAP1 | 0.017814 |
| C16orf59 | 0.017808 |
| RHBDL3 | 0.017805 |
| CDCA3 | 0.017797 |
| FAM155B | 0.017792 |
| BIRC5 | 0.017769 |
| RCOR2 | 0.017761 |
| CALCA | 0.017757 |
| SKA3 | 0.017743 |
| FLRT1 | 0.017734 |
| KLK2 | 0.017733 |
| ADD2 | 0.017732 |
| XRCC2 | 0.017706 |
| ELFN2 | 0.017702 |
| SYNPR | 0.017695 |
| KLK12 | 0.017690 |
| CLSPN | 0.017688 |
| SIX6 | 0.017653 |
| KCNT1 | 0.017649 |
| OTP | 0.017637 |
| ANK1 | 0.017597 |
| BRINP2 | 0.017595 |
| KDM5D | 0.017573 |
| MAGEA2 | 0.017564 |
| CEL | 0.017536 |
| KCNC2 | 0.017532 |
| TICRR | 0.017525 |
| ZNF711 | 0.017519 |
| HOXD3 | 0.017504 |
| SPC24 | 0.017477 |
| GRIK5 | 0.017450 |
| PCSK1N | 0.017441 |
| SLC17A6 | 0.017424 |
| RGS7 | 0.017418 |
| ALX3 | 0.017411 |
| CDC20 | 0.017377 |
| NEB | 0.017365 |
| WNK3 | 0.017353 |
| ADCY2 | 0.017343 |
| MKI67 | 0.017323 |
| ORC6 | 0.017320 |
| SPC25 | 0.017314 |
| FAM72C | 0.017257 |
| CELF4 | 0.017256 |
| AURKB | 0.017243 |
| DNASE1L2 | 0.017238 |
| CDCA2 | 0.017232 |
| CYB561D2 | 0.017220 |
| INSM2 | 0.017207 |
| SLC38A11 | 0.017198 |
| RECQL4 | 0.017194 |
| SPTBN4 | 0.017177 |
| KIFC1 | 0.017161 |
| RAX | 0.017156 |
| GAP43 | 0.017144 |
| CDK5R1 | 0.017131 |
| SYT1 | 0.017092 |
| DPY19L2 | 0.017090 |
| T | 0.017089 |
| SCML2 | 0.017083 |
| MCM10 | 0.017074 |
| KIAA1549L | 0.017069 |
| KREMEN2 | 0.017066 |
| OTOG | 0.017059 |
| CCNB2 | 0.017029 |
| PPP2R2C | 0.017019 |
| ZYG11A | 0.017019 |
| CENPE | 0.017014 |
| USP41 | 0.016986 |
| SRRM3 | 0.016932 |
| HCN3 | 0.016911 |
| SP9 | 0.016909 |
| PRC1 | 0.016879 |
| TFAP2B | 0.016877 |
| FSTL5 | 0.016874 |
| CDC45 | 0.016862 |
| CDKN3 | 0.016858 |
| ERCC6L | 0.016858 |
| MGAT5B | 0.016838 |
| HIST1H2BO | 0.016831 |
| PGAM2 | 0.016820 |
| HELLS | 0.016803 |
| GFY | 0.016782 |
| HOXA9 | 0.016780 |
| HIST1H4E | 0.016766 |
| CDCA8 | 0.016753 |
| ADGRD2 | 0.016753 |
| SHISA7 | 0.016741 |
| RTL1 | 0.016737 |
| DRAXIN | 0.016727 |
| BRIP1 | 0.016714 |
| GINS2 | 0.016711 |
| GPR12 | 0.016698 |
| WDR62 | 0.016689 |
| HCN2 | 0.016682 |
| ATP2B2 | 0.016653 |
| FEV | 0.016618 |
| FOXO6 | 0.016602 |
| SYNGR4 | 0.016588 |
| NAT8L | 0.016560 |
| FMN2 | 0.016554 |
| GP2 | 0.016551 |
| CENPI | 0.016549 |
| FAM72B | 0.016547 |
| PRDM13 | 0.016546 |
| MAGED4 | 0.016525 |
| SNCB | 0.016521 |
| PACSIN1 | 0.016494 |
| AMPH | 0.016471 |
| IGFBPL1 | 0.016455 |
| SH3GL2 | 0.016426 |
| MLLT11 | 0.016417 |
| DDX3Y | 0.016409 |
| FAM72D | 0.016396 |
| LHFPL4 | 0.016387 |
| CDKN2A | 0.016382 |
| USP9Y | 0.016376 |
| FOXM1 | 0.016374 |
| CCDC154 | 0.016372 |
| UBE2T | 0.016352 |
| SIM1 | 0.016348 |
| E2F8 | 0.016337 |
| KIF20A | 0.016325 |
| ZNF488 | 0.016317 |
| NPTX1 | 0.016305 |
| CBX2 | 0.016293 |
| PAX5 | 0.016275 |
| SLITRK1 | 0.016264 |
| STRC | 0.016263 |
| GPR19 | 0.016259 |
| KIRREL3 | 0.016237 |
| GNG3 | 0.016225 |
| HIST1H2AE | 0.016210 |
| ZIC3 | 0.016189 |
| STIL | 0.016163 |
| CHRNA3 | 0.016138 |
| TMEM249 | 0.016113 |
| CCNA2 | 0.016092 |
| GPR137C | 0.016085 |
| ATRNL1 | 0.016071 |
| HIST1H3C | 0.016057 |
| KLHDC8A | 0.016040 |
| SYCE2 | 0.016009 |
| CELF5 | 0.016007 |
| NKX6-1 | 0.016003 |
| THEG | 0.016001 |
| RRM2 | 0.015992 |
| PLEKHG4B | 0.015989 |
| NPC1L1 | 0.015976 |
| ESCO2 | 0.015971 |
| CBLN1 | 0.015968 |
| PRSS53 | 0.015965 |
| YBX2 | 0.015933 |
| CENPU | 0.015929 |
| CLVS1 | 0.015924 |
| SLC17A4 | 0.015915 |
| CNTD2 | 0.015909 |
| CDCA5 | 0.015902 |
| HIST1H1E | 0.015902 |
| KLK3 | 0.015889 |
| TMEM74 | 0.015877 |
| DOC2A | 0.015870 |
| TMSB15B | 0.015861 |
| HIST1H2AI | 0.015832 |
| HOXB13 | 0.015782 |
| APLP1 | 0.015770 |
| CRIP3 | 0.015760 |
| HIST1H2AG | 0.015756 |
| KIRREL2 | 0.015751 |
| RAB39A | 0.015738 |
| EBF3 | 0.015737 |
| ASF1B | 0.015732 |
| GINS1 | 0.015716 |
| B4GALNT1 | 0.015714 |
| SYN1 | 0.015685 |
| SLC38A3 | 0.015673 |
| EYA2 | 0.015672 |
| MXD3 | 0.015662 |
| PKMYT1 | 0.015650 |
| GRIK2 | 0.015644 |
| CEND1 | 0.015641 |
| PHGR1 | 0.015617 |
| NCAPH | 0.015613 |
| HFM1 | 0.015594 |
| NRCAM | 0.015578 |
| HMMR | 0.015567 |
| GABRG3 | 0.015558 |
| GDF1 | 0.015541 |
| MAGEA12 | 0.015539 |
| GABRA5 | 0.015527 |
| IGDCC3 | 0.015517 |
| NPAS4 | 0.015496 |
| NKPD1 | 0.015485 |
| TMEM151A | 0.015472 |
| KCNH2 | 0.015470 |
| ATP4A | 0.015464 |
| KIF11 | 0.015462 |
| PNMA3 | 0.015454 |
| OLFM3 | 0.015425 |
| MAD2L1 | 0.015401 |
| OPRK1 | 0.015386 |
| CPA5 | 0.015372 |
| NYAPI | 0.015356 |
| SYT10 | 0.015354 |
| MAGEA2B | 0.015353 |
| CDC6 | 0.015345 |
| PLK1 | 0.015342 |
| MYO18B | 0.015337 |
| TLX1 | 0.015324 |
| HIST1H3B | 0.015304 |
| HMX3 | 0.015302 |
| DRP2 | 0.015298 |
| ANKRD18B | 0.015281 |
| BUB1 | 0.015275 |
| KLHL14 | 0.015250 |
| TYMS | 0.015248 |
| SOX2 | 0.015241 |
| SLC26A10 | 0.015233 |
| TCEAL5 | 0.015226 |
| C1orf61 | 0.015223 |
| EME1 | 0.015185 |
| MAPK8IP2 | 0.015182 |
| TAC3 | 0.015180 |
| PDE11A | 0.015172 |
| ISX | 0.015169 |
| ADAM11 | 0.015160 |
| ZWINT | 0.015159 |
| CDKN2C | 0.015159 |
| TCP11X2 | 0.015157 |
| TNFRSF25 | 0.015140 |
| UBE2S | 0.015139 |
| CRMP1 | 0.015138 |
| SYP | 0.015128 |
| CECR6 | 0.015125 |
| ZNF367 | 0.015119 |
| POTEF | 0.015077 |
| TBR1 | 0.015077 |
| DMBX1 | 0.015076 |
| SLC18A3 | 0.015044 |
| COX6B2 | 0.015044 |
| DNAJC12 | 0.015041 |
| DNA2 | 0.015022 |
| OR2B6 | 0.015011 |
| TIGD3 | 0.015001 |
| CDC25A | 0.014976 |
| ADGRB2 | 0.014968 |
| EML6 | 0.014960 |
| ARHGAP11A | 0.014953 |
| IGFL4 | 0.014948 |
| REEP2 | 0.014947 |
| PSAT1 | 0.014928 |
| NHLH1 | 0.014921 |
| NLRP11 | 0.014916 |
| YJEFN3 | 0.014889 |
| KCNMB2 | 0.014882 |
| MEX3B | 0.014878 |
| TMEFF2 | 0.014867 |
| LMO1 | 0.014865 |
| ARHGEF39 | 0.014858 |
| MAPT | 0.014855 |
| RGS8 | 0.014836 |
| CERS1 | 0.014825 |
| FGF5 | 0.014810 |
| GNAO1 | 0.014806 |
| GABRA3 | 0.014800 |
| SLC9A3 | 0.014799 |
| ZNF730 | 0.014788 |
| NKX3-2 | 0.014786 |
| UTY | 0.014783 |
| TET1 | 0.014779 |
| HIST2H3A | 0.014756 |
| CDH22 | 0.014754 |
| ANKRD34B | 0.014752 |
| HIST2H3C | 0.014740 |
| NEUROD2 | 0.014740 |
| ADCY1 | 0.014728 |
| ATAD5 | 0.014706 |
| SIX1 | 0.014683 |
| HIST1H2BF | 0.014669 |
| KSR2 | 0.014667 |
| CENPK | 0.014658 |
| HIST1H1B | 0.014653 |
| OIP5 | 0.014621 |
| ANKRD18A | 0.014609 |
| SMC1B | 0.014604 |
| TRIM46 | 0.014603 |
| KNTC1 | 0.014598 |
| HIST1H2AM | 0.014593 |
| GLYATL2 | 0.014590 |
| KCNN1 | 0.014586 |
| PODXL2 | 0.014582 |
| HTR3A | 0.014579 |
| TFAP2A | 0.014571 |
| VWC2L | 0.014570 |
| STX1A | 0.014552 |
| RET | 0.014529 |
| KIF23 | 0.014522 |
| NPTX2 | 0.014521 |
| ENTPD8 | 0.014495 |
| CDT1 | 0.014480 |
| OTX2 | 0.014469 |
| TEX101 | 0.014443 |
| CTXN2 | 0.014440 |
| SST | 0.014418 |
| CDK1 | 0.014404 |
| PDX1 | 0.014398 |
| CCNE1 | 0.014362 |
| GREB1 | 0.014341 |
| WASF1 | 0.014334 |
| FAM171A2 | 0.014323 |
| CSAG1 | 0.014318 |
| ASB15 | 0.014312 |
| HIST1H1D | 0.014309 |
| FRMPD1 | 0.014309 |
| PTTG1 | 0.014291 |
| XKR5 | 0.014281 |
| IGSF9 | 0.014281 |
| TLX3 | 0.014273 |
| ADAMTS14 | 0.014271 |
| TRIM36 | 0.014266 |
| C20orf203 | 0.014239 |
| DAND5 | 0.014234 |
| CCER2 | 0.014217 |
| NROB1 | 0.014188 |
| SLC16A8 | 0.014187 |
| KISS1R | 0.014181 |
| USH2A | 0.014168 |
| FOXE3 | 0.014160 |
| NLGN4Y | 0.014159 |
| SV2A | 0.014155 |
| ALX4 | 0.014153 |
| SBK1 | 0.014141 |
| VIL1 | 0.014139 |
| ATP6V1G2 | 0.014136 |
| GABRB3 | 0.014131 |
| ARHGDIG | 0.014124 |
| ARHGAP11B | 0.014121 |
| FRMD5 | 0.014118 |
| CHRNA5 | 0.014110 |
| AGMO | 0.014109 |
| LIN28B | 0.014063 |
| CHTF18 | 0.014062 |
| ZFY | 0.014024 |
| CCNB1 | 0.014023 |
| CDCA7 | 0.014014 |
| HOXA11 | 0.013992 |
| FANCB | 0.013990 |
| C2orf70 | 0.013969 |
| AGBL4 | 0.013950 |
| SLC24A2 | 0.013940 |
| CYP2D7 | 0.013939 |
| HIST1H3D | 0.013938 |
| PRR36 | 0.013926 |
| PITX1 | 0.013924 |
| C1QL4 | 0.013921 |
| GRK1 | 0.013901 |
| UGT2A1 | 0.013896 |
| CSMD2 | 0.013895 |
| GPR158 | 0.013886 |
| DNER | 0.013879 |
| USH1C | 0.013842 |
| ENTPD2 | 0.013836 |
| SYT6 | 0.013836 |
| RPS6KL1 | 0.013821 |
| GNB3 | 0.013817 |
| AKR7L | 0.013816 |
| MGAT4C | 0.013814 |
| FOXB1 | 0.013802 |
| PDZD4 | 0.013798 |
| STMN1 | 0.013794 |
| HIST1H2BJ | 0.013793 |
| CEP55 | 0.013789 |
| RAD51AP1 | 0.013770 |
| MMP16 | 0.013741 |
| CPT1C | 0.013738 |
| ORC1 | 0.013734 |
| NLGN1 | 0.013727 |
| ANLN | 0.013717 |
| HIST1H2BL | 0.013714 |
| RPS4Y1 | 0.013697 |
| LHX3 | 0.013689 |
| MT3 | 0.013687 |
| SCG5 | 0.013679 |
| C2CD4C | 0.013667 |
| CHRM4 | 0.013653 |
| RXFP3 | 0.013637 |
| EMILIN3 | 0.013633 |
| ASPDH | 0.013625 |
| CDC7 | 0.013619 |
| GPR173 | 0.013616 |
| DNMT3B | 0.013610 |
| RNF157 | 0.013590 |
| PAX3 | 0.013582 |
| FAM83D | 0.013573 |
| VAX2 | 0.013565 |
| PLEKHH1 | 0.013557 |
| FAM196A | 0.013556 |
| RAB3C | 0.013551 |
| SLC5A12 | 0.013550 |
| TP53TG3 | 0.013542 |
| RAB39B | 0.013537 |
| SLC30A10 | 0.013517 |
| TMEM196 | 0.013515 |
| MAGED4B | 0.013482 |
| PARPBP | 0.013463 |
| DTL | 0.013458 |
| GNRH2 | 0.013440 |
| ACYP1 | 0.013439 |
| C3orf67 | 0.013429 |
| AK5 | 0.013429 |
| HIST3H2BB | 0.013422 |
| GAL3ST3 | 0.013390 |
| SLC8A2 | 0.013384 |
| MEP1B | 0.013381 |
| B3GAT1 | 0.013376 |
| NXF2B | 0.013342 |
| MTMR7 | 0.013340 |
| KCNH3 | 0.013335 |
| PLK4 | 0.013332 |
| SLC6A15 | 0.013319 |
| GRIN2C | 0.013314 |
| RASSF6 | 0.013306 |
| MND1 | 0.013303 |
| IGSF5 | 0.013288 |
| BSND | 0.013285 |
| GCK | 0.013279 |
| RFC4 | 0.013268 |
| GNGT1 | 0.013261 |
| FSIP2 | 0.013256 |
| EIF1AY | 0.013252 |
| MAP1B | 0.013251 |
| NOVA1 | 0.013230 |
| DISP2 | 0.013227 |
| HIST1H4I | 0.013222 |
| DIRAS1 | 0.013211 |
| KIFC2 | 0.013208 |
| POLE2 | 0.013208 |
| C19orf57 | 0.013203 |
| FZD9 | 0.013199 |
| CA10 | 0.013198 |
| STRA6 | 0.013192 |
| ZGRF1 | 0.013191 |
| IGSF9B | 0.013184 |
| BRICD5 | 0.013176 |
| ALLC | 0.013172 |
| PTPRH | 0.013169 |
| DDX11 | 0.013154 |
| KIAA2022 | 0.013129 |
| PSRC1 | 0.013119 |
| TFR2 | 0.013092 |
| AIRE | 0.013085 |
| MARK1 | 0.013078 |
| FANCI | 0.013078 |
| POTEE | 0.013071 |
| FAM222A | 0.013070 |
| COL11A2 | 0.013067 |
| HIST1H3G | 0.013056 |
| PABPC1L | 0.013050 |
| PDXP | 0.013043 |
| HOXD8 | 0.013026 |
| GSC | 0.013020 |
| EVX2 | 0.013015 |
| C5orf34 | 0.012998 |
| ZNF726 | 0.012991 |
| CCDC168 | 0.012990 |
| LYPD6 | 0.012943 |
| MYBL1 | 0.012928 |
| IL17REL | 0.012927 |
| LINGO2 | 0.012927 |
| SLC7A9 | 0.012908 |
| FAM131C | 0.012898 |
| FTCD | 0.012893 |
| TTC6 | 0.012883 |
| NETO1 | 0.012882 |
| E2F1 | 0.012875 |
| ZIC1 | 0.012873 |
| FBXW12 | 0.012861 |
| FOXI3 | 0.012860 |
| GDAP1 | 0.012856 |
| LMNB1 | 0.012849 |
| CABYR | 0.012845 |
| ZCCHC18 | 0.012834 |
| CENPV | 0.012832 |
| BLM | 0.012827 |
| GRIP1 | 0.012821 |
| MY03A | 0.012815 |
| MAGEA1 | 0.012807 |
| FANCA | 0.012803 |
| BMP8A | 0.012797 |
| FAM135B | 0.012787 |
| LRRC53 | 0.012785 |
| HIST1H3F | 0.012784 |
| MUC19 | 0.012774 |
| ATP2A1 | 0.012769 |
| EPHA10 | 0.012767 |
| SLC25A48 | 0.012764 |
| KLK15 | 0.012758 |
| SPAG11B | 0.012751 |
| ATAD2 | 0.012737 |
| ECT2 | 0.012737 |
| RNFT2 | 0.012735 |
| C1orf127 | 0.012733 |
| GRIN3A | 0.012724 |
| ANKRD36 | 0.012723 |
| MYLK2 | 0.012718 |
| HIST3H2A | 0.012718 |
| COL9A1 | 0.012712 |
| ANKRD36C | 0.012707 |
| MTCL1 | 0.012704 |
| CCDC14 | 0.012704 |
| CHST8 | 0.012695 |
| ARTN | 0.012689 |
| RASAL1 | 0.012687 |
| SRCIN1 | 0.012675 |
| TCTE3 | 0.012675 |
| DNAJC6 | 0.012670 |
| RNF165 | 0.012666 |
| LINGO1 | 0.012660 |
| SMPD3 | 0.012658 |
| PNCK | 0.012657 |
| PCK1 | 0.012655 |
| FOXE1 | 0.012652 |
| KCNH4 | 0.012640 |
| PROKR1 | 0.012636 |
| C21orf58 | 0.012635 |
| HCN4 | 0.012634 |
| KIAA1524 | 0.012629 |
| C12orf42 | 0.012624 |
| MCM2 | 0.012621 |
| CA9 | 0.012620 |
| CHEK1 | 0.012614 |
| C4orf50 | 0.012602 |
| CBFA2T2 | 0.012599 |
| HIST1H4C | 0.012583 |
| MURC | 0.012575 |
| TIMELESS | 0.012574 |
| ANKRD13B | 0.012548 |
| BTBD11 | 0.012542 |
| SSTR2 | 0.012541 |
| ZSCAN23 | 0.012539 |
| AURKA | 0.012535 |
| FAM72A | 0.012528 |
| SIM2 | 0.012527 |
| TMEM198 | 0.012506 |
| HPDL | 0.012501 |
| PSD | 0.012499 |
| ADAM22 | 0.012497 |
| PRLHR | 0.012494 |
| CKMT1B | 0.012493 |
| CHRNB4 | 0.012479 |
| HIST1H2AD | 0.012465 |
| FBXO5 | 0.012458 |
| KRT83 | 0.012456 |
| ZNF300 | 0.012452 |
| HIST1H2AL | 0.012446 |
| CPT1B | 0.012444 |
| POTEJ | 0.012432 |
| TCF24 | 0.012430 |
| GRIN1 | 0.012423 |
| PCLO | 0.012404 |
| LRRC26 | 0.012404 |
| SMOC1 | 0.012398 |
| DAPL1 | 0.012396 |
| TRPA1 | 0.012393 |
| KIAA1211 | 0.012367 |
| DBH | 0.012367 |
| NKX2-4 | 0.012363 |
| CEP152 | 0.012358 |
| SLC35F1 | 0.012356 |
| HOXC9 | 0.012353 |
| GSG2 | 0.012345 |
| TONSL | 0.012317 |
| GPR61 | 0.012314 |
| SYNGR3 | 0.012303 |
| PTH2R | 0.012290 |
| NCAPG2 | 0.012273 |
| MMP11 | 0.012263 |
| JPH3 | 0.012249 |
| CACNG5 | 0.012244 |
| GAL | 0.012236 |
| PILRB | 0.012232 |
| VSTM2A | 0.012222 |
| COL7A1 | 0.012221 |
| CCNF | 0.012217 |
| POU6F2 | 0.012205 |
| TAL2 | 0.012205 |
| ZFP69B | 0.012200 |
| SYT16 | 0.012198 |
| ANKLE1 | 0.012192 |
| MCM4 | 0.012188 |
| DONSON | 0.012188 |
| CDH24 | 0.012155 |
| TP73 | 0.012145 |
| RPRML | 0.012141 |
| FBXO41 | 0.012126 |
| KIAA1024 | 0.012126 |
| ZNF519 | 0.012123 |
| SYT7 | 0.012122 |
| MANEAL | 0.012117 |
| SIX4 | 0.012077 |
| MYEF2 | 0.012068 |
| KCNJ3 | 0.012059 |
| HRK | 0.012055 |
| GOLGA8A | 0.012053 |
| CLEC18B | 0.012046 |
| CPA2 | 0.012039 |
| CLSTN2 | 0.012030 |
| MAP6D1 | 0.012025 |
| SPTBN2 | 0.012014 |
| LRRC24 | 0.011998 |
| ERAS | 0.011996 |
| POU3F3 | 0.011995 |
| ASTN1 | 0.011984 |
| UQCRHL | 0.011961 |
| ASTN2 | 0.011953 |
| HIST2H2AC | 0.011948 |
| AUNIP | 0.011945 |
| HOXC6 | 0.011942 |
| RNF182 | 0.011938 |
| ATP4B | 0.011926 |
| MAMDC4 | 0.011911 |
| FAM166A | 0.011893 |
| PROZ | 0.011881 |
| FABP7 | 0.011881 |
| TRIP13 | 0.011871 |
| SCAMP5 | 0.011871 |
| SMEM24 | 0.011865 |
| VWDE | 0.011848 |
| DSCC1 | 0.011838 |
| PRR11 | 0.011835 |
| RNF186 | 0.011834 |
| ANKRD36B | 0.011833 |
| HMGB2 | 0.011815 |
| AKR7A3 | 0.011802 |
| FAM171B | 0.011796 |
| HYLS1 | 0.011793 |
| CCDC34 | 0.011787 |
| MTBP | 0.011784 |
| PIDD1 | 0.011769 |
| SEC11C | 0.011769 |
| RIBC2 | 0.011763 |
| WDR27 | 0.011760 |
| CDH18 | 0.011754 |
| KCNJ4 | 0.011746 |
| TOX3 | 0.011744 |
| MCF2L2 | 0.011732 |
| GBX2 | 0.011722 |
| PATE4 | 0.011719 |
| PCDH19 | 0.011718 |
| ZNF850 | 0.011711 |
| ADARB2 | 0.011708 |
| SLC29A4 | 0.011707 |
| CUX2 | 0.011692 |
| HIST1H2AH | 0.011690 |
| MAP7D2 | 0.011687 |
| UGT8 | 0.011682 |
| MEST | 0.011677 |
| CHAF1B | 0.011675 |
| TSHR | 0.011672 |
| NXF2 | 0.011657 |
| DNM1 | 0.011655 |
| MAGEA9B | 0.011645 |
| KCNAB3 | 0.011643 |
| CENPM | 0.011639 |
| TAF7L | 0.011620 |
| ARG2 | 0.011583 |
| CDH12 | 0.011580 |
| PSMC3IP | 0.011578 |
| SH2D5 | 0.011575 |
| TRPC5 | 0.011570 |
| PAQR9 | 0.011567 |
| AHSG | 0.011566 |
| KCNH1 | 0.011540 |
| ISM2 | 0.011536 |
| B3GALT1 | 0.011531 |
| AVPR1B | 0.011525 |
| DUSP26 | 0.011523 |
| TCP10L | 0.011516 |
| TCF19 | 0.011514 |
| MPHOSPH9 | 0.011488 |
| KIF18A | 0.011487 |
| SLC44A5 | 0.011486 |
| ERC2 | 0.011482 |
| HIST1H4B | 0.011467 |
| PPP1R17 | 0.011443 |
| SLC12A1 | 0.011434 |
| IRX4 | 0.011427 |
| MRAP2 | 0.011425 |
| ZNF793 | 0.011415 |
| PNMA5 | 0.011413 |
| GNAZ | 0.011407 |
| KRTAP5-1 | 0.011407 |
| RAB9B | 0.011406 |
| CXXC4 | 0.011405 |
| UMODL1 | 0.011404 |
| RAD54B | 0.011400 |
| LYG1 | 0.011398 |
| ZFHX4 | 0.011390 |
| HIST1H4J | 0.011387 |
| IBSP | 0.011380 |
| CA8 | 0.011352 |
| FAM161A | 0.011349 |
| ATP6V1C2 | 0.011346 |
| PRPH | 0.011342 |
| GABRR2 | 0.011342 |
| CADM2 | 0.011341 |
| KIF24 | 0.011337 |
| CKMT1A | 0.011334 |
| HOXD9 | 0.011311 |
| NPIPA2 | 0.011308 |
| STMN3 | 0.011297 |
| MYADML2 | 0.011281 |
| ASNS | 0.011277 |
| HOXB3 | 0.011268 |
| LONRF2 | 0.011248 |
| CCDC129 | 0.011246 |
| DGKI | 0.011244 |
| PAQR6 | 0.011218 |
| ANKS1B | 0.011202 |
| EMX2 | 0.011200 |
| ANGPTL3 | 0.011194 |
| GPT2 | 0.011184 |
| CORT | 0.011179 |
| RACGAP1 | 0.011176 |
| MCM8 | 0.011168 |
| CDHR1 | 0.011166 |
| RAC3 | 0.011161 |
| NPPB | 0.011157 |
| TENM3 | 0.011146 |
| HIST1H4D | 0.011130 |
| EPHB1 | 0.011125 |
| RPP14 | 0.011123 |
| GALR1 | 0.011122 |
| C2orf82 | 0.011121 |
| TTYH1 | 0.011112 |
| DACH2 | 0.011103 |
| MEIOB | 0.011101 |
| SPDYA | 0.011078 |
| PATE2 | 0.011075 |
| HOXC10 | 0.011074 |
| SLC38A8 | 0.011072 |
| SAPCD1 | 0.011069 |
| TTC9B | 0.011066 |
| CYP2D6 | 0.011063 |
| CCDC136 | 0.011060 |
| GRIK1 | 0.011045 |
| CTXN1 | 0.011043 |
| POLE | 0.011040 |
| DCLK3 | 0.011037 |
| JAKMIP1 | 0.011031 |
| DYNC1I1 | 0.011030 |
| AQP12B | 0.011022 |
| CALY | 0.011018 |
| KIAA0895L | 0.011010 |
| CGA | 0.011008 |
| ZNF556 | 0.011008 |
| PCSK2 | 0.011006 |
| TDRD12 | 0.011000 |
| CDK3 | 0.010994 |
| SV2B | 0.010988 |
| TTLL7 | 0.010988 |
| B3GAT2 | 0.010987 |
| ZP1 | 0.010964 |
| PFN2 | 0.010954 |
| WDR76 | 0.010952 |
| PSD2 | 0.010949 |
| ZIC4 | 0.010949 |
| AVP | 0.010949 |
| KLF1 | 0.010945 |
| FIGNL1 | 0.010930 |
| CTSV | 0.010930 |
| PPP2R3B | 0.010919 |
| ICA1L | 0.010919 |
| TRIM72 | 0.010917 |
| KCNB1 | 0.010904 |
| TAS2R19 | 0.010900 |
| ESPN | 0.010895 |
| BRCA1 | 0.010890 |
| CKAP2 | 0.010883 |
| MGAM2 | 0.010881 |
| C11orf87 | 0.010879 |
| ZNF233 | 0.010874 |
| TRO | 0.010859 |
| HIST1H3J | 0.010858 |
| DBF4B | 0.010858 |
| NPTXR | 0.010857 |
| TLL2 | 0.010852 |
| SSX2IP | 0.010839 |
| GPX2 | 0.010837 |
| NPPC | 0.010827 |
| IGF2BP3 | 0.010827 |
| GINS4 | 0.010821 |
| UTS2R | 0.010806 |
| KIF19 | 0.010804 |
| TMPRSS5 | 0.010804 |
| TSPEAR | 0.010803 |
| WDR90 | 0.010795 |
| ZNF93 | 0.010785 |
| TXNDC16 | 0.010784 |
| CPA4 | 0.010776 |
| FAM24B | 0.010775 |
| ASB16 | 0.010760 |
| SMIM17 | 0.010737 |
| USP49 | 0.010729 |
| HPX | 0.010725 |
| RMI2 | 0.010714 |
| PPFIA2 | 0.010712 |
| BARHL1 | 0.010711 |
| TFF3 | 0.010703 |
| TTC36 | 0.010690 |
| MMP12 | 0.010689 |
| ANKRD23 | 0.010687 |
| WDHD1 | 0.010687 |
| MIOX | 0.010683 |
| C14orf39 | 0.010682 |
| MMD2 | 0.010681 |
| CCDC74B | 0.010662 |
| MAPK8IP1 | 0.010658 |
| FRMPD3 | 0.010652 |
| CNPY1 | 0.010645 |
| 43713 | 0.010640 |
| SYT2 | 0.010638 |
| LIN28A | 0.010622 |
| PGBD5 | 0.010620 |
| GABRR1 | 0.010612 |
| DAB1 | 0.010610 |
| C19orf48 | 0.010605 |
| ARPP21 | 0.010593 |
| HIST1H2AK | 0.010592 |
| MUC6 | 0.010588 |
| GMNN | 0.010584 |
| DPP10 | 0.010576 |
| L3MBTL1 | 0.010574 |
| LRRIQ4 | 0.010574 |
| GDNF | 0.010566 |
| DAZ1 | 0.010565 |
| DNAH14 | 0.010556 |
| ECEL1 | 0.010556 |
| EN1 | 0.010556 |
| KRT75 | 0.010555 |
| COL20A1 | 0.010554 |
| SUGP2 | 0.010554 |
| NPW | 0.010553 |
| C20orf144 | 0.010537 |
| GNRHR | 0.010536 |
| RGS4 | 0.010535 |
| SI | 0.010530 |
| CDH9 | 0.010527 |
| SKP2 | 0.010527 |
| TP53TG3D | 0.010525 |
| BMP8B | 0.010523 |
| SLC12A5 | 0.010521 |
| SRRM5 | 0.010520 |
| GPR26 | 0.010518 |
| CACNG2 | 0.010517 |
| TRPM5 | 0.010507 |
| LTB4R2 | 0.010502 |
| CUZD1 | 0.010495 |
| KCND2 | 0.010491 |
| TP53TG3B | 0.010484 |
| KCNG3 | 0.010478 |
| SS18L1 | 0.010474 |
| TMEM132A | 0.010472 |
| FZD3 | 0.010472 |
| RAD51 | 0.010470 |
| PLEKHG5 | 0.010466 |
| SEC61A2 | 0.010463 |
| STAG3 | 0.010459 |
| GLYATL3 | 0.010456 |
| ST6GAL2 | 0.010456 |
| ASPHD1 | 0.010453 |
| PRR7 | 0.010452 |
| SPRED3 | 0.010445 |
| HIST1H2BE | 0.010444 |
| GABRG1 | 0.010442 |
| SPAG11A | 0.010436 |
| SNCAIP | 0.010435 |
| PAX9 | 0.010425 |
| DCAF12L2 | 0.010424 |
| CENPW | 0.010399 |
| RHBDL1 | 0.010395 |
| MMP26 | 0.010392 |
| PHOX2A | 0.010385 |
| OTOF | 0.010381 |
| MAGEA10 | 0.010381 |
| PIANP | 0.010380 |
| SCN5A | 0.010375 |
| LYG2 | 0.010368 |
| EVX1 | 0.010368 |
| SMC2 | 0.010368 |
| PANO1 | 0.010353 |
| ANKRD20A4 | 0.010352 |
| PI15 | 0.010349 |
| ACAP3 | 0.010346 |
| ZNF878 | 0.010340 |
| IGSF1 | 0.010332 |
| ATAT1 | 0.010330 |
| PRAC2 | 0.010330 |
| RHPN1 | 0.010305 |
| HSF4 | 0.010303 |
| PRKCG | 0.010302 |
| TMEM169 | 0.010300 |
| NFASC | 0.010300 |
| CALML6 | 0.010295 |
| PTGES3L | 0.010286 |
| ALB | 0.010286 |
| LYPD1 | 0.010285 |
| LIG1 | 0.010277 |
| C3orf30 | 0.010267 |
| WEE2 | 0.010262 |
| ADGRB1 | 0.010260 |
| GAS2L3 | 0.010250 |
| CHRNA9 | 0.010249 |
| GCGR | 0.010239 |
| NRTN | 0.010236 |
| DND1 | 0.010224 |
| SPDL1 | 0.010216 |
| CATSPER2 | 0.010210 |
| MYH7B | 0.010208 |
| KIAA0907 | 0.010206 |
| ESRRG | 0.010201 |
| SPSB4 | 0.010200 |
| TAS2R10 | 0.010199 |
| CENPH | 0.010196 |
| ZNF280B | 0.010195 |
| SPIN4 | 0.010194 |
| ZC3HAV1L | 0.010194 |
| SLC22A7 | 0.010183 |
| DCLK2 | 0.010182 |
| RNASEH2A | 0.010180 |
| LRFN5 | 0.010164 |
| NPHS1 | 0.010154 |
| PAQR4 | 0.010153 |
| DNAJC22 | 0.010149 |
| IL17RB | 0.010141 |
| SEC31B | 0.010140 |
| LCN12 | 0.010139 |
| FBXL16 | 0.010137 |
| CHD7 | 0.010132 |
| UBL4B | 0.010122 |
| CLEC18A | 0.010120 |
| ENHO | 0.010114 |
| GEN1 | 0.010111 |
| IGSF11 | 0.010111 |
| LHX4 | 0.010094 |
| FANCL | 0.010092 |
| TRAIP | 0.010061 |
| PKIB | 0.010059 |
| SLC35F4 | 0.010052 |
| FGF12 | 0.010050 |
| TRIM45 | 0.010046 |
| C10orf90 | 0.010037 |
| MYO19 | 0.010031 |
| MMS22L | 0.010029 |
| SYCP2 | 0.010022 |
| LHX1 | 0.010022 |
| TEX22 | 0.010021 |
| GPR63 | 0.010018 |
| SLC36A4 | 0.010016 |
| HS6ST3 | 0.010012 |
| MESP1 | 0.010010 |
| OVOL3 | 0.010006 |
| LHX5 | 0.010003 |
| GPR142 | 0.010002 |
| RASL10B | 0.009994 |
| OXCT2 | 0.009992 |
| MARCKSL1 | 0.009992 |
| CCDC18 | 0.009991 |
| FANCG | 0.009991 |
| ABCA5 | 0.009986 |
| FSD1L | 0.009985 |
| CST1 | 0.009982 |
| CALN1 | 0.009973 |
| ONECUT1 | 0.009971 |
| GOLGA6L10 | 0.009970 |
| HOXB2 | 0.009968 |
| CEP72 | 0.009967 |
| ZNF334 | 0.009965 |
| TENM4 | 0.009965 |
| HIST4H4 | 0.009948 |
| KCNMB3 | 0.009932 |
| ZNF713 | 0.009924 |
| HIST1H2AJ | 0.009921 |
| MASP2 | 0.009917 |
| KHDC1 | 0.009916 |
| CEP97 | 0.009911 |
| KCNIP2 | 0.009911 |
| KCNJ9 | 0.009887 |
| CCDC15 | 0.009881 |
| CHAF1A | 0.009879 |
| LRRC66 | 0.009866 |
| SATL1 | 0.009865 |
| CEP295 | 0.009861 |
| PRSS50 | 0.009859 |
| TNFRSF13C | 0.009859 |
| IQCC | 0.009858 |
| NCAPD3 | 0.009858 |
| TPH2 | 0.009857 |
| SCX | 0.009853 |
| PAK3 | 0.009851 |
| SALL4 | 0.009845 |
| RBFOX3 | 0.009841 |
| EME2 | 0.009838 |
| OLFM4 | 0.009837 |
| TUBA3D | 0.009834 |
| ZNF625 | 0.009827 |
| IL23A | 0.009824 |
| ASIC3 | 0.009818 |
| MSI2 | 0.009816 |
| NANOS1 | 0.009808 |
| NEUROG1 | 0.009802 |
| NKX2-5 | 0.009802 |
| CPLX1 | 0.009802 |
| C17orf53 | 0.009802 |
| GKAP1 | 0.009792 |
| THOC1 | 0.009782 |
| POU4F2 | 0.009781 |
| COL11A1 | 0.009778 |
| TMPO | 0.009777 |
| SPIN2A | 0.009774 |
| GPR83 | 0.009773 |
| RFC3 | 0.009772 |
| DTX1 | 0.009766 |
| SASS6 | 0.009752 |
| XRCC3 | 0.009750 |
| C1orf112 | 0.009746 |
| ZNF681 | 0.009742 |
| F12 | 0.009727 |
| DBF4 | 0.009717 |
| PDK1 | 0.009698 |
| RELN | 0.009698 |
| CCNL2 | 0.009690 |
| ZNF891 | 0.009685 |
| GREB1L | 0.009681 |
| LRRC14B | 0.009676 |
| SLC4A10 | 0.009676 |
| SLC4A3 | 0.009675 |
| SEMA6C | 0.009673 |
| PGAP1 | 0.009669 |
| ZGLP1 | 0.009665 |
| NT5M | 0.009664 |
| WFKKN1 | 0.009663 |
| MTNRIB | 0.009660 |
| TUBB8 | 0.009660 |
| CLEC18C | 0.009659 |
| NPAS3 | 0.009659 |
| MAPK12 | 0.009657 |
| VASH2 | 0.009651 |
| RCCD1 | 0.009638 |
| FEN1 | 0.009635 |
| HIST1H3A | 0.009629 |
| CGREF1 | 0.009629 |
| CACNA1G | 0.009629 |
| C1QTNF4 | 0.009627 |
| ANKRD20A1 | 0.009626 |
| BARD1 | 0.009624 |
| C18orf54 | 0.009621 |
| CCDC28B | 0.009619 |
| LY6G5B | 0.009616 |
| SPTB | 0.009610 |
| CACNA2D1 | 0.009602 |
| CFHR3 | 0.009600 |
| HAND2 | 0.009599 |
| PAXBP1 | 0.009596 |
| DNASE1 | 0.009586 |
| SERPINB5 | 0.009577 |
| CD24 | 0.009576 |
| C19orf71 | 0.009575 |
| KCNG1 | 0.009574 |
| SCRT2 | 0.009571 |
| VSTM2B | 0.009556 |
| CNKSR3 | 0.009551 |
| FAM227A | 0.009546 |
| ANKRD61 | 0.009541 |
| MYCN | 0.009540 |
| MSH5 | 0.009538 |
| RFX4 | 0.009535 |
| TMEM200C | 0.009532 |
| ADCY10 | 0.009528 |
| OTOP3 | 0.009526 |
| ABCC5 | 0.009526 |
| RSRP1 | 0.009523 |
| TAAR1 | 0.009521 |
| SUV39H2 | 0.009521 |
| COL9A3 | 0.009520 |
| ZNF714 | 0.009512 |
| ZFP30 | 0.009510 |
| CELF6 | 0.009508 |
| PPAT | 0.009492 |
| EFNA3 | 0.009486 |
| ENO2 | 0.009463 |
| MYCL | 0.009461 |
| ADAM23 | 0.009460 |
| CASA | 0.009460 |
| MCM7 | 0.009458 |
| C9orf40 | 0.009456 |
| SMC4 | 0.009454 |
| RNASE10 | 0.009436 |
| TCP11X1 | 0.009436 |
| TACC3 | 0.009434 |
| KIAA1456 | 0.009434 |
| KCTD19 | 0.009432 |
| SLC4A8 | 0.009431 |
| PRDM15 | 0.009429 |
| BGLAP | 0.009422 |
| ARHGAP36 | 0.009420 |
| ZNF692 | 0.009420 |
| CTRC | 0.009417 |
| SLC25A27 | 0.009401 |
| ZNF705E | 0.009388 |
| AQP11 | 0.009388 |
| CALML5 | 0.009373 |
| TEX29 | 0.009370 |
| LRRC10 | 0.009366 |
| FOXH1 | 0.009362 |
| SUMO4 | 0.009359 |
| POU3F1 | 0.009344 |
| ILDR2 | 0.009340 |
| PHGDH | 0.009337 |
| FAM81A | 0.009333 |
| EN2 | 0.009331 |
| MYOD1 | 0.009328 |
| GRM7 | 0.009322 |
| CEP78 | 0.009322 |
| CDX2 | 0.009322 |
| CEP85 | 0.009320 |
| MYBPC1 | 0.009320 |
| DRD4 | 0.009309 |
| GOLGA6A | 0.009303 |
| CDHR2 | 0.009301 |
| FAM84A | 0.009300 |
| GNRH1 | 0.009296 |
| FAM169A | 0.009294 |
| TMPRSS9 | 0.009291 |
| KIAA0319 | 0.009290 |
| ROPN1B | 0.009283 |
| SCNN1D | 0.009273 |
| MC4R | 0.009272 |
| PTBP2 | 0.009263 |
| NKX6-3 | 0.009261 |
| BRS3 | 0.009252 |
| NPIPA7 | 0.009247 |
| NPM2 | 0.009242 |
| ERICH5 | 0.009236 |
| INCENP | 0.009233 |
| HIST1H4A | 0.009232 |
| RAB40AL | 0.009227 |
| GLDC | 0.009221 |
| PASK | 0.009218 |
| APOF | 0.009207 |
| ZNF594 | 0.009204 |
| FRMD1 | 0.009202 |
| MB | 0.009201 |
| SLC39A5 | 0.009196 |
| UNC93A | 0.009196 |
| HOXB1 | 0.009183 |
| TOPBP1 | 0.009181 |
| EN03 | 0.009180 |
| GUCA1B | 0.009168 |
| ZBTB12 | 0.009168 |
| RIMKLA | 0.009166 |
| NWD2 | 0.009162 |
| GOLGA6L9 | 0.009157 |
| HIST1H3E | 0.009157 |
| HAUS5 | 0.009150 |
| SPERT | 0.009149 |
| SMKR1 | 0.009145 |
| KLHL1 | 0.009143 |
| CENPL | 0.009143 |
| PCNA | 0.009140 |
| HIST2H4A | 0.009139 |
| ZNF782 | 0.009138 |
| PMFBP1 | 0.009136 |
| SOX21 | 0.009135 |
| SDK2 | 0.009131 |
| PDZK1 | 0.009129 |
| MIA | 0.009127 |
| CDCA4 | 0.009126 |
| DSCAML1 | 0.009124 |
| HIST1H3H | 0.009119 |
| NEFL | 0.009118 |
| MAST2 | 0.009118 |
| PROC | 0.009116 |
| ZNF253 | 0.009107 |
| MADCAM1 | 0.009103 |
| UCK2 | 0.009102 |
| HIST1H2AB | 0.009101 |
| HIST2H3D | 0.009099 |
| FRMPD2 | 0.009082 |
| PLGLB1 | 0.009081 |
| CLTCL1 | 0.009079 |
| HLTF | 0.009076 |
| A1CF | 0.009066 |
| WBSCR28 | 0.009065 |
| KCNS2 | 0.009063 |
| RIC3 | 0.009054 |
| MTFR2 | 0.009053 |
| IL11 | 0.009048 |
| PIWIL1 | 0.009044 |
| SNTG2 | 0.009039 |
| PROX2 | 0.009032 |
| QRICH2 | 0.009027 |
| CEP41 | 0.009026 |
| C1QL2 | 0.009025 |
| CRHR2 | 0.009024 |
| NOXRED1 | 0.009023 |
| KLHL23 | 0.009023 |
| MSH4 | 0.009015 |
| TAS2R31 | 0.009014 |
| CCDC74A | 0.009013 |
| CENPO | 0.009013 |
| SMCO2 | 0.009007 |
| MOV10L1 | 0.009007 |
| KATNAL2 | 0.009004 |
| UNC5D | 0.009003 |
| HOXD12 | 0.009003 |
| PSMA2 | 0.009002 |
| SLCO6A1 | 0.008994 |
| SYNJ2 | 0.008991 |
| SLC7A11 | 0.008989 |
| RFC5 | 0.008988 |
| MCF2L | 0.008986 |
| SAP25 | 0.008979 |
| ANO4 | 0.008976 |
| WNT16 | 0.008976 |
| SOX4 | 0.008974 |
| OR13A1 | 0.008972 |
| PAX4 | 0.008972 |
| KIF3C | 0.008968 |
| AGXT | 0.008968 |
| SPTSSB | 0.008967 |
| POMK | 0.008963 |
| H2AFX | 0.008949 |
| IGFBP2 | 0.008941 |
| STRIP2 | 0.008941 |
| ALX1 | 0.008934 |
| CIART | 0.008933 |
| C9orf172 | 0.008931 |
| ZNF708 | 0.008928 |
| PRR18 | 0.008921 |
| RAB3A | 0.008921 |
| KCNF1 | 0.008920 |
| CDKN2D | 0.008913 |
| HOXC4 | 0.008911 |
| MAPK8IP3 | 0.008909 |
| CKS2 | 0.008905 |
| FOXL2NB | 0.008897 |
| TTC14 | 0.008892 |
| ATP8A2 | 0.008891 |
| BEGAIN | 0.008890 |
| PCBP3 | 0.008888 |
| ZNF732 | 0.008887 |
| SPOCK3 | 0.008884 |
| NUP62CL | 0.008883 |
| ZNF705G | 0.008881 |
| HIST1H3I | 0.008880 |
| ADAMTS20 | 0.008877 |
| NETO2 | 0.008876 |
| CPNE7 | 0.008869 |
| RPS6KA6 | 0.008869 |
| SHCBP1 | 0.008865 |
| KPNA2 | 0.008858 |
| TIGD1 | 0.008851 |
| ATXN7L2 | 0.008849 |
| JAKMIP3 | 0.008842 |
| ZNF107 | 0.008841 |
| PEG10 | 0.008841 |
| HOXB7 | 0.008839 |
| UROC1 | 0.008838 |
| SYN2 | 0.008835 |
| TMEM184A | 0.008829 |
| FANCD2 | 0.008814 |
| PNMA6A | 0.008813 |
| HIST1H2BM | 0.008813 |
| LCT | 0.008811 |
| MAD2L2 | 0.008808 |
| CPE | 0.008808 |
| CSRNP3 | 0.008805 |
| CCDC110 | 0.008800 |
| CBY3 | 0.008798 |
| PPP1R3E | 0.008797 |
| VAT1L | 0.008796 |
| NAPB | 0.008782 |
| FGD1 | 0.008779 |
| CHRM5 | 0.008776 |
| ZNF483 | 0.008775 |
| SRD5A1 | 0.008775 |
| GOLGA7B | 0.008773 |
| FAM216A | 0.008770 |
| SKOR1 | 0.008757 |
| AK4 | 0.008757 |
| RMI1 | 0.008756 |
| NCR3LG1 | 0.008750 |
| JAKMIP2 | 0.008749 |
| MYL7 | 0.008747 |
| NECAB2 | 0.008739 |
| MAGEA11 | 0.008737 |
| TBC1D3B | 0.008736 |
| YPEL1 | 0.008731 |
| CNFN | 0.008718 |
| MAGEC1 | 0.008709 |
| HIST2H2AB | 0.008702 |
| CASP8AP2 | 0.008693 |
| FGF21 | 0.008688 |
| PHF6 | 0.008686 |
| PRB3 | 0.008686 |
| SKIDA1 | 0.008681 |
| C3orf22 | 0.008680 |
| SAP30 | 0.008673 |
| MEF2B | 0.008673 |
| CEP192 | 0.008672 |
| MSH2 | 0.008669 |
| ABLIM2 | 0.008669 |
| ZNF157 | 0.008667 |
| PRPF39 | 0.008662 |
| ZNF736 | 0.008658 |
| AGPAT5 | 0.008653 |
| LRRTM2 | 0.008653 |
| THRSP | 0.008650 |
| KNSTRN | 0.008644 |
| TEX19 | 0.008640 |
| IZUMO4 | 0.008636 |
| TEX30 | 0.008634 |
| CENPN | 0.008630 |
| CALHM1 | 0.008629 |
| EFCAB8 | 0.008624 |
| ARL17A | 0.008623 |
| MAGEC2 | 0.008621 |
| BIK | 0.008619 |
| MTUS2 | 0.008617 |
| NASP | 0.008612 |
| PRRT2 | 0.008612 |
| KIF20B | 0.008609 |
| BRCA2 | 0.008607 |
| ZNF221 | 0.008603 |
| GABRG2 | 0.008601 |
| TBC1D31 | 0.008598 |
| HMGB3 | 0.008598 |
| AMIG03 | 0.008593 |
| CENPJ | 0.008591 |
| SPPL2B | 0.008584 |
| CTHRC1 | 0.008579 |
| PRUNE2 | 0.008577 |
| DDN | 0.008576 |
| DDX39A | 0.008575 |
| TRIM59 | 0.008574 |
| DCT | 0.008572 |
| LCN8 | 0.008570 |
| PAN2 | 0.008570 |
| APBA2 | 0.008561 |
| CEP131 | 0.008560 |
| PALM2 | 0.008550 |
| C11orf42 | 0.008548 |
| EID2B | 0.008548 |
| MAGEA9 | 0.008547 |
| PANX3 | 0.008545 |
| QSOX2 | 0.008543 |
| GPT | 0.008540 |
| ITGB3BP | 0.008539 |
| ATAD3B | 0.008538 |
| OLIG2 | 0.008537 |
| KCTD8 | 0.008532 |
| RHEBL1 | 0.008530 |
| UGT3A1 | 0.008527 |
| TMCC2 | 0.008526 |
| LRRC75A | 0.008518 |
| ZNF789 | 0.008513 |
| LING03 | 0.008512 |
| HOXC8 | 0.008499 |
| FEZF2 | 0.008497 |
| ZBED8 | 0.008492 |
| HAP1 | 0.008489 |
| PGBD1 | 0.008487 |
| TMSB4Y | 0.008486 |
| FLVCR1 | 0.008481 |
| GUCA1A | 0.008480 |
| WDR88 | 0.008477 |
| POU5F2 | 0.008477 |
| NBPF20 | 0.008474 |
| DTNA | 0.008466 |
| AHI1 | 0.008465 |
| RBP3 | 0.008465 |
| ZC3H8 | 0.008463 |
| VPS37D | 0.008457 |
| SPTBN5 | 0.008456 |
| C1orf94 | 0.008439 |
| AVIL | 0.008437 |
| FAM159B | 0.008435 |
| TBX18 | 0.008427 |
| FEZF1 | 0.008425 |
| MCOLN3 | 0.008425 |
| CYP4F8 | 0.008425 |
| SBK2 | 0.008425 |
| CCP110 | 0.008422 |
| HIST1H2BN | 0.008420 |
| HIST2H2AA4 | 0.008411 |
| CCDC183 | 0.008409 |
| NBEA | 0.008404 |
| FOXL2 | 0.008403 |
| QPCT | 0.008402 |
| CA6 | 0.008398 |
| HMSD | 0.008396 |
| DNMT1 | 0.008396 |
| KCNJ11 | 0.008393 |
| SEMA5B | 0.008380 |
| ABCA12 | 0.008378 |
| SAMD7 | 0.008374 |
| UCN2 | 0.008371 |
| CDKAL1 | 0.008369 |
| SLC35E2 | 0.008369 |
| TAS2R5 | 0.008367 |
| CENPP | 0.008366 |
| EPHA5 | 0.008363 |
| AHSA2 | 0.008352 |
| LHX8 | 0.008350 |
| GDF9 | 0.008346 |
| SPIRE2 | 0.008343 |
| ZNF92 | 0.008339 |
| GGH | 0.008335 |
| DDX53 | 0.008333 |
| DRICH1 | 0.008321 |
| PPFIA3 | 0.008320 |
| SLC10A4 | 0.008318 |
| SOHLH1 | 0.008317 |
| SMIM18 | 0.008314 |
| NRIP3 | 0.008311 |
| TENM1 | 0.008308 |
| SCGB1D1 | 0.008303 |
| DSPP | 0.008303 |
| HIST2H4B | 0.008299 |
| HOOK1 | 0.008298 |
| BOLL | 0.008297 |
| B4GALT6 | 0.008295 |
| CCDC138 | 0.008292 |
| SLC34A3 | 0.008292 |
| NMNAT3 | 0.008292 |
| REM2 | 0.008289 |
| BCL2L10 | 0.008288 |
| PTPRN2 | 0.008281 |
| LDLRAD3 | 0.008275 |
| TWIST1 | 0.008271 |
| BDH1 | 0.008268 |
| LEAP2 | 0.008256 |
| C1QL3 | 0.008253 |
| CHN2 | 0.008249 |
| PKP2 | 0.008239 |
| LMNB2 | 0.008237 |
| ZNF606 | 0.008237 |
| ZNF587 | 0.008235 |
| AGAP4 | 0.008235 |
| CELSR2 | 0.008233 |
| PSIP1 | 0.008229 |
| TBC1D32 | 0.008222 |
| MYB | 0.008217 |
| DNAJB7 | 0.008216 |
| WNT11 | 0.008211 |
| SPATA25 | 0.008208 |
| MATN1 | 0.008203 |
| FAXC | 0.008201 |
| SSUH2 | 0.008191 |
| ZNF84 | 0.008190 |
| PGM2L1 | 0.008189 |
| LRRC39 | 0.008185 |
| PABPN1L | 0.008185 |
| EYA1 | 0.008183 |
| RELL2 | 0.008171 |
| GATS | 0.008169 |
| BRI3BP | 0.008161 |
| GPR27 | 0.008160 |
| NAA40 | 0.008159 |
| CFHR5 | 0.008158 |
| PAX6 | 0.008158 |
| CREBZF | 0.008157 |
| ZNF83 | 0.008157 |
| TRMU | 0.008155 |
| POLA1 | 0.008150 |
| NEMP1 | 0.008150 |
| BSX | 0.008148 |
| ADAM32 | 0.008146 |
| C4orf46 | 0.008146 |
| E2F3 | 0.008145 |
| PKD1 | 0.008145 |
| IL17RD | 0.008138 |
| FOXI1 | 0.008134 |
| PLCB4 | 0.008132 |
| DSP | 0.008131 |
| PROCA1 | 0.008130 |
| BHLHA15 | 0.008130 |
| RBM12B | 0.008116 |
| C8orf44 | 0.008113 |
| FJX1 | 0.008113 |
| TP53AIP1 | 0.008110 |
| ZNF121 | 0.008109 |
| TSACC | 0.008108 |
| LRRD1 | 0.008105 |
| GPR88 | 0.008104 |
| UPF3B | 0.008099 |
| ZNF780B | 0.008099 |
| AGAP6 | 0.008097 |
| CYP2E1 | 0.008086 |
| H2AFY2 | 0.008079 |
| ALDH8A1 | 0.008077 |
| FAM221A | 0.008077 |
| CEP57L1 | 0.008077 |
| NR5A1 | 0.008075 |
| ZNF525 | 0.008071 |
| SPRN | 0.008069 |
| NPIPA1 | 0.008064 |
| WNT8B | 0.008061 |
| TMEM235 | 0.008058 |
| RGAG1 | 0.008048 |
| FOXD2 | 0.008048 |
| ATG9B | 0.008048 |
| F2 | 0.008044 |
| HAPLN1 | 0.008041 |
| GATSL2 | 0.008039 |
| INHA | 0.008037 |
| IMMP1L | 0.008034 |
| PNN | 0.008031 |
| EFNA2 | 0.008029 |
| CEP250 | 0.008025 |
| MAB21L1 | 0.008021 |
| CABP1 | 0.008011 |
| PAX1 | 0.008010 |
| ADAM20 | 0.008008 |
| SPIN3 | 0.008008 |
| CRYGS | 0.008008 |
| EMC9 | 0.008007 |
| ISLR2 | 0.008004 |
| ZFHX2 | 0.008004 |
| HTR6 | 0.008004 |
| OFCC1 | 0.008003 |
| DNAJC9 | 0.008003 |
| C4orf48 | 0.007997 |
| POMC | 0.007995 |
| OCLM | 0.007995 |
| NTNG2 | 0.007995 |
| LUC7L3 | 0.007992 |
| HIST1H4H | 0.007991 |
| SLC26A7 | 0.007990 |
| TPBGL | 0.007988 |
| KIAA1549 | 0.007986 |
| NNAT | 0.007980 |
| VWCE | 0.007974 |
| NKX6-2 | 0.007971 |
| CAPRIN2 | 0.007967 |
| FNDC5 | 0.007966 |
| ZNF43 | 0.007965 |
| MPP2 | 0.007964 |
| MTHFD2 | 0.007964 |
| DPH7 | 0.007963 |
| CBX8 | 0.007960 |
| NREP | 0.007959 |
| GJC3 | 0.007957 |
| NPB | 0.007956 |
| LUC7L | 0.007956 |
| VN1R1 | 0.007954 |
| PROM1 | 0.007944 |
| UHRF2 | 0.007941 |
| MAP2 | 0.007940 |
| RAP1GAP2 | 0.007939 |
| HOXC11 | 0.007938 |
| KIF22 | 0.007937 |
| NUP210 | 0.007934 |
| CNGA2 | 0.007934 |
| ACVR1C | 0.007924 |
| DNMT3A | 0.007923 |
| ACVR2B | 0.007921 |
| TRIM73 | 0.007916 |
| PCYT1B | 0.007915 |
| DMP1 | 0.007912 |
| ZNF829 | 0.007911 |
| GAL3ST2 | 0.007910 |
| HEPHL1 | 0.007909 |
| TPD52 | 0.007908 |
| DHFR | 0.007908 |
| MYO7A | 0.007902 |
| GOLGA8B | 0.007900 |
| TTC23L | 0.007900 |
| ARL9 | 0.007898 |
| KALRN | 0.007890 |
| CEP95 | 0.007883 |
| TLX2 | 0.007881 |
| ZNF737 | 0.007879 |
| MEGF11 | 0.007879 |
| LGR4 | 0.007878 |
| TRH | 0.007877 |
| PTCH2 | 0.007875 |
| PRDM12 | 0.007875 |
| PRIM1 | 0.007869 |
| ZFP37 | 0.007866 |
| BRINP1 | 0.007861 |
| ZDHHC23 | 0.007857 |
| PTF1A | 0.007855 |
| WSCD1 | 0.007848 |
| DBX1 | 0.007845 |
| RETNLB | 0.007841 |
| ZNF550 | 0.007838 |
| SAAL1 | 0.007832 |
| TTLL2 | 0.007832 |
| ZNF273 | 0.007830 |
| PRAC1 | 0.007827 |
| LTB4R | 0.007823 |
| CLEC2L | 0.007823 |
| GADD45G | 0.007808 |
| MCM6 | 0.007806 |
| ADAM21 | 0.007805 |
| TAS2R30 | 0.007798 |
| GALNT8 | 0.007792 |
| C12orf75 | 0.007792 |
| TFAP4 | 0.007784 |
| ZNF491 | 0.007783 |
| RFX3 | 0.007782 |
| MCF2 | 0.007780 |
| ZNF841 | 0.007774 |
| CDHR5 | 0.007772 |
| PNISR | 0.007770 |
| MC1R | 0.007768 |
| ZNF382 | 0.007763 |
| NT5DC3 | 0.007761 |
| ZNF620 | 0.007758 |
| SLC35F3 | 0.007755 |
| ZNF138 | 0.007755 |
| SLC25A21 | 0.007755 |
| KCNJ13 | 0.007751 |
| MEGF10 | 0.007749 |
| ANKRD20A3 | 0.007748 |
| OGT | 0.007745 |
| GPSM2 | 0.007744 |
| GCM1 | 0.007742 |
| DSN1 | 0.007737 |
| CCHCR1 | 0.007736 |
| MAGEB17 | 0.007733 |
| GLRA4 | 0.007721 |
| DMTF1 | 0.007717 |
| FAM71F1 | 0.007714 |
| CFHR1 | 0.007697 |
| TMPRSS15 | 0.007693 |
| OXT | 0.007690 |
| CNTLN | 0.007680 |
| PARP2 | 0.007676 |
| B3GNT4 | 0.007671 |
| SPDYE2 | 0.007670 |
| TAC1 | 0.007668 |
| ZNF8 | 0.007668 |
| SFXN1 | 0.007662 |
| MAGEL2 | 0.007661 |
| SLC23A3 | 0.007656 |
| C11orf94 | 0.007645 |
| TRIT1 | 0.007643 |
| DIAPH3 | 0.007642 |
| PCNT | 0.007640 |
| TECTA | 0.007638 |
| GTPBP3 | 0.007630 |
| FBXO45 | 0.007630 |
| ACOT12 | 0.007628 |
| GFAP | 0.007624 |
| NHS | 0.007623 |
| LENG8 | 0.007623 |
| THOP1 | 0.007612 |
| MICAL3 | 0.007611 |
| FAM19A4 | 0.007611 |
| GRIA4 | 0.007608 |
| TAS2R4 | 0.007608 |
| PLXNB3 | 0.007605 |
| MDM4 | 0.007603 |
| ZNF530 | 0.007600 |
| ZNF560 | 0.007598 |
| JRK | 0.007593 |
| NPIPB4 | 0.007593 |
| TDP1 | 0.007592 |
| ARVCF | 0.007590 |
| RAB6B | 0.007587 |
| USH1G | 0.007584 |
| TMEM132E | 0.007583 |
| VCX3A | 0.007574 |
| KCNV2 | 0.007570 |
| HTR1A | 0.007564 |
| LRP8 | 0.007563 |
| GALNT9 | 0.007557 |
| NAV1 | 0.007556 |
| NUP155 | 0.007553 |
| TGFBR3L | 0.007549 |
| VSTM5 | 0.007547 |
| ANKZF1 | 0.007547 |
| TBC1D3L | 0.007546 |
| GJC1 | 0.007541 |
| MS4A8 | 0.007541 |
| RGS20 | 0.007539 |
| TAS2R20 | 0.007536 |
| L2HGDH | 0.007535 |
| SLC16A1 | 0.007534 |
| ACTL8 | 0.007533 |
| CEP76 | 0.007530 |
| RBL1 | 0.007528 |
| PM20D2 | 0.007528 |
| OTUD7A | 0.007527 |
| DQX1 | 0.007523 |
| FHOD3 | 0.007522 |
| FAM156A | 0.007518 |
| MDM1 | 0.007517 |
| RHBG | 0.007515 |
| DOCK3 | 0.007513 |
| HAUS8 | 0.007505 |
| OTUD3 | 0.007505 |
| ZNF177 | 0.007503 |
| PLGLB2 | 0.007500 |
| REC8 | 0.007499 |
| KRTAP5-3 | 0.007497 |
| PRR4 | 0.007496 |
| ESRRB | 0.007489 |
| EPHA6 | 0.007486 |
| DMC1 | 0.007486 |
| SOAT2 | 0.007485 |
| METTL4 | 0.007483 |
| TAS2R38 | 0.007479 |
| SARM1 | 0.007469 |
| ZSCAN1 | 0.007468 |
| H2BFM | 0.007467 |
| KRTAP5-2 | 0.007465 |
| LCTL | 0.007462 |
| DLL1 | 0.007459 |
| SMTNL1 | 0.007457 |
| RIMBP3B | 0.007457 |
| ZWILCH | 0.007456 |
| GNG8 | 0.007454 |
| SLC7A3 | 0.007449 |
| DHRS13 | 0.007447 |
| CSAD | 0.007445 |
| NOP56 | 0.007435 |
| FAM193B | 0.007432 |
| SOGA1 | 0.007432 |
| ZNF572 | 0.007431 |
| FGFR1OP | 0.007429 |
| RRM1 | 0.007424 |
| SLX4IP | 0.007422 |
| TSSK3 | 0.007421 |
| FBXL21 | 0.007414 |
| UCP3 | 0.007413 |
| EBF2 | 0.007413 |
| ANKRD30BL | 0.007411 |
| CTU1 | 0.007410 |
| ZBTB20 | 0.007410 |
| DDTL | 0.007405 |
| CEP128 | 0.007403 |
| EFCAB13 | 0.007395 |
| BTN1A1 | 0.007393 |
| ZNF749 | 0.007392 |
| ANO8 | 0.007392 |
| WDR17 | 0.007390 |
| CLK2 | 0.007390 |
| PBX4 | 0.007390 |
| ARHGAP39 | 0.007389 |
| HIST1H2BB | 0.007388 |
| PURG | 0.007387 |
| KIAA1586 | 0.007386 |
| TCTEX1D2 | 0.007386 |
| USP1 | 0.007385 |
| CKAP5 | 0.007383 |
| METTL11B | 0.007379 |
| TMPRSS11A | 0.007376 |
| PGF | 0.007375 |
| PIAS2 | 0.007375 |
| KLK4 | 0.007373 |
| GNAT1 | 0.007369 |
| RTEL1 | 0.007368 |
| ZNF124 | 0.007367 |
| ZNF680 | 0.007362 |
| CENPT | 0.007361 |
| SLC25A29 | 0.007359 |
| NPBWR1 | 0.007357 |
| ZNF85 | 0.007355 |
| KLK14 | 0.007355 |
| PLA2G4D | 0.007352 |
| GPR179 | 0.007350 |
| ISL2 | 0.007344 |
| SLC30A8 | 0.007343 |
| KLHL17 | 0.007336 |
| ZNF286B | 0.007335 |
| MUTYH | 0.007334 |
| TMEM240 | 0.007332 |
| MSH6 | 0.007329 |
| LRRC37A | 0.007326 |
| ALG1L | 0.007321 |
| CYP26A1 | 0.007320 |
| STOX1 | 0.007318 |
| SMAD9 | 0.007317 |
| CYP46A1 | 0.007316 |
| NPY | 0.007315 |
| TMEM262 | 0.007312 |
| SYT11 | 0.007310 |
| PKD1L3 | 0.007309 |
| SFI1 | 0.007304 |
| EDRF1 | 0.007302 |
| TTC32 | 0.007302 |
| CDK5RAP3 | 0.007301 |
| PRL | 0.007296 |
| SYNGAP1 | 0.007295 |
| SETBP1 | 0.007294 |
| MIS18A | 0.007294 |
| SPEG | 0.007292 |
| C1QTNF3 | 0.007290 |
| CRP | 0.007286 |
| SYT9 | 0.007286 |
| CNBD2 | 0.007285 |
| OTOR | 0.007283 |
| ZNF473 | 0.007275 |
| FNDC9 | 0.007275 |
| PHF19 | 0.007275 |
| CLASRP | 0.007273 |
| ZDHHC13 | 0.007271 |
| C8orf86 | 0.007271 |
| NUDT11 | 0.007268 |
| ZNF292 | 0.007268 |
| BEST2 | 0.007266 |
| NARF | 0.007265 |
| WDR72 | 0.007264 |
| 43720 | 0.007259 |
| POLG2 | 0.007257 |
| CCDC73 | 0.007251 |
| SNRNP70 | 0.007250 |
| BEX2 | 0.007245 |
| OXCT1 | 0.007245 |
| GFI1 | 0.007240 |
| IL17A | 0.007236 |
| ZNF569 | 0.007232 |
| FANCM | 0.007228 |
| LIME1 | 0.007227 |
| HAUS6 | 0.007227 |
| SCN3B | 0.007226 |
| GSX1 | 0.007223 |
| OPCML | 0.007223 |
| DDIAS | 0.007221 |
| DTX3 | 0.007220 |
| PKD2L2 | 0.007218 |
| ABCG5 | 0.007218 |
| ZNF81 | 0.007217 |
| ANO9 | 0.007216 |
| TIA1 | 0.007215 |
| SLC26A6 | 0.007214 |
| GLRA2 | 0.007212 |
| SLC35G2 | 0.007211 |
| CDH4 | 0.007211 |
| GRPR | 0.007206 |
| RFPL2 | 0.007203 |
| PLCB1 | 0.007201 |
| RFX8 | 0.007200 |
| GREM1 | 0.007197 |
| NINL | 0.007196 |
| HOXB4 | 0.007194 |
| ANKRD26 | 0.007193 |
| PIRT | 0.007187 |
| CCDC62 | 0.007186 |
| ZNF675 | 0.007185 |
| SLC6A3 | 0.007185 |
| AFP | 0.007184 |
| EFHC1 | 0.007177 |
| ZNF91 | 0.007175 |
| XKR6 | 0.007175 |
| PLXNA3 | 0.007175 |
| HOXC5 | 0.007174 |
| SLC13A5 | 0.007168 |
| CDKL4 | 0.007167 |
| SLC3A1 | 0.007161 |
| SEC14L5 | 0.007160 |
| ZNF605 | 0.007159 |
| ZNF141 | 0.007157 |
| TDG | 0.007155 |
| FAM229A | 0.007154 |
| FAM131B | 0.007153 |
| NPIPA8 | 0.007153 |
| EPB41 | 0.007151 |
| TBC1D29 | 0.007148 |
| SLC35G6 | 0.007148 |
| SHPRH | 0.007146 |
| TMEM158 | 0.007146 |
| MAGEA4 | 0.007145 |
| MCIDAS | 0.007134 |
| C2orf66 | 0.007132 |
| MNS1 | 0.007130 |
| CTPS1 | 0.007130 |
| TUBB2A | 0.007130 |
| FANCC | 0.007127 |
| ZNF443 | 0.007123 |
| STK32A | 0.007122 |
| RBM4B | 0.007121 |
| NWD1 | 0.007121 |
| RPL3L | 0.007119 |
| ZNF607 | 0.007119 |
| TMTC4 | 0.007112 |
| ZNF417 | 0.007112 |
| EFCAB7 | 0.007109 |
| VCX | 0.007103 |
| MTRNR2L3 | 0.007103 |
| SPATS2 | 0.007103 |
| LY6H | 0.007102 |
| CBX1 | 0.007099 |
| RDH16 | 0.007097 |
| ZNF497 | 0.007084 |
| G2E3 | 0.007083 |
| BCL2 | 0.007080 |
| CERS6 | 0.007080 |
| SLC2A1 | 0.007077 |
| ZCCHC11 | 0.007075 |
| PRR3 | 0.007075 |
| SYCP3 | 0.007073 |
| MYH6 | 0.007067 |
| PLAC8L1 | 0.007065 |
| ILF3 | 0.007065 |
| CMTM1 | 0.007062 |
| UCN | 0.007058 |
| SLC7A1 | 0.007051 |
| BUB3 | 0.007050 |
| NPIPB6 | 0.007045 |
| TBX15 | 0.007044 |
| SV2C | 0.007040 |
| KHDC1L | 0.007040 |
| MTA1 | 0.007039 |
| CCNA1 | 0.007038 |
| CNNM1 | 0.007038 |
| SAC3D1 | 0.007032 |
| KLHL11 | 0.007032 |
| GABBR1 | 0.007031 |
| PTPRT | 0.007029 |
| CREB3L4 | 0.007024 |
| SLC25A34 | 0.007022 |
| LRR1 | 0.007022 |
| GAS2 | 0.007020 |
| LSMEM1 | 0.007017 |
| TRIM74 | 0.007016 |
| NXPH2 | 0.007015 |
| CIB2 | 0.007013 |
| CABLES2 | 0.007011 |
| LMAN1L | 0.007010 |
| GC | 0.007010 |
| TMEM132B | 0.007002 |
| GCH1 | 0.007000 |
| POC1A | 0.006998 |
| PIPOX | 0.006997 |
| BARHL2 | 0.006994 |
| CCDC175 | 0.006994 |
| MBTD1 | 0.006994 |
| GIGYF1 | 0.006994 |
| ZNF345 | 0.006991 |
| RASD2 | 0.006988 |
| EGLN3 | 0.006988 |
| CRB1 | 0.006988 |
| SOX12 | 0.006987 |
| HIST1H2BD | 0.006981 |
| ZAR1 | 0.006980 |
| UQCC2 | 0.006979 |
| CHORDCI | 0.006973 |
| SYCP2L | 0.006972 |
| PCGF3 | 0.006971 |
| LZTS1 | 0.006968 |
| CFAP44 | 0.006967 |
| FOXA1 | 0.006962 |
| DEK | 0.006959 |
| GDPD4 | 0.006958 |
| ARL10 | 0.006956 |
| LRRC37B | 0.006956 |
| C22orf42 | 0.006950 |
| C6orf141 | 0.006949 |
| ANKRD24 | 0.006949 |
| KLHL31 | 0.006947 |
| SGCZ | 0.006946 |
| ZNF112 | 0.006940 |
| GPR22 | 0.006937 |
| ASIC1 | 0.006935 |
| FSCN3 | 0.006932 |
| PABPC1L2B | 0.006931 |
| BEND3 | 0.006929 |
| CITED1 | 0.006928 |
| SLC26A5 | 0.006927 |
| ZNF266 | 0.006925 |
| HTR5A | 0.006924 |
| HILPDA | 0.006922 |
| TP53TG3C | 0.006920 |
| POPDC3 | 0.006919 |
| DEPDC4 | 0.006911 |
| TAS2R3 | 0.006904 |
| FAM184B | 0.006893 |
| RNPC3 | 0.006893 |
| RGS11 | 0.006892 |
| CNGA3 | 0.006891 |
| ZNF648 | 0.006889 |
| CBWD7 | 0.006888 |
| FAN1 | 0.006883 |
| NTSR2 | 0.006883 |
| CTCFL | 0.006881 |
| STK36 | 0.006877 |
| SHC2 | 0.006875 |
| CDK19 | 0.006871 |
| ZFP62 | 0.006870 |
| TSPYL4 | 0.006869 |
| HSF2 | 0.006869 |
| SLC38A1 | 0.006868 |
| CNTF | 0.006863 |
| DCAF4L1 | 0.006853 |
| KLF14 | 0.006852 |
| 43530 | 0.006843 |
| GPR152 | 0.006841 |
| FSTL4 | 0.006839 |
| DCAF16 | 0.006838 |
| ALS2CR11 | 0.006837 |
| PLA2G4B | 0.006836 |
| KLF12 | 0.006836 |
| CHD6 | 0.006835 |
| MST1 | 0.006835 |
| AGAP9 | 0.006831 |
| ERICH6B | 0.006831 |
| APOA2 | 0.006828 |
| ZNF460 | 0.006824 |
| KAT2A | 0.006822 |
| CCSAP | 0.006820 |
| MAFA | 0.006817 |
| UNG | 0.006817 |
| HIST1H2BI | 0.006813 |
| RAD52 | 0.006812 |
| TAS2R14 | 0.006812 |
| HDAC2 | 0.006811 |
| NLGN4X | 0.006806 |
| UBN2 | 0.006804 |
| CLCN2 | 0.006803 |
| KIF4B | 0.006802 |
| GLMN | 0.006799 |
| USP37 | 0.006798 |
| HMGN2 | 0.006793 |
| CHST5 | 0.006793 |
| HOXB6 | 0.006792 |
| FAM76B | 0.006791 |
| DCST2 | 0.006790 |
| SH3D21 | 0.006790 |
| FAM122B | 0.006787 |
| GALNT16 | 0.006785 |
| MCM3 | 0.006783 |
| ZNF254 | 0.006779 |
| ENC1 | 0.006774 |
| ZNF774 | 0.006773 |
| ZNF26 | 0.006773 |
| ZNF700 | 0.006772 |
| ECE2 | 0.006768 |
| TMEM59L | 0.006766 |
| SORD | 0.006764 |
| VRK1 | 0.006758 |
| PRKDC | 0.006757 |
| UBXN7 | 0.006756 |
| RALGPS1 | 0.006755 |
| GJD2 | 0.006752 |
| C7orf33 | 0.006751 |
| NOS1AP | 0.006750 |
| MTRNR2L8 | 0.006745 |
| SNRNP48 | 0.006738 |
| KIF21B | 0.006737 |
| ZNF814 | 0.006736 |
| WRAP73 | 0.006727 |
| FAM163A | 0.006722 |
| MYSM1 | 0.006718 |
| HSPA4L | 0.006716 |
| RGPD3 | 0.006715 |
| GPR37L1 | 0.006714 |
| FBLN7 | 0.006710 |
| DCUN1D2 | 0.006709 |
| BTBD3 | 0.006708 |
| CFHR4 | 0.006707 |
| HIST1H1T | 0.006707 |
| KCNK2 | 0.006705 |
| CPSF6 | 0.006704 |
| FGF9 | 0.006700 |
| ADAMTSL1 | 0.006698 |
| STRBP | 0.006686 |
| SLC15A1 | 0.006685 |
| ADM2 | 0.006680 |
| GRK7 | 0.006676 |
| RIT2 | 0.006672 |
| ZNF10 | 0.006671 |
| ZNF227 | 0.006669 |
| KIAA1841 | 0.006665 |
| ZBTB26 | 0.006663 |
| ZNF567 | 0.006663 |
| C10orf71 | 0.006662 |
| NDC1 | 0.006661 |
| HOXB5 | 0.006660 |
| MOK | 0.006657 |
| TMOD2 | 0.006656 |
| MTRNR2L4 | 0.006656 |
| ACTL6A | 0.006654 |
| SSTR5 | 0.006651 |
| AP1G2 | 0.006651 |
| QSER1 | 0.006645 |
| SATB2 | 0.006643 |
| C1orf159 | 0.006639 |
| CPA1 | 0.006637 |
| HIST2H2BF | 0.006637 |
| VAMP1 | 0.006636 |
| CACNB2 | 0.006635 |
| PRPF3 | 0.006635 |
| YEATS2 | 0.006634 |
| ZNF248 | 0.006633 |
| PPP1R27 | 0.006633 |
| JSRP1 | 0.006631 |
| PHF8 | 0.006629 |
| MCMDC2 | 0.006626 |
| PAICS | 0.006622 |
| STARD6 | 0.006618 |
| IFT81 | 0.006617 |
| STC2 | 0.006616 |
| DLG5 | 0.006616 |
| DDX39B | 0.006615 |
| RTTN | 0.006614 |
| BTBD18 | 0.006607 |
| ADAMTS13 | 0.006606 |
| ZNF667 | 0.006606 |
| TAS2R13 | 0.006606 |
| SNX32 | 0.006605 |
| BAIAP2L2 | 0.006605 |
| EGFL8 | 0.006603 |
| LENEP | 0.006602 |
| LCORL | 0.006598 |
| DGKE | 0.006598 |
| RIC8B | 0.006595 |
| BORA | 0.006593 |
| ZNF883 | 0.006591 |
| ZCCHC12 | 0.006589 |
| STXBP5 | 0.006586 |
| ZCCHC16 | 0.006585 |
| ZNF251 | 0.006584 |
| CDH8 | 0.006583 |
| DDX55 | 0.006581 |
| ZNF184 | 0.006580 |
| NANOS3 | 0.006576 |
| ZNF285 | 0.006575 |
| SDR9C7 | 0.006575 |
| DNAJC5G | 0.006573 |
| LRRC49 | 0.006571 |
| PNPLA5 | 0.006570 |
| C11orf80 | 0.006569 |
| RBM15 | 0.006568 |
| NDUFAF5 | 0.006567 |
| IMPG2 | 0.006566 |
| TTC3 | 0.006565 |
| WNK4 | 0.006565 |
| ZSCAN9 | 0.006563 |
| INPPL1 | 0.006562 |
| ZNF280C | 0.006559 |
| SLC6A19 | 0.006558 |
| VCX3B | 0.006556 |
| TNFRSF11A | 0.006555 |
| N4BP2 | 0.006554 |
| ADGRF3 | 0.006552 |
| PPP2R2B | 0.006551 |
| PAXIP1 | 0.006550 |
| HNRNPH1 | 0.006549 |
| S100PBP | 0.006549 |
| ZNF397 | 0.006549 |
| TMEM191C | 0.006544 |
| MIP | 0.006544 |
| UNC13C | 0.006543 |
| NR1I2 | 0.006542 |
| ZSCAN16 | 0.006540 |
| TCERG1 | 0.006536 |
| NPIPB11 | 0.006535 |
| C12orf50 | 0.006535 |
| ICA1 | 0.006533 |
| NEURL1B | 0.006533 |
| MDK | 0.006532 |
| TFDP1 | 0.006529 |
| PLA2G6 | 0.006529 |
| MAGEA8 | 0.006528 |
| C17orf78 | 0.006528 |
| ADAM12 | 0.006527 |
| CLIP3 | 0.006526 |
| SPHKAP | 0.006525 |
| ZSCAN12 | 0.006523 |
| C6orf163 | 0.006523 |
| SRSF11 | 0.006520 |
| NMNAT2 | 0.006512 |
| KRT20 | 0.006512 |
| MRGPRE | 0.006511 |
| LRIT2 | 0.006508 |
| ACTR3B | 0.006508 |
| PSPH | 0.006506 |
| RUFY4 | 0.006501 |
| CYP2W1 | 0.006501 |
| ZNF705D | 0.006499 |
| CPNE6 | 0.006496 |
| ANKRD7 | 0.006496 |
| ZNF439 | 0.006495 |
| CBX5 | 0.006493 |
| ZMYND19 | 0.006491 |
| CHML | 0.006490 |
| HAPLN2 | 0.006490 |
| P0U5F1B | 0.006489 |
| LPGAT1 | 0.006489 |
| FAM69C | 0.006489 |
| C10orf82 | 0.006488 |
| POLR3G | 0.006484 |
| CKS1B | 0.006483 |
| MSS51 | 0.006482 |
| PDZD7 | 0.006482 |
| ZNF354B | 0.006480 |
| ALKBH6 | 0.006480 |
| ZNF195 | 0.006478 |
| ELOVL7 | 0.006478 |
| HCRT | 0.006475 |
| CEBPG | 0.006474 |
| CHEK2 | 0.006473 |
| NTF3 | 0.006469 |
| TRPC1 | 0.006463 |
| CCDC77 | 0.006463 |
| A3GALT2 | 0.006461 |
| ZNF229 | 0.006451 |
| FGFBP3 | 0.006449 |
| U2SURP | 0.006447 |
| AKAP8L | 0.006446 |
| TAS2R46 | 0.006444 |
| KCTD16 | 0.006441 |
| ZNF235 | 0.006441 |
| HOXC13 | 0.006441 |
| PPEF2 | 0.006439 |
| PRSS37 | 0.006435 |
| LRRN2 | 0.006434 |
| ZNF337 | 0.006433 |
| PROP1 | 0.006431 |
| TNK2 | 0.006430 |
| CLHC1 | 0.006426 |
| BHMT | 0.006426 |
| ZNF326 | 0.006425 |
| EML5 | 0.006424 |
| POMT2 | 0.006424 |
| POLDI | 0.006422 |
| C9orf131 | 0.006422 |
| FAM155A | 0.006422 |
| TMC3 | 0.006420 |
| HOXA6 | 0.006419 |
| IZUMO1 | 0.006417 |
| FBXO16 | 0.006416 |
| SRSF10 | 0.006415 |
| PTPDC1 | 0.006414 |
| MPP6 | 0.006411 |
| ZSCAN30 | 0.006404 |
| MAMSTR | 0.006397 |
| PDCL2 | 0.006394 |
| DEPDC7 | 0.006392 |
| GDPD2 | 0.006391 |
| ZNF514 | 0.006391 |
| TBX20 | 0.006391 |
| ADGRF2 | 0.006390 |
| RIF1 | 0.006387 |
| ATG4D | 0.006386 |
| DUSP28 | 0.006385 |
| TMEM150C | 0.006381 |
| NPIPB9 | 0.006378 |
| DOT1L | 0.006377 |
| HACE1 | 0.006375 |
| PRR22 | 0.006373 |
| GBX1 | 0.006367 |
| TSPYL6 | 0.006364 |
| CIT | 0.006364 |
| DUSP15 | 0.006362 |
| ZNF682 | 0.006361 |
| TMEM86B | 0.006350 |
| H2AFZ | 0.006350 |
| C12orf73 | 0.006347 |
| HECTD4 | 0.006345 |
| DHRS2 | 0.006345 |
| EFR3B | 0.006343 |
| XPO5 | 0.006343 |
| ZKSCAN1 | 0.006343 |
| TRIM37 | 0.006340 |
| BICD1 | 0.006337 |
| PXYLP1 | 0.006337 |
| CLK1 | 0.006337 |
| ZNF568 | 0.006336 |
| LRRC37A2 | 0.006333 |
| ZNF419 | 0.006332 |
| ZNF100 | 0.006331 |
| TK1 | 0.006330 |
| AKAP17A | 0.006330 |
| LGI2 | 0.006328 |
| LYSMD1 | 0.006328 |
| ZNF566 | 0.006328 |
| ZNF709 | 0.006327 |
| GDI1 | 0.006326 |
| PDE7A | 0.006326 |
| CYB5RL | 0.006322 |
| BEND5 | 0.006321 |
| INHBC | 0.006314 |
| SIAH3 | 0.006314 |
| CEP135 | 0.006311 |
| PKLR | 0.006309 |
| ZNF135 | 0.006308 |
| BEND4 | 0.006299 |
| PTS | 0.006297 |
| MZF1 | 0.006297 |
| MYRFL | 0.006296 |
| SMCHD1 | 0.006295 |
| TMEM206 | 0.006291 |
| RAG2 | 0.006290 |
| SERPINA11 | 0.006287 |
| ITIH6 | 0.006286 |
| KDM1A | 0.006286 |
| MAP3K10 | 0.006285 |
| UBAP1L | 0.006285 |
| TMEM191B | 0.006285 |
| C1GALT1C1L | 0.006283 |
| RAB3IP | 0.006283 |
| LMX1A | 0.006281 |
| MIS18BP1 | 0.006280 |
| KRTAP17-1 | 0.006280 |
| PPIP5K1 | 0.006280 |
| CLNK | 0.006277 |
| HIST1H4L | 0.006272 |
| PKDREJ | 0.006269 |
| SLC32A1 | 0.006268 |
| PPP3R2 | 0.006268 |
| HOXC12 | 0.006267 |
| CDK2 | 0.006266 |
| ACAD11 | 0.006265 |
| SLC1A4 | 0.006264 |
| KEL | 0.006262 |
| NKTR | 0.006261 |
| POLA2 | 0.006259 |
| GEMIN2 | 0.006258 |
| ZNF280A | 0.006258 |
| HUNK | 0.006256 |
| SLC25A53 | 0.006255 |
| PBOV1 | 0.006253 |
| RPS6KA5 | 0.006248 |
| C5orf46 | 0.006247 |
| GLP1R | 0.006245 |
| ZRANB3 | 0.006243 |
| E2F5 | 0.006238 |
| POLN | 0.006235 |
| SPATA21 | 0.006235 |
| ZNF527 | 0.006232 |
| ARHGEF7 | 0.006232 |
| INTS7 | 0.006230 |
| PTOV1 | 0.006230 |
| CLPSL2 | 0.006228 |
| ALMS1 | 0.006227 |
| APCDD1L | 0.006226 |
| DMAP1 | 0.006225 |
| PABPC1L2A | 0.006223 |
| GABBR2 | 0.006223 |
| FKBP3 | 0.006219 |
| KCNIP3 | 0.006219 |
| NTSR1 | 0.006218 |
| FAM19A3 | 0.006214 |
| CTRL | 0.006212 |
| AGO2 | 0.006212 |
| C11orf84 | 0.006212 |
| RHO | 0.006211 |
| GPR148 | 0.006211 |
| TCEAL6 | 0.006211 |
| DCAF4L2 | 0.006211 |
| PRTFDC1 | 0.006209 |
| OXTR | 0.006209 |
| METTL3 | 0.006207 |
| LRFN4 | 0.006206 |
| TNNT1 | 0.006201 |
| FAM19A1 | 0.006200 |
| OVOL2 | 0.006200 |
| IGFN1 | 0.006200 |
| GPR52 | 0.006199 |
| KCNJ14 | 0.006198 |
| ZNF74 | 0.006197 |
| BRWD3 | 0.006197 |
| NUP85 | 0.006195 |
| MYO15B | 0.006193 |
| TNPO2 | 0.006193 |
| PRSS1 | 0.006191 |
| SSPO | 0.006190 |
| CCNO | 0.006187 |
| ZNF679 | 0.006184 |
| AP3M2 | 0.006182 |
| PLCG1 | 0.006180 |
| ZFP14 | 0.006180 |
| SLC25A14 | 0.006180 |
| SCGB2A1 | 0.006178 |
| DFFB | 0.006178 |
| ZNF718 | 0.006176 |
| SH2D7 | 0.006175 |
| ZNF239 | 0.006175 |
| SPATA33 | 0.006170 |
| SPDYE1 | 0.006169 |
| PFKP | 0.006168 |
| SPICE1 | 0.006167 |
| TCP10L2 | 0.006166 |
| PHIP | 0.006165 |
| CTAG1B | 0.006164 |
| NAIP | 0.006161 |
| CCDC40 | 0.006159 |
| TMEM132D | 0.006158 |
| TBX10 | 0.006157 |
| ABCG4 | 0.006152 |
| ULK3 | 0.006149 |
| FAM71F2 | 0.006148 |
| CSMD3 | 0.006146 |
| RCC2 | 0.006146 |
| PTH2 | 0.006146 |
| FOXRED2 | 0.006145 |
| TMEM81 | 0.006143 |
| SLC4A11 | 0.006142 |
| E4F1 | 0.006142 |
| RPAIN | 0.006141 |
| TBC1D3I | 0.006140 |
| SPATAI6 | 0.006139 |
| ZBTB25 | 0.006135 |
| ABCC11 | 0.006133 |
| ZNF3 | 0.006130 |
| GLIPR1L1 | 0.006128 |
| PROSER3 | 0.006125 |
| NDUFAF6 | 0.006123 |
| LRRC37A3 | 0.006121 |
| BNIP3 | 0.006120 |
| TSHZ2 | 0.006120 |
| SHMT2 | 0.006112 |
| NIPSNAP3B | 0.006112 |
| GALR3 | 0.006111 |
| KDM4E | 0.006111 |
| SRPK1 | 0.006110 |
| TIAM1 | 0.006108 |
| STK39 | 0.006105 |
| SP4 | 0.006105 |
| TUSC3 | 0.006105 |
| ZNF660 | 0.006104 |
| ASIC2 | 0.006101 |
| AIPL1 | 0.006100 |
| DHPS | 0.006100 |
| CDK8 | 0.006096 |
| ZNF529 | 0.006095 |
| POU4F3 | 0.006095 |
| TOP3B | 0.006095 |
| FANCF | 0.006092 |
| RAD51C | 0.006091 |
| SPPL2C | 0.006087 |
| XYLB | 0.006085 |
| CLDN20 | 0.006085 |
| ARNTL2 | 0.006084 |
| GDF11 | 0.006084 |
| SLC39A6 | 0.006081 |
| INTS8 | 0.006077 |
| DTYMK | 0.006076 |
| RANBP1 | 0.006075 |
| ATP6V0E2 | 0.006070 |
| GADL1 | 0.006069 |
| ZNF420 | 0.006069 |
| MSTN | 0.006065 |
| NBPF8 | 0.006064 |
| CPSF1 | 0.006064 |
| SLITRK3 | 0.006064 |
| JADE3 | 0.006064 |
| AQP12A | 0.006062 |
| PMS1 | 0.006061 |
| C17orf47 | 0.006057 |
| C1orf74 | 0.006052 |
| ZNF852 | 0.006051 |
| GPR151 | 0.006050 |
| ZNF551 | 0.006047 |
| SFPQ | 0.006047 |
| ZNF577 | 0.006043 |
| LRRC69 | 0.006043 |
| SOCS7 | 0.006042 |
| THAP9 | 0.006042 |
| PRKAA2 | 0.006039 |
| CT55 | 0.006037 |
| TUBE1 | 0.006036 |
| LRRC45 | 0.006036 |
| BRD8 | 0.006035 |
| ASRGL1 | 0.006034 |
| ZNF260 | 0.006033 |
| FBXL6 | 0.006032 |
| FAM71D | 0.006030 |
| ANGPT2 | 0.006029 |
| HOOK2 | 0.006027 |
| AADAT | 0.006026 |
| SYCE1L | 0.006025 |
| EED | 0.006023 |
| DLGAP1 | 0.006021 |
| BDKRB1 | 0.006020 |
| ZNF250 | 0.006020 |
| KCNJ10 | 0.006018 |
| GPR50 | 0.006017 |
| NCBP1 | 0.006015 |
| GLRA1 | 0.006015 |
| ATL1 | 0.006014 |
| BEST4 | 0.006012 |
| MZT1 | 0.006011 |
| ITGB1BP2 | 0.006010 |
| MTMR4 | 0.006006 |
| DLK2 | 0.006004 |
| TM6SF2 | 0.006001 |
| ASIC4 | 0.005996 |
| AMACR | 0.005995 |
| TIGD4 | 0.005994 |
| SLC9A7 | 0.005988 |
| LRRC63 | 0.005987 |
| ZNF461 | 0.005985 |
| HPRT1 | 0.005984 |
| HIST2H2BE | 0.005984 |
| MDC1 | 0.005983 |
| FOXD4L3 | 0.005982 |
| HYPK | 0.005982 |
| SRGAP1 | 0.005979 |
| CCDC142 | 0.005978 |
| CCDC130 | 0.005977 |
| ADCK5 | 0.005977 |
| PAPOLB | 0.005976 |
| ZNF302 | 0.005975 |
| ZNF543 | 0.005973 |
| ZNF232 | 0.005973 |
| SRRT | 0.005970 |
| BRWD1 | 0.005969 |
| NEUROD6 | 0.005968 |
| KIAA0895 | 0.005968 |
| RIMS4 | 0.005966 |
| ARGLU1 | 0.005964 |
| PXT1 | 0.005962 |
| ASAH2 | 0.005958 |
| TMEM201 | 0.005957 |
| C14orf80 | 0.005954 |
| FOXD4L4 | 0.005954 |
| DKC1 | 0.005954 |
| NPIPB8 | 0.005946 |
| PNMAL1 | 0.005942 |
| ZNF571 | 0.005940 |
| SLC9C1 | 0.005938 |
| ZNF772 | 0.005936 |
| SCLT1 | 0.005935 |
| TIGD7 | 0.005935 |
| CCDC178 | 0.005932 |
| HSD17B3 | 0.005930 |
| CEP89 | 0.005929 |
| ZNF283 | 0.005928 |
| HAGHL | 0.005928 |
| SORCS3 | 0.005924 |
| USP45 | 0.005923 |
| LRP5L | 0.005922 |
| XPO1 | 0.005920 |
| HECTD2 | 0.005917 |
| ZNF322 | 0.005916 |
| PCMTD2 | 0.005915 |
| NT5DC4 | 0.005914 |
| MAATS1 | 0.005911 |
| SLC16A14 | 0.005909 |
| AK9 | 0.005907 |
| C2CD4A | 0.005905 |
| PPP1R14B | 0.005902 |
| ATG16L2 | 0.005900 |
| WIPF3 | 0.005896 |
| USP17L7 | 0.005895 |
| DMRT1 | 0.005895 |
| IGSF21 | 0.005892 |
| VCPKMT | 0.005892 |
| KRTAP5-5 | 0.005890 |
| NPHP4 | 0.005890 |
| DMPK | 0.005889 |
| PLSCR2 | 0.005889 |
| DMRT3 | 0.005889 |
| RHD | 0.005888 |
| KCTD4 | 0.005887 |
| TTF2 | 0.005886 |
| C6orf222 | 0.005885 |
| TUBA1A | 0.005882 |
| ZNF432 | 0.005880 |
| HMX1 | 0.005878 |
| C15orf41 | 0.005877 |
| EFNB3 | 0.005876 |
| DXO | 0.005875 |
| CACNB3 | 0.005875 |
| METAP1D | 0.005875 |
| FAM60A | 0.005873 |
| CAPN10 | 0.005872 |
| HDAC6 | 0.005871 |
| TIPIN | 0.005871 |
| MYL6B | 0.005869 |
| C5orf42 | 0.005868 |
| ELL3 | 0.005868 |
| ZMYM1 | 0.005867 |
| BHLHB9 | 0.005866 |
| ZBTB37 | 0.005865 |
| SMS | 0.005864 |
| ING5 | 0.005859 |
| BASP1 | 0.005855 |
| PAFAH1B3 | 0.005854 |
| TMEM106C | 0.005851 |
| MAPK8 | 0.005849 |
| MTF2 | 0.005845 |
| H1FX | 0.005844 |
| GMPS | 0.005842 |
| ZKSCAN8 | 0.005840 |
| HAUS1 | 0.005838 |
| TCAP | 0.005838 |
| TMEM178A | 0.005838 |
| ASCL5 | 0.005836 |
| TPO | 0.005836 |
| MIB2 | 0.005835 |
| VANGL2 | 0.005834 |
| IL17C | 0.005832 |
| LIN9 | 0.005829 |
| ZNF418 | 0.005828 |
| SYN3 | 0.005827 |
| RBM38 | 0.005825 |
| FAM218A | 0.005824 |
| PIGX | 0.005820 |
| FAM186A | 0.005819 |
| GFRA4 | 0.005819 |
| ZNF669 | 0.005817 |
| CCDC57 | 0.005817 |
| FNDC7 | 0.005816 |
| CA7 | 0.005815 |
| BTAF1 | 0.005810 |
| AG03 | 0.005809 |
| RUFY3 | 0.005809 |
| PRR19 | 0.005808 |
| IL19 | 0.005808 |
| C5orf30 | 0.005807 |
| TAF2 | 0.005806 |
| PARD6G | 0.005802 |
| VPS13A | 0.005801 |
| CBWD3 | 0.005800 |
| SCRIB | 0.005800 |
| RTN2 | 0.005798 |
| CADM4 | 0.005797 |
| MICAL1 | 0.005797 |
| TAF1D | 0.005796 |
| CYP2C8 | 0.005794 |
| PSMG4 | 0.005791 |
| LRGUK | 0.005781 |
| LBX2 | 0.005781 |
| TSGA10 | 0.005776 |
| SAMD1 | 0.005775 |
| PHC1 | 0.005774 |
| ARHGEF33 | 0.005774 |
| FAM65C | 0.005774 |
| GPR75 | 0.005773 |
| DHX34 | 0.005773 |
| KCNE2 | 0.005768 |
| SNAPC3 | 0.005763 |
| NPIPB5 | 0.005760 |
| ZKSCAN7 | 0.005760 |
| ADGRL1 | 0.005760 |
| NEU3 | 0.005759 |
| SRMS | 0.005759 |
| C2CD4D | 0.005758 |
| SLC25A47 | 0.005757 |
| TMEM89 | 0.005756 |
| MEX3D | 0.005755 |
| TMEM31 | 0.005754 |
| LFNG | 0.005754 |
| LRRC14 | 0.005752 |
| ZMYM2 | 0.005749 |
| CKB | 0.005747 |
| TPTE | 0.005747 |
| R3HDM1 | 0.005746 |
| EXOSC2 | 0.005746 |
| ESR2 | 0.005745 |
| ZDBF2 | 0.005744 |
| CEP83 | 0.005744 |
| AKT3 | 0.005744 |
| SLC13A4 | 0.005743 |
| ZNF775 | 0.005743 |
| SLC6A11 | 0.005743 |
| AACS | 0.005742 |
| TMEM136 | 0.005739 |
| ZNF510 | 0.005738 |
| MRI1 | 0.005737 |
| SUZ12 | 0.005736 |
| HIST2H2AA3 | 0.005735 |
| ZNF404 | 0.005735 |
| CDC25B | 0.005730 |
| SLC25A2 | 0.005730 |
| EBLN2 | 0.005728 |
| DBNDD1 | 0.005725 |
| SENP5 | 0.005723 |
| MASTL | 0.005720 |
| ZNF611 | 0.005719 |
| KRTAP5-7 | 0.005718 |
| HDGFL1 | 0.005717 |
| TULP4 | 0.005714 |
| NUP43 | 0.005710 |
| CHRNA1 | 0.005709 |
| GRIN2D | 0.005708 |
| NAA11 | 0.005708 |
| TMEM181 | 0.005705 |
| ZNF704 | 0.005705 |
| ZNF785 | 0.005703 |
| FBXW7 | 0.005702 |
| ROBO1 | 0.005701 |
| ZNF776 | 0.005699 |
| POU2F1 | 0.005698 |
| ZFR2 | 0.005696 |
| RBMX | 0.005695 |
| CBWD5 | 0.005694 |
| ERICH6 | 0.005690 |
| TAF5 | 0.005690 |
| TACR3 | 0.005689 |
| LSM14B | 0.005689 |
| SACS | 0.005689 |
| BAALC | 0.005688 |
| ZNF333 | 0.005687 |
| EPχ | 0.005687 |
| TMEM260 | 0.005684 |
| DCAF8L1 | 0.005683 |
| MOGAT2 | 0.005683 |
| XPOT | 0.005682 |
| NSMF | 0.005682 |
| ZNF518B | 0.005678 |
| CPNE4 | 0.005677 |
| ZNF75A | 0.005675 |
| IVNS1ABP | 0.005675 |
| ZSWIM2 | 0.005673 |
| RBM44 | 0.005673 |
| SRSF2 | 0.005673 |
| ZNF549 | 0.005673 |
| RAD9A | 0.005664 |
| SLC2A2 | 0.005664 |
| ABCB6 | 0.005661 |
| ANK2 | 0.005657 |
| ZNF493 | 0.005654 |
| CRHBP | 0.005654 |
| WDR34 | 0.005652 |
| NCAPD2 | 0.005646 |
| POLR2H | 0.005645 |
| OPA1 | 0.005644 |
| SAMD15 | 0.005642 |
| TMED8 | 0.005641 |
| TRA2B | 0.005638 |
| PABPN1 | 0.005635 |
| TRAF5 | 0.005635 |
| AANAT | 0.005635 |
| TCF3 | 0.005633 |
| MTERF2 | 0.005631 |
| IL12RB2 | 0.005630 |
| RFX6 | 0.005626 |
| TADA2A | 0.005623 |
| NKX1-2 | 0.005622 |
| KCNMA1 | 0.005618 |
| ABAT | 0.005618 |
| ENOX1 | 0.005617 |
| NUP205 | 0.005617 |
| QTRT1 | 0.005614 |
| CAMK4 | 0.005613 |
| SLC28A1 | 0.005613 |
| NAA16 | 0.005613 |
| MYO15A | 0.005610 |
| CHAT | 0.005607 |
| PYCR1 | 0.005607 |
| C16orf87 | 0.005605 |
| GINS3 | 0.005604 |
| ZNF544 | 0.005601 |
| ODF2 | 0.005601 |
| RNF113B | 0.005599 |
| CCDC30 | 0.005597 |
| PRDM9 | 0.005594 |
| BCOR | 0.005594 |
| C2orf78 | 0.005591 |
| CSTF3 | 0.005590 |
| PRSS46 | 0.005589 |
| OR2H2 | 0.005588 |
| SLC6A8 | 0.005588 |
| UVSSA | 0.005586 |
| MARCKS | 0.005586 |
| ZNF180 | 0.005585 |
| MSANTD2 | 0.005585 |
| PHOSPHO2 | 0.005584 |
| CCNL1 | 0.005581 |
| RASGEF1C | 0.005579 |
| APOO | 0.005577 |
| DDX4 | 0.005575 |
| CCNJ | 0.005573 |
| PDIK1L | 0.005573 |
| PPP1R37 | 0.005572 |
| NUTM2G | 0.005572 |
| RPAP2 | 0.005568 |
| PDE6C | 0.005568 |
| ZNF528 | 0.005567 |
| ZNF224 | 0.005565 |
| CACNA1I | 0.005565 |
| NOX5 | 0.005563 |
| THSD7B | 0.005563 |
| HES7 | 0.005562 |
| ABHD1 | 0.005560 |
| BCL2L11 | 0.005559 |
| MAZ | 0.005558 |
| LARP1B | 0.005555 |
| TBPL1 | 0.005554 |
| GPR62 | 0.005553 |
| PHF10 | 0.005551 |
| UBR7 | 0.005550 |
| GGN | 0.005549 |
| ZNF133 | 0.005549 |
| TSSK6 | 0.005546 |
| CNDP1 | 0.005545 |
| NAT8 | 0.005544 |
| CFC1 | 0.005542 |
| BHLHE23 | 0.005541 |
| LRP3 | 0.005537 |
| DBN1 | 0.005536 |
| FAM129C | 0.005536 |
| CTAG1A | 0.005534 |
| SLC1A6 | 0.005534 |
| SLITRK5 | 0.005532 |
| EFCAB5 | 0.005531 |
| NXPE2 | 0.005529 |
| ADAMTS6 | 0.005528 |
| DOPEY1 | 0.005524 |
| HSPB11 | 0.005523 |
| LSM7 | 0.005522 |
| PARP1 | 0.005521 |
| ZBTB39 | 0.005520 |
| COQ10A | 0.005517 |
| PSD3 | 0.005515 |
| SCGB2B2 | 0.005515 |
| C8orf88 | 0.005515 |
| PIWIL2 | 0.005514 |
| TSSK4 | 0.005514 |
| ALPK2 | 0.005514 |
| KIF21A | 0.005513 |
| TBC1D16 | 0.005512 |
| STX1B | 0.005510 |
| LCOR | 0.005508 |
| HSF2BP | 0.005506 |
| RNF144A | 0.005504 |
| APOBEC3B | 0.005504 |
| USP6 | 0.005504 |
| SENP1 | 0.005503 |
| PELI2 | 0.005503 |
| KIF27 | 0.005503 |
| CCDC171 | 0.005503 |
| KRTAP4-1 | 0.005501 |
| ARHGAP32 | 0.005499 |
| C17orf75 | 0.005497 |
| PATE1 | 0.005494 |
| FUBP1 | 0.005492 |
| DIP2A | 0.005492 |
| CLDN3 | 0.005489 |
| USP17L2 | 0.005489 |
| ARL6IP1 | 0.005487 |
| NRXN2 | 0.005485 |
| DAZ4 | 0.005485 |
| PCED1A | 0.005484 |
| XKR9 | 0.005479 |
| ZNF559 | 0.005479 |
| CEP170 | 0.005478 |
| TRIMS2 | 0.005478 |
| EXO5 | 0.005477 |
| OTX1 | 0.005475 |
| CNGB3 | 0.005474 |
| KIF2A | 0.005474 |
| CCDC87 | 0.005474 |
| NTN5 | 0.005474 |
| CCDC144NL | 0.005474 |
| POLI | 0.005473 |
| TRDN | 0.005471 |
| ANKRD49 | 0.005471 |
| TAF1L | 0.005470 |
| MTHFD2L | 0.005470 |
| ANKRD30A | 0.005470 |
| SAXO1 | 0.005469 |
| ATP13A3 | 0.005469 |
| COL9A2 | 0.005466 |
| DGCR8 | 0.005466 |
| RPGRIP1L | 0.005465 |
| PPP1R35 | 0.005462 |
| RALGAPA1 | 0.005462 |
| RNF138 | 0.005461 |
| SYNGR1 | 0.005458 |
| SLC22A16 | 0.005458 |
| TAF4 | 0.005456 |
| KRI1 | 0.005451 |
| METTL17 | 0.005450 |
| GSDMB | 0.005449 |
| TEKT5 | 0.005447 |
| PPM1H | 0.005447 |
| FSIP1 | 0.005447 |
| TTLL3 | 0.005446 |
| ACAN | 0.005445 |
| RBP1 | 0.005445 |
| AGAP5 | 0.005445 |
| TOP3A | 0.005444 |
| TF | 0.005440 |
| NPIPA3 | 0.005439 |
| RPRD1A | 0.005438 |
| ANKRD20A2 | 0.005438 |
| PADI3 | 0.005437 |
| ASB18 | 0.005436 |
| PLK5 | 0.005435 |
| ROPN1 | 0.005435 |
| ZNF445 | 0.005434 |
| HAUS3 | 0.005434 |
| WEE1 | 0.005431 |
| ZNF385C | 0.005431 |
| FAM117B | 0.005430 |
| RGAG4 | 0.005429 |
| PAIP2B | 0.005428 |
| SLX4 | 0.005424 |
| KPTN | 0.005424 |
| NDST3 | 0.005422 |
| RLN2 | 0.005420 |
| RBBP7 | 0.005419 |
| TAS2R50 | 0.005417 |
| DOPEY2 | 0.005417 |
| UNKL | 0.005417 |
| LSM8 | 0.005414 |
| ANKRD10 | 0.005412 |
| CCDC83 | 0.005411 |
| POTEC | 0.005409 |
| UPP2 | 0.005408 |
| PSTK | 0.005407 |
| UMOD | 0.005407 |
| RGPD5 | 0.005407 |
| RPEL1 | 0.005407 |
| C2CD3 | 0.005405 |
| ATOH1 | 0.005403 |
| PHTF2 | 0.005401 |
| C19orf18 | 0.005401 |
| HKR1 | 0.005399 |
| OSBP2 | 0.005398 |
| FAM19A2 | 0.005396 |
| KRBA2 | 0.005394 |
| NONO | 0.005393 |
| C8orf82 | 0.005391 |
| ZFC3H1 | 0.005390 |
| ZNF771 | 0.005390 |
| ZNF518A | 0.005390 |
| GALR2 | 0.005389 |
| FAM118A | 0.005387 |
| MNAT1 | 0.005386 |
| TMEM44 | 0.005386 |
| EPO | 0.005386 |
| SETD6 | 0.005386 |
| ZIK1 | 0.005385 |
| PRODH2 | 0.005384 |
| ZNF506 | 0.005384 |
| CHRNG | 0.005383 |
| POP1 | 0.005382 |
| PAGE2B | 0.005379 |
| ZNF783 | 0.005376 |
| DZANK1 | 0.005376 |
| ZNF761 | 0.005376 |
| KIF26B | 0.005375 |
| NPDC1 | 0.005374 |
| ANKRD30B | 0.005374 |
| ATF5 | 0.005373 |
| ZNF507 | 0.005370 |
| ATP6V1B1 | 0.005369 |
| NPAP1 | 0.005369 |
| ATAD2B | 0.005369 |
| SGK2 | 0.005369 |
| TRMT13 | 0.005368 |
| FOXD4L5 | 0.005367 |
| SDR39U1 | 0.005365 |
| NEURL4 | 0.005364 |
| ZNF674 | 0.005363 |
| MZT2A | 0.005361 |
| RLN1 | 0.005360 |
| MIPOL1 | 0.005358 |
| HSPB9 | 0.005358 |
| ZRANB2 | 0.005357 |
| C17orf80 | 0.005356 |
| ZNF610 | 0.005354 |
| EIF4EBP1 | 0.005353 |
| PRSS27 | 0.005351 |
| ABCG8 | 0.005350 |
| ZNF532 | 0.005350 |
| CHRNA10 | 0.005349 |
| ZNF77 | 0.005349 |
| SREK1 | 0.005347 |
| RFWD3 | 0.005347 |
| RSPH6A | 0.005345 |
| PHKA2 | 0.005345 |
| FOXRED1 | 0.005343 |
| FNBP4 | 0.005343 |
| FAM181B | 0.005342 |
| SOBP | 0.005340 |
| SMC3 | 0.005340 |
| CRB2 | 0.005340 |
| MCHR1 | 0.005338 |
| TUBG1 | 0.005337 |
| PNMA1 | 0.005336 |
| LIMD2 | 0.005335 |
| C2CD5 | 0.005334 |
| SRGAP3 | 0.005334 |
| TUBGCP6 | 0.005334 |
| KLHL32 | 0.005331 |
| GOLM1 | 0.005330 |
| TUBA8 | 0.005329 |
| TSPYL2 | 0.005329 |
| REV3L | 0.005329 |
| RAE1 | 0.005328 |
| PNPLA7 | 0.005326 |
| GLS2 | 0.005326 |
| NAA25 | 0.005326 |
| DYRK2 | 0.005326 |
| GPATCH2L | 0.005325 |
| LMX1B | 0.005324 |
| RTN1 | 0.005323 |
| BPHL | 0.005323 |
| GPR135 | 0.005321 |
| ZNF804B | 0.005321 |
| GPRASP1 | 0.005319 |
| URB2 | 0.005319 |
| NUP107 | 0.005318 |
| RYR3 | 0.005317 |
| SPP1 | 0.005317 |
| CAD | 0.005315 |
| NRARP | 0.005314 |
| ZNF431 | 0.005312 |
| NRM | 0.005311 |
| ZNF117 | 0.005309 |
| KDELC1 | 0.005306 |
| PLEKHA6 | 0.005306 |
| LPAR2 | 0.005305 |
| LKAAEAR1 | 0.005305 |
| RBAK | 0.005304 |
| PTAR1 | 0.005303 |
| ZNF182 | 0.005302 |
| EXOSC8 | 0.005301 |
| LIMS3 | 0.005299 |
| PXMP2 | 0.005297 |
| PKIA | 0.005295 |
| UCKL1 | 0.005295 |
| CLDN9 | 0.005294 |
| ZNF131 | 0.005292 |
| PDE6A | 0.005292 |
| ZFP82 | 0.005292 |
| NXPE1 | 0.005291 |
| KCNC4 | 0.005290 |
| DROSHA | 0.005287 |
| IKBKAP | 0.005282 |
| LRP12 | 0.005279 |
| PIGL | 0.005276 |
| SLC27A5 | 0.005275 |
| MRPS26 | 0.005274 |
| LMOD2 | 0.005271 |
| KLC2 | 0.005270 |
| FAM102B | 0.005270 |
| SDK1 | 0.005265 |
| STKLD1 | 0.005264 |
| C10orf62 | 0.005261 |
| APCS | 0.005260 |
| BRIX1 | 0.005259 |
| WDR4 | 0.005249 |
| RSRC1 | 0.005244 |
| RUFY2 | 0.005243 |
| PRIM2 | 0.005242 |
| CCR10 | 0.005240 |
| SNX31 | 0.005240 |
| ZNF146 | 0.005239 |
| MBLAC1 | 0.005238 |
| ZNF496 | 0.005232 |
| TEDDM1 | 0.005231 |
| NBPF11 | 0.005230 |
| ZNF226 | 0.005230 |
| RGL3 | 0.005230 |
| MAP10 | 0.005228 |
| VSX1 | 0.005227 |
| DCST1 | 0.005227 |
| HMGN1 | 0.005226 |
| FASTKD1 | 0.005225 |
| USP17L1 | 0.005224 |
| CR1L | 0.005222 |
| ZNF236 | 0.005222 |
| MORN1 | 0.005220 |
| SCML1 | 0.005218 |
| SKA2 | 0.005218 |
| ST20 | 0.005218 |
| ARL17B | 0.005217 |
| SLC22A14 | 0.005216 |
| SIMC1 | 0.005215 |
| DHRS11 | 0.005215 |
| SPDYE6 | 0.005213 |
| ADAMDEC1 | 0.005213 |
| ING1 | 0.005210 |
| UPF3A | 0.005209 |
| CERS3 | 0.005208 |
| RBM39 | 0.005207 |
| CAPN14 | 0.005205 |
| SDR42E1 | 0.005204 |
| RHOT2 | 0.005204 |
| TRMT11 | 0.005203 |
| C1orf35 | 0.005202 |
| CPSF4 | 0.005201 |
| NAB1 | 0.005200 |
| KRTAP12-1 | 0.005199 |
| ZNF696 | 0.005199 |
| LRRC74B | 0.005197 |
| ANKS3 | 0.005196 |
| FAM111A | 0.005195 |
| MMACHC | 0.005195 |
| IDH2 | 0.005193 |
| ACTA1 | 0.005193 |
| INSR | 0.005193 |
| UBA6 | 0.005191 |
| UNK | 0.005191 |
| LCE1E | 0.005191 |
| PNKP | 0.005190 |
| ASB14 | 0.005185 |
| RBM41 | 0.005185 |
| C4orf26 | 0.005184 |
| H2BFWT | 0.005181 |
| MYO1H | 0.005181 |
| SLC20A1 | 0.005180 |
| CDK10 | 0.005178 |
| LBR | 0.005177 |
| CYTH2 | 0.005177 |
| MAP6 | 0.005177 |
| SAMD10 | 0.005176 |
| CCSER1 | 0.005173 |
| MPP4 | 0.005172 |
| H2AFB3 | 0.005172 |
| MT1H | 0.005170 |
| SNAPC4 | 0.005169 |
| NCS1 | 0.005169 |
| SLC35E2B | 0.005168 |
| GPC6 | 0.005168 |
| HES5 | 0.005166 |
| RHCE | 0.005165 |
| FAM71E2 | 0.005164 |
| SUN1 | 0.005163 |
| ZNF480 | 0.005163 |
| SOCS4 | 0.005162 |
| DPYSL3 | 0.005162 |
| CCDC112 | 0.005161 |
| CHRNA4 | 0.005160 |
| JAM3 | 0.005157 |
| KIZ | 0.005157 |
| PRDM10 | 0.005156 |
| YY2 | 0.005155 |
| LIPI | 0.005151 |
| TTC13 | 0.005151 |
| SLC5A11 | 0.005151 |
| SUPT16H | 0.005151 |
| PDCD2L | 0.005149 |
| TBC1D7 | 0.005148 |
| NUTM2F | 0.005147 |
| SLC27A2 | 0.005145 |
| ALG6 | 0.005144 |
| TEX9 | 0.005143 |
| NR2E1 | 0.005142 |
| ASAH2B | 0.005137 |
| AGAP1 | 0.005136 |
| NSUN6 | 0.005135 |
| CLDN15 | 0.005133 |
| BAHCC1 | 0.005132 |
| ZSCAN2 | 0.005131 |
| TUBA3E | 0.005128 |
| SNRPD1 | 0.005127 |
| CNTNAP4 | 0.005124 |
| ISYNA1 | 0.005121 |
| RBBP4 | 0.005121 |
| ADCY5 | 0.005120 |
| ZNF547 | 0.005119 |
| TTC21B | 0.005118 |
| TEX12 | 0.005116 |
| ZNF765 | 0.005113 |
| HEXDC | 0.005112 |
| VMAC | 0.005111 |
| PRDX2 | 0.005107 |
| RBM25 | 0.005105 |
| PCP2 | 0.005104 |
| SMC1A | 0.005103 |
| DMXL2 | 0.005102 |
| ZBED5 | 0.005101 |
| GCDH | 0.005101 |
| OR2L13 | 0.005097 |
| HSFY2 | 0.005097 |
| FAR1 | 0.005097 |
| RFC2 | 0.005097 |
| CAMK1D | 0.005096 |
| GTPBP2 | 0.005095 |
| ZBTB10 | 0.005095 |
| TSEN54 | 0.005095 |
| OR14A16 | 0.005092 |
| SLC25A33 | 0.005091 |
| GPR180 | 0.005090 |
| IFT122 | 0.005088 |
| TET3 | 0.005087 |
| PRPF40B | 0.005087 |
| ADGRV1 | 0.005087 |
| KDM4B | 0.005087 |
| FZR1 | 0.005085 |
| MGME1 | 0.005082 |
| FOXB2 | 0.005082 |
| TSNARE1 | 0.005078 |
| ZBED9 | 0.005076 |
| INPP5E | 0.005074 |
| UGT2B28 | 0.005074 |
| GCM2 | 0.005072 |
| RBM6 | 0.005072 |
| IL1RAPL1 | 0.005070 |
| CENPQ | 0.005070 |
| SRSF1 | 0.005070 |
| MTSS1 | 0.005067 |
| CCL25 | 0.005067 |
| KLC1 | 0.005066 |
| HFE2 | 0.005066 |
| IRS4 | 0.005065 |
| CARTPT | 0.005065 |
| IL1RAPL2 | 0.005065 |
| PLEKHD1 | 0.005065 |
| PFKFB4 | 0.005064 |
| FBN2 | 0.005063 |
| KLHL25 | 0.005061 |
| ZNF557 | 0.005059 |
| CRISP3 | 0.005057 |
| POGZ | 0.005054 |
| B3GALNT2 | 0.005053 |
| NLN | 0.005052 |
| FAM173B | 0.005052 |
| TP53TG5 | 0.005051 |
| OR2T33 | 0.005049 |
| GK5 | 0.005048 |
| AKAP5 | 0.005047 |
| EHMT2 | 0.005047 |
| CELF1 | 0.005047 |
| TTR | 0.005042 |
| FAM136A | 0.005039 |
| PRAP1 | 0.005039 |
| DVL3 | 0.005038 |
| CYP4F2 | 0.005036 |
| PABPC4L | 0.005035 |
| AQP6 | 0.005032 |
| ATAD3A | 0.005032 |
| SERGEF | 0.005031 |
| ARMC12 | 0.005031 |
| ELOVL6 | 0.005031 |
| RGPD8 | 0.005029 |
| PUS7L | 0.005028 |
| GHRHR | 0.005027 |
| KCNIP4 | 0.005026 |
| YIPF7 | 0.005024 |
| MSL1 | 0.005024 |
| TBC1D8 | 0.005023 |
| HSPA14 | 0.005021 |
| C1orf109 | 0.005019 |
| TRIM3 | 0.005018 |
| ARID3C | 0.005016 |
| ACTL10 | 0.005014 |
| SOX30 | 0.005013 |
| TAFIA | 0.005013 |
| ZNF318 | 0.005012 |
| GTF2IRD1 | 0.005009 |
| TGIF2LX | 0.005004 |
| UBE2O | 0.005002 |
| GRIP2 | 0.005001 |
| ZNF623 | 0.005000 |
| MAP3K9 | 0.005000 |
| ARMCX4 | 0.004999 |
| DNM1L | 0.004997 |
| ZDHHC17 | 0.004996 |
| MAP3K12 | 0.004995 |
| BOP1 | 0.004993 |
| BDP1 | 0.004992 |
| PRDM7 | 0.004991 |
| ZNF215 | 0.004991 |
| IQCB1 | 0.004986 |
| VAV2 | 0.004985 |
| LIG3 | 0.004985 |
| MLANA | 0.004983 |
| ZNF546 | 0.004983 |
| MYRIP | 0.004982 |
| C10orf131 | 0.004980 |
| C10orf35 | 0.004977 |
| ZNF662 | 0.004977 |
| BRF1 | 0.004976 |
| CYP3A4 | 0.004974 |
| FOXJ3 | 0.004973 |
| LSM11 | 0.004972 |
| RSC1A1 | 0.004971 |
| PLEKHG3 | 0.004971 |
| CEP44 | 0.004970 |
| PHKA1 | 0.004969 |
| NBPF1 | 0.004967 |
| LRRIQ3 | 0.004965 |
| SMG1 | 0.004963 |
| ZNF428 | 0.004960 |
| RASL11B | 0.004957 |
| ZSCAN26 | 0.004953 |
| NBPF6 | 0.004950 |
| HP1BP3 | 0.004950 |
| MPZ | 0.004947 |
| MTX3 | 0.004947 |
| SEH1L | 0.004946 |
| KLHL15 | 0.004946 |
| KCTD3 | 0.004945 |
| SARS2 | 0.004944 |
| ZNF449 | 0.004943 |
| PRMT6 | 0.004940 |
| OR56B4 | 0.004940 |
| RNGTT | 0.004937 |
| ULK1 | 0.004936 |
| GSE1 | 0.004933 |
| MYH3 | 0.004933 |
| TIAM2 | 0.004925 |
| HES4 | 0.004924 |
| KNOP1 | 0.004924 |
| ZNF678 | 0.004923 |
| PLEKHJ1 | 0.004922 |
| CCDC85C | 0.004922 |
| ZNF346 | 0.004921 |
| F13B | 0.004920 |
| ZNF585A | 0.004919 |
| ABTB2 | 0.004919 |
| MTERF1 | 0.004919 |
| ZNF721 | 0.004918 |
| TENM2 | 0.004914 |
| CAMSAP1 | 0.004913 |
| VWA5B1 | 0.004913 |
| CCDC39 | 0.004911 |
| RNF168 | 0.004910 |
| TTLL8 | 0.004909 |
| SLC25A15 | 0.004909 |
| PLOD2 | 0.004909 |
| TRPC7 | 0.004907 |
| HUS1B | 0.004907 |
| CEP57 | 0.004903 |
| PMAIP1 | 0.004903 |
| ZMYM3 | 0.004902 |
| CSE1L | 0.004901 |
| TCL1B | 0.004900 |
| TERFI | 0.004899 |
| LPO | 0.004899 |
| MBL2 | 0.004898 |
| LRIT3 | 0.004898 |
| USP20 | 0.004897 |
| C9orf142 | 0.004896 |
| TUBB | 0.004895 |
| SLC25A36 | 0.004893 |
| COQ3 | 0.004893 |
| TDRD5 | 0.004889 |
| RCC1 | 0.004888 |
| EXOSC3 | 0.004888 |
| TSPYL5 | 0.004884 |
| NAT14 | 0.004884 |
| ZNF786 | 0.004884 |
| HNRNPA2B1 | 0.004883 |
| TYW5 | 0.004882 |
| ZNF484 | 0.004880 |
| ZNF821 | 0.004880 |
| PFKFB1 | 0.004877 |
| POTEG | 0.004876 |
| ZNF256 | 0.004876 |
| SENP7 | 0.004876 |
| RBM28 | 0.004876 |
| ARID2 | 0.004875 |
| C2orf72 | 0.004874 |
| ZNF536 | 0.004873 |
| SLC7A5 | 0.004873 |
| ZNF48 | 0.004873 |
| MAGOHB | 0.004871 |
| HERC2 | 0.004870 |
| METTL22 | 0.004867 |
| CUL9 | 0.004865 |
| NBPF12 | 0.004864 |
| TCP10 | 0.004863 |
| SUV39H1 | 0.004862 |
| RBM33 | 0.004861 |
| KCTD13 | 0.004860 |
| GABRD | 0.004860 |
| SPRR2F | 0.004859 |
| GRPEL2 | 0.004858 |
| PLXNB1 | 0.004858 |
| GOLGA6B | 0.004858 |
| GCNT7 | 0.004857 |
| METTL12 | 0.004857 |
| USP31 | 0.004856 |
| EEF1E1 | 0.004847 |
| SMC6 | 0.004846 |
| OLIG3 | 0.004839 |
| KIF7 | 0.004839 |
| ACTRT1 | 0.004839 |
| UBA2 | 0.004839 |
| PRPF4B | 0.004838 |
| KLHL42 | 0.004838 |
| CSH2 | 0.004836 |
| TCF12 | 0.004835 |
| VBP1 | 0.004834 |
| PLCD4 | 0.004833 |
| TDRD15 | 0.004831 |
| CPN2 | 0.004829 |
| ARL6IP6 | 0.004829 |
| PLEKHA8 | 0.004827 |
| CCNT2 | 0.004825 |
| KIAA1468 | 0.004824 |
| FBX032 | 0.004822 |
| OR51T1 | 0.004821 |
| ATR | 0.004821 |
| UBE3D | 0.004819 |
| PANK1 | 0.004818 |
| IPO9 | 0.004815 |
| TTI1 | 0.004813 |
| DDIT3 | 0.004812 |
| SMIM8 | 0.004812 |
| ZNF202 | 0.004811 |
| SUDS3 | 0.004811 |
| SLC17A8 | 0.004810 |
| TPTE2 | 0.004803 |
| KMT2B | 0.004801 |
| PIGW | 0.004800 |
| SPATA1 | 0.004800 |
| ZNF573 | 0.004799 |
| SH2D6 | 0.004798 |
| REPIN1 | 0.004798 |
| DCK | 0.004798 |
| SVIP | 0.004796 |
| HHIPL2 | 0.004795 |
| TMEM171 | 0.004795 |
| HTR1E | 0.004795 |
| AHRR | 0.004794 |
| SLC10A5 | 0.004792 |
| ANKRD33 | 0.004792 |
| ZNF485 | 0.004791 |
| KRTAP5-4 | 0.004791 |
| RLF | 0.004791 |
| HAPLN4 | 0.004790 |
| GLRA3 | 0.004789 |
| PUS7 | 0.004788 |
| ZNF579 | 0.004788 |
| SPDYE2B | 0.004787 |
| HRG | 0.004786 |
| PATE3 | 0.004783 |
| ZNF653 | 0.004781 |
| RCOR3 | 0.004781 |
| IAPP | 0.004780 |
| CEP162 | 0.004778 |
| GDA | 0.004778 |
| PPP1R3A | 0.004776 |
| SAFB2 | 0.004776 |
| AARS2 | 0.004775 |
| RBBP8 | 0.004771 |
| EXOG | 0.004770 |
| FAM132A | 0.004769 |
| CEP85L | 0.004769 |
| JAG2 | 0.004767 |
| C1orf131 | 0.004766 |
| DIO2 | 0.004766 |
| CROCC | 0.004765 |
| LGSN | 0.004763 |
| PLEKHG4 | 0.004763 |
| RAD21 | 0.004763 |
| DCLRE1A | 0.004762 |
| TARBP1 | 0.004761 |
| PAX2 | 0.004760 |
| NFXL1 | 0.004760 |
| RNF150 | 0.004759 |
| FKBP4 | 0.004757 |
| PEX1 | 0.004756 |
| ZNF23 | 0.004753 |
| RTP1 | 0.004752 |
| ZBTB5 | 0.004751 |
| PLIN1 | 0.004747 |
| HPSE | 0.004745 |
| TRERF1 | 0.004744 |
| ZNF320 | 0.004744 |
| GALP | 0.004743 |
| CDK5RAP2 | 0.004742 |
| KRBA1 | 0.004741 |
| CXXC1 | 0.004740 |
| RNF222 | 0.004740 |
| KCND3 | 0.004738 |
| SH3BP4 | 0.004737 |
| PRRT3 | 0.004737 |
| SMYD3 | 0.004736 |
| WDR5 | 0.004734 |
| KIF3A | 0.004733 |
| CCDC88C | 0.004733 |
| KCNK17 | 0.004733 |
| THUMPD2 | 0.004731 |
| F7 | 0.004731 |
| OR11H7 | 0.004730 |
| GRIN2B | 0.004726 |
| KMT2A | 0.004724 |
| DUS1L | 0.004723 |
| SMARCAD1 | 0.004723 |
| SPAST | 0.004723 |
| GRID2IP | 0.004722 |
| TIMM44 | 0.004720 |
| DENND5B | 0.004718 |
| IRGQ | 0.004717 |
| FAM200B | 0.004716 |
| PGP | 0.004714 |
| IL36G | 0.004714 |
| C16orf92 | 0.004712 |
| AGMAT | 0.004712 |
| MUC17 | 0.004711 |
| ZKSCAN4 | 0.004710 |
| PLXDC1 | 0.004710 |
| GIF | 0.004709 |
| NPAT | 0.004709 |
| P2RX3 | 0.004708 |
| CCDC167 | 0.004708 |
| DFNB59 | 0.004707 |
| GPATCH1 | 0.004706 |
| IFRD1 | 0.004702 |
| OSGEP | 0.004701 |
| TCHP | 0.004700 |
| LVRN | 0.004699 |
| TTLL5 | 0.004698 |
| GRID2 | 0.004698 |
| TEX38 | 0.004696 |
| ZZZ3 | 0.004694 |
| TBCCD1 | 0.004694 |
| CBX3 | 0.004692 |
| MDH1B | 0.004692 |
| CPN1 | 0.004692 |
| GRIPAP1 | 0.004690 |
| DARS2 | 0.004690 |
| EIF4E1B | 0.004689 |
| SALL2 | 0.004689 |
| SLC25A10 | 0.004688 |
| NOXO1 | 0.004688 |
| AGK | 0.004687 |
| NODAL | 0.004687 |
| FGF11 | 0.004686 |
| ZNF570 | 0.004685 |
| ZNF234 | 0.004681 |
| KAAG1 | 0.004679 |
| SLITRK6 | 0.004678 |
| WDR5B | 0.004678 |
| DNTT | 0.004677 |
| NEDD1 | 0.004677 |
| PPP5D1 | 0.004675 |
| ATP2A3 | 0.004675 |
| PITX2 | 0.004674 |
| YEATS4 | 0.004674 |
| GPR139 | 0.004673 |
| S100Z | 0.004673 |
| HIC2 | 0.004673 |
| G6PC | 0.004672 |
| EXOSC9 | 0.004671 |
| PRIMA1 | 0.004671 |
| MTURN | 0.004670 |
| MORN4 | 0.004670 |
| HNRNPR | 0.004669 |
| C19orf67 | 0.004669 |
| ADGRA3 | 0.004669 |
| DCAF13 | 0.004665 |
| ARL16 | 0.004664 |
| KCTD7 | 0.004664 |
| IQCJ | 0.004663 |
| NDE1 | 0.004660 |
| TRMT1 | 0.004660 |
| RBM20 | 0.004659 |
| FAM205A | 0.004656 |
| CCDC66 | 0.004654 |
| ZNF805 | 0.004654 |
| VARS2 | 0.004653 |
| GPR156 | 0.004653 |
| PHKG1 | 0.004651 |
| HSD11B2 | 0.004651 |
| HCRTR1 | 0.004650 |
| TMEM234 | 0.004650 |
| ACVR2A | 0.004649 |
| HSPD1 | 0.004649 |
| YOD1 | 0.004649 |
| GDPD1 | 0.004649 |
| GPR149 | 0.004647 |
| LHFPL1 | 0.004646 |
| PON1 | 0.004644 |
| ADAT2 | 0.004644 |
| TUBGCP3 | 0.004641 |
| APAF1 | 0.004641 |
| ZNF562 | 0.004639 |
| CAND1 | 0.004638 |
| LMBRD2 | 0.004637 |
| GGCT | 0.004633 |
| DPH2 | 0.004632 |
| LSG1 | 0.004630 |
| PANK3 | 0.004629 |
| ZNF169 | 0.004629 |
| KDM3A | 0.004627 |
| USP48 | 0.004626 |
| SLC17A7 | 0.004626 |
| CC2D1A | 0.004624 |
| THPO | 0.004623 |
| SNRPE | 0.004623 |
| MMP21 | 0.004621 |
| OFD1 | 0.004620 |
| JARID2 | 0.004619 |
| CEP164 | 0.004619 |
| ADRA2B | 0.004619 |
| TOX | 0.004617 |
| TXNRD3NB | 0.004617 |
| GOLGA6D | 0.004615 |
| ZNF284 | 0.004615 |
| WISP3 | 0.004614 |
| SLC10A1 | 0.004612 |
| HN1 | 0.004612 |
| KIAA0586 | 0.004612 |
| ZNF699 | 0.004610 |
| RNF224 | 0.004608 |
| DDX51 | 0.004606 |
| SMOC2 | 0.004602 |
| THOC3 | 0.004602 |
| CCKAR | 0.004602 |
| DLG3 | 0.004595 |
| SLC22A1 | 0.004594 |
| CCDC24 | 0.004592 |
| XXYLT1 | 0.004591 |
| TRAPPC2 | 0.004591 |
| OPN5 | 0.004590 |
| EXTL3 | 0.004590 |
| CCAR1 | 0.004589 |
| IGLON5 | 0.004589 |
| TMEM210 | 0.004588 |
| C5orf63 | 0.004588 |
| TRMT10B | 0.004586 |
| COX7B2 | 0.004586 |
| GLB1L3 | 0.004586 |
| PIAS4 | 0.004586 |
| SERPINI1 | 0.004583 |
| SNRPA1 | 0.004583 |
| HINFP | 0.004582 |
| FAM3B | 0.004581 |
| IFT80 | 0.004578 |
| PAQR3 | 0.004578 |
| ODF3 | 0.004577 |
| NUPL2 | 0.004576 |
| KCNA2 | 0.004576 |
| LIN7B | 0.004575 |
| ZSCAN21 | 0.004574 |
| ZBTB18 | 0.004573 |
| CMC2 | 0.004573 |
| ZNF7 | 0.004570 |
| ACBD6 | 0.004568 |
| ADAM7 | 0.004566 |
| NR1I3 | 0.004565 |
| TEFM | 0.004565 |
| ABCA2 | 0.004564 |
| FGF22 | 0.004563 |
| FARSB | 0.004562 |
| POTEH | 0.004561 |
| WRN | 0.004561 |
| GAGE1 | 0.004561 |
| OTUD6B | 0.004560 |
| SGSM1 | 0.004557 |
| HSPA1A | 0.004556 |
| GPATCH2 | 0.004554 |
| PCSK4 | 0.004553 |
| CSRP2 | 0.004553 |
| C1orf100 | 0.004551 |
| KBTBD6 | 0.004550 |
| ACIN1 | 0.004543 |
| ZNF347 | 0.004542 |
| PPIF | 0.004542 |
| ATG4B | 0.004541 |
| TNKS | 0.004541 |
| GTF3C5 | 0.004541 |
| FAM171A1 | 0.004540 |
| GCLC | 0.004539 |
| BAZ2B | 0.004539 |
| DDHD2 | 0.004539 |
| CASP2 | 0.004539 |
| CACNG7 | 0.004537 |
| GRM3 | 0.004537 |
| WDR12 | 0.004537 |
| CEP290 | 0.004534 |
| KLHDC9 | 0.004534 |
| HS3ST4 | 0.004529 |
| BOC | 0.004529 |
| CYP3A43 | 0.004529 |
| C19orf73 | 0.004529 |
| CENPC | 0.004528 |
| FAM71C | 0.004527 |
| POLD3 | 0.004526 |
| TCEANC2 | 0.004526 |
| PLCXD1 | 0.004526 |
| CYP26C1 | 0.004526 |
| PDRG1 | 0.004525 |
| SF3A2 | 0.004525 |
| NLRP1 | 0.004523 |
| DDHD1 | 0.004521 |
| NLRP5 | 0.004521 |
| GBA2 | 0.004518 |
| ALG10 | 0.004517 |
| ZNF627 | 0.004517 |
| TAF6L | 0.004516 |
| ABI2 | 0.004515 |
| CDRT15 | 0.004515 |
| HDAC9 | 0.004515 |
| ASTL | 0.004514 |
| LIN37 | 0.004513 |
| STX16 | 0.004512 |
| TMEM161B | 0.004509 |
| ERI2 | 0.004507 |
| PCDH11X | 0.004504 |
| PDCD5 | 0.004503 |
| UBR5 | 0.004501 |
| SLC22A13 | 0.004497 |
| BPIFC | 0.004496 |
| LIN54 | 0.004493 |
| SLC30A2 | 0.004488 |
| DUT | 0.004488 |
| MUM1 | 0.004487 |
| P2RY4 | 0.004485 |
| ADPRHL1 | 0.004484 |
| UNCX | 0.004483 |
| FEM1A | 0.004479 |
| NT5C1B | 0.004478 |
| COLQ | 0.004478 |
| OR8A1 | 0.004477 |
| GJA9 | 0.004477 |
| RASA2 | 0.004476 |
| BCL7A | 0.004475 |
| TRAPPC5 | 0.004475 |
| HDX | 0.004475 |
| WDYHV1 | 0.004475 |
| STYK1 | 0.004472 |
| SKOR2 | 0.004469 |
| TMEM160 | 0.004468 |
| ZNF71 | 0.004466 |
| BOD1 | 0.004466 |
| ZNF558 | 0.004464 |
| TMEM38B | 0.004461 |
| PCYOX1L | 0.004461 |
| ZNF517 | 0.004461 |
| SHANK1 | 0.004459 |
| PRB2 | 0.004459 |
| LAGE3 | 0.004458 |
| MIB1 | 0.004456 |
| TADA1 | 0.004455 |
| ZMIZ2 | 0.004455 |
| DISC1 | 0.004454 |
| FBN3 | 0.004453 |
| FAM83B | 0.004452 |
| ZNF76 | 0.004449 |
| EXTL2 | 0.004449 |
| SPZ1 | 0.004448 |
| EPOR | 0.004446 |
| NIN | 0.004445 |
| OR7D2 | 0.004445 |
| RNF34 | 0.004445 |
| IGFBP5 | 0.004444 |
| F8A3 | 0.004443 |
| PHF20L1 | 0.004442 |
| ZNF430 | 0.004442 |
| VAX1 | 0.004441 |
| PPT2 | 0.004441 |
| TIMM8A | 0.004440 |
| PLP1 | 0.004439 |
| SLC25A40 | 0.004439 |
| RAB30 | 0.004439 |
| SOX9 | 0.004438 |
| CCDC116 | 0.004437 |
| BCS1L | 0.004436 |
| ZNF211 | 0.004435 |
| NGEF | 0.004434 |
| SERPINA7 | 0.004433 |
| NELFCD | 0.004433 |
| ICE1 | 0.004432 |
| RAB33A | 0.004432 |
| DPY19L3 | 0.004432 |
| OR2B2 | 0.004431 |
| LMBR1L | 0.004431 |
| MAPK4 | 0.004430 |
| IMPG1 | 0.004430 |
| NUDT1 | 0.004430 |
| TUB | 0.004429 |
| FRS3 | 0.004428 |
| SBNO1 | 0.004427 |
| REEP1 | 0.004427 |
| C9orf72 | 0.004426 |
| LNX1 | 0.004425 |
| RNF219 | 0.004420 |
| ZUFSP | 0.004419 |
| CACYBP | 0.004419 |
| TIAF1 | 0.004417 |
| ZNF14 | 0.004416 |
| SULT2A1 | 0.004416 |
| RPP21 | 0.004416 |
| ADAM2 | 0.004415 |
| DCAF15 | 0.004415 |
| QRFP | 0.004415 |
| PPME1 | 0.004414 |
| TRPV5 | 0.004414 |
| DCLK1 | 0.004412 |
| ETNK2 | 0.004410 |
| LTN1 | 0.004410 |
| SAG | 0.004407 |
| KPNA5 | 0.004406 |
| OPTC | 0.004404 |
| C9orf129 | 0.004404 |
| HRASLS | 0.004403 |
| RC3H2 | 0.004403 |
| AP4M1 | 0.004402 |
| VPS8 | 0.004400 |
| SLC22A17 | 0.004400 |
| TAZ | 0.004399 |
| ENOSF1 | 0.004398 |
| ZNF354C | 0.004398 |
| ESCO1 | 0.004397 |
| MBNL3 | 0.004397 |
| SLC5A3 | 0.004396 |
| HELB | 0.004396 |
| LECT2 | 0.004396 |
| LRRC8B | 0.004396 |
| KDM1B | 0.004395 |
| OR1J1 | 0.004394 |
| LRRC34 | 0.004394 |
| FXR1 | 0.004393 |
| PLAC1 | 0.004393 |
| HAO2 | 0.004393 |
| RSRC2 | 0.004391 |
| GPRC5B | 0.004391 |
| UBE2E3 | 0.004390 |
| MCHR2 | 0.004388 |
| AWAT1 | 0.004388 |
| GPR89B | 0.004387 |
| TUBGCP4 | 0.004385 |
| FRG2 | 0.004385 |
| ELF2 | 0.004384 |
| ZNF534 | 0.004384 |
| CCDC84 | 0.004382 |
| LDHAL6A | 0.004381 |
| RGSL1 | 0.004380 |
| HSPA1B | 0.004380 |
| CEP19 | 0.004380 |
| TULP1 | 0.004379 |
| GTF2H4 | 0.004377 |
| ODF2L | 0.004377 |
| PRKAB2 | 0.004377 |
| INTS2 | 0.004377 |
| ATP7B | 0.004376 |
| IRGC | 0.004374 |
| UGT2B11 | 0.004373 |
| STH | 0.004373 |
| FOXD4 | 0.004373 |
| STK32C | 0.004372 |
| DAZ2 | 0.004371 |
| ORC2 | 0.004371 |
| FASTKD3 | 0.004371 |
| SYNE2 | 0.004370 |
| C9orf153 | 0.004370 |
| ZNF316 | 0.004370 |
| NDOR1 | 0.004370 |
| CRTC1 | 0.004369 |
| PDDC1 | 0.004368 |
| SLBP | 0.004365 |
| PTPRS | 0.004364 |
| ZNF17 | 0.004364 |
| U2AF1L4 | 0.004364 |
| TRIM62 | 0.004364 |
| HMG20B | 0.004363 |
| NR2C2AP | 0.004361 |
| ZNF664 | 0.004361 |
| ZFP90 | 0.004359 |
| MAGEF1 | 0.004357 |
| LEMD2 | 0.004355 |
| DUS4L | 0.004354 |
| FSCN2 | 0.004354 |
| TTYH3 | 0.004352 |
| C8orf37 | 0.004351 |
| PROB1 | 0.004349 |
| TMEM61 | 0.004348 |
| PDF | 0.004348 |
| FRMD7 | 0.004348 |
| FAF1 | 0.004347 |
| ZNF512B | 0.004346 |
| MTG1 | 0.004346 |
| ID4 | 0.004344 |
| PDC | 0.004342 |
| SERF1A | 0.004342 |
| TELO2 | 0.004341 |
| RNF32 | 0.004341 |
| KLHL29 | 0.004340 |
| TRHR | 0.004340 |
| DNAJC24 | 0.004340 |
| EFCAB11 | 0.004335 |
| COLCA2 | 0.004335 |
| TMEM78 | 0.004334 |
| PPP1R16A | 0.004332 |
| RALGPS2 | 0.004330 |
| UNC5CL | 0.004330 |
| GYS2 | 0.004330 |
| NABP2 | 0.004329 |
| ANKRD13D | 0.004328 |
| PPM1L | 0.004327 |
| PPOX | 0.004327 |
| GNL3L | 0.004327 |
| KLHL34 | 0.004325 |
| GRAMD1B | 0.004325 |
| ZNF638 | 0.004325 |
| GSX2 | 0.004324 |
| CNKSR2 | 0.004323 |
| TBC1D26 | 0.004322 |
| FNBP1L | 0.004321 |
| SFXN5 | 0.004319 |
| CTAG2 | 0.004317 |
| C14orf37 | 0.004317 |
| ILKAP | 0.004314 |
| ZNF33A | 0.004314 |
| ELAVL1 | 0.004313 |
| PCID2 | 0.004312 |
| SCUBE3 | 0.004310 |
| GOLGA6L22 | 0.004309 |
| TAF9B | 0.004309 |
| C21orf59 | 0.004309 |
| TRIM17 | 0.004307 |
| CDRT4 | 0.004306 |
| GAMT | 0.004305 |
| FADS2 | 0.004304 |
| FAM168A | 0.004304 |
| SMARCA4 | 0.004303 |
| MCM5 | 0.004302 |
| TEX10 | 0.004302 |
| C3orf20 | 0.004301 |
| SETD4 | 0.004301 |
| MSL2 | 0.004299 |
| TTL | 0.004294 |
| ZNF862 | 0.004293 |
| AJAP1 | 0.004291 |
| HNRNPDL | 0.004290 |
| ZNF764 | 0.004290 |
| CLDN11 | 0.004289 |
| TRPM3 | 0.004288 |
| TPGS1 | 0.004287 |
| NPIPB3 | 0.004287 |
| HTR4 | 0.004282 |
| TMEM5 | 0.004282 |
| KIAA1644 | 0.004281 |
| ZFP69 | 0.004279 |
| POLR3A | 0.004278 |
| FBXL19 | 0.004277 |
| TRIB2 | 0.004275 |
| C12orf65 | 0.004275 |
| POLRMT | 0.004274 |
| EXOC5 | 0.004273 |
| TMPRSS6 | 0.004273 |
| SH3GLB2 | 0.004272 |
| PPP5C | 0.004272 |
| KIAA0753 | 0.004271 |
| ZDHHC21 | 0.004270 |
| TMEM121 | 0.004269 |
| TREX2 | 0.004265 |
| TSPY3 | 0.004264 |
| TBX19 | 0.004263 |
| GPRASP2 | 0.004262 |
| NME1 | 0.004261 |
| DCAF17 | 0.004260 |
| RANBP17 | 0.004260 |
| RAB2B | 0.004259 |
| GPR35 | 0.004259 |
| BLMH | 0.004257 |
| ZNF343 | 0.004255 |
| DNAAF2 | 0.004255 |
| U2AF2 | 0.004255 |
| LRP1B | 0.004254 |
| RNASEH2C | 0.004252 |
| C15orf62 | 0.004252 |
| AHCTF1 | 0.004252 |
| PHLDB3 | 0.004251 |
| SMC01 | 0.004251 |
| HENMT1 | 0.004251 |
| APOC3 | 0.004249 |
| ZNF230 | 0.004248 |
| WDR35 | 0.004247 |
| TSTD2 | 0.004245 |
| PFAS | 0.004244 |
| GPR45 | 0.004244 |
| CCZ1B | 0.004242 |
| ZNF827 | 0.004241 |
| OR13G1 | 0.004241 |
| TAS2R43 | 0.004241 |
| CHRND | 0.004240 |
| ZNF24 | 0.004240 |
| ZNF540 | 0.004239 |
| NCAPH2 | 0.004239 |
| KIAA1958 | 0.004238 |
| TCP1 | 0.004238 |
| ORAOV1 | 0.004238 |
| CPSF7 | 0.004237 |
| ABCB9 | 0.004236 |
| D2HGDH | 0.004236 |
| OR1Q1 | 0.004235 |
| ADNP | 0.004235 |
| MSANTD1 | 0.004234 |
| HRAS | 0.004233 |
| SEPSECS | 0.004233 |
| FAAP100 | 0.004229 |
| GLI4 | 0.004229 |
| ZNF30 | 0.004227 |
| BAMB1 | 0.004226 |
| ZNF101 | 0.004226 |
| KHK | 0.004225 |
| PSMG1 | 0.004223 |
| TKTL2 | 0.004223 |
| OTOS | 0.004223 |
| CHRD | 0.004222 |
| LRP4 | 0.004220 |
| LIPE | 0.004220 |
| NVL | 0.004220 |
| OR51H1 | 0.004218 |
| FBXO10 | 0.004218 |
| KLHL3 | 0.004215 |
| CACNA2D3 | 0.004215 |
| CACTIN | 0.004213 |
| PLCH2 | 0.004212 |
| WDR53 | 0.004212 |
| S100A7A | 0.004208 |
| MED14 | 0.004207 |
| SUCO | 0.004206 |
| DGKZ | 0.004205 |
| MAU2 | 0.004204 |
| ADGRB3 | 0.004204 |
| SRSF6 | 0.004202 |
| MADD | 0.004201 |
| ZNF614 | 0.004201 |
| PHTF1 | 0.004194 |
| PACS2 | 0.004193 |
| GRK4 | 0.004193 |
| CEP63 | 0.004192 |
| LSM4 | 0.004192 |
| NT5DC2 | 0.004191 |
| HNRNPD | 0.004191 |
| CEP70 | 0.004190 |
| MRPL42 | 0.004189 |
| DNHD1 | 0.004189 |
| OR51G2 | 0.004189 |
| OCRL | 0.004187 |
| BRAT1 | 0.004185 |
| CREG2 | 0.004183 |
| HTATSF1 | 0.004182 |
| FRAS1 | 0.004182 |
| OTULIN | 0.004181 |
| EIF4A2 | 0.004180 |
| FBRSL1 | 0.004179 |
| KIAA1147 | 0.004178 |
| SLC9B2 | 0.004177 |
| MFN1 | 0.004176 |
| TP53BP1 | 0.004176 |
| SERPINE2 | 0.004172 |
| SERF1B | 0.004171 |
| MTHFD1 | 0.004170 |
| IGFL1 | 0.004170 |
| TXNL4A | 0.004169 |
| KLHDC4 | 0.004166 |
| FANCE | 0.004164 |
| SOX14 | 0.004163 |
| ZXDC | 0.004163 |
| SEZ6L2 | 0.004162 |
| FAAH | 0.004160 |
| MED23 | 0.004159 |
| NAT9 | 0.004158 |
| MTOR | 0.004158 |
| USP42 | 0.004157 |
| THBS3 | 0.004157 |
| OR6F1 | 0.004155 |
| SP6 | 0.004154 |
| ME3 | 0.004153 |
| DUX4 | 0.004152 |
| RGPD4 | 0.004150 |
| TIMM21 | 0.004150 |
| TARS | 0.004149 |
| C9orf47 | 0.004149 |
| ABHD10 | 0.004147 |
| MAPK11 | 0.004147 |
| MUS81 | 0.004145 |
| USP35 | 0.004145 |
| CCNG2 | 0.004145 |
| ZBED4 | 0.004145 |
| FAAP24 | 0.004144 |
| NUP62 | 0.004141 |
| SLIRP | 0.004141 |
| CORO6 | 0.004140 |
| PIN1 | 0.004140 |
| TAF11 | 0.004140 |
| H2AFV | 0.004135 |
| SRP9 | 0.004135 |
| TRIO | 0.004132 |
| PHLPP2 | 0.004132 |
| TRIM24 | 0.004131 |
| ZNF701 | 0.004130 |
| SERPINC1 | 0.004130 |
| ANKEF1 | 0.004130 |
| AGO1 | 0.004129 |
| RUSC1 | 0.004129 |
| FUS | 0.004128 |
| RAD51D | 0.004126 |
| RBM10 | 0.004126 |
| KRTAP1-3 | 0.004124 |
| ZNF37A | 0.004122 |
| RAD1 | 0.004121 |
| EPHB2 | 0.004120 |
| PRMT3 | 0.004118 |
| EXOSC10 | 0.004117 |
| DERL3 | 0.004115 |
| CCNB1IP1 | 0.004114 |
| POLR3F | 0.004112 |
| TBL1XR1 | 0.004110 |
| CTDSPL2 | 0.004110 |
| MGA | 0.004109 |
| NAE1 | 0.004106 |
| PRKRIP1 | 0.004106 |
| APBA1 | 0.004105 |
| PMP2 | 0.004104 |
| BCL9 | 0.004104 |
| EIF2B5 | 0.004104 |
| TRIM27 | 0.004103 |
| MITD1 | 0.004102 |
| MLH3 | 0.004098 |
| POT1 | 0.004098 |
| CCL27 | 0.004098 |
| GPR89A | 0.004098 |
| SLCO1B1 | 0.004097 |
| NLK | 0.004096 |
| H2AFY | 0.004095 |
| OMP | 0.004095 |
| ZKSCAN3 | 0.004094 |
| PDE10A | 0.004093 |
| MTCP1 | 0.004093 |
| DZIP3 | 0.004090 |
| GOLGA8K | 0.004090 |
| PTPRD | 0.004090 |
| SMG9 | 0.004088 |
| ZNF45 | 0.004087 |
| THOC2 | 0.004086 |
| PCDH11Y | 0.004085 |
| ALG13 | 0.004083 |
| GAN | 0.004083 |
| UPK3BL | 0.004083 |
| TMEM88B | 0.004082 |
| SPAG16 | 0.004082 |
| ENGASE | 0.004082 |
| AMN1 | 0.004079 |
| SPDYE5 | 0.004079 |
| ZNF766 | 0.004079 |
| COLEC11 | 0.004076 |
| ARHGEF16 | 0.004075 |
| KIF1B | 0.004075 |
| ZNF836 | 0.004073 |
| PKNOX1 | 0.004073 |
| THAP10 | 0.004072 |
| HAO1 | 0.004072 |
| KDM5B | 0.004072 |
| BPTF | 0.004072 |
| NFYA | 0.004071 |
| LCLAT1 | 0.004071 |
| SBSN | 0.004069 |
| HIST1H1A | 0.004069 |
| DHX35 | 0.004068 |
| TAF1 | 0.004068 |
| OR2T8 | 0.004066 |
| GART | 0.004066 |
| SETD5 | 0.004065 |
| ASB5 | 0.004062 |
| HMGXB4 | 0.004062 |
| ARHGEF19 | 0.004062 |
| F9 | 0.004061 |
| EYA3 | 0.004060 |
| CHAC2 | 0.004059 |
| RAPSN | 0.004058 |
| SMN2 | 0.004058 |
| BRD9 | 0.004057 |
| ZNF565 | 0.004057 |
| SGSM2 | 0.004056 |
| SNRPA | 0.004055 |
| HNRNPH3 | 0.004054 |
| RRAGB | 0.004053 |
| ENAH | 0.004052 |
| CEP295NL | 0.004051 |
| FAM120C | 0.004050 |
| HNRNPA3 | 0.004048 |
| MEX3C | 0.004047 |
| TTPAL | 0.004047 |
| AVL9 | 0.004047 |
| ZNF630 | 0.004047 |
| TMEM237 | 0.004046 |
| TMPRSS3 | 0.004044 |
| DVL2 | 0.004043 |
| HOXA1 | 0.004043 |
| LPIN1 | 0.004042 |
| FAM208B | 0.004040 |
| LSM5 | 0.004040 |
| NDUFAF7 | 0.004039 |
| PSPC1 | 0.004038 |
| SNRNP25 | 0.004038 |
| C1QTNF9B | 0.004037 |
| NUCKS1 | 0.004034 |
| TBCB | 0.004031 |
| ACER2 | 0.004030 |
| NMBR | 0.004029 |
| SUGP1 | 0.004028 |
| LIPJ | 0.004026 |
| BIVM | 0.004026 |
| SMLR1 | 0.004025 |
| MRGBP | 0.004025 |
| GLRX5 | 0.004025 |
| MLIP | 0.004024 |
| WDR87 | 0.004024 |
| MDN1 | 0.004021 |
| KIAA1324 | 0.004021 |
| TMEM259 | 0.004020 |
| STAG1 | 0.004017 |
| C14orf79 | 0.004017 |
| NLGN2 | 0.004017 |
| HAUS7 | 0.004015 |
| GPR3 | 0.004015 |
| TDRD6 | 0.004011 |
| FMR1 | 0.004010 |
| ZNF451 | 0.004010 |
| ZNF548 | 0.004006 |
| PWWP2A | 0.004005 |
| MED17 | 0.004005 |
| RPP40 | 0.004002 |
| CAMTA1 | 0.004000 |
| ZNF615 | 0.003999 |
| CNTRL | 0.003999 |
| ZNF181 | 0.003999 |
| NRDE2 | 0.003998 |
| LRRC58 | 0.003998 |
| AKT2 | 0.003998 |
| CSNK1E | 0.003997 |
| DHX36 | 0.003996 |
| PRSS2 | 0.003995 |
| ENOPH1 | 0.003995 |
| CCDC93 | 0.003995 |
| HTR2C | 0.003995 |
| FAM122C | 0.003994 |
| TFDP2 | 0.003994 |
| SRSF7 | 0.003994 |
| ZNF684 | 0.003994 |
| DOCK7 | 0.003993 |
| SH2B1 | 0.003992 |
| C7orf73 | 0.003992 |
| ABHD3 | 0.003989 |
| EID2 | 0.003987 |
| ZNF446 | 0.003986 |
| SSBP3 | 0.003986 |
| MAP2K6 | 0.003986 |
| CEACAM7 | 0.003986 |
| ZNF639 | 0.003986 |
| STK25 | 0.003985 |
| ZNF383 | 0.003984 |
| RBM26 | 0.003984 |
| BANP | 0.003982 |
| UHRF1BP1 | 0.003980 |
| CMSS1 | 0.003979 |
| ZXDA | 0.003978 |
| CCDC140 | 0.003976 |
| GLYAT | 0.003975 |
| RUNX1T1 | 0.003975 |
| ASB4 | 0.003973 |
| ANKRD6 | 0.003973 |
| FUT5 | 0.003973 |
| ZCCHC8 | 0.003973 |
| UQCC1 | 0.003971 |
| OBSL1 | 0.003971 |
| DLG2 | 0.003969 |
| TESK2 | 0.003969 |
| ACTL7B | 0.003968 |
| FAM219A | 0.003967 |
| KDM2B | 0.003965 |
| MTERF3 | 0.003965 |
| NPIPB15 | 0.003963 |
| GLT1D1 | 0.003962 |
| C14orf93 | 0.003962 |
| FAM71E1 | 0.003960 |
| KMT2C | 0.003958 |
| ATXN3 | 0.003958 |
| ZNF665 | 0.003958 |
| ANKRD39 | 0.003957 |
| C3orf14 | 0.003957 |
| SLC2A12 | 0.003956 |
| TUBA1B | 0.003955 |
| TNRC6A | 0.003954 |
| MAPKAPK5 | 0.003952 |
| AKNAD1 | 0.003952 |
| C11orf53 | 0.003951 |
| C19orf44 | 0.003947 |
| NRBP2 | 0.003947 |
| LRRC8D | 0.003946 |
| RBFA | 0.003946 |
| INTU | 0.003944 |
| YAE1D1 | 0.003941 |
| TJAP1 | 0.003938 |
| VHL | 0.003935 |
| NUDT6 | 0.003935 |
| CLRN1 | 0.003935 |
| HSPE1 | 0.003934 |
| OLA1 | 0.003932 |
| KRTAP16-1 | 0.003932 |
| C6orf201 | 0.003932 |
| REV1 | 0.003930 |
| COIL | 0.003929 |
| HSD17B1 | 0.003928 |
| ZKSCAN2 | 0.003928 |
| LRRC40 | 0.003927 |
| APBA3 | 0.003926 |
| HIST1H1C | 0.003923 |
| SBF1 | 0.003923 |
| HDAC4 | 0.003923 |
| DNAJB8 | 0.003922 |
| USP33 | 0.003922 |
| LIPT2 | 0.003922 |
| HBQ1 | 0.003920 |
| ZNF554 | 0.003919 |
| HPS4 | 0.003918 |
| RFPL3 | 0.003918 |
| CCDC148 | 0.003917 |
| DNAJB11 | 0.003916 |
| FAM46D | 0.003916 |
| C10orf88 | 0.003915 |
| MKS1 | 0.003914 |
| ITGA2B | 0.003913 |
| FAM185A | 0.003912 |
| SPATA31E1 | 0.003911 |
| BAZ1B | 0.003910 |
| ZNF585B | 0.003909 |
| ERCC6L2 | 0.003908 |
| ZSWIM5 | 0.003908 |
| TRPT1 | 0.003908 |
| C10orf113 | 0.003907 |
| NOL8 | 0.003906 |
| PRR27 | 0.003906 |
| MY03B | 0.003906 |
| MFSD9 | 0.003905 |
| IPP | 0.003903 |
| COA1 | 0.003902 |
| MICALL2 | 0.003902 |
| WWOX | 0.003902 |
| CSNK1G2 | 0.003902 |
| ILF2 | 0.003901 |
| MBD1 | 0.003901 |
| C2orf61 | 0.003900 |
| UPK1A | 0.003899 |
| ZNF207 | 0.003897 |
| AGT | 0.003897 |
| IFI27L1 | 0.003896 |
| CECR5 | 0.003895 |
| ZNF490 | 0.003894 |
| HSPBAP1 | 0.003894 |
| SLC39A14 | 0.003893 |
| ZNF99 | 0.003891 |
| ZBTB41 | 0.003890 |
| KLHL24 | 0.003890 |
| GMDS | 0.003889 |
| CDK16 | 0.003888 |
| PHF14 | 0.003885 |
| CTAGE6 | 0.003883 |
| TSPY4 | 0.003883 |
| ATM | 0.003882 |
| RAD18 | 0.003882 |
| ZMYND8 | 0.003881 |
| HSFX1 | 0.003881 |
| PATZ1 | 0.003881 |
| COMMD3 | 0.003879 |
| SKIV2L | 0.003877 |
| ARMC1 | 0.003876 |
| DMWD | 0.003875 |
| TRMT5 | 0.003874 |
| KLHL35 | 0.003874 |
| SLC26A1 | 0.003874 |
| OTOGL | 0.003872 |
| PRSS42 | 0.003872 |
| METTL8 | 0.003870 |
| KLHL28 | 0.003869 |
| PSPN | 0.003868 |
| ZBTB6 | 0.003867 |
| HN1L | 0.003864 |
| TNNI3 | 0.003862 |
| ZNF441 | 0.003862 |
| MTPAP | 0.003861 |
| ONECUT3 | 0.003861 |
| TIMM50 | 0.003861 |
| ZNF599 | 0.003860 |
| RNF207 | 0.003860 |
| FAM133B | 0.003859 |
| LANCL2 | 0.003858 |
| EHMT1 | 0.003858 |
| CARF | 0.003854 |
| SNX16 | 0.003854 |
| ENPP1 | 0.003854 |
| ZNF225 | 0.003854 |
| TRRAP | 0.003852 |
| DCP2 | 0.003851 |
| POC5 | 0.003851 |
| FKTN | 0.003851 |
| VPS45 | 0.003850 |
| PCGF6 | 0.003848 |
| PRR23D1 | 0.003848 |
| TSNAXIP1 | 0.003847 |
| GLB1L2 | 0.003846 |
| ITPRIPL1 | 0.003845 |
| ZNF616 | 0.003844 |
| CABP4 | 0.003843 |
| SIGLEC15 | 0.003842 |
| SRSF5 | 0.003841 |
| GPSM1 | 0.003839 |
| COL19A1 | 0.003838 |
| LDOC1L | 0.003838 |
| ABCC2 | 0.003838 |
| TBC1D30 | 0.003835 |
| HIST1H4F | 0.003835 |
| ANAPC7 | 0.003834 |
| KCTD6 | 0.003834 |
| NFRKB | 0.003833 |
| TLE6 | 0.003832 |
| NUP35 | 0.003830 |
| ARFGAP1 | 0.003830 |
| CSRNP2 | 0.003829 |
| CDK11A | 0.003827 |
| DAZAP1 | 0.003826 |
| AFM | 0.003825 |
| NUAK1 | 0.003823 |
| TPRKB | 0.003822 |
| UGT2B10 | 0.003821 |
| PASD1 | 0.003821 |
| CTNNA3 | 0.003821 |
| ZNF716 | 0.003819 |
| SP3 | 0.003819 |
| DCLRE1C | 0.003818 |
| TIGD5 | 0.003816 |
| OR1C1 | 0.003816 |
| C6orf136 | 0.003813 |
| WDR75 | 0.003813 |
| C3orf70 | 0.003813 |
| ANAPC1 | 0.003812 |
| TAS2R8 | 0.003812 |
| FARP2 | 0.003812 |
| LUC7L2 | 0.003809 |
| FITM1 | 0.003809 |
| LRWD1 | 0.003807 |
| NIPBL | 0.003806 |
| CCDC151 | 0.003805 |
| DYRK1A | 0.003805 |
| ZNF324 | 0.003804 |
| SERPINA12 | 0.003804 |
| ESRP1 | 0.003803 |
| HTN1 | 0.003803 |
| PTCD2 | 0.003802 |
| NUP153 | 0.003801 |
| TRIM33 | 0.003800 |
| ZNF20 | 0.003798 |
| SPAG4 | 0.003797 |
| CNOT3 | 0.003795 |
| CGB2 | 0.003794 |
| NBPF3 | 0.003789 |
| ZNF706 | 0.003788 |
| RHOXF2 | 0.003788 |
| RNMT | 0.003788 |
| SLC25A52 | 0.003787 |
| SFSWAP | 0.003786 |
| PIN4 | 0.003786 |
| CTPS2 | 0.003785 |
| ANAPC5 | 0.003784 |
| RHPN2 | 0.003781 |
| TPR | 0.003780 |
| PRPS2 | 0.003780 |
| INO80E | 0.003780 |
| ZNF80 | 0.003778 |
| FBXO46 | 0.003776 |
| SDHAF3 | 0.003776 |
| PROK1 | 0.003775 |
| PPP1R3F | 0.003775 |
| DENND4B | 0.003774 |
| TMEM65 | 0.003773 |
| SAFB | 0.003773 |
| ZBTB1 | 0.003773 |
| ZBED6CL | 0.003772 |
| ALOX12B | 0.003771 |
| GRHL3 | 0.003771 |
| OR52W1 | 0.003770 |
| SLC25A13 | 0.003769 |
| THAP8 | 0.003768 |
| APPL2 | 0.003768 |
| STRN4 | 0.003767 |
| STAU2 | 0.003767 |
| TIGD2 | 0.003767 |
| SHF | 0.003767 |
| PRDM8 | 0.003766 |
| PRPF4 | 0.003765 |
| NNT | 0.003764 |
| DUS3L | 0.003762 |
| CHST1 | 0.003759 |
| PHF7 | 0.003758 |
| LRIG2 | 0.003758 |
| P3H1 | 0.003757 |
| ZNF790 | 0.003756 |
| BTF3L4 | 0.003756 |
| EPM2AIP1 | 0.003756 |
| RELT | 0.003755 |
| KPNB1 | 0.003755 |
| TRIM28 | 0.003754 |
| DNAJC19 | 0.003753 |
| KRIT1 | 0.003753 |
| RNPS1 | 0.003753 |
| PROKR2 | 0.003752 |
| ZNF578 | 0.003752 |
| IER5L | 0.003751 |
| SOX6 | 0.003747 |
| RWDD2A | 0.003747 |
| KRT9 | 0.003745 |
| UBXN11 | 0.003745 |
| CIB4 | 0.003745 |
| POLR2D | 0.003743 |
| ANKRD63 | 0.003741 |
| NAA35 | 0.003741 |
| CLUL1 | 0.003741 |
| ASPHD2 | 0.003739 |
| RECQL5 | 0.003736 |
| ZDHHC11 | 0.003736 |
| EGR4 | 0.003735 |
| GRB10 | 0.003734 |
| PAPOLA | 0.003734 |
| RAVER2 | 0.003732 |
| HCFC1 | 0.003728 |
| GJD4 | 0.003728 |
| RASL10A | 0.003726 |
| GCFC2 | 0.003726 |
| ESPNL | 0.003726 |
| NUP188 | 0.003726 |
| RDH5 | 0.003725 |
| ZNHIT3 | 0.003724 |
| AMY2B | 0.003722 |
| SART3 | 0.003722 |
| NOXA1 | 0.003721 |
| PRR14L | 0.003721 |
| SLFN13 | 0.003719 |
| WDR83 | 0.003719 |
| SMIM13 | 0.003718 |
| PCBP4 | 0.003718 |
| FAM217A | 0.003718 |
| ZNF780A | 0.003716 |
| CRNKL1 | 0.003715 |
| CAGE1 | 0.003715 |
| TIAL1 | 0.003715 |
| DSCR4 | 0.003715 |
| SRSF3 | 0.003715 |
| CRYGN | 0.003715 |
| UTS2B | 0.003714 |
| SPIC | 0.003711 |
| ZFP1 | 0.003711 |
| DCUN1D4 | 0.003709 |
| AUTS2 | 0.003709 |
| PALB2 | 0.003709 |
| ACPT | 0.003708 |
| SNRNP200 | 0.003708 |
| OR51F2 | 0.003708 |
| ALYREF | 0.003706 |
| PANX2 | 0.003706 |
| TGM3 | 0.003706 |
| CDAN1 | 0.003705 |
| ANKLE2 | 0.003705 |
| RAB41 | 0.003705 |
| VSIG8 | 0.003705 |
| BVES | 0.003704 |
| RSF1 | 0.003704 |
| C1orf234 | 0.003703 |
| CDCP2 | 0.003702 |
| GTF2H2 | 0.003701 |
| NBPF26 | 0.003701 |
| ORMDL1 | 0.003701 |
| TRAPPC6B | 0.003700 |
| SPATA5L1 | 0.003700 |
| TXLNG | 0.003699 |
| TCP11L1 | 0.003699 |
| ZNF808 | 0.003699 |
| NEFM | 0.003698 |
| PPIE | 0.003697 |
| REPS1 | 0.003697 |
| ZNF200 | 0.003696 |
| ZNF729 | 0.003696 |
| SLC13A3 | 0.003696 |
| ACAP2 | 0.003696 |
| ARHGEF25 | 0.003695 |
| RPA4 | 0.003694 |
| TAOK1 | 0.003694 |
| TSPY2 | 0.003694 |
| DEFB132 | 0.003691 |
| NSMCE4A | 0.003690 |
| HLCS | 0.003690 |
| CLEC3A | 0.003690 |
| RBM14 | 0.003688 |
| NBPF4 | 0.003688 |
| ARL5B | 0.003687 |
| RHOXF1 | 0.003686 |
| IP6K2 | 0.003685 |
| RFXAP | 0.003683 |
| TYW1B | 0.003683 |
| SIKE1 | 0.003682 |
| SMPD4 | 0.003681 |
| TDRKH | 0.003680 |
| CCT6A | 0.003679 |
| DHX15 | 0.003676 |
| UBE2V2 | 0.003676 |
| ZNF644 | 0.003675 |
| SLC4A7 | 0.003674 |
| MESP2 | 0.003673 |
| C5orf47 | 0.003673 |
| OR2L3 | 0.003672 |
| FCN2 | 0.003672 |
| BRD3 | 0.003671 |
| CDC5L | 0.003670 |
| HDAC1 | 0.003670 |
| CPLX4 | 0.003670 |
| MEPE | 0.003670 |
| SMARCD1 | 0.003669 |
| SDCCAG3 | 0.003668 |
| RBBP6 | 0.003667 |
| SCLY | 0.003666 |
| ZNF512 | 0.003665 |
| INSL5 | 0.003665 |
| NLE1 | 0.003665 |
| SIN3B | 0.003664 |
| ZNF407 | 0.003664 |
| ZSCAN22 | 0.003660 |
| STON2 | 0.003660 |
| DDI1 | 0.003659 |
| ZNF160 | 0.003658 |
| FBXO22 | 0.003658 |
| TM4SF20 | 0.003658 |
| TMEM67 | 0.003658 |
| C9orf50 | 0.003657 |
| AMER1 | 0.003656 |
| WIZ | 0.003656 |
| ASXL1 | 0.003656 |
| C18orf25 | 0.003655 |
| PGAM5 | 0.003655 |
| PRR23D2 | 0.003655 |
| ACD | 0.003654 |
| SETDB1 | 0.003653 |
| ZNF197 | 0.003653 |
| MAP3K4 | 0.003651 |
| RP1L1 | 0.003650 |
| TCEANC | 0.003648 |
| SLC28A2 | 0.003647 |
| PLEKIIG2 | 0.003647 |
| PPEF1 | 0.003646 |
| ABCD4 | 0.003646 |
| RFPL1 | 0.003645 |
| NARS2 | 0.003645 |
| MIF | 0.003644 |
| LSMEM2 | 0.003643 |
| MLXIPL | 0.003643 |
| ANXA2R | 0.003642 |
| RANBP6 | 0.003639 |
| PAN3 | 0.003639 |
| FAM131A | 0.003638 |
| CHTOP | 0.003638 |
| SERAC1 | 0.003638 |
| ATXN2L | 0.003637 |
| HBS1L | 0.003636 |
| UBXN2B | 0.003636 |
| WDR6 | 0.003634 |
| SRRM1 | 0.003633 |
| ADAT3 | 0.003633 |
| CAND2 | 0.003633 |
| PPAN | 0.003633 |
| NLRP13 | 0.003633 |
| NKRF | 0.003632 |
| BPIFB6 | 0.003632 |
| CRX | 0.003632 |
| FADS1 | 0.003631 |
| CSNK2A3 | 0.003630 |
| FOXK2 | 0.003630 |
| PSMG3 | 0.003629 |
| ZNF823 | 0.003628 |
| PTCD3 | 0.003627 |
| ATRX | 0.003626 |
| PSG4 | 0.003622 |
| KNCN | 0.003622 |
| RCE1 | 0.003621 |
| GTF2A1 | 0.003618 |
| GTF3C4 | 0.003618 |
| GALNT7 | 0.003616 |
| TYK2 | 0.003614 |
| SLC9A4 | 0.003614 |
| RAPGEFL1 | 0.003613 |
| MDGA2 | 0.003612 |
| EYS | 0.003612 |
| CBR4 | 0.003609 |
| GPHA2 | 0.003607 |
| TMEM79 | 0.003606 |
| PRSS55 | 0.003605 |
| SLC1A2 | 0.003600 |
| HESX1 | 0.003600 |
| C20orf96 | 0.003600 |
| USPL1 | 0.003599 |
| WDR54 | 0.003598 |
| RUVBL1 | 0.003597 |
| ZSCAN20 | 0.003597 |
| SMG5 | 0.003597 |
| COL10A1 | 0.003596 |
| LRRC61 | 0.003594 |
| UNC119 | 0.003591 |
| DRD5 | 0.003590 |
| DIDO1 | 0.003590 |
| KANSL1 | 0.003589 |
| STK11IP | 0.003588 |
| WDR19 | 0.003588 |
| GOLGA6L2 | 0.003586 |
| HS2ST1 | 0.003585 |
| OPN4 | 0.003583 |
| MARS | 0.003579 |
| TRMT2A | 0.003579 |
| NANP | 0.003578 |
| PRH1 | 0.003576 |
| ZNF223 | 0.003574 |
| PHF21A | 0.003566 |
| TRMT6 | 0.003565 |
| ZNF839 | 0.003564 |
| KRTAP10-10 | 0.003563 |
| GID8 | 0.003562 |
| OR52I1 | 0.003562 |
| LRRTM3 | 0.003560 |
| HNRNPA1L2 | 0.003560 |
| ZNF317 | 0.003559 |
| ANKIB1 | 0.003558 |
| FNDC8 | 0.003558 |
| BCLAF1 | 0.003557 |
| VANGL1 | 0.003557 |
| KHDRBS3 | 0.003557 |
| SLC30A3 | 0.003556 |
| INO80D | 0.003555 |
| NOL11 | 0.003555 |
| POLR2J3 | 0.003555 |
| PRRC2A | 0.003552 |
| LIN52 | 0.003552 |
| RCAN3 | 0.003549 |
| POLH | 0.003548 |
| TNRC6C | 0.003546 |
| ALKBH2 | 0.003546 |
| U2AF1 | 0.003546 |
| DNAJC10 | 0.003545 |
| RBM45 | 0.003544 |
| SUPT20H | 0.003544 |
| OSGEPL1 | 0.003544 |
| IFNK | 0.003541 |
| PTBP1 | 0.003541 |
| NAP1L4 | 0.003541 |
| CNTD1 | 0.003540 |
| RBM46 | 0.003539 |
| KLHL41 | 0.003537 |
| CNGB1 | 0.003537 |
| NSUN2 | 0.003537 |
| YDJC | 0.003537 |
| ZNF436 | 0.003535 |
| MAML3 | 0.003533 |
| GLCCH | 0.003531 |
| LRCH3 | 0.003530 |
| ZNF580 | 0.003529 |
| ZNF324B | 0.003527 |
| ANP32E | 0.003527 |
| ANKRD52 | 0.003527 |
| NANOS2 | 0.003525 |
| KRTAP10-6 | 0.003524 |
| SLC36A1 | 0.003523 |
| NUP160 | 0.003521 |
| PTP4A3 | 0.003519 |
| TUBGCP5 | 0.003519 |
| DENR | 0.003519 |
| NUDT21 | 0.003518 |
| RANGAP1 | 0.003517 |
| RABGGTB | 0.003516 |
| MSTO1 | 0.003515 |
| TAS2R42 | 0.003515 |
| LGALS14 | 0.003515 |
| CYFIP2 | 0.003514 |
| CCT5 | 0.003513 |
| XYLT1 | 0.003513 |
| LOXL3 | 0.003513 |
| FAM210A | 0.003512 |
| ORC4 | 0.003510 |
| LDLRAD4 | 0.003510 |
| GNG13 | 0.003507 |
| RFX7 | 0.003507 |
| NUMA1 | 0.003507 |
| ZNF222 | 0.003507 |
| CASP3 | 0.003506 |
| TMEM177 | 0.003504 |
| ATP11C | 0.003504 |
| ZNF770 | 0.003503 |
| DHX33 | 0.003501 |
| GABRA1 | 0.003501 |
| CHERP | 0.003501 |
| HHLA1 | 0.003500 |
| ACSBG2 | 0.003500 |
| PER3 | 0.003500 |
| OR52R1 | 0.003500 |
| PDZRN4 | 0.003500 |
| RIMS3 | 0.003499 |
| ZNF778 | 0.003499 |
| RGS12 | 0.003499 |
| KCTD1 | 0.003498 |
| DEFB134 | 0.003498 |
| CBWD2 | 0.003496 |
| DTNB | 0.003496 |
| FBXL18 | 0.003496 |
| ZBTB40 | 0.003496 |
| SNRPF | 0.003496 |
| RAB15 | 0.003496 |
| PGBD4 | 0.003496 |
| ZNF335 | 0.003493 |
| RPAP3 | 0.003492 |
| UBA5 | 0.003491 |
| TFF1 | 0.003490 |
| CCDC166 | 0.003489 |
| RMND5A | 0.003488 |
| ZBTB11 | 0.003487 |
| RNF112 | 0.003486 |
| LAS1L | 0.003486 |
| MEN1 | 0.003486 |
| USP28 | 0.003485 |
| TBCD | 0.003485 |
| MYPOP | 0.003484 |
| ZNF140 | 0.003484 |
| PCF11 | 0.003484 |
| GRM6 | 0.003484 |
| PHF12 | 0.003484 |
| OR2AK2 | 0.003484 |
| YAF2 | 0.003482 |
| XCL1 | 0.003482 |
| CCDC144A | 0.003481 |
| CASD1 | 0.003478 |
| CATSPER3 | 0.003478 |
| OR51S1 | 0.003477 |
| SLC5A6 | 0.003475 |
| GOLGA6L6 | 0.003475 |
| LRRC42 | 0.003475 |
| HEATR6 | 0.003475 |
| SLC45A1 | 0.003475 |
| XK | 0.003474 |
| MED20 | 0.003474 |
| ARL13A | 0.003473 |
| COPS5 | 0.003473 |
| PHOX2B | 0.003472 |
| CCNT1 | 0.003472 |
| ZNF471 | 0.003469 |
| ZBTB48 | 0.003469 |
| RBMXL2 | 0.003467 |
| WDR70 | 0.003467 |
| CNOT6 | 0.003466 |
| FAM83C | 0.003466 |
| REGIA | 0.003465 |
| CAM | 0.003465 |
| IMPA1 | 0.003464 |
| TAF1C | 0.003464 |
| P2RX5 | 0.003463 |
| NSUN5 | 0.003460 |
| DHX9 | 0.003459 |
| ZFPM1 | 0.003459 |
| ZNF275 | 0.003456 |
| RBM5 | 0.003456 |
| ELK1 | 0.003455 |
| ANKRD62 | 0.003455 |
| STAG2 | 0.003453 |
| TROVE2 | 0.003452 |
| C15orf61 | 0.003451 |
| FBXL8 | 0.003450 |
| ORC5 | 0.003450 |
| MROH1 | 0.003449 |
| PANK2 | 0.003448 |
| ZAN | 0.003448 |
| TET2 | 0.003447 |
| ZSCAN18 | 0.003446 |
| CLDN14 | 0.003446 |
| B4GALT2 | 0.003445 |
| ZNF586 | 0.003445 |
| SPIN1 | 0.003444 |
| HEATR9 | 0.003444 |
| GAGE2A | 0.003442 |
| MAP3K7 | 0.003440 |
| MBOAT4 | 0.003438 |
| C1orf105 | 0.003437 |
| FKBP6 | 0.003436 |
| CLMN | 0.003436 |
| PRPF38B | 0.003436 |
| PHLPP1 | 0.003435 |
| RPL39L | 0.003434 |
| LY6G6C | 0.003433 |
| E2F6 | 0.003432 |
| LATS1 | 0.003430 |
| PGLYRP2 | 0.003429 |
| DOLPP1 | 0.003428 |
| UMPS | 0.003427 |
| DNAJA4 | 0.003426 |
| ERCC2 | 0.003426 |
| ZNF598 | 0.003424 |
| FAM217B | 0.003423 |
| PHF13 | 0.003421 |
| ACTN3 | 0.003421 |
| ICOSLG | 0.003420 |
| OXGR1 | 0.003420 |
| LEKR1 | 0.003419 |
| DGKH | 0.003418 |
| PRIMPOL | 0.003418 |
| KCNA7 | 0.003417 |
| TBP | 0.003416 |
| NOL12 | 0.003416 |
| GALNTL5 | 0.003416 |
| RHNO1 | 0.003416 |
| PRSS3 | 0.003416 |
| NCBP2 | 0.003415 |
| NAA30 | 0.003414 |
| HIP1R | 0.003413 |
| SMYD1 | 0.003412 |
| MUCL1 | 0.003410 |
| ADAL | 0.003409 |
| STPG2 | 0.003409 |
| SLC25A41 | 0.003408 |
| YPEL4 | 0.003408 |
| BBIP1 | 0.003408 |
| A1BG | 0.003407 |
| USP21 | 0.003407 |
| OR51A7 | 0.003407 |
| TSC1 | 0.003406 |
| SGPL1 | 0.003405 |
| VHLL | 0.003404 |
| RPA3 | 0.003404 |
| PARD6A | 0.003401 |
| DEPDC5 | 0.003401 |
| MYOG | 0.003400 |
| RHBDD3 | 0.003400 |
| NGFR | 0.003399 |
| NR2C2 | 0.003398 |
| WNT6 | 0.003398 |
| NUDT10 | 0.003398 |
| KIAA1107 | 0.003396 |
| AURKC | 0.003396 |
| SLC17A2 | 0.003396 |
| LBHD1 | 0.003396 |
| CCK | 0.003395 |
| COA7 | 0.003395 |
| KANSL2 | 0.003395 |
| LAMB4 | 0.003395 |
| SH3BP5L | 0.003394 |
| RPS6KC1 | 0.003393 |
| ZNF501 | 0.003392 |
| HSP90AA1 | 0.003392 |
| KRTAP10-7 | 0.003391 |
| SMC5 | 0.003391 |
| ZNF844 | 0.003391 |
| NFATC4 | 0.003389 |
| PLEKHH3 | 0.003388 |
| NSUN7 | 0.003388 |
| ITIH4 | 0.003388 |
| POLR1C | 0.003387 |
| LRP11 | 0.003387 |
| BECN2 | 0.003384 |
| BRMS1L | 0.003383 |
| MLLT10 | 0.003383 |
| ZNF155 | 0.003383 |
| MAP9 | 0.003382 |
| REG4 | 0.003381 |
| C11orf65 | 0.003381 |
| GOLGA8J | 0.003380 |
| FAM69B | 0.003380 |
| SPATA9 | 0.003379 |
| ZBTB33 | 0.003377 |
| TTC31 | 0.003376 |
| GABPB1 | 0.003374 |
| ZNF136 | 0.003374 |
| RABL6 | 0.003374 |
| ZNF707 | 0.003374 |
| GTF3C3 | 0.003371 |
| CFHR2 | 0.003371 |
| MRPL30 | 0.003371 |
| CFAP70 | 0.003370 |
| KRBOX4 | 0.003369 |
| UQCRH | 0.003369 |
| OR10G2 | 0.003368 |
| EFCAB2 | 0.003368 |
| MAP2K7 | 0.003366 |
| PADI6 | 0.003366 |
| YWHAQ | 0.003365 |
| SIRT7 | 0.003365 |
| GLTSCR1 | 0.003364 |
| PRR20C | 0.003364 |
| SIAH2 | 0.003364 |
| EP400 | 0.003362 |
| CHN1 | 0.003362 |
| DNAJC2 | 0.003361 |
| ZNF354A | 0.003360 |
| PRR25 | 0.003360 |
| PBRM1 | 0.003359 |
| C8orf59 | 0.003357 |
| ITGA7 | 0.003357 |
| KRT40 | 0.003357 |
| MAB21L2 | 0.003356 |
| CCDC88A | 0.003355 |
| DENND1A | 0.003354 |
| WNT10B | 0.003354 |
| RABL2A | 0.003352 |
| STARD9 | 0.003351 |
| LYSMD4 | 0.003350 |
| ESM1 | 0.003350 |
| XRN1 | 0.003349 |
| PNPT1 | 0.003349 |
| CEP350 | 0.003348 |
| TTC39C | 0.003347 |
| SCRG1 | 0.003346 |
| MBD4 | 0.003346 |
| DHTKD1 | 0.003346 |
| NR2C1 | 0.003345 |
| INTS4 | 0.003343 |
| SEPHS1 | 0.003342 |
| RCN2 | 0.003342 |
| NOP58 | 0.003341 |
| ZNF212 | 0.003341 |
| GARNL3 | 0.003341 |
| MAGEC3 | 0.003340 |
| LRBA | 0.003339 |
| MTRF1L | 0.003338 |
| ELP2 | 0.003337 |
| C2orf69 | 0.003337 |
| CSPP1 | 0.003336 |
| VDAC3 | 0.003335 |
| VPS13B | 0.003334 |
| ORC3 | 0.003334 |
| COX20 | 0.003334 |
| HNRNPAB | 0.003334 |
| TNFSF11 | 0.003333 |
| GSTZ1 | 0.003329 |
| IRAK1BP1 | 0.003329 |
| TSPY10 | 0.003329 |
| KCNQ5 | 0.003329 |
| C1orf174 | 0.003328 |
| NACAD | 0.003327 |
| IQCH | 0.003327 |
| PGK2 | 0.003326 |
| C17orf58 | 0.003326 |
| STK11 | 0.003325 |
| NDUFAF4 | 0.003325 |
| MTRF1 | 0.003323 |
| TRIM66 | 0.003323 |
| TMPRSS11F | 0.003323 |
| PRRC2B | 0.003322 |
| PLD6 | 0.003321 |
| FARSA | 0.003321 |
| TMPRSS11D | 0.003320 |
| NCOA5 | 0.003319 |
| KRTAP10-4 | 0.003318 |
| TTBK2 | 0.003317 |
| RRH | 0.003313 |
| AGPAT4 | 0.003313 |
| LOXL2 | 0.003312 |
| SUPT7L | 0.003312 |
| ZKSCAN5 | 0.003312 |
| MBD3 | 0.003312 |
| WDPCP | 0.003311 |
| GNB5 | 0.003311 |
| SSRP1 | 0.003310 |
| PANKA | 0.003307 |
| KATNAL1 | 0.003305 |
| AVPR1A | 0.003305 |
| MTG2 | 0.003305 |
| SUB1 | 0.003304 |
| ZPLD1 | 0.003304 |
| LRRC20 | 0.003304 |
| TUBA3C | 0.003303 |
| NEURL2 | 0.003301 |
| FAM19A5 | 0.003301 |
| PTCD1 | 0.003301 |
| WSB2 | 0.003300 |
| ING3 | 0.003300 |
| GSK3B | 0.003299 |
| NOM1 | 0.003295 |
| COQ6 | 0.003294 |
| SMAD4 | 0.003293 |
| HMBOX1 | 0.003293 |
| EXOSC5 | 0.003291 |
| SIVA1 | 0.003291 |
| ARR3 | 0.003291 |
| PPIAL4G | 0.003290 |
| ACSM6 | 0.003288 |
| ACMSD | 0.003288 |
| OPALIN | 0.003288 |
| DUSP12 | 0.003287 |
| USP18 | 0.003285 |
| NPS | 0.003285 |
| DDX49 | 0.003284 |
| NEMP2 | 0.003283 |
| OR56A5 | 0.003283 |
| ZFR | 0.003282 |
| IARS | 0.003282 |
| NKAIN3 | 0.003282 |
| ATG2B | 0.003281 |
| MIER3 | 0.003280 |
| PPM1A | 0.003280 |
| CSPG5 | 0.003279 |
| GON4L | 0.003279 |
| FAM134B | 0.003277 |
| HNRNPU | 0.003276 |
| MON2 | 0.003275 |
| ACTR5 | 0.003275 |
| CD3EAP | 0.003274 |
| MARS2 | 0.003273 |
| RBBP5 | 0.003271 |
| ZMYM4 | 0.003271 |
| MTNR1A | 0.003270 |
| SLC2A5 | 0.003269 |
| ZNF263 | 0.003269 |
| GNB1L | 0.003269 |
| CGB1 | 0.003268 |
| SAE1 | 0.003267 |
| CLPB | 0.003267 |
| JMY | 0.003266 |
| HEATR4 | 0.003265 |
| DICER1 | 0.003265 |
| RD3 | 0.003264 |
| ZNF70 | 0.003263 |
| ZNF740 | 0.003262 |
| IFT27 | 0.003262 |
| ASB1 | 0.003262 |
| LANCL1 | 0.003261 |
| ERCC6 | 0.003260 |
| TXNL4B | 0.003259 |
| CYHR1 | 0.003258 |
| CDK20 | 0.003258 |
| RINT1 | 0.003257 |
| DNAJC28 | 0.003257 |
| DLG1 | 0.003257 |
| RRP1 | 0.003255 |
| HOMER2 | 0.003255 |
| ZSCAN29 | 0.003255 |
| C1orf52 | 0.003254 |
| ADGRG7 | 0.003254 |
| EIF4G3 | 0.003254 |
| SOHLH2 | 0.003254 |
| AMPD3 | 0.003254 |
| AKR1D1 | 0.003254 |
| IGHMBP2 | 0.003253 |
| PDSS1 | 0.003253 |
| COX6C | 0.003250 |
| TMEM69 | 0.003249 |
| GATC | 0.003249 |
| POMT1 | 0.003249 |
| GOLGA6L1 | 0.003248 |
| PRPF38A | 0.003247 |
| NUFIP2 | 0.003247 |
| ZC3H11A | 0.003244 |
| PPIP5K2 | 0.003243 |
| GTPBP8 | 0.003243 |
| FAM104A | 0.003243 |
| NOL4L | 0.003242 |
| ER13 | 0.003241 |
| SPEN | 0.003240 |
| ANKH | 0.003239 |
| NUDCD1 | 0.003239 |
| TAS2R39 | 0.003239 |
| STX2 | 0.003238 |
| KIIDRBS1 | 0.003238 |
| OR56A3 | 0.003238 |
| CCDC157 | 0.003237 |
| GET4 | 0.003237 |
| LMBR1 | 0.003236 |
| RBM24 | 0.003233 |
| AZIN1 | 0.003232 |
| RLIM | 0.003232 |
| SLC2A11 | 0.003231 |
| FOLH1 | 0.003231 |
| SSC4D | 0.003231 |
| SMG7 | 0.003230 |
| BRAF | 0.003229 |
| ZNF791 | 0.003229 |
| SLC37A4 | 0.003229 |
| GOPC | 0.003227 |
| PLCZ1 | 0.003227 |
| ZNF398 | 0.003226 |
| ZNF618 | 0.003225 |
| FAM98C | 0.003224 |
| PIAS3 | 0.003224 |
| FAM173A | 0.003221 |
| LMNTD2 | 0.003221 |
| CLCNKA | 0.003220 |
| KHSRP | 0.003219 |
| ZNF287 | 0.003219 |
| PRPSAPJ | 0.003218 |
| ICE2 | 0.003217 |
| SPSB3 | 0.003216 |
| STXBP4 | 0.003216 |
| USPI7L4 | 0.003214 |
| CPSF4L | 0.003214 |
| ZFP3 | 0.003213 |
| SLC47A2 | 0.003212 |
| OR5IJI | 0.003212 |
| C7orf34 | 0.003211 |
| STK35 | 0.003210 |
| CCT2 | 0.003210 |
| CNOT7 | 0.003209 |
| RFESD | 0.003207 |
| CUL4B | 0.003207 |
| SERPINE3 | 0.003207 |
| IL20RB | 0.003206 |
| DDX17 | 0.003203 |
| RPE | 0.003202 |
| NSD1 | 0.003198 |
| SSX2B | 0.003197 |
| RICTOR | 0.003197 |
| MPLKIP | 0.003197 |
| WDR31 | 0.003196 |
| CDK2AP1 | 0.003196 |
| IISFY1 | 0.003195 |
| KRTAP10-2 | 0.003195 |
| POLD2 | 0.003194 |
| ABIID11 | 0.003193 |
| IIEY2 | 0.003191 |
| MEAF6 | 0.003189 |
| KBTBD12 | 0.003188 |
| SPRR1A | 0.003188 |
| NROB2 | 0.003187 |
| PIKFYVE | 0.003187 |
| WNT8A | 0.003186 |
| ATF2 | 0.003185 |
| METTL21A | 0.003185 |
| PMS2 | 0.003184 |
| RBM8A | 0.003184 |
| ZNF649 | 0.003183 |
| SBF2 | 0.003182 |
| TCFL5 | 0.003182 |
| ZAR1L | 0.003182 |
| AFG3L2 | 0.003182 |
| RND2 | 0.003181 |
| 43525 | 0.003181 |
| SRPK2 | 0.003179 |
| CCDC27 | 0.003178 |
| GATAD2B | 0.003178 |
| C17orf67 | 0.003176 |
| CRY1 | 0.003176 |
| CHDH | 0.003174 |
| METTL10 | 0.003174 |
| TFAP2D | 0.003173 |
| NDRG4 | 0.003173 |
| MLNR | 0.003173 |
| ZNF280D | 0.003172 |
| TRIM55 | 0.003171 |
| KLHDC3 | 0.003170 |
| ARIH2OS | 0.003169 |
| CERS4 | 0.003167 |
| INPP5F | 0.003164 |
| ASPSCR1 | 0.003162 |
| RBMXL3 | 0.003160 |
| CCDC58 | 0.003160 |
| C6orf10 | 0.003160 |
| MED27 | 0.003160 |
| KCND1 | 0.003159 |
| AP5M1 | 0.003159 |
| ZNF134 | 0.003159 |
| HSD3B2 | 0.003158 |
| DLG4 | 0.003158 |
| FIZ1 | 0.003158 |
| CDON | 0.003156 |
| B3GNTL1 | 0.003155 |
| PFDN4 | 0.003155 |
| ZC3H6 | 0.003155 |
| PDCD6 | 0.003155 |
| GNL3 | 0.003153 |
| USP26 | 0.003152 |
| OR13D1 | 0.003151 |
| DDX56 | 0.003147 |
| OR1J4 | 0.003147 |
| ZNF595 | 0.003146 |
| DZIP1 | 0.003146 |
| GNG2 | 0.003144 |
| BBS7 | 0.003144 |
| C1orf204 | 0.003143 |
| ZBTB44 | 0.003143 |
| ALK | 0.003143 |
| LRPPRC | 0.003143 |
| VTA1 | 0.003143 |
| URB1 | 0.003143 |
| RNF126 | 0.003142 |
| MTX2 | 0.003141 |
| THAP5 | 0.003141 |
| NEFH | 0.003140 |
| KDM7A | 0.003137 |
| GMEB2 | 0.003135 |
| MRPS30 | 0.003135 |
| PDZD3 | 0.003134 |
| CPEB3 | 0.003134 |
| PSG1 | 0.003134 |
| KRTAP19-1 | 0.003133 |
| SLC9A5 | 0.003132 |
| SZT2 | 0.003131 |
| CFAP61 | 0.003131 |
| TMEM97 | 0.003130 |
| NEU4 | 0.003130 |
| TMEM80 | 0.003130 |
| CNTFR | 0.003129 |
| PCM1 | 0.003129 |
| DCAKD | 0.003129 |
| SPATA7 | 0.003129 |
| ASF1A | 0.003128 |
| GFM2 | 0.003128 |
| CAPS2 | 0.003128 |
| CFAP54 | 0.003125 |
| POLB | 0.003125 |
| GSTCD | 0.003125 |
| PPM1D | 0.003123 |
| DSG1 | 0.003123 |
| USP14 | 0.003121 |
| TAS1R3 | 0.003121 |
| FYTTD1 | 0.003121 |
| NEMF | 0.003121 |
| CCT3 | 0.003121 |
| TMCO6 | 0.003120 |
| CCDC137 | 0.003120 |
| SHOX | 0.003119 |
| PLA2G2F | 0.003119 |
| SPATA5 | 0.003116 |
| SFMBT1 | 0.003116 |
| IGFL2 | 0.003116 |
| DHX57 | 0.003112 |
| ADGRL3 | 0.003111 |
| NBPF9 | 0.003111 |
| COPS7B | 0.003109 |
| FGL1 | 0.003105 |
| GPI | 0.003105 |
| ARMC6 | 0.003104 |
| FYN | 0.003104 |
| SCGB2A2 | 0.003104 |
| RPRD1B | 0.003104 |
| ALS2 | 0.003104 |
| P2RX6 | 0.003103 |
| TAS2R1 | 0.003101 |
| HID1 | 0.003100 |
| GATA4 | 0.003099 |
| SIRT6 | 0.003099 |
| ERH | 0.003098 |
| ZNF276 | 0.003097 |
| KDM6A | 0.003096 |
| FADS6 | 0.003096 |
| WDR86 | 0.003095 |
| THAP2 | 0.003094 |
| ARFGEF3 | 0.003094 |
| CPXM1 | 0.003094 |
| ENTPD4 | 0.003093 |
| CLASP1 | 0.003093 |
| GAPDH | 0.003093 |
| VPS16 | 0.003093 |
| DEAF1 | 0.003093 |
| DDX27 | 0.003092 |
| AAAS | 0.003091 |
| PEX6 | 0.003091 |
| OR13J1 | 0.003090 |
| ANGEL2 | 0.003089 |
| SYNJ1 | 0.003089 |
| OR2L5 | 0.003088 |
| GALNT14 | 0.003087 |
| PUSL1 | 0.003086 |
| SLC25A22 | 0.003084 |
| ANAPC4 | 0.003083 |
| INVS | 0.003082 |
| SSX1 | 0.003082 |
| ZCCHC7 | 0.003081 |
| ADNP2 | 0.003081 |
| MTMR9 | 0.003081 |
| SP7 | 0.003081 |
| CYCS | 0.003078 |
| EIF4A3 | 0.003076 |
| OR4F3 | 0.003075 |
| FBXO48 | 0.003075 |
| TPD52L3 | 0.003075 |
| C12orf40 | 0.003073 |
| TARSL2 | 0.003072 |
| ZNF12 | 0.003070 |
| BOD1L2 | 0.003070 |
| TTC5 | 0.003069 |
| TMEM55B | 0.003069 |
| ZNF587B | 0.003069 |
| CARMI | 0.003068 |
| MGEA5 | 0.003068 |
| CLCN5 | 0.003068 |
| TNRC6B | 0.003067 |
| SNRPB | 0.003066 |
| RIOK1 | 0.003065 |
| DCUN1D1 | 0.003063 |
| REC114 | 0.003059 |
| GUF1 | 0.003055 |
| UBTF | 0.003054 |
| GXYLT1 | 0.003053 |
| MYLK3 | 0.003053 |
| MTFR1 | 0.003051 |
| INHBB | 0.003051 |
| TNP2 | 0.003048 |
| EXD3 | 0.003047 |
| GTF2I | 0.003047 |
| SF3B1 | 0.003046 |
| MANEA | 0.003043 |
| PAWR | 0.003041 |
| TTC26 | 0.003041 |
| TAAR3 | 0.003041 |
| KRTAP10-11 | 0.003039 |
| DPY19L4 | 0.003038 |
| ZNF132 | 0.003038 |
| PI4KA | 0.003037 |
| ARMC8 | 0.003037 |
| TBRG4 | 0.003036 |
| FGF4 | 0.003036 |
| CEMP1 | 0.003036 |
| PDK3 | 0.003035 |
| RALGDS | 0.003035 |
| PRSS45 | 0.003035 |
| MYH7 | 0.003033 |
| SET | 0.003033 |
| PGBD2 | 0.003032 |
| SECISBP2 | 0.003032 |
| HEBP2 | 0.003031 |
| POLR2I | 0.003031 |
| RANBP9 | 0.003029 |
| KCNG2 | 0.003028 |
| BMF | 0.003028 |
| KRR1 | 0.003027 |
| ANKHD1 | 0.003027 |
| NAA50 | 0.003026 |
| ETV2 | 0.003024 |
| TOMM5 | 0.003024 |
| GABPB2 | 0.003023 |
| GALNTL6 | 0.003023 |
| ATP11B | 0.003022 |
| PTPN2 | 0.003020 |
| LGALS7 | 0.003020 |
| ZNF440 | 0.003018 |
| GPALPP1 | 0.003018 |
| ZNF773 | 0.003017 |
| 43527 | 0.003016 |
| KRTAP1-1 | 0.003016 |
| SLC39A3 | 0.003016 |
| WRNIP1 | 0.003015 |
| ESF1 | 0.003015 |
| C9orf43 | 0.003014 |
| FBXL4 | 0.003014 |
| ZNF621 | 0.003014 |
| RP9 | 0.003013 |
| FAM9B | 0.003010 |
| OR2H1 | 0.003006 |
| WDR73 | 0.003005 |
| TMEM209 | 0.003004 |
| ZW10 | 0.003004 |
| ACTR6 | 0.003003 |
| NCOA6 | 0.003003 |
| OR8B12 | 0.003002 |
| SLC35G5 | 0.003002 |
| FSBP | 0.003002 |
| TRAF2 | 0.003001 |
| KRTAP5-8 | 0.003001 |
| CDH20 | 0.003000 |
| TBCC | 0.002999 |
| TLK2 | 0.002998 |
| ZBTB24 | 0.002997 |
| SMARCC1 | 0.002996 |
| AKAP9 | 0.002993 |
| PFDN6 | 0.002993 |
| MEDIO | 0.002993 |
| CIPC | 0.002992 |
| FAM213A | 0.002992 |
| HELZ | 0.002991 |
| SGSM3 | 0.002991 |
| AMBN | 0.002990 |
| STK19 | 0.002990 |
| NFATC2IP | 0.002989 |
| MPDZ | 0.002988 |
| TMEM120B | 0.002987 |
| AGBL5 | 0.002986 |
| OR1K1 | 0.002986 |
| RBMX2 | 0.002986 |
| TEKT2 | 0.002983 |
| NR6A1 | 0.002983 |
| HNRNPC | 0.002981 |
| PET100 | 0.002981 |
| ARID3B | 0.002980 |
| ZNF142 | 0.002980 |
| RAD17 | 0.002979 |
| ANKRD12 | 0.002979 |
| SPAG8 | 0.002977 |
| KIAA2026 | 0.002977 |
| TBC1D24 | 0.002976 |
| FAM205C | 0.002975 |
| FAM13B | 0.002974 |
| P2RY11 | 0.002974 |
| UAP1L1 | 0.002973 |
| TMEM183A | 0.002973 |
| CDK5RAP1 | 0.002972 |
| KCNU1 | 0.002972 |
| MARK3 | 0.002971 |
| NUP133 | 0.002968 |
| C7orf61 | 0.002967 |
| GABRR3 | 0.002967 |
| ANAPC2 | 0.002965 |
| KIAA1024L | 0.002965 |
| ZNF470 | 0.002965 |
| OBP2B | 0.002964 |
| C19orf81 | 0.002964 |
| NXF1 | 0.002964 |
| MORC2 | 0.002964 |
| OSR2 | 0.002964 |
| PPIH | 0.002962 |
| HYI | 0.002962 |
| KRTDAP | 0.002960 |
| SAMD13 | 0.002959 |
| NPRL2 | 0.002958 |
| C8G | 0.002956 |
| CISD2 | 0.002956 |
| UFD1L | 0.002956 |
| GPR32 | 0.002956 |
| MORF4L2 | 0.002955 |
| HSD11B1L | 0.002951 |
| AREL1 | 0.002951 |
| LRIF1 | 0.002951 |
| PSG9 | 0.002950 |
| FABP1 | 0.002950 |
| KIAA1614 | 0.002950 |
| HMGB4 | 0.002949 |
| PSMC6 | 0.002949 |
| SPATA31D1 | 0.002947 |
| DCAF8L2 | 0.002947 |
| MIS12 | 0.002947 |
| ZNF845 | 0.002946 |
| FAM210B | 0.002946 |
| HES3 | 0.002945 |
| HNRNPL | 0.002944 |
| HEATR1 | 0.002943 |
| OR52L1 | 0.002943 |
| WRAP53 | 0.002939 |
| RAB11FIP3 | 0.002939 |
| ELMOD2 | 0.002939 |
| TSGA13 | 0.002938 |
| HUWE1 | 0.002938 |
| MPPED2 | 0.002938 |
| SCAF4 | 0.002937 |
| EBF4 | 0.002937 |
| UBFD1 | 0.002937 |
| INPP5J | 0.002936 |
| CARHSP1 | 0.002935 |
| CXorf23 | 0.002935 |
| HSPH1 | 0.002934 |
| SLC33A1 | 0.002934 |
| FIP1L1 | 0.002933 |
| DDX46 | 0.002933 |
| C8orf22 | 0.002933 |
| PACRGL | 0.002933 |
| GABRB1 | 0.002933 |
| MRPL47 | 0.002931 |
| PGM3 | 0.002931 |
| QRSL1 | 0.002930 |
| APEX1 | 0.002929 |
| PKN2 | 0.002929 |
| DAAM1 | 0.002928 |
| OR10A5 | 0.002928 |
| PREPL | 0.002927 |
| MFSD12 | 0.002927 |
| ZADH2 | 0.002926 |
| SDHAF1 | 0.002925 |
| TIMM23B | 0.002925 |
| DNAJC18 | 0.002924 |
| ZBTB17 | 0.002923 |
| PPP2R5D | 0.002922 |
| CCDC90B | 0.002920 |
| CD200 | 0.002920 |
| PRSS48 | 0.002918 |
| POLR2B | 0.002918 |
| NF1 | 0.002917 |
| TTF1 | 0.002917 |
| UGT2A3 | 0.002916 |
| CYSRT1 | 0.002915 |
| FSHB | 0.002915 |
| CYP27B1 | 0.002914 |
| SCIN | 0.002914 |
| PSG7 | 0.002914 |
| CYP2R1 | 0.002913 |
| CMAS | 0.002913 |
| SGTA | 0.002912 |
| SH3BP2 | 0.002911 |
| SP5 | 0.002910 |
| GTPBP10 | 0.002909 |
| CACNG3 | 0.002908 |
| MTIF2 | 0.002908 |
| TSPO2 | 0.002907 |
| KCNK13 | 0.002907 |
| KRTAP5-9 | 0.002906 |
| BAIAP3 | 0.002906 |
| PIK3C3 | 0.002905 |
| CDC34 | 0.002905 |
| UBAP2 | 0.002903 |
| FGF16 | 0.002903 |
| XRCC5 | 0.002902 |
| C19orf60 | 0.002902 |
| ZNF513 | 0.002901 |
| EPC2 | 0.002899 |
| OR2AE1 | 0.002898 |
| FAM200A | 0.002893 |
| SULF1 | 0.002892 |
| PARS2 | 0.002890 |
| SCAF8 | 0.002890 |
| ZBED6 | 0.002890 |
| TRIM49D2 | 0.002890 |
| GNAT3 | 0.002889 |
| TIMM9 | 0.002888 |
| PROSER1 | 0.002888 |
| FGF23 | 0.002887 |
| TSC2 | 0.002887 |
| CRYBB3 | 0.002886 |
| CASK | 0.002886 |
| ARID4B | 0.002885 |
| CEACAM16 | 0.002885 |
| HS3ST1 | 0.002885 |
| CARD14 | 0.002883 |
| NAA10 | 0.002882 |
| NIPSNAP1 | 0.002881 |
| MCPH1 | 0.002880 |
| ADIPOQ | 0.002880 |
| OR8D1 | 0.002879 |
| TBCE | 0.002878 |
| TFF2 | 0.002877 |
| GMCL1 | 0.002877 |
| ATP9A | 0.002875 |
| PLEKHB1 | 0.002874 |
| FAM231D | 0.002874 |
| DTD1 | 0.002873 |
| CDK4 | 0.002872 |
| CSTF2 | 0.002871 |
| INTS1 | 0.002871 |
| PBX3 | 0.002870 |
| SGIP1 | 0.002870 |
| OR2L2 | 0.002870 |
| GCSH | 0.002870 |
| SRRM2 | 0.002869 |
| LYPD8 | 0.002869 |
| ZNF516 | 0.002869 |
| GZF1 | 0.002868 |
| STK33 | 0.002867 |
| LEMD3 | 0.002867 |
| POLR3E | 0.002867 |
| RAPGEF4 | 0.002866 |
| SLC25A39 | 0.002866 |
| POP7 | 0.002865 |
| ACAD9 | 0.002865 |
| GNPDA2 | 0.002865 |
| CDH10 | 0.002864 |
| EBLN1 | 0.002864 |
| KIT | 0.002863 |
| ZBTB21 | 0.002862 |
| OR13H1 | 0.002860 |
| RDH12 | 0.002859 |
| DAGLA | 0.002859 |
| AKAP8 | 0.002857 |
| TMEM170B | 0.002857 |
| SRM | 0.002857 |
| MRS2 | 0.002857 |
| CARS | 0.002855 |
| FKBPL | 0.002853 |
| GVQW1 | 0.002852 |
| NAPG | 0.002852 |
| SEC22A | 0.002852 |
| VAPB | 0.002851 |
| TLDC1 | 0.002851 |
| FSCB | 0.002850 |
| DUSP4 | 0.002850 |
| NIPA1 | 0.002849 |
| MASI | 0.002849 |
| USP30 | 0.002848 |
| NHLH2 | 0.002848 |
| DHX40 | 0.002848 |
| OR51QJ | 0.002847 |
| ZNF175 | 0.002847 |
| CXorf58 | 0.002845 |
| MTERF4 | 0.002844 |
| OVGP1 | 0.002843 |
| ZNF296 | 0.002842 |
| POLM | 0.002841 |
| ING4 | 0.002841 |
| MAPK6 | 0.002840 |
| CLNS1A | 0.002839 |
| TFAM | 0.002838 |
| ABCA10 | 0.002836 |
| GTF2H2C | 0.002836 |
| YLPM1 | 0.002836 |
| DNMT3L | 0.002835 |
| ASCC3 | 0.002835 |
| STEAP1B | 0.002835 |
| PSG11 | 0.002835 |
| IL17F | 0.002835 |
| DCAF12 | 0.002833 |
| CHD8 | 0.002832 |
| NFKBIL1 | 0.002832 |
| PHF2 | 0.002831 |
| NOP2 | 0.002831 |
| PRMT7 | 0.002831 |
| TMEM182 | 0.002830 |
| GIPR | 0.002830 |
| CCDC78 | 0.002830 |
| ZRANB1 | 0.002830 |
| SAYSD1 | 0.002827 |
| OR2C3 | 0.002826 |
| AMPD2 | 0.002826 |
| B4GALT4 | 0.002825 |
| RHOXF2B | 0.002825 |
| PPHLN1 | 0.002824 |
| CSNK2A1 | 0.002824 |
| RWDD3 | 0.002824 |
| NUP50 | 0.002823 |
| ZNF329 | 0.002823 |
| FRMD3 | 0.002822 |
| DBX2 | 0.002821 |
| OR2D2 | 0.002821 |
| CHFR | 0.002819 |
| TMEM107 | 0.002819 |
| ZNF28 | 0.002819 |
| WDR18 | 0.002816 |
| RAN | 0.002816 |
| GAGE13 | 0.002815 |
| LDHAL6B | 0.002815 |
| COX17 | 0.002814 |
| ZNF34 | 0.002814 |
| EPG5 | 0.002814 |
| FAM221B | 0.002812 |
| OR1N2 | 0.002811 |
| MYH15 | 0.002811 |
| C19orf25 | 0.002811 |
| SPATA31A1 | 0.002811 |
| COMTD1 | 0.002809 |
| OR1L3 | 0.002809 |
| ZXDB | 0.002809 |
| CRYAA | 0.002807 |
| PDS5B | 0.002807 |
| DDX5 | 0.002805 |
| ANKRD53 | 0.002805 |
| TOMM40 | 0.002805 |
| TXLNA | 0.002802 |
| C2orf49 | 0.002801 |
| MEMO1 | 0.002801 |
| MTAP | 0.002799 |
| TSHZ1 | 0.002799 |
| LRTM1 | 0.002799 |
| RPS6KB1 | 0.002796 |
| OR8G1 | 0.002795 |
| SSBP4 | 0.002794 |
| ZNF350 | 0.002794 |
| PAGE4 | 0.002793 |
| ARHGAP19 | 0.002790 |
| RHAG | 0.002790 |
| ATP1OB | 0.002790 |
| TRA2A | 0.002788 |
| HUS1 | 0.002788 |
| SLC37A3 | 0.002788 |
| LHB | 0.002787 |
| GGT7 | 0.002787 |
| ATF7IP | 0.002787 |
| BCAN | 0.002787 |
| NCKIPSD | 0.002787 |
| ZFP41 | 0.002787 |
| LEFTY1 | 0.002786 |
| PRPF40A | 0.002785 |
| TM4SF5 | 0.002785 |
| XKR4 | 0.002784 |
| TMEM14A | 0.002783 |
| ZNF33B | 0.002783 |
| SMIM21 | 0.002782 |
| RRP1B | 0.002782 |
| ZSCAN10 | 0.002782 |
| ZNF304 | 0.002780 |
| GRM5 | 0.002780 |
| CPOX | 0.002780 |
| CECR2 | 0.002777 |
| ZFP28 | 0.002774 |
| NLGN3 | 0.002774 |
| AKR1C4 | 0.002773 |
| SPP2 | 0.002772 |
| WTAP | 0.002772 |
| FAMS3A | 0.002772 |
| FAM227B | 0.002771 |
| FAM9C | 0.002771 |
| VN1R5 | 0.002770 |
| PPP2R1B | 0.002770 |
| LZIC | 0.002770 |
| ADAM28 | 0.002769 |
| SIAH1 | 0.002769 |
| CYP21A2 | 0.002769 |
| PSG8 | 0.002768 |
| EDC4 | 0.002768 |
| PTMA | 0.002767 |
| UTP18 | 0.002766 |
| GPATCH8 | 0.002765 |
| TAS2R9 | 0.002763 |
| MRPL48 | 0.002762 |
| CCDC180 | 0.002762 |
| RBM48 | 0.002761 |
| RBMY1A1 | 0.002760 |
| SNRNP40 | 0.002760 |
| ALG9 | 0.002759 |
| LONP1 | 0.002756 |
| DDX52 | 0.002756 |
| UTP20 | 0.002756 |
| MTA2 | 0.002755 |
| C1orf216 | 0.002754 |
| GYPA | 0.002754 |
| USP24 | 0.002754 |
| DCUN1D5 | 0.002753 |
| SPRTN | 0.002753 |
| DBR1 | 0.002752 |
| RBM19 | 0.002752 |
| SARDH | 0.002751 |
| KLC3 | 0.002751 |
| TSPY8 | 0.002749 |
| TSN | 0.002749 |
| FLAD1 | 0.002748 |
| TOE1 | 0.002748 |
| MAP4K5 | 0.002747 |
| SMARCC2 | 0.002747 |
| DLGAP2 | 0.002747 |
| POLR3K | 0.002746 |
| BCL11A | 0.002745 |
| PARG | 0.002744 |
| GPR21 | 0.002744 |
| ABHD16B | 0.002744 |
| EWSR1 | 0.002744 |
| FLG2 | 0.002744 |
| C8orf89 | 0.002743 |
| SPATA6L | 0.002743 |
| OPRM1 | 0.002742 |
| SLC9B1 | 0.002742 |
| PTGR2 | 0.002742 |
| MAML1 | 0.002742 |
| BRPF3 | 0.002741 |
| GNPNAT1 | 0.002740 |
| PICK1 | 0.002738 |
| CCT8 | 0.002738 |
| SPTA1 | 0.002737 |
| LSM2 | 0.002737 |
| GABRA6 | 0.002736 |
| ARG1 | 0.002736 |
| FBL | 0.002735 |
| LLGL1 | 0.002734 |
| C14orf2 | 0.002733 |
| MCM9 | 0.002733 |
| ZBTB14 | 0.002732 |
| SNRPD2 | 0.002732 |
| KRT74 | 0.002732 |
| PSG5 | 0.002732 |
| PTGES2 | 0.002730 |
| MBOAT2 | 0.002730 |
| FAM26D | 0.002729 |
| 43716 | 0.002728 |
| FAM133A | 0.002728 |
| LPIN3 | 0.002728 |
| TRPM2 | 0.002727 |
| FGF19 | 0.002727 |
| FAAH2 | 0.002727 |
| BMI1 | 0.002727 |
| FAT2 | 0.002726 |
| IFT74 | 0.002725 |
| ENY2 | 0.002725 |
| OR2AG2 | 0.002724 |
| TAS2R40 | 0.002724 |
| MED28 | 0.002723 |
| BTBD8 | 0.002723 |
| ARHGAP4 | 0.002723 |
| STRADA | 0.002723 |
| SERPINA6 | 0.002722 |
| CAPN15 | 0.002722 |
| CLDN22 | 0.002721 |
| FAM188A | 0.002719 |
| HNRNPM | 0.002719 |
| RNASE9 | 0.002719 |
| OR6A2 | 0.002719 |
| ZMYM5 | 0.002717 |
| TEX35 | 0.002717 |
| EVL | 0.002716 |
| OR5C1 | 0.002715 |
| CIDEB | 0.002715 |
| NUMBL | 0.002713 |
| TCHHL1 | 0.002713 |
| FAM193A | 0.002713 |
| PRKRA | 0.002712 |
| SCAMP1 | 0.002712 |
| FBXL22 | 0.002711 |
| CBWD1 | 0.002711 |
| SIRT5 | 0.002711 |
| CHKB | 0.002711 |
| DCDC1 | 0.002711 |
| OR9A4 | 0.002711 |
| SPRYD4 | 0.002710 |
| OR2D3 | 0.002709 |
| 43533 | 0.002709 |
| ANKRA2 | 0.002708 |
| HSPA13 | 0.002708 |
| USP34 | 0.002708 |
| SMARCB1 | 0.002707 |
| TECRL | 0.002706 |
| NRIP2 | 0.002706 |
| PLXNA4 | 0.002706 |
| NHLRC2 | 0.002705 |
| ACAD8 | 0.002705 |
| SPATA17 | 0.002704 |
| NKX2-6 | 0.002703 |
| ENTPD6 | 0.002702 |
| LARS | 0.002702 |
| CSTL1 | 0.002701 |
| C18orf32 | 0.002700 |
| SSBP2 | 0.002700 |
| FAM209A | 0.002700 |
| TCF20 | 0.002700 |
| GAGE2E | 0.002699 |
| ARID1B | 0.002699 |
| KIAA1257 | 0.002699 |
| ZNF511 | 0.002698 |
| CLK3 | 0.002698 |
| C19orf68 | 0.002698 |
| TTLL4 | 0.002698 |
| FAM160A2 | 0.002697 |
| ZNF500 | 0.002696 |
| FAM199X | 0.002696 |
| ZNF426 | 0.002695 |
| CMTR1 | 0.002693 |
| RANBP3 | 0.002693 |
| PRPS1 | 0.002692 |
| TRIM11 | 0.002692 |
| ZNF41 | 0.002691 |
| MBOAT1 | 0.002690 |
| RNF187 | 0.002690 |
| SPATC1L | 0.002690 |
| FBXO42 | 0.002689 |
| FCHSD1 | 0.002689 |
| ASAP2 | 0.002687 |
| KATNBL1 | 0.002686 |
| CERK | 0.002685 |
| PDZRN3 | 0.002685 |
| MKKS | 0.002683 |
| RTN3 | 0.002682 |
| EPCAM | 0.002680 |
| SFXN4 | 0.002680 |
| NUP54 | 0.002679 |
| TAAR8 | 0.002678 |
| TMEM52 | 0.002677 |
| ETAA1 | 0.002677 |
| ITSN1 | 0.002676 |
| NGDN | 0.002675 |
| NYAP2 | 0.002674 |
| PAGE1 | 0.002674 |
| TNPO1 | 0.002673 |
| NAA15 | 0.002673 |
| C7orf43 | 0.002671 |
| FBXO44 | 0.002670 |
| TCOF1 | 0.002670 |
| SLC5A2 | 0.002670 |
| MOV10 | 0.002669 |
| UPK3A | 0.002668 |
| KIN | 0.002667 |
| FBXO9 | 0.002666 |
| POFUT2 | 0.002665 |
| MRPL3 | 0.002665 |
| OGG1 | 0.002664 |
| PRB1 | 0.002663 |
| ZNF564 | 0.002659 |
| ARMC2 | 0.002659 |
| RAI1 | 0.002659 |
| NRG4 | 0.002659 |
| PDCD2 | 0.002657 |
| UHRF1BP1L | 0.002656 |
| PAGE2 | 0.002655 |
| SWT1 | 0.002654 |
| PJA1 | 0.002654 |
| DPY30 | 0.002653 |
| FRYL | 0.002652 |
| NOC2L | 0.002652 |
| UCK1 | 0.002650 |
| GFER | 0.002649 |
| KLHL7 | 0.002649 |
| PRR20A | 0.002647 |
| PRR20B | 0.002647 |
| PRR20D | 0.002647 |
| PRR20E | 0.002647 |
| WDR89 | 0.002647 |
| ZNF2 | 0.002647 |
| FAM161B | 0.002646 |
| TSEN2 | 0.002645 |
| SOS1 | 0.002644 |
| SIPA1L2 | 0.002642 |
| NPBWR2 | 0.002641 |
| MAGEB6 | 0.002640 |
| NT5C3A | 0.002639 |
| IQCA1L | 0.002639 |
| SNTG1 | 0.002638 |
| AP1AR | 0.002638 |
| RANBP2 | 0.002638 |
| USP9X | 0.002636 |
| PTCH1 | 0.002636 |
| BRD2 | 0.002636 |
| CRIPT | 0.002636 |
| C18orf21 | 0.002636 |
| XRCC1 | 0.002636 |
| C1orf226 | 0.002635 |
| TRAP1 | 0.002635 |
| TMEM263 | 0.002635 |
| YTHDC1 | 0.002634 |
| MED30 | 0.002633 |
| IPOS | 0.002633 |
| PRPF19 | 0.002632 |
| FABP12 | 0.002632 |
| OR1L4 | 0.002632 |
| UNC119B | 0.002632 |
| H1FOO | 0.002631 |
| ECSIT | 0.002629 |
| TARBP2 | 0.002629 |
| CBLB | 0.002629 |
| PTRH2 | 0.002628 |
| GOLGA6C | 0.002628 |
| ACLY | 0.002628 |
| TR1M43 | 0.002628 |
| AOC1 | 0.002628 |
| RPL7L1 | 0.002627 |
| OR5IA4 | 0.002626 |
| CLASP2 | 0.002626 |
| ANKRD34A | 0.002625 |
| PRPSAP2 | 0.002625 |
| ANKAR | 0.002625 |
| NDUFB1 | 0.002624 |
| RPP30 | 0.002623 |
| AZIN2 | 0.002623 |
| CIIAC1 | 0.002623 |
| ARID1A | 0.002622 |
| ASMT | 0.002622 |
| ZNF282 | 0.002622 |
| RALGAPB | 0.002621 |
| ST7L | 0.002621 |
| OCIAD2 | 0.002620 |
| MED1 | 0.002619 |
| FBXO11 | 0.002619 |
| TTC8 | 0.002619 |
| MTMR1 | 0.002618 |
| COA5 | 0.002618 |
| ZNF837 | 0.002618 |
| NLRP8 | 0.002618 |
| HSDL1 | 0.002617 |
| DNAJA3 | 0.002617 |
| COL5A2 | 0.002616 |
| MSANTD3 | 0.002616 |
| RNF24 | 0.002615 |
| OR2M4 | 0.002613 |
| PRB4 | 0.002613 |
| COG1 | 0.002612 |
| ZNRF2 | 0.002612 |
| FUZ | 0.002612 |
| POGLUT1 | 0.002611 |
| UCHL5 | 0.002611 |
| B3GAT3 | 0.002610 |
| RNF220 | 0.002610 |
| ATP2C1 | 0.002610 |
| P4HTM | 0.002609 |
| EIF2B1 | 0.002608 |
| ZNF165 | 0.002607 |
| MAP4K3 | 0.002607 |
| METTL2B | 0.002607 |
| SPATA31D3 | 0.002606 |
| C1QTNF6 | 0.002605 |
| SLC2A7 | 0.002603 |
| OR4F21 | 0.002603 |
| OR2J3 | 0.002602 |
| ATXN7L3B | 0.002602 |
| DIP2C | 0.002602 |
| ZFHX3 | 0.002602 |
| OR52E6 | 0.002601 |
| RAVER1 | 0.002601 |
| EIF5A2 | 0.002601 |
| PIH1D1 | 0.002601 |
| NRF1 | 0.002600 |
| PRR35 | 0.002600 |
| SPHK2 | 0.002600 |
| TRMT61B | 0.002599 |
| RABIF | 0.002599 |
| TRMT10A | 0.002599 |
| TLE1 | 0.002599 |
| SLC25A17 | 0.002599 |
| PPFIA1 | 0.002598 |
| MAP4K4 | 0.002598 |
| CAMSAP3 | 0.002598 |
| ZNF274 | 0.002598 |
| RNF214 | 0.002596 |
| USP17L11 | 0.002596 |
| RNASEH1 | 0.002595 |
| TBC1D19 | 0.002595 |
| STATH | 0.002594 |
| WDR47 | 0.002593 |
| NTSC | 0.002593 |
| UIMC1 | 0.002592 |
| CASZ1 | 0.002591 |
| PDHX | 0.002590 |
| C12orf76 | 0.002589 |
| CATSPERG | 0.002589 |
| ACAD10 | 0.002589 |
| MEGF8 | 0.002588 |
| FAM231A | 0.002588 |
| NMUR2 | 0.002588 |
| TLK1 | 0.002586 |
| APOPT1 | 0.002584 |
| FNTA | 0.002584 |
| RNF8 | 0.002583 |
| C6orf48 | 0.002583 |
| DNLZ | 0.002582 |
| CPSF2 | 0.002582 |
| BRD1 | 0.002581 |
| MOGAT3 | 0.002579 |
| RRAGD | 0.002577 |
| STRIP1 | 0.002575 |
| TNFSF4 | 0.002575 |
| LDHC | 0.002574 |
| CLK4 | 0.002572 |
| ACP1 | 0.002572 |
| OR51D1 | 0.002571 |
| KDM5C | 0.002571 |
| TRIM42 | 0.002570 |
| RRN3 | 0.002570 |
| FRMPD4 | 0.002569 |
| SMN1 | 0.002568 |
| OR2A14 | 0.002567 |
| YTHDC2 | 0.002566 |
| CPSF3 | 0.002565 |
| THBS2 | 0.002562 |
| CLCN6 | 0.002558 |
| FMR1NB | 0.002558 |
| BAIAP2L1 | 0.002557 |
| SLC35B4 | 0.002556 |
| PPP2R2D | 0.002556 |
| UGT2B15 | 0.002556 |
| CYP11B1 | 0.002556 |
| TAB3 | 0.002556 |
| TH | 0.002555 |
| OR11H12 | 0.002555 |
| C1orf50 | 0.002554 |
| ZBTB3 | 0.002552 |
| ZNF79 | 0.002552 |
| DDX10 | 0.002552 |
| STRN3 | 0.002551 |
| ZNF735 | 0.002549 |
| ARMCX5 | 0.002549 |
| SLC37A1 | 0.002548 |
| SMAD5 | 0.002547 |
| UTP6 | 0.002547 |
| PCCB | 0.002546 |
| MRPL1 | 0.002546 |
| VMA21 | 0.002544 |
| PMPCB | 0.002543 |
| METTL5 | 0.002543 |
| SAMD4B | 0.002543 |
| PAIP1 | 0.002543 |
| SFRP1 | 0.002542 |
| SPG7 | 0.002541 |
| COX19 | 0.002541 |
| THAP6 | 0.002539 |
| ABCB7 | 0.002539 |
| CDCA7L | 0.002539 |
| TIPRL | 0.002537 |
| ZMAT1 | 0.002536 |
| ZNF697 | 0.002536 |
| ARID3A | 0.002535 |
| MED25 | 0.002535 |
| CDK13 | 0.002535 |
| OR9A2 | 0.002535 |
| UQCC3 | 0.002534 |
| SLC26A8 | 0.002534 |
| ASH1L | 0.002533 |
| SNPH | 0.002533 |
| MAK | 0.002530 |
| XPO7 | 0.002529 |
| HDHD2 | 0.002529 |
| HS3ST6 | 0.002527 |
| STAMBPL1 | 0.002526 |
| OR51M1 | 0.002526 |
| PPM1N | 0.002526 |
| SHISA2 | 0.002526 |
| CMBL | 0.002526 |
| SYNCRIP | 0.002526 |
| MZT2B | 0.002526 |
| GPHB5 | 0.002525 |
| CCDC70 | 0.002525 |
| KNG1 | 0.002524 |
| CLEC4F | 0.002522 |
| FAM84B | 0.002522 |
| TMEM68 | 0.002521 |
| RFNG | 0.002521 |
| OARD1 | 0.002521 |
| FAM228B | 0.002517 |
| RGP1 | 0.002515 |
| TRAF4 | 0.002515 |
| BIRC6 | 0.002515 |
| GNMT | 0.002515 |
| SLC7A13 | 0.002515 |
| FAM156B | 0.002514 |
| PUS1 | 0.002514 |
| DYRK1B | 0.002514 |
| UBALD2 | 0.002512 |
| CACNA1H | 0.002511 |
| ADAD1 | 0.002511 |
| FAM126B | 0.002510 |
| GFOD2 | 0.002509 |
| DYNLT1 | 0.002509 |
| ALKBH1 | 0.002507 |
| POLR3D | 0.002506 |
| TIMM17B | 0.002506 |
| PCBD2 | 0.002505 |
| C5orf22 | 0.002505 |
| ACSF3 | 0.002505 |
| ZMYM6 | 0.002505 |
| MAFG | 0.002504 |
| CYLC2 | 0.002504 |
| MAGEB1 | 0.002504 |
| MORC1 | 0.002503 |
| OC90 | 0.002503 |
| LRTOMT | 0.002503 |
| AS3MT | 0.002502 |
| SRSF9 | 0.002501 |
| CD320 | 0.002501 |
| TMEM215 | 0.002501 |
| INTS3 | 0.002499 |
| CSNK1A1L | 0.002498 |
| MRPS33 | 0.002498 |
| OR10A2 | 0.002496 |
| CDC16 | 0.002494 |
| CPNE9 | 0.002493 |
| TIMM17A | 0.002493 |
| PLA2G12A | 0.002493 |
| KHDC3L | 0.002492 |
| FBXW9 | 0.002492 |
| C16orf46 | 0.002491 |
| CLLU1 | 0.002491 |
| ZNF416 | 0.002491 |
| AGXT2 | 0.002491 |
| LRRC56 | 0.002491 |
| TSPY1 | 0.002490 |
| RFPL4B | 0.002490 |
| KLHDC10 | 0.002490 |
| MYNN | 0.002489 |
| C7orf62 | 0.002489 |
| NUDT15 | 0.002489 |
| ZBTB8A | 0.002489 |
| G6PC2 | 0.002489 |
| CBFB | 0.002489 |
| MAP7 | 0.002488 |
| SLC17A9 | 0.002487 |
| EIF2AK2 | 0.002485 |
| TAMM41 | 0.002485 |
| LARP6 | 0.002483 |
| CNPY4 | 0.002482 |
| RAB11FIP4 | 0.002480 |
| CBLC | 0.002477 |
| THAP3 | 0.002477 |
| RERE | 0.002476 |
| PPWD1 | 0.002476 |
| GCNTI | 0.002476 |
| LZTR1 | 0.002475 |
| SCAI | 0.002474 |
| ABCC10 | 0.002474 |
| PLSCR5 | 0.002473 |
| TBX22 | 0.002473 |
| CATSPER4 | 0.002472 |
| GPRC6A | 0.002471 |
| SCMH1 | 0.002470 |
| ZNF462 | 0.002470 |
| ZNF691 | 0.002469 |
| C5orf51 | 0.002469 |
| SFT2D3 | 0.002467 |
| GK2 | 0.002467 |
| MBD6 | 0.002466 |
| LIN7A | 0.002466 |
| WDR7 | 0.002466 |
| STIP1 | 0.002466 |
| RPTOR | 0.002466 |
| KAT7 | 0.002465 |
| COPG2 | 0.002465 |
| TMEM242 | 0.002465 |
| TPP2 | 0.002464 |
| VEZF1 | 0.002464 |
| COX11 | 0.002464 |
| DCAF10 | 0.002463 |
| FAM172A | 0.002463 |
| IGLL1 | 0.002462 |
| IPPK | 0.002460 |
| VPS72 | 0.002460 |
| ULBP1 | 0.002459 |
| PNLDC1 | 0.002459 |
| FAM50A | 0.002456 |
| TRIM15 | 0.002456 |
| C12orf29 | 0.002455 |
| SERPINA10 | 0.002455 |
| CCDC38 | 0.002452 |
| ZNF75D | 0.002452 |
| KLHL8 | 0.002451 |
| SRSF4 | 0.002451 |
| ATP6V0A2 | 0.002450 |
| KRT77 | 0.002450 |
| OR51L1 | 0.002449 |
| EPM2A | 0.002448 |
| EXD2 | 0.002445 |
| PHACTR4 | 0.002443 |
| MECP2 | 0.002442 |
| HGS | 0.002441 |
| CARS2 | 0.002440 |
| PLCL2 | 0.002440 |
| SYMPK | 0.002439 |
| INIP | 0.002439 |
| ZNF555 | 0.002438 |
| CTC1 | 0.002437 |
| MUC7 | 0.002437 |
| FSD2 | 0.002436 |
| IRGM | 0.002435 |
| ZNF880 | 0.002435 |
| RAB24 | 0.002434 |
| COL24A1 | 0.002434 |
| TYR | 0.002433 |
| HMGA1 | 0.002432 |
| GPR101 | 0.002430 |
| SIRT1 | 0.002430 |
| SLC25A12 | 0.002429 |
| SPINK2 | 0.002429 |
| DZIP1L | 0.002429 |
| C19orf43 | 0.002429 |
| DOHH | 0.002429 |
| HHATL | 0.002429 |
| C16orf45 | 0.002429 |
| FBX030 | 0.002428 |
| ATP13A1 | 0.002428 |
| TBCA | 0.002428 |
| ACP6 | 0.002427 |
| CBLL1 | 0.002426 |
| FAM186B | 0.002426 |
| SMUG1 | 0.002425 |
| REXO1 | 0.002425 |
| DIP2B | 0.002423 |
| P4HA3 | 0.002423 |
| RAB40B | 0.002423 |
| TMEM17 | 0.002421 |
| UBE2M | 0.002421 |
| PPP1CC | 0.002420 |
| KAT6A | 0.002420 |
| BTBD10 | 0.002419 |
| NR1D2 | 0.002419 |
| C8orf46 | 0.002416 |
| KRTAP3-3 | 0.002415 |
| ABHD17B | 0.002413 |
| MAGEB2 | 0.002412 |
| UBE2J2 | 0.002412 |
| FAM162A | 0.002412 |
| PYGO2 | 0.002412 |
| ECHDC2 | 0.002411 |
| BPIFB2 | 0.002411 |
| EIF2B4 | 0.002411 |
| UBAC1 | 0.002410 |
| MSL3 | 0.002410 |
| DCDC2C | 0.002409 |
| KATNA1 | 0.002408 |
| GTF3C1 | 0.002407 |
| TRIB3 | 0.002407 |
| OR7A5 | 0.002406 |
| PFN4 | 0.002404 |
| RNF169 | 0.002404 |
| AP4B1 | 0.002404 |
| TMX1 | 0.002404 |
| CCDC89 | 0.002403 |
| TMEM57 | 0.002402 |
| COPS3 | 0.002401 |
| C15orf40 | 0.002400 |
| ZNF703 | 0.002399 |
| PURB | 0.002399 |
| AGTPBP1 | 0.002399 |
| HDGF | 0.002398 |
| ANKRD54 | 0.002398 |
| SLC25A45 | 0.002397 |
| ATPAF1 | 0.002397 |
| NPEPPS | 0.002397 |
| ZNF589 | 0.002395 |
| KMT2D | 0.002394 |
| MRPL37 | 0.002393 |
| FETUB | 0.002393 |
| CUTA | 0.002393 |
| ZNF646 | 0.002391 |
| GNL2 | 0.002391 |
| C20orf173 | 0.002391 |
| PRMT1 | 0.002390 |
| GOLGA8O | 0.002390 |
| SNRPG | 0.002390 |
| HAT1 | 0.002389 |
| MAT2A | 0.002389 |
| ENDOV | 0.002388 |
| PGBD3 | 0.002387 |
| BRCC3 | 0.002387 |
| CCDC59 | 0.002387 |
| EPHB3 | 0.002385 |
| COX8C | 0.002383 |
| DYM | 0.002383 |
| SOX13 | 0.002382 |
| NDUFA4L2 | 0.002382 |
| MANSC4 | 0.002382 |
| MTA3 | 0.002382 |
| PRRT4 | 0.002382 |
| MCM3AP | 0.002381 |
| TUFT1 | 0.002380 |
| DSC1 | 0.002379 |
| ZFAND1 | 0.002378 |
| TMEM246 | 0.002378 |
| VPS26B | 0.002378 |
| PSG6 | 0.002377 |
| OTOP2 | 0.002375 |
| CCDC182 | 0.002375 |
| UGT3A2 | 0.002375 |
| NUDCD2 | 0.002375 |
| CES4A | 0.002374 |
| USP11 | 0.002374 |
| UBR4 | 0.002373 |
| ZNF596 | 0.002372 |
| TAC4 | 0.002371 |
| TMEM25 | 0.002371 |
| ZNF768 | 0.002371 |
| MALT1 | 0.002371 |
| RALGAPA2 | 0.002371 |
| SMIM10L1 | 0.002370 |
| WAC | 0.002370 |
| CALR3 | 0.002369 |
| RAD9B | 0.002369 |
| KANSL1L | 0.002368 |
| TIMM13 | 0.002368 |
| ATP5L2 | 0.002368 |
| POU2F2 | 0.002367 |
| SENP6 | 0.002365 |
| PPIL2 | 0.002365 |
| CMIP | 0.002365 |
| PPP1R9A | 0.002364 |
| KRTAP3-2 | 0.002362 |
| EPB41L4B | 0.002362 |
| ZNF16 | 0.002361 |
| PUM2 | 0.002360 |
| H3F3A | 0.002360 |
| PRH2 | 0.002359 |
| TEAD1 | 0.002359 |
| CDK12 | 0.002358 |
| SLC15A5 | 0.002356 |
| CNOT2 | 0.002354 |
| DEFB103A | 0.002354 |
| SHROOM1 | 0.002353 |
| SLC6A7 | 0.002353 |
| TCF23 | 0.002353 |
| SSTR3 | 0.002352 |
| FAM169B | 0.002352 |
| ZCCHC14 | 0.002351 |
| N6AMT1 | 0.002351 |
| ZNF846 | 0.002351 |
| DIEXF | 0.002350 |
| RBM17 | 0.002350 |
| ANKRD16 | 0.002349 |
| KRT81 | 0.002347 |
| ZNF19 | 0.002347 |
| CNTNAP3 | 0.002344 |
| ACOT11 | 0.002344 |
| ABCA7 | 0.002343 |
| CREB1 | 0.002343 |
| PKP4 | 0.002342 |
| MORC3 | 0.002342 |
| DNAJC11 | 0.002341 |
| MRPL41 | 0.002341 |
| TMEM39B | 0.002341 |
| MRRF | 0.002341 |
| USP46 | 0.002340 |
| CCDC158 | 0.002338 |
| ZNF561 | 0.002337 |
| SRGAP2 | 0.002337 |
| TDRD1 | 0.002335 |
| KPRP | 0.002335 |
| UTP23 | 0.002335 |
| AP5Z1 | 0.002335 |
| PARP6 | 0.002335 |
| NELFA | 0.002334 |
| NRL | 0.002333 |
| GAGE12J | 0.002330 |
| HYDIN | 0.002330 |
| GAB4 | 0.002328 |
| USP22 | 0.002327 |
| ASTE1 | 0.002326 |
| TOP2B | 0.002325 |
| OR52A5 | 0.002325 |
| PKN1 | 0.002325 |
| MT-C03 | 0.002324 |
| UBQLN3 | 0.002324 |
| MAEL | 0.002324 |
| IPO13 | 0.002323 |
| FRA10AC1 | 0.002321 |
| TRMT61A | 0.002321 |
| ADAM30 | 0.002321 |
| ARFGEF1 | 0.002320 |
| APOA4 | 0.002320 |
| PLIN4 | 0.002319 |
| MED6 | 0.002319 |
| RBPJL | 0.002319 |
| LETMD1 | 0.002319 |
| C1QTNF8 | 0.002318 |
| HDAC8 | 0.002318 |
| MED9 | 0.002318 |
| WDR20 | 0.002318 |
| NPY2R | 0.002318 |
| MRPS21 | 0.002316 |
| OR52H1 | 0.002315 |
| MUC2 | 0.002315 |
| GPS1 | 0.002315 |
| IPO4 | 0.002315 |
| GPD2 | 0.002315 |
| MC3R | 0.002314 |
| KBTBD7 | 0.002314 |
| KRTAP10-3 | 0.002313 |
| INPP4A | 0.002313 |
| AHDC1 | 0.002313 |
| SLC39A10 | 0.002313 |
| TLR9 | 0.002312 |
| SCNM1 | 0.002312 |
| DRD3 | 0.002312 |
| CHAMP1 | 0.002311 |
| ATP2B1 | 0.002311 |
| PUM1 | 0.002309 |
| FUNDC1 | 0.002309 |
| COL5A3 | 0.002308 |
| OR8B2 | 0.002308 |
| DHX37 | 0.002307 |
| LYPD6B | 0.002307 |
| TATDN1 | 0.002307 |
| HIST3H3 | 0.002306 |
| FRG2C | 0.002306 |
| FAM220A | 0.002306 |
| FAM127B | 0.002305 |
| PRKD1 | 0.002305 |
| ZSCAN5B | 0.002305 |
| DCAF7 | 0.002305 |
| MAPK10 | 0.002304 |
| TRIM49 | 0.002304 |
| PPP2R5E | 0.002304 |
| TBL1X | 0.002303 |
| KRTAP10-9 | 0.002303 |
| OSBPL7 | 0.002302 |
| ERCC3 | 0.002302 |
| NKAIN4 | 0.002301 |
| URI1 | 0.002301 |
| RHEB | 0.002300 |
| KPNA1 | 0.002299 |
| RLBP1 | 0.002299 |
| UBXN2A | 0.002299 |
| DEFB103B | 0.002299 |
| TGM6 | 0.002298 |
| GORAB | 0.002297 |
| TTC37 | 0.002297 |
| CARD18 | 0.002296 |
| OR52B2 | 0.002296 |
| RGPD6 | 0.002295 |
| ANKRD17 | 0.002295 |
| RAB3GAP2 | 0.002294 |
| PPP1R14D | 0.002294 |
| ABCB4 | 0.002294 |
| MTRNR2L6 | 0.002293 |
| SNAP47 | 0.002292 |
| IFT20 | 0.002290 |
| PRR34 | 0.002290 |
| OR11L1 | 0.002289 |
| RAP2A | 0.002288 |
| FAM153A | 0.002288 |
| ALDOB | 0.002287 |
| HCFC2 | 0.002287 |
| C8orf76 | 0.002286 |
| ARL3 | 0.002285 |
| PITHD1 | 0.002285 |
| DMXL1 | 0.002284 |
| TRAPPC9 | 0.002284 |
| SCAPER | 0.002283 |
| NOC3L | 0.002283 |
| ZC3H14 | 0.002283 |
| SLC22A25 | 0.002283 |
| HYPM | 0.002282 |
| THEGL | 0.002281 |
| PABPC3 | 0.002280 |
| METTL2A | 0.002280 |
| BSPRY | 0.002280 |
| SUPV3L1 | 0.002280 |
| KLHDC2 | 0.002279 |
| CABS1 | 0.002279 |
| FBXO47 | 0.002279 |
| SMYD2 | 0.002278 |
| MCUR1 | 0.002278 |
| CXorf40B | 0.002278 |
| YY1 | 0.002277 |
| SOX10 | 0.002277 |
| ABCB11 | 0.002276 |
| CTAGE1 | 0.002276 |
| SH3PXD2A | 0.002274 |
| NDRG3 | 0.002274 |
| AOC2 | 0.002273 |
| XRRA1 | 0.002273 |
| SOS2 | 0.002273 |
| HTN3 | 0.002272 |
| RBM18 | 0.002271 |
| ULK2 | 0.002270 |
| MDP1 | 0.002270 |
| C6orf52 | 0.002270 |
| BAG4 | 0.002270 |
| USP36 | 0.002269 |
| ZBTB49 | 0.002269 |
| RBM12 | 0.002268 |
| KDM4C | 0.002268 |
| KLHL22 | 0.002267 |
| FAM212B | 0.002267 |
| POTEB3 | 0.002266 |
| C2orf68 | 0.002266 |
| LETM2 | 0.002266 |
| BBS10 | 0.002266 |
| LGI1 | 0.002266 |
| IL5 | 0.002265 |
| LACE1 | 0.002264 |
| TBC1D12 | 0.002264 |
| ICK | 0.002264 |
| NDUFB11 | 0.002263 |
| CNNM2 | 0.002263 |
| MS4A18 | 0.002263 |
| C12orf45 | 0.002260 |
| SOX3 | 0.002260 |
| CMC4 | 0.002259 |
| DNAJC27 | 0.002258 |
| KLHL12 | 0.002258 |
| IFITM5 | 0.002257 |
| TMTC3 | 0.002256 |
| NOL10 | 0.002255 |
| CASC3 | 0.002255 |
| IFNL4 | 0.002254 |
| MAGOH | 0.002254 |
| OR4F17 | 0.002253 |
| TAT | 0.002252 |
| MORN3 | 0.002252 |
| CDC27 | 0.002251 |
| SPDYE3 | 0.002250 |
| ANKS4B | 0.002249 |
| TRPM7 | 0.002249 |
| ZNF205 | 0.002246 |
| ATG14 | 0.002244 |
| RAX2 | 0.002244 |
| SMARCE1 | 0.002244 |
| C19orf47 | 0.002243 |
| TPGS2 | 0.002243 |
| FAAP20 | 0.002242 |
| TGDS | 0.002242 |
| CLOCK | 0.002242 |
| NME2 | 0.002242 |
| RAB19 | 0.002242 |
| CSNK1G1 | 0.002241 |
| ABCC12 | 0.002241 |
| ROR2 | 0.002241 |
| PAAF1 | 0.002241 |
| TOPAZ1 | 0.002240 |
| PAPOLG | 0.002239 |
| RPF2 | 0.002239 |
| ME2 | 0.002239 |
| LEF1 | 0.002239 |
| C16orf95 | 0.002238 |
| FAM110B | 0.002237 |
| GPN2 | 0.002235 |
| CBX4 | 0.002233 |
| POLR2K | 0.002233 |
| MYL5 | 0.002233 |
| CILP2 | 0.002232 |
| CYB5R4 | 0.002231 |
| FOXD4L6 | 0.002230 |
| AMY2A | 0.002230 |
| GLTPD2 | 0.002230 |
| UCP1 | 0.002229 |
| LGR5 | 0.002228 |
| ATXN7L1 | 0.002227 |
| RPUSD1 | 0.002227 |
| TRPV3 | 0.002227 |
| STX17 | 0.002226 |
| ARL5C | 0.002226 |
| PPID | 0.002226 |
| C6orf203 | 0.002225 |
| ZNF414 | 0.002223 |
| GLYCTK | 0.002221 |
| CHKA | 0.002221 |
| NSUN3 | 0.002220 |
| RPUSD4 | 0.002220 |
| RMND1 | 0.002220 |
| KDM4D | 0.002220 |
| DNAL1 | 0.002218 |
| PRKACB | 0.002217 |
| SPATA31D4 | 0.002217 |
| KAT6B | 0.002217 |
| ANO7 | 0.002217 |
| YRDC | 0.002216 |
| ZNF645 | 0.002216 |
| RECQL | 0.002215 |
| FAM91A1 | 0.002215 |
| VIPAS39 | 0.002215 |
| PPIL1 | 0.002214 |
| JMJD6 | 0.002214 |
| OR56A4 | 0.002213 |
| UBE2I | 0.002212 |
| GADD45GIP1 | 0.002210 |
| OR10A4 | 0.002209 |
| DECR2 | 0.002208 |
| DACH1 | 0.002203 |
| MFSD3 | 0.002202 |
| ZDHHC14 | 0.002202 |
| ZNF98 | 0.002201 |
| CCDC134 | 0.002201 |
| FABP2 | 0.002200 |
| R3HDM2 | 0.002199 |
| HIST1H2BC | 0.002199 |
| CASP14 | 0.002198 |
| FNIP1 | 0.002198 |
| C2orf16 | 0.002197 |
| SLC22A11 | 0.002197 |
| NEK4 | 0.002197 |
| LRRCC1 | 0.002196 |
| LYRM4 | 0.002196 |
| PHF3 | 0.002195 |
| PRRT1 | 0.002195 |
| STAR | 0.002195 |
| NUCB2 | 0.002195 |
| MED13 | 0.002195 |
| SLC25A51 | 0.002194 |
| C11orf57 | 0.002193 |
| NIT2 | 0.002192 |
| FN3KRP | 0.002191 |
| SLC4A9 | 0.002191 |
| NARFL | 0.002190 |
| GDAP2 | 0.002190 |
| CXorf67 | 0.002189 |
| MED12 | 0.002188 |
| TDP2 | 0.002186 |
| RBM4 | 0.002186 |
| FAM231C | 0.002185 |
| PAM16 | 0.002185 |
| DTWD1 | 0.002184 |
| TFDP3 | 0.002182 |
| DGAT2L6 | 0.002182 |
| ZBTB34 | 0.002182 |
| ALOX12 | 0.002181 |
| THNSL1 | 0.002181 |
| CCER1 | 0.002181 |
| SULT1C3 | 0.002180 |
| HSPA6 | 0.002179 |
| PIM1 | 0.002179 |
| ASIP | 0.002178 |
| NUAK2 | 0.002178 |
| PHC3 | 0.002178 |
| PYY | 0.002177 |
| PPP6R3 | 0.002177 |
| ZSWEM1 | 0.002177 |
| CKM | 0.002177 |
| VPS52 | 0.002177 |
| FOXD4L1 | 0.002177 |
| C10orf53 | 0.002177 |
| LDB3 | 0.002176 |
| NKIRAS2 | 0.002176 |
| MAN2C1 | 0.002176 |
| ZNF717 | 0.002175 |
| 43719 | 0.002175 |
| MAEA | 0.002175 |
| FXYD2 | 0.002175 |
| SNW1 | 0.002175 |
| TAS2R7 | 0.002175 |
| TBC1D3D | 0.002173 |
| YBEY | 0.002173 |
| ITPKA | 0.002172 |
| PSME4 | 0.002172 |
| TMEM18 | 0.002171 |
| RXRB | 0.002170 |
| FOXS1 | 0.002168 |
| MTHFSD | 0.002168 |
| NPFFR2 | 0.002167 |
| METTL18 | 0.002166 |
| IREB2 | 0.002165 |
| SLX1A | 0.002165 |
| CNOT6L | 0.002164 |
| ERAL1 | 0.002162 |
| COPS8 | 0.002162 |
| TCEA2 | 0.002162 |
| PRAMEF1 | 0.002161 |
| OR9K2 | 0.002161 |
| C12orf4 | 0.002161 |
| CST4 | 0.002161 |
| ZNF720 | 0.002160 |
| TSSK1B | 0.002159 |
| MIOS | 0.002158 |
| C9 | 0.002158 |
| CAPRIN1 | 0.002158 |
| MRTO4 | 0.002158 |
| TTC34 | 0.002158 |
| ZFP91 | 0.002157 |
| ZNF468 | 0.002155 |
| HSPA2 | 0.002155 |
| ZNF813 | 0.002154 |
| SPATA32 | 0.002153 |
| OR5H8 | 0.002153 |
| ARNT2 | 0.002153 |
| CFAP69 | 0.002153 |
| PRKAR2B | 0.002152 |
| FAM47A | 0.002152 |
| OR6B2 | 0.002151 |
| SUMO2 | 0.002151 |
| CRACR2A | 0.002149 |
| GOLGA8R | 0.002148 |
| KCNK4 | 0.002148 |
| RIPPLY1 | 0.002148 |
| TMEM185A | 0.002148 |
| PRMT5 | 0.002148 |
| WBP2NL | 0.002147 |
| PET117 | 0.002147 |
| TIFA | 0.002146 |
| POTEM | 0.002144 |
| WDR36 | 0.002142 |
| CHM | 0.002141 |
| L3HYPDH | 0.002141 |
| CAMKK1 | 0.002140 |
| KRTAP12-2 | 0.002139 |
| KMT2E | 0.002138 |
| ZNF492 | 0.002137 |
| DCTN4 | 0.002136 |
| C11orf95 | 0.002136 |
| ZNF583 | 0.002136 |
| UBE2N | 0.002134 |
| RBBP8NL | 0.002134 |
| POU3F4 | 0.002133 |
| TUT1 | 0.002132 |
| WISP1 | 0.002131 |
| SF3B3 | 0.002131 |
| ZFAT | 0.002131 |
| ELFN1 | 0.002131 |
| COX7A2 | 0.002130 |
| DNAH8 | 0.002129 |
| USP7 | 0.002128 |
| MYLK4 | 0.002128 |
| SDHAF4 | 0.002128 |
| IL17B | 0.002127 |
| SEMA4C | 0.002126 |
| MRPS22 | 0.002125 |
| UBE2Q2L | 0.002125 |
| TUBG2 | 0.002124 |
| ZFYVE27 | 0.002123 |
| UBE2G2 | 0.002122 |
| ASCL3 | 0.002122 |
| AKAP1 | 0.002120 |
| RHOBTB3 | 0.002119 |
| ACTL7A | 0.002119 |
| MRPS14 | 0.002119 |
| SEMA4D | 0.002119 |
| COQ4 | 0.002119 |
| ERCC8 | 0.002118 |
| KRTAP10-5 | 0.002116 |
| TMEM175 | 0.002115 |
| OR51F1 | 0.002115 |
| TRIM44 | 0.002114 |
| METTL6 | 0.002114 |
| ELAC1 | 0.002114 |
| KRTAP10-1 | 0.002114 |
| TRIM54 | 0.002113 |
| RPA2 | 0.002113 |
| KCNK7 | 0.002112 |
| POLR1B | 0.002111 |
| KIAA1429 | 0.002111 |
| TCAF1 | 0.002110 |
| MED19 | 0.002110 |
| FH | 0.002109 |
| LEP | 0.002108 |
| HNRNPLL | 0.002108 |
| NLRP9 | 0.002107 |
| TMPRSS11B | 0.002105 |
| HNRNPA1 | 0.002104 |
| CRIPAK | 0.002103 |
| NUP88 | 0.002103 |
| CCDC169 | 0.002103 |
| C14orf105 | 0.002103 |
| UGGT2 | 0.002102 |
| CLCC1 | 0.002101 |
| USP17L15 | 0.002101 |
| KANSL3 | 0.002101 |
| HSBP1 | 0.002101 |
| COG3 | 0.002100 |
| NDUFB4 | 0.002100 |
| ZNF613 | 0.002100 |
| NOBOX | 0.002099 |
| CAAP1 | 0.002098 |
| PPMIB | 0.002098 |
| NSL1 | 0.002097 |
| CDKN2AIP | 0.002096 |
| EPC1 | 0.002096 |
| GABRA2 | 0.002095 |
| FAM219B | 0.002095 |
| MRPL36 | 0.002094 |
| CRYZL1 | 0.002094 |
| VSX2 | 0.002093 |
| C6orf58 | 0.002093 |
| ZNF384 | 0.002093 |
| N4BP2L2 | 0.002092 |
| DDX23 | 0.002092 |
| UBR3 | 0.002092 |
| NAP1L1 | 0.002090 |
| PMCH | 0.002089 |
| MCCD1 | 0.002089 |
| RARS2 | 0.002088 |
| TXNDC8 | 0.002087 |
| OR2A2 | 0.002087 |
| POPS | 0.002086 |
| CPNE5 | 0.002085 |
| SATB1 | 0.002085 |
| OR1J2 | 0.002084 |
| H2AFB2 | 0.002083 |
| OR1L1 | 0.002083 |
| ESX1 | 0.002083 |
| DDX20 | 0.002082 |
| OR2K2 | 0.002082 |
| LYPD4 | 0.002082 |
| UBE2K | 0.002081 |
| TARS2 | 0.002081 |
| PIP5K1A | 0.002079 |
| HIR3E | 0.002079 |
| KCP | 0.002078 |
| BLCAP | 0.002078 |
| TPRX1 | 0.002078 |
| CHD1 | 0.002077 |
| GOLGA8H | 0.002077 |
| CEP68 | 0.002077 |
| WRB | 0.002076 |
| PPP1R8 | 0.002076 |
| WDFY3 | 0.002076 |
| MRPL13 | 0.002075 |
| IRAKI | 0.002072 |
| ZNF433 | 0.002072 |
| NDUFS6 | 0.002071 |
| SYTL1 | 0.002071 |
| HACD3 | 0.002071 |
| AHCY | 0.002070 |
| ZIM2 | 0.002070 |
| CST2 | 0.002069 |
| DYIN | 0.002068 |
| PGS1 | 0.002068 |
| GPLD1 | 0.002068 |
| DGAT2 | 0.002067 |
| CLDND1 | 0.002064 |
| NKX2-3 | 0.002064 |
| TAF6 | 0.002063 |
| ZNF148 | 0.002063 |
| MRPL9 | 0.002059 |
| SCN11A | 0.002058 |
| CRCP | 0.002057 |
| ASB6 | 0.002057 |
| SPAM1 | 0.002054 |
| GLRX3 | 0.002054 |
| CSTF1 | 0.002054 |
| MLLT6 | 0.002053 |
| OR4K1 | 0.002053 |
| TRAPPCI3 | 0.002052 |
| ZNF781 | 0.002051 |
| OR1L6 | 0.002051 |
| HIPK2 | 0.002051 |
| OR2W5 | 0.002051 |
| OXLD1 | 0.002051 |
| TMEM255B | 0.002050 |
| MEA1 | 0.002050 |
| CTCF | 0.002050 |
| SLC23A2 | 0.002049 |
| MOAP1 | 0.002048 |
| SGTB | 0.002047 |
| SF3A3 | 0.002047 |
| PQBP1 | 0.002046 |
| CHD2 | 0.002045 |
| NDUFA11 | 0.002045 |
| DNAH1 | 0.002043 |
| RCBTB1 | 0.002043 |
| C2orf80 | 0.002043 |
| TOPORS | 0.002042 |
| PTTG2 | 0.002041 |
| CLPSL1 | 0.002041 |
| ADAD2 | 0.002041 |
| RNF151 | 0.002040 |
| DDK | 0.002040 |
| PAK1IP1 | 0.002039 |
| TMX3 | 0.002037 |
| FANCD2OS | 0.002036 |
| BYSL | 0.002036 |
| SKAP1 | 0.002035 |
| CTXN3 | 0.002035 |
| SUGT1 | 0.002034 |
| ARL6 | 0.002033 |
| RBM27 | 0.002033 |
| NUBP2 | 0.002032 |
| PTX4 | 0.002031 |
| MAK16 | 0.002030 |
| C17orf62 | 0.002029 |
| TCEAL2 | 0.002029 |
| DIABLO | 0.002028 |
| FAM98B | 0.002028 |
| FASTK | 0.002026 |
| ETNK1 | 0.002025 |
| OSBPL2 | 0.002024 |
| DDX42 | 0.002023 |
| DYNAP | 0.002023 |
| OR52E4 | 0.002022 |
| DCAF12L1 | 0.002021 |
| CALML4 | 0.002021 |
| RAET1L | 0.002021 |
| HNRNPUL1 | 0.002021 |
| ASCL2 | 0.002020 |
| PSMC1 | 0.002020 |
| CACNA1D | 0.002019 |
| COX5B | 0.002018 |
| SLC13A1 | 0.002018 |
| OTUB2 | 0.002017 |
| TEX11 | 0.002016 |
| CYC1 | 0.002015 |
| ZNF799 | 0.002015 |
| CKMT2 | 0.002013 |
| CSNK1G3 | 0.002013 |
| DAXX | 0.002012 |
| BOLA3 | 0.002011 |
| API5 | 0.002011 |
| SENP2 | 0.002009 |
| SNRPB2 | 0.002008 |
| PHKG2 | 0.002008 |
| PRR23B | 0.002007 |
| WDR45B | 0.002006 |
| ANHX | 0.002006 |
| TMEM108 | 0.002006 |
| PCMT1 | 0.002006 |
| SLC9A2 | 0.002006 |
| GPS2 | 0.002005 |
| SLC25A42 | 0.002005 |
| TFCP2 | 0.002003 |
| CHD1L | 0.002002 |
| SETMAR | 0.002002 |
| MC5R | 0.002001 |
| TUBD1 | 0.002000 |
| LYRM2 | 0.001999 |
| C11orf49 | 0.001998 |
| CIRBP | 0.001998 |
| LRRN1 | 0.001998 |
| NFYB | 0.001998 |
| SLC8A3 | 0.001995 |
| DIS3L | 0.001995 |
| SYT17 | 0.001994 |
| SFXN2 | 0.001992 |
| NADK2 | 0.001992 |
| SLC26A11 | 0.001991 |
| FAM20B | 0.001990 |
| GGNBP2 | 0.001989 |
| ALG1L2 | 0.001988 |
| THBS4 | 0.001988 |
| NDUFA10 | 0.001987 |
| TTC9C | 0.001986 |
| RYK | 0.001986 |
| GAPDHS | 0.001986 |
| NADSYN1 | 0.001986 |
| ZNF57 | 0.001985 |
| C14orf180 | 0.001984 |
| RPF1 | 0.001984 |
| SMAD2 | 0.001983 |
| HARS2 | 0.001982 |
| ZNF358 | 0.001982 |
| SETD1A | 0.001982 |
| TOR2A | 0.001982 |
| SCN4A | 0.001982 |
| C17orf74 | 0.001980 |
| RAD51AP2 | 0.001979 |
| GATAD1 | 0.001977 |
| CLDN12 | 0.001977 |
| 43529 | 0.001976 |
| SHCBP1L | 0.001976 |
| ANGEL1 | 0.001976 |
| ZNF804A | 0.001976 |
| IRF2BP1 | 0.001975 |
| RB1CC1 | 0.001975 |
| MLST8 | 0.001973 |
| PDXDC1 | 0.001973 |
| MFSD8 | 0.001972 |
| KRTAP1-5 | 0.001971 |
| BPIFB4 | 0.001971 |
| GTF2IRD2 | 0.001971 |
| CERS5 | 0.001971 |
| TMEM229B | 0.001971 |
| ABCE1 | 0.001971 |
| KCNA6 | 0.001970 |
| ABCB8 | 0.001970 |
| VRTN | 0.001970 |
| TMEM257 | 0.001969 |
| HNRNPA0 | 0.001969 |
| OR52N1 | 0.001968 |
| HSP90B1 | 0.001968 |
| BCKDHB | 0.001968 |
| SNRPC | 0.001967 |
| IRAK4 | 0.001966 |
| CCDC122 | 0.001965 |
| KRT2 | 0.001965 |
| PDHA1 | 0.001963 |
| ZNF444 | 0.001963 |
| ZCCHC3 | 0.001963 |
| ZNF396 | 0.001960 |
| FAM196B | 0.001959 |
| ATG12 | 0.001957 |
| B9D1 | 0.001957 |
| NDUFS5 | 0.001957 |
| OR8B8 | 0.001956 |
| SHTN1 | 0.001955 |
| PYDC1 | 0.001954 |
| NAALADL1 | 0.001954 |
| GIN1 | 0.001954 |
| ZSCAN25 | 0.001954 |
| AP1S1 | 0.001952 |
| COX7B | 0.001951 |
| RSBN1 | 0.001951 |
| GPR37 | 0.001951 |
| SEC61G | 0.001950 |
| OR2A5 | 0.001950 |
| OR2L8 | 0.001949 |
| TBC1D22B | 0.001949 |
| ZNF654 | 0.001949 |
| FAM110A | 0.001948 |
| LIN7C | 0.001948 |
| TLE2 | 0.001948 |
| C8orf74 | 0.001948 |
| SMARCA1 | 0.001947 |
| OR13C2 | 0.001946 |
| UBE4B | 0.001946 |
| SIPA1L3 | 0.001945 |
| GBAS | 0.001944 |
| IFT52 | 0.001944 |
| GSC2 | 0.001943 |
| PIGU | 0.001943 |
| CLEC2D | 0.001943 |
| AP3D1 | 0.001943 |
| ID2 | 0.001943 |
| BEX4 | 0.001942 |
| CSH1 | 0.001940 |
| SPINK8 | 0.001940 |
| NLRP14 | 0.001940 |
| MRPS5 | 0.001939 |
| GPBP1 | 0.001937 |
| RRP15 | 0.001937 |
| PRPH2 | 0.001934 |
| TMX4 | 0.001934 |
| HMGN3 | 0.001934 |
| CEACAM20 | 0.001933 |
| MDGA1 | 0.001933 |
| TRIM13 | 0.001932 |
| OR2A12 | 0.001931 |
| PSMB11 | 0.001931 |
| ZSWIM3 | 0.001930 |
| EXOSC6 | 0.001928 |
| NOP9 | 0.001926 |
| NMT2 | 0.001926 |
| PDYN | 0.001926 |
| SCYL2 | 0.001926 |
| PARP11 | 0.001926 |
| KLHL40 | 0.001925 |
| PDE6D | 0.001925 |
| AASDHPPT | 0.001925 |
| USP13 | 0.001924 |
| LCA5 | 0.001923 |
| PLEKHA5 | 0.001921 |
| IFNB1 | 0.001918 |
| MRPL21 | 0.001917 |
| LSM14A | 0.001917 |
| RPL22L1 | 0.001916 |
| MKLN1 | 0.001915 |
| LRRC73 | 0.001914 |
| ZNF576 | 0.001913 |
| SUPT5H | 0.001913 |
| ZGPAT | 0.001912 |
| TMC2 | 0.001912 |
| ATP6AP1L | 0.001912 |
| TXLNB | 0.001911 |
| IPO8 | 0.001911 |
| ZNF584 | 0.001911 |
| TTC24 | 0.001909 |
| SPIN2B | 0.001909 |
| XAB2 | 0.001909 |
| BMP10 | 0.001909 |
| ZNF830 | 0.001908 |
| PFKM | 0.001908 |
| NPEPL1 | 0.001907 |
| SPATA31A6 | 0.001906 |
| ISG20L2 | 0.001906 |
| PDZD9 | 0.001905 |
| APOOL | 0.001905 |
| SPATA31A3 | 0.001905 |
| SPATA8 | 0.001904 |
| MAGEB10 | 0.001903 |
| GPANK1 | 0.001903 |
| BCCIP | 0.001902 |
| FGF14 | 0.001902 |
| PTH | 0.001901 |
| PDCD11 | 0.001900 |
| NEIL1 | 0.001899 |
| OR11H1 | 0.001898 |
| KIDINS220 | 0.001897 |
| CGGBP1 | 0.001897 |
| SYNRG | 0.001897 |
| OR2A25 | 0.001896 |
| CEP126 | 0.001895 |
| EHBP1 | 0.001894 |
| SMOX | 0.001894 |
| DHRS7C | 0.001894 |
| TGIF2LY | 0.001893 |
| CCAR2 | 0.001892 |
| SLC30A4 | 0.001892 |
| CMYA5 | 0.001892 |
| FEM1B | 0.001891 |
| PHRF1 | 0.001891 |
| SERBP1 | 0.001889 |
| KCNG4 | 0.001888 |
| RGL2 | 0.001888 |
| FUBP3 | 0.001887 |
| INTS6 | 0.001887 |
| POLE3 | 0.001887 |
| RPL36A | 0.001887 |
| THOC6 | 0.001886 |
| LAG3 | 0.001886 |
| OR13C5 | 0.001886 |
| RAD23B | 0.001886 |
| NUTM1 | 0.001885 |
| SLC29A2 | 0.001885 |
| ABCB10 | 0.001885 |
| UBXN4 | 0.001885 |
| AP1S2 | 0.001885 |
| EAF2 | 0.001885 |
| ATP8B2 | 0.001883 |
| HSD17B7 | 0.001883 |
| HEMK1 | 0.001882 |
| TAAR6 | 0.001882 |
| BRD4 | 0.001881 |
| PIGH | 0.001880 |
| ITGA11 | 0.001880 |
| GPX7 | 0.001880 |
| PDHA2 | 0.001879 |
| MPC2 | 0.001879 |
| ACOT7 | 0.001879 |
| CXADR | 0.001879 |
| PDCD10 | 0.001878 |
| NT5C3B | 0.001878 |
| OR4F16 | 0.001878 |
| OR4F29 | 0.001878 |
| PIK3CA | 0.001878 |
| CUL5 | 0.001878 |
| DYNLL2 | 0.001877 |
| CPO | 0.001874 |
| NIPSNAP3A | 0.001874 |
| SPINK7 | 0.001873 |
| RPRD2 | 0.001872 |
| SSB | 0.001872 |
| REXO4 | 0.001872 |
| OR1N1 | 0.001872 |
| DCTPP1 | 0.001871 |
| XRN2 | 0.001871 |
| TRAPPC10 | 0.001871 |
| BCDIN3D | 0.001870 |
| FECH | 0.001867 |
| KRTAP11-1 | 0.001867 |
| CXorf49 | 0.001867 |
| EPS15L1 | 0.001866 |
| DPH5 | 0.001866 |
| SCOC | 0.001865 |
| C2orf83 | 0.001864 |
| EXOC3 | 0.001864 |
| ETNPPL | 0.001863 |
| GDF6 | 0.001863 |
| MRPS25 | 0.001862 |
| ZSCAN5A | 0.001861 |
| FLCN | 0.001860 |
| GOLGA8M | 0.001860 |
| SEMA6D | 0.001859 |
| LYRM7 | 0.001857 |
| ANKRD9 | 0.001857 |
| OR6V1 | 0.001857 |
| FBXO40 | 0.001857 |
| C4orf51 | 0.001856 |
| GLRX2 | 0.001854 |
| MSANTD4 | 0.001854 |
| HMGB1 | 0.001854 |
| ZNF784 | 0.001852 |
| OR4M1 | 0.001852 |
| CFAP36 | 0.001852 |
| IHΥN1 | 0.001852 |
| GLE1 | 0.001851 |
| DCHS1 | 0.001851 |
| EGFLAM | 0.001850 |
| HCAR1 | 0.001850 |
| BLOC1S5 | 0.001849 |
| GSKIP | 0.001849 |
| PANX1 | 0.001849 |
| AAGAB | 0.001848 |
| OR2J2 | 0.001847 |
| CCDC127 | 0.001847 |
| ZNF865 | 0.001846 |
| USP16 | 0.001845 |
| POLG | 0.001845 |
| MIA3 | 0.001845 |
| AMELX | 0.001844 |
| TAF15 | 0.001844 |
| SMTN | 0.001843 |
| ATP5S | 0.001842 |
| IRF2BPL | 0.001842 |
| ARFGEF2 | 0.001842 |
| DPMI | 0.001841 |
| C21orf2 | 0.001840 |
| TYW1 | 0.001840 |
| PNLIPRP1 | 0.001840 |
| BPIFA3 | 0.001840 |
| CFC1B | 0.001840 |
| FAM104B | 0.001839 |
| CALM3 | 0.001839 |
| SCRN1 | 0.001838 |
| AARSD1 | 0.001837 |
| ELMOD3 | 0.001837 |
| OR10K1 | 0.001836 |
| C16orf70 | 0.001835 |
| PTGES3 | 0.001834 |
| EGLN1 | 0.001832 |
| CDC40 | 0.001832 |
| KRTAP10-8 | 0.001832 |
| INSL6 | 0.001831 |
| MRGPRD | 0.001831 |
| L3MBTL3 | 0.001830 |
| FBX03 | 0.001829 |
| CNOT10 | 0.001826 |
| ZYGIIB | 0.001826 |
| CRYBAI | 0.001825 |
| PMPCA | 0.001825 |
| ACAT2 | 0.001824 |
| LARP4 | 0.001824 |
| AMMECR1 | 0.001822 |
| KRTAP10-12 | 0.001822 |
| ANAPC11 | 0.001821 |
| CAMKMT | 0.001820 |
| HINT2 | 0.001819 |
| POM121 | 0.001818 |
| ZNF619 | 0.001818 |
| FOCAD | 0.001818 |
| MYOT | 0.001817 |
| OR4N2 | 0.001815 |
| OTOL1 | 0.001815 |
| BUD13 | 0.001813 |
| STK38 | 0.001813 |
| PSMD14 | 0.001812 |
| EZH1 | 0.001812 |
| OR2M3 | 0.001810 |
| ZFP64 | 0.001810 |
| CSN2 | 0.001809 |
| ATRIP | 0.001808 |
| MRPL52 | 0.001808 |
| ZIM3 | 0.001806 |
| HACD2 | 0.001806 |
| HPS3 | 0.001806 |
| YIPF6 | 0.001804 |
| NFE2L3 | 0.001804 |
| C22orf39 | 0.001803 |
| SRP54 | 0.001803 |
| TRNT1 | 0.001802 |
| SGCB | 0.001801 |
| FAM71B | 0.001801 |
| LNP1 | 0.001798 |
| AGGF1 | 0.001798 |
| DSG4 | 0.001797 |
| TRIM23 | 0.001797 |
| MRPL50 | 0.001797 |
| N4BP3 | 0.001796 |
| VN1R2 | 0.001796 |
| ABHD17A | 0.001795 |
| EID3 | 0.001794 |
| FAM135A | 0.001794 |
| C17orf51 | 0.001793 |
| CHCHD2 | 0.001791 |
| ZNF788 | 0.001790 |
| UBTD2 | 0.001788 |
| FAIM | 0.001788 |
| RUVBL2 | 0.001788 |
| TGFBRAP1 | 0.001786 |
| DDX31 | 0.001785 |
| AGL | 0.001784 |
| PLEK2 | 0.001784 |
| TASP1 | 0.001783 |
| GABRA4 | 0.001783 |
| SSX2 | 0.001783 |
| FAT3 | 0.001783 |
| SLURP1 | 0.001782 |
| WDSUB1 | 0.001780 |
| MIXL1 | 0.001780 |
| GABPA | 0.001780 |
| ZNF787 | 0.001780 |
| C4orf45 | 0.001780 |
| EIF2AK1 | 0.001779 |
| OR2AT4 | 0.001779 |
| DMRTB1 | 0.001776 |
| IGSF23 | 0.001776 |
| YTHDF1 | 0.001775 |
| LARP1 | 0.001775 |
| ZC3HC1 | 0.001775 |
| OR8D4 | 0.001775 |
| OR4A15 | 0.001775 |
| ZBTB8OS | 0.001774 |
| CCDC117 | 0.001774 |
| GTPBP4 | 0.001774 |
| NDUFS1 | 0.001773 |
| AXIN1 | 0.001772 |
| MEI4 | 0.001772 |
| TRIM64B | 0.001772 |
| RBFOX2 | 0.001771 |
| ADSSL1 | 0.001771 |
| PTK2 | 0.001770 |
| HDAC10 | 0.001769 |
| ARNTL | 0.001768 |
| OR11H4 | 0.001768 |
| ZNF687 | 0.001768 |
| CHRNB3 | 0.001767 |
| PINX1 | 0.001766 |
| ANKRD46 | 0.001765 |
| TTC30A | 0.001765 |
| GOLGA8N | 0.001763 |
| RTN4IP1 | 0.001762 |
| PTPRA | 0.001762 |
| CREB3L3 | 0.001762 |
| PIGZ | 0.001760 |
| PIGF | 0.001760 |
| NME7 | 0.001760 |
| PATL1 | 0.001759 |
| MRPS10 | 0.001759 |
| COX5A | 0.001757 |
| POPDC2 | 0.001757 |
| ENOX2 | 0.001756 |
| ITIH1 | 0.001756 |
| TRIM68 | 0.001755 |
| YARS2 | 0.001755 |
| UBE3A | 0.001754 |
| GPR176 | 0.001754 |
| MRGPRX3 | 0.001754 |
| ELK4 | 0.001754 |
| GPR1 | 0.001752 |
| CBL | 0.001752 |
| TRUB2 | 0.001751 |
| FAM151A | 0.001751 |
| OR7C1 | 0.001748 |
| ZNF600 | 0.001747 |
| XPNPEP3 | 0.001746 |
| OR56A1 | 0.001745 |
| KDM8 | 0.001744 |
| MFAP3 | 0.001743 |
| OR11A1 | 0.001743 |
| MAVS | 0.001742 |
| EPHX4 | 0.001742 |
| RC3H1 | 0.001741 |
| USP29 | 0.001740 |
| ZP3 | 0.001739 |
| ISCA1 | 0.001739 |
| ATXN7L3 | 0.001739 |
| DMRTC2 | 0.001738 |
| EEPD1 | 0.001735 |
| MTHFD1L | 0.001735 |
| MYF6 | 0.001734 |
| SLC6A18 | 0.001734 |
| PTMS | 0.001733 |
| EPRS | 0.001731 |
| C14orf178 | 0.001729 |
| TFB2M | 0.001728 |
| SYTL5 | 0.001728 |
| KRT82 | 0.001728 |
| POM121L2 | 0.001728 |
| NUDT4 | 0.001728 |
| C9orf85 | 0.001728 |
| TRIM49D1 | 0.001728 |
| APTX | 0.001727 |
| KRTAP24-1 | 0.001727 |
| KCTD15 | 0.001725 |
| NSMCE2 | 0.001725 |
| C14orf166 | 0.001725 |
| CNTN4 | 0.001725 |
| PSMG2 | 0.001724 |
| HEXIM2 | 0.001724 |
| OR4D2 | 0.001724 |
| SS18L2 | 0.001723 |
| ZNF415 | 0.001722 |
| BTBD2 | 0.001721 |
| SLC7A4 | 0.001721 |
| CDY2A | 0.001721 |
| PPP6R1 | 0.001718 |
| AGBL3 | 0.001718 |
| SPEM1 | 0.001718 |
| AGO4 | 0.001717 |
| RNF115 | 0.001715 |
| WDR33 | 0.001714 |
| PISD | 0.001714 |
| PUS3 | 0.001713 |
| C8orf33 | 0.001713 |
| CLSTN1 | 0.001712 |
| XPO6 | 0.001712 |
| GEMIN6 | 0.001712 |
| OR1S2 | 0.001711 |
| SERPINB13 | 0.001711 |
| TSSC1 | 0.001710 |
| AMDHD2 | 0.001710 |
| KRAS | 0.001710 |
| NME9 | 0.001710 |
| MORC4 | 0.001710 |
| GTF2E1 | 0.001709 |
| NOL9 | 0.001709 |
| RPL10L | 0.001709 |
| EIF3B | 0.001709 |
| OR8B3 | 0.001707 |
| UPF1 | 0.001707 |
| NOLC1 | 0.001706 |
| PAK2 | 0.001706 |
| ECU | 0.001706 |
| IQCE | 0.001706 |
| CIAO1 | 0.001705 |
| WDR77 | 0.001705 |
| TTC33 | 0.001705 |
| COQ7 | 0.001705 |
| MROH7 | 0.001704 |
| ADM5 | 0.001704 |
| MRPL39 | 0.001704 |
| EFS | 0.001703 |
| MFF | 0.001703 |
| TRIMS1 | 0.001703 |
| TCEA1 | 0.001702 |
| ARIH2 | 0.001702 |
| DDX41 | 0.001702 |
| OSER1 | 0.001701 |
| CES5A | 0.001700 |
| CEP120 | 0.001699 |
| OR51B6 | 0.001699 |
| RNF215 | 0.001698 |
| TATDN3 | 0.001698 |
| PABPC5 | 0.001697 |
| PRDM14 | 0.001697 |
| IDH3B | 0.001697 |
| VPS37A | 0.001697 |
| CALB2 | 0.001696 |
| MRGPRX1 | 0.001696 |
| NPSR1 | 0.001696 |
| KRT86 | 0.001693 |
| PIP4K2B | 0.001693 |
| BTBD7 | 0.001693 |
| OR4K5 | 0.001691 |
| CYP2C19 | 0.001691 |
| TMEM211 | 0.001690 |
| ARHGAP35 | 0.001687 |
| NELFE | 0.001687 |
| APOL5 | 0.001685 |
| R3HCC1L | 0.001685 |
| OR56B1 | 0.001684 |
| GNPAT | 0.001683 |
| S100A1 | 0.001683 |
| DPH1 | 0.001682 |
| TRIM32 | 0.001682 |
| KIAA0355 | 0.001680 |
| AFMID | 0.001680 |
| RNF31 | 0.001680 |
| CHST10 | 0.001680 |
| MTRNR2L5 | 0.001679 |
| JADE1 | 0.001678 |
| MCTS1 | 0.001678 |
| GCG | 0.001678 |
| EFCAB12 | 0.001678 |
| PREP | 0.001678 |
| C1orf185 | 0.001677 |
| ENTPD7 | 0.001676 |
| HERC4 | 0.001676 |
| TTLL12 | 0.001675 |
| EMC8 | 0.001675 |
| FER1L5 | 0.001675 |
| TRIM50 | 0.001675 |
| UBE2D2 | 0.001674 |
| ZNF143 | 0.001673 |
| HEPACAM | 0.001671 |
| SMAP1 | 0.001671 |
| ZSWIM8 | 0.001671 |
| WDR59 | 0.001671 |
| NUDT16L1 | 0.001670 |
| TNNI3K | 0.001670 |
| LEO1 | 0.001669 |
| DDX59 | 0.001667 |
| HIRA | 0.001667 |
| CARD8 | 0.001667 |
| STRN | 0.001666 |
| SYNE3 | 0.001665 |
| SLC35G3 | 0.001665 |
| MRPL35 | 0.001664 |
| CHRNA7 | 0.001664 |
| OR1B1 | 0.001664 |
| SMAD1 | 0.001663 |
| ROCK1 | 0.001663 |
| KIF2B | 0.001661 |
| ERMARD | 0.001661 |
| MTRNR2L13 | 0.001661 |
| SLC17A1 | 0.001660 |
| ARL4C | 0.001660 |
| CNOT4 | 0.001659 |
| HTRA2 | 0.001659 |
| ZC3H15 | 0.001658 |
| ACADM | 0.001656 |
| KRT31 | 0.001656 |
| SLC25A19 | 0.001654 |
| SNTA1 | 0.001654 |
| GRM1 | 0.001654 |
| PC | 0.001653 |
| CCDC50 | 0.001653 |
| BACE1 | 0.001651 |
| TMEM238 | 0.001651 |
| RBMY1D | 0.001651 |
| NIPAL1 | 0.001650 |
| TMOD4 | 0.001650 |
| CLDN25 | 0.001650 |
| CAPZA3 | 0.001650 |
| PHF20 | 0.001649 |
| ODC1 | 0.001649 |
| TXNRD3 | 0.001648 |
| PLAA | 0.001648 |
| BAGS | 0.001648 |
| PTPN3 | 0.001647 |
| ZFAND4 | 0.001647 |
| ATXN2 | 0.001647 |
| ALDH5A1 | 0.001646 |
| SCHIP1 | 0.001644 |
| ADCK1 | 0.001644 |
| CETN1 | 0.001642 |
| LURAP1 | 0.001641 |
| FBXL15 | 0.001641 |
| TRAPPC8 | 0.001640 |
| NHP2 | 0.001640 |
| TRMT2B | 0.001639 |
| EIF5 | 0.001638 |
| DFFA | 0.001638 |
| PRPF31 | 0.001637 |
| ARL14EP | 0.001637 |
| USP17L22 | 0.001636 |
| OR2B3 | 0.001636 |
| RWDD4 | 0.001636 |
| MKRN1 | 0.001636 |
| GSG1 | 0.001635 |
| CCDC43 | 0.001634 |
| BOLA1 | 0.001634 |
| CLIP2 | 0.001633 |
| ISY1 | 0.001632 |
| COQ5 | 0.001631 |
| SLC6A1 | 0.001630 |
| PEX26 | 0.001629 |
| KRTAP20-2 | 0.001629 |
| GP5 | 0.001629 |
| CACNA1C | 0.001629 |
| ANKRD13C | 0.001628 |
| MINPP1 | 0.001628 |
| CLYBL | 0.001628 |
| LCMT2 | 0.001627 |
| MED7 | 0.001627 |
| YPEL2 | 0.001627 |
| AHSA1 | 0.001626 |
| TOM1L1 | 0.001626 |
| NUP37 | 0.001626 |
| ATP6V0B | 0.001624 |
| SRR | 0.001624 |
| MAPRE1 | 0.001624 |
| SNRPD3 | 0.001622 |
| AMZ2 | 0.001622 |
| ZNF479 | 0.001621 |
| TMEM38A | 0.001621 |
| PSMD2 | 0.001621 |
| CHD4 | 0.001621 |
| KAT8 | 0.001620 |
| CCDC120 | 0.001619 |
| CST9 | 0.001619 |
| VPS29 | 0.001619 |
| MBD5 | 0.001618 |
| WDR3 | 0.001618 |
| OR1F1 | 0.001617 |
| PDE3B | 0.001617 |
| GNB1 | 0.001615 |
| C1orf122 | 0.001613 |
| FAM120B | 0.001613 |
| C17orf98 | 0.001613 |
| TEX40 | 0.001613 |
| PPP3R1 | 0.001612 |
| KDM4A | 0.001612 |
| GTF2F2 | 0.001611 |
| PIWIL4 | 0.001610 |
| PSMB7 | 0.001609 |
| GGA3 | 0.001609 |
| NDUFV1 | 0.001608 |
| TMEM131 | 0.001608 |
| FAM43B | 0.001608 |
| MTO1 | 0.001608 |
| SLITRK4 | 0.001607 |
| GNE | 0.001607 |
| HSD17B10 | 0.001607 |
| CCDC33 | 0.001606 |
| TMEM199 | 0.001606 |
| OR4C5 | 0.001606 |
| YES1 | 0.001605 |
| CAPN7 | 0.001604 |
| SSBP1 | 0.001604 |
| EBF1 | 0.001603 |
| VSIG10 | 0.001603 |
| ATP9B | 0.001602 |
| MAN2A2 | 0.001602 |
| GPAM | 0.001602 |
| PIK3R4 | 0.001602 |
| FAM149B1 | 0.001601 |
| TLE3 | 0.001601 |
| OR4K14 | 0.001601 |
| FRAT2 | 0.001600 |
| FERD3L | 0.001600 |
| DOK6 | 0.001600 |
| NPM3 | 0.001600 |
| NPLOC4 | 0.001600 |
| MAPRE3 | 0.001599 |
| SPX | 0.001599 |
| PTPN4 | 0.001599 |
| SDAD1 | 0.001598 |
| HNRNPK | 0.001597 |
| BRAP | 0.001596 |
| B3GALT6 | 0.001596 |
| DEFA6 | 0.001596 |
| TAF9 | 0.001595 |
| MMP10 | 0.001595 |
| CUEDC1 | 0.001595 |
| BBS5 | 0.001595 |
| HSPA9 | 0.001593 |
| NEK3 | 0.001593 |
| AADACL3 | 0.001593 |
| NIF3L1 | 0.001593 |
| VMP1 | 0.001592 |
| ASH2L | 0.001592 |
| OR9G1 | 0.001592 |
| BCO2 | 0.001592 |
| ADAMTS2 | 0.001592 |
| KRTAP9-3 | 0.001591 |
| EIF2B2 | 0.001591 |
| CCDC82 | 0.001591 |
| OR4Q3 | 0.001590 |
| DUSP9 | 0.001590 |
| OR1I1 | 0.001590 |
| NXT1 | 0.001589 |
| PHF1 | 0.001589 |
| TSPAN16 | 0.001588 |
| OR4K15 | 0.001588 |
| C17orf99 | 0.001587 |
| OR52J3 | 0.001587 |
| DHX16 | 0.001586 |
| MRPS16 | 0.001586 |
| TP53I13 | 0.001586 |
| NCOA2 | 0.001586 |
| NUDT17 | 0.001586 |
| OR2M5 | 0.001586 |
| ENDOG | 0.001585 |
| HSD3B1 | 0.001585 |
| FAM189B | 0.001584 |
| TEX37 | 0.001584 |
| CCS | 0.001584 |
| PRRC2C | 0.001582 |
| ZC2HC1C | 0.001580 |
| ZNF214 | 0.001580 |
| ZBED1 | 0.001580 |
| PYCR2 | 0.001579 |
| AMT | 0.001579 |
| ZSCAN32 | 0.001579 |
| OR2T6 | 0.001578 |
| CANT1 | 0.001578 |
| SRCAP | 0.001577 |
| PRSS56 | 0.001577 |
| CEP104 | 0.001574 |
| EDC3 | 0.001574 |
| CCDC125 | 0.001574 |
| EXOC7 | 0.001573 |
| OR1G1 | 0.001573 |
| NSF | 0.001573 |
| C2orf81 | 0.001572 |
| FAM86B1 | 0.001572 |
| ENTPD5 | 0.001572 |
| SPECC1 | 0.001569 |
| RSAD1 | 0.001568 |
| TRIM41 | 0.001567 |
| OR2T4 | 0.001567 |
| TMEM138 | 0.001566 |
| CRISP1 | 0.001566 |
| RRP36 | 0.001565 |
| SEC14L2 | 0.001565 |
| PHC2 | 0.001564 |
| STRADB | 0.001564 |
| PSMC4 | 0.001564 |
| OR6M1 | 0.001563 |
| IQCF1 | 0.001563 |
| NDUFAB1 | 0.001560 |
| RUNDC1 | 0.001560 |
| RNF44 | 0.001560 |
| ZNF879 | 0.001560 |
| DNAJC13 | 0.001560 |
| PPP1R12A | 0.001560 |
| CNBD1 | 0.001558 |
| ZSCAN31 | 0.001558 |
| TRNAU1AP | 0.001558 |
| TMC03 | 0.001558 |
| PIGM | 0.001557 |
| GH2 | 0.001556 |
| IRF7 | 0.001555 |
| RPA1 | 0.001554 |
| TRABD | 0.001554 |
| ALDH1L2 | 0.001554 |
| OR1D5 | 0.001553 |
| GOT1 | 0.001553 |
| SYNJ2BP | 0.001553 |
| GOSR1 | 0.001552 |
| METTL23 | 0.001552 |
| ABL1 | 0.001551 |
| CCDC121 | 0.001551 |
| CORO7 | 0.001551 |
| UBE2W | 0.001551 |
| NELFB | 0.001550 |
| CLSTN3 | 0.001550 |
| CSN3 | 0.001549 |
| OR8K1 | 0.001549 |
| OR6K2 | 0.001549 |
| TTC2IA | 0.001548 |
| KRT38 | 0.001548 |
| BMS1 | 0.001547 |
| HYOU1 | 0.001547 |
| KIF3B | 0.001547 |
| DCLRE1B | 0.001545 |
| MED22 | 0.001545 |
| FBXO21 | 0.001544 |
| MSH3 | 0.001544 |
| SLC5A10 | 0.001544 |
| NRXN3 | 0.001543 |
| MT1F | 0.001543 |
| ZNF624 | 0.001542 |
| RFC1 | 0.001542 |
| ABHD8 | 0.001542 |
| LMLN | 0.001542 |
| EVI5L | 0.001542 |
| C1orf101 | 0.001541 |
| NCR2 | 0.001540 |
| IGF2BP1 | 0.001538 |
| UBE4A | 0.001538 |
| RTP2 | 0.001538 |
| C9orf57 | 0.001538 |
| DR1 | 0.001537 |
| WDR74 | 0.001537 |
| ZFX | 0.001534 |
| ZFYVE16 | 0.001534 |
| SETD1B | 0.001532 |
| KRTAP2-3 | 0.001532 |
| SLC22A24 | 0.001532 |
| ZC2HC1B | 0.001531 |
| OTUD6A | 0.001531 |
| TBL1Y | 0.001531 |
| MCCC1 | 0.001530 |
| CCNC | 0.001530 |
| MPHOSPH10 | 0.001530 |
| KIAA1755 | 0.001530 |
| ASPRV1 | 0.001529 |
| RGS21 | 0.001529 |
| PAKI | 0.001528 |
| OR10H5 | 0.001527 |
| OR5A2 | 0.001526 |
| SLTM | 0.001526 |
| LIPF | 0.001526 |
| CWC15 | 0.001525 |
| NUFIP1 | 0.001524 |
| TRIM49B | 0.001524 |
| POLR3B | 0.001523 |
| GNL1 | 0.001523 |
| IFNA5 | 0.001523 |
| FAM47E | 0.001522 |
| CUL4A | 0.001521 |
| FAM96A | 0.001521 |
| OR6S1 | 0.001521 |
| ANKK1 | 0.001521 |
| ARFGAP3 | 0.001520 |
| C12orf60 | 0.001520 |
| ZNF22 | 0.001519 |
| KBTBD2 | 0.001519 |
| STOML2 | 0.001519 |
| MYOM3 | 0.001519 |
| NYX | 0.001519 |
| OR9Q1 | 0.001518 |
| ZDHHC11B | 0.001518 |
| PREB | 0.001518 |
| FKRP | 0.001517 |
| CNST | 0.001516 |
| PLEKHA7 | 0.001516 |
| GOLGA1 | 0.001516 |
| NPHP3 | 0.001515 |
| MAP3K13 | 0.001515 |
| ROGDI | 0.001515 |
| ZNF264 | 0.001514 |
| CCDC86 | 0.001514 |
| FBXW8 | 0.001514 |
| OR52D1 | 0.001513 |
| KLRC4 | 0.001513 |
| DDB2 | 0.001512 |
| C15orf65 | 0.001512 |
| MNT | 0.001512 |
| OR5V1 | 0.001511 |
| ABL2 | 0.001511 |
| DCTN3 | 0.001511 |
| NMD3 | 0.001508 |
| PA2G4 | 0.001508 |
| OR4B1 | 0.001508 |
| NFKBID | 0.001508 |
| KLHL9 | 0.001506 |
| TRIM60 | 0.001504 |
| BSDC1 | 0.001503 |
| NDUFA5 | 0.001502 |
| OR2T5 | 0.001502 |
| COPS6 | 0.001501 |
| ATP5J2 | 0.001501 |
| RPS6KB2 | 0.001501 |
| MTMR2 | 0.001500 |
| THOC5 | 0.001499 |
| B4GALT3 | 0.001498 |
| CXorf40A | 0.001496 |
| OR4M2 | 0.001496 |
| CDK14 | 0.001495 |
| KRTAP4-7 | 0.001495 |
| CSRP3 | 0.001495 |
| GCNT6 | 0.001495 |
| GNAI3 | 0.001495 |
| LZTS3 | 0.001493 |
| C6orf62 | 0.001493 |
| UPF2 | 0.001492 |
| GCC2 | 0.001491 |
| COMMD2 | 0.001491 |
| 43800 | 0.001490 |
| DNAJC25 | 0.001490 |
| ZCCHC4 | 0.001489 |
| KIAA0556 | 0.001489 |
| POLR2G | 0.001488 |
| SON | 0.001488 |
| ADAT1 | 0.001486 |
| OR2T34 | 0.001486 |
| RNF38 | 0.001485 |
| ACSM2A | 0.001484 |
| SPANXN1 | 0.001484 |
| GFM1 | 0.001483 |
| COX6A1 | 0.001483 |
| GOLGA6L4 | 0.001483 |
| COL25A1 | 0.001482 |
| RCHY1 | 0.001482 |
| KCTD17 | 0.001481 |
| LRRC75B | 0.001481 |
| ZNF174 | 0.001480 |
| AP4E1 | 0.001480 |
| XAGE5 | 0.001480 |
| TMUB1 | 0.001479 |
| NIT1 | 0.001479 |
| CYP8B1 | 0.001478 |
| CCZ1 | 0.001477 |
| IL20 | 0.001476 |
| OR2G2 | 0.001476 |
| PRAMEF14 | 0.001476 |
| ATP5G1 | 0.001476 |
| GIT1 | 0.001476 |
| OR51G1 | 0.001476 |
| OR2AG1 | 0.001475 |
| KDM5A | 0.001475 |
| FAM207A | 0.001475 |
| RASD1 | 0.001475 |
| NRSN2 | 0.001474 |
| OR6T1 | 0.001473 |
| TRUB1 | 0.001472 |
| SPANXN2 | 0.001472 |
| ZNF677 | 0.001472 |
| TBPL2 | 0.001471 |
| SETD2 | 0.001471 |
| TREM77 | 0.001471 |
| UTP14A | 0.001469 |
| CALHM3 | 0.001469 |
| ADSL | 0.001469 |
| KLHL13 | 0.001468 |
| KDF1 | 0.001468 |
| RABEP1 | 0.001468 |
| OR52I2 | 0.001466 |
| CCDC179 | 0.001466 |
| CIZ1 | 0.001466 |
| PPIL4 | 0.001466 |
| FBXL2 | 0.001466 |
| OTOP1 | 0.001465 |
| TRIM49C | 0.001465 |
| SYCE3 | 0.001464 |
| WDR64 | 0.001464 |
| PRPS1L1 | 0.001464 |
| CLDN19 | 0.001464 |
| IRF3 | 0.001463 |
| TMEM186 | 0.001462 |
| FASTKD2 | 0.001462 |
| UBAP2L | 0.001462 |
| CSNK2B | 0.001462 |
| APPBP2 | 0.001462 |
| BOLA2B | 0.001461 |
| TMEM74B | 0.001461 |
| MGAT2 | 0.001461 |
| FBXO24 | 0.001461 |
| TECTB | 0.001460 |
| OR10J3 | 0.001459 |
| SCARB1 | 0.001459 |
| MS4A12 | 0.001458 |
| CHMP4C | 0.001457 |
| FAHD2A | 0.001457 |
| WDR24 | 0.001456 |
| ABCF1 | 0.001455 |
| OR2T12 | 0.001455 |
| JMJD1C | 0.001455 |
| GTF2A2 | 0.001454 |
| ANTXRL | 0.001454 |
| FAM168B | 0.001454 |
| MRPS6 | 0.001453 |
| SPATA24 | 0.001452 |
| PRAMEF15 | 0.001452 |
| COG8 | 0.001452 |
| OR4K2 | 0.001452 |
| UTP15 | 0.001451 |
| KRTAP3-1 | 0.001451 |
| GHRL | 0.001450 |
| SLC38A2 | 0.001449 |
| MAD2L1BP | 0.001449 |
| OR11H6 | 0.001448 |
| MCMBP | 0.001447 |
| ATPIF1 | 0.001447 |
| HS3ST5 | 0.001446 |
| PDS5A | 0.001446 |
| RCOR1 | 0.001446 |
| DUSP10 | 0.001445 |
| ALKBH4 | 0.001444 |
| ZC3H7A | 0.001444 |
| CXorf56 | 0.001443 |
| TRIM43B | 0.001443 |
| TRIP11 | 0.001443 |
| RPIA | 0.001443 |
| OR5D14 | 0.001442 |
| DCAF5 | 0.001441 |
| HNF4G | 0.001441 |
| MS4A13 | 0.001439 |
| POP4 | 0.001439 |
| CETN3 | 0.001438 |
| PSG3 | 0.001438 |
| OR9Q2 | 0.001437 |
| CHCHD3 | 0.001436 |
| ABCF3 | 0.001436 |
| CYB561D1 | 0.001435 |
| ZNRF4 | 0.001434 |
| MRPS11 | 0.001433 |
| KRTAP5-10 | 0.001432 |
| SPIDR | 0.001432 |
| TULP3 | 0.001432 |
| GOLGB1 | 0.001431 |
| KRT84 | 0.001431 |
| OR13C3 | 0.001430 |
| OR52M1 | 0.001429 |
| NDUFB3 | 0.001429 |
| KIAA1109 | 0.001429 |
| IFNA21 | 0.001428 |
| PPP2R3C | 0.001427 |
| NEK1 | 0.001427 |
| SDR42E2 | 0.001425 |
| RBPJ | 0.001425 |
| CCT4 | 0.001424 |
| TRMT12 | 0.001423 |
| OR13C4 | 0.001422 |
| TBCK | 0.001420 |
| MRPL15 | 0.001420 |
| MLLT3 | 0.001420 |
| VPS54 | 0.001420 |
| OR2M2 | 0.001417 |
| ARGFX | 0.001417 |
| ZNF154 | 0.001416 |
| RIMKLB | 0.001416 |
| GAGE12E | 0.001415 |
| ZNF44 | 0.001415 |
| VTI1A | 0.001415 |
| CDKN1B | 0.001414 |
| ZNRF1 | 0.001413 |
| SORL1 | 0.001413 |
| C11orf58 | 0.001413 |
| CTU2 | 0.001411 |
| KDELC2 | 0.001411 |
| POLR1D | 0.001410 |
| IL31 | 0.001410 |
| OR13C8 | 0.001410 |
| PPIG | 0.001410 |
| LHCGR | 0.001409 |
| LRRC47 | 0.001409 |
| SPOPL | 0.001409 |
| C16orf72 | 0.001407 |
| ZNF410 | 0.001406 |
| RSBN1L | 0.001406 |
| PFDN2 | 0.001405 |
| FBXL20 | 0.001405 |
| MUT | 0.001405 |
| LCE1B | 0.001405 |
| STARD4 | 0.001404 |
| SIN3A | 0.001403 |
| TIMM8B | 0.001402 |
| SNIP1 | 0.001402 |
| P3H3 | 0.001401 |
| NR2E3 | 0.001400 |
| ZCCHC6 | 0.001398 |
| ZNF429 | 0.001398 |
| GALT | 0.001397 |
| OR14I1 | 0.001397 |
| MRPL22 | 0.001394 |
| SCRN3 | 0.001391 |
| DYNC1H1 | 0.001391 |
| TMEM239 | 0.001390 |
| IKBKB | 0.001390 |
| GNA13 | 0.001390 |
| IGFBP1 | 0.001389 |
| KEAP1 | 0.001389 |
| PTBP3 | 0.001389 |
| TIMM10 | 0.001387 |
| H1F0 | 0.001387 |
| THTPA | 0.001387 |
| SCYL3 | 0.001386 |
| COG2 | 0.001386 |
| YWHAG | 0.001386 |
| MBTPS2 | 0.001385 |
| DHX30 | 0.001385 |
| PDCL | 0.001385 |
| MACROD1 | 0.001385 |
| POM121C | 0.001384 |
| ZNF189 | 0.001384 |
| RGL4 | 0.001384 |
| ACSM2B | 0.001384 |
| UBQLN1 | 0.001383 |
| MRGPRG | 0.001383 |
| NPHP1 | 0.001382 |
| OR5M11 | 0.001382 |
| USP39 | 0.001382 |
| BOD1L1 | 0.001381 |
| MRPL4 | 0.001381 |
| NDUFB6 | 0.001380 |
| SF1 | 0.001379 |
| WDR60 | 0.001379 |
| GLTSCR1L | 0.001378 |
| SLC41A3 | 0.001378 |
| LRRIQ1 | 0.001378 |
| ELMO2 | 0.001377 |
| SS18 | 0.001377 |
| KIF1BP | 0.001376 |
| TPI1 | 0.001375 |
| YKT6 | 0.001375 |
| TNFAIP8L1 | 0.001375 |
| PELP1 | 0.001374 |
| OR2F2 | 0.001373 |
| OR5M10 | 0.001373 |
| SCD5 | 0.001373 |
| UQCRFS1 | 0.001373 |
| GADD45A | 0.001372 |
| NUDT3 | 0.001371 |
| NAIF1 | 0.001371 |
| SNRNP27 | 0.001370 |
| OR10W1 | 0.001370 |
| ARSH | 0.001370 |
| CEBPZ | 0.001370 |
| TSPAN17 | 0.001369 |
| DNAH17 | 0.001369 |
| LYZL2 | 0.001367 |
| FRMD8 | 0.001367 |
| TGS1 | 0.001367 |
| IFNL2 | 0.001367 |
| CDY2B | 0.001366 |
| FARP1 | 0.001366 |
| EIF2A | 0.001365 |
| MRM1 | 0.001365 |
| YWHAZ | 0.001364 |
| OR2A4 | 0.001364 |
| LMAN1 | 0.001364 |
| DDX6 | 0.001363 |
| COMMD4 | 0.001362 |
| FAM78B | 0.001362 |
| KRTAP19-3 | 0.001361 |
| CISD1 | 0.001361 |
| ZNF652 | 0.001360 |
| PRR12 | 0.001360 |
| TBX6 | 0.001360 |
| ACOT8 | 0.001360 |
| WDR11 | 0.001360 |
| WDR13 | 0.001359 |
| MOG | 0.001359 |
| C21orf140 | 0.001359 |
| CSMD1 | 0.001359 |
| KLK1 | 0.001358 |
| SHPK | 0.001358 |
| OR7A17 | 0.001357 |
| HARBI1 | 0.001357 |
| GGACT | 0.001357 |
| TUBB4A | 0.001356 |
| PRKAG3 | 0.001355 |
| POTED | 0.001355 |
| PPM1G | 0.001355 |
| TAAR2 | 0.001355 |
| OR2T29 | 0.001355 |
| ARFIP2 | 0.001355 |
| GUCA1C | 0.001354 |
| BEND6 | 0.001353 |
| PRAMEF12 | 0.001353 |
| OR2J1 | 0.001353 |
| KRT34 | 0.001352 |
| PSMD3 | 0.001352 |
| MYO9B | 0.001352 |
| C1orf27 | 0.001352 |
| ZNF395 | 0.001352 |
| WBSCR22 | 0.001351 |
| PPP1R12C | 0.001351 |
| GAGE10 | 0.001351 |
| KIAA1328 | 0.001350 |
| IFNA10 | 0.001349 |
| EDEM3 | 0.001348 |
| MICU2 | 0.001348 |
| AASDH | 0.001348 |
| RMND5B | 0.001348 |
| EIF2S3 | 0.001347 |
| TRAPPC11 | 0.001346 |
| OR11H2 | 0.001346 |
| FAM170A | 0.001346 |
| MTRNR2L7 | 0.001345 |
| GMFB | 0.001345 |
| PRR23C | 0.001345 |
| ZC3H12C | 0.001345 |
| OR4D10 | 0.001344 |
| THY1 | 0.001344 |
| WSB1 | 0.001343 |
| RTKN | 0.001343 |
| RNF19A | 0.001342 |
| KRTAP21-1 | 0.001342 |
| PRAMEF8 | 0.001342 |
| PDE6H | 0.001342 |
| OR5AN1 | 0.001341 |
| PRR14 | 0.001341 |
| IFNW1 | 0.001340 |
| C16orf52 | 0.001339 |
| PSMD11 | 0.001339 |
| NOP16 | 0.001339 |
| KIAA1211L | 0.001339 |
| MKL2 | 0.001339 |
| IGFBP3 | 0.001338 |
| SLCO1B7 | 0.001338 |
| FOXR1 | 0.001337 |
| ABHD12 | 0.001337 |
| OR6C1 | 0.001337 |
| HAUS2 | 0.001336 |
| DPP3 | 0.001336 |
| LCAT | 0.001335 |
| CHIC2 | 0.001334 |
| KXD1 | 0.001334 |
| IKZF5 | 0.001333 |
| USP15 | 0.001332 |
| OR2T1 | 0.001332 |
| MRGPRX4 | 0.001330 |
| TM7SF2 | 0.001330 |
| PNPLA1 | 0.001329 |
| LRP6 | 0.001328 |
| PFKL | 0.001328 |
| NDUFA9 | 0.001327 |
| VPS33B | 0.001327 |
| PAGE5 | 0.001326 |
| ZNF746 | 0.001326 |
| TSEN34 | 0.001325 |
| CCNK | 0.001325 |
| NARS | 0.001324 |
| PPIA | 0.001324 |
| AFF4 | 0.001323 |
| HEATR5B | 0.001323 |
| IZUM03 | 0.001322 |
| RANBP10 | 0.001321 |
| SMNDC1 | 0.001320 |
| RBM34 | 0.001320 |
| POMGNT1 | 0.001319 |
| USF2 | 0.001319 |
| FOXR2 | 0.001319 |
| ZC3H18 | 0.001318 |
| EIF4E | 0.001317 |
| ODF4 | 0.001316 |
| OR12D3 | 0.001315 |
| MDM2 | 0.001314 |
| AP4S1 | 0.001314 |
| REG1B | 0.001314 |
| MIEN1 | 0.001313 |
| NPVF | 0.001313 |
| NECAP1 | 0.001313 |
| SLC22A8 | 0.001312 |
| FAM208A | 0.001312 |
| OR13F1 | 0.001312 |
| WDR48 | 0.001312 |
| TTC17 | 0.001311 |
| PIWIL3 | 0.001311 |
| GOLGA3 | 0.001310 |
| OR6C76 | 0.001309 |
| SLC12A7 | 0.001309 |
| NEK9 | 0.001307 |
| GTSF1L | 0.001307 |
| OR14C36 | 0.001306 |
| CCT8L2 | 0.001306 |
| ZNF574 | 0.001306 |
| C1orf68 | 0.001306 |
| PAK4 | 0.001305 |
| STAMBP | 0.001305 |
| ZNF219 | 0.001304 |
| GPBP1L1 | 0.001304 |
| TBC1D23 | 0.001303 |
| SCFD1 | 0.001302 |
| TCTN1 | 0.001301 |
| OR52B6 | 0.001301 |
| LALBA | 0.001300 |
| MLXIP | 0.001300 |
| TRMT10C | 0.001300 |
| ARMC5 | 0.001300 |
| PLEKHA3 | 0.001300 |
| RGPD1 | 0.001298 |
| BBS2 | 0.001298 |
| OR6B1 | 0.001297 |
| PXDNL | 0.001297 |
| WFDC8 | 0.001295 |
| C6orf15 | 0.001295 |
| KRTAP9-4 | 0.001294 |
| FRS2 | 0.001294 |
| TMEM8B | 0.001293 |
| DDT | 0.001293 |
| GPR161 | 0.001293 |
| EIF2S1 | 0.001293 |
| SLC39A9 | 0.001292 |
| ZG16 | 0.001291 |
| MAN1A2 | 0.001291 |
| TRIM25 | 0.001290 |
| COG5 | 0.001289 |
| SAP130 | 0.001289 |
| WDFY2 | 0.001287 |
| CDY1 | 0.001287 |
| GFRAL | 0.001286 |
| PCCA | 0.001286 |
| OR4K17 | 0.001286 |
| OR4F6 | 0.001286 |
| PRCC | 0.001286 |
| GNAS | 0.001285 |
| METAP1 | 0.001285 |
| RNF121 | 0.001284 |
| KRT36 | 0.001283 |
| CLCA1 | 0.001283 |
| ZNF502 | 0.001282 |
| NLRP4 | 0.001282 |
| VN1R4 | 0.001281 |
| RPL38 | 0.001281 |
| OR4N5 | 0.001281 |
| SDS | 0.001280 |
| PRR30 | 0.001279 |
| MROH5 | 0.001279 |
| OR4D1 | 0.001278 |
| OR52K1 | 0.001278 |
| OR5M8 | 0.001277 |
| MIIP | 0.001277 |
| RPGRIP1 | 0.001276 |
| GTF3A | 0.001275 |
| CDKL3 | 0.001274 |
| PBX1 | 0.001273 |
| ANP32B | 0.001273 |
| TFB1M | 0.001273 |
| ANKMY1 | 0.001273 |
| KRTAP26-1 | 0.001271 |
| ATPAF2 | 0.001271 |
| OR2W1 | 0.001270 |
| PNLIP | 0.001269 |
| ATP5J | 0.001269 |
| HMGXB3 | 0.001268 |
| OR4N4 | 0.001268 |
| BAZ2A | 0.001267 |
| PIGG | 0.001264 |
| ARID4A | 0.001264 |
| PIBF1 | 0.001262 |
| GEMIN7 | 0.001262 |
| DNAJC7 | 0.001261 |
| CUL3 | 0.001259 |
| HS6ST1 | 0.001259 |
| NACC1 | 0.001258 |
| SCCPDH | 0.001258 |
| COL1A1 | 0.001258 |
| TCEAL3 | 0.001257 |
| TXNL1 | 0.001257 |
| PYCRL | 0.001256 |
| FAM102A | 0.001256 |
| CNP | 0.001256 |
| OR10K2 | 0.001255 |
| HELQ | 0.001255 |
| GOSR2 | 0.001252 |
| KCNV1 | 0.001251 |
| PRAMEF26 | 0.001251 |
| MRPL12 | 0.001251 |
| OR2G6 | 0.001250 |
| KRTAP13-2 | 0.001250 |
| LIPT1 | 0.001250 |
| SAP30BP | 0.001249 |
| TMA16 | 0.001249 |
| CDK5 | 0.001248 |
| RNF152 | 0.001248 |
| POLR1E | 0.001248 |
| CPNE1 | 0.001247 |
| OR6K6 | 0.001247 |
| OR2A42 | 0.001247 |
| CNTROB | 0.001247 |
| RAB28 | 0.001247 |
| GMPPB | 0.001247 |
| TDO2 | 0.001246 |
| UBN1 | 0.001246 |
| KIAA1683 | 0.001246 |
| KRT35 | 0.001246 |
| DMGDH | 0.001246 |
| OR6C75 | 0.001245 |
| CHD9 | 0.001245 |
| OR5M9 | 0.001245 |
| CDC23 | 0.001245 |
| PDZD11 | 0.001245 |
| GJA10 | 0.001245 |
| TTI2 | 0.001244 |
| IQCD | 0.001243 |
| CYP19A1 | 0.001243 |
| MTFR1L | 0.001240 |
| HIRIP3 | 0.001239 |
| C5orf58 | 0.001239 |
| COA4 | 0.001238 |
| PSMD10 | 0.001238 |
| SMG8 | 0.001238 |
| MAPRE2 | 0.001237 |
| KCNK18 | 0.001236 |
| BHLHA9 | 0.001236 |
| ALDH18A1 | 0.001235 |
| OR52E2 | 0.001235 |
| THEM6 | 0.001235 |
| IFIT1B | 0.001235 |
| FRG2B | 0.001235 |
| ELP4 | 0.001234 |
| XPR1 | 0.001233 |
| UQCRB | 0.001233 |
| CHUK | 0.001233 |
| RESP18 | 0.001231 |
| HIST1H2AA | 0.001231 |
| RDII8 | 0.001231 |
| MAPIA | 0.001230 |
| THRAP3 | 0.001230 |
| PNPLA8 | 0.001229 |
| HGH1 | 0.001229 |
| SOD1 | 0.001227 |
| PSMB2 | 0.001227 |
| TMPRSS12 | 0.001227 |
| TMEM95 | 0.001227 |
| TAF10 | 0.001226 |
| TTC30B | 0.001226 |
| SF3B6 | 0.001225 |
| ZDHHC6 | 0.001225 |
| KRTAP21-2 | 0.001225 |
| HCN1 | 0.001223 |
| SEBOX | 0.001222 |
| DEFB125 | 0.001222 |
| EBNA1BP2 | 0.001221 |
| GMEB1 | 0.001220 |
| RPTN | 0.001220 |
| GTF2H3 | 0.001218 |
| SLC25A31 | 0.001217 |
| OR5B21 | 0.001217 |
| ARHGEF11 | 0.001216 |
| GJB4 | 0.001216 |
| TBC1D10B | 0.001216 |
| MRPL11 | 0.001215 |
| MT-ATP8 | 0.001213 |
| GHSR | 0.001213 |
| FAM213B | 0.001213 |
| RNF133 | 0.001213 |
| PLET1 | 0.001212 |
| TRPV6 | 0.001212 |
| MRPL32 | 0.001211 |
| SYTL4 | 0.001210 |
| OR10T2 | 0.001210 |
| TTC7B | 0.001209 |
| GJD3 | 0.001208 |
| ARHGEF9 | 0.001208 |
| NAALAD2 | 0.001207 |
| KRTAP9-7 | 0.001207 |
| B3GALT2 | 0.001206 |
| TSR1 | 0.001206 |
| LCMT1 | 0.001205 |
| ZZEF1 | 0.001205 |
| KRTAP9-8 | 0.001205 |
| RAB23 | 0.001203 |
| OR5A1 | 0.001203 |
| ARMCX2 | 0.001203 |
| UBQLN2 | 0.001203 |
| OR5K4 | 0.001202 |
| TFAP2E | 0.001202 |
| ZFYVE19 | 0.001201 |
| EARS2 | 0.001201 |
| THAP1 | 0.001201 |
| EMC1 | 0.001201 |
| OR4D6 | 0.001200 |
| ZNF257 | 0.001200 |
| CH1C1 | 0.001200 |
| PRDX4 | 0.001198 |
| SLC25A18 | 0.001198 |
| PSMA3 | 0.001197 |
| PRKD3 | 0.001196 |
| TMEM117 | 0.001196 |
| CYLC1 | 0.001196 |
| NDUFV3 | 0.001196 |
| OXNAD1 | 0.001194 |
| C1orf146 | 0.001193 |
| C21orf62 | 0.001193 |
| MCRS1 | 0.001192 |
| ZC2HC1A | 0.001192 |
| ABTB1 | 0.001191 |
| INO80B | 0.001190 |
| TM2D1 | 0.001190 |
| MEIS3 | 0.001190 |
| SURF6 | 0.001189 |
| BAZ1A | 0.001189 |
| FTSJ1 | 0.001188 |
| OR5AU1 | 0.001188 |
| OR8K5 | 0.001188 |
| OR511 | 0.001187 |
| ENKD1 | 0.001186 |
| TBATA | 0.001186 |
| MT-CO2 | 0.001186 |
| GSS | 0.001185 |
| PDCD7 | 0.001185 |
| VPS13C | 0.001185 |
| PPIL3 | 0.001185 |
| OR4K3 | 0.001184 |
| OR1A2 | 0.001184 |
| PPP4R2 | 0.001184 |
| RNF2 | 0.001182 |
| AARS | 0.001181 |
| NET1 | 0.001181 |
| SSX4 | 0.001181 |
| CXorf66 | 0.001181 |
| MRPS18C | 0.001181 |
| LGALS13 | 0.001180 |
| OR6C4 | 0.001179 |
| GGA1 | 0.001179 |
| KRT85 | 0.001179 |
| KTI12 | 0.001179 |
| ATP2A2 | 0.001179 |
| ARHGAP5 | 0.001178 |
| KRTAP7-1 | 0.001178 |
| KRTAP12-3 | 0.001177 |
| KRTAP2-4 | 0.001176 |
| FAM49B | 0.001176 |
| CDK11B | 0.001176 |
| OR10H3 | 0.001175 |
| HAMP | 0.001175 |
| SFT2D2 | 0.001174 |
| KRT12 | 0.001174 |
| VARS | 0.001173 |
| ISCA2 | 0.001173 |
| ZBTB9 | 0.001172 |
| GSTT2B | 0.001171 |
| OR10AG1 | 0.001171 |
| UBE3C | 0.001171 |
| HTR3B | 0.001170 |
| TAS2R41 | 0.001170 |
| TMED4 | 0.001170 |
| OR4C12 | 0.001170 |
| TAS2R16 | 0.001169 |
| WDR43 | 0.001169 |
| OR4C15 | 0.001169 |
| CCNG1 | 0.001167 |
| DAZ3 | 0.001164 |
| STX18 | 0.001164 |
| OR2T3 | 0.001164 |
| SMYD4 | 0.001163 |
| GARS | 0.001163 |
| GOLGA7 | 0.001162 |
| KIF26A | 0.001162 |
| KRTAP2-1 | 0.001162 |
| OR52A1 | 0.001161 |
| OR5K3 | 0.001161 |
| NANOG | 0.001161 |
| ARF5 | 0.001161 |
| SARNP | 0.001161 |
| INO80C | 0.001161 |
| CEACAM19 | 0.001160 |
| TMEM247 | 0.001160 |
| HMBS | 0.001160 |
| GLO1 | 0.001158 |
| TRMT44 | 0.001158 |
| KIAA1143 | 0.001158 |
| PIGN | 0.001157 |
| RAD54L2 | 0.001157 |
| OR7G2 | 0.001157 |
| OR6C74 | 0.001157 |
| MB21D1 | 0.001156 |
| MAGEB16 | 0.001155 |
| POLR1A | 0.001155 |
| SSX3 | 0.001155 |
| NIFK | 0.001155 |
| RD3L | 0.001154 |
| OR4D11 | 0.001154 |
| TRAF7 | 0.001153 |
| OR10A7 | 0.001153 |
| OR10J1 | 0.001153 |
| HIST1H2BK | 0.001153 |
| NOL7 | 0.001152 |
| HNRNPUL2 | 0.001151 |
| RABEP2 | 0.001150 |
| IFT22 | 0.001149 |
| BBS4 | 0.001149 |
| BABAM1 | 0.001149 |
| C1orf56 | 0.001149 |
| SDHA | 0.001148 |
| KPNA6 | 0.001146 |
| TSFM | 0.001146 |
| MT1G | 0.001145 |
| UAP1 | 0.001145 |
| NTMT1 | 0.001144 |
| STAT2 | 0.001142 |
| OR6Y1 | 0.001141 |
| OR1411 | 0.001141 |
| IFNA17 | 0.001141 |
| OR2S2 | 0.001141 |
| TSEN15 | 0.001141 |
| BIRC8 | 0.001140 |
| KIAA0368 | 0.001140 |
| SESTD1 | 0.001140 |
| PDAP1 | 0.001140 |
| OR1P1 | 0.001140 |
| SNX22 | 0.001139 |
| POM121L12 | 0.001139 |
| KRTAP9-9 | 0.001139 |
| OR10H4 | 0.001138 |
| MAPKBP1 | 0.001138 |
| OR4Q2 | 0.001138 |
| SHC4 | 0.001137 |
| VRK3 | 0.001137 |
| ITFG2 | 0.001136 |
| OR5K1 | 0.001135 |
| MRPL2 | 0.001135 |
| GPR162 | 0.001134 |
| ZFAND2B | 0.001133 |
| OR12D1 | 0.001133 |
| MPHOSPH6 | 0.001133 |
| SLC35B2 | 0.001131 |
| MRPL38 | 0.001131 |
| ANKRD65 | 0.001131 |
| EIF2B3 | 0.001131 |
| TPBG | 0.001130 |
| HERC1 | 0.001130 |
| IL22RA1 | 0.001130 |
| CDC20B | 0.001129 |
| YIF1B | 0.001129 |
| NLRP10 | 0.001129 |
| DNAJC16 | 0.001128 |
| SULT6B1 | 0.001128 |
| KRT26 | 0.001127 |
| BUD31 | 0.001127 |
| ARFRP1 | 0.001127 |
| DEFB118 | 0.001126 |
| TWF1 | 0.001126 |
| OR5L2 | 0.001126 |
| OR10G4 | 0.001124 |
| C4orf17 | 0.001124 |
| DGUOK | 0.001124 |
| DEFB105A | 0.001123 |
| APOM | 0.001123 |
| ZNF35 | 0.001123 |
| VCPIP1 | 0.001122 |
| MRPL18 | 0.001122 |
| TBC1D3 | 0.001122 |
| UBTFL1 | 0.001122 |
| IZUMO1R | 0.001121 |
| PERP | 0.001119 |
| PRAMEF2 | 0.001119 |
| NAPEPLD | 0.001119 |
| TRIM10 | 0.001119 |
| PAIP2 | 0.001118 |
| LIX1 | 0.001118 |
| RRNAD1 | 0.001117 |
| TMEM251 | 0.001117 |
| ZNF672 | 0.001117 |
| TECPR1 | 0.001116 |
| SPATA31A7 | 0.001116 |
| BCORL1 | 0.001115 |
| GPATCH11 | 0.001115 |
| NHLRC1 | 0.001114 |
| OR52E8 | 0.001114 |
| KRTAP13-1 | 0.001113 |
| POLR2J | 0.001113 |
| UBE2R2 | 0.001113 |
| PRAMEF7 | 0.001112 |
| GRHL1 | 0.001112 |
| OR4A8 | 0.001111 |
| OR8D2 | 0.001109 |
| OR2C1 | 0.001108 |
| TFPT | 0.001108 |
| ZSWIM4 | 0.001107 |
| GLOD4 | 0.001107 |
| DCPS | 0.001106 |
| OR9I1 | 0.001106 |
| WBP1 | 0.001106 |
| COA6 | 0.001105 |
| ANKDD1A | 0.001105 |
| OR7D4 | 0.001105 |
| FOXN2 | 0.001104 |
| CSHL1 | 0.001104 |
| OR5B2 | 0.001104 |
| KLRC3 | 0.001103 |
| SYCP1 | 0.001103 |
| DDX50 | 0.001103 |
| OR6X1 | 0.001101 |
| VLDLR | 0.001101 |
| PCGF2 | 0.001101 |
| DVL1 | 0.001101 |
| BACH2 | 0.001101 |
| RSPRY1 | 0.001101 |
| OR8H1 | 0.001099 |
| TPRN | 0.001098 |
| ATP1B4 | 0.001098 |
| IQCF3 | 0.001098 |
| PSMB1 | 0.001098 |
| KLHL36 | 0.001097 |
| NRAS | 0.001097 |
| C10orf120 | 0.001097 |
| DENND6B | 0.001097 |
| COL3A1 | 0.001096 |
| ERI1 | 0.001095 |
| ZC3H4 | 0.001094 |
| ZFAND2A | 0.001094 |
| KATNB1 | 0.001094 |
| MAPK1IP1L | 0.001093 |
| SIGMAR1 | 0.001093 |
| KRTAP5-11 | 0.001093 |
| OR5AC1 | 0.001092 |
| EIF1AD | 0.001091 |
| ITCH | 0.001091 |
| AUH | 0.001090 |
| TMEM70 | 0.001090 |
| C22orf29 | 0.001089 |
| BHMG1 | 0.001089 |
| ACTR8 | 0.001088 |
| ORAI2 | 0.001088 |
| CUL2 | 0.001088 |
| RING1 | 0.001087 |
| ENSA | 0.001086 |
| LRP2BP | 0.001085 |
| PARP16 | 0.001084 |
| ZNF705B | 0.001084 |
| AP3M1 | 0.001083 |
| PSMD12 | 0.001083 |
| FGFR1OP2 | 0.001083 |
| CEP170B | 0.001083 |
| OR1D2 | 0.001082 |
| GTF3C2 | 0.001081 |
| CDYL | 0.001080 |
| DLGAP4 | 0.001079 |
| DGCR14 | 0.001079 |
| APIP | 0.001079 |
| NUDT2 | 0.001079 |
| GJB7 | 0.001078 |
| C7orf25 | 0.001077 |
| GAGE12H | 0.001076 |
| MRO | 0.001076 |
| CASP6 | 0.001075 |
| OR6C3 | 0.001075 |
| EIF1AX | 0.001074 |
| OR5H2 | 0.001073 |
| ZSWIM6 | 0.001073 |
| NISCH | 0.001072 |
| TTC4 | 0.001071 |
| CCDC172 | 0.001071 |
| OR2T2 | 0.001070 |
| SENP3 | 0.001070 |
| NXF5 | 0.001070 |
| DYNLT3 | 0.001069 |
| MARK4 | 0.001067 |
| TTC19 | 0.001066 |
| FUT8 | 0.001066 |
| ELP6 | 0.001066 |
| DNA1C14 | 0.001066 |
| TAS1R2 | 0.001065 |
| DEFB126 | 0.001063 |
| DNA1C5 | 0.001062 |
| OR51I2 | 0.001062 |
| DCTN5 | 0.001062 |
| TACO1 | 0.001061 |
| BCL11B | 0.001061 |
| ANP32A | 0.001061 |
| LRFN3 | 0.001061 |
| DYNC2H1 | 0.001061 |
| SLC35E3 | 0.001060 |
| ZNHIT6 | 0.001060 |
| CDKN2AIPNL | 0.001060 |
| DEFAS | 0.001059 |
| OOSP2 | 0.001059 |
| CLEC2A | 0.001058 |
| MID1 | 0.001057 |
| CIB3 | 0.001057 |
| GAS8 | 0.001057 |
| SLC35D1 | 0.001056 |
| OR10J4 | 0.001056 |
| OR10R2 | 0.001056 |
| CEBPZOS | 0.001056 |
| OR5W2 | 0.001055 |
| SIRT2 | 0.001054 |
| MIER2 | 0.001054 |
| EIF4ENIF1 | 0.001052 |
| LELP1 | 0.001052 |
| PRPF18 | 0.001052 |
| MED24 | 0.001051 |
| GOT2 | 0.001050 |
| FNTB | 0.001049 |
| CRLF3 | 0.001048 |
| RBMY1B | 0.001047 |
| AMD1 | 0.001047 |
| VN1R3 | 0.001046 |
| CAMKK2 | 0.001046 |
| TERF2IP | 0.001045 |
| PRDM4 | 0.001045 |
| CLUH | 0.001044 |
| TNP1 | 0.001043 |
| STK38L | 0.001043 |
| LACTBL1 | 0.001043 |
| ATP6V0A1 | 0.001043 |
| EMC10 | 0.001041 |
| FAM222B | 0.001041 |
| CS | 0.001041 |
| OR6J1 | 0.001040 |
| PTPN11 | 0.001040 |
| SURF2 | 0.001040 |
| STK26 | 0.001039 |
| RNF216 | 0.001039 |
| USP47 | 0.001038 |
| OR6C65 | 0.001037 |
| MAGEB18 | 0.001036 |
| LMTK2 | 0.001036 |
| NDUFS7 | 0.001035 |
| C2orf42 | 0.001034 |
| NPM1 | 0.001033 |
| NKX1-1 | 0.001032 |
| DPH6 | 0.001030 |
| CHST13 | 0.001030 |
| NFS1 | 0.001030 |
| FGF8 | 0.001029 |
| RTCA | 0.001028 |
| AP5S1 | 0.001028 |
| NAP1L5 | 0.001027 |
| MS4A10 | 0.001026 |
| OR10J5 | 0.001026 |
| ANAPC15 | 0.001025 |
| ZBTB32 | 0.001025 |
| CES3 | 0.001024 |
| MGAT4A | 0.001024 |
| MBD3L3 | 0.001023 |
| SLC16A7 | 0.001023 |
| B3GNT5 | 0.001022 |
| PPP6R2 | 0.001021 |
| C2CD4B | 0.001020 |
| KIAA0100 | 0.001020 |
| SNX13 | 0.001020 |
| OR2G3 | 0.001020 |
| VEGFA | 0.001020 |
| OR4S1 | 0.001019 |
| STPG1 | 0.001019 |
| SPECC1L | 0.001019 |
| OR51A2 | 0.001019 |
| YIPF4 | 0.001019 |
| C9orf84 | 0.001018 |
| TRAPPC12 | 0.001018 |
| FAMS3C | 0.001017 |
| KIF5B | 0.001017 |
| HDHD3 | 0.001017 |
| G3BP1 | 0.001016 |
| CLCN7 | 0.001016 |
| AKIRIN1 | 0.001016 |
| MRPL19 | 0.001015 |
| NCK2 | 0.001013 |
| DIS3L2 | 0.001013 |
| OR1E3 | 0.001013 |
| ADAMTS12 | 0.001012 |
| RNF5 | 0.001011 |
| LDHA | 0.001011 |
| PRSS54 | 0.001010 |
| DPCD | 0.001010 |
| USMG5 | 0.001009 |
| NGF | 0.001009 |
| CNTNAP1 | 0.001009 |
| OR5M3 | 0.001008 |
| ZNF268 | 0.001005 |
| SCAF11 | 0.001005 |
| MRPS31 | 0.001003 |
| HMGA2 | 0.001003 |
| PCBP2 | 0.001003 |
| IMMT | 0.001003 |
| CD160 | 0.001002 |
| IL21 | 0.001001 |
| RFFL | 0.001000 |
| DNAAF5 | 0.001000 |
| OR5B3 | 0.001000 |
| HDAC3 | 0.000999 |
| SEL1L2 | 0.000999 |
| LIG4 | 0.000997 |
| OR4F5 | 0.000997 |
| TPD52L2 | 0.000996 |
| JKAMP | 0.000995 |
| RBM22 | 0.000995 |
| MTCH2 | 0.000995 |
| PLEKHM3 | 0.000995 |
| LETM1 | 0.000995 |
| COL18A1 | 0.000994 |
| RPL36 | 0.000994 |
| GOLT1B | 0.000994 |
| DPF2 | 0.000993 |
| ATRN | 0.000993 |
| OR52E1 | 0.000993 |
| RHOT1 | 0.000992 |
| DCTN2 | 0.000992 |
| ATF6B | 0.000992 |
| RTN4R | 0.000992 |
| CLDN34 | 0.000991 |
| SMIM7 | 0.000991 |
| CUL7 | 0.000991 |
| OR8J3 | 0.000990 |
| SRL | 0.000990 |
| TMEM174 | 0.000989 |
| ZHX1 | 0.000989 |
| TBRG1 | 0.000989 |
| NUP93 | 0.000988 |
| PIK3C2A | 0.000988 |
| KRT73 | 0.000988 |
| CBX6 | 0.000988 |
| OR1E2 | 0.000988 |
| SLC36A3 | 0.000987 |
| COG4 | 0.000987 |
| OR10H2 | 0.000987 |
| GEMIN5 | 0.000986 |
| CRELD2 | 0.000986 |
| OR4L1 | 0.000986 |
| SRGAP2B | 0.000985 |
| NR1D1 | 0.000985 |
| OR4E2 | 0.000984 |
| DCAF11 | 0.000984 |
| LIAS | 0.000984 |
| NMRAL1 | 0.000983 |
| DUSP8 | 0.000983 |
| DIRC1 | 0.000982 |
| OR6N2 | 0.000982 |
| ZC3H10 | 0.000981 |
| LDB1 | 0.000981 |
| SLC30A6 | 0.000981 |
| MROH2B | 0.000981 |
| OR4K13 | 0.000980 |
| NTHL1 | 0.000980 |
| PRAMEF18 | 0.000979 |
| C21orf91 | 0.000978 |
| GTF2A1L | 0.000978 |
| TAAR5 | 0.000978 |
| KIAA0232 | 0.000977 |
| HNRNPF | 0.000977 |
| DPPA2 | 0.000977 |
| HYKK | 0.000976 |
| THUMPD3 | 0.000976 |
| PCNP | 0.000974 |
| INTS10 | 0.000974 |
| ASXL2 | 0.000974 |
| RDH11 | 0.000973 |
| OR4C45 | 0.000973 |
| CCDC105 | 0.000972 |
| SH3GL1 | 0.000972 |
| WDR45 | 0.000971 |
| MRPL34 | 0.000971 |
| WASF3 | 0.000971 |
| MANF | 0.000970 |
| CNNM3 | 0.000969 |
| ATP5B | 0.000969 |
| HAX1 | 0.000969 |
| TMEM99 | 0.000968 |
| CSN1S1 | 0.000968 |
| OR10C1 | 0.000967 |
| SMCP | 0.000967 |
| FAM9A | 0.000966 |
| LSM12 | 0.000965 |
| MRPL46 | 0.000964 |
| BOLA2 | 0.000963 |
| LRRTM1 | 0.000962 |
| HCG22 | 0.000962 |
| DRG2 | 0.000961 |
| NFYC | 0.000961 |
| EIF2AK3 | 0.000960 |
| OR4F15 | 0.000960 |
| YBX1 | 0.000959 |
| ASB10 | 0.000957 |
| BLOC1S6 | 0.000957 |
| YY1AP1 | 0.000957 |
| OR52N5 | 0.000956 |
| TEX43 | 0.000955 |
| LGALS16 | 0.000955 |
| NUDT14 | 0.000954 |
| LRRN3 | 0.000954 |
| ALG11 | 0.000953 |
| OR6P1 | 0.000952 |
| MLEC | 0.000952 |
| IRF2BP2 | 0.000952 |
| TEP1 | 0.000951 |
| DBP | 0.000951 |
| OR52Z1 | 0.000951 |
| SLC25A32 | 0.000951 |
| ANKRD27 | 0.000950 |
| CWC27 | 0.000950 |
| LYPLA1 | 0.000950 |
| RUNDC3B | 0.000950 |
| RNASE8 | 0.000949 |
| KRTAP4-11 | 0.000949 |
| RAB33B | 0.000949 |
| TGFBR1 | 0.000948 |
| OR5D18 | 0.000948 |
| SSX4B | 0.000948 |
| TAF7 | 0.000947 |
| OR4A5 | 0.000947 |
| GTF3C6 | 0.000946 |
| ARHGAP22 | 0.000946 |
| KRTAP13-3 | 0.000945 |
| VOPP1 | 0.000945 |
| PSG2 | 0.000944 |
| PHLDB1 | 0.000944 |
| APPL1 | 0.000944 |
| KIAA0408 | 0.000944 |
| MAP1LC3A | 0.000944 |
| MCAT | 0.000943 |
| LENG1 | 0.000943 |
| MS4A4E | 0.000942 |
| KCNC3 | 0.000941 |
| CNGA1 | 0.000941 |
| TAF5L | 0.000941 |
| PWP1 | 0.000940 |
| ARMCX3 | 0.000940 |
| NAT10 | 0.000940 |
| CACNG8 | 0.000940 |
| GRSF1 | 0.000939 |
| VPREB1 | 0.000939 |
| RPGR | 0.000938 |
| PPP1R26 | 0.000938 |
| GPC1 | 0.000937 |
| DARS | 0.000937 |
| AK2 | 0.000935 |
| FAM47B | 0.000935 |
| IPO11 | 0.000934 |
| TMEM261 | 0.000934 |
| RFK | 0.000933 |
| AES | 0.000932 |
| CASC10 | 0.000930 |
| LCN9 | 0.000929 |
| CMC1 | 0.000929 |
| PTER | 0.000929 |
| CTBP1 | 0.000928 |
| PRDX3 | 0.000928 |
| CDC73 | 0.000927 |
| OR3A3 | 0.000926 |
| LCE6A | 0.000926 |
| COX6A2 | 0.000925 |
| SWI5 | 0.000925 |
| ZNF629 | 0.000925 |
| MYF5 | 0.000924 |
| FAM192A | 0.000924 |
| NOSIP | 0.000924 |
| CNOT1 | 0.000924 |
| RTP3 | 0.000924 |
| GRXCR2 | 0.000923 |
| HADH | 0.000921 |
| TMEM143 | 0.000920 |
| OR5B12 | 0.000920 |
| MAGEB4 | 0.000919 |
| NSUN4 | 0.000919 |
| CST9L | 0.000919 |
| NDUFA12 | 0.000918 |
| OR5M1 | 0.000918 |
| SCAF1 | 0.000918 |
| CCDC126 | 0.000918 |
| SPDYE16 | 0.000917 |
| PSMD5 | 0.000917 |
| KRT10 | 0.000917 |
| ITGB8 | 0.000916 |
| METTL25 | 0.000915 |
| DYNLL1 | 0.000915 |
| PRAMEF10 | 0.000915 |
| PRAMEF17 | 0.000914 |
| CYP11B2 | 0.000914 |
| FIBP | 0.000913 |
| OR6C2 | 0.000912 |
| FBXW2 | 0.000912 |
| ELP5 | 0.000912 |
| LOR | 0.000912 |
| BTRC | 0.000912 |
| DLAT | 0.000911 |
| A2ML1 | 0.000911 |
| EDN2 | 0.000911 |
| OR6B3 | 0.000911 |
| OR5F1 | 0.000910 |
| DET1 | 0.000910 |
| CABP2 | 0.000909 |
| OR2T10 | 0.000909 |
| LRRC28 | 0.000909 |
| XPA | 0.000908 |
| PRSS38 | 0.000908 |
| RAD51B | 0.000908 |
| SLC25A46 | 0.000907 |
| PRAMEF25 | 0.000906 |
| OR8J2 | 0.000905 |
| ZNF526 | 0.000905 |
| OR10AC1 | 0.000905 |
| OR5H1 | 0.000905 |
| GUCY2C | 0.000904 |
| TMUB2 | 0.000904 |
| SFMBT2 | 0.000903 |
| C14orf177 | 0.000903 |
| NDUFS3 | 0.000902 |
| OR5D13 | 0.000901 |
| TMEM254 | 0.000900 |
| RAPGEF6 | 0.000900 |
| DHRSX | 0.000900 |
| OR8G5 | 0.000900 |
| COPB2 | 0.000900 |
| PTDSS2 | 0.000899 |
| ATXN3L | 0.000899 |
| SNX4 | 0.000899 |
| GFPT1 | 0.000899 |
| ADO | 0.000899 |
| MED13L | 0.000899 |
| FOXP4 | 0.000898 |
| SRRD | 0.000898 |
| DDX1 | 0.000897 |
| SENP8 | 0.000897 |
| KIR3DL3 | 0.000896 |
| KRTAP9-6 | 0.000895 |
| C9orf92 | 0.000895 |
| IPMK | 0.000895 |
| ZPBP2 | 0.000894 |
| ZNF668 | 0.000892 |
| IFNA2 | 0.000891 |
| KIFAP3 | 0.000889 |
| AGBL2 | 0.000889 |
| ZNF213 | 0.000887 |
| STRAP | 0.000886 |
| LTV1 | 0.000886 |
| NAGK | 0.000886 |
| NUBPL | 0.000886 |
| NXPE4 | 0.000886 |
| NFX1 | 0.000885 |
| METTL15 | 0.000885 |
| FAM103A1 | 0.000885 |
| WARS2 | 0.000884 |
| ASB17 | 0.000884 |
| PLXNA1 | 0.000884 |
| MRPS35 | 0.000883 |
| OR8J1 | 0.000883 |
| NOA1 | 0.000882 |
| HECTD1 | 0.000882 |
| CBFA2T3 | 0.000882 |
| USE1 | 0.000881 |
| COTL1 | 0.000881 |
| SETSIP | 0.000881 |
| SLC51A | 0.000880 |
| BLID | 0.000880 |
| QRFPR | 0.000880 |
| OR8H3 | 0.000880 |
| ACCSL | 0.000879 |
| PRM2 | 0.000879 |
| KRTAP19-7 | 0.000877 |
| SLC35E4 | 0.000877 |
| PPCDC | 0.000877 |
| MMP20 | 0.000876 |
| DERL2 | 0.000876 |
| TMCC1 | 0.000875 |
| LRRC30 | 0.000875 |
| ZCCHC10 | 0.000875 |
| GEMIN8 | 0.000874 |
| ZP4 | 0.000873 |
| UQCRQ | 0.000873 |
| ALDOC | 0.000872 |
| AK6 | 0.000872 |
| MAP4K2 | 0.000872 |
| DKK4 | 0.000872 |
| EPPK1 | 0.000871 |
| OR4A47 | 0.000870 |
| TMEM56 | 0.000870 |
| SERPINI2 | 0.000870 |
| IFNA6 | 0.000869 |
| HRNR | 0.000869 |
| C20orf24 | 0.000869 |
| AIMP2 | 0.000869 |
| FGF3 | 0.000868 |
| CDK17 | 0.000868 |
| LAMA1 | 0.000868 |
| KRTAP19-5 | 0.000867 |
| KRTAP6-1 | 0.000863 |
| TAAR9 | 0.000863 |
| ZCCHC9 | 0.000863 |
| ARHGEF40 | 0.000862 |
| IFNA14 | 0.000862 |
| OBP2A | 0.000860 |
| BTNL2 | 0.000859 |
| CXCR4 | 0.000858 |
| UHMK1 | 0.000858 |
| CWC22 | 0.000857 |
| MOCS3 | 0.000856 |
| OR51B5 | 0.000856 |
| OGFOD2 | 0.000856 |
| DNAJC21 | 0.000855 |
| BCAS2 | 0.000855 |
| HSPBP1 | 0.000854 |
| GNA11 | 0.000851 |
| TCTN2 | 0.000850 |
| ZNF581 | 0.000850 |
| P3H4 | 0.000850 |
| KCNK3 | 0.000849 |
| PGAP2 | 0.000849 |
| USP10 | 0.000849 |
| FAM204A | 0.000849 |
| KRTAP6-3 | 0.000849 |
| ZBTB45 | 0.000848 |
| TMEM230 | 0.000848 |
| CDR1 | 0.000848 |
| KRTAP12-4 | 0.000848 |
| DYNC2LI1 | 0.000847 |
| GTF2IRD2B | 0.000847 |
| OR4C3 | 0.000847 |
| MAGED1 | 0.000846 |
| TMEM126B | 0.000845 |
| KRTAP4-8 | 0.000844 |
| ZNF609 | 0.000844 |
| ICMT | 0.000843 |
| MPV17L2 | 0.000843 |
| MAPK7 | 0.000843 |
| LARS2 | 0.000843 |
| ERCC4 | 0.000842 |
| KRTAP4-3 | 0.000842 |
| NCL | 0.000842 |
| MAP3K2 | 0.000842 |
| KPNA4 | 0.000841 |
| AQR | 0.000841 |
| CRKL | 0.000841 |
| ACSM4 | 0.000841 |
| UCMA | 0.000841 |
| HABP4 | 0.000840 |
| FST | 0.000839 |
| CRNN | 0.000839 |
| LSM1 | 0.000839 |
| OR6C70 | 0.000838 |
| PSMA7 | 0.000838 |
| COX8A | 0.000838 |
| PRAMEF20 | 0.000837 |
| PRAMEF11 | 0.000836 |
| FTMT | 0.000836 |
| EXOC1 | 0.000834 |
| HNRNPCL3 | 0.000834 |
| CLDN24 | 0.000834 |
| MTRNR2L10 | 0.000834 |
| OR10G8 | 0.000833 |
| SSX7 | 0.000833 |
| DDA1 | 0.000833 |
| ATP5D | 0.000833 |
| HARS | 0.000833 |
| OR4C46 | 0.000832 |
| RABGAP1 | 0.000832 |
| CWF19L1 | 0.000832 |
| SRGAP2C | 0.000832 |
| THADA | 0.000831 |
| SLC35G4 | 0.000831 |
| SEMG2 | 0.000831 |
| OR2Z1 | 0.000830 |
| IQCG | 0.000830 |
| TNP03 | 0.000830 |
| SNF8 | 0.000829 |
| ETV3L | 0.000829 |
| OR5AC2 | 0.000828 |
| KLRC2 | 0.000828 |
| APC | 0.000828 |
| ZCCHC17 | 0.000828 |
| SERPINA4 | 0.000828 |
| OR5L1 | 0.000828 |
| OR8H2 | 0.000827 |
| SLC15A4 | 0.000827 |
| IER3IP1 | 0.000826 |
| PGK1 | 0.000826 |
| PSMA4 | 0.000826 |
| IFNA16 | 0.000824 |
| STK32B | 0.000824 |
| RASA4 | 0.000823 |
| CWC25 | 0.000823 |
| PDSS2 | 0.000823 |
| UBALD1 | 0.000822 |
| IFT43 | 0.000822 |
| SLC17A3 | 0.000822 |
| OR51I1 | 0.000821 |
| COX16 | 0.000820 |
| OR4E1 | 0.000820 |
| CDC42BPA | 0.000820 |
| TEX13A | 0.000816 |
| ST3GAL3 | 0.000815 |
| DPPA5 | 0.000815 |
| SAGE1 | 0.000814 |
| NKAP | 0.000814 |
| HOOK3 | 0.000813 |
| CT62 | 0.000812 |
| UBIAD1 | 0.000811 |
| DHODH | 0.000811 |
| YTHDF3 | 0.000811 |
| DNAJB6 | 0.000811 |
| TSPAN5 | 0.000809 |
| SPACA5 | 0.000809 |
| OR5AL1 | 0.000809 |
| ACTRT2 | 0.000809 |
| NIPA2 | 0.000809 |
| PABPC4 | 0.000809 |
| SPATA45 | 0.000808 |
| OR11G2 | 0.000808 |
| OR5T1 | 0.000808 |
| TRDMT1 | 0.000807 |
| NDUFS8 | 0.000807 |
| AMTN | 0.000807 |
| SPCS2 | 0.000807 |
| PRELID2 | 0.000806 |
| CARNS1 | 0.000806 |
| OR2Y1 | 0.000806 |
| USP3 | 0.000805 |
| DCAF8 | 0.000805 |
| ZNF391 | 0.000805 |
| UBR2 | 0.000805 |
| GPR119 | 0.000804 |
| N4BP2L1 | 0.000804 |
| PARL | 0.000804 |
| OR51V1 | 0.000804 |
| POFUT1 | 0.000802 |
| GJA8 | 0.000802 |
| CD200R1L | 0.000801 |
| FCRL4 | 0.000800 |
| UBQLN4 | 0.000800 |
| MDH1 | 0.000800 |
| CLCN3 | 0.000799 |
| ZBTB43 | 0.000799 |
| ROMO1 | 0.000798 |
| SQLE | 0.000797 |
| PITRM1 | 0.000797 |
| ASB7 | 0.000795 |
| ALPI | 0.000795 |
| TTC39B | 0.000794 |
| TAS2R60 | 0.000794 |
| OR2T11 | 0.000793 |
| RBM23 | 0.000792 |
| SLC27A4 | 0.000792 |
| TUBAL3 | 0.000792 |
| C16orf91 | 0.000791 |
| OR13C9 | 0.000791 |
| WDR55 | 0.000790 |
| EFNA4 | 0.000789 |
| OR10Q1 | 0.000789 |
| BFSP2 | 0.000788 |
| OR4C13 | 0.000788 |
| PRR23A | 0.000786 |
| PRKACG | 0.000786 |
| NSMAF | 0.000785 |
| ZNF25 | 0.000784 |
| OR1E1 | 0.000784 |
| RAD23A | 0.000783 |
| MRPL51 | 0.000783 |
| LRCH4 | 0.000783 |
| SUMO1 | 0.000783 |
| OR8I2 | 0.000782 |
| RIC1 | 0.000780 |
| MRLN | 0.000780 |
| ASAP1 | 0.000778 |
| OR5H14 | 0.000778 |
| PBLD | 0.000777 |
| METTL9 | 0.000777 |
| OR5R1 | 0.000774 |
| POF1B | 0.000774 |
| JRKL | 0.000774 |
| ATG13 | 0.000773 |
| HSF1 | 0.000773 |
| CHCHD1 | 0.000773 |
| VCAN | 0.000773 |
| HTR3D | 0.000773 |
| ATG16L1 | 0.000772 |
| DCP1B | 0.000772 |
| KLC4 | 0.000772 |
| DLST | 0.000772 |
| NUP214 | 0.000771 |
| OR1S1 | 0.000770 |
| ETFDH | 0.000769 |
| BTN2A1 | 0.000769 |
| KIAA0922 | 0.000768 |
| PRAMEF4 | 0.000766 |
| OR8U1 | 0.000766 |
| TMEM218 | 0.000766 |
| TRAF6 | 0.000766 |
| GPR143 | 0.000765 |
| KRT3 | 0.000765 |
| YARS | 0.000765 |
| AXDND1 | 0.000764 |
| NDUFC2 | 0.000764 |
| DAOA | 0.000763 |
| STXBP3 | 0.000763 |
| C18orf8 | 0.000763 |
| OR5T3 | 0.000762 |
| KHNYN | 0.000762 |
| QPCTL | 0.000761 |
| SUN3 | 0.000761 |
| OSBP | 0.000760 |
| KRTAP8-1 | 0.000760 |
| RASSF10 | 0.000759 |
| ZNF408 | 0.000759 |
| PEMT | 0.000758 |
| CT45A2 | 0.000758 |
| SMO | 0.000758 |
| RNF40 | 0.000758 |
| DCAF4 | 0.000758 |
| SLC7A6OS | 0.000757 |
| HDAC5 | 0.000756 |
| MBD3L4 | 0.000756 |
| PRKACA | 0.000756 |
| TMEM39A | 0.000756 |
| CCNH | 0.000755 |
| RNF20 | 0.000755 |
| PNRC2 | 0.000755 |
| MRPL55 | 0.000754 |
| C7orf26 | 0.000754 |
| PDE9A | 0.000754 |
| KRTAP4-6 | 0.000754 |
| MT4 | 0.000754 |
| DGAT1 | 0.000753 |
| RBMY1E | 0.000753 |
| KDM3B | 0.000750 |
| PRAF2 | 0.000750 |
| EDARADD | 0.000750 |
| SPANXD | 0.000749 |
| CNPY2 | 0.000748 |
| AIFM3 | 0.000747 |
| CUL1 | 0.000746 |
| VEZT | 0.000746 |
| ACTR1B | 0.000745 |
| TYW3 | 0.000745 |
| F5 | 0.000745 |
| ATG3 | 0.000745 |
| COQ9 | 0.000745 |
| IFT46 | 0.000745 |
| RAD50 | 0.000744 |
| KRTAP15-1 | 0.000744 |
| CYP27C1 | 0.000744 |
| PIGO | 0.000744 |
| HMCES | 0.000744 |
| PINLYP | 0.000743 |
| SH2B2 | 0.000742 |
| SEMG1 | 0.000741 |
| IGFL3 | 0.000740 |
| BROX | 0.000740 |
| SLC2A8 | 0.000740 |
| ACTL9 | 0.000740 |
| SKIV2L2 | 0.000740 |
| SLC22A5 | 0.000739 |
| C14orf28 | 0.000738 |
| ABRACL | 0.000737 |
| PUF60 | 0.000737 |
| OR51B4 | 0.000737 |
| DEDD2 | 0.000737 |
| ADAM18 | 0.000736 |
| OR7A10 | 0.000736 |
| C9orf78 | 0.000736 |
| RALBP1 | 0.000735 |
| OR7G1 | 0.000735 |
| MT-ND4L | 0.000733 |
| HTR1F | 0.000731 |
| TEX14 | 0.000729 |
| SLC30A9 | 0.000728 |
| SLC7A6 | 0.000728 |
| LRRC17 | 0.000728 |
| BANF2 | 0.000727 |
| DBT | 0.000726 |
| OR1D4 | 0.000726 |
| PRDM2 | 0.000725 |
| WDR92 | 0.000722 |
| ZNF658 | 0.000721 |
| KRTAP4-4 | 0.000721 |
| AURKAIP1 | 0.000721 |
| BANF1 | 0.000721 |
| OR10G7 | 0.000720 |
| PIH1D2 | 0.000720 |
| PNP | 0.000720 |
| TTLL9 | 0.000720 |
| LRRC1 | 0.000719 |
| RIBC1 | 0.000718 |
| B4GALT5 | 0.000717 |
| ELOVL5 | 0.000717 |
| METTL14 | 0.000717 |
| ZDHHC20 | 0.000716 |
| AMOT | 0.000716 |
| MED29 | 0.000715 |
| SLC38A9 | 0.000715 |
| C3orf58 | 0.000714 |
| EFCAB3 | 0.000714 |
| SLC24A5 | 0.000714 |
| ZCCHC2 | 0.000714 |
| NR2F6 | 0.000712 |
| OSBPL8 | 0.000710 |
| OR1A1 | 0.000710 |
| ADAM19 | 0.000710 |
| AGAP3 | 0.000709 |
| PPP3CA | 0.000709 |
| FBXW10 | 0.000709 |
| DEFB108B | 0.000707 |
| CLDN17 | 0.000705 |
| DENND6A | 0.000705 |
| ACVR1B | 0.000705 |
| KRTAP9-1 | 0.000704 |
| RNF148 | 0.000703 |
| PITX3 | 0.000703 |
| OR10G3 | 0.000701 |
| OR12D2 | 0.000701 |
| ACOT6 | 0.000700 |
| PRAMEF6 | 0.000700 |
| OR4A16 | 0.000699 |
| TRIQK | 0.000698 |
| COG6 | 0.000697 |
| OR2V2 | 0.000697 |
| ZNF362 | 0.000697 |
| BHLHE22 | 0.000697 |
| RPP25 | 0.000697 |
| MT-ATP6 | 0.000696 |
| OXSR1 | 0.000696 |
| RNF103 | 0.000695 |
| PPP1R21 | 0.000694 |
| OR5B17 | 0.000693 |
| APEX2 | 0.000692 |
| UBL5 | 0.000692 |
| ABHD16A | 0.000692 |
| OR2AP1 | 0.000692 |
| SPINK9 | 0.000691 |
| HIGD1C | 0.000689 |
| AADACL2 | 0.000688 |
| TUBB4B | 0.000688 |
| BNIP1 | 0.000688 |
| FTHL17 | 0.000688 |
| PRORY | 0.000687 |
| RPL35A | 0.000687 |
| PRSS58 | 0.000687 |
| WIPE | 0.000686 |
| SLC35C2 | 0.000686 |
| RNF25 | 0.000685 |
| IPO7 | 0.000685 |
| IL9 | 0.000685 |
| SPATA6 | 0.000685 |
| MRPL17 | 0.000685 |
| TMEM244 | 0.000684 |
| OR7E24 | 0.000683 |
| METTL16 | 0.000682 |
| MRPS15 | 0.000682 |
| CDRT15L2 | 0.000681 |
| TNKS2 | 0.000680 |
| EXOC2 | 0.000680 |
| MGAT5 | 0.000679 |
| PQLC2 | 0.000678 |
| OR5AP2 | 0.000677 |
| TSNAX | 0.000677 |
| UBOX5 | 0.000677 |
| BMP2K | 0.000676 |
| IGSF22 | 0.000675 |
| PRKCI | 0.000675 |
| KCNE4 | 0.000674 |
| GPR137 | 0.000673 |
| KIAA0430 | 0.000672 |
| TEX13B | 0.000672 |
| MXD1 | 0.000671 |
| SERHL2 | 0.000671 |
| CACUL1 | 0.000671 |
| DYNC1LI2 | 0.000669 |
| KRT28 | 0.000669 |
| OR2M7 | 0.000668 |
| ACTR10 | 0.000668 |
| CELA3B | 0.000667 |
| SEC63 | 0.000667 |
| CRYGD | 0.000666 |
| SLC44A1 | 0.000666 |
| C1orf64 | 0.000666 |
| OR4S2 | 0.000666 |
| BRPF1 | 0.000666 |
| ARIH1 | 0.000665 |
| RINL | 0.000664 |
| RAB22A | 0.000664 |
| GAGE12B | 0.000663 |
| IFT140 | 0.000663 |
| SLC35B1 | 0.000663 |
| C5orf24 | 0.000662 |
| C12orf66 | 0.000662 |
| RPL17 | 0.000661 |
| C1QBP | 0.000661 |
| CTBP2 | 0.000661 |
| FOXK1 | 0.000660 |
| MFSD11 | 0.000660 |
| RNASE11 | 0.000658 |
| NDUFA8 | 0.000658 |
| ADGRF4 | 0.000658 |
| PDPK1 | 0.000658 |
| XDH | 0.000657 |
| DHX8 | 0.000657 |
| C12orf57 | 0.000657 |
| TM2D3 | 0.000657 |
| VCY | 0.000656 |
| CEPT1 | 0.000655 |
| MOS | 0.000655 |
| GLT6D1 | 0.000655 |
| DENND4A | 0.000654 |
| C1GALT1 | 0.000653 |
| ATP13A2 | 0.000653 |
| BAG6 | 0.000653 |
| NCALD | 0.000653 |
| CCDC159 | 0.000652 |
| SPRY3 | 0.000652 |
| OR7G3 | 0.000652 |
| MOSPD1 | 0.000651 |
| HSPA8 | 0.000651 |
| LIPC | 0.000650 |
| MTSS1L | 0.000647 |
| SLC22A9 | 0.000647 |
| TMEM55A | 0.000646 |
| LUZP4 | 0.000645 |
| 43715 | 0.000645 |
| STRA8 | 0.000645 |
| GGPS1 | 0.000644 |
| IFNA8 | 0.000644 |
| VCY1B | 0.000644 |
| SLC35A5 | 0.000644 |
| PPIAL4E | 0.000643 |
| RNF114 | 0.000643 |
| VPS4B | 0.000642 |
| TBK1 | 0.000641 |
| CREBBP | 0.000640 |
| OR8B4 | 0.000640 |
| SRP19 | 0.000640 |
| GEMIN4 | 0.000640 |
| RABGAP1L | 0.000639 |
| SPACA7 | 0.000639 |
| FAM76A | 0.000639 |
| MINOS1 | 0.000639 |
| RRP8 | 0.000639 |
| POLL | 0.000638 |
| COPRS | 0.000638 |
| ZNRD1 | 0.000638 |
| CELA2A | 0.000637 |
| SNAPCI | 0.000637 |
| OR6C68 | 0.000637 |
| TBC1D20 | 0.000636 |
| NOX3 | 0.000636 |
| SPACA1 | 0.000636 |
| OGFOD3 | 0.000636 |
| JPH1 | 0.000636 |
| C1orf228 | 0.000635 |
| OR5H15 | 0.000635 |
| COASY | 0.000634 |
| APH1B | 0.000633 |
| OR10P1 | 0.000633 |
| GAGE12C | 0.000633 |
| GAGE12D | 0.000633 |
| MPI | 0.000632 |
| KRTAP27-1 | 0.000632 |
| MGAT4B | 0.000632 |
| PDPR | 0.000632 |
| ERICH3 | 0.000631 |
| AKT1S1 | 0.000631 |
| NLRC5 | 0.000630 |
| MTDH | 0.000629 |
| CHST7 | 0.000629 |
| ARHGEF10L | 0.000629 |
| URAD | 0.000629 |
| IL1F10 | 0.000628 |
| LRRC57 | 0.000628 |
| CLUAP1 | 0.000628 |
| WDR37 | 0.000628 |
| TP53INP1 | 0.000628 |
| KDM2A | 0.000627 |
| PAF1 | 0.000627 |
| OR4C11 | 0.000626 |
| GAST | 0.000626 |
| FOPNL | 0.000623 |
| MTX1 | 0.000623 |
| AGPAT1 | 0.000623 |
| SPANXN5 | 0.000623 |
| PIGP | 0.000622 |
| MRPS34 | 0.000622 |
| NFIB | 0.000622 |
| NBPF15 | 0.000622 |
| ASNSD1 | 0.000622 |
| INS | 0.000622 |
| OR2T27 | 0.000621 |
| CDYL2 | 0.000621 |
| CFAP20 | 0.000620 |
| RABL2B | 0.000619 |
| TMEM60 | 0.000619 |
| GANAB | 0.000619 |
| ARF6 | 0.000618 |
| IBA57 | 0.000618 |
| GJA3 | 0.000617 |
| USP17L17 | 0.000617 |
| ACCS | 0.000617 |
| OR5H6 | 0.000617 |
| CMTR2 | 0.000616 |
| CLLU1OS | 0.000616 |
| MIER1 | 0.000615 |
| LYPD3 | 0.000615 |
| SERPINB4 | 0.000614 |
| FBXL13 | 0.000613 |
| C7orf72 | 0.000612 |
| SLC7A10 | 0.000612 |
| COMMD10 | 0.000612 |
| RPS19 | 0.000611 |
| STARD3 | 0.000611 |
| PTK7 | 0.000611 |
| AK3 | 0.000610 |
| PRMT9 | 0.000610 |
| ABT1 | 0.000609 |
| UBE2A | 0.000608 |
| TMEM207 | 0.000608 |
| PHYHIP | 0.000608 |
| STK3 | 0.000607 |
| HOXA2 | 0.000607 |
| CYP4F3 | 0.000607 |
| FPGT | 0.000607 |
| ZNF582 | 0.000606 |
| VPS41 | 0.000606 |
| KLKB1 | 0.000606 |
| FBLIM1 | 0.000606 |
| RXFP4 | 0.000605 |
| MAP2K2 | 0.000605 |
| IFNA7 | 0.000604 |
| UBL4A | 0.000603 |
| DNAJA1 | 0.000603 |
| NDUFS2 | 0.000603 |
| AADACL4 | 0.000602 |
| OR9G4 | 0.000601 |
| SOX15 | 0.000600 |
| IGDCC4 | 0.000599 |
| CPD | 0.000598 |
| FAT1 | 0.000598 |
| OXSM | 0.000594 |
| LY6G6D | 0.000594 |
| RDH14 | 0.000593 |
| AKAP10 | 0.000592 |
| RPS4Y2 | 0.000591 |
| MTMR3 | 0.000591 |
| HECA | 0.000591 |
| OR1M1 | 0.000591 |
| TFG | 0.000590 |
| OTUD4 | 0.000590 |
| NPHS2 | 0.000590 |
| NUTM2A | 0.000589 |
| EXOC4 | 0.000589 |
| MTFP1 | 0.000589 |
| THAP7 | 0.000588 |
| POTEB | 0.000588 |
| ING2 | 0.000586 |
| KRTAP29-1 | 0.000586 |
| OR4P4 | 0.000585 |
| OR5J2 | 0.000585 |
| BEND7 | 0.000585 |
| KRTAP9-2 | 0.000584 |
| KRTAP1-4 | 0.000584 |
| SMU1 | 0.000584 |
| LMO4 | 0.000584 |
| BICD2 | 0.000583 |
| ANKRD50 | 0.000583 |
| PHYKPL | 0.000583 |
| FOXI2 | 0.000582 |
| MRPL27 | 0.000581 |
| ATMIN | 0.000581 |
| RBM15B | 0.000581 |
| HIST1H2BA | 0.000580 |
| SPO11 | 0.000580 |
| KLHL26 | 0.000580 |
| ARPC5L | 0.000579 |
| MLN | 0.000579 |
| OR5D16 | 0.000577 |
| PIGC | 0.000576 |
| MRPS28 | 0.000576 |
| TMEM168 | 0.000576 |
| OCM2 | 0.000575 |
| ARMS2 | 0.000575 |
| SLC18B1 | 0.000575 |
| FAM209B | 0.000575 |
| DIS3 | 0.000574 |
| CDC123 | 0.000573 |
| OSBPL6 | 0.000573 |
| ZNF800 | 0.000572 |
| OR10Z1 | 0.000571 |
| GPHN | 0.000571 |
| IL3 | 0.000571 |
| MRFAP1L1 | 0.000570 |
| MKRN2 | 0.000570 |
| RORA | 0.000569 |
| ARMC10 | 0.000569 |
| RABL3 | 0.000568 |
| LMF2 | 0.000568 |
| OR6Q1 | 0.000568 |
| NAAA | 0.000567 |
| TMEM202 | 0.000567 |
| C6orf120 | 0.000566 |
| LCE1C | 0.000566 |
| RITA1 | 0.000566 |
| NDUFV2 | 0.000566 |
| SUPT3H | 0.000566 |
| MARK2 | 0.000566 |
| ELSPBP1 | 0.000565 |
| PSME2 | 0.000565 |
| GLT8D1 | 0.000565 |
| SNX19 | 0.000564 |
| AIFM1 | 0.000564 |
| SH3BGR | 0.000563 |
| SLC25A16 | 0.000563 |
| KRT6C | 0.000563 |
| FLYWCH1 | 0.000562 |
| LSM3 | 0.000562 |
| HNRNPCL4 | 0.000562 |
| SNAPIN | 0.000562 |
| JAG1 | 0.000561 |
| KRTAP5-6 | 0.000561 |
| FAEM2 | 0.000559 |
| FAM160B2 | 0.000559 |
| OR10H1 | 0.000558 |
| SLC4A1AP | 0.000557 |
| IFNE | 0.000556 |
| DENND2A | 0.000556 |
| LRRC27 | 0.000556 |
| GPX6 | 0.000556 |
| ITPR3 | 0.000556 |
| SMIM9 | 0.000554 |
| ZNF641 | 0.000554 |
| AUP1 | 0.000554 |
| AFTPH | 0.000553 |
| OR10AD1 | 0.000553 |
| KIAA1551 | 0.000553 |
| HEATR3 | 0.000552 |
| MRPL44 | 0.000552 |
| ATP5L | 0.000552 |
| AKT1 | 0.000551 |
| HR | 0.000551 |
| DDB1 | 0.000551 |
| TNR | 0.000548 |
| RGR | 0.000547 |
| FAM184A | 0.000547 |
| OR5AS1 | 0.000547 |
| NEK8 | 0.000546 |
| NADK | 0.000546 |
| OR2T35 | 0.000546 |
| GTPBP6 | 0.000545 |
| DNAJB5 | 0.000545 |
| VPS25 | 0.000545 |
| DEFB107A | 0.000541 |
| DEFB107B | 0.000541 |
| CCDC124 | 0.000540 |
| BIRC2 | 0.000540 |
| SMR3A | 0.000539 |
| GRXCR1 | 0.000538 |
| XIAP | 0.000538 |
| GMNC | 0.000538 |
| NAV2 | 0.000537 |
| TGM5 | 0.000537 |
| RPL26L1 | 0.000537 |
| MEIG1 | 0.000536 |
| PDGFD | 0.000536 |
| LYST | 0.000535 |
| IQCF6 | 0.000535 |
| PDILT | 0.000535 |
| C16orf90 | 0.000535 |
| FGF6 | 0.000534 |
| TMTC1 | 0.000534 |
| C7orf49 | 0.000534 |
| DEF8 | 0.000533 |
| ARPP19 | 0.000533 |
| MIEF1 | 0.000533 |
| INAFM1 | 0.000532 |
| MORN2 | 0.000532 |
| LBP | 0.000532 |
| ZC3H12B | 0.000532 |
| MYBBP1A | 0.000531 |
| RFPL4A | 0.000531 |
| SLC52A2 | 0.000531 |
| KRT25 | 0.000531 |
| KRT33B | 0.000530 |
| CABIN1 | 0.000530 |
| FBXL14 | 0.000529 |
| CGRRF1 | 0.000527 |
| BBS9 | 0.000527 |
| SLC43A1 | 0.000526 |
| IMPAD1 | 0.000526 |
| RFWD2 | 0.000526 |
| TMEM161A | 0.000525 |
| POTEB2 | 0.000525 |
| DUSP19 | 0.000524 |
| TMEM126A | 0.000523 |
| TBC1D3H | 0.000523 |
| SLC22A2 | 0.000523 |
| NUDT5 | 0.000522 |
| SPATA31A5 | 0.000522 |
| NDUFA6 | 0.000522 |
| HINT1 | 0.000521 |
| COPS4 | 0.000521 |
| BZW2 | 0.000520 |
| ATG4C | 0.000519 |
| PSKH2 | 0.000518 |
| RPH3A | 0.000518 |
| PHPT1 | 0.000518 |
| TRAPPC3 | 0.000516 |
| RAB40C | 0.000515 |
| CLPP | 0.000514 |
| CCM2 | 0.000514 |
| GOLGA2 | 0.000513 |
| GRIFIN | 0.000513 |
| KRTAP19-6 | 0.000513 |
| EIF3M | 0.000512 |
| PAGE3 | 0.000512 |
| ZNF341 | 0.000512 |
| YTHDF2 | 0.000512 |
| AMOTL1 | 0.000511 |
| SNN | 0.000511 |
| BFSP1 | 0.000510 |
| NOP14 | 0.000509 |
| TRPV1 | 0.000507 |
| STAT5B | 0.000506 |
| TMEM135 | 0.000506 |
| KRT37 | 0.000505 |
| PTPN20 | 0.000505 |
| LRIT1 | 0.000505 |
| PITPNB | 0.000503 |
| BTBD6 | 0.000503 |
| DRG1 | 0.000503 |
| REG3A | 0.000502 |
| OR10G9 | 0.000502 |
| CCT7 | 0.000502 |
| RIOK3 | 0.000502 |
| FAF2 | 0.000501 |
| SLC22A6 | 0.000501 |
| TMEM258 | 0.000501 |
| OR4C16 | 0.000501 |
| OR4D5 | 0.000500 |
| AMBRA1 | 0.000500 |
| DEFBI19 | 0.000500 |
| ZC3H3 | 0.000500 |
| HMGN4 | 0.000500 |
| CRYGB | 0.000499 |
| OR10X1 | 0.000498 |
| RARS | 0.000497 |
| TOMM34 | 0.000497 |
| MARVELD3 | 0.000496 |
| TBC1D14 | 0.000496 |
| POLR2F | 0.000496 |
| TBC1D1 | 0.000495 |
| LARP7 | 0.000494 |
| C2CD2L | 0.000494 |
| BBS1 | 0.000494 |
| PCGF1 | 0.000493 |
| PRMT8 | 0.000493 |
| SPATA3 | 0.000492 |
| C1orf123 | 0.000491 |
| OR51B2 | 0.000489 |
| FASTKD5 | 0.000489 |
| SMARCD2 | 0.000489 |
| RBMY1J | 0.000489 |
| DAZL | 0.000489 |
| KRTAP4-9 | 0.000488 |
| C20orf196 | 0.000488 |
| RAF1 | 0.000488 |
| PSMB4 | 0.000487 |
| ZNF747 | 0.000486 |
| APOBEC1 | 0.000486 |
| SMARCAL1 | 0.000486 |
| PEX10 | 0.000486 |
| RNF4 | 0.000485 |
| PLA2G2E | 0.000485 |
| SEMA4F | 0.000484 |
| LCE2C | 0.000483 |
| MOB4 | 0.000481 |
| ATF4 | 0.000481 |
| DEFB121 | 0.000481 |
| FBX033 | 0.000481 |
| HIF1A | 0.000481 |
| C2orf76 | 0.000480 |
| ZNF777 | 0.000480 |
| MBD3L1 | 0.000480 |
| FBXO28 | 0.000479 |
| PLEKHO1 | 0.000478 |
| ITPA | 0.000478 |
| SUPT20HL1 | 0.000477 |
| ZNF114 | 0.000476 |
| RNF111 | 0.000476 |
| OR4D9 | 0.000475 |
| CENPB | 0.000474 |
| GTF2F1 | 0.000474 |
| ERCC1 | 0.000473 |
| RGMB | 0.000472 |
| RPS15 | 0.000472 |
| RASA1 | 0.000470 |
| EEFSEC | 0.000470 |
| HNF1A | 0.000470 |
| GPR155 | 0.000469 |
| CNTN5 | 0.000468 |
| SPRR2E | 0.000468 |
| H2AFB1 | 0.000468 |
| PSMD6 | 0.000468 |
| SRFBP1 | 0.000468 |
| AIDA | 0.000467 |
| SYCE1 | 0.000467 |
| DENND4C | 0.000467 |
| OR5T2 | 0.000466 |
| FMNL2 | 0.000466 |
| CT45A7 | 0.000466 |
| KRTAP4-2 | 0.000464 |
| ZNF816 | 0.000463 |
| CRABP2 | 0.000459 |
| SLC38A6 | 0.000459 |
| CTAGE9 | 0.000459 |
| UGGT1 | 0.000458 |
| PHF5A | 0.000458 |
| OR10V1 | 0.000458 |
| RPS21 | 0.000458 |
| CSTF2T | 0.000458 |
| PRR21 | 0.000458 |
| TMA7 | 0.000457 |
| UCHL3 | 0.000457 |
| PDCL3 | 0.000456 |
| CDS2 | 0.000455 |
| KRTAP6-2 | 0.000454 |
| VPS37C | 0.000453 |
| PSMC3 | 0.000453 |
| POLR3C | 0.000453 |
| LCA5L | 0.000453 |
| APMAP | 0.000452 |
| FRG1 | 0.000452 |
| TREM39 | 0.000452 |
| OR4X1 | 0.000451 |
| LCE5A | 0.000451 |
| WDR25 | 0.000450 |
| SLC35B3 | 0.000449 |
| EXOSC1 | 0.000448 |
| RAB2A | 0.000447 |
| KRT71 | 0.000446 |
| AP2S1 | 0.000446 |
| SCP2D1 | 0.000446 |
| TAF1B | 0.000445 |
| SCRN2 | 0.000445 |
| EPYC | 0.000444 |
| TTYH2 | 0.000444 |
| 43709 | 0.000444 |
| MOSPD2 | 0.000444 |
| LNPEP | 0.000444 |
| PCGF5 | 0.000443 |
| RNF146 | 0.000443 |
| NUDC | 0.000442 |
| TARM1 | 0.000442 |
| CT45A8 | 0.000441 |
| LEUTX | 0.000440 |
| OR10S1 | 0.000439 |
| SLC45A3 | 0.000438 |
| CUEDC2 | 0.000438 |
| HEY1 | 0.000438 |
| SEC16A | 0.000438 |
| CNBP | 0.000438 |
| SLC22A12 | 0.000437 |
| KIAA2013 | 0.000437 |
| C2orf88 | 0.000437 |
| TCEAL8 | 0.000436 |
| PPP1R1A | 0.000435 |
| UFL1 | 0.000434 |
| EFCAB9 | 0.000434 |
| ZCCHC13 | 0.000434 |
| OR8K3 | 0.000433 |
| CENPBD1 | 0.000433 |
| USP17L3 | 0.000432 |
| RPAP1 | 0.000432 |
| FGF13 | 0.000432 |
| POGK | 0.000431 |
| PRAMEF19 | 0.000431 |
| MAGEB3 | 0.000431 |
| C12orf71 | 0.000431 |
| VCX2 | 0.000431 |
| PRKCSH | 0.000431 |
| CST11 | 0.000430 |
| TMEM221 | 0.000430 |
| MAN1B1 | 0.000429 |
| KRTAP19-8 | 0.000429 |
| SIGLECL1 | 0.000428 |
| ARHGAPI2 | 0.000428 |
| LRRC72 | 0.000428 |
| LYZL1 | 0.000427 |
| MATIA | 0.000427 |
| ATP6VID | 0.000427 |
| TPMT | 0.000425 |
| ATP5G3 | 0.000425 |
| PAPPA2 | 0.000425 |
| TOR1A | 0.000425 |
| KTN1 | 0.000424 |
| KIAA1210 | 0.000424 |
| URGCP | 0.000424 |
| OR7C2 | 0.000422 |
| ZDHHC15 | 0.000421 |
| HAGH | 0.000421 |
| FAM105A | 0.000420 |
| MBLAC2 | 0.000419 |
| ACAA2 | 0.000419 |
| TTLL6 | 0.000419 |
| IL22 | 0.000418 |
| TMEM225 | 0.000417 |
| KRTAP4-5 | 0.000417 |
| IRS2 | 0.000417 |
| GDPD3 | 0.000417 |
| ZPBP | 0.000417 |
| TRAF1 | 0.000416 |
| BBX | 0.000416 |
| TMEM216 | 0.000415 |
| FZD8 | 0.000415 |
| PRG3 | 0.000414 |
| TEX36 | 0.000414 |
| NUS1 | 0.000413 |
| TIGD6 | 0.000413 |
| G3BP2 | 0.000413 |
| ANAPC10 | 0.000412 |
| OR13C7 | 0.000410 |
| THUMPD1 | 0.000410 |
| INTS12 | 0.000410 |
| FTSJ3 | 0.000408 |
| EMD | 0.000407 |
| DYNC1I2 | 0.000406 |
| DDX18 | 0.000406 |
| CD2AP | 0.000406 |
| DSCR3 | 0.000406 |
| TGIF1 | 0.000406 |
| TAF8 | 0.000405 |
| SDHAF2 | 0.000405 |
| FSCN1 | 0.000404 |
| SPRR2B | 0.000403 |
| CCDC106 | 0.000403 |
| TATDN2 | 0.000403 |
| ATP6V1A | 0.000402 |
| KRT33A | 0.000402 |
| RAP1GDS1 | 0.000401 |
| WDR46 | 0.000401 |
| USP12 | 0.000400 |
| CXorf49B | 0.000400 |
| SLC25A6 | 0.000399 |
| SIGIRR | 0.000398 |
| AMELY | 0.000398 |
| AIMP1 | 0.000398 |
| MROH8 | 0.000397 |
| FAM206A | 0.000396 |
| DGKD | 0.000395 |
| NEU2 | 0.000395 |
| C9orf69 | 0.000395 |
| TP53BP2 | 0.000394 |
| MALSU1 | 0.000393 |
| MRPL20 | 0.000392 |
| MOBP | 0.000392 |
| EIF3J | 0.000392 |
| SLC19A2 | 0.000392 |
| C3orf38 | 0.000392 |
| AKIRIN2 | 0.000391 |
| SMR3B | 0.000390 |
| NDUFB7 | 0.000389 |
| OR6C6 | 0.000388 |
| HIBCH | 0.000387 |
| 43722 | 0.000387 |
| BTBD1 | 0.000387 |
| JMJD4 | 0.000387 |
| TOR3A | 0.000387 |
| NDUFB5 | 0.000386 |
| IFT172 | 0.000385 |
| KRTAP19-4 | 0.000385 |
| IQCF5 | 0.000385 |
| CHD3 | 0.000384 |
| MTCH1 | 0.000384 |
| CCL1 | 0.000384 |
| JTB | 0.000383 |
| SULF2 | 0.000382 |
| ANKRD37 | 0.000382 |
| MMP24 | 0.000381 |
| ERMP1 | 0.000381 |
| SPANXN3 | 0.000380 |
| GTF2H1 | 0.000380 |
| RPL22 | 0.000378 |
| ALG2 | 0.000378 |
| TMEM8C | 0.000378 |
| WDFY1 | 0.000378 |
| MRPS2 | 0.000377 |
| QRICH1 | 0.000377 |
| PSMD4 | 0.000377 |
| TRIM64C | 0.000377 |
| ACADVL | 0.000376 |
| LGALS8 | 0.000376 |
| CAMSAP2 | 0.000375 |
| TMEM170A | 0.000375 |
| ATG10 | 0.000375 |
| ADIG | 0.000375 |
| RPS19BP1 | 0.000374 |
| PRPF6 | 0.000374 |
| DEFB128 | 0.000373 |
| UXT | 0.000373 |
| NUDT13 | 0.000373 |
| ZMIZ1 | 0.000372 |
| TIMM23 | 0.000371 |
| CHRNB1 | 0.000371 |
| ARL15 | 0.000371 |
| ATG5 | 0.000370 |
| SPACA5B | 0.000370 |
| MICU3 | 0.000370 |
| CNTNAP3B | 0.000370 |
| SIPA1L1 | 0.000369 |
| RPL6 | 0.000368 |
| COPB1 | 0.000368 |
| IFNL3 | 0.000367 |
| YWHAE | 0.000367 |
| MED8 | 0.000367 |
| CINP | 0.000366 |
| CC2D1B | 0.000364 |
| ATP5A1 | 0.000364 |
| SPDYC | 0.000364 |
| FAM25E | 0.000363 |
| SAA4 | 0.000363 |
| OR4X2 | 0.000363 |
| SYT12 | 0.000363 |
| PLRG1 | 0.000362 |
| SEC61A1 | 0.000362 |
| HNRNPCL1 | 0.000362 |
| YME1L1 | 0.000360 |
| SUCLA2 | 0.000360 |
| MED18 | 0.000360 |
| LLPH | 0.000357 |
| CIC | 0.000356 |
| LRRC4C | 0.000356 |
| DEFB129 | 0.000355 |
| RPL21 | 0.000354 |
| IDH3G | 0.000354 |
| LONP2 | 0.000354 |
| BCAS3 | 0.000353 |
| CLCN4 | 0.000352 |
| PKD2L1 | 0.000351 |
| CT47B1 | 0.000351 |
| TCEAL4 | 0.000351 |
| RABGEF1 | 0.000349 |
| OR2V1 | 0.000349 |
| FAHD1 | 0.000349 |
| SESN3 | 0.000349 |
| LCE2D | 0.000348 |
| NUDT22 | 0.000348 |
| MLF1 | 0.000346 |
| FAM43A | 0.000346 |
| DEFB112 | 0.000346 |
| CRYGA | 0.000345 |
| PNPLA3 | 0.000345 |
| ATP6V1C1 | 0.000343 |
| FAM57A | 0.000342 |
| KCTD5 | 0.000341 |
| ARMCX6 | 0.000340 |
| PSMC5 | 0.000339 |
| LCE3E | 0.000338 |
| ZDHHC8 | 0.000338 |
| FAM24A | 0.000337 |
| VRK2 | 0.000337 |
| HSPA12A | 0.000336 |
| VAMP4 | 0.000335 |
| EXOSC4 | 0.000335 |
| REXO2 | 0.000334 |
| C22orf46 | 0.000334 |
| CLPS | 0.000333 |
| ANKRD11 | 0.000333 |
| NSFL1C | 0.000333 |
| C19orf66 | 0.000333 |
| SPDYE4 | 0.000332 |
| AFAP1 | 0.000332 |
| POU2AF1 | 0.000330 |
| CD2BP2 | 0.000330 |
| OR52K2 | 0.000330 |
| DNALI1 | 0.000328 |
| ADCY6 | 0.000328 |
| CDY1B | 0.000326 |
| PMEL | 0.000325 |
| FBXL12 | 0.000325 |
| RDH13 | 0.000323 |
| MED31 | 0.000323 |
| NTPCR | 0.000322 |
| ULK4 | 0.000322 |
| GDPD5 | 0.000321 |
| MAGI2 | 0.000321 |
| SOGA3 | 0.000321 |
| B4GALT7 | 0.000321 |
| ZNF628 | 0.000320 |
| ATG4A | 0.000320 |
| OR2F1 | 0.000320 |
| SNX15 | 0.000318 |
| REG3G | 0.000317 |
| TPCN2 | 0.000317 |
| TMEM14B | 0.000316 |
| OSBPL3 | 0.000316 |
| MPC1L | 0.000316 |
| AKR1A1 | 0.000316 |
| TMEM223 | 0.000315 |
| WDR41 | 0.000314 |
| C11orf98 | 0.000314 |
| FDXR | 0.000314 |
| ERGIC2 | 0.000313 |
| RHOV | 0.000313 |
| MED4 | 0.000312 |
| IL25 | 0.000312 |
| NANOGNB | 0.000312 |
| NHLRC3 | 0.000312 |
| FAM178B | 0.000311 |
| NOM03 | 0.000311 |
| BPIFB3 | 0.000311 |
| ZFP2 | 0.000310 |
| KRTAP2-2 | 0.000310 |
| APBB1 | 0.000309 |
| PITPNM2 | 0.000309 |
| KRTAP20-3 | 0.000309 |
| CDC37 | 0.000309 |
| STX6 | 0.000309 |
| MTTP | 0.000309 |
| CCDC185 | 0.000308 |
| TBX1 | 0.000305 |
| MAGEB5 | 0.000305 |
| VPS33A | 0.000303 |
| RNASEH2B | 0.000301 |
| MRPS27 | 0.000301 |
| GML | 0.000301 |
| POU6F1 | 0.000301 |
| RCBTB2 | 0.000299 |
| GTF2E2 | 0.000299 |
| PP2D1 | 0.000298 |
| ATP5O | 0.000298 |
| SPRR4 | 0.000298 |
| NECAB3 | 0.000296 |
| NUTM2B | 0.000296 |
| PFKFB2 | 0.000295 |
| NDUFA1 | 0.000295 |
| GPKOW | 0.000294 |
| CACNB1 | 0.000293 |
| GAGE12F | 0.000293 |
| GAGE12G | 0.000293 |
| DUSP21 | 0.000293 |
| APBB2 | 0.000293 |
| KLHL2 | 0.000292 |
| TMEM241 | 0.000292 |
| NGRN | 0.000291 |
| MTF1 | 0.000290 |
| RNF113A | 0.000290 |
| ATP5C1 | 0.000288 |
| MYCBPAP | 0.000288 |
| IQCF2 | 0.000287 |
| KCMF1 | 0.000287 |
| FABP9 | 0.000286 |
| SAP30L | 0.000286 |
| TBC1D3C | 0.000285 |
| PRY | 0.000285 |
| HIST1H2AC | 0.000284 |
| HSP90AB1 | 0.000284 |
| ZFAND3 | 0.000283 |
| BARX2 | 0.000280 |
| TTC12 | 0.000280 |
| ASCL4 | 0.000279 |
| STU | 0.000279 |
| HIST1H4G | 0.000279 |
| CASR | 0.000278 |
| FGF20 | 0.000278 |
| ALG8 | 0.000278 |
| SRPRB | 0.000278 |
| ZNF18 | 0.000277 |
| SSX8 | 0.000275 |
| SNRNP35 | 0.000275 |
| NEK11 | 0.000274 |
| POLK | 0.000272 |
| FAM25G | 0.000272 |
| PWP2 | 0.000271 |
| SMIM2 | 0.000270 |
| NAF1 | 0.000270 |
| ZDHHC18 | 0.000270 |
| LCE3C | 0.000270 |
| DEFB110 | 0.000269 |
| UBE2Q1 | 0.000269 |
| GIGYF2 | 0.000268 |
| LYPD5 | 0.000268 |
| MED26 | 0.000267 |
| ADCY3 | 0.000267 |
| C19orf12 | 0.000266 |
| POC1B | 0.000266 |
| ATP5I | 0.000264 |
| TMEM229A | 0.000264 |
| FKBP11 | 0.000262 |
| MGARP | 0.000261 |
| DYNC1LI1 | 0.000260 |
| PPIAL4F | 0.000260 |
| NCKAP1 | 0.000258 |
| KBTBD4 | 0.000258 |
| KRTAP13-4 | 0.000257 |
| CALR | 0.000256 |
| ACACA | 0.000256 |
| KIAA0391 | 0.000256 |
| CLN6 | 0.000256 |
| F8A1 | 0.000255 |
| ASIC5 | 0.000254 |
| MTRNR2L1 | 0.000254 |
| FBX031 | 0.000253 |
| MTIF3 | 0.000253 |
| TERF2 | 0.000252 |
| ZNF442 | 0.000252 |
| KRTAP21-3 | 0.000252 |
| VGLL2 | 0.000252 |
| WDR61 | 0.000252 |
| TMEM184C | 0.000252 |
| SEC61B | 0.000252 |
| LACRT | 0.000252 |
| TMX2 | 0.000251 |
| TTLL11 | 0.000251 |
| NAP1L3 | 0.000250 |
| TTLL1 | 0.000247 |
| COPE | 0.000247 |
| TXNRD1 | 0.000246 |
| SSH2 | 0.000245 |
| ZNF763 | 0.000244 |
| BEAN1 | 0.000243 |
| ARL6IP4 | 0.000242 |
| TIRAP | 0.000241 |
| BRD7 | 0.000240 |
| SF3B2 | 0.000239 |
| DIMT1 | 0.000239 |
| TMEM253 | 0.000238 |
| CACFD1 | 0.000238 |
| SPAG1 | 0.000237 |
| USP17L13 | 0.000237 |
| MRPS23 | 0.000237 |
| TGM7 | 0.000235 |
| ZNF688 | 0.000235 |
| RFX1 | 0.000235 |
| DEFBI36 | 0.000234 |
| PFN3 | 0.000234 |
| KRT80 | 0.000233 |
| MGAT4D | 0.000233 |
| BPY2 | 0.000233 |
| KSR1 | 0.000232 |
| CNOT11 | 0.000231 |
| XPNPEP1 | 0.000231 |
| PRAMEF5 | 0.000231 |
| ELOF1 | 0.000230 |
| VDAC2 | 0.000230 |
| GPX5 | 0.000229 |
| DPM2 | 0.000229 |
| R3HDM4 | 0.000227 |
| FIGLA | 0.000227 |
| OLIG1 | 0.000226 |
| TECPR2 | 0.000226 |
| TRIAPI | 0.000226 |
| MRPS7 | 0.000225 |
| RGPD2 | 0.000225 |
| NAA20 | 0.000224 |
| EFTUD2 | 0.000224 |
| USP17L10 | 0.000222 |
| EDDM3A | 0.000222 |
| VPS9D1 | 0.000221 |
| DSTYK | 0.000220 |
| DCD | 0.000219 |
| RNASE7 | 0.000218 |
| STK4 | 0.000218 |
| ADAMTS10 | 0.000218 |
| MRPS9 | 0.000217 |
| DNAH10OS | 0.000217 |
| PSMD7 | 0.000214 |
| MAGED2 | 0.000212 |
| H3F3B | 0.000212 |
| PPP3CB | 0.000210 |
| EPHA3 | 0.000210 |
| ZNF689 | 0.000210 |
| MRPL57 | 0.000209 |
| HIF1AN | 0.000209 |
| PPP1R3D | 0.000207 |
| RNF14 | 0.000206 |
| ATE1 | 0.000206 |
| DEFB106A | 0.000205 |
| KRTAP4-12 | 0.000204 |
| ADAR | 0.000204 |
| MFSD6 | 0.000204 |
| BPY2B | 0.000203 |
| BPY2C | 0.000203 |
| AKAP4 | 0.000203 |
| CTR9 | 0.000203 |
| PPY | 0.000202 |
| TSTD3 | 0.000201 |
| PIAS1 | 0.000201 |
| UBE3B | 0.000201 |
| PSMB5 | 0.000200 |
| STK31 | 0.000199 |
| IBTK | 0.000199 |
| KIF25 | 0.000198 |
| PIGQ | 0.000197 |
| TMLHE | 0.000197 |
| ITGB1BP1 | 0.000197 |
| MRPL53 | 0.000197 |
| SPAG9 | 0.000196 |
| SAMD8 | 0.000195 |
| DEFBI16 | 0.000195 |
| ALDH1B1 | 0.000195 |
| RALY | 0.000195 |
| ELAC2 | 0.000195 |
| OCSTAMP | 0.000194 |
| ASB8 | 0.000194 |
| PARD3 | 0.000194 |
| TOP1MT | 0.000193 |
| OR5AK2 | 0.000192 |
| SDCCAG8 | 0.000192 |
| GID4 | 0.000192 |
| HMHB1 | 0.000192 |
| CLRN2 | 0.000191 |
| CSGALNACT2 | 0.000189 |
| COL5A1 | 0.000189 |
| IL36A | 0.000189 |
| CHST4 | 0.000188 |
| PCDH18 | 0.000188 |
| ZC3H13 | 0.000187 |
| PRM3 | 0.000187 |
| CT47A7 | 0.000187 |
| SLC9A6 | 0.000187 |
| MECR | 0.000186 |
| RPS7 | 0.000186 |
| EIF4G1 | 0.000186 |
| PLEKHG6 | 0.000186 |
| CTDP1 | 0.000186 |
| CCNI | 0.000186 |
| DEFB104A | 0.000185 |
| DEFB104B | 0.000185 |
| F8A2 | 0.000185 |
| TFCP2L1 | 0.000185 |
| RAB36 | 0.000185 |
| KRTAP22-1 | 0.000184 |
| MS4A5 | 0.000184 |
| EML4 | 0.000184 |
| FUK | 0.000183 |
| HPCAL4 | 0.000182 |
| KCTD20 | 0.000181 |
| CST8 | 0.000180 |
| PRADC1 | 0.000177 |
| RPS28 | 0.000176 |
| EIF2S2 | 0.000176 |
| FAM120AOS | 0.000175 |
| OPN1SW | 0.000174 |
| L3MBTL4 | 0.000174 |
| ATP5H | 0.000172 |
| FAM89B | 0.000172 |
| DTNBP1 | 0.000171 |
| KRTAP19-2 | 0.000171 |
| EXOC6 | 0.000170 |
| MAPK9 | 0.000170 |
| TSR2 | 0.000169 |
| RCN1 | 0.000168 |
| ANKRD60 | 0.000168 |
| TRIP12 | 0.000168 |
| CFDP1 | 0.000167 |
| LCE4A | 0.000167 |
| POSTN | 0.000165 |
| MPHOSPH8 | 0.000164 |
| STIM2 | 0.000164 |
| PYDC2 | 0.000163 |
| TRIM48 | 0.000163 |
| USF1 | 0.000163 |
| FCRL5 | 0.000162 |
| COPS2 | 0.000162 |
| RPL37 | 0.000162 |
| PPP2R5B | 0.000162 |
| AKIP1 | 0.000161 |
| ETV6 | 0.000161 |
| SPANXN4 | 0.000161 |
| FIG4 | 0.000161 |
| TBC1D15 | 0.000160 |
| TRAF3 | 0.000160 |
| PPP2CA | 0.000160 |
| PAPPA | 0.000160 |
| LYSMD2 | 0.000158 |
| MSGN1 | 0.000157 |
| SLC12A6 | 0.000157 |
| MPP1 | 0.000156 |
| ARV1 | 0.000155 |
| DHDDS | 0.000154 |
| MT1HL1 | 0.000154 |
| RLN3 | 0.000153 |
| GOT1L1 | 0.000152 |
| SETX | 0.000152 |
| SRC | 0.000152 |
| IFNA13 | 0.000151 |
| PIGK | 0.000151 |
| SPPL3 | 0.000151 |
| NMNAT1 | 0.000150 |
| CRYBG3 | 0.000148 |
| TPST1 | 0.000148 |
| OR52B4 | 0.000148 |
| DEGS2 | 0.000148 |
| MBD3L2 | 0.000145 |
| SLC23A1 | 0.000144 |
| LAPTM4B | 0.000144 |
| LCE1D | 0.000143 |
| NPRL3 | 0.000142 |
| FUNDC2 | 0.000142 |
| USP6NL | 0.000140 |
| TTN | 0.000139 |
| COMMD5 | 0.000139 |
| OR4C6 | 0.000139 |
| KBTBD3 | 0.000139 |
| FAM47C | 0.000138 |
| USP5 | 0.000137 |
| PSMD8 | 0.000137 |
| MRPS12 | 0.000137 |
| CELA3A | 0.000137 |
| GATB | 0.000137 |
| KCTD9 | 0.000137 |
| PCSK7 | 0.000136 |
| NPR2 | 0.000136 |
| KRTAP25-1 | 0.000136 |
| ALG10B | 0.000136 |
| RWDD1 | 0.000136 |
| FAM153B | 0.000135 |
| OAZ3 | 0.000134 |
| HIVEP3 | 0.000134 |
| NOX4 | 0.000134 |
| C6orf226 | 0.000133 |
| TYSND1 | 0.000133 |
| SPON2 | 0.000133 |
| GSDMA | 0.000132 |
| RYBP | 0.000132 |
| COX10 | 0.000132 |
| NUDCD3 | 0.000131 |
| RNASE12 | 0.000131 |
| FOX03 | 0.000131 |
| TVP23C | 0.000131 |
| AMMECR1L | 0.000131 |
| PSMD1 | 0.000130 |
| SLC29A1 | 0.000129 |
| INTS9 | 0.000129 |
| MESDC1 | 0.000128 |
| NXNL1 | 0.000128 |
| TMEM134 | 0.000127 |
| RAB6A | 0.000126 |
| OR4F4 | 0.000125 |
| FAM229B | 0.000124 |
| FCHO1 | 0.000123 |
| STK24 | 0.000123 |
| SBNO2 | 0.000122 |
| SLCO1C1 | 0.000122 |
| ARPC1A | 0.000122 |
| SLC46A1 | 0.000122 |
| GCAT | 0.000122 |
| GPN1 | 0.000121 |
| NOC4L | 0.000121 |
| TECR | 0.000121 |
| MBD3L5 | 0.000120 |
| UBE2G1 | 0.000120 |
| ECD | 0.000120 |
| OR5AR1 | 0.000118 |
| ECI2 | 0.000118 |
| NEURL3 | 0.000118 |
| SEC23B | 0.000118 |
| SP1 | 0.000117 |
| NDUFA13 | 0.000117 |
| UBA1 | 0.000116 |
| GPAA1 | 0.000114 |
| CCDC63 | 0.000113 |
| GTDC1 | 0.000113 |
| CRYGC | 0.000112 |
| KBTBD13 | 0.000112 |
| LAT | 0.000110 |
| CEACAM18 | 0.000109 |
| TEX28 | 0.000109 |
| TKFC | 0.000108 |
| CDRT1 | 0.000108 |
| TBC1D21 | 0.000107 |
| PRMT2 | 0.000106 |
| PRM1 | 0.000106 |
| DOK5 | 0.000106 |
| CSNK1D | 0.000106 |
| METTL1 | 0.000104 |
| MMS19 | 0.000103 |
| RAB11B | 0.000103 |
| RSL24D1 | 0.000103 |
| KLHDC1 | 0.000102 |
| CT45A9 | 0.000102 |
| SPTLC2 | 0.000101 |
| SLC22A23 | 0.000101 |
| EIF4G2 | 0.000100 |
| DNAJC17 | 0.000099 |
| FAP | 0.000098 |
| KRTAP20-1 | 0.000098 |
| HYAL3 | 0.000098 |
| C16orf96 | 0.000097 |
| TUSC5 | 0.000097 |
| SNX6 | 0.000096 |
| PELI3 | 0.000095 |
| PRY2 | 0.000094 |
| COX6B1 | 0.000094 |
| POLDEP2 | 0.000092 |
| PRLH | 0.000092 |
| SCGB1C2 | 0.000092 |
| GATAD2A | 0.000091 |
| DEFB135 | 0.000091 |
| OR10A6 | 0.000091 |
| WNT1 | 0.000090 |
| DEFBI27 | 0.000090 |
| POMZP3 | 0.000089 |
| CCDC61 | 0.000089 |
| SNX9 | 0.000089 |
| NDUFB10 | 0.000088 |
| LCE1F | 0.000088 |
| SART1 | 0.000087 |
| MT-CO1 | 0.000085 |
| USP17L18 | 0.000084 |
| ZNF626 | 0.000084 |
| EMC6 | 0.000083 |
| TMIGD1 | 0.000080 |
| TOR1B | 0.000078 |
| FXYD6 | 0.000078 |
| STAU1 | 0.000077 |
| ATXN10 | 0.000077 |
| FAM177B | 0.000076 |
| DEFBI23 | 0.000076 |
| OR5K2 | 0.000076 |
| RPS8 | 0.000075 |
| SPANXB1 | 0.000074 |
| NCDN | 0.000074 |
| MYL10 | 0.000073 |
| COPA | 0.000073 |
| LYZL6 | 0.000072 |
| SMYD5 | 0.000072 |
| CYP20A1 | 0.000072 |
| CXorf38 | 0.000072 |
| SWSAP1 | 0.000071 |
| HIVEP1 | 0.000071 |
| ALDH16A1 | 0.000071 |
| XPO4 | 0.000071 |
| BSPH1 | 0.000071 |
| KRTAP22-2 | 0.000070 |
| LSS | 0.000070 |
| DEFB124 | 0.000069 |
| TRIM64 | 0.000069 |
| FXR2 | 0.000069 |
| RNF166 | 0.000067 |
| NMS | 0.000067 |
| GRB7 | 0.000067 |
| SLC35G1 | 0.000066 |
| ANP32D | 0.000065 |
| ARL13B | 0.000063 |
| NCOA1 | 0.000062 |
| PGPEP1 | 0.000061 |
| HNRNPCL2 | 0.000061 |
| RPP38 | 0.000060 |
| TSC22D2 | 0.000059 |
| PSME3 | 0.000058 |
| NCOA3 | 0.000057 |
| C12orf43 | 0.000057 |
| DEFB114 | 0.000057 |
| EPS8L3 | 0.000057 |
| IL36B | 0.000055 |
| ZNF69 | 0.000055 |
| LCE2B | 0.000054 |
| TSHB | 0.000054 |
| SOCS5 | 0.000053 |
| GOLPH3 | 0.000053 |
| DNAJC4 | 0.000052 |
| ZBTB2 | 0.000051 |
| NPAS1 | 0.000048 |
| AKAP11 | 0.000047 |
| ATP7A | 0.000047 |
| GRID1 | 0.000047 |
| MAGIX | 0.000045 |
| MYO5A | 0.000045 |
| ZRSR2 | 0.000045 |
| KLF11 | 0.000045 |
| MED16 | 0.000044 |
| FAM46A | 0.000043 |
| PPP2R5C | 0.000043 |
| GOLGA5 | 0.000043 |
| LPAR4 | 0.000042 |
| PGA5 | 0.000041 |
| TMEM82 | 0.000041 |
| LYZL4 | 0.000040 |
| DCP1A | 0.000040 |
| RASEF | 0.000040 |
| OSTC | 0.000039 |
| SCAND1 | 0.000039 |
| MAD1L1 | 0.000038 |
| SLC26A2 | 0.000037 |
| LGALS7B | 0.000036 |
| FCF1 | 0.000036 |
| MED21 | 0.000036 |
| C5orf52 | 0.000036 |
| DRAP1 | 0.000036 |
| TCF15 | 0.000035 |
| SRP72 | 0.000035 |
| CRBN | 0.000034 |
| MAPK14 | 0.000033 |
| GRK6 | 0.000033 |
| LY6G5C | 0.000033 |
| COQ2 | 0.000031 |
| LAMPS | 0.000031 |
| CHCHD6 | 0.000030 |
| CAMLG | 0.000030 |
| LCE3A | 0.000028 |
| MLLT1 | 0.000027 |
| GIMD1 | 0.000027 |
| SHMT1 | 0.000026 |
| STT3A | 0.000026 |
| NPFF | 0.000025 |
| FAM32A | 0.000023 |
| FAM134A | 0.000023 |
| DEFB113 | 0.000021 |
| KCNQ4 | 0.000021 |
| NEDD8 | 0.000020 |
| TMCO1 | 0.000020 |
| AAMP | 0.000019 |
| SCGB1D4 | 0.000019 |
| AAK1 | 0.000019 |
| ZNF853 | 0.000019 |
| YIPF2 | 0.000018 |
| GTF2H5 | 0.000017 |
| PDP1 | 0.000016 |
| ERC1 | 0.000015 |
| EHF | 0.000014 |
| DMRT2 | 0.000013 |
| SLC16A10 | 0.000012 |
| NUDT19 | 0.000012 |
| SLC12A9 | 0.000009 |
| ZPR1 | 0.000009 |
| RAB3D | 0.000009 |
| MVK | 0.000009 |
| GMPPA | 0.000005 |
| TCEAL1 | 0.000004 |
| ARX | 0.000003 |
| CT45A5 | 0.000003 |
| CT47A12 | 0.000003 |
| ZNHIT2 | 0.000002 |
| BRMS1 | 0.000002 |
| MRPS24 | 0.000001 |
| SMIM23 | 0.000001 |
| SERPINB12 | −0.000001 |
| MLH1 | −0.000001 |
| ANXA13 | −0.000001 |
| IRF6 | −0.000002 |
| PNO1 | −0.000002 |
| DAP3 | −0.000002 |
| RBBP9 | −0.000002 |
| LONRF1 | −0.000003 |
| ADAMTS19 | −0.000003 |
| MRPL33 | −0.000004 |
| SLC30A7 | −0.000004 |
| SPIRE1 | −0.000005 |
| LZTS2 | −0.000005 |
| NUP98 | −0.000005 |
| CCDC174 | −0.000006 |
| KLF16 | −0.000006 |
| CNIH1 | −0.000006 |
| ZMPSTE24 | −0.000007 |
| TEX33 | −0.000007 |
| IFT88 | −0.000007 |
| NOL3 | −0.000007 |
| DAPK3 | −0.000008 |
| PDIA4 | −0.000009 |
| RABI1A | −0.000010 |
| PGRMC2 | −0.000010 |
| TM9SF4 | −0.000010 |
| EBP | −0.000010 |
| PSMD13 | −0.000011 |
| WDR830S | −0.000012 |
| PARK7 | −0.000012 |
| TMEM116 | −0.000013 |
| NDUFA3 | −0.000013 |
| MMP17 | −0.000013 |
| USP17L8 | −0.000014 |
| UBP1 | −0.000015 |
| SLAIN1 | −0.000016 |
| LCE1A | −0.000017 |
| TRIM7 | −0.000017 |
| SAMM50 | −0.000017 |
| FBXO25 | −0.000018 |
| TMEM222 | −0.000018 |
| MTPN | −0.000019 |
| LRRC10B | −0.000019 |
| CT47A6 | −0.000020 |
| RBM3 | −0.000022 |
| SSR1 | −0.000022 |
| ZNF281 | −0.000023 |
| PHAX | −0.000023 |
| GPN3 | −0.000024 |
| TOX4 | −0.000026 |
| PDCD4 | −0.000026 |
| ANKRD55 | −0.000027 |
| PYGO1 | −0.000032 |
| EAF1 | −0.000033 |
| CDPF1 | −0.000033 |
| NME4 | −0.000035 |
| ETFA | −0.000037 |
| CTSF | −0.000041 |
| TRIM65 | −0.000044 |
| ACYP2 | −0.000045 |
| RBCK1 | −0.000046 |
| HCRTR2 | −0.000046 |
| PRPF8 | −0.000047 |
| SLC1A5 | −0.000048 |
| AKR7A2 | −0.000049 |
| KRT24 | −0.000050 |
| YPEL3 | −0.000051 |
| STX10 | −0.000051 |
| RPS26 | −0.000053 |
| NUTM2D | −0.000055 |
| PPP1R2 | −0.000056 |
| CDC42BPB | −0.000057 |
| RNF123 | −0.000057 |
| COX18 | −0.000060 |
| TBL3 | −0.000060 |
| CDX4 | −0.000060 |
| LRRC41 | −0.000060 |
| MUC12 | −0.000061 |
| PPP1R15B | −0.000061 |
| ATAD1 | −0.000061 |
| HHLA3 | −0.000062 |
| AFF1 | −0.000062 |
| AKAP6 | −0.000062 |
| GAB2 | −0.000063 |
| CD276 | −0.000064 |
| SPINT4 | −0.000064 |
| CFAP97 | −0.000064 |
| NDUFB2 | −0.000064 |
| USP27X | −0.000066 |
| ZCRB1 | −0.000067 |
| PURA | −0.000069 |
| LRSAM1 | −0.000070 |
| FAM3A | −0.000070 |
| PLCXD2 | −0.000071 |
| DNAJC1 | −0.000072 |
| AP2A1 | −0.000073 |
| CORO2B | −0.000078 |
| ARSK | −0.000079 |
| USP32 | −0.000080 |
| C3orf62 | −0.000080 |
| INPP5B | −0.000081 |
| ADM | −0.000081 |
| FAM122A | −0.000081 |
| P4HA1 | −0.000082 |
| WDR26 | −0.000083 |
| ANGPTL6 | −0.000084 |
| IL18BP | −0.000085 |
| DDX3X | −0.000085 |
| GRWD1 | −0.000087 |
| RPL7 | −0.000088 |
| EMG1 | −0.000090 |
| SLC25A25 | −0.000091 |
| ANKS6 | −0.000092 |
| OTUD5 | −0.000094 |
| FAM25C | −0.000094 |
| ALG14 | −0.000095 |
| NDST2 | −0.000096 |
| LEMD1 | −0.000096 |
| PLB1 | −0.000097 |
| RTF1 | −0.000098 |
| ILVBL | −0.000098 |
| ZMAT3 | −0.000098 |
| USO1 | −0.000098 |
| NOL6 | −0.000098 |
| MPL | −0.000099 |
| FAM83G | −0.000099 |
| LAMTOR3 | −0.000100 |
| SEC22C | −0.000101 |
| ERCC5 | −0.000101 |
| UBE2Z | −0.000102 |
| TMEM217 | −0.000103 |
| SMARCA5 | −0.000105 |
| IMP4 | −0.000105 |
| FUT2 | −0.000106 |
| ZBTB46 | −0.000107 |
| TMEM123 | −0.000109 |
| DPM3 | −0.000110 |
| TAB2 | −0.000111 |
| CCDC91 | −0.000113 |
| EFCAB6 | −0.000114 |
| ZNF425 | −0.000114 |
| RFX2 | −0.000116 |
| BPNT1 | −0.000116 |
| NCOR1 | −0.000116 |
| MAP3K1 | −0.000117 |
| GNB2 | −0.000118 |
| NENF | −0.000118 |
| NSRP1 | −0.000119 |
| SYK | −0.000120 |
| PSMC2 | −0.000121 |
| SCO1 | −0.000121 |
| GTPBP1 | −0.000121 |
| TMEM185B | −0.000122 |
| ZBED3 | −0.000123 |
| KRT6B | −0.000125 |
| TBC1D3K | −0.000125 |
| SCGB1C1 | −0.000125 |
| DUSP22 | −0.000126 |
| LIPG | −0.000126 |
| LCN1 | −0.000126 |
| PTDSS1 | −0.000126 |
| ALKBH3 | −0.000126 |
| PI4KB | −0.000126 |
| CCDC146 | −0.000127 |
| SPINT2 | −0.000128 |
| FAM149A | −0.000128 |
| SEC24B | −0.000130 |
| RAB6C | −0.000131 |
| RAD21L1 | −0.000131 |
| LSM6 | −0.000131 |
| TMEM184B | −0.000132 |
| PDE8B | −0.000133 |
| F2RL2 | −0.000135 |
| FBXW11 | −0.000135 |
| OR3A1 | −0.000136 |
| TMEM203 | −0.000136 |
| PAPD5 | −0.000137 |
| GAK | −0.000137 |
| R3HCC1 | −0.000138 |
| FBXO15 | −0.000138 |
| OAZ2 | −0.000139 |
| ATP6V1E2 | −0.000139 |
| FER | −0.000139 |
| TRAPPC4 | −0.000140 |
| HSFX2 | −0.000141 |
| MC2R | −0.000142 |
| ARHGAP21 | −0.000143 |
| BRF2 | −0.000143 |
| ATIC | −0.000144 |
| DNAJB1 | −0.000144 |
| OPN1MW | −0.000145 |
| TOMM20 | −0.000146 |
| ATP8B4 | −0.000147 |
| FUT4 | −0.000147 |
| ADRA2A | −0.000148 |
| PEX3 | −0.000148 |
| 43531 | −0.000149 |
| PIGY | −0.000150 |
| KLRF2 | −0.000150 |
| CALM1 | −0.000153 |
| MYO1D | −0.000155 |
| FIGN | −0.000155 |
| CMTM4 | −0.000156 |
| MMGT1 | −0.000157 |
| DPRX | −0.000158 |
| USP43 | −0.000158 |
| MTR | −0.000160 |
| VPS4A | −0.000161 |
| BCAT2 | −0.000161 |
| SIK3 | −0.000161 |
| MYCBP | −0.000162 |
| CDK9 | −0.000162 |
| RSG1 | −0.000162 |
| CCDC81 | −0.000163 |
| PPP1R36 | −0.000163 |
| IST1 | −0.000164 |
| PSMA6 | −0.000164 |
| RER1 | −0.000165 |
| AP1M2 | −0.000166 |
| SLC38A7 | −0.000166 |
| UBE2D1 | −0.000167 |
| ERICH1 | −0.000167 |
| TRIML1 | −0.000170 |
| C12orf10 | −0.000170 |
| ARHGEF2 | −0.000171 |
| CCDC25 | −0.000171 |
| ZNF563 | −0.000171 |
| UPB1 | −0.000171 |
| TNFRSF18 | −0.000172 |
| TBC1D28 | −0.000172 |
| BIN3 | −0.000173 |
| ADAMTS16 | −0.000174 |
| STXBP1 | −0.000175 |
| CDC37L1 | −0.000179 |
| TBC1D17 | −0.000180 |
| UPRT | −0.000182 |
| RAB10 | −0.000183 |
| ODF1 | −0.000184 |
| B3GLCT | −0.000184 |
| RPE65 | −0.000184 |
| ZFPL1 | −0.000184 |
| DDX47 | −0.000185 |
| C19orf54 | −0.000185 |
| TBCEL | −0.000187 |
| GJB6 | −0.000187 |
| PAFAH1B1 | −0.000188 |
| ARSG | −0.000189 |
| COLGALT1 | −0.000192 |
| SRBD1 | −0.000194 |
| PRRC1 | −0.000194 |
| RADIL | −0.000195 |
| CDC42BPG | −0.000195 |
| NID2 | −0.000197 |
| UVRAG | −0.000197 |
| TESK1 | −0.000199 |
| PKN3 | −0.000199 |
| FZD1 | −0.000200 |
| ADGRG1 | −0.000201 |
| RBMY1F | −0.000202 |
| OCIAD1 | −0.000202 |
| RAB4A | −0.000203 |
| UBA52 | −0.000203 |
| SLC26A3 | −0.000204 |
| CLTA | −0.000205 |
| OPN1MW2 | −0.000205 |
| SPA17 | −0.000205 |
| MAP3K14 | −0.000205 |
| KIAA0825 | −0.000206 |
| NF2 | −0.000207 |
| RASA4B | −0.000207 |
| ANKRD34C | −0.000208 |
| GNAI1 | −0.000209 |
| CACNG1 | −0.000210 |
| NDUFB9 | −0.000210 |
| TAB1 | −0.000211 |
| AVEN | −0.000212 |
| SCN10A | −0.000212 |
| RIMBP3 | −0.000212 |
| C3orf84 | −0.000213 |
| MRPL45 | −0.000213 |
| FFAR1 | −0.000216 |
| RFXANK | −0.000216 |
| C3orf80 | −0.000217 |
| PGM2 | −0.000217 |
| UBE2F | −0.000218 |
| PITPNC1 | −0.000218 |
| ABCF2 | −0.000219 |
| HECTD3 | −0.000219 |
| OPN1LW | −0.000219 |
| FAM126A | −0.000221 |
| PTHLH | −0.000221 |
| ZDHHC4 | −0.000221 |
| IDI2 | −0.000221 |
| BBC3 | −0.000224 |
| PAD12 | −0.000224 |
| DPP8 | −0.000225 |
| DGKQ | −0.000225 |
| E2F4 | −0.000225 |
| RNF170 | −0.000227 |
| OMA1 | −0.000229 |
| ABHD13 | −0.000230 |
| LPCAT4 | −0.000233 |
| B3GALNT1 | −0.000233 |
| ANKMY2 | −0.000234 |
| DEFB131 | −0.000234 |
| SEC22B | −0.000234 |
| EP300 | −0.000235 |
| PIK3CB | −0.000235 |
| HERC3 | −0.000236 |
| PEX16 | −0.000237 |
| SSU72 | −0.000237 |
| FAM151B | −0.000237 |
| CCDC102B | −0.000237 |
| PDE8A | −0.000238 |
| BMPR1A | −0.000240 |
| RBM7 | −0.000240 |
| FN3K | −0.000240 |
| VPS35 | −0.000241 |
| IKZF2 | −0.000241 |
| RMDN1 | −0.000241 |
| DUPD1 | −0.000242 |
| UACA | −0.000242 |
| NBEAL2 | −0.000242 |
| BET1 | −0.000243 |
| FAM170B | −0.000243 |
| URM1 | −0.000243 |
| CRCT1 | −0.000244 |
| LAMTOR5 | −0.000245 |
| GLRB | −0.000245 |
| RPP25L | −0.000247 |
| GOLGA4 | −0.000249 |
| GUCY1B3 | −0.000250 |
| TP53RK | −0.000250 |
| SLC25A4 | −0.000251 |
| UBE2J1 | −0.000251 |
| ZNF592 | −0.000251 |
| FBXO18 | −0.000251 |
| PTGFRN | −0.000252 |
| KLLN | −0.000252 |
| HMGCR | −0.000252 |
| METTL13 | −0.000252 |
| DLD | −0.000253 |
| SCFD2 | −0.000253 |
| COX7A2L | −0.000253 |
| ATP6V1G1 | −0.000253 |
| DEDD | −0.000255 |
| DHX38 | −0.000260 |
| CCDC94 | −0.000261 |
| SPTLC1 | −0.000262 |
| DHX29 | −0.000263 |
| RIMS1 | −0.000263 |
| MTFMT | −0.000264 |
| WDR44 | −0.000264 |
| ATP5E | −0.000267 |
| PGA4 | −0.000268 |
| ZNF32 | −0.000268 |
| RYR2 | −0.000270 |
| CIAPIN1 | −0.000270 |
| TBC1D3G | −0.000270 |
| SPINT3 | −0.000270 |
| TCF4 | −0.000271 |
| PDP2 | −0.000271 |
| RPS27A | −0.000272 |
| COMMD7 | −0.000273 |
| WFDC9 | −0.000273 |
| FAM69A | −0.000274 |
| STYX | −0.000274 |
| HMCN2 | −0.000274 |
| SPANXA1 | −0.000275 |
| ZNF454 | −0.000276 |
| PMF1 | −0.000276 |
| CXCL13 | −0.000277 |
| C1orf43 | −0.000281 |
| CASP9 | −0.000282 |
| COL28A1 | −0.000283 |
| GCA | −0.000283 |
| CLDND2 | −0.000285 |
| 1TM2C | −0.000286 |
| TOMM40L | −0.000289 |
| IDE | −0.000290 |
| RRP7A | −0.000291 |
| ZNF552 | −0.000291 |
| CRH | −0.000292 |
| SPATA19 | −0.000292 |
| TMEM176A | −0.000292 |
| SMPD2 | −0.000295 |
| CABP5 | −0.000295 |
| RNF7 | −0.000295 |
| ADPRM | −0.000296 |
| NUTF2 | −0.000296 |
| ITPK1 | −0.000297 |
| L3MBTL2 | −0.000298 |
| CYP2J2 | −0.000298 |
| BAP1 | −0.000300 |
| GRIIL2 | −0.000300 |
| ADAMTS5 | −0.000301 |
| TRIM35 | −0.000301 |
| MYH8 | −0.000301 |
| PDE1A | −0.000301 |
| DNAH7 | −0.000302 |
| TRIM26 | −0.000303 |
| ADA | −0.000304 |
| TCHH | −0.000305 |
| PSMA8 | −0.000305 |
| GAPVD1 | −0.000307 |
| LARP4B | −0.000308 |
| WBP4 | −0.000308 |
| ATP5SL | −0.000308 |
| DDX24 | −0.000309 |
| TNRC18 | −0.000310 |
| MRPL49 | −0.000312 |
| EIF1B | −0.000313 |
| VASH1 | −0.000315 |
| PNLIPRP2 | −0.000316 |
| ANKRD35 | −0.000317 |
| DESI2 | −0.000318 |
| ALKBH5 | −0.000320 |
| GRHPR | −0.000320 |
| MRPL23 | −0.000321 |
| TRPC4AP | −0.000321 |
| FBX038 | −0.000321 |
| STC1 | −0.000323 |
| SPANXC | −0.000323 |
| CLEC16A | −0.000324 |
| SNX27 | −0.000324 |
| FAM214A | −0.000326 |
| MT1B | −0.000330 |
| PRKAG2 | −0.000331 |
| PAOX | −0.000332 |
| SH3PXD2B | −0.000332 |
| CNEP1R1 | −0.000332 |
| SULT1A3 | −0.000332 |
| SPG11 | −0.000332 |
| ZNF608 | −0.000334 |
| IFNA1 | −0.000334 |
| SEC14L1 | −0.000334 |
| EIF3H | −0.000335 |
| PRR16 | −0.000337 |
| DDR1 | −0.000337 |
| PRKAA1 | −0.000337 |
| SLC30A5 | −0.000338 |
| SPANXA2 | −0.000339 |
| SMIM15 | −0.000339 |
| CHMP7 | −0.000340 |
| FAM58A | −0.000342 |
| RNF41 | −0.000343 |
| REEP4 | −0.000343 |
| AP2M1 | −0.000344 |
| SOCS6 | −0.000345 |
| HIPK1 | −0.000345 |
| ART3 | −0.000347 |
| PDIA6 | −0.000347 |
| ALKBH7 | −0.000347 |
| CALM2 | −0.000348 |
| ZFYVE1 | −0.000349 |
| SLC35A3 | −0.000350 |
| SLC25A37 | −0.000350 |
| SULT1E1 | −0.000350 |
| CTNND2 | −0.000350 |
| RRP12 | −0.000350 |
| TM9SF3 | −0.000352 |
| ANKRD28 | −0.000353 |
| BRDT | −0.000354 |
| ISOC1 | −0.000355 |
| RPS2 | −0.000358 |
| SMURF2 | −0.000358 |
| WHAMM | −0.000359 |
| TBC1D3F | −0.000359 |
| THEM4 | −0.000360 |
| AEN | −0.000361 |
| NDUFS4 | −0.000361 |
| TP53INP2 | −0.000362 |
| HIGD1A | −0.000362 |
| HAND1 | −0.000362 |
| GAR1 | −0.000363 |
| TIMMDC1 | −0.000363 |
| TRIM69 | −0.000363 |
| RNF26 | −0.000363 |
| AEBP2 | −0.000364 |
| UBE2D3 | −0.000364 |
| FUT1 | −0.000365 |
| RPL23A | −0.000365 |
| KLK9 | −0.000365 |
| OPA3 | −0.000366 |
| RPUSD3 | −0.000366 |
| SRA1 | −0.000367 |
| IFNLR1 | −0.000368 |
| NBAS | −0.000368 |
| ZFYVE26 | −0.000368 |
| COBL | −0.000369 |
| MMP3 | −0.000369 |
| SMCR8 | −0.000370 |
| SLC34A1 | −0.000371 |
| RNASE13 | −0.000371 |
| UQCR11 | −0.000371 |
| PLS1 | −0.000372 |
| NUDT12 | −0.000372 |
| SNX5 | −0.000372 |
| MDH2 | −0.000373 |
| MB21D2 | −0.000373 |
| C4orf33 | −0.000377 |
| TUBA1C | −0.000377 |
| GKN1 | −0.000377 |
| RPL24 | −0.000377 |
| EPHA4 | −0.000378 |
| XRCC4 | −0.000378 |
| MMD | −0.000379 |
| PAPD4 | −0.000379 |
| HMGCS1 | −0.000380 |
| HINT3 | −0.000383 |
| EIF3G | −0.000384 |
| PPP2R2A | −0.000384 |
| ASNA1 | −0.000384 |
| C4orf47 | −0.000386 |
| TPM1 | −0.000386 |
| TMEM11 | −0.000387 |
| OR2A1 | −0.000388 |
| RPS5 | −0.000388 |
| SLC2A13 | −0.000388 |
| FA2H | −0.000388 |
| FAM175B | −0.000388 |
| C16orf78 | −0.000388 |
| BCL2L2 | −0.000389 |
| CCDC155 | −0.000389 |
| MYLIP | −0.000389 |
| MYL1 | −0.000389 |
| CPEB1 | −0.000390 |
| GPR18 | −0.000390 |
| GAUNT13 | −0.000392 |
| AATF | −0.000392 |
| GOLPH3L | −0.000393 |
| FBRS | −0.000393 |
| MT-ND2 | −0.000396 |
| RTFDC1 | −0.000396 |
| ALKBH8 | −0.000397 |
| ATG2A | −0.000397 |
| TINF2 | −0.000398 |
| CPB1 | −0.000401 |
| BPGM | −0.000401 |
| DDX21 | −0.000401 |
| TAF3 | −0.000401 |
| OR6N1 | −0.000402 |
| ATG9A | −0.000403 |
| PPP2R1A | −0.000403 |
| SUPT6H | −0.000404 |
| SNX29 | −0.000405 |
| FAM160A1 | −0.000405 |
| RPLPO | −0.000406 |
| ANXA10 | −0.000406 |
| WASL | −0.000407 |
| RPN1 | −0.000407 |
| C19orf53 | −0.000409 |
| PLEKHH2 | −0.000410 |
| TIMM22 | −0.000410 |
| CD300LD | −0.000411 |
| MLF2 | −0.000413 |
| KLHL18 | −0.000413 |
| PNLIPRP3 | −0.000414 |
| MAP1S | −0.000415 |
| PPP1R10 | −0.000416 |
| MAX | −0.000417 |
| CLCN1 | −0.000417 |
| PXDN | −0.000417 |
| NT5C2 | −0.000417 |
| TRIML2 | −0.000417 |
| NME3 | −0.000418 |
| SSNA1 | −0.000418 |
| TBC1D25 | −0.000419 |
| MRPL28 | −0.000419 |
| RRS1 | −0.000419 |
| RSPH3 | −0.000420 |
| IWS1 | −0.000420 |
| TAOK2 | −0.000420 |
| RPL41 | −0.000421 |
| TMEM165 | −0.000421 |
| DNAJC8 | −0.000421 |
| EI24 | −0.000422 |
| EHHADH | −0.000424 |
| UTP14C | −0.000424 |
| PLD5 | −0.000424 |
| TMED2 | −0.000424 |
| C20orf141 | −0.000425 |
| RBMXL1 | −0.000425 |
| PRKAG1 | −0.000426 |
| RNF225 | −0.000427 |
| RPLI8A | −0.000428 |
| NICN1 | −0.000428 |
| EXOC8 | −0.000429 |
| TUBGCP2 | −0.000430 |
| DNM2 | −0.000430 |
| CERKL | −0.000431 |
| CANX | −0.000432 |
| CRYBB2 | −0.000432 |
| CKLF | −0.000433 |
| TIIAP4 | −0.000434 |
| LRFN1 | −0.000434 |
| HOXA7 | −0.000435 |
| MROH2A | −0.000435 |
| C6orf89 | −0.000435 |
| TG | −0.000436 |
| VPS51 | −0.000436 |
| TMEM114 | −0.000437 |
| NKX3-1 | −0.000438 |
| CBR3 | −0.000440 |
| TSTA3 | −0.000440 |
| INPP4B | −0.000441 |
| CD164 | −0.000441 |
| HABP2 | −0.000441 |
| LCE3D | −0.000443 |
| PGGT1B | −0.000444 |
| NBN | −0.000444 |
| PRKX | −0.000445 |
| DIO1 | −0.000445 |
| EDDM3B | −0.000449 |
| GCKR | −0.000450 |
| CDH26 | −0.000451 |
| HCCS | −0.000451 |
| USP51 | −0.000451 |
| DUSP11 | −0.000454 |
| COX15 | −0.000454 |
| ALOXE3 | −0.000455 |
| FAM134C | −0.000455 |
| BZW1 | −0.000456 |
| USP38 | −0.000458 |
| METAP2 | −0.000458 |
| CCDC22 | −0.000458 |
| FAM13A | −0.000460 |
| EEF2 | −0.000461 |
| FBX034 | −0.000461 |
| PTGDR2 | −0.000462 |
| RPL37A | −0.000462 |
| ZNF394 | −0.000462 |
| NGLY1 | −0.000463 |
| ATP5F1 | −0.000464 |
| EAPP | −0.000464 |
| FADD | −0.000464 |
| MYH4 | −0.000464 |
| SPTY2D1 | −0.000465 |
| SLC36A2 | −0.000466 |
| SPESP1 | −0.000466 |
| OTUD7B | −0.000466 |
| SAV1 | −0.000468 |
| CIDEA | −0.000470 |
| SPATA20 | −0.000470 |
| MATN4 | −0.000471 |
| TMEM106B | −0.000475 |
| HELZ2 | −0.000475 |
| SYVN1 | −0.000476 |
| DHRS7 | −0.000477 |
| SLC39A2 | −0.000477 |
| RNFT1 | −0.000477 |
| ADSS | −0.000478 |
| MEPCE | −0.000478 |
| TXNDC9 | −0.000478 |
| ADAMTSL2 | −0.000479 |
| ALG3 | −0.000479 |
| OTUB1 | −0.000479 |
| TMEM205 | −0.000479 |
| KRTCAP2 | −0.000481 |
| MOGS | −0.000481 |
| MYO18A | −0.000481 |
| RCL1 | −0.000481 |
| SMIM19 | −0.000481 |
| IAH1 | −0.000482 |
| LIPK | −0.000483 |
| 43532 | −0.000485 |
| UBXN6 | −0.000487 |
| VAMP7 | −0.000489 |
| PPA2 | −0.000489 |
| LCN10 | −0.000489 |
| ZNF267 | −0.000491 |
| KRT76 | −0.000491 |
| NCLN | −0.000492 |
| BTBD16 | −0.000493 |
| LRRC74A | −0.000493 |
| FSHR | −0.000493 |
| TMEM19 | −0.000495 |
| UFSP1 | −0.000497 |
| NEK10 | −0.000497 |
| ATP6V1H | −0.000498 |
| MXI1 | −0.000498 |
| SZRD1 | −0.000499 |
| CACNA1F | −0.000499 |
| MT-ND4 | −0.000500 |
| ROM1 | −0.000502 |
| DPEP1 | −0.000502 |
| VDAC1 | −0.000504 |
| PEX2 | −0.000507 |
| MMADHC | −0.000509 |
| BAX | −0.000511 |
| NDUFA2 | −0.000511 |
| GPR78 | −0.000511 |
| KCNJ18 | −0.000512 |
| SUGCT | −0.000515 |
| MTMR12 | −0.000515 |
| S1PR3 | −0.000516 |
| FAM127C | −0.000516 |
| EIF2D | −0.000516 |
| HTT | −0.000516 |
| CSNK2A2 | −0.000520 |
| SOX8 | −0.000520 |
| MYPN | −0.000521 |
| MSX1 | −0.000523 |
| GUCY2F | −0.000523 |
| UBXN8 | −0.000524 |
| MAGI1 | −0.000524 |
| AGPAT3 | −0.000525 |
| SLC25A5 | −0.000525 |
| RASSF1 | −0.000528 |
| TOPI | −0.000530 |
| CHCHD10 | −0.000530 |
| SDF4 | −0.000531 |
| POMGNT2 | −0.000532 |
| MYCBP2 | −0.000532 |
| ARRDC1 | −0.000533 |
| SDE2 | −0.000534 |
| SPATA2 | −0.000538 |
| TMEM33 | −0.000539 |
| BLZF1 | −0.000539 |
| SPINK14 | −0.000539 |
| EPN1 | −0.000540 |
| RALA | −0.000540 |
| FKBP7 | −0.000541 |
| SHROOM2 | −0.000541 |
| COG7 | −0.000542 |
| ERMN | −0.000542 |
| PHB | −0.000543 |
| GSTA4 | −0.000543 |
| PIGS | −0.000544 |
| POLR2M | −0.000544 |
| PCIF1 | −0.000545 |
| PF4V1 | −0.000546 |
| TWISTNB | −0.000546 |
| MSC | −0.000547 |
| PHB2 | −0.000547 |
| CRACR2B | −0.000548 |
| TXNDC15 | −0.000548 |
| ADAM10 | −0.000551 |
| ZC3H7B | −0.000552 |
| PLAGL2 | −0.000553 |
| SSR4 | −0.000553 |
| PLGRKT | −0.000554 |
| SUSD4 | −0.000555 |
| IARS2 | −0.000555 |
| ANKRD31 | −0.000556 |
| HMG20A | −0.000556 |
| PKD2 | −0.000556 |
| SMIM20 | −0.000558 |
| RPS13 | −0.000558 |
| STARD5 | −0.000559 |
| FAM160B1 | −0.000560 |
| ZC3HAV1 | −0.000560 |
| ACER1 | −0.000561 |
| GPATCH4 | −0.000562 |
| LSR | −0.000563 |
| TXNDC12 | −0.000563 |
| APLF | −0.000565 |
| MANBAL | −0.000565 |
| TMC1 | −0.000566 |
| MVB12A | −0.000567 |
| RRP9 | −0.000567 |
| C11orf63 | −0.000568 |
| EIF4B | −0.000568 |
| SH3GLB1 | −0.000568 |
| CDH6 | −0.000568 |
| ARFIP1 | −0.000569 |
| C19orf24 | −0.000569 |
| TSPAN10 | −0.000569 |
| KLHL33 | −0.000570 |
| SLC16A9 | −0.000571 |
| TSG101 | −0.000571 |
| RASGEF1B | −0.000573 |
| PSMD9 | −0.000575 |
| CCDC71L | −0.000575 |
| CYB5D1 | −0.000577 |
| TNFAIP6 | −0.000577 |
| PPP6C | −0.000577 |
| GALNT1 | −0.000578 |
| RABGGTA | −0.000578 |
| SORBS2 | −0.000578 |
| CNOT8 | −0.000578 |
| SDHC | −0.000578 |
| PGLYRP3 | −0.000579 |
| UBE2Q2 | −0.000580 |
| TXN | −0.000581 |
| VCP | −0.000581 |
| SRY | −0.000582 |
| HEPN1 | −0.000583 |
| PRR5 | −0.000583 |
| EQTN | −0.000583 |
| LAMTOR4 | −0.000584 |
| PCMTD1 | −0.000587 |
| IL11RA | −0.000588 |
| DNAJB14 | −0.000588 |
| SLC25A28 | −0.000589 |
| IMPACT | −0.000591 |
| SP2 | −0.000592 |
| SEMA6A | −0.000592 |
| SNAP29 | −0.000593 |
| ZMAT2 | −0.000593 |
| ACOT13 | −0.000593 |
| SLC9A3R1 | −0.000593 |
| ARL4A | −0.000593 |
| GBE1 | −0.000594 |
| PTPN18 | −0.000595 |
| COL16A1 | −0.000595 |
| CKAP4 | −0.000595 |
| PABPC1 | −0.000596 |
| SHQ1 | −0.000596 |
| EIF3A | −0.000599 |
| DMRTC1 | −0.000599 |
| DERL1 | −0.000600 |
| PVR | −0.000600 |
| RNF11 | −0.000601 |
| GDI2 | −0.000602 |
| SNAPC2 | −0.000604 |
| GUSB | −0.000604 |
| RTP5 | −0.000605 |
| PYROXD1 | −0.000607 |
| ZNF208 | −0.000610 |
| ACBD5 | −0.000610 |
| TSSC4 | −0.000611 |
| MFHAS1 | −0.000612 |
| CDC14A | −0.000612 |
| PYURF | −0.000612 |
| SOX5 | −0.000613 |
| SSR3 | −0.000614 |
| FUT10 | −0.000615 |
| AMZ1 | −0.000615 |
| SLC25A44 | −0.000617 |
| DDX19A | −0.000620 |
| RNF6 | −0.000621 |
| GRAMD1A | −0.000621 |
| AP3S1 | −0.000621 |
| GSK3A | −0.000622 |
| CARD17 | −0.000622 |
| RTN4RL2 | −0.000623 |
| ELL | −0.000623 |
| OR5P2 | −0.000623 |
| ABR | −0.000623 |
| RPL18 | −0.000625 |
| TAF12 | −0.000626 |
| SCAP | −0.000627 |
| TOMM6 | −0.000627 |
| MON1B | −0.000628 |
| GALNS | −0.000628 |
| MMAB | −0.000629 |
| MPND | −0.000629 |
| SLC25A3 | −0.000629 |
| MFAP2 | −0.000629 |
| TTC27 | −0.000630 |
| FAM175A | −0.000631 |
| METTL21B | −0.000631 |
| DNTTIP2 | −0.000631 |
| HIST1H4K | −0.000631 |
| BEND2 | −0.000632 |
| ASGR1 | −0.000632 |
| RBX1 | −0.000632 |
| RNF212B | −0.000634 |
| GPR82 | −0.000635 |
| RFPL4AL1 | −0.000635 |
| C20orf27 | −0.000636 |
| CXCL6 | −0.000636 |
| KRT72 | −0.000637 |
| DNAL4 | −0.000637 |
| CRTC2 | −0.000637 |
| TMED10 | −0.000637 |
| TBC1D4 | −0.000637 |
| ZNF671 | −0.000638 |
| UBE2NL | −0.000638 |
| LRRTM4 | −0.000638 |
| EML1 | −0.000639 |
| DPPA3 | −0.000639 |
| CHODL | −0.000639 |
| HSPA4 | −0.000640 |
| FAM90A1 | −0.000640 |
| WDTC1 | −0.000640 |
| ST8SIA4 | −0.000641 |
| ARFGAP2 | −0.000641 |
| GRAMD4 | −0.000641 |
| SLC2A4RG | −0.000641 |
| RPUSD2 | −0.000643 |
| GALK2 | −0.000643 |
| CNPY3 | −0.000644 |
| MAST3 | −0.000644 |
| TFEB | −0.000644 |
| CLPX | −0.000645 |
| SF3B5 | −0.000646 |
| MMP27 | −0.000646 |
| C16orf58 | −0.000647 |
| RUFY1 | −0.000647 |
| FMNL3 | −0.000649 |
| NMT1 | −0.000652 |
| GLYR1 | −0.000652 |
| MESDC2 | −0.000653 |
| CTIF | −0.000653 |
| BACH1 | −0.000653 |
| SMIM12 | −0.000655 |
| TMEM9 | −0.000656 |
| ATL2 | −0.000656 |
| FAM159A | −0.000657 |
| CCDC186 | −0.000658 |
| PSMF1 | −0.000658 |
| CCDC9 | −0.000659 |
| SETDB2 | −0.000659 |
| CD99L2 | −0.000659 |
| PATL2 | −0.000660 |
| PIP5K1C | −0.000660 |
| ENPP7 | −0.000663 |
| GIP | −0.000664 |
| USP8 | −0.000664 |
| RPL30 | −0.000666 |
| DSG3 | −0.000666 |
| CYSLTR2 | −0.000667 |
| CGB8 | −0.000669 |
| GABARAPL2 | −0.000671 |
| RAB18 | −0.000671 |
| KY | −0.000672 |
| RPS29 | −0.000673 |
| IQCK | −0.000674 |
| UBA3 | −0.000675 |
| FAM35A | −0.000675 |
| ULBP2 | −0.000675 |
| SLU7 | −0.000676 |
| UQCRC2 | −0.000678 |
| CCL28 | −0.000678 |
| SLC25A38 | −0.000678 |
| POU1F1 | −0.000679 |
| RBM42 | −0.000679 |
| FAM98A | −0.000679 |
| CAPN5 | −0.000680 |
| SEC24C | −0.000682 |
| IDH3A | −0.000682 |
| RAB12 | −0.000683 |
| BCR | −0.000683 |
| TRAFD1 | −0.000684 |
| PGLYRP4 | −0.000685 |
| ERN1 | −0.000689 |
| UBE2U | −0.000690 |
| CHRNE | −0.000692 |
| RORB | −0.000693 |
| EDF1 | −0.000695 |
| SGK3 | −0.000695 |
| AP2B1 | −0.000696 |
| RPS10 | −0.000696 |
| DPPA4 | −0.000696 |
| DCXR | −0.000698 |
| LOXL1 | −0.000699 |
| TRPC4 | −0.000700 |
| CWF19L2 | −0.000701 |
| C15orf53 | −0.000703 |
| MATN2 | −0.000704 |
| SDF2 | −0.000705 |
| RPL5 | −0.000706 |
| SFR1 | −0.000708 |
| BCAS4 | −0.000708 |
| TRIM2 | −0.000709 |
| CTTN | −0.000709 |
| IMPDH2 | −0.000710 |
| UNC45A | −0.000712 |
| PTRHD1 | −0.000714 |
| SYAP1 | −0.000716 |
| EXT2 | −0.000717 |
| HYAL4 | −0.000717 |
| ABI1 | −0.000719 |
| MOB2 | −0.000719 |
| LRRC38 | −0.000719 |
| COPG1 | −0.000720 |
| SLC39A7 | −0.000720 |
| LSAMP | −0.000722 |
| GPATCH3 | −0.000722 |
| CDH23 | −0.000723 |
| MAP2K4 | −0.000723 |
| WBP11 | −0.000723 |
| DBNL | −0.000724 |
| CTLA4 | −0.000724 |
| WDR82 | −0.000725 |
| ARHGEF1 | −0.000725 |
| MCCC2 | −0.000726 |
| RPL35 | −0.000728 |
| RAB8A | −0.000729 |
| FXN | −0.000730 |
| HTR2B | −0.000731 |
| TEX2 | −0.000731 |
| PPARA | −0.000732 |
| KCNN4 | −0.000732 |
| AIF1L | −0.000732 |
| CRYBA4 | −0.000732 |
| TMEM72 | −0.000733 |
| ROCK2 | −0.000733 |
| AP1M1 | −0.000733 |
| TBC1D13 | −0.000736 |
| NFKBIB | −0.000737 |
| KIAA0141 | −0.000737 |
| OR10A3 | −0.000737 |
| KLF7 | −0.000738 |
| SYCN | −0.000738 |
| INSIG1 | −0.000739 |
| GNAL | −0.000741 |
| ERLIN2 | −0.000742 |
| POLDIP3 | −0.000742 |
| ZHX3 | −0.000743 |
| TRPM6 | −0.000744 |
| FAM86B2 | −0.000744 |
| MLYCD | −0.000744 |
| C9orf3 | −0.000746 |
| PDLIM5 | −0.000746 |
| FLYWCH2 | −0.000746 |
| CCDC149 | −0.000747 |
| CATSPERB | −0.000747 |
| TSPAN6 | −0.000749 |
| PTPN23 | −0.000749 |
| ARHGEF4 | −0.000749 |
| SLC25A35 | −0.000750 |
| ADRM1 | −0.000750 |
| APOA1 | −0.000751 |
| RIC8A | −0.000751 |
| CSNK1A1 | −0.000753 |
| PPP1R13B | −0.000753 |
| ADH4 | −0.000754 |
| SKP1 | −0.000754 |
| ARHGEF37 | −0.000755 |
| SC5D | −0.000756 |
| PEX14 | −0.000757 |
| TRAM2 | −0.000757 |
| VPS53 | −0.000758 |
| SRP68 | −0.000758 |
| PRELID1 | −0.000759 |
| ZNF655 | −0.000759 |
| FAM53B | −0.000760 |
| SLC14A2 | −0.000761 |
| XRCC6 | −0.000763 |
| S100A7 | −0.000763 |
| PBX2 | −0.000763 |
| ENTHD1 | −0.000764 |
| DDAH2 | −0.000764 |
| NCK1 | −0.000766 |
| HSF5 | −0.000766 |
| DEFB1 | −0.000766 |
| IKBKG | −0.000768 |
| RIPK1 | −0.000768 |
| MARVELD2 | −0.000769 |
| ZNF331 | −0.000769 |
| LUZP2 | −0.000771 |
| B9D2 | −0.000774 |
| OCM | −0.000775 |
| MYO6 | −0.000777 |
| MRPS18A | −0.000778 |
| SLC35F5 | −0.000779 |
| ENPEP | −0.000780 |
| GPR107 | −0.000781 |
| TDRD3 | −0.000782 |
| GALNT2 | −0.000783 |
| PKM | −0.000783 |
| PHYH | −0.000783 |
| DUS2 | −0.000783 |
| DGKA | −0.000785 |
| COMMD9 | −0.000789 |
| SND1 | −0.000790 |
| SEC23A | −0.000794 |
| HSPA1L | −0.000794 |
| SPRR2G | −0.000794 |
| ADAMTS7 | −0.000796 |
| EEA1 | −0.000796 |
| SUN2 | −0.000796 |
| DCTN6 | −0.000797 |
| ACRBP | −0.000799 |
| OSBPL9 | −0.000800 |
| IFNAR1 | −0.000801 |
| NSA2 | −0.000801 |
| EIF3F | −0.000802 |
| MFAP1 | −0.000802 |
| KCTD2 | −0.000802 |
| TANC2 | −0.000803 |
| LIMK2 | −0.000803 |
| CCDC114 | −0.000804 |
| PRR33 | −0.000804 |
| RPS15A | −0.000804 |
| SLC35E1 | −0.000808 |
| CIISY1 | −0.000809 |
| IQUB | −0.000809 |
| OR3A2 | −0.000810 |
| COPZ1 | −0.000810 |
| PRKAR1B | −0.000811 |
| TMEM104 | −0.000814 |
| PAFAH1B2 | −0.000815 |
| SUM03 | −0.000815 |
| KLHL10 | −0.000815 |
| ANKFY1 | −0.000816 |
| CCR9 | −0.000818 |
| THGIL | −0.000819 |
| NACA | −0.000819 |
| COL4A1 | −0.000820 |
| SDHB | −0.000820 |
| PGLS | −0.000821 |
| PGPEP1L | −0.000822 |
| LMAN2L | −0.000823 |
| DYRK3 | −0.000824 |
| EIF4A1 | −0.000825 |
| CDK7 | −0.000825 |
| RAB35 | −0.000828 |
| NTRK1 | −0.000828 |
| DYNLRB1 | −0.000829 |
| FAM25A | −0.000830 |
| CHRAC1 | −0.000832 |
| PDHB | −0.000832 |
| CDC42SE1 | −0.000833 |
| ETV4 | −0.000834 |
| TPRA1 | −0.000836 |
| SF3B4 | −0.000838 |
| 43717 | −0.000839 |
| ACSBG1 | −0.000839 |
| COX7C | −0.000839 |
| PPP4C | −0.000839 |
| TMEM167A | −0.000839 |
| NFAT5 | −0.000840 |
| MRFAP1 | −0.000840 |
| C9orf16 | −0.000841 |
| CDADC1 | −0.000841 |
| GOLIM4 | −0.000843 |
| UBAC2 | −0.000844 |
| DDX54 | −0.000848 |
| NRN1L | −0.000849 |
| USP19 | −0.000849 |
| DDRGK1 | −0.000850 |
| S100G | −0.000850 |
| TMCO5A | −0.000850 |
| CBLN3 | −0.000851 |
| LCE2A | −0.000851 |
| CUBN | −0.000853 |
| IKBIP | −0.000853 |
| PRCD | −0.000854 |
| EP6K1 | −0.000854 |
| VPS28 | −0.000854 |
| STOX2 | −0.000855 |
| C6orf47 | −0.000855 |
| TRMT1L | −0.000855 |
| USP2 | −0.000857 |
| VPS36 | −0.000857 |
| DHCR7 | −0.000858 |
| OR2B11 | −0.000859 |
| MPRIP | −0.000859 |
| TMEM147 | −0.000861 |
| ADPRHL2 | −0.000861 |
| RSL1D1 | −0.000861 |
| ZNF277 | −0.000863 |
| SAR1B | −0.000864 |
| RNF17 | −0.000865 |
| PSMB3 | −0.000865 |
| FER1L6 | −0.000867 |
| GTF2B | −0.000867 |
| NIPAL3 | −0.000868 |
| MT-ND3 | −0.000869 |
| MYO1A | −0.000869 |
| OR5P3 | −0.000869 |
| CGB5 | −0.000869 |
| MRPL16 | −0.000869 |
| ZNHIT1 | −0.000872 |
| PDZD8 | −0.000872 |
| USP40 | −0.000872 |
| RGMA | −0.000872 |
| SLC10A7 | −0.000873 |
| TREX1 | −0.000875 |
| PEX7 | −0.000875 |
| GRAMD1C | −0.000875 |
| FAM8A1 | −0.000876 |
| RBP7 | −0.000878 |
| C16orf86 | −0.000879 |
| FLRT2 | −0.000879 |
| RBSN | −0.000882 |
| OR2W3 | −0.000884 |
| TRMT112 | −0.000888 |
| ZNF469 | −0.000888 |
| OR5G3 | −0.000889 |
| EIF3E | −0.000890 |
| CYP7A1 | −0.000891 |
| MRGPRX2 | −0.000892 |
| TMEM87B | −0.000893 |
| TEN1 | −0.000894 |
| PDE12 | −0.000895 |
| CTNNBL1 | −0.000896 |
| STT3B | −0.000897 |
| RGS14 | −0.000898 |
| PCDH10 | −0.000898 |
| UBE2B | −0.000899 |
| PSMA5 | −0.000899 |
| ADI1 | −0.000899 |
| GPR150 | −0.000901 |
| FAM86C1 | −0.000902 |
| RCN3 | −0.000904 |
| FRMD4A | −0.000905 |
| ITSN2 | −0.000906 |
| CYP4A22 | −0.000907 |
| TSPAN33 | −0.000907 |
| CHID1 | −0.000910 |
| IRF9 | −0.000910 |
| RNF213 | −0.000912 |
| TMEM41B | −0.000912 |
| CCSER2 | −0.000912 |
| RABEPK | −0.000913 |
| ABCB5 | −0.000914 |
| MSMP | −0.000915 |
| DNAJB2 | −0.000915 |
| UBR1 | −0.000916 |
| DNPH1 | −0.000916 |
| KIF9 | −0.000916 |
| KAT5 | −0.000917 |
| MRPL10 | −0.000917 |
| RAB11FIP2 | −0.000919 |
| TFRC | −0.000919 |
| PES1 | −0.000923 |
| NRAP | −0.000924 |
| NDUFC1 | −0.000926 |
| HTR1D | −0.000927 |
| CRISPLD1 | −0.000928 |
| PPP1CB | −0.000929 |
| TCF7 | −0.000931 |
| CLTC | −0.000931 |
| CCDC51 | −0.000932 |
| ATP6V1G3 | −0.000932 |
| PTPRF | −0.000933 |
| ZNF622 | −0.000933 |
| ASCC1 | −0.000935 |
| SMIM4 | −0.000935 |
| EMMP2L | −0.000937 |
| MAPKAP1 | −0.000938 |
| DPF3 | −0.000939 |
| SH3BGRL2 | −0.000940 |
| HSCB | −0.000940 |
| SLC8A1 | −0.000941 |
| BCL7C | −0.000941 |
| STX3 | −0.000941 |
| NDFIP1 | −0.000942 |
| DUSP14 | −0.000943 |
| SYTL2 | −0.000944 |
| AGPS | −0.000945 |
| VKORC1 | −0.000945 |
| SNX11 | −0.000946 |
| ZNF311 | −0.000947 |
| NTS | −0.000948 |
| NFATC2 | −0.000948 |
| SNX14 | −0.000950 |
| MAP3K5 | −0.000951 |
| ATP6V1B2 | −0.000953 |
| ABHD2 | −0.000953 |
| GLS | −0.000953 |
| SHANK3 | −0.000954 |
| HAVCR1 | −0.000956 |
| GSR | −0.000959 |
| SDF2L1 | −0.000959 |
| CHL1 | −0.000959 |
| TBC1D9B | −0.000960 |
| CCDC152 | −0.000961 |
| MAF1 | −0.000962 |
| CHMP1B | −0.000962 |
| SYNE4 | −0.000964 |
| DUSP13 | −0.000966 |
| IGBP1 | −0.000966 |
| MTMR10 | −0.000966 |
| CDKN1C | −0.000967 |
| SKIL | −0.000969 |
| TPPP2 | −0.000969 |
| SHISA6 | −0.000970 |
| FAM127A | −0.000970 |
| DPAGT1 | −0.000971 |
| PLP2 | −0.000971 |
| MLX | −0.000973 |
| STARD7 | −0.000973 |
| BID | −0.000973 |
| CEP112 | −0.000973 |
| MBOAT7 | −0.000974 |
| AP1G1 | −0.000975 |
| C1GALT1C1 | −0.000978 |
| NAPA | −0.000980 |
| STAT1 | −0.000980 |
| STK16 | −0.000981 |
| FKBP14 | −0.000982 |
| C7orf50 | −0.000982 |
| RUNX2 | −0.000982 |
| ZCWPW1 | −0.000983 |
| ELMSAN1 | −0.000983 |
| RPN2 | −0.000986 |
| OR1L8 | −0.000986 |
| NKIRAS1 | −0.000987 |
| PEX11A | −0.000988 |
| AP3S2 | −0.000988 |
| ZBTB22 | −0.000989 |
| TRIM4 | −0.000991 |
| CIDEC | −0.000991 |
| PGA3 | −0.000992 |
| CTAGE5 | −0.000992 |
| RGS7BP | −0.000993 |
| MAPK1 | −0.000994 |
| ELP3 | −0.000998 |
| LRRC3C | −0.001000 |
| LRRFIP2 | −0.001001 |
| UBE2E1 | −0.001003 |
| ZNF710 | −0.001003 |
| AP2A2 | −0.001004 |
| PKD1L1 | −0.001004 |
| TNFRSF14 | −0.001005 |
| TSTD1 | −0.001005 |
| RNF139 | −0.001006 |
| RPL7A | −0.001006 |
| CID | −0.001006 |
| ACSL3 | −0.001008 |
| FGB | −0.001010 |
| CLP1 | −0.001010 |
| STAP2 | −0.001010 |
| MBD2 | −0.001013 |
| TRPS1 | −0.001015 |
| ARCN1 | −0.001017 |
| PEBP1 | −0.001021 |
| SH3RF3 | −0.001022 |
| RIOK2 | −0.001022 |
| NHEJ1 | −0.001023 |
| PARP10 | −0.001024 |
| RPS6 | −0.001025 |
| SYF2 | −0.001025 |
| PADI1 | −0.001027 |
| PMM2 | −0.001029 |
| PAX7 | −0.001030 |
| MYBPC3 | −0.001031 |
| GLCE | −0.001033 |
| TMEM42 | −0.001033 |
| TTC9 | −0.001034 |
| DGCR6L | −0.001037 |
| SSX5 | −0.001037 |
| CLEC19A | −0.001040 |
| C6orf1 | −0.001040 |
| DNTTIP1 | −0.001041 |
| TMEM128 | −0.001042 |
| FPGS | −0.001043 |
| CSDE1 | −0.001044 |
| IK | −0.001044 |
| SLFNL1 | −0.001044 |
| TRAK1 | −0.001045 |
| HSH2D | −0.001045 |
| KLHL20 | −0.001047 |
| NME6 | −0.001048 |
| C5orf15 | −0.001048 |
| C7orf77 | −0.001049 |
| UQCRC1 | −0.001049 |
| GLIS1 | −0.001050 |
| PITPNM1 | −0.001051 |
| TMED1 | −0.001051 |
| PCYT1A | −0.001051 |
| UBB | −0.001051 |
| VGLL4 | −0.001052 |
| TMEM129 | −0.001053 |
| PSEN1 | −0.001055 |
| SMPDL3A | −0.001056 |
| LYAR | −0.001056 |
| RNF10 | −0.001057 |
| BLOC1S4 | −0.001058 |
| HRH4 | −0.001060 |
| CD72 | −0.001060 |
| ARL8A | −0.001063 |
| ECHS1 | −0.001064 |
| NDP | −0.001065 |
| TBL2 | −0.001066 |
| SARS | −0.001066 |
| KCNN2 | −0.001066 |
| RPS20 | −0.001069 |
| CLPTM1L | −0.001069 |
| CAPZA1 | −0.001070 |
| GPR33 | −0.001071 |
| SSR2 | −0.001071 |
| RASAL2 | −0.001071 |
| ADCK2 | −0.001071 |
| FATE1 | −0.001072 |
| IFNGR2 | −0.001073 |
| CPEB4 | −0.001073 |
| FNDC3A | −0.001073 |
| WNT2B | −0.001073 |
| TMEM50B | −0.001074 |
| TRAF3IP1 | −0.001075 |
| CA5B | −0.001076 |
| TDRD7 | −0.001076 |
| ENPP4 | −0.001077 |
| PEX11B | −0.001079 |
| GMPR | −0.001079 |
| CLEC11A | −0.001079 |
| RAB3GAP1 | −0.001080 |
| SEC23IP | −0.001080 |
| OGFR | −0.001081 |
| SAP18 | −0.001082 |
| SHARPIN | −0.001083 |
| CADPS2 | −0.001083 |
| MFSD2B | −0.001084 |
| CC2D2B | −0.001086 |
| WNT4 | −0.001087 |
| SEL1L | −0.001089 |
| ATP1B3 | −0.001090 |
| CPNE3 | −0.001090 |
| POLR2E | −0.001090 |
| RPL13A | −0.001091 |
| KDM6B | −0.001092 |
| SPOP | −0.001092 |
| ZBTB7B | −0.001092 |
| NOS2 | −0.001093 |
| IFNL1 | −0.001095 |
| ZNF524 | −0.001096 |
| PRKD2 | −0.001096 |
| BLK | −0.001096 |
| ABRA | −0.001097 |
| GCSAM | −0.001097 |
| WNT3 | −0.001098 |
| HOXA3 | −0.001098 |
| EMC4 | −0.001098 |
| FAM50B | −0.001099 |
| EIF3I | −0.001099 |
| SEPHS2 | −0.001100 |
| SRP14 | −0.001103 |
| RPL39 | −0.001103 |
| KZF4 | −0.001103 |
| KCNT2 | −0.001104 |
| HOMEZ | −0.001104 |
| CHST2 | −0.001106 |
| SNUPN | −0.001106 |
| PTPN9 | −0.001107 |
| PIGT | −0.001107 |
| IL26 | −0.001107 |
| DALRD3 | −0.001107 |
| PCK2 | −0.001108 |
| ZMYND11 | −0.001108 |
| PUS10 | −0.001108 |
| SUCLG1 | −0.001110 |
| 43710 | −0.001110 |
| BNIP3L | −0.001111 |
| GPM6B | −0.001111 |
| APOBEC2 | −0.001111 |
| ACTG1 | −0.001112 |
| SPTAN1 | −0.001112 |
| BECN1 | −0.001115 |
| TUFM | −0.001116 |
| UBE2V1 | −0.001118 |
| ATP8A1 | −0.001120 |
| BTF3 | −0.001121 |
| ENO1 | −0.001121 |
| CCNI2 | −0.001123 |
| NOMO1 | −0.001124 |
| EPS15 | −0.001124 |
| RPS3A | −0.001125 |
| UNC50 | −0.001126 |
| EMC2 | −0.001127 |
| CDC26 | −0.001127 |
| ATP5G2 | −0.001127 |
| FBX039 | −0.001129 |
| AHSP | −0.001129 |
| GALNT11 | −0.001131 |
| XIRP2 | −0.001132 |
| C2CD2 | −0.001132 |
| ARHGEF38 | −0.001135 |
| MAP2K5 | −0.001136 |
| SCARB2 | −0.001136 |
| RRAGC | −0.001137 |
| UBE2D4 | −0.001138 |
| UFM1 | −0.001138 |
| ZER1 | −0.001139 |
| CRTC3 | −0.001139 |
| OXER1 | −0.001140 |
| MAN1C1 | −0.001141 |
| DCAF6 | −0.001142 |
| PIP4K2C | −0.001144 |
| C10orf99 | −0.001144 |
| ACBD3 | −0.001145 |
| ATG101 | −0.001145 |
| ENPP5 | −0.001145 |
| TMEM189 | −0.001145 |
| DAD1 | −0.001145 |
| MKRN2OS | −0.001146 |
| SMPX | −0.001147 |
| TMEM91 | −0.001148 |
| TVP23B | −0.001148 |
| KDELR1 | −0.001151 |
| RIT1 | −0.001154 |
| ANKUB1 | −0.001154 |
| RFX5 | −0.001154 |
| TNFSF18 | −0.001156 |
| EIF4H | −0.001160 |
| RGS3 | −0.001161 |
| CREB5 | −0.001164 |
| TMEM192 | −0.001164 |
| PAM | −0.001165 |
| APLN | −0.001167 |
| MPC1 | −0.001167 |
| SLCO4A1 | −0.001167 |
| DENND5A | −0.001168 |
| AGFG2 | −0.001171 |
| MPPE1 | −0.001171 |
| BEST1 | −0.001171 |
| TMF1 | −0.001172 |
| NUB1 | −0.001172 |
| HNRNPH2 | −0.001173 |
| POMP | −0.001173 |
| SETD3 | −0.001173 |
| ANAPC13 | −0.001177 |
| SPATC1 | −0.001177 |
| MYO1E | −0.001180 |
| ZNF727 | −0.001183 |
| ZNF705A | −0.001183 |
| ATF7 | −0.001186 |
| TAPT1 | −0.001186 |
| SLC3A2 | −0.001187 |
| EID1 | −0.001190 |
| CCDC85B | −0.001190 |
| TTC25 | −0.001191 |
| ESRRA | −0.001192 |
| HDDC3 | −0.001193 |
| PTRH1 | −0.001193 |
| RPS17 | −0.001193 |
| RPSA | −0.001193 |
| SGCE | −0.001194 |
| INSL3 | −0.001194 |
| ARHGAP17 | −0.001195 |
| PNPO | −0.001197 |
| ARNT | −0.001199 |
| PRDM5 | −0.001200 |
| FABP5 | −0.001202 |
| CCT6B | −0.001204 |
| RPS25 | −0.001205 |
| GH1 | −0.001207 |
| BTG1 | −0.001207 |
| FCGR2C | −0.001209 |
| CHCHD7 | −0.001210 |
| NANS | −0.001210 |
| SEC24A | −0.001211 |
| MPV17L | −0.001211 |
| ADHFE1 | −0.001211 |
| FAM120A | −0.001212 |
| NAA38 | −0.001213 |
| KLF3 | −0.001214 |
| TACR1 | −0.001215 |
| GUK1 | −0.001217 |
| CRIP2 | −0.001219 |
| TRIP4 | −0.001219 |
| SHOC2 | −0.001219 |
| SUPT4H1 | −0.001219 |
| SLC6A2 | −0.001222 |
| LRRC59 | −0.001222 |
| RPS11 | −0.001223 |
| RAB42 | −0.001223 |
| KARS | −0.001225 |
| PLCB3 | −0.001225 |
| CCL26 | −0.001227 |
| FBXO4 | −0.001227 |
| PSORS1C2 | −0.001228 |
| MYL2 | −0.001229 |
| RAB21 | −0.001230 |
| EPN2 | −0.001230 |
| KIF6 | −0.001230 |
| TOMM20L | −0.001230 |
| DDOST | −0.001232 |
| MEPIA | −0.001233 |
| PEX19 | −0.001234 |
| NBPF19 | −0.001236 |
| IL4 | −0.001236 |
| TFIP11 | −0.001237 |
| C3orf33 | −0.001238 |
| EIF4EBP2 | −0.001238 |
| ERGIC3 | −0.001240 |
| MPV17 | −0.001240 |
| DOCK10 | −0.001240 |
| C3orf18 | −0.001243 |
| C11orf1 | −0.001243 |
| GRB14 | −0.001244 |
| CDX1 | −0.001244 |
| EPB42 | −0.001245 |
| TEX261 | −0.001246 |
| WBSCR27 | −0.001246 |
| NRBP1 | −0.001247 |
| FHDC1 | −0.001247 |
| PEX5 | −0.001248 |
| PRICKLE3 | −0.001251 |
| VPREB3 | −0.001253 |
| MMP1 | −0.001254 |
| ACTR3C | −0.001255 |
| FEM1C | −0.001256 |
| PPRC1 | −0.001258 |
| OLFML2B | −0.001258 |
| RHAD1 | −0.001259 |
| GSPT1 | −0.001259 |
| PDE7B | −0.001259 |
| OBSCN | −0.001260 |
| GYG1 | −0.001261 |
| PPIAL4C | −0.001261 |
| GNPDA1 | −0.001261 |
| GPR42 | −0.001261 |
| PLA2G7 | −0.001262 |
| GNPTAB | −0.001262 |
| NOTO | −0.001263 |
| TSR3 | −0.001264 |
| EXOC6B | −0.001265 |
| PCDH9 | −0.001265 |
| MCEE | −0.001265 |
| TNFAIP2 | −0.001266 |
| UQCR10 | −0.001266 |
| RPS4X | −0.001266 |
| R3HDML | −0.001267 |
| COL1A2 | −0.001268 |
| RPS18 | −0.001269 |
| DIRC2 | −0.001270 |
| CTRB1 | −0.001270 |
| SLC39A13 | −0.001270 |
| G6PC3 | −0.001272 |
| ZNF503 | −0.001272 |
| ADORA2A | −0.001274 |
| H3F3C | −0.001274 |
| PEX12 | −0.001274 |
| NDRG1 | −0.001274 |
| CCDC3 | −0.001274 |
| DDX58 | −0.001276 |
| TBC1D3E | −0.001280 |
| GCC1 | −0.001281 |
| GRPEL1 | −0.001281 |
| SH3YL1 | −0.001281 |
| DERA | −0.001283 |
| KDELR2 | −0.001283 |
| FAM3C | −0.001284 |
| PER2 | −0.001284 |
| CPT1A | −0.001284 |
| TVP23A | −0.001285 |
| LUZP6 | −0.001285 |
| PEX13 | −0.001286 |
| DTX3L | −0.001288 |
| PLEKHM2 | −0.001290 |
| ASCC2 | −0.001290 |
| COMMD8 | −0.001291 |
| BAK1 | −0.001292 |
| MAGEE1 | −0.001293 |
| RNF208 | −0.001294 |
| C5orf67 | −0.001295 |
| CYP2A7 | −0.001296 |
| FHL2 | −0.001297 |
| ACBD4 | −0.001298 |
| ZNF792 | −0.001299 |
| AKAP3 | −0.001299 |
| NDUFAF2 | −0.001301 |
| APOA5 | −0.001303 |
| OGFOD1 | −0.001305 |
| SIRT3 | −0.001306 |
| EEF2KMT | −0.001308 |
| UGCG | −0.001308 |
| VPS39 | −0.001308 |
| DPP9 | −0.001311 |
| STX5 | −0.001313 |
| RPS16 | −0.001313 |
| FICD | −0.001313 |
| DNAJA2 | −0.001314 |
| NFATC3 | −0.001315 |
| CT45A10 | −0.001316 |
| WFDC11 | −0.001316 |
| FDPS | −0.001316 |
| TM9SF1 | −0.001316 |
| ZNF676 | −0.001317 |
| RPS24 | −0.001317 |
| GAL3ST4 | −0.001318 |
| APOBEC3F | −0.001318 |
| AGAP2 | −0.001320 |
| MAP2K1 | −0.001321 |
| C17orf49 | −0.001321 |
| FHIT | −0.001322 |
| SERP1 | −0.001323 |
| DNAJB9 | −0.001323 |
| CTTNBP2NL | −0.001324 |
| SSSCA1 | −0.001324 |
| STX19 | −0.001325 |
| SMG6 | −0.001327 |
| FBXW5 | −0.001327 |
| FAM180B | −0.001328 |
| KBTBD8 | −0.001330 |
| AKR1B15 | −0.001332 |
| CHMP4A | −0.001333 |
| FAM96B | −0.001333 |
| RWDD2B | −0.001333 |
| KCTD18 | −0.001334 |
| HEATR5A | −0.001334 |
| INSIG2 | −0.001334 |
| CD163L1 | −0.001335 |
| RAP2C | −0.001337 |
| OSTN | −0.001338 |
| ACER3 | −0.001339 |
| MOBIB | −0.001339 |
| INO80 | −0.001340 |
| IL22RA2 | −0.001341 |
| THRA | −0.001341 |
| TGM4 | −0.001341 |
| RPLP2 | −0.001342 |
| CPT2 | −0.001343 |
| BSG | −0.001343 |
| LPIN2 | −0.001344 |
| PIR | −0.001346 |
| FAM118B | −0.001350 |
| UROD | −0.001350 |
| UBE2L3 | −0.001351 |
| LMO2 | −0.001352 |
| SPCS3 | −0.001352 |
| CSK | −0.001352 |
| PIGBOS1 | −0.001352 |
| ACACB | −0.001354 |
| SYNDIG1 | −0.001355 |
| HECW1 | −0.001355 |
| C16orf62 | −0.001355 |
| TRAPPC2L | −0.001355 |
| PPP4R1 | −0.001356 |
| CHMP5 | −0.001356 |
| CNKSR1 | −0.001359 |
| TCAIM | −0.001359 |
| KIAA1324L | −0.001359 |
| ODF3L2 | −0.001361 |
| AGFG1 | −0.001361 |
| CCNY | −0.001362 |
| SLC11A2 | −0.001363 |
| PGAM1 | −0.001364 |
| SLN | −0.001364 |
| HEXIM1 | −0.001365 |
| ALDH6A1 | −0.001365 |
| NBEAL1 | −0.001366 |
| SLC39A12 | −0.001366 |
| DNMBP | −0.001366 |
| CERCAM | −0.001366 |
| ATF1 | −0.001367 |
| MBTPS1 | −0.001367 |
| TCF25 | −0.001368 |
| RP2 | −0.001368 |
| CHPF2 | −0.001371 |
| KIF13B | −0.001374 |
| ARL1 | −0.001374 |
| GBF1 | −0.001375 |
| ITGAE | −0.001376 |
| DNASE1L1 | −0.001376 |
| ESYT2 | −0.001378 |
| ZNF860 | −0.001380 |
| ALG12 | −0.001380 |
| RFT1 | −0.001381 |
| NDUFB8 | −0.001382 |
| VAC14 | −0.001382 |
| GFRA3 | −0.001382 |
| SIK2 | −0.001386 |
| CTNNAL1 | −0.001387 |
| TRIM40 | −0.001389 |
| MONIA | −0.001389 |
| HM13 | −0.001390 |
| EEF1G | −0.001391 |
| GJB1 | −0.001394 |
| BCKDK | −0.001396 |
| IGSF8 | −0.001398 |
| CDNF | −0.001398 |
| SERP2 | −0.001399 |
| FLOT1 | −0.001400 |
| LRRC70 | −0.001401 |
| PRICKLE4 | −0.001401 |
| SCN1B | −0.001402 |
| PPM1J | −0.001403 |
| CDK18 | −0.001404 |
| CRADD | −0.001405 |
| OXR1 | −0.001406 |
| SEMA6B | −0.001407 |
| SMDT1 | −0.001407 |
| SLC35F2 | −0.001407 |
| DNAH2 | −0.001409 |
| BTG3 | −0.001409 |
| C17orf50 | −0.001411 |
| ZSWIM7 | −0.001411 |
| RGS16 | −0.001413 |
| DACT1 | −0.001413 |
| AIG1 | −0.001414 |
| LRIG3 | −0.001416 |
| CHMP1A | −0.001416 |
| GIT2 | −0.001416 |
| STAM | −0.001418 |
| TNNI1 | −0.001418 |
| CITED4 | −0.001418 |
| ERLIN1 | −0.001421 |
| TGFB3 | −0.001422 |
| CEMIP | −0.001423 |
| BAG2 | −0.001427 |
| BMP15 | −0.001427 |
| GLA | −0.001427 |
| MRPS36 | −0.001428 |
| CRYBB1 | −0.001428 |
| PLEKHA1 | −0.001428 |
| C11orf54 | −0.001431 |
| SLC25A11 | −0.001432 |
| CDV3 | −0.001435 |
| SLC10A6 | −0.001436 |
| NTAN1 | −0.001439 |
| AP3B1 | −0.001440 |
| C17orf105 | −0.001441 |
| WASF2 | −0.001442 |
| PTPRJ | −0.001444 |
| TMEM8A | −0.001444 |
| PLOD3 | −0.001447 |
| STAM2 | −0.001447 |
| RPL26 | −0.001447 |
| HAPLN3 | −0.001448 |
| FAM117A | −0.001448 |
| FHAD1 | −0.001449 |
| OAZ1 | −0.001449 |
| OPLAH | −0.001454 |
| PICALM | −0.001454 |
| TXNRD2 | −0.001454 |
| SLC35A2 | −0.001454 |
| NSMCE1 | −0.001456 |
| NRN1 | −0.001458 |
| ALDH7A1 | −0.001460 |
| FAM45A | −0.001461 |
| TXNDC5 | −0.001461 |
| APP | −0.001462 |
| PYROXD2 | −0.001463 |
| HACL1 | −0.001463 |
| NOX1 | −0.001464 |
| SLC9A8 | −0.001464 |
| CCDC97 | −0.001466 |
| SF3A1 | −0.001467 |
| LRRC19 | −0.001468 |
| ORMDL3 | −0.001468 |
| ACKR3 | −0.001469 |
| ATP6AP2 | −0.001471 |
| MINK1 | −0.001472 |
| FBXO7 | −0.001473 |
| RHCG | −0.001473 |
| OR52N2 | −0.001474 |
| FDXACB1 | −0.001474 |
| TMEM86A | −0.001474 |
| GPCPD1 | −0.001475 |
| HSPA5 | −0.001475 |
| FKBP1A | −0.001476 |
| MEIS2 | −0.001477 |
| OGFRL1 | −0.001479 |
| OVOL1 | −0.001480 |
| CHCHD4 | −0.001483 |
| TCP11 | −0.001484 |
| ARMC9 | −0.001486 |
| DEPTOR | −0.001486 |
| TLN2 | −0.001487 |
| ACTR1A | −0.001488 |
| LBX1 | −0.001489 |
| MACF1 | −0.001490 |
| FAM63B | −0.001493 |
| LYRM1 | −0.001493 |
| ISOC2 | −0.001494 |
| SLC03A1 | −0.001494 |
| KAZN | −0.001498 |
| SLC30A1 | −0.001499 |
| NIPAL2 | −0.001503 |
| MAGT1 | −0.001504 |
| SLCO1B3 | −0.001506 |
| ANO5 | −0.001508 |
| CAMTA2 | −0.001508 |
| PHF23 | −0.001508 |
| TMEM30B | −0.001509 |
| POLR2A | −0.001509 |
| SAMD14 | −0.001512 |
| SNTB2 | −0.001513 |
| ANKRD40 | −0.001513 |
| ASB11 | −0.001513 |
| NCBP2L | −0.001515 |
| PRR9 | −0.001515 |
| CYB5R1 | −0.001516 |
| ROB03 | −0.001517 |
| DTWD2 | −0.001518 |
| CDIPT | −0.001518 |
| ADAMTS4 | −0.001520 |
| GNB4 | −0.001522 |
| MRPL24 | −0.001523 |
| YWHAB | −0.001523 |
| CPS1 | −0.001524 |
| VTN | −0.001525 |
| RRAGA | −0.001525 |
| FARS2 | −0.001526 |
| TANGO6 | −0.001528 |
| PDLIM7 | −0.001528 |
| ERP44 | −0.001528 |
| THSD7A | −0.001531 |
| FKBP2 | −0.001532 |
| OR8S1 | −0.001534 |
| ATN1 | −0.001534 |
| PRDM11 | −0.001536 |
| THOC7 | −0.001536 |
| PHACTR3 | −0.001536 |
| ZFYVE21 | −0.001536 |
| ZDHHC12 | −0.001537 |
| SUSD6 | −0.001539 |
| THAP11 | −0.001540 |
| TRAPPC6A | −0.001541 |
| SETD9 | −0.001541 |
| CMPK1 | −0.001542 |
| MUCH | −0.001542 |
| ANO2 | −0.001542 |
| NAT8B | −0.001543 |
| NXPE3 | −0.001547 |
| MGRN1 | −0.001548 |
| ST13 | −0.001549 |
| GDPGP1 | −0.001551 |
| SUFU | −0.001552 |
| FAM114A2 | −0.001553 |
| FZD6 | −0.001553 |
| SCT | −0.001553 |
| GNA12 | −0.001554 |
| S1PR5 | −0.001555 |
| ENO4 | −0.001555 |
| ARHGEF18 | −0.001558 |
| DAO | −0.001558 |
| FBXL7 | −0.001559 |
| PTCHD4 | −0.001559 |
| MAF | −0.001560 |
| GNG10 | −0.001560 |
| NMRK2 | −0.001560 |
| MT-CYB | −0.001561 |
| ZFYVE28 | −0.001562 |
| TXNDC17 | −0.001563 |
| DDIT4 | −0.001563 |
| PSENEN | −0.001563 |
| TM7SF3 | −0.001564 |
| ATL3 | −0.001564 |
| SNAPC5 | −0.001567 |
| SLX1B | −0.001567 |
| DGKK | −0.001570 |
| C1orf115 | −0.001572 |
| ATP6AP1 | −0.001572 |
| RAP1B | −0.001572 |
| CYP2C9 | −0.001573 |
| MRPL43 | −0.001573 |
| CYB561 | −0.001575 |
| SSH1 | −0.001575 |
| GATM | −0.001575 |
| CCDC181 | −0.001576 |
| HOGA1 | −0.001577 |
| IL17RC | −0.001577 |
| RIPK4 | −0.001578 |
| KBTBD11 | −0.001579 |
| MFN2 | −0.001579 |
| CALU | −0.001580 |
| INSC | −0.001580 |
| CEACAM5 | −0.001581 |
| RANGRF | −0.001581 |
| APH1A | −0.001584 |
| C19orf70 | −0.001584 |
| MEIS1 | −0.001584 |
| DHRS1 | −0.001584 |
| HAL | −0.001585 |
| ADAMTS17 | −0.001586 |
| VPS13D | −0.001586 |
| SAMD11 | −0.001586 |
| DEXI | −0.001587 |
| TMEM41A | −0.001587 |
| PLEKHF2 | −0.001588 |
| SPCS1 | −0.001591 |
| MRPS18B | −0.001591 |
| ZNF575 | −0.001591 |
| PARP12 | −0.001592 |
| OXA1L | −0.001593 |
| ABCD3 | −0.001593 |
| PAQR8 | −0.001595 |
| TAX1BP1 | −0.001596 |
| NFU1 | −0.001598 |
| TM9SF2 | −0.001598 |
| GPR31 | −0.001598 |
| NAT6 | −0.001599 |
| PQLC1 | −0.001599 |
| RTCB | −0.001601 |
| EIF3K | −0.001601 |
| MRPL54 | −0.001601 |
| TXNDC2 | −0.001603 |
| TNN | −0.001604 |
| YIPF3 | −0.001606 |
| MYO16 | −0.001606 |
| RPL10A | −0.001607 |
| KRT39 | −0.001609 |
| ALG5 | −0.001609 |
| ANKRD42 | −0.001609 |
| EIF3D | −0.001610 |
| ZBTB7C | −0.001610 |
| MAPK13 | −0.001611 |
| MOCS2 | −0.001612 |
| MOSPD3 | −0.001613 |
| SPATA2L | −0.001614 |
| MIDN | −0.001614 |
| BAD | −0.001615 |
| FOXO4 | −0.001615 |
| UBXN1 | −0.001616 |
| SEC31A | −0.001616 |
| CCDC173 | −0.001619 |
| LRIG1 | −0.001620 |
| ZSCAN4 | −0.001622 |
| RAPGEF2 | −0.001623 |
| GPR25 | −0.001623 |
| NID1 | −0.001625 |
| FAM187B | −0.001625 |
| GCLM | −0.001626 |
| MEF2A | −0.001628 |
| PGRMC1 | −0.001629 |
| MFSD10 | −0.001630 |
| PRR32 | −0.001633 |
| HES1 | −0.001634 |
| FCHSD2 | −0.001634 |
| SCYL1 | −0.001635 |
| KCNE5 | −0.001636 |
| HDLBP | −0.001636 |
| CBY1 | −0.001636 |
| DDX28 | −0.001636 |
| SNX12 | −0.001638 |
| COL27A1 | −0.001638 |
| TP53I3 | −0.001639 |
| CAMK2G | −0.001642 |
| SEC11A | −0.001644 |
| RPL4 | −0.001647 |
| NEDD4 | −0.001647 |
| STYXL1 | −0.001647 |
| CREBRF | −0.001649 |
| SFRP5 | −0.001649 |
| CRELD1 | −0.001651 |
| SEC13 | −0.001652 |
| ATF6 | −0.001655 |
| AK7 | −0.001656 |
| TSPYL1 | −0.001657 |
| FAM83H | −0.001658 |
| TINAG | −0.001658 |
| SMCO4 | −0.001660 |
| RHBDD1 | −0.001661 |
| AIP | −0.001663 |
| TOMM22 | −0.001663 |
| RDX | −0.001663 |
| ZDHHC5 | −0.001664 |
| OAT | −0.001664 |
| MVD | −0.001669 |
| RPS27L | −0.001670 |
| FITM2 | −0.001672 |
| ATPIA4 | −0.001673 |
| IFNG | −0.001680 |
| PORCN | −0.001680 |
| SPINK4 | −0.001681 |
| AZI2 | −0.001682 |
| ERMAP | −0.001682 |
| DRAM2 | −0.001683 |
| SESN1 | −0.001684 |
| ATAD3C | −0.001685 |
| YIPF1 | −0.001687 |
| DCHS2 | −0.001687 |
| ALDH9A1 | −0.001688 |
| PPIAL4A | −0.001688 |
| CLN8 | −0.001689 |
| NXT2 | −0.001689 |
| VIP | −0.001691 |
| TSSK2 | −0.001693 |
| LYPLA2 | −0.001694 |
| TAF4B | −0.001695 |
| MYOM1 | −0.001696 |
| RSPH10B | −0.001697 |
| ADIPOR1 | −0.001699 |
| AQP8 | −0.001699 |
| VAPA | −0.001701 |
| ALDH4A1 | −0.001702 |
| GYS1 | −0.001703 |
| UROS | −0.001704 |
| UBL7 | −0.001705 |
| PARP9 | −0.001705 |
| IGF1R | −0.001708 |
| CYP4A11 | −0.001708 |
| FRMD6 | −0.001710 |
| FUT11 | −0.001710 |
| VPS26A | −0.001710 |
| LZTFL1 | −0.001710 |
| SPACA4 | −0.001711 |
| LRRC3 | −0.001711 |
| USP50 | −0.001711 |
| PTPMT1 | −0.001712 |
| ESYT1 | −0.001713 |
| DFNA5 | −0.001715 |
| MOCS1 | −0.001718 |
| CLINT1 | −0.001718 |
| ACR | −0.001720 |
| ENTPD1 | −0.001723 |
| MAP3K7CL | −0.001723 |
| C4orf36 | −0.001728 |
| FOXJ2 | −0.001729 |
| MYDGF | −0.001730 |
| NDFIP2 | −0.001730 |
| 43714 | −0.001732 |
| TACR2 | −0.001733 |
| CFAP57 | −0.001735 |
| FKBP8 | −0.001737 |
| MPO | −0.001738 |
| JAZF1 | −0.001738 |
| CCNB3 | −0.001739 |
| IGSF3 | −0.001740 |
| SCAMP3 | −0.001740 |
| SERTAD2 | −0.001741 |
| GRIA3 | −0.001741 |
| CCDC7 | −0.001742 |
| RPS27 | −0.001743 |
| ARHGEF28 | −0.001743 |
| EEF1D | −0.001744 |
| PARN | −0.001746 |
| GMPR2 | −0.001746 |
| CAP2 | −0.001748 |
| RSPH14 | −0.001749 |
| GYPB | −0.001752 |
| NLRP7 | −0.001755 |
| GFOD1 | −0.001756 |
| FZD10 | −0.001758 |
| LAMTOR2 | −0.001758 |
| WNK1 | −0.001761 |
| CHST12 | −0.001761 |
| CUTC | −0.001763 |
| MGST2 | −0.001763 |
| RPS3 | −0.001763 |
| P4HB | −0.001764 |
| DNAJC30 | −0.001764 |
| CHAD | −0.001765 |
| EFHD2 | −0.001765 |
| IL36RN | −0.001767 |
| CYP51A1 | −0.001767 |
| DIRAS3 | −0.001772 |
| PRKAB1 | −0.001773 |
| LYPLAL1 | −0.001774 |
| NBR1 | −0.001774 |
| RPL11 | −0.001774 |
| ARF1 | −0.001775 |
| PPCS | −0.001776 |
| BHMT2 | −0.001776 |
| CYP2U1 | −0.001777 |
| HBZ | −0.001779 |
| SCO2 | −0.001781 |
| ATXN7 | −0.001782 |
| STARD3NL | −0.001783 |
| ANXA9 | −0.001784 |
| DCTD | −0.001784 |
| EML2 | −0.001786 |
| CHTF8 | −0.001789 |
| RHOBTB1 | −0.001790 |
| PLVAP | −0.001790 |
| UNC5B | −0.001791 |
| AAR2 | −0.001793 |
| HIGD2A | −0.001797 |
| DIXDC1 | −0.001798 |
| CTTNBP2 | −0.001798 |
| GDF3 | −0.001799 |
| PLXNC1 | −0.001800 |
| CLIP1 | −0.001801 |
| SLC41A1 | −0.001802 |
| MAP3K3 | −0.001802 |
| CHPF | −0.001805 |
| CREM | −0.001806 |
| AIM1L | −0.001807 |
| CALCOCO1 | −0.001811 |
| HSD17B13 | −0.001811 |
| GSPT2 | −0.001812 |
| FBXO8 | −0.001813 |
| TADA2B | −0.001816 |
| ATOX1 | −0.001816 |
| PTPN12 | −0.001817 |
| SPINK6 | −0.001819 |
| EPB41L3 | −0.001819 |
| MMP8 | −0.001820 |
| SLC24A1 | −0.001822 |
| BBOX1 | −0.001823 |
| LLGL2 | −0.001825 |
| TPCN1 | −0.001827 |
| SPRR3 | −0.001828 |
| PTCHD3 | −0.001828 |
| CETN2 | −0.001828 |
| NEO1 | −0.001828 |
| RPL9 | −0.001829 |
| EIF5B | −0.001830 |
| OLFM2 | −0.001830 |
| WIPF2 | −0.001830 |
| FZD2 | −0.001831 |
| HBP1 | −0.001832 |
| RREB1 | −0.001832 |
| MX2 | −0.001833 |
| EML3 | −0.001837 |
| CRISP2 | −0.001837 |
| BSCL2 | −0.001839 |
| BEX5 | −0.001842 |
| PNMA2 | −0.001843 |
| MIPEP | −0.001844 |
| FAM174B | −0.001844 |
| CYLD | −0.001844 |
| NELL2 | −0.001845 |
| TXK | −0.001847 |
| SMAP2 | −0.001847 |
| ANKRD13A | −0.001851 |
| PPP1R3G | −0.001851 |
| CDC42SE2 | −0.001853 |
| ACOX1 | −0.001853 |
| MMP9 | −0.001853 |
| MTMR6 | −0.001854 |
| RPL34 | −0.001855 |
| TRIM14 | −0.001855 |
| RASA3 | −0.001857 |
| RAB5A | −0.001859 |
| CYB5B | −0.001861 |
| LIX1L | −0.001863 |
| CDC14B | −0.001864 |
| ZNF217 | −0.001864 |
| FAM124A | −0.001864 |
| GPR15 | −0.001864 |
| ACO2 | −0.001865 |
| WDR66 | −0.001867 |
| TTC38 | −0.001867 |
| PXK | −0.001868 |
| AHCYL1 | −0.001869 |
| MYLPF | −0.001869 |
| DAP | −0.001870 |
| PIP4K2A | −0.001872 |
| IL37 | −0.001874 |
| GINM1 | −0.001874 |
| MORF4L1 | −0.001878 |
| PPIAL4D | −0.001882 |
| CXorf57 | −0.001883 |
| NUDT9 | −0.001884 |
| UEVLD | −0.001884 |
| ACTR3 | −0.001885 |
| CAMK2D | −0.001888 |
| GORASP2 | −0.001893 |
| SETD7 | −0.001894 |
| PIM2 | −0.001894 |
| NIP7 | −0.001894 |
| SOWAHB | −0.001894 |
| PALM | −0.001895 |
| FNDC4 | −0.001896 |
| NSDHL | −0.001896 |
| FKBP10 | −0.001896 |
| DBNDD2 | −0.001897 |
| GBP7 | −0.001898 |
| CHCHD5 | −0.001899 |
| EXTL1 | −0.001902 |
| RAPGEF5 | −0.001903 |
| SLC38A10 | −0.001903 |
| SLC12A2 | −0.001903 |
| CTH | −0.001903 |
| UGP2 | −0.001904 |
| RPL31 | −0.001907 |
| IGIP | −0.001908 |
| SLC38A4 | −0.001909 |
| EIF1 | −0.001911 |
| CLIC4 | −0.001911 |
| SHKBP1 | −0.001913 |
| ZFYVE9 | −0.001914 |
| SLC45A4 | −0.001918 |
| LIFR | −0.001921 |
| MPP3 | −0.001922 |
| FXYD7 | −0.001923 |
| FFAR3 | −0.001923 |
| SEC16B | −0.001924 |
| TPS3 | −0.001925 |
| PJA2 | −0.001925 |
| NTSC1A | −0.001926 |
| SERINC4 | −0.001926 |
| KDSR | −0.001926 |
| PHLDA1 | −0.001927 |
| PTP4A2 | −0.001927 |
| TRAF3IP2 | −0.001928 |
| CCDC88B | −0.001932 |
| C10orf76 | −0.001933 |
| SERPINB10 | −0.001935 |
| MPZL1 | −0.001937 |
| LACTB2 | −0.001937 |
| USP4 | −0.001937 |
| ALDOA | −0.001938 |
| ARMCX1 | −0.001941 |
| SPG21 | −0.001942 |
| UTP3 | −0.001946 |
| CTNNBIP1 | −0.001947 |
| TMEM233 | −0.001947 |
| APOL4 | −0.001949 |
| PPARGC1A | −0.001949 |
| DDX19B | −0.001952 |
| COMMD6 | −0.001953 |
| RNF185 | −0.001953 |
| H1FNT | −0.001953 |
| COA3 | −0.001954 |
| SLC22A10 | −0.001955 |
| LINGO4 | −0.001960 |
| BLOC1S2 | −0.001961 |
| TRAK2 | −0.001962 |
| NAPRT | −0.001965 |
| TTC1 | −0.001966 |
| NOMO2 | −0.001966 |
| SLMAP | −0.001966 |
| PTEN | −0.001967 |
| FOXP3 | −0.001967 |
| RPL27 | −0.001968 |
| JUN | −0.001968 |
| DHRS4L2 | −0.001970 |
| PPP1R1C | −0.001970 |
| PECR | −0.001971 |
| LACC1 | −0.001972 |
| RPL19 | −0.001974 |
| ARHGEF12 | −0.001977 |
| EBAG9 | −0.001978 |
| DNAH3 | −0.001979 |
| CDSN | −0.001982 |
| OST4 | −0.001984 |
| PPFIBP2 | −0.001984 |
| TWSG1 | −0.001985 |
| YIF1A | −0.001985 |
| ARL5A | −0.001986 |
| MRAP | −0.001986 |
| CCDC47 | −0.001987 |
| RNF167 | −0.001988 |
| CHURC1 | −0.001988 |
| STEAP3 | −0.001988 |
| LYRM9 | −0.001989 |
| ALDH1L1 | −0.001990 |
| PELO | −0.001991 |
| SBK3 | −0.001991 |
| CREB3 | −0.001991 |
| CTAGE15 | −0.001992 |
| RASGRF2 | −0.001992 |
| IFRD2 | −0.001992 |
| FTO | −0.001993 |
| DEGS1 | −0.001993 |
| GNPTG | −0.001994 |
| MPG | −0.001994 |
| PELI1 | −0.001997 |
| TMEM248 | −0.001997 |
| GPX4 | −0.001998 |
| CPXCR1 | −0.002000 |
| FLNB | −0.002003 |
| PDE4DIP | −0.002004 |
| SEC24D | −0.002007 |
| ZDHHC24 | −0.002007 |
| ALS2CR12 | −0.002008 |
| CIR1 | −0.002008 |
| CYP26B1 | −0.002009 |
| TADA3 | −0.002009 |
| CORO2A | −0.002011 |
| LOXHD1 | −0.002013 |
| FFAR2 | −0.002013 |
| VTI1B | −0.002013 |
| PDIA5 | −0.002013 |
| THNSL2 | −0.002015 |
| PPP3CC | −0.002016 |
| RPL12 | −0.002016 |
| MMAA | −0.002018 |
| TP53I11 | −0.002021 |
| CLPTM1 | −0.002021 |
| ESD | −0.002022 |
| UBE2H | −0.002023 |
| FGFR1 | −0.002024 |
| FKBP15 | −0.002024 |
| CMPK2 | −0.002025 |
| FBXL3 | −0.002025 |
| UMAD1 | −0.002025 |
| ATP8B1 | −0.002026 |
| VEGFB | −0.002028 |
| SDC3 | −0.002030 |
| WFDC10A | −0.002031 |
| SLC50A1 | −0.002034 |
| ADIPOR2 | −0.002035 |
| PDIA3 | −0.002037 |
| ZNF843 | −0.002038 |
| GSG1L | −0.002040 |
| PPP1R18 | −0.002041 |
| COX4I1 | −0.002041 |
| PGLYRP1 | −0.002042 |
| WDR91 | −0.002043 |
| GALK1 | −0.002045 |
| HOMER3 | −0.002046 |
| ZAP70 | −0.002046 |
| TANK | −0.002048 |
| NDUFAF1 | −0.002048 |
| C9orf64 | −0.002048 |
| CNIH4 | −0.002050 |
| XKR8 | −0.002050 |
| ITGAV | −0.002050 |
| SEMA3F | −0.002052 |
| C14orf159 | −0.002055 |
| JOSD1 | −0.002055 |
| KRBOX1 | −0.002056 |
| TOR1AIP2 | −0.002056 |
| APOL2 | −0.002057 |
| RPL23 | −0.002057 |
| GSTM3 | −0.002057 |
| HS3ST3A1 | −0.002058 |
| SERF2 | −0.002059 |
| YWHAH | −0.002060 |
| SPPL2A | −0.002061 |
| INSL4 | −0.002064 |
| NDUFA4 | −0.002067 |
| SLC25A26 | −0.002067 |
| PRKAR1A | −0.002068 |
| MICU1 | −0.002069 |
| CHRNA6 | −0.002069 |
| TMEM51 | −0.002075 |
| IMP3 | −0.002077 |
| GSTM2 | −0.002077 |
| OPTN | −0.002078 |
| BCL2L13 | −0.002081 |
| FERMT1 | −0.002082 |
| WWP1 | −0.002083 |
| EIF3C | −0.002084 |
| ZNF330 | −0.002085 |
| MAP7D1 | −0.002085 |
| BNIP2 | −0.002085 |
| SEC62 | −0.002086 |
| UGDH | −0.002086 |
| TJP2 | −0.002087 |
| CCL16 | −0.002087 |
| ITFG1 | −0.002088 |
| ZFAND6 | −0.002088 |
| COL15A1 | −0.002089 |
| GABRP | −0.002093 |
| BTG4 | −0.002095 |
| MRPL40 | −0.002095 |
| STX7 | −0.002098 |
| CCDC12 | −0.002099 |
| FOXN3 | −0.002100 |
| DCTN1 | −0.002101 |
| PIGA | −0.002102 |
| MGST3 | −0.002104 |
| ABCD1 | −0.002104 |
| TLDC2 | −0.002105 |
| MT-ND1 | −0.002106 |
| MAN2A1 | −0.002106 |
| TCP11L2 | −0.002106 |
| TMEM187 | −0.002107 |
| ADPGK | −0.002110 |
| NRG1 | −0.002113 |
| DYDC2 | −0.002113 |
| RPL36AL | −0.002115 |
| AKR1E2 | −0.002115 |
| RPL8 | −0.002119 |
| DNPEP | −0.002120 |
| POLR3GL | −0.002123 |
| PLD2 | −0.002123 |
| FUT6 | −0.002124 |
| LEPROTL1 | −0.002124 |
| CNIH3 | −0.002126 |
| OSBPL10 | −0.002127 |
| TM2D2 | −0.002130 |
| EXOSC7 | −0.002130 |
| CFL1 | −0.002130 |
| LRRC8C | −0.002130 |
| PHKB | −0.002131 |
| TTLL10 | −0.002133 |
| RAC1 | −0.002133 |
| FLT1 | −0.002134 |
| TMED3 | −0.002136 |
| RTN4 | −0.002137 |
| CHMP2B | −0.002137 |
| PFDN1 | −0.002138 |
| DPP7 | −0.002139 |
| EVA1B | −0.002139 |
| GMIP | −0.002140 |
| STMND1 | −0.002141 |
| ZDHHC2 | −0.002142 |
| CA11 | −0.002142 |
| RAMP1 | −0.002143 |
| TMOD1 | −0.002146 |
| AAMDC | −0.002147 |
| RSPH9 | −0.002149 |
| RB1 | −0.002149 |
| ITGA6 | −0.002149 |
| BACE2 | −0.002150 |
| GLI2 | −0.002150 |
| PGAP3 | −0.002152 |
| RPL27A | −0.002152 |
| PFDN5 | −0.002153 |
| PRKG2 | −0.002154 |
| HERPUD2 | −0.002155 |
| CPTP | −0.002155 |
| PYGB | −0.002155 |
| ADRA2C | −0.002156 |
| STAB1 | −0.002156 |
| MBP | −0.002157 |
| FAU | −0.002157 |
| GAB3 | −0.002157 |
| C2orf50 | −0.002158 |
| HBE1 | −0.002160 |
| ARPC3 | −0.002160 |
| EEF1B2 | −0.002161 |
| FAM177A1 | −0.002163 |
| TMEM14C | −0.002164 |
| AXIN2 | −0.002164 |
| RBL2 | −0.002165 |
| HTR1B | −0.002167 |
| APOB | −0.002167 |
| XPC | −0.002168 |
| CACNB4 | −0.002169 |
| SHROOM3 | −0.002169 |
| MKNK1 | −0.002169 |
| FGD4 | −0.002169 |
| TESC | −0.002169 |
| KCNJ2 | −0.002169 |
| CLCA4 | −0.002171 |
| UCP2 | −0.002172 |
| CDR2 | −0.002172 |
| ZNF888 | −0.002174 |
| SLC22A15 | −0.002175 |
| PLXND1 | −0.002177 |
| CXXC5 | −0.002177 |
| MOB1A | −0.002178 |
| FRZB | −0.002178 |
| FOXC2 | −0.002178 |
| ITGA4 | −0.002179 |
| GRAMD3 | −0.002181 |
| HBG1 | −0.002182 |
| TMEM154 | −0.002183 |
| SLC4A1 | −0.002183 |
| CELA1 | −0.002184 |
| SGPP1 | −0.002184 |
| FCGBP | −0.002184 |
| IGF2R | −0.002188 |
| GLG1 | −0.002192 |
| CDH1 | −0.002193 |
| CASQ1 | −0.002193 |
| C1orf168 | −0.002194 |
| FBXL17 | −0.002195 |
| SACM1L | −0.002195 |
| CT45A6 | −0.002195 |
| TMEM164 | −0.002196 |
| RPL32 | −0.002197 |
| AP1B1 | −0.002198 |
| TMEM176B | −0.002200 |
| DYRK4 | −0.002201 |
| A4GNT | −0.002202 |
| ISPD | −0.002203 |
| ATXN1L | −0.002203 |
| BBS12 | −0.002204 |
| CD46 | −0.002205 |
| ADGRG5 | −0.002207 |
| UFSP2 | −0.002207 |
| LYSMD3 | −0.002208 |
| SNRPN | −0.002214 |
| OAS3 | −0.002214 |
| WT1 | −0.002214 |
| IDUA | −0.002217 |
| GORASP1 | −0.002219 |
| RABI4 | −0.002220 |
| RUSC2 | −0.002223 |
| SKAP2 | −0.002228 |
| SLC4A2 | −0.002229 |
| EMID1 | −0.002231 |
| TRIM58 | −0.002233 |
| RBM11 | −0.002234 |
| C14orf1 | −0.002235 |
| ABCC1 | −0.002235 |
| DUSP16 | −0.002237 |
| GDF2 | −0.002239 |
| EIF3L | −0.002241 |
| SLITRK2 | −0.002241 |
| STEAP1 | −0.002241 |
| PSMB6 | −0.002246 |
| SHBG | −0.002246 |
| FRAT1 | −0.002247 |
| POU5F1 | −0.002247 |
| 43712 | −0.002248 |
| HEMGN | −0.002248 |
| GNAT2 | −0.002249 |
| RPL10 | −0.002250 |
| VWA8 | −0.002251 |
| JMJD8 | −0.002252 |
| DEFA4 | −0.002255 |
| INPP1 | −0.002256 |
| RAB1A | −0.002256 |
| SPATA22 | −0.002258 |
| STUB1 | −0.002258 |
| WDR93 | −0.002259 |
| YIPF5 | −0.002259 |
| SPRR2A | −0.002261 |
| TBC1D2B | −0.002264 |
| CTDNEP1 | −0.002267 |
| PLXNB2 | −0.002267 |
| MYEOV | −0.002269 |
| BAHD1 | −0.002270 |
| SNX1 | −0.002275 |
| CGB7 | −0.002276 |
| WNT9A | −0.002277 |
| MKL1 | −0.002278 |
| AASS | −0.002279 |
| TMEM141 | −0.002280 |
| TMPPE | −0.002280 |
| FADS3 | −0.002281 |
| ACTR2 | −0.002283 |
| PVRIG | −0.002284 |
| CASC4 | −0.002284 |
| BLOC1S3 | −0.002288 |
| FNBP1 | −0.002288 |
| SAMD9 | −0.002288 |
| DDIT4L | −0.002289 |
| FREM1 | −0.002289 |
| KREMEN1 | −0.002290 |
| SSMEM1 | −0.002290 |
| ST3GAL2 | −0.002293 |
| JUP | −0.002295 |
| CREBL2 | −0.002295 |
| UFC1 | −0.002297 |
| PAQR7 | −0.002297 |
| BCL7B | −0.002300 |
| OOEP | −0.002300 |
| MPP5 | −0.002302 |
| PPPIR12B | −0.002304 |
| CDIP1 | −0.002304 |
| BCL2L1 | −0.002304 |
| ARIIGAP26 | −0.002305 |
| CISD3 | −0.002306 |
| ZMAT5 | −0.002306 |
| TOX2 | −0.002308 |
| TBX3 | −0.002309 |
| CTDSP2 | −0.002309 |
| SREK1IP1 | −0.002310 |
| EFHD1 | −0.002311 |
| IPCEF1 | −0.002312 |
| CRK | −0.002313 |
| TMEM59 | −0.002315 |
| ITGA10 | −0.002316 |
| VIPR2 | −0.002317 |
| APEH | −0.002319 |
| ARPC5 | −0.002319 |
| GUCY1A3 | −0.002320 |
| C11orf74 | −0.002320 |
| PPIB | −0.002321 |
| TMED9 | −0.002321 |
| UBLCP1 | −0.002322 |
| RPS12 | −0.002323 |
| TBKBP1 | −0.002325 |
| CDR2L | −0.002326 |
| TBC1D5 | −0.002326 |
| ASMTL | −0.002331 |
| C9orf116 | −0.002331 |
| CDC42EP3 | −0.002335 |
| C14orf119 | −0.002335 |
| RPS6KA3 | −0.002336 |
| TRIM16L | −0.002337 |
| SNAI1 | −0.002339 |
| BPI | −0.002340 |
| TMEM208 | −0.002340 |
| SREBF2 | −0.002342 |
| NFIA | −0.002343 |
| RASSF4 | −0.002343 |
| TMED7 | −0.002345 |
| STX4 | −0.002347 |
| DENND1B | −0.002348 |
| MANSC1 | −0.002349 |
| AFF3 | −0.002349 |
| AKAP12 | −0.002352 |
| EHD3 | −0.002353 |
| GP6 | −0.002354 |
| IL12A | −0.002355 |
| SERTAD4 | −0.002355 |
| SDHD | −0.002356 |
| FAM63A | −0.002356 |
| SERPINB7 | −0.002357 |
| VKORC1L1 | −0.002357 |
| NQO2 | −0.002358 |
| TMEM26 | −0.002359 |
| HGSNAT | −0.002360 |
| PBDC1 | −0.002361 |
| ART1 | −0.002361 |
| RPS23 | −0.002362 |
| BLOC1S1 | −0.002363 |
| ZNF467 | −0.002363 |
| TOR1AIP1 | −0.002364 |
| FCRL2 | −0.002367 |
| ECE1 | −0.002367 |
| ARF3 | −0.002367 |
| ADD1 | −0.002367 |
| TREML4 | −0.002369 |
| SNRK | −0.002369 |
| FLOT2 | −0.002370 |
| SERPINA2 | −0.002373 |
| CNR1 | −0.002373 |
| PLOD1 | −0.002373 |
| PIGB | −0.002374 |
| TREH | −0.002375 |
| TNFAIP8 | −0.002377 |
| RAB9A | −0.002380 |
| SP140L | −0.002380 |
| RNPEP | −0.002382 |
| CCR8 | −0.002382 |
| KPNA3 | −0.002382 |
| FIBCD1 | −0.002382 |
| SRSF8 | −0.002383 |
| SLK | −0.002386 |
| LCK | −0.002387 |
| NR5A2 | −0.002388 |
| HIBADH | −0.002388 |
| ETV3 | −0.002389 |
| NUP210L | −0.002389 |
| CNN3 | −0.002390 |
| TNFRSF11B | −0.002390 |
| RNF141 | −0.002390 |
| PDE5A | −0.002391 |
| PODXL | −0.002392 |
| TUSC1 | −0.002392 |
| CCDC36 | −0.002392 |
| ANKRD44 | −0.002396 |
| NAA60 | −0.002397 |
| REPS2 | −0.002398 |
| POLR2C | −0.002399 |
| RPL13 | −0.002399 |
| KRT78 | −0.002399 |
| CCDC92 | −0.002401 |
| PLEKHA2 | −0.002401 |
| KCNK1 | −0.002401 |
| C11orf68 | −0.002404 |
| RFTN2 | −0.002404 |
| SBSPON | −0.002405 |
| LIPM | −0.002405 |
| SPINK13 | −0.002406 |
| EGLN2 | −0.002408 |
| DGCR2 | −0.002410 |
| DCBLD1 | −0.002411 |
| C8A | −0.002411 |
| TMPRSS11E | −0.002411 |
| DTD2 | −0.002414 |
| SCAMP4 | −0.002415 |
| ANAPC16 | −0.002417 |
| NEBL | −0.002418 |
| PPP2R3A | −0.002418 |
| IDI1 | −0.002421 |
| SLC27A3 | −0.002423 |
| RAB7A | −0.002423 |
| CTNS | −0.002424 |
| CTNND1 | −0.002424 |
| SMIM5 | −0.002426 |
| MAPK15 | −0.002427 |
| RPL28 | −0.002427 |
| OSBPL11 | −0.002427 |
| RELL1 | −0.002428 |
| TMEM231 | −0.002432 |
| ZMYND10 | −0.002434 |
| CCL3L3 | −0.002436 |
| RELA | −0.002438 |
| ATP8B3 | −0.002439 |
| CADM1 | −0.002440 |
| PCBD1 | −0.002440 |
| AIFM2 | −0.002440 |
| IER5 | −0.002441 |
| OS9 | −0.002442 |
| EIF5A | −0.002444 |
| SERINC1 | −0.002446 |
| ZNF90 | −0.002446 |
| ATP10D | −0.002446 |
| TAF13 | −0.002447 |
| IFNAR2 | −0.002447 |
| IVD | −0.002449 |
| ACTN1 | −0.002450 |
| SNX10 | −0.002452 |
| PDE6B | −0.002452 |
| MMP23B | −0.002452 |
| TMCO2 | −0.002453 |
| TMPRSS7 | −0.002455 |
| C6orf106 | −0.002457 |
| GK | −0.002458 |
| RASSF7 | −0.002459 |
| DGCR6 | −0.002464 |
| FBP2 | −0.002464 |
| SUSD5 | −0.002466 |
| IMPDH1 | −0.002466 |
| SLC2A6 | −0.002467 |
| ITGB1 | −0.002468 |
| ALCAM | −0.002468 |
| PPTC7 | −0.002472 |
| TSGA10IP | −0.002474 |
| ASPN | −0.002474 |
| SCN9A | −0.002475 |
| MYO5B | −0.002475 |
| EVI5 | −0.002476 |
| AFAP1L2 | −0.002480 |
| MCAM | −0.002481 |
| HIC1 | −0.002483 |
| PLCL1 | −0.002486 |
| CTRB2 | −0.002486 |
| HTATEP2 | −0.002487 |
| FASN | −0.002489 |
| FDX1 | −0.002491 |
| KLHL21 | −0.002492 |
| LMO7DN | −0.002493 |
| KLF13 | −0.002493 |
| EIF6 | −0.002494 |
| FAM20C | −0.002494 |
| MTHFR | −0.002496 |
| PGD | −0.002497 |
| FAM189A1 | −0.002499 |
| ST3GAL4 | −0.002501 |
| ARHGAP8 | −0.002502 |
| CYTH3 | −0.002503 |
| SEMA4B | −0.002504 |
| SEL1L3 | −0.002504 |
| EIF4E2 | −0.002506 |
| AKTIP | −0.002508 |
| MZB1 | −0.002508 |
| NFE4 | −0.002509 |
| AMIGO1 | −0.002510 |
| FGGY | −0.002511 |
| COL4A3BP | −0.002513 |
| BRK1 | −0.002514 |
| CRAT | −0.002515 |
| RBP5 | −0.002515 |
| GABRB2 | −0.002518 |
| OLFM1 | −0.002519 |
| NCOR2 | −0.002519 |
| TOLLIP | −0.002519 |
| DDAH1 | −0.002520 |
| PWWP2B | −0.002522 |
| SAXO2 | −0.002522 |
| ATP6V0C | −0.002524 |
| SUSD1 | −0.002526 |
| MYO9A | −0.002529 |
| SLC25A1 | −0.002529 |
| C11orf91 | −0.002532 |
| KCNAB2 | −0.002532 |
| DHX32 | −0.002532 |
| DAGLB | −0.002532 |
| PLSCR1 | −0.002534 |
| TMEM62 | −0.002534 |
| HAUS4 | −0.002535 |
| GLB1L | −0.002536 |
| NOP10 | −0.002538 |
| ACOX3 | −0.002540 |
| GHITM | −0.002544 |
| JAK2 | −0.002544 |
| LAMA5 | −0.002547 |
| ASGR2 | −0.002547 |
| ADAM17 | −0.002548 |
| FSTL1 | −0.002550 |
| GBA | −0.002551 |
| TMEM256 | −0.002551 |
| FAM228A | −0.002555 |
| ARHGDIA | −0.002555 |
| PROSC | −0.002556 |
| GNAQ | −0.002557 |
| TNFRSF4 | −0.002557 |
| MYO10 | −0.002557 |
| HOXA4 | −0.002558 |
| STON1 | −0.002558 |
| KRT23 | −0.002559 |
| HORMAD1 | −0.002559 |
| SGSH | −0.002560 |
| SNX3 | −0.002561 |
| USB1 | −0.002562 |
| NOB1 | −0.002562 |
| CHST14 | −0.002563 |
| SMIM22 | −0.002565 |
| DOCK1 | −0.002568 |
| PACS1 | −0.002569 |
| NRG2 | −0.002570 |
| CRLF1 | −0.002570 |
| PITPNA | −0.002570 |
| TRPC3 | −0.002573 |
| CHMP4B | −0.002574 |
| TMEM50A | −0.002574 |
| PCYT2 | −0.002575 |
| SPHK1 | −0.002576 |
| SLAIN2 | −0.002580 |
| INTS5 | −0.002582 |
| LYNX1 | −0.002583 |
| C15orf57 | −0.002584 |
| PNRC1 | −0.002586 |
| HBM | −0.002587 |
| HK1 | −0.002588 |
| LSM10 | −0.002589 |
| VAV3 | −0.002589 |
| BCAP29 | −0.002593 |
| SLFN14 | −0.002593 |
| F2R | −0.002594 |
| PK3C2B | −0.002594 |
| ZNF728 | −0.002596 |
| NAV3 | −0.002597 |
| PRG2 | −0.002597 |
| VPS11 | −0.002600 |
| BAG1 | −0.002601 |
| LNX2 | −0.002601 |
| SPNS1 | −0.002603 |
| CPPED1 | −0.002604 |
| RAB29 | −0.002605 |
| IL4I1 | −0.002607 |
| DYX1C1 | −0.002607 |
| KIAA1191 | −0.002611 |
| RAB5B | −0.002611 |
| PDLIM3 | −0.002615 |
| EEF2K | −0.002616 |
| EVC2 | −0.002620 |
| ATRAID | −0.002620 |
| PERM1 | −0.002620 |
| SLC25A30 | −0.002621 |
| MMP13 | −0.002621 |
| BCL10 | −0.002621 |
| DHRS4 | −0.002624 |
| DLL4 | −0.002625 |
| TRIM47 | −0.002626 |
| PPP1R7 | −0.002626 |
| CDC42 | −0.002627 |
| B4GAT1 | −0.002629 |
| TMTC2 | −0.002630 |
| LDAH | −0.002632 |
| GP1BA | −0.002634 |
| RPL15 | −0.002635 |
| ZEB1 | −0.002635 |
| MERTK | −0.002637 |
| EXOC3L2 | −0.002637 |
| LMAN2 | −0.002638 |
| ZFAND5 | −0.002638 |
| ZNRF3 | −0.002638 |
| SAR1A | −0.002639 |
| SURF4 | −0.002639 |
| SORT1 | −0.002641 |
| PNPLA2 | −0.002642 |
| CCDC6 | −0.002645 |
| C7orf31 | −0.002646 |
| NCSTN | −0.002648 |
| NEGR1 | −0.002650 |
| TXN2 | −0.002651 |
| NEK6 | −0.002654 |
| RRM2B | −0.002655 |
| NT5DC1 | −0.002656 |
| HCFC1R1 | −0.002660 |
| TALDO1 | −0.002662 |
| TRNP1 | −0.002663 |
| CUX1 | −0.002664 |
| HORMAD2 | −0.002664 |
| TUSC2 | −0.002666 |
| SORCS1 | −0.002666 |
| TOMM7 | −0.002667 |
| MPZL3 | −0.002667 |
| OSCP1 | −0.002669 |
| 43525 | −0.002669 |
| KCNRG | −0.002670 |
| RNF130 | −0.002674 |
| COL4A6 | −0.002675 |
| BATF3 | −0.002675 |
| CSPG4 | −0.002676 |
| MICAL2 | −0.002679 |
| RPS9 | −0.002681 |
| NRIP1 | −0.002682 |
| EIF3CL | −0.002683 |
| SERINC3 | −0.002683 |
| NR2F1 | −0.002683 |
| FOXL1 | −0.002687 |
| S100A5 | −0.002688 |
| MAP7D3 | −0.002689 |
| TSC22D4 | −0.002690 |
| FKBP1B | −0.002690 |
| ANXA7 | −0.002690 |
| IL13 | −0.002693 |
| PTGR1 | −0.002696 |
| FOXC1 | −0.002698 |
| ARSF | −0.002700 |
| 43534 | −0.002700 |
| DIAPH1 | −0.002700 |
| TAS1R1 | −0.002701 |
| ILDR1 | −0.002701 |
| ZC3H12D | −0.002702 |
| C11orf24 | −0.002703 |
| GPRC5D | −0.002707 |
| RNF217 | −0.002707 |
| SHB | −0.002707 |
| ZNF319 | −0.002708 |
| COX14 | −0.002709 |
| MCOLN1 | −0.002710 |
| HHIPL1 | −0.002712 |
| FKBP9 | −0.002712 |
| KIAA0930 | −0.002716 |
| DOCK4 | −0.002717 |
| AMDHD1 | −0.002720 |
| DEFB4B | −0.002721 |
| CHADL | −0.002725 |
| PPP2CB | −0.002726 |
| TMEM214 | −0.002729 |
| STX8 | −0.002729 |
| SORBS1 | −0.002731 |
| SNCA | −0.002732 |
| RAB4B | −0.002734 |
| LIMK1 | −0.002734 |
| TSPAN11 | −0.002734 |
| ELF4 | −0.002734 |
| SLC4A5 | −0.002735 |
| GRB2 | −0.002736 |
| PPP1R11 | −0.002737 |
| ERP29 | −0.002738 |
| EPN3 | −0.002739 |
| RNASE3 | −0.002742 |
| EXOC3L1 | −0.002742 |
| FOXP1 | −0.002743 |
| CT45A1 | −0.002744 |
| RCVRN | −0.002744 |
| JAK3 | −0.002744 |
| MCU | −0.002745 |
| MXD4 | −0.002746 |
| DYDC1 | −0.002746 |
| TPM3 | −0.002748 |
| NLRP6 | −0.002750 |
| SARAF | −0.002751 |
| SLC41A2 | −0.002753 |
| OPRL1 | −0.002753 |
| NME5 | −0.002756 |
| STXBP2 | −0.002757 |
| DUSP23 | −0.002761 |
| FDCSP | −0.002762 |
| MSRB2 | −0.002762 |
| AMIGO2 | −0.002763 |
| P2RX4 | −0.002765 |
| SURF1 | −0.002766 |
| FGD3 | −0.002769 |
| SRD5A3 | −0.002769 |
| MEGF9 | −0.002769 |
| MUL1 | −0.002770 |
| CT45A3 | −0.002772 |
| PIGV | −0.002774 |
| ALAD | −0.002777 |
| EFNA5 | −0.002777 |
| EIF2AK4 | −0.002778 |
| IL23R | −0.002780 |
| FBX036 | −0.002785 |
| DRD1 | −0.002788 |
| NDEL1 | −0.002789 |
| SLC5A1 | −0.002789 |
| ACTC1 | −0.002790 |
| SSTR4 | −0.002791 |
| SRF | −0.002792 |
| MAGEH1 | −0.002792 |
| DUSP18 | −0.002792 |
| PIP5KL1 | −0.002792 |
| MVB12B | −0.002795 |
| MIEF2 | −0.002795 |
| LOX | −0.002798 |
| ANKS1A | −0.002801 |
| AKR1B10 | −0.002801 |
| PPA1 | −0.002802 |
| ARFIGEF10 | −0.002807 |
| ZNF486 | −0.002807 |
| TFE3 | −0.002807 |
| INAFM2 | −0.002808 |
| ALPK3 | −0.002808 |
| OSGIN2 | −0.002816 |
| NUBP1 | −0.002817 |
| CNPPD1 | −0.002817 |
| HLX | −0.002822 |
| ISCU | −0.002822 |
| STK40 | −0.002823 |
| SLC17A5 | −0.002824 |
| HHAT | −0.002825 |
| SLC39A4 | −0.002826 |
| PTGER1 | −0.002828 |
| METTL24 | −0.002830 |
| ACP2 | −0.002830 |
| KCNA10 | −0.002830 |
| ARSA | −0.002831 |
| CNNM4 | −0.002831 |
| CCDC17 | −0.002832 |
| NKX2-8 | −0.002832 |
| ANO1 | −0.002834 |
| CHMP6 | −0.002837 |
| IRF2 | −0.002840 |
| LIMS1 | −0.002841 |
| MTRR | −0.002842 |
| DNAI1 | −0.002843 |
| DAG1 | −0.002844 |
| MAGEE2 | −0.002846 |
| GLTSCR2 | −0.002846 |
| PPP1CA | −0.002848 |
| PKP3 | −0.002848 |
| HADHA | −0.002848 |
| REEP3 | −0.002851 |
| FBXL5 | −0.002851 |
| ERLEC1 | −0.002852 |
| GPAT2 | −0.002852 |
| DNM3 | −0.002852 |
| RAB17 | −0.002853 |
| CCDC28A | −0.002853 |
| GJB5 | −0.002856 |
| CCDC107 | −0.002857 |
| VAMP2 | −0.002857 |
| PINK1 | −0.002858 |
| SUOX | −0.002859 |
| PAFAH2 | −0.002860 |
| C1orf21 | −0.002860 |
| CORO1B | −0.002861 |
| CCDC13 | −0.002861 |
| SLCO1A2 | −0.002861 |
| TRIL | −0.002861 |
| PIM3 | −0.002862 |
| NLRC3 | −0.002862 |
| HADHB | −0.002865 |
| APOLD1 | −0.002865 |
| FAM188B | −0.002866 |
| SNAI2 | −0.002866 |
| ITPR2 | −0.002867 |
| BTLA | −0.002868 |
| OVCA2 | −0.002870 |
| SLAMF9 | −0.002871 |
| EHBP1L1 | −0.002872 |
| CYB561A3 | −0.002873 |
| DOLK | −0.002873 |
| TMEM167B | −0.002873 |
| QARS | −0.002873 |
| SNX7 | −0.002874 |
| EPB41L5 | −0.002874 |
| TNMD | −0.002875 |
| LIM2 | −0.002878 |
| TYR03 | −0.002880 |
| JUND | −0.002881 |
| NFE2L1 | −0.002883 |
| SLC35A1 | −0.002885 |
| TULP2 | −0.002886 |
| KCTD21 | −0.002886 |
| CYP4F11 | −0.002891 |
| PTPRK | −0.002892 |
| TNFAIP1 | −0.002895 |
| RPL3 | −0.002896 |
| SEMA4G | −0.002897 |
| MBNL2 | −0.002898 |
| PRRG3 | −0.002900 |
| PSEN2 | −0.002902 |
| NPTN | −0.002905 |
| TTC28 | −0.002907 |
| ASB13 | −0.002908 |
| HMGCL | −0.002908 |
| SREBF1 | −0.002910 |
| XKR3 | −0.002910 |
| KAT2B | −0.002913 |
| NCKAP5L | −0.002916 |
| RDH10 | −0.002917 |
| CAPZB | −0.002917 |
| ZFP92 | −0.002917 |
| ARAF | −0.002918 |
| SH3BP1 | −0.002918 |
| TNIP2 | −0.002919 |
| ITGB3 | −0.002920 |
| RPLP1 | −0.002920 |
| TEC | −0.002920 |
| LPAR6 | −0.002921 |
| BMP1 | −0.002922 |
| SIRT4 | −0.002925 |
| DDX60L | −0.002927 |
| ALG1 | −0.002928 |
| PLXNA2 | −0.002930 |
| ABHD12B | −0.002933 |
| IDO2 | −0.002933 |
| ARAP1 | −0.002934 |
| C20orf194 | −0.002935 |
| COMMD1 | −0.002935 |
| RGS9BP | −0.002936 |
| DNAAF3 | −0.002937 |
| PLA2G4C | −0.002938 |
| SERPINB11 | −0.002938 |
| PARP8 | −0.002940 |
| HSD17B12 | −0.002941 |
| MICB | −0.002943 |
| C3orf52 | −0.002943 |
| MTMR11 | −0.002947 |
| ZNF593 | −0.002947 |
| LRCH1 | −0.002948 |
| BCL2L12 | −0.002950 |
| NRBF2 | −0.002951 |
| SNX8 | −0.002952 |
| MAP3K11 | −0.002952 |
| RGS6 | −0.002954 |
| KCNA4 | −0.002955 |
| CYFIP1 | −0.002958 |
| LRRC29 | −0.002963 |
| GSTT2 | −0.002964 |
| OGDH | −0.002965 |
| CBR1 | −0.002967 |
| CCDC153 | −0.002967 |
| CCDC103 | −0.002969 |
| CROT | −0.002971 |
| ARRB2 | −0.002971 |
| WNT5A | −0.002973 |
| PPP1R32 | −0.002973 |
| ACADSB | −0.002978 |
| PSKH1 | −0.002978 |
| SERPINH1 | −0.002986 |
| SRI | −0.002986 |
| SPRED2 | −0.002986 |
| ADD3 | −0.002987 |
| GPR85 | −0.002988 |
| ACTRT3 | −0.002988 |
| PDCD6IP | −0.002990 |
| LGR6 | −0.002991 |
| PGAM4 | −0.002993 |
| CLRN3 | −0.002993 |
| NUDT7 | −0.002993 |
| PNOC | −0.002996 |
| GPR55 | −0.003000 |
| PSD4 | −0.003003 |
| CAB39 | −0.003003 |
| PSME1 | −0.003003 |
| CLDN4 | −0.003004 |
| RPS6KA4 | −0.003006 |
| MAST4 | −0.003007 |
| MCTP2 | −0.003008 |
| IGFLR1 | −0.003013 |
| GGA2 | −0.003014 |
| NFKBIE | −0.003017 |
| HIPK3 | −0.003024 |
| TMEM30A | −0.003025 |
| LASP1 | −0.003026 |
| GJB2 | −0.003026 |
| SYTL3 | −0.003027 |
| CARD9 | −0.003030 |
| GUCD1 | −0.003030 |
| MAP4K1 | −0.003032 |
| PEF1 | −0.003034 |
| PRKAR2A | −0.003036 |
| UGT2B17 | −0.003036 |
| ATP6V1F | −0.003039 |
| STIM1 | −0.003039 |
| IZUMO2 | −0.003042 |
| IL2RA | −0.003043 |
| STOML1 | −0.003047 |
| MIF4GD | −0.003047 |
| PRKCA | −0.003048 |
| RPS14 | −0.003051 |
| ISG15 | −0.003054 |
| SLC25A24 | −0.003055 |
| GPRIN3 | −0.003059 |
| PDE3A | −0.003059 |
| FAM174A | −0.003060 |
| C22orf31 | −0.003060 |
| RAB1B | −0.003061 |
| PTK2B | −0.003065 |
| CD28 | −0.003067 |
| MFSD6L | −0.003068 |
| DOCK5 | −0.003070 |
| COL23A1 | −0.003075 |
| TCEAL7 | −0.003076 |
| PKHD1 | −0.003078 |
| SLC31A1 | −0.003084 |
| CLCNKB | −0.003086 |
| NOTCH3 | −0.003088 |
| CCDC184 | −0.003089 |
| FKBP5 | −0.003089 |
| PLA2G2C | −0.003091 |
| BTNL3 | −0.003096 |
| BAAT | −0.003102 |
| NAALADL2 | −0.003103 |
| DNAJB12 | −0.003106 |
| RAPGEF1 | −0.003106 |
| ARRDC3 | −0.003107 |
| ELMO1 | −0.003107 |
| ARMT1 | −0.003110 |
| GPR137B | −0.003111 |
| CFAP43 | −0.003113 |
| ACOT2 | −0.003116 |
| DSE | −0.003116 |
| TIMP1 | −0.003117 |
| FGF18 | −0.003117 |
| FCAMR | −0.003120 |
| MED15 | −0.003120 |
| MYOM2 | −0.003121 |
| IDNK | −0.003123 |
| ZNF385A | −0.003124 |
| CDK2AP2 | −0.003125 |
| MCFD2 | −0.003127 |
| FGD6 | −0.003128 |
| PPP1R9B | −0.003129 |
| HERPUD1 | −0.003130 |
| COBLL1 | −0.003130 |
| EIF4E3 | −0.003139 |
| TIGAR | −0.003141 |
| CLDN10 | −0.003142 |
| RPL14 | −0.003143 |
| SMIM10L2A | −0.003145 |
| CYTH1 | −0.003148 |
| DOCK6 | −0.003148 |
| LRRC55 | −0.003148 |
| ARSB | −0.003153 |
| TCIRG1 | −0.003157 |
| WBP2 | −0.003161 |
| COMP | −0.003161 |
| NUDT16 | −0.003163 |
| DCBLD2 | −0.003164 |
| C11orf86 | −0.003165 |
| ART5 | −0.003165 |
| DNAH5 | −0.003165 |
| SHANK2 | −0.003168 |
| KCTD10 | −0.003169 |
| RHOU | −0.003170 |
| SCP2 | −0.003172 |
| CCNDBP1 | −0.003174 |
| HIPK4 | −0.003174 |
| XYLT2 | −0.003176 |
| MID2 | −0.003177 |
| ORMDL2 | −0.003182 |
| ASZ1 | −0.003185 |
| BFAR | −0.003186 |
| RNF19B | −0.003188 |
| CNTN3 | −0.003189 |
| LAMP2 | −0.003190 |
| CERS2 | −0.003190 |
| FAHD2B | −0.003193 |
| TCTA | −0.003194 |
| GSTA5 | −0.003194 |
| HIP1 | −0.003195 |
| DUSP2 | −0.003195 |
| BCAR1 | −0.003195 |
| TM4SF18 | −0.003196 |
| TMEM101 | −0.003196 |
| ERICH4 | −0.003197 |
| GPR174 | −0.003204 |
| DMRTA1 | −0.003206 |
| NFE2L2 | −0.003206 |
| RHOQ | −0.003206 |
| TLCD1 | −0.003208 |
| B3GNT2 | −0.003208 |
| PKHD1L1 | −0.003210 |
| RNASET2 | −0.003211 |
| DPY19L1 | −0.003213 |
| ARRDC4 | −0.003214 |
| PIK3R1 | −0.003214 |
| C12orf54 | −0.003215 |
| HIVEP2 | −0.003216 |
| LAMTOR1 | −0.003218 |
| INCA1 | −0.003218 |
| SH3TC1 | −0.003220 |
| DST | −0.003221 |
| WLS | −0.003221 |
| SPAG7 | −0.003222 |
| CYP7B1 | −0.003222 |
| IL12B | −0.003223 |
| C11orf16 | −0.003230 |
| SMURF1 | −0.003231 |
| CT83 | −0.003232 |
| FANK1 | −0.003234 |
| PTTG1IP | −0.003235 |
| BPIFA2 | −0.003241 |
| CALHM2 | −0.003241 |
| SMARCD3 | −0.003244 |
| HK2 | −0.003244 |
| MAPKAPK2 | −0.003246 |
| ERF | −0.003255 |
| CHRFAM7A | −0.003257 |
| ADK | −0.003258 |
| LTA | −0.003261 |
| TMED5 | −0.003263 |
| KCNMB4 | −0.003264 |
| ZBTB7A | −0.003269 |
| MKX | −0.003274 |
| NMI | −0.003276 |
| CNRIP1 | −0.003276 |
| PACSIN2 | −0.003278 |
| CASP8 | −0.003280 |
| NPY5R | −0.003280 |
| KLF10 | −0.003282 |
| KAZALD1 | −0.003285 |
| TNFRSF19 | −0.003288 |
| LRRC8A | −0.003290 |
| HS3ST3B1 | −0.003292 |
| GRIN2A | −0.003293 |
| CHST11 | −0.003293 |
| PPARGC1B | −0.003294 |
| PTPN5 | −0.003294 |
| RABAC1 | −0.003296 |
| XIRP1 | −0.003296 |
| NOV | −0.003297 |
| ATP6V1E1 | −0.003302 |
| CAMK2N1 | −0.003304 |
| JAGN1 | −0.003305 |
| HEYL | −0.003305 |
| FLG | −0.003305 |
| XBP1 | −0.003307 |
| PEPD | −0.003307 |
| IFFO1 | −0.003311 |
| PHF11 | −0.003314 |
| PMVK | −0.003315 |
| DACT2 | −0.003315 |
| SPATA4 | −0.003317 |
| SNX24 | −0.003318 |
| SPTSSA | −0.003320 |
| FAM187A | −0.003321 |
| SYBU | −0.003323 |
| ECT2L | −0.003324 |
| ST7 | −0.003326 |
| TANGO2 | −0.003330 |
| KLK6 | −0.003332 |
| SYDE2 | −0.003332 |
| NIPAL4 | −0.003334 |
| C21orf33 | −0.003334 |
| PRLR | −0.003335 |
| ZBP1 | −0.003335 |
| BRE | −0.003337 |
| TNK1 | −0.003338 |
| FAM71A | −0.003339 |
| CRLS1 | −0.003342 |
| UTF1 | −0.003342 |
| ABCG1 | −0.003344 |
| TMEM245 | −0.003344 |
| C2orf73 | −0.003345 |
| NUMB | −0.003345 |
| M6PR | −0.003346 |
| DSG2 | −0.003350 |
| MS4A3 | −0.003350 |
| TANC1 | −0.003351 |
| FGF1 | −0.003351 |
| DMD | −0.003351 |
| SH3RF1 | −0.003353 |
| MEF2D | −0.003354 |
| CAPZA2 | −0.003356 |
| SAT2 | −0.003357 |
| HSD17B4 | −0.003358 |
| BNC2 | −0.003359 |
| EOMES | −0.003360 |
| CAB | −0.003360 |
| ZNF106 | −0.003364 |
| CLEC17A | −0.003364 |
| CILP | −0.003364 |
| CHMP3 | −0.003364 |
| EXPH5 | −0.003365 |
| ARHGEF17 | −0.003367 |
| PRDX1 | −0.003367 |
| HOXA5 | −0.003368 |
| FIS1 | −0.003369 |
| SH2B3 | −0.003369 |
| SIPA1 | −0.003370 |
| PKD1L2 | −0.003370 |
| BTBD19 | −0.003371 |
| TMEM127 | −0.003372 |
| STAC2 | −0.003372 |
| PCBP1 | −0.003372 |
| MREG | −0.003373 |
| PRSS16 | −0.003376 |
| FOXJ1 | −0.003376 |
| ADGRA1 | −0.003377 |
| CALCOCO2 | −0.003382 |
| GSTM4 | −0.003383 |
| PVALB | −0.003383 |
| NDUFAF3 | −0.003384 |
| CBLN4 | −0.003385 |
| LTBP3 | −0.003388 |
| MPDU1 | −0.003389 |
| HHEX | −0.003389 |
| LIMA1 | −0.003391 |
| DUXA | −0.003393 |
| C1orf53 | −0.003395 |
| ETF1 | −0.003396 |
| HERC5 | −0.003397 |
| TCTN3 | −0.003397 |
| ACAT1 | −0.003399 |
| ELM03 | −0.003400 |
| HOMER1 | −0.003400 |
| TTC39A | −0.003400 |
| KIAA1161 | −0.003401 |
| KRT8 | −0.003403 |
| HDDC2 | −0.003404 |
| KLRG1 | −0.003405 |
| OAF | −0.003405 |
| TGIF2 | −0.003407 |
| FNDC1 | −0.003411 |
| ABHD4 | −0.003413 |
| PTPN1 | −0.003415 |
| COL4A2 | −0.003416 |
| TEF | −0.003418 |
| BTN2A2 | −0.003419 |
| FAM183A | −0.003419 |
| CLEC4G | −0.003420 |
| TMEM110 | −0.003422 |
| LHX6 | −0.003423 |
| NATD1 | −0.003425 |
| SVIL | −0.003426 |
| VWA2 | −0.003427 |
| ELF3 | −0.003429 |
| SLC6A16 | −0.003430 |
| SHC1 | −0.003435 |
| TMEM179B | −0.003436 |
| CNR2 | −0.003437 |
| FAM214B | −0.003439 |
| ADAM33 | −0.003439 |
| PROM2 | −0.003440 |
| PHF24 | −0.003441 |
| JAK1 | −0.003443 |
| EEF1A1 | −0.003443 |
| DESI1 | −0.003443 |
| PPDPF | −0.003445 |
| GCNT2 | −0.003446 |
| SIGLEC10 | −0.003447 |
| PTN | −0.003449 |
| KCNJ1 | −0.003450 |
| SFT2D1 | −0.003452 |
| GRAP2 | −0.003453 |
| CASC1 | −0.003455 |
| RAB13 | −0.003459 |
| TDGF1 | −0.003460 |
| AMFR | −0.003463 |
| DPH3 | −0.003463 |
| CYP2B6 | −0.003467 |
| TRIM56 | −0.003469 |
| MBIP | −0.003470 |
| GSDMD | −0.003470 |
| FDFT1 | −0.003471 |
| BTBD9 | −0.003472 |
| KITLG | −0.003474 |
| PTPRZ1 | −0.003476 |
| CDCP1 | −0.003476 |
| IFI44 | −0.003479 |
| FAM13C | −0.003479 |
| CDC42EP4 | −0.003485 |
| IL2 | −0.003489 |
| PPP4R4 | −0.003491 |
| BET1L | −0.003493 |
| HMOX2 | −0.003496 |
| SLC35A4 | −0.003498 |
| PYGM | −0.003498 |
| NCAM2 | −0.003499 |
| EMC7 | −0.003499 |
| PAMR1 | −0.003499 |
| RASGEF1A | −0.003500 |
| SESN2 | −0.003501 |
| ZDHHC9 | −0.003501 |
| LOXL4 | −0.003503 |
| FNDC3B | −0.003504 |
| SLC2A4 | −0.003509 |
| MEGF6 | −0.003511 |
| ZDHHC16 | −0.003511 |
| GFRA2 | −0.003511 |
| AKNA | −0.003513 |
| CRISPLD2 | −0.003513 |
| ESRP2 | −0.003515 |
| C17orf97 | −0.003516 |
| BMP7 | −0.003517 |
| ACOT4 | −0.003517 |
| RHBDL2 | −0.003523 |
| SNX17 | −0.003523 |
| FAM26F | −0.003525 |
| IDH1 | −0.003527 |
| HS1BP3 | −0.003529 |
| MAS1L | −0.003530 |
| DMTN | −0.003530 |
| MDS2 | −0.003533 |
| IMPA2 | −0.003543 |
| VWA1 | −0.003544 |
| GPR153 | −0.003545 |
| RNF122 | −0.003545 |
| LAMB1 | −0.003548 |
| TSHZ3 | −0.003551 |
| TMEM53 | −0.003552 |
| TMSB10 | −0.003554 |
| CAPN12 | −0.003556 |
| KIAA0319L | −0.003556 |
| B3GNT9 | −0.003556 |
| CACNG4 | −0.003560 |
| MICALCL | −0.003568 |
| SPEF1 | −0.003568 |
| BMPR2 | −0.003572 |
| PHLDA2 | −0.003576 |
| GPR160 | −0.003577 |
| FLII | −0.003578 |
| SPRR2D | −0.003578 |
| FHL3 | −0.003578 |
| FAM109B | −0.003580 |
| ANO10 | −0.003580 |
| NAT2 | −0.003582 |
| GABARAP | −0.003583 |
| CORO1C | −0.003584 |
| CDK6 | −0.003588 |
| TBC1D9 | −0.003591 |
| CHST9 | −0.003592 |
| RAPH1 | −0.003593 |
| DNASE2 | −0.003594 |
| APELA | −0.003594 |
| IRS1 | −0.003598 |
| HSDL2 | −0.003599 |
| UBAP1 | −0.003600 |
| CDKN1A | −0.003602 |
| ADORA1 | −0.003604 |
| SDC2 | −0.003606 |
| CAPN3 | −0.003609 |
| DISP1 | −0.003609 |
| ADH5 | −0.003609 |
| UPK1B | −0.003610 |
| CTBS | −0.003611 |
| METTL21C | −0.003613 |
| GANC | −0.003615 |
| HRH2 | −0.003616 |
| NOVA2 | −0.003617 |
| OSBPL1A | −0.003620 |
| ZG16B | −0.003620 |
| REN | −0.003620 |
| DNAJC3 | −0.003624 |
| CCDC71 | −0.003624 |
| SLFN12L | −0.003625 |
| STARD10 | −0.003626 |
| SPATAI2 | −0.003628 |
| TOBI | −0.003628 |
| WWC1 | −0.003630 |
| TSC22D1 | −0.003630 |
| PLBD2 | −0.003632 |
| GALNT3 | −0.003635 |
| NODI | −0.003637 |
| CFL2 | −0.003641 |
| IGF2 | −0.003644 |
| NFIL3 | −0.003644 |
| CFAP53 | −0.003645 |
| STAC3 | −0.003645 |
| LPP | −0.003646 |
| TSPAN19 | −0.003646 |
| TSPAN2 | −0.003650 |
| SULT1C2 | −0.003654 |
| PPM1F | −0.003656 |
| PAPLN | −0.003658 |
| HPS5 | −0.003658 |
| ZP2 | −0.003658 |
| ACVR1 | −0.003658 |
| CTSK | −0.003660 |
| ARL2BP | −0.003661 |
| IKBKE | −0.003663 |
| RNF181 | −0.003666 |
| DLEU7 | −0.003667 |
| PTP4A1 | −0.003670 |
| SLC27A1 | −0.003670 |
| SPR | −0.003670 |
| PARP14 | −0.003671 |
| PKIG | −0.003671 |
| TBC1D22A | −0.003671 |
| TNNC2 | −0.003672 |
| SAMD3 | −0.003674 |
| MYOZ2 | −0.003675 |
| KCNJ12 | −0.003675 |
| ACTBL2 | −0.003676 |
| EPP1N | −0.003678 |
| INF2 | −0.003678 |
| RPL29 | −0.003678 |
| FLT3LG | −0.003680 |
| SYNM | −0.003680 |
| WNT5B | −0.003681 |
| RAP1GAP | −0.003681 |
| WDR1 | −0.003688 |
| APRT | −0.003689 |
| GPR108 | −0.003690 |
| SH3TC2 | −0.003693 |
| SPINT1 | −0.003693 |
| ZNF541 | −0.003694 |
| CCDC115 | −0.003697 |
| CYB5D2 | −0.003698 |
| NUDT8 | −0.003699 |
| PRRG2 | −0.003699 |
| TAP2 | −0.003701 |
| C17orf107 | −0.003701 |
| TRPM1 | −0.003702 |
| PDGFRB | −0.003702 |
| RAB8B | −0.003703 |
| PEX11G | −0.003703 |
| CAMK1 | −0.003706 |
| PMEPA1 | −0.003708 |
| MFSD1 | −0.003708 |
| CAPS | −0.003711 |
| VWA7 | −0.003712 |
| DTX2 | −0.003713 |
| OTOA | −0.003713 |
| DNAJB4 | −0.003714 |
| TPSG1 | −0.003715 |
| BCAP31 | −0.003719 |
| PKP1 | −0.003722 |
| PPP1R13L | −0.003724 |
| COL21A1 | −0.003725 |
| ARMC7 | −0.003728 |
| TSPAN31 | −0.003730 |
| BINI | −0.003731 |
| CRYZ | −0.003734 |
| GNS | −0.003737 |
| GRAPL | −0.003738 |
| IFI27L2 | −0.003740 |
| SIGLEC16 | −0.003742 |
| PRR15L | −0.003745 |
| MAP1LC3B | −0.003748 |
| SNX2 | −0.003748 |
| TMEM45B | −0.003753 |
| MYL6 | −0.003753 |
| PRCP | −0.003755 |
| AP1S3 | −0.003757 |
| BTN3A1 | −0.003759 |
| CALD1 | −0.003760 |
| NR2F2 | −0.003760 |
| COPS7A | −0.003768 |
| SLFN11 | −0.003770 |
| COL26A1 | −0.003770 |
| AIM2 | −0.003771 |
| NEIL2 | −0.003771 |
| STK17A | −0.003772 |
| CHMP2A | −0.003775 |
| PDE1C | −0.003777 |
| SLC12A3 | −0.003780 |
| HDAC7 | −0.003781 |
| DGKG | −0.003783 |
| MTMR14 | −0.003784 |
| SUMF2 | −0.003786 |
| MPST | −0.003787 |
| TGM1 | −0.003787 |
| TMEM200A | −0.003787 |
| DSEL | −0.003797 |
| BCO1 | −0.003798 |
| TMEM45A | −0.003799 |
| FNIP2 | −0.003801 |
| GPR84 | −0.003802 |
| GLIPR1L2 | −0.003803 |
| RARB | −0.003804 |
| KRT32 | −0.003805 |
| DCC | −0.003806 |
| CDH17 | −0.003808 |
| NMB | −0.003808 |
| RASSF3 | −0.003810 |
| IL10RB | −0.003811 |
| HSD17B14 | −0.003812 |
| SLFN12 | −0.003815 |
| TBC1D10A | −0.003817 |
| TTC23 | −0.003817 |
| TAP1 | −0.003819 |
| UNC13B | −0.003822 |
| CAP1 | −0.003825 |
| KRCC1 | −0.003825 |
| PDGFRL | −0.003829 |
| WIPI1 | −0.003831 |
| ARF4 | −0.003836 |
| TMEM115 | −0.003838 |
| TGOLN2 | −0.003839 |
| NHSL2 | −0.003839 |
| PLCE1 | −0.003842 |
| PPM1K | −0.003843 |
| MOB3A | −0.003844 |
| PPT1 | −0.003845 |
| TCTEX1D4 | −0.003846 |
| CD200R1 | −0.003847 |
| FBN1 | −0.003848 |
| DEF6 | −0.003851 |
| C1orf54 | −0.003857 |
| TNFRSF17 | −0.003860 |
| CAPNS2 | −0.003861 |
| FAM114A1 | −0.003862 |
| ABHD5 | −0.003864 |
| NFIX | −0.003867 |
| CORO1A | −0.003867 |
| LAMC1 | −0.003868 |
| PARD3B | −0.003870 |
| PI4K2A | −0.003874 |
| LRRFIP1 | −0.003876 |
| NLRX1 | −0.003876 |
| ECHI | −0.003883 |
| GLMP | −0.003884 |
| OSTM1 | −0.003886 |
| FUCA2 | −0.003887 |
| KRTCAP3 | −0.003888 |
| PAPSS1 | −0.003888 |
| GDF7 | −0.003889 |
| TOM1L2 | −0.003892 |
| LHFP | −0.003895 |
| RGS22 | −0.003897 |
| RHBDF2 | −0.003897 |
| FRY | −0.003898 |
| FOXP2 | −0.003900 |
| KISSI | −0.003900 |
| NABP1 | −0.003902 |
| MGAM | −0.003904 |
| MYH10 | −0.003904 |
| ODF3B | −0.003904 |
| FAR2 | −0.003905 |
| LUZP1 | −0.003906 |
| SLC35F6 | −0.003906 |
| ARL8B | −0.003911 |
| SLC20A2 | −0.003911 |
| ACSM5 | −0.003914 |
| CD47 | −0.003914 |
| RND3 | −0.003915 |
| CLIC1 | −0.003921 |
| XAF1 | −0.003923 |
| LRPAP1 | −0.003924 |
| CAPN11 | −0.003925 |
| TRANK1 | −0.003925 |
| COQ10B | −0.003927 |
| SLC5A4 | −0.003936 |
| CLDN6 | −0.003936 |
| UBC | −0.003937 |
| C15orf39 | −0.003940 |
| LHX9 | −0.003942 |
| LMBRD1 | −0.003943 |
| C2orf74 | −0.003946 |
| KCNQ3 | −0.003947 |
| C11orf52 | −0.003947 |
| GDE1 | −0.003949 |
| RHBDD2 | −0.003951 |
| CXorf65 | −0.003953 |
| CMTM6 | −0.003956 |
| NYNRIN | −0.003957 |
| MS4A1 | −0.003962 |
| DNAH11 | −0.003964 |
| GLUD1 | −0.003967 |
| ADGRG2 | −0.003968 |
| ATG7 | −0.003969 |
| ADGRA2 | −0.003972 |
| NEU1 | −0.003972 |
| MYL4 | −0.003972 |
| RCAN1 | −0.003973 |
| PFN1 | −0.003980 |
| C1QTNF9 | −0.003983 |
| KIAA2012 | −0.003985 |
| CCDC160 | −0.003985 |
| PTGER3 | −0.003988 |
| SPACA3 | −0.003988 |
| DECR1 | −0.003988 |
| OCEL1 | −0.003989 |
| RAG1 | −0.003993 |
| PLEKHM1 | −0.003993 |
| AKR1B1 | −0.003994 |
| KIAA1671 | −0.003994 |
| CDH13 | −0.003995 |
| LY6K | −0.003995 |
| COL6A3 | −0.003995 |
| ACAP1 | −0.003996 |
| TRIOBP | −0.003996 |
| ABHD15 | −0.003996 |
| IL17D | −0.003997 |
| RAB5C | −0.003998 |
| NBPF10 | −0.003998 |
| ANGPTL2 | −0.004000 |
| FCHO2 | −0.004002 |
| F11R | −0.004004 |
| IL24 | −0.004005 |
| POLR2L | −0.004005 |
| DUOXA2 | −0.004006 |
| XG | −0.004006 |
| TSPAN14 | −0.004006 |
| GGCX | −0.004010 |
| GRINA | −0.004010 |
| PNPLA6 | −0.004011 |
| RASGRP3 | −0.004013 |
| TIMP4 | −0.004016 |
| MYBPC2 | −0.004019 |
| TRIM38 | −0.004020 |
| TEKT3 | −0.004021 |
| DENND2D | −0.004022 |
| C4orf3 | −0.004023 |
| ECHDC1 | −0.004029 |
| ARHGAP28 | −0.004030 |
| ACADS | −0.004030 |
| DHRS12 | −0.004030 |
| HTR7 | −0.004034 |
| LHPP | −0.004036 |
| PODNL1 | −0.004037 |
| TOB2 | −0.004037 |
| IFIH1 | −0.004039 |
| SCML4 | −0.004039 |
| MAB21L3 | −0.004049 |
| PDK2 | −0.004053 |
| ST6GALNAC3 | −0.004054 |
| DSC3 | −0.004055 |
| C5orf49 | −0.004058 |
| MAFB | −0.004060 |
| ZDHHC1 | −0.004065 |
| ARHGAP1 | −0.004068 |
| BCKDHA | −0.004069 |
| FAM109A | −0.004071 |
| ADGRG4 | −0.004071 |
| RSPH1 | −0.004075 |
| SDSL | −0.004077 |
| ASPH | −0.004078 |
| BTG2 | −0.004079 |
| BICC1 | −0.004084 |
| ABHD14A | −0.004086 |
| SLC35D2 | −0.004089 |
| SCPEP1 | −0.004090 |
| B4GALNT3 | −0.004094 |
| CHST6 | −0.004096 |
| HPS6 | −0.004101 |
| KCNIP1 | −0.004101 |
| MCTP1 | −0.004105 |
| NEDD4L | −0.004108 |
| USP54 | −0.004111 |
| SOAT1 | −0.004114 |
| TNIK | −0.004114 |
| CTNNA1 | −0.004115 |
| GPR20 | −0.004119 |
| PRTN3 | −0.004120 |
| EFHB | −0.004120 |
| SLC48A1 | −0.004125 |
| CTNNB1 | −0.004127 |
| TSPAN9 | −0.004128 |
| MMP25 | −0.004129 |
| PPIL6 | −0.004131 |
| NHSL1 | −0.004132 |
| TRAF3IP3 | −0.004134 |
| NPAS2 | −0.004134 |
| SNX21 | −0.004137 |
| PLAG1 | −0.004139 |
| SNED1 | −0.004141 |
| SLC16A13 | −0.004147 |
| IQSEC1 | −0.004151 |
| SLC12A4 | −0.004152 |
| GYG2 | −0.004158 |
| RNASEK | −0.004158 |
| VPS37B | −0.004159 |
| CNGA4 | −0.004159 |
| ZBTB38 | −0.004160 |
| SKI | −0.004161 |
| SIL1 | −0.004162 |
| EMC3 | −0.004162 |
| GIPC3 | −0.004163 |
| TMCO4 | −0.004163 |
| SOWAHC | −0.004164 |
| SSFA2 | −0.004164 |
| NR1H3 | −0.004165 |
| MSRB1 | −0.004166 |
| GIPC1 | −0.004167 |
| QDPR | −0.004170 |
| EBPL | −0.004170 |
| NOS1 | −0.004170 |
| DAZAP2 | −0.004173 |
| FOXA3 | −0.004175 |
| LGI4 | −0.004176 |
| GCNT3 | −0.004177 |
| TLE4 | −0.004191 |
| POLR3H | −0.004192 |
| MOGAT1 | −0.004195 |
| SLC26A4 | −0.004196 |
| REL | −0.004196 |
| CHPT1 | −0.004196 |
| NXNL2 | −0.004199 |
| RXFP2 | −0.004200 |
| NMRK1 | −0.004202 |
| OLAH | −0.004203 |
| KIF12 | −0.004205 |
| ADAM8 | −0.004206 |
| SPAG17 | −0.004206 |
| RIPK2 | −0.004207 |
| TNFRSF21 | −0.004208 |
| ZNF597 | −0.004210 |
| CCL7 | −0.004212 |
| TPM2 | −0.004213 |
| EFR3A | −0.004218 |
| CES2 | −0.004221 |
| CTAGE8 | −0.004225 |
| SCUBE2 | −0.004228 |
| MYL12B | −0.004228 |
| ACTN4 | −0.004228 |
| CADM3 | −0.004229 |
| INPP5A | −0.004237 |
| CD19 | −0.004241 |
| ETS2 | −0.004241 |
| ANO6 | −0.004241 |
| PIEZO2 | −0.004244 |
| C8orf58 | −0.004244 |
| YPEL5 | −0.004244 |
| MUC20 | −0.004246 |
| PLEKHG1 | −0.004246 |
| LRRC4B | −0.004247 |
| MYBPH | −0.004252 |
| TSKU | −0.004255 |
| IP6K3 | −0.004255 |
| POLE4 | −0.004256 |
| TSPAN8 | −0.004257 |
| KCTD14 | −0.004260 |
| ZNF438 | −0.004261 |
| MAT2B | −0.004262 |
| DNAJB13 | −0.004264 |
| S1PR2 | −0.004265 |
| ARMC3 | −0.004268 |
| SYS1 | −0.004270 |
| TSPAN15 | −0.004273 |
| RILPL1 | −0.004274 |
| HBD | −0.004274 |
| WDR49 | −0.004278 |
| CDH3 | −0.004281 |
| SERINC2 | −0.004284 |
| ACO1 | −0.004288 |
| DCDC2B | −0.004289 |
| PCDH17 | −0.004289 |
| SULT1C4 | −0.004292 |
| ACOT9 | −0.004293 |
| ARPC2 | −0.004294 |
| SERPINE1 | −0.004294 |
| MT-ND5 | −0.004295 |
| RNF13 | −0.004295 |
| LCNL1 | −0.004303 |
| NAGPA | −0.004303 |
| KRT15 | −0.004305 |
| ERBB4 | −0.004310 |
| NOTCH4 | −0.004314 |
| KCNN3 | −0.004315 |
| ADAM15 | −0.004316 |
| EVC | −0.004317 |
| PARVG | −0.004321 |
| APOBEC3D | −0.004324 |
| TCAF2 | −0.004324 |
| HNF4A | −0.004331 |
| STX12 | −0.004333 |
| NAGLU | −0.004335 |
| KRT16 | −0.004336 |
| RBM43 | −0.004336 |
| ETV1 | −0.004340 |
| SLC25A20 | −0.004341 |
| WFDC3 | −0.004344 |
| CAI | −0.004348 |
| ARL2 | −0.004348 |
| SLC29A3 | −0.004349 |
| ADAMTS9 | −0.004349 |
| CCNYL1 | −0.004349 |
| XAGE3 | −0.004350 |
| KIF16B | −0.004352 |
| C16orf74 | −0.004355 |
| EDA | −0.004355 |
| SPARC | −0.004356 |
| TK2 | −0.004356 |
| IL17RA | −0.004357 |
| LRRC3B | −0.004357 |
| ILK | −0.004359 |
| LRP5 | −0.004361 |
| SPRR1B | −0.004364 |
| PRRX1 | −0.004366 |
| PRDX6 | −0.004368 |
| PIK3CD | −0.004369 |
| SLC2A14 | −0.004371 |
| ARC | −0.004372 |
| UPK2 | −0.004373 |
| KRT14 | −0.004376 |
| EPS8L2 | −0.004376 |
| DPEP3 | −0.004377 |
| STAP1 | −0.004378 |
| PDXK | −0.004379 |
| SIT1 | −0.004381 |
| SSTR1 | −0.004381 |
| GRTP1 | −0.004386 |
| C10orf142 | −0.004387 |
| CXCL9 | −0.004388 |
| ACHE | −0.004393 |
| TMEM63A | −0.004394 |
| ZBBX | −0.004395 |
| PLIN5 | −0.004395 |
| MAFK | −0.004397 |
| CD226 | −0.004398 |
| SEMA3D | −0.004398 |
| NPFFR1 | −0.004401 |
| KIAA1522 | −0.004402 |
| LILRB5 | −0.004403 |
| TMOD3 | −0.004405 |
| MKNK2 | −0.004406 |
| UBE2L6 | −0.004406 |
| IL9R | −0.004410 |
| BCL2L15 | −0.004414 |
| CREB3L2 | −0.004415 |
| CALCR | −0.004416 |
| CD8B | −0.004417 |
| TPPP | −0.004418 |
| TSPAN3 | −0.004419 |
| ACAA1 | −0.004420 |
| IRX2 | −0.004422 |
| RSPH4A | −0.004424 |
| OR52N4 | −0.004424 |
| GSTK1 | −0.004424 |
| PEA15 | −0.004425 |
| TMEM9B | −0.004426 |
| C16orf71 | −0.004426 |
| RNF180 | −0.004427 |
| MEIRN | −0.004428 |
| CCM2L | −0.004433 |
| ZHX2 | −0.004437 |
| GPX8 | −0.004438 |
| NCOA4 | −0.004442 |
| ITGA1 | −0.004445 |
| TLR6 | −0.004449 |
| PML | −0.004456 |
| ERAP2 | −0.004456 |
| TMEM27 | −0.004464 |
| NECAP2 | −0.004464 |
| ANGPTL4 | −0.004465 |
| EXT1 | −0.004466 |
| SNA13 | −0.004467 |
| AAED1 | −0.004468 |
| G6PD | −0.004470 |
| MCL1 | −0.004471 |
| MID1IP1 | −0.004478 |
| TBC1D8B | −0.004482 |
| NAGA | −0.004483 |
| C6orf132 | −0.004490 |
| GLUL | −0.004490 |
| UCN3 | −0.004492 |
| ETV7 | −0.004492 |
| ZMYND12 | −0.004494 |
| RAB25 | −0.004495 |
| NTM | −0.004495 |
| ATP1B2 | −0.004500 |
| FCRLA | −0.004500 |
| SMIM14 | −0.004504 |
| IER2 | −0.004505 |
| SAT1 | −0.004509 |
| DRGX | −0.004510 |
| ARHGAP10 | −0.004510 |
| CYP4V2 | −0.004514 |
| PARVA | −0.004516 |
| GNG5 | −0.004516 |
| EFCAB14 | −0.004527 |
| TMEM47 | −0.004530 |
| ELMOD1 | −0.004533 |
| MSX2 | −0.004534 |
| MAPK3 | −0.004535 |
| RTP4 | −0.004537 |
| ARHGAP42 | −0.004537 |
| USP53 | −0.004539 |
| DBI | −0.004540 |
| SDCBP2 | −0.004542 |
| FBXW4 | −0.004545 |
| SCNN1A | −0.004548 |
| LGMN | −0.004549 |
| SERPINA5 | −0.004549 |
| CLDN7 | −0.004550 |
| CFLAR | −0.004556 |
| ZNF423 | −0.004556 |
| EPHX2 | −0.004557 |
| MEDAG | −0.004558 |
| UBQLNL | −0.004558 |
| SLC19A1 | −0.004560 |
| GUCY1A2 | −0.004561 |
| STAT3 | −0.004566 |
| KRT222 | −0.004566 |
| EPB41L1 | −0.004567 |
| ARAP3 | −0.004572 |
| ARRDC5 | −0.004573 |
| CCDC54 | −0.004574 |
| PRSS36 | −0.004574 |
| RRAS2 | −0.004576 |
| TDRP | −0.004577 |
| NOTCH1 | −0.004577 |
| DDX60 | −0.004579 |
| ATP1B1 | −0.004584 |
| HSPB1 | −0.004584 |
| BDKRB2 | −0.004585 |
| TRAM1L1 | −0.004588 |
| RNF149 | −0.004589 |
| NEK7 | −0.004593 |
| REP15 | −0.004594 |
| PALLD | −0.004597 |
| HTRA4 | −0.004599 |
| TUBA4A | −0.004600 |
| ADARB1 | −0.004601 |
| TRABD2B | −0.004602 |
| IGFBP7 | −0.004602 |
| AATK | −0.004602 |
| SULT1B1 | −0.004603 |
| TMEM155 | −0.004604 |
| ARHGAP9 | −0.004609 |
| SOD2 | −0.004609 |
| SLC16A6 | −0.004619 |
| SYPL1 | −0.004620 |
| RGS1 | −0.004623 |
| ZNFX1 | −0.004626 |
| SCARF2 | −0.004626 |
| PPP1R3C | −0.004627 |
| WDR81 | −0.004629 |
| INPP5K | −0.004630 |
| CXCL10 | −0.004630 |
| SMARCA2 | −0.004630 |
| HSD17B8 | −0.004631 |
| CDKN2B | −0.004631 |
| MCOLN2 | −0.004631 |
| CD58 | −0.004636 |
| MRPL14 | −0.004636 |
| NLRP2 | −0.004638 |
| EBI3 | −0.004641 |
| A4GALT | −0.004641 |
| HPS1 | −0.004646 |
| GLTP | −0.004648 |
| NRG3 | −0.004652 |
| TWF2 | −0.004653 |
| ABCA9 | −0.004654 |
| MTM1 | −0.004654 |
| HSD3B7 | −0.004655 |
| WNT9B | −0.004655 |
| SNX18 | −0.004659 |
| PTPRU | −0.004660 |
| TLR10 | −0.004660 |
| PRSS33 | −0.004660 |
| ST6GAL1 | −0.004662 |
| VPS18 | −0.004662 |
| RNPEPL1 | −0.004662 |
| C4orf22 | −0.004662 |
| LRCOL1 | −0.004666 |
| PPP1R16B | −0.004667 |
| DACT3 | −0.004668 |
| CROCC2 | −0.004670 |
| ANK3 | −0.004670 |
| C10orf105 | −0.004676 |
| IQGAP1 | −0.004683 |
| APOC4 | −0.004685 |
| NAMPT | −0.004688 |
| LAMP1 | −0.004689 |
| TMCC3 | −0.004691 |
| FUT7 | −0.004691 |
| CAPNS1 | −0.004691 |
| SAA2 | −0.004692 |
| LACTB | −0.004693 |
| ANXA6 | −0.004694 |
| ARRDC2 | −0.004694 |
| FRK | −0.004695 |
| SLC12A8 | −0.004702 |
| RECK | −0.004703 |
| NME8 | −0.004704 |
| MICALL1 | −0.004704 |
| WFDC13 | −0.004706 |
| RNASEL | −0.004708 |
| ERGIC1 | −0.004708 |
| TEAD4 | −0.004710 |
| CASP7 | −0.004711 |
| KLK11 | −0.004714 |
| KCNE3 | −0.004714 |
| AMHR2 | −0.004715 |
| BCL6 | −0.004717 |
| PSMB8 | −0.004718 |
| PLAGL1 | −0.004720 |
| CLTB | −0.004721 |
| DHH | −0.004721 |
| FAM167B | −0.004721 |
| SLC9C2 | −0.004722 |
| RHBDF1 | −0.004723 |
| LTBR | −0.004724 |
| CNDP2 | −0.004731 |
| PNMAL2 | −0.004734 |
| ZNF835 | −0.004736 |
| GHDC | −0.004736 |
| ARSJ | −0.004746 |
| HSBP1L1 | −0.004747 |
| SDCBP | −0.004747 |
| SOCS1 | −0.004749 |
| KIRREL | −0.004750 |
| TMEM87A | −0.004750 |
| ARHGAP27 | −0.004751 |
| RAI14 | −0.004752 |
| NPC1 | −0.004753 |
| GPR182 | −0.004755 |
| TPRG1L | −0.004758 |
| PTGDR | −0.004760 |
| FERMT2 | −0.004761 |
| EPS8 | −0.004762 |
| PTPN6 | −0.004764 |
| ZCWPW2 | −0.004764 |
| CD300A | −0.004770 |
| MYH13 | −0.004771 |
| TMEM98 | −0.004771 |
| CACHD1 | −0.004773 |
| GBA3 | −0.004776 |
| DKKL1 | −0.004780 |
| 1PH4 | −0.004785 |
| RPS6KA1 | −0.004785 |
| ARPC4 | −0.004787 |
| WWP2 | −0.004789 |
| IL27 | −0.004790 |
| DOCK9 | −0.004796 |
| GSTO2 | −0.004798 |
| TTC16 | −0.004800 |
| FAM150A | −0.004807 |
| CD79A | −0.004808 |
| DDO | −0.004809 |
| CPEB2 | −0.004812 |
| PLEKHB2 | −0.004817 |
| RSPH10B2 | −0.004817 |
| GAA | −0.004821 |
| TICAM2 | −0.004822 |
| SGCD | −0.004822 |
| SLA2 | −0.004824 |
| BRI3 | −0.004826 |
| NFIC | −0.004827 |
| TMEM219 | −0.004828 |
| TKTL1 | −0.004829 |
| PTPRG | −0.004829 |
| CAPN1 | −0.004831 |
| PK3R6 | −0.004833 |
| MAN2B2 | −0.004836 |
| ALAS1 | −0.004843 |
| OPHN1 | −0.004846 |
| SLC39A11 | −0.004847 |
| SMPD1 | −0.004851 |
| TMBIM6 | −0.004852 |
| CCPG1 | −0.004852 |
| HELT | −0.004853 |
| SPATS2L | −0.004855 |
| HECW2 | −0.004861 |
| GLT8D2 | −0.004862 |
| AVPR2 | −0.004869 |
| CFAP58 | −0.004872 |
| NR1H4 | −0.004879 |
| PTPRO | −0.004882 |
| ASB2 | −0.004886 |
| UBE2E2 | −0.004887 |
| COL6A1 | −0.004888 |
| MED11 | −0.004890 |
| PTX3 | −0.004895 |
| TMC7 | −0.004901 |
| GEM | −0.004902 |
| MBNL1 | −0.004905 |
| TMEM140 | −0.004906 |
| DOK3 | −0.004908 |
| OCLN | −0.004910 |
| LTBP1 | −0.004910 |
| RGS10 | −0.004918 |
| SLC8B1 | −0.004920 |
| PXN | −0.004922 |
| ZFP42 | −0.004924 |
| PAX8 | −0.004925 |
| TMEM156 | −0.004931 |
| ARPIN | −0.004935 |
| HERC6 | −0.004935 |
| IRF4 | −0.004935 |
| RUNX3 | −0.004939 |
| ENAM | −0.004939 |
| PPFEBP1 | −0.004941 |
| AP5B1 | −0.004942 |
| ARHGEF26 | −0.004944 |
| ARL14EPL | −0.004945 |
| NFKB2 | −0.004946 |
| PHACTR1 | −0.004948 |
| NPY1R | −0.004949 |
| HMGN5 | −0.004949 |
| BMP6 | −0.004949 |
| MEIKIN | −0.004951 |
| CPNE2 | −0.004953 |
| ARL14 | −0.004954 |
| NDST1 | −0.004957 |
| PMM1 | −0.004958 |
| SIX5 | −0.004958 |
| TMPRSS4 | −0.004958 |
| TMEM120A | −0.004959 |
| DCUN1D3 | −0.004959 |
| MFGE8 | −0.004960 |
| PPP1R3B | −0.004962 |
| LHFPL2 | −0.004963 |
| SNAP23 | −0.004963 |
| ALDH3B2 | −0.004968 |
| HOXD1 | −0.004970 |
| ATP6V0D1 | −0.004973 |
| ACSS1 | −0.004974 |
| KRT6A | −0.004979 |
| TEX264 | −0.004984 |
| DUSP7 | −0.004985 |
| AZGP1 | −0.004990 |
| GALNT10 | −0.004991 |
| ID3 | −0.004992 |
| ZBTB47 | −0.004996 |
| ITIH3 | −0.005000 |
| NACC2 | −0.005000 |
| TIGIT | −0.005002 |
| OCA2 | −0.005003 |
| IRF1 | −0.005004 |
| TMEM63B | −0.005005 |
| GBP5 | −0.005006 |
| GRASP | −0.005009 |
| CRY2 | −0.005009 |
| ECM1 | −0.005010 |
| ITGAD | −0.005010 |
| MROH6 | −0.005011 |
| AGA | −0.005013 |
| LEPROT | −0.005014 |
| TBXA2R | −0.005017 |
| FURIN | −0.005017 |
| GALC | −0.005018 |
| FAM110C | −0.005018 |
| SH2D2A | −0.005018 |
| HRC | −0.005019 |
| EIF5AL1 | −0.005023 |
| SLC10A3 | −0.005023 |
| WIPF1 | −0.005025 |
| KLHDC7B | −0.005025 |
| ABHD17C | −0.005028 |
| SERPINB8 | −0.005032 |
| PLD1 | −0.005033 |
| GGT5 | −0.005041 |
| GPRC5C | −0.005044 |
| NBPF14 | −0.005050 |
| SVOPL | −0.005051 |
| STS | −0.005055 |
| GTSF1 | −0.005056 |
| ITPKB | −0.005059 |
| RANBP3L | −0.005063 |
| LPA | −0.005064 |
| HTRA3 | −0.005069 |
| PEAK1 | −0.005071 |
| CAB39L | −0.005073 |
| CLEC4C | −0.005073 |
| CDKL5 | −0.005075 |
| TTPA | −0.005079 |
| NPL | −0.005081 |
| ADAM29 | −0.005082 |
| DSTN | −0.005083 |
| FAM129A | −0.005084 |
| VCAMI | −0.005085 |
| SNX25 | −0.005088 |
| RCAN2 | −0.005088 |
| IFFO2 | −0.005089 |
| FAM198B | −0.005093 |
| RHOJ | −0.005093 |
| CD177 | −0.005101 |
| LRCH2 | −0.005105 |
| NTRK3 | −0.005106 |
| ADAM9 | −0.005108 |
| TRAPPC3L | −0.005109 |
| SHISA4 | −0.005109 |
| CCL4L2 | −0.005113 |
| SLC43A3 | −0.005115 |
| SIGLEC8 | −0.005117 |
| CASKIN2 | −0.005119 |
| CFAP47 | −0.005121 |
| COL4A5 | −0.005121 |
| LAPTM4A | −0.005122 |
| ALDH3A2 | −0.005125 |
| JOSD2 | −0.005126 |
| OSBPL5 | −0.005128 |
| STEAP2 | −0.005131 |
| POR | −0.005133 |
| PARP4 | −0.005135 |
| SECISBP2L | −0.005138 |
| MAP3K15 | −0.005139 |
| LY6G6F | −0.005139 |
| MANBA | −0.005139 |
| BTN3A2 | −0.005145 |
| GUCY2D | −0.005145 |
| KLHL38 | −0.005148 |
| PCSK6 | −0.005148 |
| PHLDB2 | −0.005148 |
| GALNT15 | −0.005149 |
| FAM65A | −0.005153 |
| MXRA7 | −0.005153 |
| ST6GALNAC5 | −0.005155 |
| RAET1G | −0.005157 |
| TNIP1 | −0.005160 |
| IRX5 | −0.005164 |
| MAP3K6 | −0.005165 |
| ISM1 | −0.005165 |
| CLEC12B | −0.005166 |
| ERAP1 | −0.005168 |
| PDE4B | −0.005169 |
| ABCA1 | −0.005169 |
| ACOT1 | −0.005171 |
| CABLES1 | −0.005171 |
| MYO5C | −0.005173 |
| UST | −0.005174 |
| ST3GAL6 | −0.005174 |
| TMEM54 | −0.005174 |
| DAB2IP | −0.005176 |
| SLC39A1 | −0.005176 |
| MFSD5 | −0.005184 |
| MAN2B1 | −0.005185 |
| CD1D | −0.005193 |
| KRT18 | −0.005194 |
| SLC6A20 | −0.005195 |
| APCDD1 | −0.005197 |
| NDN | −0.005203 |
| SLC6A6 | −0.005204 |
| DOK1 | −0.005206 |
| IFI6 | −0.005211 |
| MS4A6E | −0.005212 |
| C9orf24 | −0.005215 |
| BDH2 | −0.005217 |
| VWC2 | −0.005228 |
| N4BP1 | −0.005231 |
| RAB40A | −0.005238 |
| PIK3C2G | −0.005245 |
| MAGI3 | −0.005245 |
| FAM198A | −0.005245 |
| DOCK11 | −0.005248 |
| RSAD2 | −0.005249 |
| SBDS | −0.005249 |
| LRRC6 | −0.005250 |
| DPYS | −0.005250 |
| KIFC3 | −0.005250 |
| ELF1 | −0.005255 |
| PLIN2 | −0.005257 |
| PLK2 | −0.005259 |
| SRPX2 | −0.005259 |
| RALB | −0.005261 |
| PLEKHN1 | −0.005264 |
| RAB3IL1 | −0.005267 |
| CCDC65 | −0.005267 |
| TPM4 | −0.005271 |
| EPB41L4A | −0.005272 |
| TIFAB | −0.005277 |
| LMF1 | −0.005279 |
| TRAPPCI | −0.005284 |
| RRBP1 | −0.005286 |
| TCF7L2 | −0.005287 |
| UTS2 | −0.005290 |
| PRSS23 | −0.005291 |
| MXRA8 | −0.005294 |
| RASSF5 | −0.005295 |
| SSH3 | −0.005299 |
| H2AFJ | −0.005301 |
| RHOA | −0.005302 |
| TSLP | −0.005313 |
| NR1H2 | −0.005314 |
| SYNPO2L | −0.005314 |
| SERTAD3 | −0.005315 |
| MT1X | −0.005316 |
| ZNF521 | −0.005319 |
| NUDT18 | −0.005319 |
| ZDHHC7 | −0.005319 |
| ADAP2 | −0.005327 |
| EFHC2 | −0.005327 |
| TNFRSF9 | −0.005330 |
| EDA2R | −0.005333 |
| HSD17B11 | −0.005337 |
| PACRG | −0.005339 |
| REEPS | −0.005340 |
| GFRA1 | −0.005343 |
| SPRYD7 | −0.005344 |
| FAM26E | −0.005344 |
| PDE2A | −0.005349 |
| PKDCC | −0.005351 |
| VIT | −0.005351 |
| HEXB | −0.005352 |
| MMP14 | −0.005353 |
| SPRY1 | −0.005363 |
| NR4A2 | −0.005365 |
| C17orf64 | −0.005369 |
| DCDC2 | −0.005371 |
| WWC3 | −0.005375 |
| HEXA | −0.005376 |
| TMSB4X | −0.005379 |
| TAPBP | −0.005379 |
| SNX30 | −0.005380 |
| ELOVL1 | −0.005385 |
| ADGRG3 | −0.005386 |
| STAT5A | −0.005391 |
| FEZ2 | −0.005392 |
| DHDH | −0.005393 |
| SERINC5 | −0.005394 |
| IFIT5 | −0.005395 |
| ADRB3 | −0.005399 |
| MSRA | −0.005403 |
| BIRC7 | −0.005404 |
| ADAMTS3 | −0.005405 |
| STAT4 | −0.005406 |
| ULBP3 | −0.005407 |
| FRRS1 | −0.005407 |
| DPYSL2 | −0.005408 |
| DNAI2 | −0.005408 |
| FZD5 | −0.005409 |
| DHRS7B | −0.005410 |
| COL6A2 | −0.005411 |
| ABCC9 | −0.005412 |
| C11orf45 | −0.005414 |
| COL17A1 | −0.005414 |
| MAP1LC3B2 | −0.005414 |
| APOBEC3G | −0.005415 |
| USP25 | −0.005416 |
| PRR15 | −0.005418 |
| TMEM43 | −0.005420 |
| LTK | −0.005422 |
| APOD | −0.005425 |
| SLC5A5 | −0.005426 |
| PSTPIP1 | −0.005426 |
| PYGL | −0.005428 |
| ZFP57 | −0.005434 |
| GXYLT2 | −0.005435 |
| TREML2 | −0.005437 |
| LRRK1 | −0.005439 |
| CHST3 | −0.005440 |
| EFNA4 | −0.005441 |
| CTSA | −0.005442 |
| CLN5 | −0.005444 |
| DOC2B | −0.005445 |
| MAFF | −0.005445 |
| CDS1 | −0.005446 |
| ACSS3 | −0.005448 |
| SGMS1 | −0.005449 |
| S100A13 | −0.005450 |
| GJC2 | −0.005453 |
| CCDC42 | −0.005456 |
| CDHR3 | −0.005458 |
| KIR2DL4 | −0.005459 |
| CC2D2A | −0.005461 |
| CMTM3 | −0.005463 |
| RSU1 | −0.005464 |
| KANK1 | −0.005465 |
| HTRA1 | −0.005468 |
| SAMD5 | −0.005469 |
| TRAM1 | −0.005469 |
| KLHL5 | −0.005469 |
| TC2N | −0.005476 |
| P2RY8 | −0.005481 |
| LAMA4 | −0.005488 |
| RAP2B | −0.005488 |
| HDAC11 | −0.005492 |
| PHOSPHO1 | −0.005495 |
| PLK3 | −0.005499 |
| B3GALT4 | −0.005503 |
| TCEA3 | −0.005504 |
| UXS1 | −0.005504 |
| EDEM2 | −0.005505 |
| CCL3 | −0.005508 |
| KLHDC8B | −0.005513 |
| VTCN1 | −0.005513 |
| MGMT | −0.005514 |
| PROK2 | −0.005515 |
| HSD17B2 | −0.005515 |
| CTDSP1 | −0.005516 |
| ZBTB42 | −0.005516 |
| NFKB1 | −0.005521 |
| AFAP1L1 | −0.005522 |
| GULP1 | −0.005523 |
| FHOD1 | −0.005524 |
| ACSL1 | −0.005526 |
| ANTXR2 | −0.005529 |
| C12orf74 | −0.005529 |
| MAP4 | −0.005530 |
| TACC2 | −0.005530 |
| COLGALT2 | −0.005530 |
| SMC03 | −0.005531 |
| FCRLB | −0.005535 |
| PLEC | −0.005536 |
| OIT3 | −0.005537 |
| PARP15 | −0.005538 |
| RAP1A | −0.005541 |
| P2RY6 | −0.005541 |
| LILRA4 | −0.005543 |
| WDR78 | −0.005544 |
| IL31RA | −0.005546 |
| PLCG2 | −0.005549 |
| NEK5 | −0.005550 |
| FCRL3 | −0.005550 |
| PARVB | −0.005555 |
| ALDH1A3 | −0.005555 |
| MYH9 | −0.005557 |
| C5 | −0.005561 |
| MUC3A | −0.005564 |
| C22orf15 | −0.005565 |
| UTRN | −0.005567 |
| FAM65B | −0.005568 |
| ARHGEF3 | −0.005568 |
| MEI1 | −0.005568 |
| SLC7A2 | −0.005569 |
| TNC | −0.005575 |
| MCC | −0.005576 |
| DHX58 | −0.005577 |
| GAB1 | −0.005580 |
| VASP | −0.005583 |
| SH2D4B | −0.005584 |
| MAL2 | −0.005590 |
| CCDC96 | −0.005591 |
| CBX7 | −0.005593 |
| ACPP | −0.005594 |
| TLN1 | −0.005594 |
| STK10 | −0.005598 |
| LXN | −0.005601 |
| TMC4 | −0.005601 |
| MRAS | −0.005604 |
| TNKS1BP1 | −0.005605 |
| GSAP | −0.005605 |
| LRMP | −0.005606 |
| GPR68 | −0.005606 |
| EYA4 | −0.005609 |
| ARHGAP25 | −0.005609 |
| TMEM220 | −0.005612 |
| LIMD1 | −0.005615 |
| VSTM2L | −0.005616 |
| HAAO | −0.005617 |
| ABHD6 | −0.005626 |
| VAT1 | −0.005630 |
| APOL6 | −0.005631 |
| NINJ1 | −0.005633 |
| ETS1 | −0.005633 |
| BTD | −0.005634 |
| TOM1 | −0.005635 |
| C12orf49 | −0.005637 |
| JMJD7 | −0.005639 |
| PAG1 | −0.005640 |
| C19orf84 | −0.005641 |
| ATP1A1 | −0.005648 |
| CAMK1G | −0.005650 |
| CCDC113 | −0.005650 |
| ASL | −0.005651 |
| PNKD | −0.005653 |
| CARD10 | −0.005656 |
| GLOD5 | −0.005659 |
| F2RL1 | −0.005659 |
| CD209 | −0.005660 |
| LRRC46 | −0.005661 |
| ISG20 | −0.005661 |
| EOGT | −0.005661 |
| RASGRP1 | −0.005662 |
| FAM107B | −0.005663 |
| ST5 | −0.005665 |
| ARL4D | −0.005668 |
| EFNB1 | −0.005669 |
| NUGGC | −0.005669 |
| C1QTNF1 | −0.005672 |
| CSRP1 | −0.005675 |
| AQP2 | −0.005682 |
| DENND3 | −0.005689 |
| SLAMF7 | −0.005690 |
| SUCNR1 | −0.005694 |
| TIPARP | −0.005696 |
| CXCR5 | −0.005698 |
| LDHB | −0.005702 |
| DEFB4A | −0.005704 |
| SIDT1 | −0.005704 |
| ACTB | −0.005704 |
| LENG9 | −0.005706 |
| TRIP10 | −0.005707 |
| ARHGAP29 | −0.005710 |
| NKX2-1 | −0.005711 |
| NAP1L2 | −0.005711 |
| CEACAM1 | −0.005712 |
| LGALSL | −0.005719 |
| MUC5AC | −0.005719 |
| ELL2 | −0.005725 |
| ARID5B | −0.005728 |
| KRT5 | −0.005728 |
| IL10 | −0.005732 |
| RNF39 | −0.005734 |
| PREXI | −0.005741 |
| RNLS | −0.005742 |
| SYPL2 | −0.005745 |
| CFAP74 | −0.005745 |
| RGS9 | −0.005746 |
| WBP1L | −0.005749 |
| PRRX2 | −0.005751 |
| FES | −0.005753 |
| PK3AP1 | −0.005755 |
| SLC16A2 | −0.005755 |
| SLC35C1 | −0.005763 |
| GLI1 | −0.005764 |
| TEAD3 | −0.005765 |
| ZC4H2 | −0.005766 |
| VAMP3 | −0.005766 |
| THEM5 | −0.005767 |
| CACNA2D4 | −0.005768 |
| SMIM3 | −0.005768 |
| SMIM10L2B | −0.005771 |
| PTPRR | −0.005778 |
| SLC2A10 | −0.005784 |
| GALE | −0.005785 |
| PPARD | −0.005786 |
| ETFB | −0.005788 |
| ANKRD22 | −0.005789 |
| WNT10A | −0.005790 |
| TMEM2 | −0.005792 |
| MRVI1 | −0.005793 |
| SFXN3 | −0.005794 |
| SPEF2 | −0.005798 |
| C1orf194 | −0.005799 |
| MYO1B | −0.005800 |
| FZD4 | −0.005800 |
| LURAP1L | −0.005802 |
| STAB2 | −0.005803 |
| CLECL1 | −0.005804 |
| GLIPR1 | −0.005805 |
| BLNK | −0.005805 |
| KLRC1 | −0.005806 |
| LAP3 | −0.005810 |
| SCAMP2 | −0.005812 |
| ACY1 | −0.005816 |
| TNNT2 | −0.005822 |
| NR3C1 | −0.005825 |
| RELB | −0.005827 |
| ATF7IP2 | −0.005828 |
| PHEX | −0.005829 |
| TJP1 | −0.005829 |
| PARK2 | −0.005837 |
| POLD4 | −0.005838 |
| ABLIM1 | −0.005842 |
| KLK8 | −0.005843 |
| ATF3 | −0.005844 |
| GDF15 | −0.005847 |
| MDFI | −0.005852 |
| GABRE | −0.005856 |
| HS6ST2 | −0.005856 |
| TICAM1 | −0.005860 |
| C1QTNF2 | −0.005870 |
| RHOH | −0.005873 |
| PCOLCE | −0.005877 |
| CHST15 | −0.005880 |
| TBC1D10C | −0.005881 |
| TRIM8 | −0.005882 |
| TRADD | −0.005887 |
| CTAGE4 | −0.005892 |
| NFKBIZ | −0.005896 |
| GPR141 | −0.005904 |
| EFNB2 | −0.005907 |
| WARS | −0.005907 |
| ANXA5 | −0.005908 |
| PTPN7 | −0.005908 |
| DUSP3 | −0.005910 |
| SPSB1 | −0.005911 |
| PFKFB3 | −0.005914 |
| SAMD12 | −0.005914 |
| TAPBPL | −0.005914 |
| PALD1 | −0.005915 |
| MOB3C | −0.005917 |
| SLC9A1 | −0.005918 |
| KLRK1 | −0.005920 |
| PLEKHA4 | −0.005923 |
| RNF43 | −0.005925 |
| PEG3 | −0.005929 |
| AN03 | −0.005929 |
| LHFPL3 | −0.005930 |
| CD248 | −0.005930 |
| PLCH1 | −0.005941 |
| TYMP | −0.005942 |
| DNAH6 | −0.005943 |
| TPT1 | −0.005943 |
| GPBAR1 | −0.005949 |
| TXNDC11 | −0.005949 |
| AR | −0.005950 |
| EMB | −0.005951 |
| TMEM40 | −0.005951 |
| SLC44A3 | −0.005953 |
| GPR17 | −0.005954 |
| SASH1 | −0.005955 |
| PLA2R1 | −0.005955 |
| ARHGAP20 | −0.005957 |
| IRF5 | −0.005958 |
| PSTPIP2 | −0.005964 |
| SLC22A18 | −0.005968 |
| SEMA7A | −0.005972 |
| SEMA3A | −0.005973 |
| TAOK3 | −0.005973 |
| MAP2K3 | −0.005975 |
| SLC24A3 | −0.005975 |
| MT2A | −0.005978 |
| FABP6 | −0.005979 |
| ADGRL4 | −0.005979 |
| PRDX5 | −0.005983 |
| SWAP70 | −0.005988 |
| ERRFI1 | −0.005992 |
| ORAI3 | −0.005994 |
| ADH6 | −0.005995 |
| GRAP | −0.005996 |
| OLFML2A | −0.005998 |
| MACROD2 | −0.005999 |
| MX1 | −0.005999 |
| GAL3ST1 | −0.006001 |
| BNC1 | −0.006001 |
| RBPMS2 | −0.006005 |
| MST1R | −0.006006 |
| DENND1C | −0.006008 |
| SQSTM1 | −0.006009 |
| RPH3AL | −0.006012 |
| FTH1 | −0.006015 |
| LAYN | −0.006016 |
| CXCL11 | −0.006018 |
| AZU1 | −0.006019 |
| MYOZ3 | −0.006020 |
| RGS2 | −0.006020 |
| MGAT1 | −0.006022 |
| XKRX | −0.006024 |
| ADGRL2 | −0.006027 |
| SLC16A3 | −0.006030 |
| NECAB1 | −0.006032 |
| CCDC8 | −0.006037 |
| OR6K3 | −0.006039 |
| CACNA1S | −0.006042 |
| RUNX1 | −0.006043 |
| RGS19 | −0.006044 |
| SGPP2 | −0.006045 |
| COL12A1 | −0.006047 |
| ITGA9 | −0.006049 |
| FMNL1 | −0.006051 |
| CSGALNACT1 | −0.006052 |
| ORAI1 | −0.006052 |
| GPR4 | −0.006053 |
| EXOC3L4 | −0.006059 |
| GSTO1 | −0.006062 |
| LAT2 | −0.006068 |
| NQO1 | −0.006068 |
| PEAR1 | −0.006072 |
| ATP6V0E1 | −0.006077 |
| NALCN | −0.006077 |
| GUCA2B | −0.006083 |
| IL13RA1 | −0.006085 |
| KLRG2 | −0.006086 |
| SH2D3A | −0.006088 |
| PGM1 | −0.006089 |
| LTC4S | −0.006093 |
| GRIA1 | −0.006094 |
| RBMS1 | −0.006096 |
| ADCYAP1R1 | −0.006099 |
| ZCCHC5 | −0.006108 |
| SIDT2 | −0.006120 |
| SOX18 | −0.006120 |
| PLD3 | −0.006121 |
| UGT2B7 | −0.006125 |
| F8 | −0.006126 |
| CCL8 | −0.006127 |
| CRYL1 | −0.006132 |
| PTPRM | −0.006132 |
| KCNJ16 | −0.006133 |
| ATXN1 | −0.006136 |
| ASB12 | −0.006140 |
| TGFB1I1 | −0.006146 |
| UBA7 | −0.006147 |
| ECM2 | −0.006148 |
| MTMR8 | −0.006148 |
| PRR13 | −0.006149 |
| VCL | −0.006150 |
| LMTK3 | −0.006151 |
| SPTBN1 | −0.006151 |
| CYB5R2 | −0.006153 |
| ARHGAP15 | −0.006159 |
| GALNT12 | −0.006161 |
| FCMR | −0.006161 |
| 43526 | −0.006162 |
| HSPG2 | −0.006163 |
| CD99 | −0.006166 |
| NPNT | −0.006169 |
| TNFSF15 | −0.006182 |
| GLB1 | −0.006183 |
| C1orf210 | −0.006185 |
| KCNAB1 | −0.006188 |
| KERA | −0.006196 |
| CHSY3 | −0.006200 |
| TMIGD2 | −0.006206 |
| DIAPH2 | −0.006209 |
| PLAC4 | −0.006210 |
| PRDM1 | −0.006210 |
| DMRTC1B | −0.006215 |
| KLK13 | −0.006216 |
| BTN3A3 | −0.006217 |
| ANTXR1 | −0.006223 |
| CD70 | −0.006229 |
| RNF145 | −0.006229 |
| CYP3A5 | −0.006237 |
| NRP1 | −0.006240 |
| PRKCH | −0.006245 |
| MMEL1 | −0.006248 |
| REEP6 | −0.006249 |
| SH3KBP1 | −0.006253 |
| RNF135 | −0.006259 |
| LGALS4 | −0.006260 |
| PPP1R1B | −0.006263 |
| PLCB2 | −0.006266 |
| IKZF3 | −0.006268 |
| PRSS57 | −0.006269 |
| OAS1 | −0.006272 |
| SPRYD3 | −0.006273 |
| SORBS3 | −0.006274 |
| 1QSEC2 | −0.006274 |
| CEBPB | −0.006276 |
| BAIAP2 | −0.006277 |
| CD180 | −0.006282 |
| METTL7A | −0.006284 |
| PARP3 | −0.006284 |
| TUBB1 | −0.006285 |
| IL15 | −0.006289 |
| SP110 | −0.006289 |
| LPCAT3 | −0.006294 |
| NRK | −0.006296 |
| MYO7B | −0.006296 |
| CD79B | −0.006297 |
| UBASH3B | −0.006300 |
| RASAL3 | −0.006302 |
| FASLG | −0.006305 |
| ENDOU | −0.006305 |
| GRIK4 | −0.006308 |
| SMAD7 | −0.006310 |
| SLC22A18AS | −0.006314 |
| CYB5R3 | −0.006319 |
| FLI1 | −0.006321 |
| TRIM21 | −0.006323 |
| FLT4 | −0.006324 |
| SHISA5 | −0.006324 |
| EPSTI1 | −0.006325 |
| REST | −0.006332 |
| NTN1 | −0.006334 |
| IFITM10 | −0.006335 |
| ARID5A | −0.006337 |
| DCANP1 | −0.006340 |
| PSMB9 | −0.006341 |
| OPN3 | −0.006341 |
| OSTF1 | −0.006342 |
| SLC5A7 | −0.006342 |
| CFAP45 | −0.006343 |
| EPGN | −0.006349 |
| LY6D | −0.006350 |
| SIRPG | −0.006351 |
| ETHE1 | −0.006354 |
| GLIS2 | −0.006358 |
| UBASH3A | −0.006361 |
| IL18RAP | −0.006364 |
| MXRA5 | −0.006364 |
| ZNF474 | −0.006365 |
| TUBB6 | −0.006369 |
| KIR2DS4 | −0.006369 |
| AIM1 | −0.006371 |
| RBKS | −0.006372 |
| PXMP4 | −0.006375 |
| MYBPHL | −0.006378 |
| SMAD3 | −0.006382 |
| TM4SF19 | −0.006383 |
| HES2 | −0.006384 |
| RNH1 | −0.006385 |
| ACSL4 | −0.006387 |
| BCAR3 | −0.006387 |
| C1orf198 | −0.006388 |
| RAET1E | −0.006389 |
| LITAF | −0.006400 |
| IRAK2 | −0.006401 |
| UBD | −0.006404 |
| MSMO1 | −0.006408 |
| MFAP3L | −0.006409 |
| SYT3 | −0.006410 |
| TMEM133 | −0.006410 |
| AGTRAP | −0.006413 |
| GPC5 | −0.006419 |
| HLA-A | −0.006421 |
| NAB2 | −0.006422 |
| SLC43A2 | −0.006424 |
| LRRC43 | −0.006424 |
| APOE | −0.006425 |
| LAX1 | −0.006430 |
| CRB3 | −0.006433 |
| TMEM64 | −0.006436 |
| C4orf32 | −0.006439 |
| STK17B | −0.006441 |
| CYP4Z1 | −0.006446 |
| TCTE1 | −0.006446 |
| CHRM2 | −0.006446 |
| C1QTNF5 | −0.006447 |
| FAM167A | −0.006449 |
| DNAAF1 | −0.006449 |
| B3GNT6 | −0.006451 |
| RNF212 | −0.006455 |
| RIN1 | −0.006460 |
| PDCD1 | −0.006461 |
| CMTM8 | −0.006465 |
| HGD | −0.006466 |
| H6PD | −0.006469 |
| CMTM2 | −0.006469 |
| RMDN3 | −0.006473 |
| ID1 | −0.006476 |
| IGFALS | −0.006477 |
| KIR2DL1 | −0.006478 |
| P2RX7 | −0.006481 |
| ACKR2 | −0.006481 |
| EXD1 | −0.006483 |
| IFT57 | −0.006484 |
| EZR | −0.006487 |
| NXN | −0.006491 |
| TTC7A | −0.006492 |
| ASAP3 | −0.006493 |
| LDLRAD2 | −0.006494 |
| HS3ST2 | −0.006497 |
| CNTN1 | −0.006501 |
| OTUD1 | −0.006501 |
| SULT1A1 | −0.006502 |
| PPP2R5A | −0.006514 |
| CFAP99 | −0.006515 |
| RYR1 | −0.006517 |
| KANK2 | −0.006518 |
| IFI44L | −0.006521 |
| SYDE1 | −0.006530 |
| MMP15 | −0.006531 |
| PRRG4 | −0.006533 |
| JADE2 | −0.006534 |
| CASP4 | −0.006534 |
| ZDHHC3 | −0.006537 |
| KIF17 | −0.006538 |
| NCOA7 | −0.006541 |
| PPM1M | −0.006543 |
| GJA4 | −0.006548 |
| TNFRSF13B | −0.006552 |
| ZNF385D | −0.006554 |
| PTGIR | −0.006555 |
| C1RL | −0.006556 |
| PBXIP1 | −0.006556 |
| TTC22 | −0.006556 |
| PCDH7 | −0.006556 |
| VSIG10L | −0.006560 |
| BGN | −0.006566 |
| AKAP13 | −0.006568 |
| LYL1 | −0.006569 |
| FBXO6 | −0.006572 |
| KCNE1 | −0.006579 |
| FOXO1 | −0.006584 |
| ADCY4 | −0.006593 |
| ATP2B4 | −0.006596 |
| TLR1 | −0.006599 |
| MAN1A1 | −0.006600 |
| ADGRE2 | −0.006600 |
| CTDSPL | −0.006601 |
| SAA1 | −0.006609 |
| SOWAHD | −0.006614 |
| EHD4 | −0.006617 |
| SRXN1 | −0.006633 |
| KIAA0513 | −0.006634 |
| MEOX1 | −0.006635 |
| ADCY9 | −0.006636 |
| TPST2 | −0.006636 |
| SUCLG2 | −0.006645 |
| MS4A14 | −0.006648 |
| MS4A6A | −0.006657 |
| FGF2 | −0.006659 |
| SLC37A2 | −0.006660 |
| EVA1C | −0.006661 |
| CXCL8 | −0.006661 |
| IRAK3 | −0.006664 |
| ARHGAP24 | −0.006668 |
| TSPAN7 | −0.006668 |
| DDX43 | −0.006671 |
| NUCB1 | −0.006672 |
| CHP2 | −0.006677 |
| CHRNA2 | −0.006677 |
| LGALS9B | −0.006677 |
| HPCAL1 | −0.006677 |
| CCNJL | −0.006681 |
| MSMB | −0.006684 |
| MEOX2 | −0.006687 |
| TMEM102 | −0.006693 |
| S100A2 | −0.006693 |
| RORC | −0.006696 |
| NOTUM | −0.006696 |
| FAM212A | −0.006698 |
| ITIH2 | −0.006699 |
| IQGAP2 | −0.006702 |
| PCTP | −0.006703 |
| SOST | −0.006711 |
| ITPRIPL2 | −0.006712 |
| CDC42EP2 | −0.006714 |
| SLC6A9 | −0.006719 |
| C6orf118 | −0.006720 |
| GAS2L1 | −0.006721 |
| PSAP | −0.006723 |
| LAIR2 | −0.006725 |
| UNC5C | −0.006726 |
| KIF13A | −0.006728 |
| DRC1 | −0.006729 |
| NOS3 | −0.006735 |
| ABHD14B | −0.006735 |
| AMN | −0.006737 |
| FAM49A | −0.006743 |
| CYP4X1 | −0.006743 |
| ENPP6 | −0.006762 |
| RHOC | −0.006764 |
| RIMBP3C | −0.006767 |
| ESR1 | −0.006774 |
| MEF2C | −0.006778 |
| M1AP | −0.006778 |
| WFDC5 | −0.006781 |
| PNPLA4 | −0.006782 |
| WFIKKN2 | −0.006784 |
| PDLIM4 | −0.006788 |
| ACTA2 | −0.006790 |
| RAB34 | −0.006791 |
| AMBP | −0.006795 |
| DLX4 | −0.006796 |
| C11orf70 | −0.006798 |
| CCL4 | −0.006801 |
| SIGLEC12 | −0.006804 |
| LDLRAP1 | −0.006805 |
| COL8A2 | −0.006806 |
| 43526 | −0.006810 |
| TNS2 | −0.006814 |
| CAPN2 | −0.006820 |
| SLC44A2 | −0.006823 |
| PDE4C | −0.006830 |
| CMTM5 | −0.006830 |
| ADORA3 | −0.006831 |
| TDRD9 | −0.006832 |
| PLA2G15 | −0.006837 |
| CD27 | −0.006841 |
| DPP6 | −0.006848 |
| FOXA2 | −0.006852 |
| TMEM109 | −0.006853 |
| FGD2 | −0.006858 |
| GNG12 | −0.006858 |
| IFI16 | −0.006860 |
| CXCR6 | −0.006860 |
| IL6ST | −0.006861 |
| LMNTD1 | −0.006862 |
| LANCL3 | −0.006871 |
| C22orf23 | −0.006872 |
| STARD13 | −0.006875 |
| FMO1 | −0.006876 |
| HEBP1 | −0.006885 |
| PARD6B | −0.006891 |
| TMEM88 | −0.006893 |
| PSMB10 | −0.006895 |
| ZEB2 | −0.006895 |
| PAK6 | −0.006897 |
| ST6GALNAC4 | −0.006899 |
| PLAT | −0.006902 |
| CYP4F12 | −0.006909 |
| RAB11FIP5 | −0.006916 |
| ACSM1 | −0.006918 |
| GLP2R | −0.006918 |
| PLAU | −0.006918 |
| GABARAPL1 | −0.006920 |
| IFI35 | −0.006921 |
| SPRY4 | −0.006921 |
| TRIM16 | −0.006923 |
| HTR2A | −0.006923 |
| SYNGR2 | −0.006926 |
| SUMF1 | −0.006929 |
| TMEM232 | −0.006934 |
| CD83 | −0.006935 |
| ANKRD2 | −0.006937 |
| ST6GALNAC2 | −0.006945 |
| PRTG | −0.006947 |
| CYTH4 | −0.006953 |
| SH3BP5 | −0.006957 |
| RETSAT | −0.006957 |
| FMN1 | −0.006962 |
| MYH14 | −0.006967 |
| GBGT1 | −0.006969 |
| PRR29 | −0.006970 |
| CYP1B1 | −0.006971 |
| SPSB2 | −0.006973 |
| SP100 | −0.006973 |
| APOBEC3H | −0.006973 |
| SLC16A11 | −0.006974 |
| PDGFB | −0.006975 |
| EMILIN2 | −0.006983 |
| UBL3 | −0.006983 |
| LPAR5 | −0.006984 |
| CFAP46 | −0.006989 |
| P4HA2 | −0.006989 |
| GBP1 | −0.006994 |
| KCNK6 | −0.006997 |
| TXNIP | −0.007001 |
| GCHFR | −0.007001 |
| TRIBI | −0.007004 |
| IQSEC3 | −0.007007 |
| CHRM3 | −0.007008 |
| SMIM1 | −0.007014 |
| ANKRD45 | −0.007014 |
| PRNP | −0.007019 |
| IL1RAP | −0.007024 |
| FAM78A | −0.007026 |
| CEBPE | −0.007027 |
| OR2A7 | −0.007031 |
| QKI | −0.007034 |
| LGALS9C | −0.007034 |
| GPX1 | −0.007036 |
| SPIB | −0.007043 |
| TEAD2 | −0.007046 |
| CMTM7 | −0.007051 |
| ARHGEF5 | −0.007053 |
| ZBTB4 | −0.007054 |
| PON2 | −0.007058 |
| GAS6 | −0.007061 |
| TMEM106A | −0.007062 |
| CYP24A1 | −0.007066 |
| KIAA0040 | −0.007073 |
| NAT1 | −0.007074 |
| DAAM2 | −0.007076 |
| CARD11 | −0.007078 |
| INPP5D | −0.007080 |
| TIMD4 | −0.007082 |
| C20orf202 | −0.007083 |
| PM20D1 | −0.007085 |
| KDELR3 | −0.007085 |
| PAEP | −0.007086 |
| VASN | −0.007092 |
| ENTPD3 | −0.007093 |
| GNAI2 | −0.007096 |
| TMIE | −0.007098 |
| MT-ND6 | −0.007099 |
| CAST | −0.007100 |
| CREG1 | −0.007103 |
| RGL1 | −0.007110 |
| CST3 | −0.007115 |
| AMOTL2 | −0.007117 |
| NUPR1 | −0.007118 |
| 43718 | −0.007119 |
| MN1 | −0.007119 |
| P2RY1 | −0.007125 |
| RTKN2 | −0.007128 |
| MARVELD1 | −0.007130 |
| PPP1R42 | −0.007134 |
| BCL6B | −0.007134 |
| ABCB1 | −0.007138 |
| PREX2 | −0.007138 |
| AICDA | −0.007139 |
| FRMD4B | −0.007147 |
| EMILIN1 | −0.007160 |
| TRIM63 | −0.007160 |
| SUSD3 | −0.007165 |
| PLA2G3 | −0.007165 |
| CD80 | −0.007166 |
| PCDH1 | −0.007172 |
| ST6GALNAC6 | −0.007176 |
| MYD88 | −0.007178 |
| SLC46A3 | −0.007179 |
| TPP1 | −0.007180 |
| FUCA1 | −0.007181 |
| MLKL | −0.007187 |
| LRRC4 | −0.007192 |
| TGFB2 | −0.007192 |
| ST3GAL1 | −0.007193 |
| FN1 | −0.007194 |
| ABCD2 | −0.007196 |
| SLC2A9 | −0.007197 |
| IRX3 | −0.007203 |
| TNS3 | −0.007210 |
| SERPINB2 | −0.007210 |
| KCTD12 | −0.007211 |
| ABLIM3 | −0.007214 |
| ASB9 | −0.007215 |
| CSTB | −0.007221 |
| PLEKHF1 | −0.007224 |
| IFIT1 | −0.007227 |
| CD6 | −0.007228 |
| COLEC10 | −0.007229 |
| TACC1 | −0.007230 |
| TGFBI | −0.007232 |
| PIEZO1 | −0.007236 |
| TSPAN13 | −0.007242 |
| TMEM200B | −0.007242 |
| C5orf38 | −0.007244 |
| RTN4RL1 | −0.007253 |
| RASSF2 | −0.007256 |
| FZD7 | −0.007258 |
| MYC | −0.007258 |
| ZNF750 | −0.007260 |
| SERPINF1 | −0.007261 |
| CYP1A1 | −0.007264 |
| ATP6V0D2 | −0.007266 |
| UGT2B4 | −0.007267 |
| BCAM | −0.007268 |
| RASGRP2 | −0.007272 |
| DI03 | −0.007281 |
| KCNMB1 | −0.007285 |
| CRYM | −0.007286 |
| IGF2BP2 | −0.007288 |
| DEFA3 | −0.007291 |
| PGR | −0.007292 |
| PTK6 | −0.007293 |
| FLT3 | −0.007298 |
| EDEMI | −0.007298 |
| GATSL3 | −0.007299 |
| C10orf11 | −0.007300 |
| NOTCH2 | −0.007302 |
| CYGB | −0.007306 |
| ABCA6 | −0.007306 |
| KANK4 | −0.007312 |
| SERPINB6 | −0.007316 |
| WSCD2 | −0.007316 |
| FMOD | −0.007318 |
| RHOG | −0.007320 |
| ETV5 | −0.007320 |
| SP140 | −0.007321 |
| P2RY14 | −0.007325 |
| EPB41L2 | −0.007326 |
| STAT6 | −0.007329 |
| AFF2 | −0.007330 |
| KCNK5 | −0.007335 |
| ITM2B | −0.007339 |
| GALNT4 | −0.007342 |
| BLVRA | −0.007342 |
| PPP1R15A | −0.007342 |
| WFDC10B | −0.007344 |
| PLCXD3 | −0.007346 |
| LPCAT1 | −0.007350 |
| CORIN | −0.007356 |
| ALPK1 | −0.007357 |
| MAMLD1 | −0.007358 |
| KLF6 | −0.007358 |
| PRKCDBP | −0.007359 |
| XCL2 | −0.007360 |
| SLC9A9 | −0.007362 |
| TIMP2 | −0.007363 |
| NXPH3 | −0.007363 |
| MTUS1 | −0.007368 |
| KCNK15 | −0.007369 |
| LRRC23 | −0.007371 |
| SH3BGRL3 | −0.007371 |
| DOK7 | −0.007377 |
| TRIM71 | −0.007377 |
| SLC6A13 | −0.007378 |
| FYCO1 | −0.007379 |
| CCND3 | −0.007379 |
| GALNT18 | −0.007384 |
| TEX26 | −0.007387 |
| USHBP1 | −0.007390 |
| DUOX2 | −0.007391 |
| HLA-C | −0.007395 |
| ZFP36L2 | −0.007396 |
| ARHGAP6 | −0.007397 |
| GNLY | −0.007398 |
| PNMT | −0.007411 |
| CCR3 | −0.007412 |
| SH2D1A | −0.007412 |
| DNAJC15 | −0.007412 |
| CEBPD | −0.007413 |
| TMEM132C | −0.007414 |
| PRND | −0.007417 |
| KIAA1217 | −0.007421 |
| PHACTR2 | −0.007437 |
| SIRPD | −0.007441 |
| ELK3 | −0.007442 |
| MORN5 | −0.007445 |
| DNAH10 | −0.007445 |
| SERPINB9 | −0.007453 |
| KLRD1 | −0.007454 |
| DAPK1 | −0.007463 |
| PRKCB | −0.007466 |
| IL21R | −0.007467 |
| PEK3IP1 | −0.007471 |
| VEGFC | −0.007473 |
| SLC1A3 | −0.007473 |
| FAM129B | −0.007478 |
| PLA2G5 | −0.007478 |
| ARHGAP23 | −0.007485 |
| B4GALT1 | −0.007485 |
| TRPM4 | −0.007490 |
| SPG20 | −0.007494 |
| ATP11A | −0.007495 |
| TNFSF13B | −0.007496 |
| SMIM10 | −0.007500 |
| TAX1BP3 | −0.007501 |
| ARHGEF6 | −0.007501 |
| CSDC2 | −0.007513 |
| BAG3 | −0.007516 |
| IFNGR1 | −0.007519 |
| PCED1B | −0.007536 |
| DRC7 | −0.007538 |
| C9orf66 | −0.007542 |
| SNX33 | −0.007550 |
| CLEC1B | −0.007556 |
| CYP2A13 | −0.007556 |
| PRKCZ | −0.007558 |
| ANKDD1B | −0.007559 |
| AKAP7 | −0.007562 |
| IQCA1 | −0.007564 |
| PDE6G | −0.007564 |
| KCNJ8 | −0.007578 |
| TRIM61 | −0.007579 |
| SAMHD1 | −0.007579 |
| SH3BGRL | −0.007580 |
| KIR2DL3 | −0.007581 |
| LILRB4 | −0.007585 |
| CR2 | −0.007586 |
| RBM47 | −0.007586 |
| PRICKLE2 | −0.007592 |
| TMEM255A | −0.007594 |
| PDLIM1 | −0.007594 |
| INHBA | −0.007596 |
| ANKRD33B | −0.007599 |
| C7orf57 | −0.007600 |
| PIFO | −0.007602 |
| IL1R2 | −0.007602 |
| NCCRP1 | −0.007603 |
| NRROS | −0.007608 |
| VSTM1 | −0.007612 |
| TNF | −0.007612 |
| ENPP3 | −0.007614 |
| VENTX | −0.007633 |
| PXDC1 | −0.007635 |
| UPP1 | −0.007642 |
| LY96 | −0.007648 |
| EDIL3 | −0.007648 |
| GM2A | −0.007659 |
| TBX2 | −0.007662 |
| ITGB7 | −0.007669 |
| LBH | −0.007672 |
| AEBP1 | −0.007676 |
| TES | −0.007677 |
| PRR5L | −0.007677 |
| LAD1 | −0.007685 |
| PRKCE | −0.007688 |
| PYHIN1 | −0.007691 |
| ENPP2 | −0.007691 |
| FLNA | −0.007698 |
| RIN2 | −0.007699 |
| CLC | −0.007700 |
| TMEM144 | −0.007703 |
| GLUD2 | −0.007703 |
| COL14A1 | −0.007707 |
| CCDC85A | −0.007708 |
| RNF125 | −0.007708 |
| TNFAIP3 | −0.007708 |
| DLX3 | −0.007709 |
| RENBP | −0.007711 |
| GFPT2 | −0.007711 |
| NCR1 | −0.007714 |
| GLRX | −0.007716 |
| KCNA5 | −0.007721 |
| TPD52L1 | −0.007723 |
| LDOC1 | −0.007724 |
| KLB | −0.007730 |
| FAM46C | −0.007731 |
| RHOB | −0.007735 |
| FOSL2 | −0.007739 |
| TRAT1 | −0.007750 |
| NIM1K | −0.007751 |
| SLC7A8 | −0.007758 |
| GALNT6 | −0.007760 |
| LRAT | −0.007761 |
| SCNN1G | −0.007766 |
| MOCOS | −0.007769 |
| SLC24A4 | −0.007770 |
| CD59 | −0.007772 |
| ADAMTS15 | −0.007781 |
| CHRDL2 | −0.007783 |
| CYSTM1 | −0.007783 |
| TCN1 | −0.007785 |
| GPR34 | −0.007790 |
| CAMK2A | −0.007790 |
| METRNL | −0.007797 |
| JUNB | −0.007806 |
| TLR4 | −0.007811 |
| TAGLN | −0.007811 |
| NES | −0.007813 |
| CETP | −0.007818 |
| NTRK2 | −0.007820 |
| GLIS3 | −0.007821 |
| SIAE | −0.007826 |
| FYB | −0.007828 |
| SCD | −0.007830 |
| SAMD9L | −0.007835 |
| ZBTB16 | −0.007837 |
| BRINP3 | −0.007839 |
| C8orf48 | −0.007842 |
| RNASE6 | −0.007844 |
| ESYT3 | −0.007846 |
| OSM | −0.007846 |
| PLN | −0.007853 |
| PRKCD | −0.007854 |
| GPD1L | −0.007854 |
| ZDHHC19 | −0.007857 |
| CD84 | −0.007869 |
| RASIP1 | −0.007870 |
| KCTD11 | −0.007872 |
| B2M | −0.007880 |
| PDGFC | −0.007884 |
| IL7 | −0.007884 |
| ELOVL3 | −0.007885 |
| UNC93B1 | −0.007885 |
| NKD1 | −0.007892 |
| ENDOD1 | −0.007893 |
| PQLC3 | −0.007895 |
| SCARF1 | −0.007896 |
| PLIN3 | −0.007896 |
| IFITM1 | −0.007901 |
| TSPAN12 | −0.007903 |
| PITPNM3 | −0.007904 |
| CASP5 | −0.007904 |
| GRIN3B | −0.007916 |
| CLEC4A | −0.007919 |
| RARG | −0.007924 |
| QPRT | −0.007929 |
| LGALS1 | −0.007930 |
| RAPGEF3 | −0.007931 |
| BMPER | −0.007931 |
| HBG2 | −0.007932 |
| ROBO2 | −0.007933 |
| LGALS3BP | −0.007934 |
| TMC6 | −0.007939 |
| CTSL | −0.007939 |
| AK8 | −0.007940 |
| IFIT2 | −0.007953 |
| PLEKHG7 | −0.007968 |
| F2RL3 | −0.007978 |
| CYP39A1 | −0.007982 |
| GBP3 | −0.007983 |
| TFAP2C | −0.007984 |
| CFP | −0.007986 |
| APLP2 | −0.007991 |
| SLC28A3 | −0.007994 |
| EDNRA | −0.007995 |
| TKT | −0.007996 |
| PCDH12 | −0.007997 |
| ITGB5 | −0.008000 |
| TMEM243 | −0.008004 |
| IGSF6 | −0.008009 |
| GFHB | −0.008010 |
| CD8A | −0.008019 |
| KIR3DL1 | −0.008019 |
| TMEM159 | −0.008029 |
| CIB1 | −0.008031 |
| LY9 | −0.008033 |
| MAG | −0.008038 |
| CPQ | −0.008039 |
| CD274 | −0.008049 |
| DMKN | −0.008054 |
| TNFAIP8L3 | −0.008060 |
| CD247 | −0.008067 |
| ARHGEF35 | −0.008069 |
| MOB3B | −0.008071 |
| HEPH | −0.008072 |
| ITGA2 | −0.008073 |
| CD5 | −0.008076 |
| EPHB6 | −0.008077 |
| ERBB3 | −0.008077 |
| SLAMF1 | −0.008083 |
| REM1 | −0.008084 |
| VAV1 | −0.008088 |
| PCYOX1 | −0.008097 |
| HLA-F | −0.008099 |
| NFATC1 | −0.008102 |
| PDGFA | −0.008103 |
| TSPAN4 | −0.008106 |
| POLR2J2 | −0.008108 |
| PTH1R | −0.008110 |
| SLC14A1 | −0.008111 |
| ZC3H12A | −0.008115 |
| GIPC2 | −0.008119 |
| CDH15 | −0.008121 |
| GZMK | −0.008125 |
| SLC4A4 | −0.008126 |
| THBS1 | −0.008131 |
| IGLL5 | −0.008132 |
| TPK1 | −0.008134 |
| BCL9L | −0.008136 |
| DNAJC5B | −0.008136 |
| PSCA | −0.008136 |
| TGFBR3 | −0.008141 |
| SLC22A4 | −0.008141 |
| NRP2 | −0.008147 |
| CD300LB | −0.008150 |
| C19orf35 | −0.008150 |
| CPA6 | −0.008152 |
| LRRC31 | −0.008153 |
| LAMA2 | −0.008154 |
| CRTAP | −0.008158 |
| TNFSF8 | −0.008174 |
| EGR3 | −0.008185 |
| C15orf52 | −0.008196 |
| ASS1 | −0.008198 |
| RAI2 | −0.008198 |
| CITED2 | −0.008201 |
| TCL1A | −0.008201 |
| CCDC80 | −0.008202 |
| GHR | −0.008203 |
| ANGPTL7 | −0.008205 |
| MAML2 | −0.008210 |
| FCRL6 | −0.008224 |
| SFN | −0.008224 |
| RERG | −0.008231 |
| SLAMF8 | −0.008235 |
| GZMA | −0.008236 |
| RNF175 | −0.008237 |
| CDC42EP5 | −0.008242 |
| HRH1 | −0.008247 |
| GPR171 | −0.008248 |
| ANXA11 | −0.008253 |
| CASP12 | −0.008261 |
| FAM181A | −0.008262 |
| ITPRIP | −0.008262 |
| C2orf15 | −0.008263 |
| CD63 | −0.008263 |
| TALI | −0.008269 |
| SLFN5 | −0.008272 |
| RMDN2 | −0.008279 |
| IFIT3 | −0.008284 |
| SPAG6 | −0.008284 |
| BCL2L14 | −0.008286 |
| HYAL2 | −0.008289 |
| VSTM4 | −0.008289 |
| CYB5A | −0.008290 |
| IL1RL2 | −0.008295 |
| KLHL6 | −0.008299 |
| TMEM150A | −0.008301 |
| AOAH | −0.008302 |
| VNN1 | −0.008308 |
| EGFL7 | −0.008308 |
| ARSD | −0.008309 |
| MT1E | −0.008314 |
| RARA | −0.008316 |
| CTSB | −0.008320 |
| BHLHE40 | −0.008322 |
| GNA14 | −0.008323 |
| MYL12A | −0.008326 |
| IL17RE | −0.008329 |
| TEKT4 | −0.008341 |
| CAPN9 | −0.008343 |
| GSTM1 | −0.008344 |
| FCRL1 | −0.008347 |
| TFEC | −0.008352 |
| ISLR | −0.008352 |
| PLA2G2D | −0.008364 |
| CPXM2 | −0.008365 |
| DDR2 | −0.008365 |
| LILRB1 | −0.008368 |
| JPH2 | −0.008370 |
| GPC3 | −0.008372 |
| CCR7 | −0.008373 |
| CD151 | −0.008375 |
| AMPD1 | −0.008381 |
| ABCC4 | −0.008382 |
| RAB27A | −0.008389 |
| KCNA3 | −0.008392 |
| HLA-B | −0.008398 |
| RARRES1 | −0.008403 |
| FLNC | −0.008408 |
| CYP4F22 | −0.008413 |
| PACSIN3 | −0.008414 |
| GATA2 | −0.008417 |
| C19orf38 | −0.008421 |
| PPIC | −0.008423 |
| BCL3 | −0.008423 |
| PILRA | −0.008426 |
| SPRED1 | −0.008431 |
| DUSP5 | −0.008433 |
| KL | −0.008442 |
| MATK | −0.008444 |
| NKD2 | −0.008445 |
| ABCA13 | −0.008445 |
| RERGL | −0.008448 |
| C15orf59 | −0.008453 |
| SFRP4 | −0.008461 |
| TAGLN2 | −0.008483 |
| FGL2 | −0.008485 |
| RNF144B | −0.008488 |
| CYTL1 | −0.008489 |
| RAB44 | −0.008491 |
| LMCD1 | −0.008494 |
| LTA4H | −0.008497 |
| SMTNL2 | −0.008498 |
| GP9 | −0.008503 |
| ADRA1B | −0.008504 |
| HCLS1 | −0.008508 |
| SEMA5A | −0.008509 |
| THSD4 | −0.008510 |
| NAGS | −0.008513 |
| MEFV | −0.008515 |
| TMEM252 | −0.008519 |
| FAXDC2 | −0.008520 |
| ICAM3 | −0.008525 |
| THSD1 | −0.008525 |
| COMT | −0.008526 |
| MUC22 | −0.008527 |
| PRKG1 | −0.008530 |
| RCSD1 | −0.008532 |
| TGFA | −0.008534 |
| PCSK5 | −0.008538 |
| ATP13A5 | −0.008543 |
| TRIP6 | −0.008544 |
| CHTA | −0.008548 |
| SMPDL3B | −0.008551 |
| CXorf36 | −0.008557 |
| CGNL1 | −0.008557 |
| ERG | −0.008559 |
| AARD | −0.008560 |
| FBXO27 | −0.008561 |
| DEFA1B | −0.008562 |
| KLHDC7A | −0.008565 |
| EHD1 | −0.008568 |
| CXCL14 | −0.008572 |
| SH3D19 | −0.008573 |
| SPRY2 | −0.008575 |
| ARL6IP5 | −0.008577 |
| DYSF | −0.008577 |
| ACY3 | −0.008578 |
| MILR1 | −0.008580 |
| FAM110D | −0.008583 |
| EFCAB1 | −0.008583 |
| SLC25A23 | −0.008583 |
| KRT17 | −0.008588 |
| TBX21 | −0.008591 |
| TRIM5 | −0.008592 |
| IL10RA | −0.008596 |
| AGRN | −0.008596 |
| CLEC2B | −0.008599 |
| RXRA | −0.008600 |
| FBXO2 | −0.008600 |
| VWA5A | −0.008604 |
| RASSF8 | −0.008614 |
| BOK | −0.008618 |
| EDAR | −0.008621 |
| ST3GAL5 | −0.008622 |
| MAP3K19 | −0.008626 |
| CD96 | −0.008628 |
| TMEM204 | −0.008628 |
| CD109 | −0.008633 |
| SLC2A3 | −0.008638 |
| C15orf48 | −0.008642 |
| BATF2 | −0.008644 |
| WFDC6 | −0.008645 |
| ATP12A | −0.008649 |
| MFAP5 | −0.008658 |
| SLC10A2 | −0.008662 |
| LPXN | −0.008662 |
| PDE1B | −0.008665 |
| LMOD3 | −0.008667 |
| MGAT3 | −0.008677 |
| TNNT3 | −0.008677 |
| GPR87 | −0.008681 |
| TREML1 | −0.008682 |
| FAS | −0.008683 |
| DSC2 | −0.008697 |
| GRK5 | −0.008698 |
| LPCAT2 | −0.008698 |
| COL13A1 | −0.008699 |
| CCL11 | −0.008703 |
| DLEC1 | −0.008706 |
| TINAGL1 | −0.008707 |
| TCTEX1D1 | −0.008707 |
| IL2RB | −0.008709 |
| APOBEC4 | −0.008710 |
| YBX3 | −0.008711 |
| RNASE2 | −0.008712 |
| GBP2 | −0.008715 |
| SAMD4A | −0.008715 |
| GSN | −0.008717 |
| CYBA | −0.008718 |
| TNFRSF10D | −0.008720 |
| CDKL2 | −0.008724 |
| ABI3 | −0.008726 |
| DENND2C | −0.008729 |
| KLF5 | −0.008737 |
| KCNS3 | −0.008738 |
| ALAS2 | −0.008739 |
| SAMSN1 | −0.008740 |
| CCDC102A | −0.008743 |
| SPINK1 | −0.008746 |
| CLCF1 | −0.008752 |
| CD207 | −0.008754 |
| LRRC52 | −0.008755 |
| RFTN1 | −0.008756 |
| LIPA | −0.008760 |
| PRSS35 | −0.008762 |
| ITK | −0.008763 |
| PTPRCAP | −0.008763 |
| ARMC4 | −0.008766 |
| ACSS2 | −0.008769 |
| CP | −0.008771 |
| SSC5D | −0.008776 |
| LTB | −0.008778 |
| ADH1A | −0.008779 |
| C6orf229 | −0.008781 |
| HPR | −0.008787 |
| L1TD1 | −0.008795 |
| LRP1 | −0.008796 |
| FREM2 | −0.008804 |
| NCEH1 | −0.008805 |
| THEMIS | −0.008808 |
| FUOM | −0.008809 |
| LRP10 | −0.008811 |
| LCN6 | −0.008814 |
| PLSCR4 | −0.008816 |
| AGPAT2 | −0.008818 |
| MYLK | −0.008828 |
| SLC25A43 | −0.008829 |
| NDRG2 | −0.008829 |
| ZCCHC24 | −0.008832 |
| PIK3R5 | −0.008833 |
| CLEC4D | −0.008837 |
| TM6SF1 | −0.008838 |
| ME1 | −0.008844 |
| LRRC36 | −0.008845 |
| CAV3 | −0.008847 |
| TCN2 | −0.008850 |
| PTGS1 | −0.008851 |
| LCP2 | −0.008855 |
| DEFA1 | −0.008863 |
| ACE | −0.008866 |
| DOK4 | −0.008870 |
| IGFBP6 | −0.008877 |
| IRF8 | −0.008879 |
| PLAC8 | −0.008879 |
| PDK4 | −0.008882 |
| P2RY10 | −0.008884 |
| GPR132 | −0.008885 |
| EFEMP2 | −0.008887 |
| TRIM29 | −0.008887 |
| TLL1 | −0.008887 |
| SLC16A12 | −0.008897 |
| ARPC1B | −0.008898 |
| BCHE | −0.008902 |
| FPR3 | −0.008903 |
| LYPD2 | −0.008908 |
| GPC4 | −0.008911 |
| MYO1C | −0.008914 |
| PPL | −0.008914 |
| IL5RA | −0.008917 |
| HPD | −0.008926 |
| CELA2B | −0.008927 |
| SYNE1 | −0.008931 |
| TNFRSF10A | −0.008932 |
| KLF9 | −0.008933 |
| IL15RA | −0.008937 |
| ZNF185 | −0.008941 |
| IER3 | −0.008943 |
| GNG7 | −0.008945 |
| APOL3 | −0.008950 |
| KDR | −0.008952 |
| LILRB2 | −0.008957 |
| CHP1 | −0.008962 |
| EPHX3 | −0.008963 |
| TMEM105 | −0.008964 |
| CTSO | −0.008975 |
| TSPAN1 | −0.008975 |
| GPSM3 | −0.008981 |
| CD164L2 | −0.008981 |
| MPEG1 | −0.008982 |
| SMAGP | −0.008989 |
| AK1 | −0.008991 |
| FEZ1 | −0.008993 |
| BMX | −0.008999 |
| BMPR1B | −0.009003 |
| APLNR | −0.009007 |
| NRGN | −0.009007 |
| PDE4D | −0.009009 |
| SPNS3 | −0.009009 |
| ATP6V0A4 | −0.009011 |
| ITGA5 | −0.009016 |
| ADORA2B | −0.009019 |
| PLA2G2A | −0.009027 |
| TAGAP | −0.009029 |
| DHCR24 | −0.009043 |
| EPHA1 | −0.009044 |
| CECR1 | −0.009046 |
| GZMM | −0.009046 |
| PRRG1 | −0.009061 |
| CYYR1 | −0.009062 |
| HAS2 | −0.009063 |
| CLMP | −0.009066 |
| KMO | −0.009066 |
| BTNL9 | −0.009068 |
| SLAMF6 | −0.009072 |
| NBL1 | −0.009072 |
| PLAUR | −0.009073 |
| SYNPO2 | −0.009078 |
| GLI3 | −0.009078 |
| ZNF831 | −0.009079 |
| SPDEF | −0.009079 |
| DNAH12 | −0.009082 |
| FMO5 | −0.009085 |
| GPR146 | −0.009090 |
| ZNF366 | −0.009093 |
| STXBP6 | −0.009093 |
| XPNPEP2 | −0.009095 |
| ZFPM2 | −0.009098 |
| TRIM31 | −0.009103 |
| WBSCR17 | −0.009107 |
| ACSM3 | −0.009108 |
| TM4SF4 | −0.009109 |
| HAS3 | −0.009115 |
| ITPKC | −0.009116 |
| NKAPL | −0.009118 |
| RIN3 | −0.009119 |
| RAB20 | −0.009124 |
| CD81 | −0.009133 |
| TMED6 | −0.009135 |
| NOG | −0.009136 |
| NMUR1 | −0.009138 |
| MLC1 | −0.009142 |
| TJP3 | −0.009148 |
| FGFR3 | −0.009150 |
| CYP2A6 | −0.009152 |
| HGF | −0.009153 |
| PLCD1 | −0.009160 |
| KHDRBS2 | −0.009177 |
| BIN2 | −0.009178 |
| EFNA1 | −0.009178 |
| DKK3 | −0.009178 |
| TRIM22 | −0.009181 |
| CDH11 | −0.009184 |
| MFNG | −0.009186 |
| PTCHD1 | −0.009186 |
| RAB32 | −0.009188 |
| IL20RA | −0.009192 |
| LMNA | −0.009196 |
| SLC18A2 | −0.009206 |
| KIR3DL2 | −0.009206 |
| GAS1 | −0.009212 |
| LIF | −0.009212 |
| TOR4A | −0.009212 |
| CFH | −0.009227 |
| GBP4 | −0.009230 |
| FMO4 | −0.009231 |
| PDGFRA | −0.009233 |
| NHLRC4 | −0.009236 |
| GIMAP2 | −0.009241 |
| EPHX1 | −0.009243 |
| ELN | −0.009264 |
| TSKS | −0.009269 |
| MTRNR2L12 | −0.009275 |
| RASL11A | −0.009278 |
| EIF4EBP3 | −0.009280 |
| GPR157 | −0.009284 |
| SLC9A3R2 | −0.009294 |
| ACOXL | −0.009294 |
| IL34 | −0.009295 |
| EPHB4 | −0.009297 |
| CTSZ | −0.009297 |
| CD40 | −0.009301 |
| CPZ | −0.009306 |
| ANGPTL5 | −0.009308 |
| NOD2 | −0.009309 |
| SH2D3C | −0.009310 |
| CDK15 | −0.009312 |
| PLEKHO2 | −0.009320 |
| MAP3K8 | −0.009321 |
| SLC52A1 | −0.009333 |
| ABCG2 | −0.009336 |
| CCIN | −0.009337 |
| WTIP | −0.009349 |
| B4GALNT2 | −0.009351 |
| TMC8 | −0.009354 |
| ANKFN1 | −0.009361 |
| CMKLR1 | −0.009362 |
| GPR183 | −0.009363 |
| C9orf135 | −0.009369 |
| CRYAB | −0.009370 |
| ITGBL1 | −0.009373 |
| IGF1 | −0.009383 |
| FBLN2 | −0.009384 |
| DAPK2 | −0.009390 |
| FOLR2 | −0.009396 |
| SOCS2 | −0.009400 |
| ITPR1 | −0.009401 |
| RGS18 | −0.009410 |
| ST8SIA1 | −0.009420 |
| MAPKAPK3 | −0.009423 |
| KLF15 | −0.009427 |
| FILIP1L | −0.009430 |
| SH2D1B | −0.009433 |
| HAS1 | −0.009435 |
| HIF3A | −0.009436 |
| PLTP | −0.009445 |
| KRT1 | −0.009451 |
| LY75 | −0.009452 |
| FCGRT | −0.009452 |
| SCARA3 | −0.009453 |
| ERICH2 | −0.009453 |
| AWAT2 | −0.009457 |
| C5AR2 | −0.009463 |
| MUC4 | −0.009467 |
| CD3D | −0.009469 |
| S100B | −0.009471 |
| LRRC15 | −0.009476 |
| IL16 | −0.009476 |
| VILL | −0.009488 |
| CYP11A1 | −0.009488 |
| NOSTRIN | −0.009490 |
| AHNAK2 | −0.009494 |
| KLF17 | −0.009499 |
| RPS6KA2 | −0.009499 |
| MUC16 | −0.009502 |
| KLF8 | −0.009504 |
| APOBEC3C | −0.009505 |
| GATA1 | −0.009506 |
| CDO1 | −0.009509 |
| MMRN2 | −0.009516 |
| CD7 | −0.009518 |
| FGFRL1 | −0.009524 |
| KLRF1 | −0.009530 |
| PLG | −0.009539 |
| MECOM | −0.009540 |
| FBXO17 | −0.009542 |
| ROPN1L | −0.009542 |
| BANK1 | −0.009544 |
| CXorf21 | −0.009550 |
| BST1 | −0.009551 |
| DYNLRB2 | −0.009552 |
| TNFRSF10B | −0.009555 |
| PIK3CG | −0.009556 |
| SGK1 | −0.009558 |
| PIP5K1B | −0.009558 |
| RXRG | −0.009559 |
| LRRC32 | −0.009563 |
| LRRN4CL | −0.009563 |
| FTL | −0.009563 |
| CCR5 | −0.009564 |
| HVCN1 | −0.009571 |
| PTPN14 | −0.009579 |
| DOCK2 | −0.009590 |
| CD34 | −0.009599 |
| PLD4 | −0.009600 |
| DAPP1 | −0.009604 |
| GLDN | −0.009615 |
| ARL11 | −0.009615 |
| NFAM1 | −0.009616 |
| C4A | −0.009620 |
| CRIM1 | −0.009621 |
| NFKBIA | −0.009631 |
| FAT4 | −0.009633 |
| RAB11FIP1 | −0.009639 |
| WAS | −0.009652 |
| VWA3A | −0.009656 |
| C9orf152 | −0.009658 |
| GPER1 | −0.009662 |
| NPY4R | −0.009672 |
| MGP | −0.009676 |
| FAH | −0.009679 |
| C10orf10 | −0.009682 |
| TSPAN32 | −0.009685 |
| OSR1 | −0.009685 |
| NLRP3 | −0.009685 |
| PRDM6 | −0.009686 |
| FAM180A | −0.009690 |
| CPVL | −0.009692 |
| FERMT3 | −0.009696 |
| ABCA4 | −0.009697 |
| ICAM2 | −0.009704 |
| AQP10 | −0.009706 |
| RAMP2 | −0.009707 |
| SLC6A12 | −0.009721 |
| SEMA3C | −0.009730 |
| TGFB1 | −0.009731 |
| NCF4 | −0.009736 |
| PIH1D3 | −0.009736 |
| POU2F3 | −0.009737 |
| MET | −0.009738 |
| CAT | −0.009738 |
| HMOX1 | −0.009739 |
| EPS8L1 | −0.009743 |
| NCKAP5 | −0.009749 |
| NPIPA5 | −0.009751 |
| ITGAX | −0.009752 |
| ADAP1 | −0.009758 |
| ADRA1D | −0.009768 |
| THEMIS2 | −0.009779 |
| HFE | −0.009780 |
| PTPN22 | −0.009792 |
| CDH19 | −0.009793 |
| TRPV4 | −0.009796 |
| SERPINB3 | −0.009798 |
| BATF | −0.009804 |
| LEPR | −0.009810 |
| GNA15 | −0.009812 |
| BHLHE41 | −0.009814 |
| RAB37 | −0.009819 |
| NR4A1 | −0.009820 |
| OTC | −0.009830 |
| CWH43 | −0.009833 |
| GSDMC | −0.009837 |
| HLA-DOB | −0.009840 |
| TNFRSF8 | −0.009843 |
| ELANE | −0.009844 |
| TSPAN18 | −0.009845 |
| CDKL1 | −0.009868 |
| ZFP36L1 | −0.009869 |
| VNN3 | −0.009869 |
| LRRC8E | −0.009870 |
| GBP6 | −0.009874 |
| TBXAS1 | −0.009877 |
| SORCS2 | −0.009878 |
| C1orf141 | −0.009882 |
| KLHL30 | −0.009885 |
| ADAMTSL5 | −0.009885 |
| LRRC71 | −0.009888 |
| IKZF1 | −0.009897 |
| HSD11B1 | −0.009900 |
| FAM124B | −0.009903 |
| LRRC2 | −0.009904 |
| BPIFA1 | −0.009906 |
| LYN | −0.009910 |
| MGST1 | −0.009913 |
| MYO1G | −0.009920 |
| ERBB2 | −0.009924 |
| TMEM71 | −0.009926 |
| CRIP1 | −0.009938 |
| JDP2 | −0.009938 |
| PYCARD | −0.009950 |
| LPAR3 | −0.009967 |
| CCL19 | −0.009967 |
| C3orf36 | −0.009971 |
| IL12RB1 | −0.009975 |
| CASP10 | −0.009976 |
| PTPN21 | −0.009982 |
| F13A1 | −0.009982 |
| LILRB3 | −0.009986 |
| 1L27RA | −0.009988 |
| LIMCH1 | −0.009990 |
| ARHGAP44 | −0.009990 |
| SPARCL1 | −0.009991 |
| PLEK | −0.009993 |
| CD2 | −0.009994 |
| MICA | −0.009997 |
| CD14 | −0.009997 |
| AKAP14 | −0.010000 |
| WDR63 | −0.010002 |
| SLCO4C1 | −0.010005 |
| OMG | −0.010011 |
| TST | −0.010015 |
| SSPN | −0.010018 |
| VSNL1 | −0.010024 |
| SASH3 | −0.010026 |
| SERPINA9 | −0.010028 |
| MOXD1 | −0.010031 |
| ECHDC3 | −0.010034 |
| SERTAD1 | −0.010035 |
| EGFR | −0.010046 |
| FCAR | −0.010051 |
| CCND2 | −0.010061 |
| CD302 | −0.010065 |
| MISP | −0.010074 |
| RIPK3 | −0.010076 |
| WNT7B | −0.010079 |
| CYTIP | −0.010079 |
| TNFRSF1B | −0.010084 |
| IFI27 | −0.010086 |
| FXYD4 | −0.010087 |
| CD48 | −0.010090 |
| TLR5 | −0.010093 |
| LM03 | −0.010100 |
| OAS2 | −0.010104 |
| AIF1 | −0.010108 |
| LCP1 | −0.010122 |
| PLA2G16 | −0.010125 |
| LAMB2 | −0.010131 |
| EVI2A | −0.010139 |
| CFB | −0.010143 |
| TMC5 | −0.010146 |
| LPL | −0.010146 |
| ARHGAP18 | −0.010148 |
| BDNF | −0.010149 |
| CRLF2 | −0.010159 |
| IL4R | −0.010159 |
| C4B | −0.010161 |
| IL18R1 | −0.010166 |
| C1R | −0.010166 |
| CEACAM3 | −0.010169 |
| GJB3 | −0.010169 |
| CAPN6 | −0.010170 |
| GIMAP4 | −0.010174 |
| SIRPA | −0.010183 |
| CGN | −0.010187 |
| TNFSF10 | −0.010190 |
| FCGR1B | −0.010192 |
| CCL15 | −0.010194 |
| C1orf189 | −0.010208 |
| GGT1 | −0.010212 |
| RASL12 | −0.010224 |
| FM03 | −0.010226 |
| GOLT1A | −0.010229 |
| TNFAIP8L2 | −0.010231 |
| PALM3 | −0.010231 |
| HAVCR2 | −0.010232 |
| CPNE8 | −0.010241 |
| GZMB | −0.010244 |
| ADAMTS1 | −0.010255 |
| CD244 | −0.010259 |
| PTGFR | −0.010262 |
| P2RX2 | −0.010274 |
| SHC3 | −0.010282 |
| CAI2 | −0.010284 |
| C18orf63 | −0.010285 |
| TNFRSF1A | −0.010291 |
| GPNMB | −0.010293 |
| ENKUR | −0.010297 |
| DIIRS3 | −0.010298 |
| PLCD3 | −0.010298 |
| HACD4 | −0.010298 |
| PTPRC | −0.010300 |
| HIGD1B | −0.010300 |
| CD3G | −0.010312 |
| COPZ2 | −0.010318 |
| PALMD | −0.010318 |
| AD1I7 | −0.010320 |
| IL3RA | −0.010330 |
| ILI3RA2 | −0.010332 |
| C10orf67 | −0.010333 |
| FAM20A | −0.010333 |
| FAM83A | −0.010338 |
| ARHGAP40 | −0.010339 |
| ZNF670 | −0.010345 |
| TMEM163 | −0.010351 |
| GRN | −0.010355 |
| LPAR1 | −0.010357 |
| ACTN2 | −0.010359 |
| BCAT1 | −0.010360 |
| PLS3 | −0.010362 |
| SIGLEC9 | −0.010366 |
| WFDC2 | −0.010368 |
| FSTL3 | −0.010382 |
| ART4 | −0.010389 |
| B3GNT8 | −0.010392 |
| MFSD7 | −0.010396 |
| ADRA1A | −0.010403 |
| C11orf96 | −0.010410 |
| GIMAP5 | −0.010413 |
| LGALS9 | −0.010418 |
| HMGCS2 | −0.010421 |
| P2RX1 | −0.010422 |
| CLEC6A | −0.010438 |
| NLRC4 | −0.010447 |
| PPP1R14A | −0.010448 |
| SLC31A2 | −0.010457 |
| AKAP2 | −0.010461 |
| BIRC3 | −0.010464 |
| FAM92B | −0.010472 |
| TCF7L1 | −0.010477 |
| LIPN | −0.010481 |
| TPPP3 | −0.010481 |
| IVL | −0.010486 |
| TNFSF14 | −0.010488 |
| PERI | −0.010488 |
| CLEC7A | −0.010492 |
| GIMAP7 | −0.010493 |
| DUSP6 | −0.010494 |
| MUM1L1 | −0.010494 |
| RBMS2 | −0.010506 |
| SPINK5 | −0.010508 |
| SLC40A1 | −0.010509 |
| ASAHI | −0.010520 |
| TWIST2 | −0.010524 |
| PLEKHS1 | −0.010526 |
| ARHGAP30 | −0.010532 |
| SLIT2 | −0.010532 |
| LILRA1 | −0.010532 |
| CAPN13 | −0.010534 |
| RILPL2 | −0.010534 |
| ASPG | −0.010536 |
| APOL1 | −0.010537 |
| GPR65 | −0.010539 |
| VNN2 | −0.010539 |
| HMCN1 | −0.010546 |
| FAM150B | −0.010555 |
| PENK | −0.010558 |
| MFRP | −0.010560 |
| TNFRSF12A | −0.010573 |
| MT1L | −0.010576 |
| MYO1F | −0.010578 |
| PROCR | −0.010581 |
| DPYD | −0.010592 |
| QSOX1 | −0.010594 |
| CMA1 | −0.010598 |
| VIM | −0.010599 |
| ICOS | −0.010600 |
| PTPN13 | −0.010601 |
| ROR1 | −0.010607 |
| LAMC3 | −0.010613 |
| MASP1 | −0.010614 |
| CLEC9A | −0.010616 |
| TRIM6 | −0.010616 |
| CLEC1A | −0.010625 |
| COX7A1 | −0.010625 |
| CLDN1 | −0.010626 |
| TM4SF1 | −0.010636 |
| TMEM173 | −0.010636 |
| MYOCD | −0.010637 |
| VDR | −0.010651 |
| ARAP2 | −0.010654 |
| SIGLEC7 | −0.010666 |
| KLF4 | −0.010668 |
| FUT3 | −0.010668 |
| TESPA1 | −0.010670 |
| C11orf21 | −0.010678 |
| TSPO | −0.010680 |
| PLA2G4E | −0.010683 |
| CLDN23 | −0.010687 |
| HSPB2 | −0.010696 |
| ALS2CL | −0.010699 |
| GJA1 | −0.010700 |
| MYCT1 | −0.010709 |
| SPATAI8 | −0.010711 |
| KCNQ1 | −0.010713 |
| FCGR2B | −0.010714 |
| SDC1 | −0.010716 |
| RXFP1 | −0.010722 |
| EVA1A | −0.010724 |
| LRRC25 | −0.010724 |
| FBLN1 | −0.010726 |
| SMAD6 | −0.010732 |
| FOXN1 | −0.010741 |
| NCF1 | −0.010742 |
| HSPA12B | −0.010746 |
| OLFML3 | −0.010751 |
| PKNOX2 | −0.010755 |
| PTPRE | −0.010762 |
| C1S | −0.010766 |
| MYOZ1 | −0.010766 |
| EDN3 | −0.010768 |
| STARD8 | −0.010769 |
| MGLL | −0.010770 |
| KIF1C | −0.010770 |
| MRC2 | −0.010772 |
| SPOCD1 | −0.010772 |
| ANXA4 | −0.010780 |
| MDFIC | −0.010780 |
| CSF2RB | −0.010783 |
| SNTB1 | −0.010784 |
| PADI4 | −0.010788 |
| GNGT2 | −0.010792 |
| SELL | −0.010795 |
| ARRB1 | −0.010801 |
| JAM2 | −0.010803 |
| LEFTY2 | −0.010809 |
| GSTP1 | −0.010811 |
| MR1 | −0.010811 |
| NTNG1 | −0.010817 |
| ACKR4 | −0.010823 |
| RHOF | −0.010827 |
| ZNF365 | −0.010827 |
| SCN4B | −0.010830 |
| CD82 | −0.010834 |
| MIA2 | −0.010838 |
| TPRG1 | −0.010845 |
| SIGLEC1 | −0.010845 |
| BST2 | −0.010845 |
| MYH1 | −0.010846 |
| CD9 | −0.010856 |
| FCER2 | −0.010857 |
| TRIM34 | −0.010862 |
| ATP2C2 | −0.010869 |
| PF4 | −0.010869 |
| CA3 | −0.010871 |
| MPP7 | −0.010875 |
| HRASLS2 | −0.010875 |
| TMEM236 | −0.010877 |
| MSRB3 | −0.010880 |
| DAB2 | −0.010889 |
| GAS7 | −0.010897 |
| P2RY13 | −0.010899 |
| ATP10A | −0.010900 |
| MLPH | −0.010901 |
| CCR4 | −0.010907 |
| OMD | −0.010910 |
| CD38 | −0.010919 |
| IGSF10 | −0.010921 |
| TRHDE | −0.010923 |
| CXCL12 | −0.010929 |
| CSF1 | −0.010932 |
| CYP3A7 | −0.010933 |
| RNF223 | −0.010940 |
| LDLR | −0.010943 |
| CCL2 | −0.010944 |
| CDA | −0.010944 |
| LAMA3 | −0.010954 |
| CCDC141 | −0.010959 |
| COL8A1 | −0.010963 |
| GZMH | −0.010965 |
| CXCR3 | −0.010967 |
| NEXN | −0.010980 |
| MSN | −0.010986 |
| WFS1 | −0.010988 |
| RGN | −0.011001 |
| CLDN8 | −0.011001 |
| WFDC1 | −0.011005 |
| HNMT | −0.011008 |
| TRPC6 | −0.011014 |
| CTSC | −0.011021 |
| ARHGDIB | −0.011026 |
| GNG11 | −0.011027 |
| ZNF385B | −0.011028 |
| ALDH1A2 | −0.011033 |
| SLC5A9 | −0.011038 |
| MME | −0.011039 |
| CEACAM4 | −0.011050 |
| ACTG2 | −0.011052 |
| CATIP | −0.011055 |
| DUSP27 | −0.011061 |
| C3AR1 | −0.011062 |
| CR1 | −0.011063 |
| ALDH1A1 | −0.011065 |
| CRTAM | −0.011066 |
| ILIB | −0.011067 |
| FAM166B | −0.011069 |
| LY6E | −0.011073 |
| PLXDC2 | −0.011076 |
| RHOD | −0.011081 |
| PID1 | −0.011103 |
| KLF2 | −0.011105 |
| CD86 | −0.011108 |
| FCGR1A | −0.011112 |
| ARHGEF15 | −0.011112 |
| EGFL6 | −0.011118 |
| SPON1 | −0.011121 |
| OLFML1 | −0.011126 |
| ADCY7 | −0.011135 |
| SLC15A2 | −0.011137 |
| CCR1 | −0.011138 |
| SOX7 | −0.011138 |
| ACOX2 | −0.011139 |
| GGT2 | −0.011152 |
| WWTR1 | −0.011159 |
| COX4I2 | −0.011163 |
| CELF2 | −0.011163 |
| ADAMTSL3 | −0.011164 |
| KLHL4 | −0.011166 |
| MYL3 | −0.011184 |
| THRB | −0.011193 |
| FAM89A | −0.011200 |
| RBPMS | −0.011202 |
| GYPC | −0.011206 |
| C4BPB | −0.011207 |
| CXCL1 | −0.011220 |
| FXYD5 | −0.011221 |
| APBB1IP | −0.011221 |
| PTGER2 | −0.011222 |
| HTR3C | −0.011223 |
| DOCK8 | −0.011229 |
| VSIG1 | −0.011232 |
| CACNA2D2 | −0.011233 |
| NT5E | −0.011244 |
| CA2 | −0.011245 |
| VWA3B | −0.011245 |
| HACD1 | −0.011245 |
| CYP27A1 | −0.011246 |
| STOM | −0.011249 |
| LATS2 | −0.011249 |
| CD3E | −0.011257 |
| CLDN16 | −0.011260 |
| PSAPL1 | −0.011260 |
| ASPA | −0.011261 |
| TIE1 | −0.011270 |
| MITF | −0.011274 |
| PRSS22 | −0.011287 |
| RAB38 | −0.011290 |
| ZNF683 | −0.011307 |
| FABP3 | −0.011310 |
| CYP17A1 | −0.011312 |
| SNCG | −0.011317 |
| IFITM2 | −0.011319 |
| ARSI | −0.011321 |
| GIMAP6 | −0.011324 |
| GALM | −0.011334 |
| IL32 | −0.011339 |
| LUM | −0.011345 |
| GO2 | −0.011348 |
| LSP1 | −0.011360 |
| PQLC2L | −0.011366 |
| IFI30 | −0.011368 |
| HK3 | −0.011369 |
| CSF1R | −0.011372 |
| CD101 | −0.011381 |
| CASP1 | −0.011389 |
| PPP1R14C | −0.011396 |
| TLR7 | −0.011397 |
| RAB31 | −0.011399 |
| DTX4 | −0.011400 |
| SPNS2 | −0.011403 |
| KANK3 | −0.011404 |
| MS4A4A | −0.011417 |
| BNIPL | −0.011422 |
| LILRA6 | −0.011424 |
| BMP4 | −0.011425 |
| EGR2 | −0.011426 |
| TNFSF12 | −0.011431 |
| CDH5 | −0.011432 |
| KRT19 | −0.011432 |
| GIMAP1 | −0.011433 |
| CLEC5A | −0.011434 |
| CLIC6 | −0.011435 |
| FOS | −0.011437 |
| NKG7 | −0.011438 |
| MUSK | −0.011441 |
| CCL5 | −0.011452 |
| GMFG | −0.011456 |
| SELE | −0.011460 |
| CLEC14A | −0.011467 |
| TMPRSS13 | −0.011467 |
| ESAM | −0.011469 |
| ADPRH | −0.011472 |
| IL18 | −0.011473 |
| KIAA1462 | −0.011473 |
| SCUBE1 | −0.011476 |
| TDRD10 | −0.011479 |
| FHL5 | −0.011481 |
| FCGR3A | −0.011489 |
| SPATS1 | −0.011489 |
| PDLIM2 | −0.011492 |
| SERTM1 | −0.011499 |
| SEMA3B | −0.011502 |
| CFAP52 | −0.011511 |
| DNASE1L3 | −0.011513 |
| CEACAM8 | −0.011515 |
| CD93 | −0.011516 |
| GAS2L2 | −0.011518 |
| EPDR1 | −0.011520 |
| MMP2 | −0.011522 |
| CACNG6 | −0.011525 |
| RND1 | −0.011526 |
| VWF | −0.011529 |
| SLC44A4 | −0.011531 |
| TNFRSF10C | −0.011541 |
| SOX17 | −0.011542 |
| HLA-DMB | −0.011550 |
| FCGR2A | −0.011552 |
| MS4A7 | −0.011554 |
| ZYX | −0.011558 |
| SCIMP | −0.011559 |
| CD37 | −0.011562 |
| TEPP | −0.011562 |
| FAM162B | −0.011564 |
| PTGER4 | −0.011566 |
| KRT79 | −0.011576 |
| CLIP4 | −0.011591 |
| TYRP1 | −0.011595 |
| C4orf19 | −0.011604 |
| ACSF2 | −0.011607 |
| LTBP2 | −0.011610 |
| ZMYND15 | −0.011613 |
| GUCA2A | −0.011613 |
| USP44 | −0.011615 |
| PLA1A | −0.011617 |
| UNC45B | −0.011619 |
| SOCS3 | −0.011621 |
| RILP | −0.011626 |
| LONRF3 | −0.011632 |
| LTBP4 | −0.011633 |
| PI3 | −0.011634 |
| LAIR1 | −0.011636 |
| CFAP126 | −0.011640 |
| PHLDA3 | −0.011650 |
| TSC22D3 | −0.011662 |
| AHR | −0.011664 |
| SLA | −0.011666 |
| KYNU | −0.011688 |
| MMP7 | −0.011697 |
| PODN | −0.011708 |
| ICAM5 | −0.011709 |
| SFRP2 | −0.011716 |
| PLLP | −0.011719 |
| GADD45B | −0.011722 |
| CNN1 | −0.011733 |
| NCKAP1L | −0.011735 |
| FAM3D | −0.011740 |
| PTGES | −0.011745 |
| ADGB | −0.011745 |
| PRSS12 | −0.011750 |
| LDB2 | −0.011763 |
| HEG1 | −0.011773 |
| RAB7B | −0.011774 |
| DNASE2B | −0.011776 |
| EMP3 | −0.011785 |
| MVP | −0.011796 |
| C1QC | −0.011808 |
| FOXF2 | −0.011810 |
| CLDN5 | −0.011814 |
| KLRB1 | −0.011827 |
| F10 | −0.011832 |
| IFITM3 | −0.011841 |
| IL2RG | −0.011848 |
| BMP2 | −0.011857 |
| CD33 | −0.011859 |
| WDFY4 | −0.011862 |
| CLEC4M | −0.011862 |
| ENG | −0.011863 |
| WWC2 | −0.011869 |
| UBXN10 | −0.011873 |
| C10orf128 | −0.011873 |
| IGFBP4 | −0.011878 |
| METTL7B | −0.011879 |
| HCK | −0.011882 |
| TMEM119 | −0.011884 |
| NCR3 | −0.011889 |
| GIMAP8 | −0.011891 |
| P2RY12 | −0.011893 |
| TNFRSF6B | −0.011898 |
| SPTLC3 | −0.011903 |
| CTF1 | −0.011907 |
| MMP19 | −0.011908 |
| IDO1 | −0.011912 |
| SEMA4A | −0.011915 |
| HPN | −0.011916 |
| SECTM1 | −0.011929 |
| SIRPB2 | −0.011937 |
| VGLL3 | −0.011941 |
| PROS1 | −0.011955 |
| PRICKLE1 | −0.011957 |
| GREM2 | −0.011962 |
| RBMS3 | −0.011966 |
| FAM83E | −0.011967 |
| CCR2 | −0.011968 |
| PTPRB | −0.011973 |
| AXL | −0.011977 |
| FCER1G | −0.011979 |
| CYSLTR1 | −0.011983 |
| PLA2G12B | −0.011983 |
| ANXA2 | −0.011990 |
| PDCD1LG2 | −0.011991 |
| ATP1A2 | −0.011996 |
| KLK5 | −0.011997 |
| APOBEC3A | −0.012015 |
| SLC15A3 | −0.012020 |
| ITGA3 | −0.012031 |
| CD300E | −0.012035 |
| SLC38A5 | −0.012040 |
| RNASE4 | −0.012052 |
| S100A11 | −0.012057 |
| NNMT | −0.012078 |
| TRPV2 | −0.012088 |
| PMP22 | −0.012090 |
| STAC | −0.012091 |
| XAGE1B | −0.012103 |
| SNX20 | −0.012107 |
| MROH9 | −0.012108 |
| CTSD | −0.012110 |
| TNS4 | −0.012115 |
| MFSD2A | −0.012119 |
| PTGIS | −0.012120 |
| PARM1 | −0.012126 |
| VIPR1 | −0.012129 |
| FHL1 | −0.012138 |
| AHCYL2 | −0.012140 |
| CD53 | −0.012150 |
| SLC47A1 | −0.012156 |
| PECAM1 | −0.012161 |
| SIK1 | −0.012165 |
| SLC1A7 | −0.012175 |
| CYP2F1 | −0.012182 |
| TMEM37 | −0.012190 |
| CLIC2 | −0.012200 |
| RHOBTB2 | −0.012210 |
| MYADM | −0.012246 |
| CLCA2 | −0.012249 |
| PRKCQ | −0.012254 |
| BLVRB | −0.012275 |
| ODF3L1 | −0.012277 |
| SLC1A1 | −0.012281 |
| RSP03 | −0.012290 |
| CISH | −0.012293 |
| CPED1 | −0.012298 |
| TP63 | −0.012308 |
| LMOD1 | −0.012309 |
| ORM2 | −0.012321 |
| MACC1 | −0.012327 |
| RBP2 | −0.012337 |
| MUSTN1 | −0.012340 |
| NR3C2 | −0.012349 |
| SLCO2B1 | −0.012353 |
| C1orf158 | −0.012354 |
| GPRIN2 | −0.012360 |
| SH3GL3 | −0.012363 |
| CARD16 | −0.012363 |
| PTCRA | −0.012371 |
| C6orf223 | −0.012376 |
| CCDC60 | −0.012378 |
| CYP1A2 | −0.012389 |
| PAQR5 | −0.012403 |
| CYP2C18 | −0.012413 |
| GATA6 | −0.012425 |
| CELSR1 | −0.012430 |
| LGALS2 | −0.012431 |
| HSPB7 | −0.012432 |
| CD40LG | −0.012436 |
| ANOS1 | −0.012443 |
| CST7 | −0.012449 |
| C16orf54 | −0.012451 |
| GRAMD2 | −0.012451 |
| GDF5 | −0.012453 |
| CDH16 | −0.012454 |
| LMO7 | −0.012455 |
| FGD5 | −0.012471 |
| SERPINF2 | −0.012475 |
| MAOB | −0.012485 |
| ITM2A | −0.012488 |
| SLC27A6 | −0.012495 |
| C2orf40 | −0.012495 |
| SLC13A2 | −0.012498 |
| SLC52A3 | −0.012514 |
| LGALS12 | −0.012528 |
| UNC13D | −0.012532 |
| KCNJ5 | −0.012535 |
| FFAR4 | −0.012537 |
| NACA2 | −0.012549 |
| XCR1 | −0.012554 |
| SQRDL | −0.012555 |
| OVCH1 | −0.012555 |
| RARRES3 | −0.012565 |
| TMEM265 | −0.012567 |
| SEMA3G | −0.012570 |
| TMBIM1 | −0.012570 |
| HPSE2 | −0.012575 |
| PLAC9 | −0.012583 |
| PDZD2 | −0.012605 |
| SERPINB1 | −0.012622 |
| FILIP1 | −0.012633 |
| HLA-E | −0.012642 |
| CARD6 | −0.012661 |
| FGF7 | −0.012664 |
| MYOF | −0.012683 |
| RASSF9 | −0.012687 |
| MAL | −0.012690 |
| TPSD1 | −0.012691 |
| CD22 | −0.012697 |
| FXYD3 | −0.012700 |
| BTK | −0.012702 |
| ABCA3 | −0.012714 |
| CHRM1 | −0.012714 |
| HMGCLL1 | −0.012717 |
| SRD5A2 | −0.012727 |
| ADAMTSL4 | −0.012745 |
| UBTD1 | −0.012761 |
| VMO1 | −0.012774 |
| SMIM6 | −0.012789 |
| GCNT4 | −0.012794 |
| GLIPR2 | −0.012799 |
| ANGPTL1 | −0.012802 |
| VAMP8 | −0.012818 |
| PROSER2 | −0.012819 |
| VAMP5 | −0.012819 |
| SNURF | −0.012822 |
| COL6A6 | −0.012827 |
| KCNS1 | −0.012832 |
| BCL2A1 | −0.012836 |
| PDE4A | −0.012845 |
| PHYHD1 | −0.012851 |
| CAPG | −0.012865 |
| EGF | −0.012870 |
| NR4A3 | −0.012871 |
| CD163 | −0.012873 |
| CHI3L1 | −0.012874 |
| TRABD2A | −0.012876 |
| HDC | −0.012884 |
| PZP | −0.012889 |
| HLF | −0.012920 |
| C2orf91 | −0.012932 |
| CALCRL | −0.012944 |
| PSORS1C1 | −0.012947 |
| HSPB3 | −0.012952 |
| EVI2B | −0.012965 |
| CD5L | −0.012982 |
| SERPIND1 | −0.012985 |
| BTC | −0.012988 |
| DTHD1 | −0.012988 |
| SYNPO | −0.012989 |
| ANG | −0.012990 |
| ACVRL1 | −0.012998 |
| ALOX5 | −0.012999 |
| TMEM52B | −0.013018 |
| EMP2 | −0.013018 |
| CCL24 | −0.013022 |
| LAPTM5 | −0.013023 |
| S1PR1 | −0.013035 |
| RGS13 | −0.013044 |
| CSF2RA | −0.013058 |
| S100A12 | −0.013059 |
| ABO | −0.013062 |
| RAB43 | −0.013063 |
| EMCN | −0.013064 |
| SLC16A4 | −0.013065 |
| FAM46B | −0.013075 |
| NCMAP | −0.013076 |
| CDHR4 | −0.013099 |
| CASS4 | −0.013117 |
| PRF1 | −0.013121 |
| BTNL8 | −0.013128 |
| SRGN | −0.013130 |
| PIP | −0.013133 |
| RASGRP4 | −0.013147 |
| EDNRB | −0.013156 |
| PCDH15 | −0.013159 |
| CTGF | −0.013176 |
| AGTR1 | −0.013193 |
| CD44 | −0.013194 |
| C1QB | −0.013206 |
| RARRES2 | −0.013208 |
| TLR3 | −0.013209 |
| CDC42EP1 | −0.013209 |
| EFCC1 | −0.013213 |
| ACE2 | −0.013222 |
| COL4A4 | −0.013224 |
| RP1 | −0.013230 |
| PRAM1 | −0.013233 |
| GCSAML | −0.013236 |
| C1QA | −0.013240 |
| AQP7 | −0.013246 |
| SOD3 | −0.013250 |
| GATA3 | −0.013261 |
| HLA-G | −0.013271 |
| CEACAM21 | −0.013271 |
| FGFBP2 | −0.013274 |
| CXCL3 | −0.013277 |
| RSPO4 | −0.013288 |
| YAP1 | −0.013294 |
| HCST | −0.013302 |
| PON3 | −0.013312 |
| CCND1 | −0.013313 |
| TNFSF9 | −0.013318 |
| DUSP1 | −0.013323 |
| C1orf186 | −0.013338 |
| NXF3 | −0.013340 |
| CD4 | −0.013344 |
| FREM3 | −0.013346 |
| AJUBA | −0.013347 |
| CSRNP1 | −0.013363 |
| PCP4L1 | −0.013370 |
| ABCC3 | −0.013379 |
| APOC1 | −0.013382 |
| CATSPERD | −0.013384 |
| BCAS1 | −0.013387 |
| ITGAL | −0.013391 |
| DHRS9 | −0.013396 |
| CD300LF | −0.013405 |
| NPR3 | −0.013410 |
| IRX1 | −0.013430 |
| C1orf162 | −0.013430 |
| LST1 | −0.013442 |
| FLVCR2 | −0.013442 |
| SH2D4A | −0.013458 |
| LRP2 | −0.013469 |
| C8orf34 | −0.013469 |
| GATA5 | −0.013487 |
| SEC14L3 | −0.013495 |
| SRPX | −0.013496 |
| C1orf106 | −0.013516 |
| CEBPA | −0.013518 |
| TBC1D2 | −0.013519 |
| ITGB2 | −0.013523 |
| CYBB | −0.013526 |
| AMY1A | −0.013533 |
| AKR1C1 | −0.013534 |
| SVEP1 | −0.013535 |
| FOSL1 | −0.013540 |
| OSGIN1 | −0.013555 |
| ROBO4 | −0.013561 |
| OVCH2 | −0.013564 |
| SDC4 | −0.013565 |
| PRDM16 | −0.013575 |
| IL1R1 | −0.013578 |
| EMP1 | −0.013588 |
| CD36 | −0.013589 |
| SLC7A7 | −0.013601 |
| APOBR | −0.013622 |
| KRT27 | −0.013623 |
| SLC5A8 | −0.013624 |
| SCN1A | −0.013635 |
| GGTLC2 | −0.013656 |
| MMRN1 | −0.013662 |
| TBX5 | −0.013670 |
| OSMR | −0.013672 |
| MRGPRF | −0.013688 |
| SPI1 | −0.013712 |
| S100P | −0.013716 |
| MYL9 | −0.013726 |
| TMEM130 | −0.013730 |
| TMEM125 | −0.013732 |
| NPC2 | −0.013740 |
| EVPL | −0.013740 |
| HSD17B6 | −0.013742 |
| LDHD | −0.013764 |
| CCRL2 | −0.013768 |
| MT1M | −0.013768 |
| C8orf4 | −0.013773 |
| S100A3 | −0.013779 |
| ANPEP | −0.013786 |
| SERPING1 | −0.013801 |
| PRELP | −0.013801 |
| CTSS | −0.013804 |
| PTRF | −0.013804 |
| ANKRD66 | −0.013817 |
| AGBL1 | −0.013822 |
| CCR6 | −0.013827 |
| ADGRG6 | −0.013828 |
| SEMA3E | −0.013830 |
| SLIT3 | −0.013833 |
| GSTA3 | −0.013858 |
| EGR1 | −0.013865 |
| TMEM150B | −0.013872 |
| AOX1 | −0.013893 |
| PTPRQ | −0.013898 |
| SIGLEC14 | −0.013905 |
| C10orf107 | −0.013926 |
| S100A16 | −0.013926 |
| ERN2 | −0.013928 |
| CAMP | −0.013928 |
| C5AR1 | −0.013933 |
| IHH | −0.013933 |
| HBA1 | −0.013935 |
| S100A6 | −0.013937 |
| ANGPT4 | −0.013941 |
| GAPT | −0.013950 |
| TNS1 | −0.013960 |
| RRAS | −0.013962 |
| AGRP | −0.013962 |
| LTF | −0.013962 |
| CASQ2 | −0.013982 |
| AMY1B | −0.013989 |
| FGR | −0.013989 |
| LIMS2 | −0.013998 |
| CLU | −0.014001 |
| ITIH5 | −0.014003 |
| ACADL | −0.014012 |
| AVPI1 | −0.014016 |
| C1orf87 | −0.014023 |
| LRRC18 | −0.014031 |
| NLRP12 | −0.014032 |
| OASL | −0.014035 |
| PCOLCE2 | −0.014037 |
| TGM2 | −0.014057 |
| SLC26A9 | −0.014058 |
| CDIB | −0.014082 |
| RAC2 | −0.014095 |
| TLR2 | −0.014097 |
| ARHGAP31 | −0.014111 |
| XAGE1A | −0.014118 |
| KPNA7 | −0.014119 |
| SIGLEC5 | −0.014124 |
| CFI | −0.014129 |
| TNFSF13 | −0.014135 |
| CCL20 | −0.014138 |
| AHNAK | −0.014152 |
| CREB3L1 | −0.014159 |
| DOK2 | −0.014170 |
| TNIP3 | −0.014183 |
| MALRD1 | −0.014188 |
| TTC29 | −0.014195 |
| RAMP3 | −0.014199 |
| TLR8 | −0.014217 |
| TIMP3 | −0.014230 |
| CLEC12A | −0.014230 |
| NTN4 | −0.014244 |
| SYNDIG1L | −0.014249 |
| IL6R | −0.014276 |
| LILRA5 | −0.014282 |
| SULT1A2 | −0.014291 |
| ZFP36 | −0.014307 |
| PTAFR | −0.014316 |
| MPZL2 | −0.014319 |
| SULT2B1 | −0.014324 |
| TYROBP | −0.014333 |
| AKR1C2 | −0.014336 |
| DNAH9 | −0.014346 |
| MYH11 | −0.014346 |
| CCBE1 | −0.014404 |
| ACP5 | −0.014416 |
| SYT15 | −0.014433 |
| HBEGF | −0.014442 |
| SLCO2A1 | −0.014514 |
| SELENBP1 | −0.014538 |
| P2RY2 | −0.014545 |
| CYBRD1 | −0.014549 |
| LY86 | −0.014549 |
| IL1RN | −0.014550 |
| ANXA1 | −0.014553 |
| SHROOM4 | −0.014561 |
| SGCG | −0.014570 |
| HSPB8 | −0.014584 |
| NCF2 | −0.014617 |
| MT1A | −0.014634 |
| CD69 | −0.014636 |
| MAOA | −0.014655 |
| TMEM212 | −0.014669 |
| LILRA2 | −0.014672 |
| CATSPER1 | −0.014675 |
| F3 | −0.014689 |
| ITGAM | −0.014690 |
| TMEM190 | −0.014708 |
| TEK | −0.014710 |
| CSF2 | −0.014721 |
| TLCD2 | −0.014721 |
| SPOCK2 | −0.014729 |
| SERPINA3 | −0.014729 |
| ABCC6 | −0.014750 |
| ADGRF5 | −0.014755 |
| RGCC | −0.014766 |
| SLC11A1 | −0.014768 |
| SFTA3 | −0.014790 |
| TFPI2 | −0.014790 |
| CLEC4E | −0.014792 |
| B3GNT3 | −0.014796 |
| CFAP221 | −0.014802 |
| COL4A3 | −0.014803 |
| MATN3 | −0.014816 |
| SHE | −0.014828 |
| SULT1A4 | −0.014828 |
| GSTA2 | −0.014834 |
| ITGA8 | −0.014846 |
| HLA-DMA | −0.014862 |
| ODAM | −0.014870 |
| ST8SIA6 | −0.014871 |
| A2M | −0.014877 |
| PLA2G4A | −0.014887 |
| SIGLEC11 | −0.014916 |
| FOXF1 | −0.014919 |
| ACSL5 | −0.014931 |
| CHIT1 | −0.014947 |
| GPR39 | −0.014948 |
| NPR1 | −0.014960 |
| SHISA3 | −0.014966 |
| TFPI | −0.014968 |
| CYR61 | −0.014969 |
| TMEM139 | −0.015019 |
| CTSW | −0.015021 |
| FGFR2 | −0.015034 |
| PPARG | −0.015036 |
| EPHA2 | −0.015061 |
| HRCT1 | −0.015083 |
| CX3CL1 | −0.015087 |
| FCGR3B | −0.015092 |
| LDLRAD1 | −0.015104 |
| TGFBR2 | −0.015107 |
| CRTAC1 | −0.015136 |
| MNDA | −0.015138 |
| TNNI2 | −0.015138 |
| CX3CR1 | −0.015138 |
| ALOX15 | −0.015139 |
| DKK1 | −0.015170 |
| THBD | −0.015178 |
| CSF3R | −0.015218 |
| DCSTAMP | −0.015220 |
| DCN | −0.015230 |
| KRT13 | −0.015264 |
| CA4 | −0.015265 |
| GYPE | −0.015291 |
| MYH2 | −0.015319 |
| PAPSS2 | −0.015329 |
| SELPLG | −0.015331 |
| PRSS21 | −0.015360 |
| C2orf71 | −0.015365 |
| ABCA8 | −0.015365 |
| HBA2 | −0.015366 |
| DPEP2 | −0.015368 |
| NEDD9 | −0.015375 |
| CLEC10A | −0.015384 |
| SYNC | −0.015399 |
| C2 | −0.015413 |
| CCDC69 | −0.015415 |
| ORM1 | −0.015450 |
| CSTA | −0.015470 |
| MYRF | −0.015476 |
| CD1A | −0.015494 |
| PLBD1 | −0.015532 |
| FIBIN | −0.015533 |
| DLC1 | −0.015534 |
| CLDN2 | −0.015544 |
| EPAS1 | −0.015576 |
| ABI3BP | −0.015577 |
| HLA-DPB1 | −0.015588 |
| SYT8 | −0.015594 |
| S1PR4 | −0.015630 |
| TREM2 | −0.015641 |
| EREG | −0.015652 |
| ERP27 | −0.015669 |
| PGM5 | −0.015672 |
| CNN2 | −0.015692 |
| IL6 | −0.015712 |
| S100A8 | −0.015720 |
| PRODH | −0.015775 |
| HLA-DQA2 | −0.015779 |
| ALDH2 | −0.015779 |
| FAM189A2 | −0.015781 |
| LGALS3 | −0.015781 |
| IRX6 | −0.015782 |
| HLA-DPA1 | −0.015784 |
| CFD | −0.015788 |
| SLC51B | −0.015789 |
| ILIA | −0.015822 |
| RAB27B | −0.015843 |
| ADRB2 | −0.015848 |
| RBP4 | −0.015849 |
| IL33 | −0.015862 |
| FGF10 | −0.015888 |
| FAM81B | −0.015898 |
| CST5 | −0.015922 |
| ADCY8 | −0.015925 |
| CXCL16 | −0.015930 |
| GJA5 | −0.015951 |
| HBB | −0.015965 |
| MSR1 | −0.015978 |
| JCHAIN | −0.015985 |
| GGT6 | −0.015994 |
| CCL18 | −0.016010 |
| HLA-DQB1 | −0.016015 |
| NFE2 | −0.016025 |
| ANGPT1 | −0.016041 |
| RNF128 | −0.016064 |
| FPR1 | −0.016087 |
| ADRB1 | −0.016091 |
| C1QTNF7 | −0.016095 |
| FGFR4 | −0.016127 |
| STOML3 | −0.016201 |
| PLA2G10 | −0.016203 |
| HLA-DOA | −0.016206 |
| CCDC68 | −0.016220 |
| ACKR1 | −0.016226 |
| FOXQ1 | −0.016233 |
| CCL13 | −0.016253 |
| HCAR3 | −0.016261 |
| COLEC12 | −0.016264 |
| CD300C | −0.016272 |
| SCNN1B | −0.016302 |
| ADIRE | −0.016310 |
| CCDC170 | −0.016332 |
| ADIRP | −0.016334 |
| HLA-DQA1 | −0.016339 |
| C2orf54 | −0.016346 |
| CXCR1 | −0.016350 |
| ADGRE5 | −0.016360 |
| HSPB6 | −0.016411 |
| HP | −0.016414 |
| WFDC12 | −0.016428 |
| ELF5 | −0.016456 |
| MYZAP | −0.016540 |
| TEKT1 | −0.016543 |
| SGMS2 | −0.016549 |
| CH25H | −0.016552 |
| HLA-DRA | −0.016601 |
| SELP | −0.016609 |
| STX11 | −0.016626 |
| MAMDC2 | −0.016640 |
| ATOH8 | −0.016642 |
| FBP1 | −0.016669 |
| LYZ | −0.016687 |
| ADGRF1 | −0.016700 |
| LYVE1 | −0.016734 |
| S100A9 | −0.016754 |
| DKK2 | −0.016758 |
| WNT2 | −0.016770 |
| SPN | −0.016782 |
| SLC16A5 | −0.016785 |
| B3GNT7 | −0.016822 |
| CD68 | −0.016872 |
| PRX | −0.016925 |
| CPM | −0.016927 |
| SIGLEC6 | −0.016962 |
| ADH1C | −0.016969 |
| CD1E | −0.016972 |
| DUOX1 | −0.016986 |
| EFEMP1 | −0.017000 |
| CTSG | −0.017006 |
| EVPLL | −0.017036 |
| C6 | −0.017049 |
| ITLN1 | −0.017050 |
| HLA-DRB1 | −0.017058 |
| C11orf88 | −0.017069 |
| LRG1 | −0.017071 |
| TMEM213 | −0.017072 |
| PI16 | −0.017077 |
| PRSS8 | −0.017085 |
| AKR1C3 | −0.017116 |
| CAPSL | −0.017128 |
| EHD2 | −0.017157 |
| PRG4 | −0.017161 |
| BMP5 | −0.017207 |
| ANKRD29 | −0.017213 |
| OGN | −0.017222 |
| AOC3 | −0.017232 |
| GPD1 | −0.017233 |
| TNXB | −0.017236 |
| GPX3 | −0.017251 |
| HKDC1 | −0.017251 |
| ALDH3A1 | −0.017290 |
| CD74 | −0.017314 |
| LAMC2 | −0.017377 |
| GSTM5 | −0.017390 |
| DRAM1 | −0.017406 |
| CD55 | −0.017413 |
| NINJ2 | −0.017424 |
| HRASLS5 | −0.017443 |
| S100A10 | −0.017458 |
| KNDC1 | −0.017464 |
| SNTN | −0.017477 |
| WDR38 | −0.017483 |
| HYAL1 | −0.017485 |
| PDPN | −0.017496 |
| AGR2 | −0.017498 |
| FBLN5 | −0.017535 |
| AQP9 | −0.017540 |
| DPT | −0.017561 |
| NTF4 | −0.017601 |
| EDN1 | −0.017638 |
| MUC5B | −0.017685 |
| FGA | −0.017698 |
| CPAMD8 | −0.017709 |
| SEC14L4 | −0.017780 |
| PPBP | −0.017789 |
| ADGRE1 | −0.017848 |
| S100A4 | −0.017855 |
| ADGRE3 | −0.017937 |
| HPGDS | −0.017937 |
| ALPL | −0.017943 |
| ADGRD1 | −0.017944 |
| IYD | −0.017945 |
| APOC2 | −0.017947 |
| CXCR2 | −0.017956 |
| OSCAR | −0.017960 |
| C7 | −0.017977 |
| SCN2B | −0.018037 |
| SHH | −0.018037 |
| SIRPB1 | −0.018058 |
| C1orf167 | −0.018098 |
| KLK7 | −0.018119 |
| CYS1 | −0.018145 |
| TUBB3 | −0.018181 |
| ITGB4 | −0.018226 |
| MAP1LC3C | −0.018255 |
| KLK10 | −0.018290 |
| BMP3 | −0.018298 |
| CCL14 | −0.018313 |
| CYP2S1 | −0.018375 |
| P3H2 | −0.018382 |
| CHRDL1 | −0.018388 |
| CAPN8 | −0.018437 |
| AADAC | −0.018447 |
| HHLA2 | −0.018498 |
| CXCL2 | −0.018506 |
| CTSH | −0.018528 |
| DUOXA1 | −0.018529 |
| SGCA | −0.018533 |
| FLRT3 | −0.018574 |
| RSI | −0.018588 |
| RSPO1 | −0.018610 |
| FCN1 | −0.018617 |
| SLC39A8 | −0.018665 |
| C1orf116 | −0.018713 |
| PTGS2 | −0.018732 |
| HCAR2 | −0.018748 |
| RNASE1 | −0.018771 |
| ALPPL2 | −0.018779 |
| CD1C | −0.018780 |
| VSIG2 | −0.018792 |
| VSIG4 | −0.018795 |
| HLA-DQB2 | −0.018804 |
| AQP1 | −0.018856 |
| IL7R | −0.018912 |
| CD52 | −0.018928 |
| SCN7A | −0.018972 |
| KRT7 | −0.019005 |
| RSPO2 | −0.019020 |
| FMO2 | −0.019028 |
| C20orf85 | −0.019046 |
| MUC1 | −0.019101 |
| FXYD1 | −0.019213 |
| RRAD | −0.019241 |
| ICAM1 | −0.019256 |
| PCSK9 | −0.019290 |
| MYOC | −0.019323 |
| ALDH3B1 | −0.019325 |
| TACSTD2 | −0.019333 |
| FPR2 | −0.019339 |
| MFAP4 | −0.019360 |
| CHIA | −0.019366 |
| ARSE | −0.019412 |
| TMEM92 | −0.019416 |
| CCL21 | −0.019437 |
| LIPH | −0.019455 |
| SLC22A3 | −0.019466 |
| FAM216B | −0.019483 |
| GPIHBP1 | −0.019542 |
| CCL22 | −0.019553 |
| WISP2 | −0.019587 |
| LCN2 | −0.019592 |
| FAM107A | −0.019607 |
| GPM6A | −0.019613 |
| CLEC3B | −0.019670 |
| GSTA1 | −0.019745 |
| DPP4 | −0.019878 |
| TREM1 | −0.019900 |
| XAGE2 | −0.019902 |
| FOLR3 | −0.019904 |
| LAMP3 | −0.019949 |
| VGLL1 | −0.019975 |
| SH3RF2 | −0.019994 |
| CAV2 | −0.020013 |
| WNT3A | −0.020016 |
| CSF3 | −0.020038 |
| MALL | −0.020045 |
| MRC1 | −0.020063 |
| CCL17 | −0.020065 |
| FOSB | −0.020112 |
| ZBED2 | −0.020165 |
| TMEM100 | −0.020247 |
| HOPX | −0.020316 |
| CXCL5 | −0.020457 |
| PLA2G4F | −0.020487 |
| COL6A5 | −0.020506 |
| C3 | −0.020551 |
| ANXA3 | −0.020557 |
| ALOX5AP | −0.020596 |
| SDPR | −0.020600 |
| APOH | −0.020622 |
| ST6GALNAC1 | −0.020708 |
| STEAP4 | −0.020716 |
| F11 | −0.020907 |
| CCL23 | −0.020920 |
| MUC15 | −0.021057 |
| SERPINA1 | −0.021091 |
| GALNT5 | −0.021097 |
| CLIC5 | −0.021171 |
| LRRK2 | −0.021177 |
| IL1RL1 | −0.021193 |
| SCTR | −0.021220 |
| HPGD | −0.021259 |
| DAW1 | −0.021279 |
| INMT | −0.021287 |
| DES | −0.021311 |
| PTGDS | −0.021317 |
| SLC19A3 | −0.021412 |
| CFTR | −0.021445 |
| LRRN4 | −0.021477 |
| ITLN2 | −0.021478 |
| FCER1A | −0.021492 |
| VEPH1 | −0.021507 |
| CNTN6 | −0.021551 |
| HLA-DRB5 | −0.021553 |
| MMP28 | −0.021581 |
| ADAMTS8 | −0.021583 |
| CHI3L2 | −0.021649 |
| ANXA8L1 | −0.021694 |
| PDZK1IP1 | −0.021825 |
| ATPI3A4 | −0.021902 |
| AGR3 | −0.021930 |
| CAV1 | −0.021931 |
| MS4A2 | −0.021967 |
| TMPRSS2 | −0.022039 |
| HNF1B | −0.022090 |
| FCN3 | −0.022133 |
| AREG | −0.022186 |
| SOSTDC1 | −0.022189 |
| AQP5 | −0.022201 |
| LAMB3 | −0.022222 |
| SECI4L6 | −0.022237 |
| FGFBP1 | −0.022319 |
| HHIP | −0.022383 |
| ITGB6 | −0.022413 |
| ALOX15B | −0.022430 |
| GPA33 | −0.022447 |
| ANXA8 | −0.022470 |
| KCNJ15 | −0.022524 |
| C8B | −0.022649 |
| CD300LG | −0.022741 |
| TBX4 | −0.022883 |
| SLC6A4 | −0.022884 |
| SUSD2 | −0.023013 |
| ADH1B | −0.023061 |
| FABP4 | −0.023078 |
| AQP3 | −0.023172 |
| SCARA5 | −0.023264 |
| SLC22A31 | −0.023313 |
| FGG | −0.023493 |
| CPB2 | −0.023507 |
| RASGRF1 | −0.023524 |
| ANKRD1 | −0.023574 |
| GDF10 | −0.023584 |
| WNT7A | −0.023672 |
| S100A14 | −0.023674 |
| ICAM4 | −0.023711 |
| CLIC3 | −0.023723 |
| DPCR1 | −0.023820 |
| RETN | −0.023868 |
| CST6 | −0.023877 |
| OLR1 | −0.023965 |
| AGER | −0.024176 |
| TCF21 | −0.024208 |
| ALPP | −0.024401 |
| MS4A15 | −0.024495 |
| CLDN18 | −0.024795 |
| SLC46A2 | −0.025004 |
| BPIFB1 | −0.025089 |
| SDR16C5 | −0.025341 |
| TNNC1 | −0.025531 |
| CPA3 | −0.025846 |
| NOTCH2NL | −0.025885 |
| C19orf33 | −0.025997 |
| PLA2G1B | −0.026059 |
| SLPI | −0.026184 |
| CES1 | −0.026311 |
| NDNF | −0.026596 |
| CXCL17 | −0.026697 |
| UPK3B | −0.026718 |
| CEACAM6 | −0.026889 |
| KRT4 | −0.026975 |
| GKN2 | −0.027021 |
| CTSE | −0.027329 |
| AMY1C | −0.027356 |
| SLC6A14 | −0.027418 |
| TPSAB1 | −0.027421 |
| AGTR2 | −0.027492 |
| WIF1 | −0.027701 |
| TPSB2 | −0.027882 |
| LGI3 | −0.028047 |
| FOLR1 | −0.028416 |
| ROS1 | −0.028783 |
| MUC21 | −0.028905 |
| MARCO | −0.029014 |
| AQP4 | −0.029183 |
| C16orf89 | −0.029663 |
| PIGR | −0.029974 |
| GPRC5A | −0.030127 |
| MCEMP1 | −0.030373 |
| CYP4B1 | −0.030385 |
| PEBP4 | −0.030870 |
| DMBT1 | −0.030938 |
| C4BPA | −0.031076 |
| GGTLC1 | −0.031540 |
| SCGB3A1 | −0.032282 |
| NAPSA | −0.032301 |
| SCGB3A2 | −0.032459 |
| SFTPD | −0.032991 |
| SCEL | −0.033338 |
| SCGB1A1 | −0.033482 |
| MSLN | −0.034377 |
| PCDH20 | −0.034771 |
| SLC34A2 | −0.036437 |
| PGC | −0.036730 |
| SFTPB | −0.037152 |
| SFTA2 | −0.037941 |
| NDUFA7 | −0.038592 |
| SFTPA2 | −0.041274 |
| SFTPA1 | −0.044867 |
| SFTPC | −0.049137 |

TABLE 2
SCN gene signature weights, top 100 and bottom 100 genes

| | SCN |
|---|---|
| | signature |
| Gene | weight |
| TARDBP | 0.050046 |
| PIK3R3 | 0.049087 |
| DPYSL5 | 0.044226 |
| TMBIM4 | 0.042018 |
| PIK3R2 | 0.041165 |
| INSM1 | 0.040721 |
| TIMM10B | 0.039219 |
| KIF1A | 0.038317 |
| PSMA1 | 0.037698 |
| STXBP5L | 0.037609 |
| CHGA | 0.036387 |
| PAPD7 | 0.036124 |
| ELAVL3 | 0.035688 |
| DLL3 | 0.034705 |
| ASCL1 | 0.034372 |
| PI4K2B | 0.034356 |
| ZIC5 | 0.034346 |
| DLK1 | 0.034262 |
| SEZ6 | 0.034028 |
| FOXG1 | 0.033796 |
| GNG4 | 0.033750 |
| ATCAY | 0.033655 |
| ZIC2 | 0.033534 |
| RAB3B | 0.033273 |
| RIMS2 | 0.033236 |
| ACTL6B | 0.033103 |
| ZNF286A | 0.033099 |
| ONECUT2 | 0.032991 |
| INA | 0.032957 |
| NRSN1 | 0.032855 |
| PAGR1 | 0.032600 |
| CPLX2 | 0.032351 |
| BEST3 | 0.032243 |
| CELF3 | 0.032012 |
| DCX | 0.031961 |
| ST18 | 0.031529 |
| CACNA1A | 0.031512 |
| XKR7 | 0.031507 |
| LHX2 | 0.031425 |
| CACNA1B | 0.031268 |
| SYT4 | 0.031045 |
| C17orf100 | 0.031021 |
| PCSK1 | 0.030989 |
| TAGLN3 | 0.030809 |
| SOX11 | 0.030603 |
| FAM57B | 0.030310 |
| CHRNB2 | 0.030170 |
| NKX2-2 | 0.030022 |
| KCNC1 | 0.029939 |
| ISL1 | 0.029906 |
| HEPACAM2 | 0.029840 |
| ST8SIA3 | 0.029799 |
| DLX6 | 0.029756 |
| SCG3 | 0.029402 |
| PHF21B | 0.029396 |
| DLX1 | 0.029377 |
| ACSL6 | 0.029205 |
| SPATA13 | 0.029188 |
| NOL4 | 0.029159 |
| SEZ6L | 0.029159 |
| CDK5R2 | 0.029034 |
| CAMK2B | 0.028906 |
| B4GALNT4 | 0.028815 |
| TMEM179 | 0.028799 |
| CHGB | 0.028725 |
| RGS5 | 0.028715 |
| KCNH5 | 0.028658 |
| KIF5C | 0.028640 |
| MAST1 | 0.028625 |
| TMEM151B | 0.028579 |
| ZBTB8B | 0.028501 |
| MYT1 | 0.028396 |
| VGF | 0.028366 |
| RALYL | 0.028332 |
| SLC7A14 | 0.028238 |
| SYT13 | 0.028114 |
| UNC13A | 0.027888 |
| ST8SIA5 | 0.027832 |
| CAMK2N2 | 0.027811 |
| SCGN | 0.027799 |
| MPPED1 | 0.027698 |
| UNC80 | 0.027571 |
| NRXN1 | 0.027371 |
| HRH3 | 0.027336 |
| ELAVL4 | 0.027325 |
| CNTNAP2 | 0.027288 |
| SYT14 | 0.027228 |
| OGDHL | 0.027225 |
| ZMAT4 | 0.027169 |
| TCERG1L | 0.026964 |
| ZDHHC22 | 0.026950 |
| GRM4 | 0.026729 |
| AMH | 0.026692 |
| SRRM4 | 0.026690 |
| DGKB | 0.026580 |
| FOXN4 | 0.026556 |
| NEUROD1 | 0.026519 |
| PCDH8 | 0.026511 |
| RIMBP2 | 0.026498 |
| SULT4A1 | 0.026466 |
| SFTPC | −0.049137 |
| SFTPA1 | −0.044867 |
| SFTPA2 | −0.041274 |
| NDUFA7 | −0.038592 |
| SFTA2 | −0.037941 |
| SFTPB | −0.037152 |
| PGC | −0.036730 |
| SLC34A2 | −0.036437 |
| PCDH20 | −0.034771 |
| MSLN | −0.034377 |
| SCGB1A1 | −0.033482 |
| SCEL | −0.033338 |
| SFTPD | −0.032991 |
| SCGB3A2 | −0.032459 |
| NAPSA | −0.032301 |
| SCGB3A1 | −0.032282 |
| GGTLC1 | −0.031540 |
| C4BPA | −0.031076 |
| DMBT1 | −0.030938 |
| PEBP4 | −0.030870 |
| CYP4B1 | −0.030385 |
| MCEMP1 | −0.030373 |
| GPRC5A | −0.030127 |
| PIGR | −0.029974 |
| C16orf89 | −0.029663 |
| AQP4 | −0.029183 |
| MARCO | −0.029014 |
| MUC21 | −0.028905 |
| ROS1 | −0.028783 |
| FOLR1 | −0.028416 |
| LGI3 | −0.028047 |
| TPSB2 | −0.027882 |
| WIF1 | −0.027701 |
| AGTR2 | −0.027492 |
| TPSAB1 | −0.027421 |
| SLC6A14 | −0.027418 |
| AMY1C | −0.027356 |
| CTSE | −0.027329 |
| GKN2 | −0.027021 |
| KRT4 | −0.026975 |
| CEACAM6 | −0.026889 |
| UPK3B | −0.026718 |
| CXCL17 | −0.026697 |
| NDNF | −0.026596 |
| CES1 | −0.026311 |
| SLPI | −0.026184 |
| PLA2G1B | −0.026059 |
| C19orf33 | −0.025997 |
| NOTCH2NL | −0.025885 |
| CPA3 | −0.025846 |
| TNNC1 | −0.025531 |
| SDR16C5 | −0.025341 |
| BPIFB1 | −0.025089 |
| SLC46A2 | −0.025004 |
| CLDN18 | −0.024795 |
| MS4A15 | −0.024495 |
| ALPP | −0.024401 |
| TCF21 | −0.024208 |
| AGER | −0.024176 |
| OLR1 | −0.023965 |
| CST6 | −0.023877 |
| RETN | −0.023868 |
| DPCR1 | −0.023820 |
| CLIC3 | −0.023723 |
| ICAM4 | −0.023711 |
| S100A14 | −0.023674 |
| WNT7A | −0.023672 |
| GDF10 | −0.023584 |
| ANKRD1 | −0.023574 |
| RASGRF1 | −0.023524 |
| CPB2 | −0.023507 |
| FGG | −0.023493 |
| SLC22A31 | −0.023313 |
| SCARA5 | −0.023264 |
| AQP3 | −0.023172 |
| FABP4 | −0.023078 |
| ADH1B | −0.023061 |
| SUSD2 | −0.023013 |
| SLC6A4 | −0.022884 |
| TBX4 | −0.022883 |
| CD300LG | −0.022741 |
| C8B | −0.022649 |
| KCNJ15 | −0.022524 |
| ANXA8 | −0.022470 |
| GPA33 | −0.022447 |
| ALOX15B | −0.022430 |
| ITGB6 | −0.022413 |
| HHIP | −0.022383 |
| FGFBP1 | −0.022319 |
| SEC14L6 | −0.022237 |
| LAMB3 | −0.022222 |
| AQP5 | −0.022201 |
| SOSTDC1 | −0.022189 |
| AREG | −0.022186 |
| FCN3 | −0.022133 |
| HNF1B | −0.022090 |
| TMPRSS2 | −0.022039 |
| MS4A2 | −0.021967 |
| CAV1 | −0.021931 |
| AGR3 | −0.021930 |

TABLE 3
SCN gene signature weights, top 500 and bottom 500 genes

| | SCN |
|---|---|
| | signature |
| Gene | weight |
| TARDBP | 0.050046 |
| PIK3R3 | 0.049087 |
| DPYSL5 | 0.044226 |
| TMBEM4 | 0.042018 |
| PIK3R2 | 0.041165 |
| INSM1 | 0.040721 |
| TIMM10B | 0.039219 |
| KIF1A | 0.038317 |
| PSMA1 | 0.037698 |
| STXBP5L | 0.037609 |
| CHGA | 0.036387 |
| PAPD7 | 0.036124 |
| ELAVL3 | 0.035688 |
| DLL3 | 0.034705 |
| ASCL1 | 0.034372 |
| PI4K2B | 0.034356 |
| ZIC5 | 0.034346 |
| DLK1 | 0.034262 |
| SEZ6 | 0.034028 |
| FOXG1 | 0.033796 |
| GNG4 | 0.033750 |
| ATCAY | 0.033655 |
| ZIC2 | 0.033534 |
| RAB3B | 0.033273 |
| RIMS2 | 0.033236 |
| ACTL6B | 0.033103 |
| ZNF286A | 0.033099 |
| ONECUT2 | 0.032991 |
| INA | 0.032957 |
| NRSN1 | 0.032855 |
| PAGR1 | 0.032600 |
| CPLX2 | 0.032351 |
| BEST3 | 0.032243 |
| CELF3 | 0.032012 |
| DCX | 0.031961 |
| ST18 | 0.031529 |
| CACNA1A | 0.031512 |
| XKR7 | 0.031507 |
| LHX2 | 0.031425 |
| CACNA1B | 0.031268 |
| SYT4 | 0.031045 |
| C17orf100 | 0.031021 |
| PCSK1 | 0.030989 |
| TAGLN3 | 0.030809 |
| SOX11 | 0.030603 |
| FAM57B | 0.030310 |
| CHRNB2 | 0.030170 |
| NKX2-2 | 0.030022 |
| KCNC1 | 0.029939 |
| ISL1 | 0.029906 |
| HEPACAM2 | 0.029840 |
| ST8SIA3 | 0.029799 |
| DLX6 | 0.029756 |
| SCG3 | 0.029402 |
| PHF21B | 0.029396 |
| DLX1 | 0.029377 |
| ACSL6 | 0.029205 |
| SPATA13 | 0.029188 |
| NOL4 | 0.029159 |
| SEZ6L | 0.029159 |
| CDK5R2 | 0.029034 |
| CAMK2B | 0.028906 |
| B4GALNT4 | 0.028815 |
| TMEM179 | 0.028799 |
| CHGB | 0.028725 |
| RGS5 | 0.028715 |
| KCNH5 | 0.028658 |
| KIF5C | 0.028640 |
| MAST1 | 0.028625 |
| TMEM151B | 0.028579 |
| ZBTB8B | 0.028501 |
| MYT1 | 0.028396 |
| VGF | 0.028366 |
| RALYL | 0.028332 |
| SLC7A14 | 0.028238 |
| SYT13 | 0.028114 |
| UNC13A | 0.027888 |
| ST8SIA5 | 0.027832 |
| CAMK2N2 | 0.027811 |
| SCGN | 0.027799 |
| MPPED1 | 0.027698 |
| UNC80 | 0.027571 |
| NRXN1 | 0.027371 |
| HRH3 | 0.027336 |
| ELAVL4 | 0.027325 |
| CNTNAP2 | 0.027288 |
| SYT14 | 0.027228 |
| OGDHL | 0.027225 |
| ZMAT4 | 0.027169 |
| TCERG1L | 0.026964 |
| ZDHHC22 | 0.026950 |
| GRM4 | 0.026729 |
| AMH | 0.026692 |
| SRRM4 | 0.026690 |
| DGKB | 0.026580 |
| FOXN4 | 0.026556 |
| NEUROD1 | 0.026519 |
| PCDH8 | 0.026511 |
| RIMBP2 | 0.026498 |
| SULT4A1 | 0.026466 |
| AMER3 | 0.026430 |
| TMEFF1 | 0.026347 |
| NKAIN1 | 0.026332 |
| BTBD17 | 0.026300 |
| SCN3A | 0.026154 |
| SVOP | 0.026100 |
| POU4F1 | 0.026037 |
| HOXD13 | 0.025985 |
| CBLN2 | 0.025911 |
| TRPM8 | 0.025897 |
| RUNDC3A | 0.025835 |
| IDS | 0.025572 |
| GRIK3 | 0.025522 |
| FSD1 | 0.025345 |
| HOXB9 | 0.025242 |
| HOXD11 | 0.025228 |
| BEX1 | 0.025117 |
| SLC6A17 | 0.025088 |
| CLN3 | 0.024931 |
| CRYBA2 | 0.024927 |
| NGB | 0.024816 |
| KCNH7 | 0.024805 |
| KIF5A | 0.024804 |
| 43535 | 0.024730 |
| COL2A1 | 0.024673 |
| TUBB2B | 0.024657 |
| UNC79 | 0.024609 |
| BARX1 | 0.024534 |
| TRIM9 | 0.024446 |
| POU3F2 | 0.024437 |
| KCNB2 | 0.024396 |
| KCNK9 | 0.024392 |
| DRD2 | 0.024382 |
| SYT5 | 0.024358 |
| KCNA1 | 0.024348 |
| C12orf56 | 0.024274 |
| PRAME | 0.024245 |
| SOX1 | 0.024178 |
| PEX5L | 0.024159 |
| CAMKV | 0.024143 |
| SALL1 | 0.024140 |
| CABP7 | 0.024106 |
| BRSK2 | 0.024054 |
| PHYHIPL | 0.024008 |
| DLX2 | 0.023887 |
| PTPRN | 0.023751 |
| GAD1 | 0.023747 |
| C11orf71 | 0.023739 |
| FUT9 | 0.023668 |
| KCNH8 | 0.023608 |
| ELOVL2 | 0.023538 |
| GPC2 | 0.023503 |
| EPHA8 | 0.023369 |
| COL22A1 | 0.023360 |
| NXPH4 | 0.023348 |
| KCNH6 | 0.023265 |
| NKAIN2 | 0.023157 |
| GPR6 | 0.023028 |
| HES6 | 0.023018 |
| DLX5 | 0.022900 |
| AP3B2 | 0.022896 |
| CELSR3 | 0.022882 |
| TERT | 0.022839 |
| SNAP91 | 0.022802 |
| SALL3 | 0.022753 |
| VWA5B2 | 0.022720 |
| ECSCR | 0.022664 |
| E2F7 | 0.022589 |
| RTBDN | 0.022574 |
| ZACN | 0.022539 |
| GAD2 | 0.022517 |
| TPH1 | 0.022478 |
| CACNA1E | 0.022469 |
| CALML3 | 0.022441 |
| CNTNAP5 | 0.022435 |
| NEUROD4 | 0.022395 |
| 43528 | 0.022364 |
| CADPS | 0.022363 |
| GRIA2 | 0.022350 |
| HOXD10 | 0.022343 |
| TMEM145 | 0.022342 |
| SCN8A | 0.022120 |
| KIF18B | 0.022106 |
| ASPM | 0.022076 |
| PPM1E | 0.022048 |
| MRPS17 | 0.022007 |
| C1QL1 | 0.021997 |
| HMX2 | 0.021955 |
| KCNJ6 | 0.021950 |
| FBXO43 | 0.021870 |
| RIPPLY3 | 0.021866 |
| DEPDC1 | 0.021850 |
| SLC6A5 | 0.021804 |
| MYT1L | 0.021779 |
| NELL1 | 0.021770 |
| CTNNA2 | 0.021751 |
| CCNE2 | 0.021716 |
| SHOX2 | 0.021672 |
| GRP | 0.021669 |
| RNF183 | 0.021577 |
| TROAP | 0.021568 |
| CHD5 | 0.021561 |
| INSRR | 0.021543 |
| SLC35D3 | 0.021542 |
| GDAP1L1 | 0.021492 |
| SCGB1D2 | 0.021439 |
| TMEM178B | 0.021305 |
| CDH7 | 0.021291 |
| SIX3 | 0.021267 |
| CASKIN1 | 0.021263 |
| TRIM67 | 0.021229 |
| B3GALT5 | 0.021163 |
| SLCO5A1 | 0.021156 |
| HGFAC | 0.021152 |
| FBF1 | 0.021142 |
| EPHA7 | 0.021141 |
| ADCYAP1 | 0.021105 |
| NUF2 | 0.021039 |
| DIRAS2 | 0.021015 |
| SCN2A | 0.021014 |
| SNAP25 | 0.021013 |
| DPF1 | 0.020945 |
| UBE2QL1 | 0.020938 |
| RAD54L | 0.020914 |
| GP1BB | 0.020880 |
| CRABP1 | 0.020879 |
| CPLX3 | 0.020824 |
| NAT16 | 0.020801 |
| BRSK1 | 0.020795 |
| KIF14 | 0.020782 |
| HJURP | 0.020754 |
| KCNK12 | 0.020719 |
| ADAMTS18 | 0.020714 |
| MNX1 | 0.020709 |
| UNC5A | 0.020679 |
| COCH | 0.020621 |
| TOP2A | 0.020601 |
| ZNF695 | 0.020585 |
| FAM64A | 0.020571 |
| AMER2 | 0.020562 |
| NPPA | 0.020538 |
| CENPF | 0.020517 |
| EEF1A2 | 0.020507 |
| ELAVL2 | 0.020501 |
| DEPDC1B | 0.020444 |
| RIPPLY2 | 0.020407 |
| STMN2 | 0.020406 |
| TEX15 | 0.020386 |
| APC2 | 0.020370 |
| MSI1 | 0.020336 |
| NEURL1 | 0.020268 |
| DMRTA2 | 0.020162 |
| SHISA9 | 0.020137 |
| ABCC8 | 0.020127 |
| SCRT1 | 0.020095 |
| CCDC150 | 0.020087 |
| FAM83F | 0.020080 |
| KCNK10 | 0.020062 |
| HOXB8 | 0.020056 |
| ATOH7 | 0.020053 |
| TTK | 0.020048 |
| NEUROG2 | 0.020024 |
| TTBK1 | 0.019983 |
| STMN4 | 0.019952 |
| PDIA2 | 0.019920 |
| GLYATL1 | 0.019856 |
| POTEI | 0.019788 |
| DLGAP5 | 0.019781 |
| PBK | 0.019738 |
| DLGAP3 | 0.019721 |
| NXPH1 | 0.019719 |
| TMEM63C | 0.019718 |
| LRTM2 | 0.019709 |
| FAM111B | 0.019709 |
| HOXA10 | 0.019707 |
| FOXD1 | 0.019695 |
| CENPA | 0.019689 |
| DPYSL4 | 0.019676 |
| CNTN2 | 0.019641 |
| SHD | 0.019618 |
| HOXA13 | 0.019618 |
| FAM163B | 0.019603 |
| MEX3A | 0.019558 |
| OR51E2 | 0.019553 |
| CALCB | 0.019531 |
| SP8 | 0.019520 |
| WNK2 | 0.019514 |
| RAB26 | 0.019503 |
| PPFIA4 | 0.019480 |
| SPOCK1 | 0.019470 |
| RBFOX1 | 0.019457 |
| MATR3 | 0.019445 |
| MAGEA3 | 0.019440 |
| SAPCD2 | 0.019393 |
| ESPL1 | 0.019386 |
| HOXD4 | 0.019380 |
| SLC18A1 | 0.019365 |
| ATP1A3 | 0.019364 |
| GRM8 | 0.019360 |
| FOXD3 | 0.019355 |
| INHBE | 0.019344 |
| NDST4 | 0.019336 |
| BSN | 0.019310 |
| CER1 | 0.019298 |
| HIST1H2BH | 0.019294 |
| KIF4A | 0.019288 |
| GRM2 | 0.019262 |
| FRRS1L | 0.019244 |
| SOWAHA | 0.019237 |
| SCG2 | 0.019209 |
| ACRV1 | 0.019190 |
| KCNQ2 | 0.019163 |
| UHRF1 | 0.019149 |
| LCN15 | 0.019148 |
| RDM1 | 0.019132 |
| BUB1B | 0.019102 |
| CALB1 | 0.019033 |
| RGS17 | 0.019032 |
| CDC25C | 0.019021 |
| CLGN | 0.018981 |
| IQGAP3 | 0.018974 |
| LHFPL5 | 0.018970 |
| CLVS2 | 0.018965 |
| CNIH2 | 0.018937 |
| ATP2B3 | 0.018936 |
| CCDC177 | 0.018929 |
| UBE2C | 0.018927 |
| NCAM1 | 0.018922 |
| POLQ | 0.018917 |
| NEK2 | 0.018907 |
| TMSB15A | 0.018905 |
| NDC80 | 0.018901 |
| PCP4 | 0.018866 |
| MKRN3 | 0.018863 |
| MELK | 0.018847 |
| GTSE1 | 0.018820 |
| SKA1 | 0.018761 |
| EZH2 | 0.018753 |
| SLIT1 | 0.018740 |
| ST8SIA2 | 0.018740 |
| GABRQ | 0.018736 |
| CBS | 0.018664 |
| UCHL1 | 0.018653 |
| NMU | 0.018651 |
| LRRC7 | 0.018635 |
| MYBL2 | 0.018623 |
| DDC | 0.018576 |
| EXO1 | 0.018534 |
| FGF17 | 0.018531 |
| ARHGAP33 | 0.018529 |
| SIX2 | 0.018518 |
| SRSF12 | 0.018518 |
| TPX2 | 0.018506 |
| CDH2 | 0.018498 |
| NTN3 | 0.018492 |
| EMX1 | 0.018483 |
| NEIL3 | 0.018461 |
| OR51E1 | 0.018440 |
| NEUROG3 | 0.018429 |
| KCNK16 | 0.018396 |
| CKAP2L | 0.018388 |
| E2F2 | 0.018386 |
| KIF2C | 0.018371 |
| KIF15 | 0.018341 |
| ELOVL4 | 0.018340 |
| 43711 | 0.018318 |
| HIST1H2BG | 0.018297 |
| PROX1 | 0.018274 |
| L1CAM | 0.018270 |
| ACBD7 | 0.018253 |
| OPRD1 | 0.018246 |
| SRPK3 | 0.018213 |
| PAH | 0.018204 |
| RPRM | 0.018194 |
| SLC45A2 | 0.018157 |
| PIF1 | 0.018150 |
| GPRIN1 | 0.018138 |
| NCAPG | 0.018130 |
| CRHR1 | 0.018103 |
| SHISA8 | 0.018100 |
| MED12L | 0.018065 |
| CCKBR | 0.018033 |
| MTHFS | 0.018020 |
| DDX25 | 0.017979 |
| GHRH | 0.017948 |
| LRFN2 | 0.017940 |
| C19orf45 | 0.017932 |
| NCAN | 0.017921 |
| DSCAM | 0.017910 |
| MAGEA6 | 0.017900 |
| HPCA | 0.017899 |
| ASXL3 | 0.017861 |
| SPAG5 | 0.017821 |
| NUSAP1 | 0.017814 |
| C16orf59 | 0.017808 |
| RHBDL3 | 0.017805 |
| CDCA3 | 0.017797 |
| FAM155B | 0.017792 |
| BIRC5 | 0.017769 |
| RCOR2 | 0.017761 |
| CALCA | 0.017757 |
| SKA3 | 0.017743 |
| FLRT1 | 0.017734 |
| KLK2 | 0.017733 |
| ADD2 | 0.017732 |
| XRCC2 | 0.017706 |
| ELFN2 | 0.017702 |
| SYNPR | 0.017695 |
| KLK12 | 0.017690 |
| CLSPN | 0.017688 |
| SIX6 | 0.017653 |
| KCNT1 | 0.017649 |
| OTP | 0.017637 |
| ANK1 | 0.017597 |
| BRINP2 | 0.017595 |
| KDM5D | 0.017573 |
| MAGEA2 | 0.017564 |
| CEL | 0.017536 |
| KCNC2 | 0.017532 |
| TICRR | 0.017525 |
| ZNF711 | 0.017519 |
| HOXD3 | 0.017504 |
| SPC24 | 0.017477 |
| GRIK5 | 0.017450 |
| PCSK1N | 0.017441 |
| SLC17A6 | 0.017424 |
| RGS7 | 0.017418 |
| ALX3 | 0.017411 |
| CDC20 | 0.017377 |
| NEB | 0.017365 |
| WNK3 | 0.017353 |
| ADCY2 | 0.017343 |
| MKI67 | 0.017323 |
| ORC6 | 0.017320 |
| SPC25 | 0.017314 |
| FAM72C | 0.017257 |
| CELF4 | 0.017256 |
| AURKB | 0.017243 |
| DNASE1L2 | 0.017238 |
| CDCA2 | 0.017232 |
| CYB561D2 | 0.017220 |
| INSM2 | 0.017207 |
| SLC38A11 | 0.017198 |
| RECQL4 | 0.017194 |
| SPTBN4 | 0.017177 |
| KIFC1 | 0.017161 |
| RAX | 0.017156 |
| GAP43 | 0.017144 |
| CDK5R1 | 0.017131 |
| SYT1 | 0.017092 |
| DPY19L2 | 0.017090 |
| T | 0.017089 |
| SCML2 | 0.017083 |
| MCM10 | 0.017074 |
| KIAA1549L | 0.017069 |
| KREMEN2 | 0.017066 |
| OTOG | 0.017059 |
| CCNB2 | 0.017029 |
| PPP2R2C | 0.017019 |
| ZYG11A | 0.017019 |
| CENPE | 0.017014 |
| USP41 | 0.016986 |
| SRRM3 | 0.016932 |
| HCN3 | 0.016911 |
| SP9 | 0.016909 |
| PRC1 | 0.016879 |
| TFAP2B | 0.016877 |
| FSTL5 | 0.016874 |
| CDC45 | 0.016862 |
| CDKN3 | 0.016858 |
| ERCC6L | 0.016858 |
| MGAT5B | 0.016838 |
| HIST1H2BO | 0.016831 |
| PGAM2 | 0.016820 |
| HELLS | 0.016803 |
| GFY | 0.016782 |
| HOXA9 | 0.016780 |
| HIST1H4E | 0.016766 |
| CDCA8 | 0.016753 |
| ADGRD2 | 0.016753 |
| SHISA7 | 0.016741 |
| RTL1 | 0.016737 |
| DRAXIN | 0.016727 |
| BRIP1 | 0.016714 |
| GINS2 | 0.016711 |
| GPR12 | 0.016698 |
| WDR62 | 0.016689 |
| HCN2 | 0.016682 |
| ATP2B2 | 0.016653 |
| FEV | 0.016618 |
| FOXO6 | 0.016602 |
| SYNGR4 | 0.016588 |
| NAT8L | 0.016560 |
| FMN2 | 0.016554 |
| GP2 | 0.016551 |
| CENPI | 0.016549 |
| FAM72B | 0.016547 |
| PRDM13 | 0.016546 |
| MAGED4 | 0.016525 |
| SNCB | 0.016521 |
| SFTPC | −0.049137 |
| SFTPA1 | −0.044867 |
| SFTPA2 | −0.041274 |
| NDUFA7 | −0.038592 |
| SFTA2 | −0.037941 |
| SFTPB | −0.037152 |
| PGC | −0.036730 |
| SLC34A2 | −0.036437 |
| PCDH20 | −0.034771 |
| MSLN | −0.034377 |
| SCGB1A1 | −0.033482 |
| SCEL | −0.033338 |
| SFTPD | −0.032991 |
| SCGB3A2 | −0.032459 |
| NAPSA | −0.032301 |
| SCGB3A1 | −0.032282 |
| GGTLC1 | −0.031540 |
| C4BPA | −0.031076 |
| DMBT1 | −0.030938 |
| PEBP4 | −0.030870 |
| CYP4B1 | −0.030385 |
| MCEMP1 | −0.030373 |
| GPRC5A | −0.030127 |
| PIGR | −0.029974 |
| C16orf89 | −0.029663 |
| AQP4 | −0.029183 |
| MARCO | −0.029014 |
| MUC21 | −0.028905 |
| ROS1 | −0.028783 |
| FOLR1 | −0.028416 |
| LGI3 | −0.028047 |
| TPSB2 | −0.027882 |
| WIF1 | −0.027701 |
| AGTR2 | −0.027492 |
| TPSAB1 | −0.027421 |
| SLC6A14 | −0.027418 |
| AMY1C | −0.027356 |
| CTSE | −0.027329 |
| GKN2 | −0.027021 |
| KRT4 | −0.026975 |
| CEACAM6 | −0.026889 |
| UPK3B | −0.026718 |
| CXCL17 | −0.026697 |
| NDNF | −0.026596 |
| CES1 | −0.026311 |
| SLPI | −0.026184 |
| PLA2G1B | −0.026059 |
| C19orf33 | −0.025997 |
| NOTCH2NL | −0.025885 |
| CPA3 | −0.025846 |
| TNNC1 | −0.025531 |
| SDR16C5 | −0.025341 |
| BPIFB1 | −0.025089 |
| SLC46A2 | −0.025004 |
| CLDN18 | −0.024795 |
| MS4A15 | −0.024495 |
| ALPP | −0.024401 |
| TCF21 | −0.024208 |
| AGER | −0.024176 |
| OLR1 | −0.023965 |
| CST6 | −0.023877 |
| RETN | −0.023868 |
| DPCR1 | −0.023820 |
| CLIC3 | −0.023723 |
| ICAM4 | −0.023711 |
| S100A14 | −0.023674 |
| WNT7A | −0.023672 |
| GDF10 | −0.023584 |
| ANKRD1 | −0.023574 |
| RASGRF1 | −0.023524 |
| CPB2 | −0.023507 |
| FGG | −0.023493 |
| SLC22A31 | −0.023313 |
| SCARA5 | −0.023264 |
| AQP3 | −0.023172 |
| FABP4 | −0.023078 |
| ADH1B | −0.023061 |
| SUSD2 | −0.023013 |
| SLC6A4 | −0.022884 |
| TBX4 | −0.022883 |
| CD300LG | −0.022741 |
| C8B | −0.022649 |
| KCNJ15 | −0.022524 |
| ANXA8 | −0.022470 |
| GPA33 | −0.022447 |
| ALOX15B | −0.022430 |
| ITGB6 | −0.022413 |
| HHIP | −0.022383 |
| FGFBP1 | −0.022319 |
| SEC14L6 | −0.022237 |
| LAMB3 | −0.022222 |
| AQP5 | −0.022201 |
| SOSTDC1 | −0.022189 |
| AREG | −0.022186 |
| FCN3 | −0.022133 |
| HNF1B | −0.022090 |
| TMPRSS2 | −0.022039 |
| MS4A2 | −0.021967 |
| CAV1 | −0.021931 |
| AGR3 | −0.021930 |
| ATP13A4 | −0.021902 |
| PDZK1IP1 | −0.021825 |
| ANXA8L1 | −0.021694 |
| CHI3L2 | −0.021649 |
| ADAMTS8 | −0.021583 |
| MMP28 | −0.021581 |
| HLA-DRB5 | −0.021553 |
| CNTN6 | −0.021551 |
| VEPH1 | −0.021507 |
| FCER1A | −0.021492 |
| ITLN2 | −0.021478 |
| LRRN4 | −0.021477 |
| CFTR | −0.021445 |
| SLC19A3 | −0.021412 |
| PTGDS | −0.021317 |
| DES | −0.021311 |
| INMT | −0.021287 |
| DAW1 | −0.021279 |
| HPGD | −0.021259 |
| SCTR | −0.021220 |
| IL1RL1 | −0.021193 |
| LRRK2 | −0.021177 |
| CLIC5 | −0.021171 |
| GALNT5 | −0.021097 |
| SERPINA1 | −0.021091 |
| MUC15 | −0.021057 |
| CCL23 | −0.020920 |
| F11 | −0.020907 |
| STEAP4 | −0.020716 |
| ST6GALNAC1 | −0.020708 |
| APOH | −0.020622 |
| SDPR | −0.020600 |
| ALOX5AP | −0.020596 |
| ANXA3 | −0.020557 |
| C3 | −0.020551 |
| COL6A5 | −0.020506 |
| PLA2G4F | −0.020487 |
| CXCL5 | −0.020457 |
| HOPX | −0.020316 |
| TMEM100 | −0.020247 |
| ZBED2 | −0.020165 |
| FOSB | −0.020112 |
| CCL17 | −0.020065 |
| MRC1 | −0.020063 |
| MALL | −0.020045 |
| CSF3 | −0.020038 |
| WNT3A | −0.020016 |
| CAV2 | −0.020013 |
| SH3RF2 | −0.019994 |
| VGLL1 | −0.019975 |
| LAMP3 | −0.019949 |
| FOLR3 | −0.019904 |
| XAGE2 | −0.019902 |
| TREM1 | −0.019900 |
| DPP4 | −0.019878 |
| GSTA1 | −0.019745 |
| CLEC3B | −0.019670 |
| GPM6A | −0.019613 |
| FAM107A | −0.019607 |
| LCN2 | −0.019592 |
| WISP2 | −0.019587 |
| CCL22 | −0.019553 |
| GPIHBP1 | −0.019542 |
| FAM216B | −0.019483 |
| SLC22A3 | −0.019466 |
| LIPH | −0.019455 |
| CCL21 | −0.019437 |
| TMEM92 | −0.019416 |
| ARSE | −0.019412 |
| CHIA | −0.019366 |
| MFAP4 | −0.019360 |
| FPR2 | −0.019339 |
| TACSTD2 | −0.019333 |
| ALDH3B1 | −0.019325 |
| MYOC | −0.019323 |
| PCSK9 | −0.019290 |
| ICAM1 | −0.019256 |
| RRAD | −0.019241 |
| FXYD1 | −0.019213 |
| MUC1 | −0.019101 |
| C20orf85 | −0.019046 |
| FMO2 | −0.019028 |
| RSPO2 | −0.019020 |
| KRT7 | −0.019005 |
| SCN7A | −0.018972 |
| CD52 | −0.018928 |
| IL7R | −0.018912 |
| AQP1 | −0.018856 |
| HLA-DQB2 | −0.018804 |
| VSIG4 | −0.018795 |
| VSIG2 | −0.018792 |
| CD1C | −0.018780 |
| ALPPL2 | −0.018779 |
| RNASE1 | −0.018771 |
| HCAR2 | −0.018748 |
| PTGS2 | −0.018732 |
| C1orf116 | −0.018713 |
| SLC39A8 | −0.018665 |
| FCN1 | −0.018617 |
| RSPO1 | −0.018610 |
| RS1 | −0.018588 |
| FLRT3 | −0.018574 |
| SGCA | −0.018533 |
| DUOXA1 | −0.018529 |
| CTSH | −0.018528 |
| CXCL2 | −0.018506 |
| HHLA2 | −0.018498 |
| AADAC | −0.018447 |
| CAPN8 | −0.018437 |
| CHRDL1 | −0.018388 |
| P3H2 | −0.018382 |
| CYP2S1 | −0.018375 |
| CCL14 | −0.018313 |
| BMP3 | −0.018298 |
| KLK10 | −0.018290 |
| MAP1LC3C | −0.018255 |
| ITGB4 | −0.018226 |
| TUBB3 | −0.018181 |
| CYS1 | −0.018145 |
| KLK7 | −0.018119 |
| C1orf167 | −0.018098 |
| SIRPB1 | −0.018058 |
| SHH | −0.018037 |
| SCN2B | −0.018037 |
| C7 | −0.017977 |
| OSCAR | −0.017960 |
| CXCR2 | −0.017956 |
| APOC2 | −0.017947 |
| IYD | −0.017945 |
| ADGRD1 | −0.017944 |
| ALPL | −0.017943 |
| HPGDS | −0.017937 |
| ADGRE3 | −0.017937 |
| S100A4 | −0.017855 |
| ADGRE1 | −0.017848 |
| PPBP | −0.017789 |
| SEC14L4 | −0.017780 |
| CPAMD8 | −0.017709 |
| FGA | −0.017698 |
| MUC5B | −0.017685 |
| EDN1 | −0.017638 |
| NTF4 | −0.017601 |
| DPT | −0.017561 |
| AQP9 | −0.017540 |
| FBLN5 | −0.017535 |
| AGR2 | −0.017498 |
| PDPN | −0.017496 |
| HYAL1 | −0.017485 |
| WDR38 | −0.017483 |
| SNTN | −0.017477 |
| KNDC1 | −0.017464 |
| S100A10 | −0.017458 |
| HRASLS5 | −0.017443 |
| NINJ2 | −0.017424 |
| CD55 | −0.017413 |
| DRAM1 | −0.017406 |
| GSTM5 | −0.017390 |
| LAMC2 | −0.017377 |
| CD74 | −0.017314 |
| ALDH3A1 | −0.017290 |
| HKDC1 | −0.017251 |
| GPX3 | −0.017251 |
| TNXB | −0.017236 |
| GPD1 | −0.017233 |
| AOC3 | −0.017232 |
| OGN | −0.017222 |
| ANKRD29 | −0.017213 |
| BMP5 | −0.017207 |
| PRG4 | −0.017161 |
| EHD2 | −0.017157 |
| CAPSL | −0.017128 |
| AKR1C3 | −0.017116 |
| PRSS8 | −0.017085 |
| PI16 | −0.017077 |
| TMEM213 | −0.017072 |
| LRG1 | −0.017071 |
| C11orf88 | −0.017069 |
| HLA-DRB1 | −0.017058 |
| ITLN1 | −0.017050 |
| C6 | −0.017049 |
| EVPLL | −0.017036 |
| CTSG | −0.017006 |
| EFEMP1 | −0.017000 |
| DUOX1 | −0.016986 |
| CD1E | −0.016972 |
| ADH1C | −0.016969 |
| SIGLEC6 | −0.016962 |
| CPM | −0.016927 |
| PRX | −0.016925 |
| CD68 | −0.016872 |
| B3GNT7 | −0.016822 |
| SLC16A5 | −0.016785 |
| SPN | −0.016782 |
| WNT2 | −0.016770 |
| DKK2 | −0.016758 |
| S100A9 | −0.016754 |
| LYVE1 | −0.016734 |
| ADGRF1 | −0.016700 |
| LYZ | −0.016687 |
| FBP1 | −0.016669 |
| ATOH8 | −0.016642 |
| MAMDC2 | −0.016640 |
| STX11 | −0.016626 |
| SELP | −0.016609 |
| HLA-DRA | −0.016601 |
| CH25H | −0.016552 |
| SGMS2 | −0.016549 |
| TEKT1 | −0.016543 |
| MYZAP | −0.016540 |
| ELF5 | −0.016456 |
| WFDC12 | −0.016428 |
| HP | −0.016414 |
| HSPB6 | −0.016411 |
| ADGRE5 | −0.016360 |
| CXCR1 | −0.016350 |
| C2orf54 | −0.016346 |
| HLA-DQA1 | −0.016339 |
| ADTRP | −0.016334 |
| CCDC170 | −0.016332 |
| ADIRF | −0.016310 |
| SCNN1B | −0.016302 |
| CD300C | −0.016272 |
| COLEC12 | −0.016264 |
| HCAR3 | −0.016261 |
| CCL13 | −0.016253 |
| FOXQ1 | −0.016233 |
| ACKR1 | −0.016226 |
| CCDC68 | −0.016220 |
| HLA-DOA | −0.016206 |
| PLA2G10 | −0.016203 |
| STOML3 | −0.016201 |
| FGFR4 | −0.016127 |
| C1QTNF7 | −0.016095 |
| ADRB1 | −0.016091 |
| FPR1 | −0.016087 |
| RNF128 | −0.016064 |
| ANGPT1 | −0.016041 |
| NFE2 | −0.016025 |
| HLA-DQB1 | −0.016015 |
| CCL18 | −0.016010 |
| GGT6 | −0.015994 |
| JCHAIN | −0.015985 |
| MSR1 | −0.015978 |
| HBB | −0.015965 |
| GJA5 | −0.015951 |
| CXCL16 | −0.015930 |
| ADCY8 | −0.015925 |
| CST5 | −0.015922 |
| FAM81B | −0.015898 |
| FGF10 | −0.015888 |
| IL33 | −0.015862 |
| RBP4 | −0.015849 |
| ADRB2 | −0.015848 |
| RAB27B | −0.015843 |
| IL1A | −0.015822 |
| SLC51B | −0.015789 |
| CFD | −0.015788 |
| HLA-DPA1 | −0.015784 |
| IRX6 | −0.015782 |
| LGALS3 | −0.015781 |
| FAM189A2 | −0.015781 |
| ALDH2 | −0.015779 |
| HLA-DQA2 | −0.015779 |
| PRODH | −0.015775 |
| S100A8 | −0.015720 |
| IL6 | −0.015712 |
| CNN2 | −0.015692 |
| PGM5 | −0.015672 |
| ERP27 | −0.015669 |
| EREG | −0.015652 |
| TREM2 | −0.015641 |
| S1PR4 | −0.015630 |
| SYT8 | −0.015594 |
| HLA-DPB1 | −0.015588 |
| ABI3BP | −0.015577 |
| EPAS1 | −0.015576 |
| CLDN2 | −0.015544 |
| DLC1 | −0.015534 |
| FIBIN | −0.015533 |
| PLBD1 | −0.015532 |
| CD1A | −0.015494 |
| MYRF | −0.015476 |
| CSTA | −0.015470 |
| ORM1 | −0.015450 |
| CCDC69 | −0.015415 |
| C2 | −0.015413 |
| SYNC | −0.015399 |
| CLEC10A | −0.015384 |
| NEDD9 | −0.015375 |
| DPEP2 | −0.015368 |
| HBA2 | −0.015366 |
| ABCA8 | −0.015365 |
| C2orf71 | −0.015365 |
| PRSS21 | −0.015360 |
| SELPLG | −0.015331 |
| PAPSS2 | −0.015329 |
| MYH2 | −0.015319 |
| GYPE | −0.015291 |
| CA4 | −0.015265 |
| KRT13 | −0.015264 |
| DCN | −0.015230 |
| DCSTAMP | −0.015220 |
| CSF3R | −0.015218 |
| THBD | −0.015178 |
| DKK1 | −0.015170 |
| ALOX15 | −0.015139 |
| CX3CR1 | −0.015138 |
| TNNI2 | −0.015138 |
| MNDA | −0.015138 |
| CRTAC1 | −0.015136 |
| TGFBR2 | −0.015107 |
| LDLRAD1 | −0.015104 |
| FCGR3B | −0.015092 |
| CX3CL1 | −0.015087 |
| HRCT1 | −0.015083 |
| EPHA2 | −0.015061 |
| PPARG | −0.015036 |
| FGFR2 | −0.015034 |
| CTSW | −0.015021 |
| TMEM139 | −0.015019 |
| CYR61 | −0.014969 |
| TFPI | −0.014968 |
| SHISA3 | −0.014966 |
| NPR1 | −0.014960 |
| GPR39 | −0.014948 |
| CHIT1 | −0.014947 |
| ACSL5 | −0.014931 |
| FOXF1 | −0.014919 |
| SIGLEC11 | −0.014916 |
| PLA2G4A | −0.014887 |
| A2M | −0.014877 |
| ST8SIA6 | −0.014871 |
| ODAM | −0.014870 |
| HLA-DMA | −0.014862 |
| ITGA8 | −0.014846 |
| GSTA2 | −0.014834 |
| SULT1A4 | −0.014828 |
| SHE | −0.014828 |
| MATN3 | −0.014816 |
| COL4A3 | −0.014803 |
| CFAP221 | −0.014802 |
| B3GNT3 | −0.014796 |
| CLEC4E | −0.014792 |
| TFPI2 | −0.014790 |
| SFTA3 | −0.014790 |
| SLC11A1 | −0.014768 |
| RGCC | −0.014766 |
| ADGRF5 | −0.014755 |
| ABCC6 | −0.014750 |
| SERPINA3 | −0.014729 |
| SPOCK2 | −0.014729 |
| TLCD2 | −0.014721 |
| CSF2 | −0.014721 |
| TEK | −0.014710 |
| TMEM190 | −0.014708 |
| ITGAM | −0.014690 |
| F3 | −0.014689 |
| CATSPER1 | −0.014675 |
| LILRA2 | −0.014672 |
| TMEM212 | −0.014669 |
| MAOA | −0.014655 |
| CD69 | −0.014636 |
| MT1A | −0.014634 |
| NCF2 | −0.014617 |
| HSPB8 | −0.014584 |
| SGCG | −0.014570 |
| SHROOM4 | −0.014561 |
| ANXA1 | −0.014553 |
| IL1RN | −0.014550 |
| LY86 | −0.014549 |
| CYBRD1 | −0.014549 |
| P2RY2 | −0.014545 |
| SELENBP1 | −0.014538 |
| SLCO2A1 | −0.014514 |
| HBEGF | −0.014442 |
| SYT15 | −0.014433 |
| ACP5 | −0.014416 |
| CCBE1 | −0.014404 |
| MYH11 | −0.014346 |
| DNAH9 | −0.014346 |
| AKR1C2 | −0.014336 |
| TYROBP | −0.014333 |
| SULT2B1 | −0.014324 |
| MPZL2 | −0.014319 |
| PTAFR | −0.014316 |
| ZFP36 | −0.014307 |
| SULT1A2 | −0.014291 |
| LILRA5 | −0.014282 |
| IL6R | −0.014276 |
| SYNDIG1L | −0.014249 |
| NTN4 | −0.014244 |
| CLEC12A | −0.014230 |
| TIMP3 | −0.014230 |
| TLR8 | −0.014217 |
| RAMP3 | −0.014199 |
| TTC29 | −0.014195 |
| MALRD1 | −0.014188 |
| TNIP3 | −0.014183 |
| DOK2 | −0.014170 |
| CREB3L1 | −0.014159 |

Tables 4B and 5B are based on PRISM data analysis. PRISM data was downloaded from https://depmap.org/(20Q1). For PLSR analyses, lung small cell and lung adenocarcinoma samples were extracted, and missing values were filled by kNN imputation, using k=10, done on these lung samples only. PLSR was performed using the R package mixOmics, and varimax rotation was applied to the first two components. Loadings (consisting of drug names) were ranked by the first component, and enrichment of drug targets based on this ranking was assessed using signed KS test. Only drug targets with frequency>8 were included in the list.

TABLE 4A
GDSC-based Drugs with sensitivity in SCN, SRBCT or Blood cancer types

| | | | | | | | | | | (SCN & | Sensitivity |
|---|---|---|---|---|---|---|---|---|---|---|---|
| | SCN - | | | | | | SCN | SRBCT | SCN | SRBCT) | in |
| | SCLC vs | | | | | | & | & | & | & | SCN, |
| | LUAD lung | | SRBCT vs | | Blood vs | | Blood | Blood | SRBCT | BLOOD | SRBCT |
| | cancer | | OTHER | | OTHER | | avg. | avg. | avg. | avg. | or |
| DRUG NAME | signlogp | rank | signlogp | rank | signlogp | rank | rank | rank | rank | rank | Blood |
| ABT-263 | 10.25 | 1 | 5.70 | 46 | 22.72 | 77 | 39 | 61.5 | 23.5 | 50.25 | 1 |
| NSC-207895 | 9.76 | 2 | 1.41 | 126 | 19.39 | 88 | 45 | 107 | 64 | 76 | 1 |
| NPK76-II-72-1 | 8.76 | 3 | 8.98 | 10 | 48.24 | 9 | 6 | 9.5 | 6.5 | 7.75 | 1 |
| XMD13-2 | 8.72 | 4 | 7.83 | 21 | 41.47 | 24 | 14 | 22.5 | 12.5 | 18.25 | 1 |
| MP470 | 8.10 | 5 | 3.71 | 73 | 13.96 | 108 | 56.5 | 90.5 | 39 | 73.5 | 1 |
| BX-912 | 7.20 | 6 | 7.61 | 23 | 31.30 | 49 | 27.5 | 36 | 14.5 | 31.75 | 1 |
| GW-2580 | 7.04 | 7 | 1.51 | 124 | 15.91 | 99 | 53 | 111.5 | 65.5 | 82.25 | 1 |
| GSK1070916 | 6.43 | 8 | 8.57 | 14 | 22.09 | 79 | 43.5 | 46.5 | 11 | 45 | 1 |
| WZ3105 | 6.32 | 9 | 2.01 | 109 | 39.58 | 28 | 18.5 | 68.5 | 59 | 43.5 | 1 |
| GSK690693 | 6.14 | 10 | 2.32 | 98 | 19.88 | 86 | 48 | 92 | 54 | 70 | 1 |
| OSI-027 | 5.97 | 11 | 3.84 | 70 | 44.33 | 20 | 15.5 | 45 | 40.5 | 30.25 | 1 |
| FK866 | 5.66 | 12 | 6.06 | 40 | 29.93 | 54 | 33 | 47 | 26 | 40 | 1 |
| I-BET-762 | 5.36 | 13 | 8.85 | 11 | 46.96 | 13 | 13 | 12 | 12 | 12.5 | 1 |
| GSK429286A | 5.34 | 14 | 5.66 | 47 | 32.00 | 46 | 30 | 46.5 | 30.5 | 38.25 | 1 |
| UNC0638 | 5.29 | 15 | 6.42 | 36 | 76.46 | 1 | 8 | 18.5 | 25.5 | 13.25 | 1 |
| PHA-793887 | 5.25 | 16 | 3.11 | 84 | 49.27 | 5 | 10.5 | 44.5 | 50 | 27.5 | 1 |
| QL-X-138 | 5.20 | 17 | 4.96 | 53 | 39.05 | 30 | 23.5 | 41.5 | 35 | 32.5 | 1 |
| Vorinostat | 5.19 | 18 | 11.84 | 4 | 48.37 | 7 | 12.5 | 5.5 | 11 | 9 | 1 |
| Tubastatin A | 5.06 | 19 | 8.41 | 16 | 47.71 | 10 | 14.5 | 13 | 17.5 | 13.75 | 1 |
| CX-5461 | 5.05 | 20 | 10.30 | 6 | 26.92 | 67 | 43.5 | 36.5 | 13 | 40 | 1 |
| THZ-2-102-1 | 4.92 | 21 | 4.18 | 63 | 43.09 | 21 | 21 | 42 | 42 | 31.5 | 1 |
| KIN001-270 | 4.47 | 22 | 7.60 | 24 | 35.57 | 36 | 29 | 30 | 23 | 29.5 | 1 |
| SB52334 | 4.29 | 23 | 14.22 | 2 | 11.88 | 119 | 71 | 60.5 | 12.5 | 65.75 | 1 |
| PI-103 | 4.20 | 24 | 3.42 | 78 | 36.81 | 35 | 29.5 | 56.5 | 51 | 43 | 1 |
| QL-XI-92 | 4.18 | 25 | 4.72 | 55 | 39.94 | 26 | 25.5 | 40.5 | 40 | 33 | 1 |
| AC220 | 3.92 | 26 | 6.46 | 33 | 16.57 | 97 | 61.5 | 65 | 29.5 | 63.25 | 1 |
| PIK-93 | 3.91 | 27 | 4.40 | 58 | 38.99 | 31 | 29 | 44.5 | 42.5 | 36.75 | 1 |
| TL-2-105 | 3.85 | 28 | 5.65 | 48 | 31.82 | 47 | 37.5 | 47.5 | 38 | 42.5 | 1 |
| Belinostat | 3.74 | 29 | 5.99 | 41 | 27.08 | 65 | 47 | 53 | 35 | 50 | 1 |
| BMS-345541 | 3.71 | 30 | 4.37 | 60 | 48.43 | 6 | 18 | 33 | 45 | 25.5 | 1 |
| XMD14-99 | 3.68 | 31 | 9.02 | 9 | 38.35 | 32 | 31.5 | 20.5 | 20 | 26 | 1 |
| Masitinib | 3.66 | 32 | 9.05 | 8 | 29.57 | 55 | 43.5 | 31.5 | 20 | 37.5 | 1 |
| JW-7-24-1 | 3.64 | 33 | 6.13 | 37 | 48.36 | 8 | 20.5 | 22.5 | 35 | 21.5 | 1 |
| KIN001-260 | 3.56 | 34 | 7.52 | 25 | 51.85 | 3 | 18.5 | 14 | 29.5 | 16.25 | 1 |
| AR-42 | 3.38 | 35 | 7.42 | 27 | 33.61 | 41 | 38 | 34 | 31 | 36 | 1 |
| AV-951 | 3.38 | 36 | 4.47 | 57 | 21.33 | 82 | 59 | 69.5 | 46.5 | 64.25 | 1 |
| CAY10603 | 3.31 | 37 | 14.11 | 3 | 32.43 | 43 | 40 | 23 | 20 | 31.5 | 1 |
| SB-505124 | 3.28 | 38 | 3.33 | 80 | 18.10 | 93 | 65.5 | 86.5 | 59 | 76 | 1 |
| TG101348 | 3.26 | 39 | 8.19 | 18 | 45.95 | 16 | 27.5 | 17 | 28.5 | 22.25 | 1 |
| SB-715992 | 3.23 | 40 | 5.88 | 43 | 42.83 | 22 | 31 | 32.5 | 41.5 | 31.75 | 1 |
| Methotrexate | 3.23 | 41 | 3.71 | 72 | 47.62 | 12 | 26.5 | 42 | 56.5 | 34.25 | 1 |
| QL-XII-47 | 3.07 | 42 | 1.66 | 119 | 32.33 | 44 | 43 | 81.5 | 80.5 | 62.25 | 1 |
| KIN001-244 | 3.05 | 43 | 5.74 | 45 | 28.90 | 58 | 50.5 | 51.5 | 44 | 51 | 1 |
| NG-25 | 2.89 | 44 | 6.72 | 30 | 27.50 | 62 | 53 | 46 | 37 | 49.5 | 1 |
| Ruxolitinib | 2.88 | 45 | 3.90 | 68 | 18.74 | 91 | 68 | 79.5 | 56.5 | 73.75 | 1 |
| KIN001-236 | 2.80 | 46 | 6.45 | 35 | 45.62 | 18 | 32 | 26.5 | 40.5 | 29.25 | 1 |
| UNC1215 | 2.71 | 47 | 3.81 | 71 | 45.74 | 17 | 32 | 44 | 59 | 38 | 1 |
| Y-39983 | 2.58 | 48 | 5.95 | 42 | 32.33 | 45 | 46.5 | 43.5 | 45 | 45 | 1 |
| T0901317 | 2.52 | 49 | 3.52 | 77 | 32.70 | 42 | 45.5 | 59.5 | 63 | 52.5 | 1 |
| Zibotentan | 2.33 | 50 | 4.07 | 64 | 34.08 | 39 | 44.5 | 51.5 | 57 | 48 | 1 |
| ABT-869 | 2.27 | 51 | 2.03 | 106 | 9.62 | 128 | 89.5 | 117 | 78.5 | 103.25 | 1 |
| 5-Fluorouracil | 2.24 | 52 | 8.78 | 13 | 31.14 | 50 | 51 | 31.5 | 32.5 | 41.25 | 1 |
| Temozolomide | 2.22 | 53 | 1.30 | 130 | 26.93 | 66 | 59.5 | 98 | 91.5 | 78.75 | 1 |
| Genentech Cpd 10 | 2.20 | 54 | 7.99 | 19 | 34.16 | 38 | 46 | 28.5 | 36.5 | 37.25 | 1 |
| GSK2126458 | 2.16 | 55 | 1.80 | 116 | 22.29 | 78 | 66.5 | 97 | 85.5 | 81.75 | 1 |
| CUDC-101 | 2.13 | 56 | 4.38 | 59 | 28.04 | 59 | 57.5 | 59 | 57.5 | 58.25 | 1 |
| VX-702 | 2.04 | 57 | 2.90 | 87 | 31.65 | 48 | 52.5 | 67.5 | 72 | 60 | 1 |
| SNX-2112 | 2.03 | 58 | 4.19 | 62 | 50.88 | 4 | 31 | 33 | 60 | 32 | 1 |
| XMD15-27 | 2.00 | 59 | 3.63 | 75 | 30.75 | 52 | 55.5 | 63.5 | 67 | 59.5 | 1 |
| XL-880 | 1.63 | 60 | 5.80 | 44 | 14.43 | 106 | 83 | 75 | 52 | 79 | 1 |
| Axitinib | 1.63 | 61 | 5.32 | 51 | 9.49 | 130 | 95.5 | 90.5 | 56 | 93 | 1 |
| BIX02189 | 1.61 | 62 | 6.11 | 38 | 42.52 | 23 | 42.5 | 30.5 | 50 | 36.5 | 1 |
| TL-1-85 | 1.60 | 63 | 7.22 | 28 | 29.29 | 56 | 59.5 | 42 | 45.5 | 50.75 | 1 |
| PD-173074 | 1.56 | 64 | 0.33 | 163 | 15.08 | 104 | 84 | 133.5 | 113.5 | 108.75 | 1 |
| AT-7519 | 1.55 | 65 | 1.99 | 110 | 46.47 | 14 | 39.5 | 62 | 87.5 | 50.75 | 1 |
| TPCA-1 | 1.54 | 66 | 11.56 | 5 | 44.68 | 19 | 42.5 | 12 | 35.5 | 27.25 | 1 |
| STF-62247 | 1.52 | 67 | 1.98 | 111 | 37.48 | 33 | 50 | 72 | 89 | 61 | 1 |
| VNLG/124 | 1.46 | 68 | 6.46 | 34 | 24.64 | 73 | 70.5 | 53.5 | 51 | 62 | 1 |
| QL-XII-61 | 1.36 | 69 | 1.63 | 120 | 10.39 | 124 | 96.5 | 122 | 94.5 | 109.25 | 1 |
| YM201636 | 1.35 | 70 | 7.71 | 22 | 39.68 | 27 | 48.5 | 24.5 | 46 | 36.5 | 1 |
| LAQ824 | 1.35 | 71 | 1.05 | 139 | 16.52 | 98 | 84.5 | 118.5 | 105 | 101.5 | 1 |
| Nilotinib | 1.23 | 72 | 1.26 | 132 | 12.94 | 114 | 93 | 123 | 102 | 108 | 1 |
| Vismodegib | 1.14 | 73 | 2.34 | 97 | 26.15 | 70 | 71.5 | 83.5 | 85 | 77.5 | 1 |
| EX-527 | 1.10 | 74 | 3.90 | 66 | 34.34 | 37 | 55.5 | 51.5 | 70 | 53.5 | 1 |
| MPS-1-IN-1 | 1.09 | 75 | 14.35 | 1 | 37.38 | 34 | 54.5 | 17.5 | 38 | 36 | 1 |
| Phenformin | 0.96 | 78 | −1.32 | 220 | 27.48 | 63 | 70.5 | 141.5 | 149 | 106 | 1 |
| FMK | 0.88 | 80 | 2.64 | 92 | 27.74 | 60 | 70 | 76 | 86 | 73 | 1 |
| Olaparib | 0.85 | 82 | 9.23 | 7 | 17.34 | 94 | 88 | 50.5 | 44.5 | 69.25 | 1 |
| CP466722 | 0.84 | 83 | 8.42 | 15 | 47.64 | 11 | 47 | 13 | 49 | 30 | 1 |
| KIN001-135 | 0.78 | 84 | 1.23 | 133 | 13.12 | 112 | 98 | 122.5 | 108.5 | 110.25 | 1 |
| Lenalidomide | 0.76 | 85 | 2.92 | 86 | 26.54 | 69 | 77 | 77.5 | 85.5 | 77.25 | 1 |
| Cyclopamine | 0.63 | 86 | −2.68 | 237 | 11.89 | 118 | 102 | 177.5 | 161.5 | 139.75 | 1 |
| SGC0946 | 0.63 | 87 | 3.32 | 81 | 30.39 | 53 | 70 | 67 | 84 | 68.5 | 1 |
| ZM-447439 | 0.63 | 88 | 3.41 | 79 | 9.68 | 127 | 107.5 | 103 | 83.5 | 105.25 | 1 |
| SL 0101-1 | 0.58 | 91 | 1.78 | 117 | 18.30 | 92 | 91.5 | 104.5 | 104 | 98 | 1 |
| KIN001-266 | 0.55 | 93 | 7.88 | 20 | 39.07 | 29 | 61 | 24.5 | 56.5 | 42.75 | 1 |
| NVP-BHG712 | 0.53 | 94 | 2.36 | 96 | 27.52 | 61 | 77.5 | 78.5 | 95 | 78 | 1 |
| XL-184 | 0.52 | 95 | 2.95 | 85 | 13.19 | 111 | 103 | 98 | 90 | 100.5 | 1 |
| CAL-101 | 0.49 | 96 | 2.03 | 107 | 23.26 | 75 | 85.5 | 91 | 101.5 | 88.25 | 1 |
| Camptothecin | 0.47 | 97 | 7.08 | 29 | 10.23 | 126 | 111.5 | 77.5 | 63 | 94.5 | 1 |
| KIN001-102 | 0.47 | 98 | 4.06 | 65 | 40.59 | 25 | 61.5 | 45 | 81.5 | 53.25 | 1 |
| ABT-888 | 0.47 | 99 | 2.90 | 88 | 23.24 | 76 | 87.5 | 82 | 93.5 | 84.75 | 1 |
| AICAR | 0.45 | 100 | 0.03 | 178 | 33.97 | 40 | 70 | 109 | 139 | 89.5 | 1 |
| Cisplatin | 0.41 | 102 | 4.51 | 56 | 2.01 | 199 | 150.5 | 127.5 | 79 | 139 | 1 |
| EHT 1864 | 0.40 | 103 | 1.91 | 113 | 12.58 | 117 | 110 | 115 | 108 | 112.5 | 1 |
| Bicalutamide | 0.37 | 104 | 1.34 | 128 | 11.83 | 120 | 112 | 124 | 116 | 118 | 1 |
| JQ1 | 0.29 | 110 | 0.30 | 164 | 19.97 | 85 | 97.5 | 124.5 | 137 | 111 | 1 |
| FR-180204 | 0.23 | 113 | 7.43 | 26 | 20.99 | 83 | 98 | 54.5 | 69.5 | 76.25 | 1 |
| Crizotinib | 0.21 | 114 | 0.44 | 159 | 12.83 | 115 | 114.5 | 137 | 136.5 | 125.75 | 1 |
| CEP-701 | 0.18 | 116 | 2.53 | 94 | 17.27 | 96 | 106 | 95 | 105 | 100.5 | 1 |
| OSI-930 | 0.17 | 117 | 4.97 | 52 | 31.12 | 51 | 84 | 51.5 | 84.5 | 67.75 | 1 |
| ZG-10 | 0.15 | 119 | 1.37 | 127 | 7.44 | 143 | 131 | 135 | 123 | 133 | 1 |
| AG-014699 | 0.13 | 120 | 6.56 | 31 | 3.41 | 187 | 153.5 | 109 | 75.5 | 131.25 | 1 |
| TAK-715 | 0.12 | 121 | 4.79 | 54 | 54.97 | 2 | 61.5 | 28 | 87.5 | 44.75 | 1 |
| PF-4708671 | 0.11 | 122 | 0.48 | 157 | 13.22 | 110 | 116 | 133.5 | 139.5 | 124.75 | 1 |
| ATRA | 0.08 | 124 | 3.29 | 82 | 17.33 | 95 | 109.5 | 88.5 | 103 | 99 | 1 |
| CP724714 | 0.08 | 125 | 1.02 | 142 | 10.48 | 122 | 123.5 | 132 | 133.5 | 127.75 | 1 |
| PAC-1 | 0.04 | 127 | 2.83 | 89 | 27.26 | 64 | 95.5 | 76.5 | 108 | 86 | 1 |
| Tamoxifen | 0.04 | 128 | 0.60 | 152 | 26.88 | 68 | 98 | 110 | 140 | 104 | 1 |
| SN-38 | 0.01 | 129 | 3.54 | 76 | 6.00 | 154 | 141.5 | 115 | 102.5 | 128.25 | 1 |
| CH5424802 | 0.00 | 130 | 5.33 | 50 | 15.23 | 103 | 116.5 | 76.5 | 90 | 96.5 | 1 |
| Etoposide | 0.00 | 131 | 2.05 | 105 | 6.59 | 145 | 138 | 125 | 118 | 131.5 | 1 |
| QS11 | −0.01 | 132 | 2.72 | 91 | 13.05 | 113 | 122.5 | 102 | 111.5 | 112.25 | 1 |
| Shikonin | −0.02 | 134 | 1.91 | 112 | 7.23 | 144 | 139 | 128 | 123 | 133.5 | 1 |
| BX-795 | −0.03 | 135 | 2.16 | 101 | 9.16 | 134 | 134.5 | 117.5 | 118 | 126 | 1 |
| JQ12 | −0.06 | 136 | 1.60 | 121 | 8.61 | 137 | 136.5 | 129 | 128.5 | 132.75 | 1 |
| BAY 61-3606 | −0.15 | 143 | 6.53 | 32 | 21.60 | 80 | 111.5 | 56 | 87.5 | 83.75 | 1 |
| ZSTK474 | −0.16 | 145 | 2.46 | 95 | 25.35 | 71 | 108 | 83 | 120 | 95.5 | 1 |
| BMS-708163 | −0.19 | 147 | 2.20 | 100 | 19.03 | 90 | 118.5 | 95 | 123.5 | 106.75 | 1 |
| IPA-3 | −0.30 | 151 | 3.86 | 69 | 25.06 | 72 | 111.5 | 70.5 | 110 | 91 | 1 |
| XMD11-85h | −0.31 | 152 | 2.12 | 103 | 10.43 | 123 | 137.5 | 113 | 127.5 | 125.25 | 1 |
| TW 37 | −0.41 | 154 | 2.02 | 108 | 0.53 | 224 | 189 | 166 | 131 | 177.5 | 1 |
| SB 216763 | −0.46 | 157 | 2.10 | 104 | 7.63 | 142 | 149.5 | 123 | 130.5 | 136.25 | 1 |
| 681640 | −0.47 | 158 | 1.82 | 115 | 4.82 | 167 | 162.5 | 141 | 136.5 | 151.75 | 1 |
| PFI-1 | −0.54 | 162 | 0.15 | 169 | 15.50 | 100 | 131 | 134.5 | 165.5 | 132.75 | 1 |
| LFM-A13 | −0.67 | 166 | 1.59 | 122 | 9.28 | 133 | 149.5 | 127.5 | 144 | 138.5 | 1 |
| DMOG | −0.83 | 172 | 0.05 | 176 | 15.44 | 101 | 136.5 | 138.5 | 174 | 137.5 | 1 |
| BMS-754807 | −0.89 | 174 | 8.79 | 12 | −1.05 | 240 | 207 | 126 | 93 | 166.5 | 1 |
| VX-11e | −0.92 | 175 | −0.07 | 188 | 13.89 | 109 | 142 | 148.5 | 181.5 | 145.25 | 1 |
| AZD8055 | −0.97 | 176 | 0.07 | 175 | 24.64 | 74 | 125 | 124.5 | 175.5 | 124.75 | 1 |
| AMG-706 | −1.04 | 179 | 1.89 | 114 | 0.66 | 221 | 200 | 167.5 | 146.5 | 183.75 | 1 |
| Mitomycin C | −1.06 | 181 | 2.31 | 99 | 3.84 | 179 | 180 | 139 | 140 | 159.5 | 1 |
| OSI-906 | −1.08 | 182 | 8.31 | 17 | 1.04 | 211 | 196.5 | 114 | 99.5 | 155.25 | 1 |
| CCT007093 | −1.10 | 184 | 1.71 | 118 | 14.94 | 105 | 144.5 | 111.5 | 151 | 128 | 1 |
| JNK Inhibitor VIII | −1.10 | 185 | 2.13 | 102 | 5.56 | 160 | 172.5 | 131 | 143.5 | 151.75 | 1 |
| OSU-03012 | −1.17 | 188 | 1.30 | 129 | 12.62 | 116 | 152 | 122.5 | 158.5 | 137.25 | 1 |
| AZD7762 | −1.20 | 190 | 2.60 | 93 | 19.15 | 89 | 139.5 | 91 | 141.5 | 115.25 | 1 |
| BMN-673 | −1.31 | 194 | 6.07 | 39 | 0.95 | 215 | 204.5 | 127 | 116.5 | 165.75 | 1 |
| Gemcitabine | −1.32 | 195 | 1.58 | 123 | 6.20 | 149 | 172 | 136 | 159 | 154 | 1 |
| NU-7441 | −1.33 | 196 | 0.59 | 154 | 10.33 | 125 | 160.5 | 139.5 | 175 | 150 | 1 |
| KU-55933 | −1.34 | 197 | 0.50 | 156 | 20.34 | 84 | 140.5 | 120 | 176.5 | 130.25 | 1 |
| AP-24534 | −1.39 | 200 | −0.17 | 197 | 11.81 | 121 | 160.5 | 159 | 198.5 | 159.75 | 1 |
| AS605240 | −1.39 | 201 | 0.68 | 148 | 19.56 | 87 | 144 | 117.5 | 174.5 | 130.75 | 1 |
| HG-5-113-01 | −1.47 | 204 | 4.20 | 61 | 3.79 | 181 | 192.5 | 121 | 132.5 | 156.75 | 1 |
| Thapsigargin | −1.53 | 205 | 1.47 | 125 | −0.04 | 235 | 220 | 180 | 165 | 200 | 1 |
| Nutlin-3a | −1.62 | 207 | 3.20 | 83 | 14.11 | 107 | 157 | 95 | 145 | 126 | 1 |
| KIN001-055 | −1.97 | 215 | 2.82 | 90 | 15.30 | 102 | 158.5 | 96 | 152.5 | 127.25 | 1 |
| LY317615 | −2.08 | 216 | −0.14 | 194 | 29.12 | 57 | 136.5 | 125.5 | 205 | 131 | 1 |
| THZ-2-49 | −2.10 | 217 | 0.28 | 166 | 46.08 | 15 | 116 | 90.5 | 191.5 | 103.25 | 1 |
| EKB-569 | −2.30 | 222 | 0.83 | 144 | 21.38 | 81 | 151.5 | 112.5 | 183 | 132 | 1 |
| HG-5-88-01 | −2.77 | 232 | 5.46 | 49 | 2.50 | 195 | 213.5 | 122 | 140.5 | 167.75 | 1 |
| BMS-536924 | −3.87 | 241 | 3.90 | 67 | 4.68 | 168 | 204.5 | 117.5 | 154 | 161 | 1 |
| PD-0332991 | −6.01 | 249 | 3.67 | 74 | 3.18 | 190 | 219.5 | 132 | 161.5 | 175.75 | 1 |
| NSC-87877 | 1.03 | 76 | 1.06 | 138 | 8.84 | 135 | 105.5 | 136.5 | 107 | 121 | 0 |
| PLX4720 | 0.98 | 77 | −0.48 | 205 | 5.41 | 164 | 120.5 | 184.5 | 141 | 152.5 | 0 |
| GSK269962A | 0.90 | 79 | 0.94 | 143 | 8.80 | 136 | 107.5 | 139.5 | 111 | 123.5 | 0 |
| MS-275 | 0.85 | 81 | −0.12 | 192 | 6.14 | 153 | 117 | 172.5 | 136.5 | 144.75 | 0 |
| SB590885 | 0.62 | 89 | −0.23 | 198 | 6.15 | 152 | 120.5 | 175 | 143.5 | 147.75 | 0 |
| IOX2 | 0.59 | 90 | 0.38 | 161 | 5.57 | 159 | 124.5 | 160 | 125.5 | 142.25 | 0 |
| Parthenolide | 0.58 | 92 | −0.03 | 185 | 8.47 | 139 | 115.5 | 162 | 138.5 | 138.75 | 0 |
| Imatinib | 0.41 | 101 | 1.27 | 131 | 8.52 | 138 | 119.5 | 134.5 | 116 | 127 | 0 |
| VX-680 | 0.36 | 105 | 0.03 | 179 | 3.44 | 186 | 145.5 | 182.5 | 142 | 164 | 0 |
| GW843682X | 0.35 | 106 | −0.93 | 216 | 3.00 | 191 | 148.5 | 203.5 | 161 | 176 | 0 |
| S-Trityl-L- | 0.34 | 107 | −0.90 | 215 | 4.40 | 173 | 140 | 194 | 161 | 167 | 0 |
| cysteine | | | | | | | | | | | |
| BI-2536 | 0.30 | 108 | −1.48 | 224 | 2.14 | 198 | 153 | 211 | 166 | 182 | 0 |
| YM155 | 0.29 | 109 | −0.12 | 193 | 6.18 | 150 | 129.5 | 171.5 | 151 | 150.5 | 0 |
| Pyrimethamine | 0.28 | 111 | 0.11 | 172 | 5.67 | 158 | 134.5 | 165 | 141.5 | 149.75 | 0 |
| Roscovitine | 0.28 | 112 | −1.51 | 227 | 4.41 | 172 | 142 | 199.5 | 169.5 | 170.75 | 0 |
| GSK-1904529A | 0.19 | 115 | 0.01 | 181 | 3.62 | 182 | 148.5 | 181.5 | 148 | 165 | 0 |
| GNF-2 | 0.17 | 118 | −1.37 | 221 | 4.98 | 166 | 142 | 193.5 | 169.5 | 167.75 | 0 |
| A-443654 | 0.10 | 123 | −2.21 | 235 | 0.69 | 220 | 171.5 | 227.5 | 179 | 199.5 | 0 |
| Paclitaxel | 0.06 | 126 | −2.85 | 239 | 0.51 | 227 | 176.5 | 233 | 182.5 | 204.75 | 0 |
| CMK | −0.01 | 133 | 0.18 | 168 | 5.55 | 161 | 147 | 164.5 | 150.5 | 155.75 | 0 |
| GSK-650394 | −0.07 | 137 | −0.08 | 189 | 3.90 | 177 | 157 | 183 | 163 | 170 | 0 |
| Sorafenib | −0.08 | 138 | 0.04 | 177 | 4.48 | 171 | 154.5 | 174 | 157.5 | 164.25 | 0 |
| CGP-60474 | −0.08 | 139 | −2.09 | 232 | 1.35 | 206 | 172.5 | 219 | 185.5 | 195.75 | 0 |
| Sunitinib | −0.11 | 140 | −0.35 | 203 | 9.34 | 132 | 136 | 167.5 | 171.5 | 151.75 | 0 |
| PHA-665752 | −0.13 | 141 | 1.03 | 140 | 4.62 | 170 | 155.5 | 155 | 140.5 | 155.25 | 0 |
| PLX4720 | −0.15 | 142 | 0.01 | 180 | 5.51 | 162 | 152 | 171 | 161 | 161.5 | 0 |
| (rescreen) | | | | | | | | | | | |
| MG-132 | −0.15 | 144 | −1.78 | 230 | 0.52 | 225 | 184.5 | 227.5 | 187 | 206 | 0 |
| Z-LLNle-CHO | −0.17 | 146 | −2.97 | 241 | 5.95 | 156 | 151 | 198.5 | 193.5 | 174.75 | 0 |
| Bortezomib | −0.23 | 148 | −2.15 | 234 | 0.14 | 233 | 190.5 | 233.5 | 191 | 212 | 0 |
| BMS-509744 | −0.26 | 149 | 0.60 | 153 | 2.90 | 193 | 171 | 173 | 151 | 172 | 0 |
| Salubrinal | −0.26 | 150 | −0.60 | 210 | 9.62 | 129 | 139.5 | 169.5 | 180 | 154.5 | 0 |
| PF-562271 | −0.34 | 153 | 0.13 | 171 | 2.83 | 194 | 173.5 | 182.5 | 162 | 178 | 0 |
| XMD8-92 | −0.45 | 155 | 0.29 | 165 | 6.41 | 148 | 151.5 | 156.5 | 160 | 154 | 0 |
| Vinblastine | −0.45 | 156 | 0.14 | 170 | 7.87 | 140 | 148 | 155 | 163 | 151.5 | 0 |
| CGP-082996 | −0.49 | 159 | −2.78 | 238 | 5.80 | 157 | 158 | 197.5 | 198.5 | 177.75 | 0 |
| CHIR-99021 | −0.52 | 160 | 0.52 | 155 | 6.45 | 146 | 153 | 150.5 | 157.5 | 151.75 | 0 |
| BIRB 0796 | −0.53 | 161 | 0.46 | 158 | 1.04 | 212 | 186.5 | 185 | 159.5 | 185.75 | 0 |
| FTI-277 | −0.58 | 163 | −0.24 | 199 | 3.39 | 188 | 175.5 | 193.5 | 181 | 184.5 | 0 |
| piperlongumine | −0.59 | 164 | 0.00 | 182 | 1.20 | 209 | 186.5 | 195.5 | 173 | 191 | 0 |
| Rapamycin | −0.60 | 165 | −2.56 | 236 | 1.94 | 200 | 182.5 | 218 | 200.5 | 200.25 | 0 |
| GDC0941 | −0.71 | 167 | 0.11 | 173 | 3.85 | 178 | 172.5 | 175.5 | 170 | 174 | 0 |
| JW-7-52-1 | −0.71 | 168 | −3.13 | 243 | 0.40 | 229 | 198.5 | 236 | 205.5 | 217.25 | 0 |
| Pazopanib | −0.72 | 169 | −1.02 | 218 | 0.73 | 217 | 193 | 217.5 | 193.5 | 205.25 | 0 |
| Vinorelbine | −0.74 | 170 | −0.01 | 184 | 3.58 | 183 | 176.5 | 183.5 | 177 | 180 | 0 |
| MK-2206 | −0.81 | 171 | −0.31 | 202 | 3.45 | 185 | 178 | 193.5 | 186.5 | 185.75 | 0 |
| TGX221 | −0.87 | 173 | −5.56 | 249 | −0.27 | 237 | 205 | 243 | 211 | 224 | 0 |
| XMD8-85 | −0.97 | 177 | −0.65 | 212 | 7.87 | 141 | 159 | 176.5 | 194.5 | 167.75 | 0 |
| NVP-TAE684 | −0.99 | 178 | 0.77 | 146 | 0.97 | 214 | 196 | 180 | 162 | 188 | 0 |
| MLN4924 | −1.05 | 180 | 1.02 | 141 | 1.46 | 204 | 192 | 172.5 | 160.5 | 182.25 | 0 |
| JNK-9L | −1.09 | 183 | 1.11 | 136 | 6.17 | 151 | 167 | 143.5 | 159.5 | 155.25 | 0 |
| (5Z)-7- | −1.11 | 186 | −0.15 | 195 | 0.53 | 223 | 204.5 | 209 | 190.5 | 206.75 | 0 |
| Oxozeaenol | | | | | | | | | | | |
| AZD-0530 | −1.16 | 187 | −0.49 | 206 | 0.41 | 228 | 207.5 | 217 | 196.5 | 212.25 | 0 |
| GW 441756 | −1.18 | 189 | 0.64 | 150 | 1.94 | 201 | 195 | 175.5 | 169.5 | 185.25 | 0 |
| WH-4-023 | −1.22 | 191 | −5.52 | 248 | −0.59 | 239 | 215 | 243.5 | 219.5 | 229.25 | 0 |
| Obatoclax | −1.23 | 192 | −0.01 | 183 | 5.45 | 163 | 177.5 | 173 | 187.5 | 175.25 | 0 |
| Mesylate | | | | | | | | | | | |
| Lapatinib | −1.26 | 193 | −3.30 | 244 | −2.24 | 242 | 217.5 | 243 | 218.5 | 230.25 | 0 |
| YK 4-279 | −1.36 | 198 | 0.74 | 147 | 1.40 | 205 | 201.5 | 176 | 172.5 | 188.75 | 0 |
| WZ-1-84 | −1.39 | 199 | −1.68 | 229 | 1.22 | 208 | 203.5 | 218.5 | 214 | 211 | 0 |
| Doxorubicin | −1.45 | 202 | 0.82 | 145 | 2.19 | 197 | 199.5 | 171 | 173.5 | 185.25 | 0 |
| Erlotinib | −1.45 | 203 | −0.56 | 208 | −0.48 | 238 | 220.5 | 223 | 205.5 | 221.75 | 0 |
| A-770041 | −1.58 | 206 | −4.38 | 246 | 0.20 | 232 | 219 | 239 | 226 | 229 | 0 |
| CCT018159 | −1.77 | 208 | 0.25 | 167 | 5.98 | 155 | 181.5 | 161 | 187.5 | 171.25 | 0 |
| NVP-BEZ235 | −1.78 | 209 | −0.44 | 204 | 4.01 | 175 | 192 | 189.5 | 206.5 | 190.75 | 0 |
| RO-3306 | −1.85 | 210 | 0.42 | 160 | 1.29 | 207 | 208.5 | 183.5 | 185 | 196 | 0 |
| Tipifarnib | −1.87 | 211 | −0.55 | 207 | 2.33 | 196 | 203.5 | 201.5 | 209 | 202.5 | 0 |
| Gefitinib | −1.88 | 212 | −0.31 | 201 | 0.99 | 213 | 212.5 | 207 | 206.5 | 209.75 | 0 |
| Dabrafenib | −1.89 | 213 | −1.27 | 219 | 3.36 | 189 | 201 | 204 | 216 | 202.5 | 0 |
| QL-VIII-58 | −1.95 | 214 | −0.03 | 186 | 0.63 | 222 | 218 | 204 | 200 | 211 | 0 |
| Bryostatin 1 | −2.16 | 218 | −1.50 | 226 | 4.64 | 169 | 193.5 | 197.5 | 222 | 195.5 | 0 |
| Cytarabine | −2.20 | 219 | 1.17 | 134 | 0.72 | 218 | 218.5 | 176 | 176.5 | 197.25 | 0 |
| Bosutinib | −2.26 | 220 | −0.72 | 213 | 9.36 | 131 | 175.5 | 172 | 216.5 | 173.75 | 0 |
| AZ628 | −2.27 | 221 | −1.00 | 217 | 0.80 | 216 | 218.5 | 216.5 | 219 | 217.5 | 0 |
| Dasatinib | −2.33 | 223 | −6.21 | 250 | −1.11 | 241 | 232 | 245.5 | 236.5 | 238.75 | 0 |
| Midostaurin | −2.43 | 224 | −0.30 | 200 | 1.17 | 210 | 217 | 205 | 212 | 211 | 0 |
| Elesclomol | −2.43 | 225 | 1.14 | 135 | −4.68 | 244 | 234.5 | 189.5 | 180 | 212 | 0 |
| Epothilone B | −2.44 | 226 | −0.11 | 190 | −0.21 | 236 | 231 | 213 | 208 | 222 | 0 |
| AUY922 | −2.48 | 227 | 0.66 | 149 | 5.15 | 165 | 196 | 157 | 188 | 176.5 | 0 |
| JNJ-26854165 | −2.52 | 228 | 0.36 | 162 | 1.47 | 203 | 215.5 | 182.5 | 195 | 199 | 0 |
| Cetuximab | −2.54 | 229 | −0.11 | 191 | −5.53 | 247 | 238 | 219 | 210 | 228.5 | 0 |
| AKT inhibitor VIII | −2.68 | 230 | −1.95 | 231 | 0.69 | 219 | 224.5 | 225 | 230.5 | 224.75 | 0 |
| Temsirolimus | −2.69 | 231 | −0.06 | 187 | 3.82 | 180 | 205.5 | 183.5 | 209 | 194.5 | 0 |
| AS601245 | −3.03 | 233 | −1.45 | 222 | 2.96 | 192 | 212.5 | 207 | 227.5 | 209.75 | 0 |
| FH535 | −3.03 | 234 | −2.86 | 240 | −2.88 | 243 | 238.5 | 241.5 | 237 | 240 | 0 |
| Embelin | −3.05 | 235 | −0.16 | 196 | 0.21 | 231 | 233 | 213.5 | 215.5 | 223.25 | 0 |
| HG-6-64-1 | −3.51 | 236 | −1.47 | 223 | 3.93 | 176 | 206 | 199.5 | 229.5 | 202.75 | 0 |
| rTRAIL | −3.53 | 237 | 1.06 | 137 | 6.44 | 147 | 192 | 142 | 187 | 167 | 0 |
| Docetaxel | −3.57 | 238 | −0.87 | 214 | −13.95 | 254 | 246 | 234 | 226 | 240 | 0 |
| Afatinib | −3.70 | 239 | −6.43 | 252 | −7.23 | 251 | 245 | 251.5 | 245.5 | 248.25 | 0 |
| Bexarotene | −3.72 | 240 | −0.62 | 211 | 4.39 | 174 | 207 | 192.5 | 225.5 | 199.75 | 0 |
| Bleomycin | −3.89 | 242 | 0.09 | 174 | 0.51 | 226 | 234 | 200 | 208 | 217 | 0 |
| GDC0941 | −4.24 | 243 | −0.60 | 209 | 0.32 | 230 | 236.5 | 219.5 | 226 | 228 | 0 |
| (rescreen) | | | | | | | | | | | |
| CI-1040 | −5.52 | 244 | −1.58 | 228 | 0.13 | 234 | 239 | 231 | 236 | 235 | 0 |
| AZD6482 | −5.75 | 245 | −2.09 | 233 | 3.47 | 184 | 214.5 | 208.5 | 239 | 211.5 | 0 |
| Afatinib | −5.77 | 246 | −3.10 | 242 | −5.06 | 246 | 246 | 244 | 244 | 245 | 0 |
| (rescreen) | | | | | | | | | | | |
| Bleomycin (50 | −5.89 | 247 | 0.61 | 151 | −9.44 | 252 | 249.5 | 201.5 | 199 | 225.5 | 0 |
| uM) | | | | | | | | | | | |
| PD-0325901 | −5.94 | 248 | −4.21 | 245 | −6.84 | 250 | 249 | 247.5 | 246.5 | 248.25 | 0 |
| XAV 939 | −6.51 | 250 | −1.49 | 225 | 1.69 | 202 | 226 | 213.5 | 237.5 | 219.75 | 0 |
| RDEA119 | −9.07 | 251 | −4.88 | 247 | −4.79 | 245 | 248 | 246 | 249 | 247 | 0 |
| 17-AAG | −10.47 | 252 | −13.26 | 255 | −31.83 | 255 | 253.5 | 255 | 253.5 | 254.25 | 0 |
| AZD6244 | −11.76 | 253 | −6.85 | 253 | −5.54 | 248 | 250.5 | 250.5 | 253 | 250.5 | 0 |
| RDEA119 | −12.88 | 254 | −8.88 | 254 | −5.68 | 249 | 251.5 | 251.5 | 254 | 251.5 | 0 |
| (rescreen) | | | | | | | | | | | |
| Trametinib | −15.06 | 255 | −6.25 | 251 | −12.52 | 253 | 254 | 252 | 253 | 253 | 0 |

TABLE 4B
P [symbol] s with sensitivity in SCN, SRBCT or Blood cancer types

| | V1 | | |
|---|---|---|---|
| | (pos = | | |
| | more | | |
| | sensi- | | |
| | tive | | Sensi- |
| | in | | tivity |
| drug_name | SCLC) | rank_V1 | in SCN |
| vorinostat | 0.10 | 1 | 1 |
| VU0238429 | 0.09 | 2 | 1 |
| CUDC-101 | 0.08 | 3 | 1 |
| BX-912 | 0.08 | 4 | 1 |
| oxaliplatin | 0.08 | 5 | 1 |
| alisertib | 0.08 | 6 | 1 |
| salinomycin | 0.08 | 7 | 1 |
| BMS-754807 | 0.08 | 8 | 1 |
| KX2-391 | 0.08 | 9 | 1 |
| D-64131 | 0.08 | 10 | 1 |
| oxyquinoline | 0.08 | 11 | 1 |
| axitinib | 0.07 | 12 | 1 |
| barasertib | 0.07 | 13 | 1 |
| BI-78D3 | 0.07 | 14 | 1 |
| M-344 | 0.07 | 15 | 1 |
| evodiamine | 0.07 | 16 | 1 |
| UNBS-5162 | 0.07 | 17 | 1 |
| vinorelbine | 0.07 | 18 | 1 |
| albendazole | 0.07 | 19 | 1 |
| BI-2536 | 0.07 | 20 | 1 |
| docetaxel | 0.07 | 21 | 1 |
| cetrimonium | 0.07 | 22 | 1 |
| GSK1070916 | 0.07 | 23 | 1 |
| melphalan | 0.07 | 24 | 1 |
| chlormidazole | 0.07 | 25 | 1 |
| fenbendazole | 0.07 | 26 | 1 |
| menadione | 0.07 | 27 | 1 |
| dexrazoxane | 0.07 | 28 | 1 |
| Diphenyl-eneiodonium | 0.07 | 29 | 1 |
| TH-302 | 0.06 | 30 | 1 |
| GW-843682X | 0.06 | 31 | 1 |
| AZ3146 | 0.06 | 32 | 1 |
| EVP4593 | 0.06 | 33 | 1 |
| benzethonium | 0.06 | 34 | 1 |
| PHA-680632 | 0.06 | 35 | 1 |
| Mechlor-ethamine | 0.06 | 36 | 1 |
| trifluridine | 0.06 | 37 | 1 |
| indibulin | 0.06 | 38 | 1 |
| PF-477736 | 0.06 | 39 | 1 |
| scriptaid | 0.06 | 40 | 1 |
| tozasertib | 0.06 | 41 | 1 |
| colchicine | 0.06 | 42 | 1 |
| salinomycin | 0.06 | 43 | 1 |
| SB-225002 | 0.06 | 44 | 1 |
| NH125 | 0.06 | 45 | 1 |
| AVN-944 | 0.06 | 46 | 1 |
| oxaliplatin | 0.06 | 47 | 1 |
| dolastatin-10 | 0.06 | 48 | 1 |
| SC-144 | 0.06 | 49 | 1 |
| LY2183240 | 0.06 | 50 | 1 |
| AR-42 | 0.06 | 51 | 1 |
| quizartinib | 0.06 | 52 | 1 |
| thioguanine | 0.06 | 53 | 1 |
| SU3327 | 0.06 | 54 | 1 |
| methiazole | 0.06 | 55 | 1 |
| resminostat | 0.05 | 56 | 1 |
| doxycycline | 0.05 | 57 | 1 |
| ZM-447439 | 0.05 | 58 | 1 |
| thonzonium | 0.05 | 59 | 1 |
| G-1 | 0.05 | 60 | 1 |
| PF-477736 | 0.05 | 61 | 1 |
| erastin | 0.05 | 62 | 1 |
| narasin | 0.05 | 63 | 1 |
| NSC-663284 | 0.05 | 64 | 1 |
| vincristine | 0.05 | 65 | 1 |
| barasertib-HQPA | 0.05 | 66 | 1 |
| TW-37 | 0.05 | 67 | 1 |
| amonafide | 0.05 | 68 | 1 |
| amsacrine | 0.05 | 69 | 1 |
| JK-184 | 0.05 | 70 | 1 |
| FK-3311 | 0.05 | 71 | 1 |
| trovafloxacin | 0.05 | 72 | 1 |
| amsacrine | 0.05 | 73 | 1 |
| IKK-2-inhibitor-V | 0.05 | 74 | 1 |
| verubulin | 0.05 | 75 | 1 |
| bortezomib | 0.05 | 76 | 1 |
| Hexa-chlorophene | 0.05 | 77 | 1 |
| AZ960 | 0.05 | 78 | 1 |
| taltobulin | 0.05 | 79 | 1 |
| plinabulin | 0.05 | 80 | 1 |
| JIB04 | 0.05 | 81 | 1 |
| Dihydro-artemisinin | 0.05 | 82 | 1 |
| ABT-751 | 0.05 | 83 | 1 |
| Y-320 | 0.05 | 84 | 1 |
| ENMD-2076 | 0.05 | 85 | 1 |
| 2-methoxy-estradiol | 0.05 | 86 | 1 |
| teroxirone | 0.05 | 87 | 1 |
| AMG900 | 0.05 | 88 | 1 |
| AZ20 | 0.05 | 89 | 1 |
| UNC2250 | 0.05 | 90 | 1 |
| 10-deacetyl-baccatin | 0.05 | 91 | 1 |
| JNJ-7706621 | 0.05 | 92 | 1 |
| broxuridine | 0.05 | 93 | 1 |
| CYC116 | 0.05 | 94 | 1 |
| KW-2449 | 0.05 | 95 | 1 |
| tedizolid | 0.05 | 96 | 1 |
| SNS-314 | 0.05 | 97 | 1 |
| polydatin | 0.05 | 98 | 1 |
| NSC-3852 | 0.04 | 99 | 1 |
| gambogic-acid | 0.04 | 100 | 1 |
| PFI-1 | 0.04 | 101 | 1 |
| mubritinib | 0.04 | 102 | 1 |
| litre nesib | 0.04 | 103 | 1 |
| tyrphostin-A9 | 0.04 | 104 | 1 |
| niclosamide | 0.04 | 105 | 1 |
| bardoxolone | 0.04 | 106 | 1 |
| maytansinol-isobutyrate | 0.04 | 107 | 1 |
| apatinib | 0.04 | 108 | 1 |
| chidamide | 0.04 | 109 | 1 |
| chlorhexidine | 0.04 | 110 | 1 |
| KPT-185 | 0.04 | 111 | 1 |
| NMS-873 | 0.04 | 112 | 1 |
| ryuvidine | 0.04 | 113 | 1 |
| dequalinium | 0.04 | 114 | 1 |
| epothilone-d | 0.04 | 115 | 1 |
| danusertib | 0.04 | 116 | 1 |
| KF-38789 | 0.04 | 117 | 1 |
| lasalocid | 0.04 | 118 | 1 |
| foretinib | 0.04 | 119 | 1 |
| OTX015 | 0.04 | 120 | 1 |
| parbendazole | 0.04 | 121 | 1 |
| BAY-11-7082 | 0.04 | 122 | 1 |
| PF-03814735 | 0.04 | 123 | 1 |
| fosbretabulin | 0.04 | 124 | 1 |
| SB-743921 | 0.04 | 125 | 1 |
| sclareol | 0.04 | 126 | 1 |
| digoxin | 0.04 | 127 | 1 |
| BIX-01294 | 0.04 | 128 | 1 |
| PFK-015 | 0.04 | 129 | 1 |
| etoposide | 0.04 | 130 | 1 |
| oxiracetam | 0.04 | 131 | 1 |
| rubitecan | 0.04 | 132 | 1 |
| panobinostat | 0.04 | 133 | 1 |
| crizotinib | 0.04 | 134 | 1 |
| MLN0128 | 0.04 | 135 | 1 |
| NVP-TAE684 | 0.04 | 136 | 1 |
| BAY-11-7085 | 0.04 | 137 | 1 |
| atiprimod | 0.04 | 138 | 1 |
| SB-2343 | 0.04 | 139 | 1 |
| monensin | 0.04 | 140 | 1 |
| lanoconazole | 0.04 | 141 | 1 |
| dalcetrapib | 0.04 | 142 | 1 |
| arctigenin | 0.04 | 143 | 1 |
| vinblastine | 0.04 | 144 | 1 |
| 3′-fluorobenzyl-spiperone | 0.04 | 145 | 1 |
| HA14-1 | 0.04 | 146 | 1 |
| BNTX | 0.04 | 147 | 1 |
| trifluridine | 0.04 | 148 | 1 |
| SB-939 | 0.04 | 149 | 1 |
| chlorambucil | 0.04 | 150 | 1 |
| ICG-001 | 0.04 | 151 | 1 |
| pyrimethamine | 0.04 | 152 | 1 |
| MPI-0479605 | 0.04 | 153 | 1 |
| sarafloxacin | 0.04 | 154 | 1 |
| AT7867 | 0.04 | 155 | 1 |
| anisomycin | 0.04 | 156 | 1 |
| SB-205384 | 0.04 | 157 | 1 |
| RS-17053 | 0.04 | 158 | 1 |
| broxaldine | 0.04 | 159 | 1 |
| AZD3514 | 0.04 | 160 | 1 |
| nortriptyline | 0.04 | 161 | 1 |
| GSK1904529A | 0.04 | 162 | 1 |
| buparlisib | 0.04 | 163 | 1 |
| voreloxin | 0.04 | 164 | 1 |
| curcumin | 0.04 | 165 | 1 |
| phenylmercuric-acetate | 0.04 | 166 | 1 |
| tioguanine | 0.04 | 167 | 1 |
| GDC-0068 | 0.03 | 168 | 1 |
| uprosertib | 0.03 | 169 | 1 |
| k-strophanthidin | 0.03 | 170 | 1 |
| podophyllotoxin | 0.03 | 171 | 1 |
| midostaurin | 0.03 | 172 | 1 |
| harringtoninc | 0.03 | 173 | 1 |
| octenidine | 0.03 | 174 | 1 |
| genz-644282 | 0.03 | 175 | 1 |
| BAY-87-2243 | 0.03 | 176 | 1 |
| YM-155 | 0.03 | 177 | 1 |
| ciclopirox | 0.03 | 178 | 1 |
| ZK-93423 | 0.03 | 179 | 1 |
| irinotecan | 0.03 | 180 | 1 |
| SGI-1027 | 0.03 | 181 | 1 |
| BNC105 | 0.03 | 182 | 1 |
| dovitinib | 0.03 | 183 | 1 |
| palmatine-chloride | 0.03 | 184 | 1 |
| U-18666A | 0.03 | 185 | 1 |
| ledipasvir | 0.03 | 186 | 1 |
| 4-pyrimidine-carbonitrile | 0.03 | 187 | 1 |
| paclitaxel | 0.03 | 188 | 1 |
| adefovir-dipivoxil | 0.03 | 189 | 1 |
| gossypol | 0.03 | 190 | 1 |
| pibenzimol | 0.03 | 191 | 1 |
| ZK811752 | 0.03 | 192 | 1 |
| radezolid | 0.03 | 193 | 1 |
| LY2801653 | 0.03 | 194 | 1 |
| talazoparib | 0.03 | 195 | 1 |
| ingenol-mebutate | 0.03 | 196 | 1 |
| dovitinib | 0.03 | 197 | 1 |
| alvespimycin | 0.03 | 198 | 1 |
| thiomersal | 0.03 | 199 | 1 |
| teniposide | 0.03 | 200 | 1 |
| K-858 | 0.03 | 201 | 1 |
| PR-619 | 0.03 | 202 | 1 |
| oxiperomide | 0.03 | 203 | 1 |
| triapine | 0.03 | 204 | 1 |
| CYT-997 | 0.03 | 205 | 1 |
| parthenolide-(−) | 0.03 | 206 | 1 |
| halofantrine | 0.03 | 207 | 1 |
| Cetyl-pyridinium | 0.03 | 208 | 1 |
| brefeldin-a | 0.03 | 209 | 1 |
| TG-101209 | 0.03 | 210 | 1 |
| clomifene | 0.03 | 211 | 1 |
| TG-101348 | 0.03 | 212 | 1 |
| flubendazole | 0.03 | 213 | 1 |
| mebendazole | 0.03 | 214 | 1 |
| FLI-06 | 0.03 | 215 | 1 |
| AZD8055 | 0.03 | 216 | 1 |
| eltrombopag | 0.03 | 217 | 1 |
| tazemetostat | 0.03 | 218 | 1 |
| dinaciclib | 0.03 | 219 | 1 |
| trichostatin-a | 0.03 | 220 | 1 |
| BMS-265246 | 0.03 | 221 | 1 |
| cyclosporine | 0.03 | 222 | 1 |
| SKI-II | 0.03 | 223 | 1 |
| dolutegravir | 0.03 | 224 | 1 |
| tivantinib | 0.03 | 225 | 1 |
| crizotinib-(S) | 0.03 | 226 | 1 |
| artesunate | 0.03 | 227 | 1 |
| GDC-0349 | 0.03 | 228 | 1 |
| indisulam | 0.03 | 229 | 1 |
| VLX600 | 0.03 | 230 | 1 |
| resiquimod | 0.03 | 231 | 1 |
| anguidine | 0.03 | 232 | 1 |
| CCG-50014 | 0.03 | 233 | 1 |
| amuvatinib | 0.03 | 234 | 1 |
| oxibendazole | 0.03 | 235 | 1 |
| 10-deacetylbaccatin | 0.03 | 236 | 1 |
| oligomycin-a | 0.03 | 237 | 1 |
| nonoxynol-9 | 0.03 | 238 | 1 |
| PIK-75 | 0.03 | 239 | 1 |
| GR-127935 | 0.03 | 240 | 1 |
| MK-8745 | 0.03 | 241 | 1 |
| gemcitabine | 0.03 | 242 | 1 |
| Idarubicin | 0.03 | 243 | 1 |
| mibampator | 0.03 | 244 | 1 |
| Idarubicin | 0.03 | 245 | 1 |
| nuclomedone | 0.03 | 246 | 1 |
| CUDC-907 | 0.03 | 247 | 1 |
| TPCA-1 | 0.03 | 248 | 1 |
| xanomeline | 0.03 | 249 | 1 |
| AZD7762 | 0.03 | 250 | 1 |
| ZM-306416 | 0.03 | 251 | 1 |
| WAY-170523 | 0.03 | 252 | 1 |
| JQ1-(+) | 0.03 | 253 | 1 |
| zaldaride | 0.03 | 254 | 1 |
| mycophenolicacid | 0.03 | 255 | 1 |
| CD-437 | 0.03 | 256 | 1 |
| tipifarnib | 0.03 | 257 | 1 |
| epothilone-a | 0.03 | 258 | 1 |
| palbociclib | 0.03 | 259 | 1 |
| nocodazole | 0.03 | 260 | 1 |
| Pralatrexate | 0.03 | 261 | 1 |
| pilsicainide | 0.03 | 262 | 1 |
| CCT129202 | 0.03 | 263 | 1 |
| OSI-930 | 0.03 | 264 | 1 |
| bitopertin | 0.03 | 265 | 1 |
| betulinic-acid | 0.03 | 266 | 1 |
| PI-103 | 0.03 | 267 | 1 |
| BMS-345541 | 0.03 | 268 | 1 |
| hypoestoxide | 0.03 | 269 | 1 |
| dorzolamide | 0.03 | 270 | 1 |
| 4-(4-fluorobenzoyl)-1- | 0.03 | 271 | 1 |
| (4-phenylbutyl)-piperidine | | | |
| deslanoside | 0.03 | 272 | 1 |
| AZD5363 | 0.03 | 273 | 1 |
| evacetrapib | 0.03 | 274 | 1 |
| PF-562271 | 0.03 | 275 | 1 |
| MNS-(3,4-Methylenedioxy- | 0.03 | 276 | 1 |
| nitrostyrene) | | | |
| nilotinib | 0.03 | 277 | 1 |
| ebastine | 0.03 | 278 | 1 |
| carmofur | 0.03 | 279 | 1 |
| WAY-600 | 0.03 | 280 | 1 |
| SB-216641 | 0.03 | 281 | 1 |
| Oxy-phencyclimine | 0.03 | 282 | 1 |
| ciclesonide | 0.03 | 283 | 1 |
| abemaciclib | 0.03 | 284 | 1 |
| GSK923295 | 0.03 | 285 | 1 |
| bromosporine | 0.03 | 286 | 1 |
| dehydrocholate-acid | 0.03 | 287 | 1 |
| ketoconazole | 0.02 | 288 | 1 |
| 8-bromo-cGMP | 0.02 | 289 | 1 |
| NSC-319726 | 0.02 | 290 | 1 |
| carbachol | 0.02 | 291 | 1 |
| topotecan | 0.02 | 292 | 1 |
| sulconazole | 0.02 | 293 | 1 |
| sorafenib | 0.02 | 294 | 1 |
| E-4031 | 0.02 | 295 | 1 |
| famotidine | 0.02 | 296 | 1 |
| xipamide | 0.02 | 297 | 1 |
| epacadostat | 0.02 | 298 | 1 |
| asenapine | 0.02 | 299 | 1 |
| berberine | 0.02 | 300 | 1 |
| KD-023 | 0.02 | 301 | 1 |
| MLN-8054 | 0.02 | 302 | 1 |
| penfluridol | 0.02 | 303 | 1 |
| tariquidar | 0.02 | 304 | 1 |
| doxycycline | 0.02 | 305 | 1 |
| LGK-974 | 0.02 | 306 | 1 |
| thiamphenicol | 0.02 | 307 | 1 |
| Ro-90-7501 | 0.02 | 308 | 1 |
| clofibrate | 0.02 | 309 | 1 |
| brivaracetam | 0.02 | 310 | 1 |
| grapiprant | 0.02 | 311 | 1 |
| crystal-violet | 0.02 | 312 | 1 |
| PRT062070 | 0.02 | 313 | 1 |
| OTS167 | 0.02 | 314 | 1 |
| vindesine | 0.02 | 315 | 1 |
| RS-504393 | 0.02 | 316 | 1 |
| PTC-209 | 0.02 | 317 | 1 |
| GSK2656157 | 0.02 | 318 | 1 |
| AMG458 | 0.02 | 319 | 1 |
| Bisindolyl-maleimide-ix | 0.02 | 320 | 1 |
| cyproheptadine | 0.02 | 321 | 1 |
| CMPD-1 | 0.02 | 322 | 1 |
| ambazone | 0.02 | 323 | 1 |
| GW-2580 | 0.02 | 324 | 1 |
| sirolimus | 0.02 | 325 | 1 |
| VER-49009 | 0.02 | 326 | 1 |
| LDN-57444 | 0.02 | 327 | 1 |
| PH-797804 | 0.02 | 328 | 1 |
| belinostat | 0.02 | 329 | 1 |
| triptolide | 0.02 | 330 | 1 |
| NSC-23766 | 0.02 | 331 | 1 |
| flunisolide | 0.02 | 332 | 1 |
| EMD-53998 | 0.02 | 333 | 1 |
| OTX015 | 0.02 | 334 | 1 |
| mepacrine | 0.02 | 335 | 1 |
| GR-103691 | 0.02 | 336 | 1 |
| tanshinone-i | 0.02 | 337 | 1 |
| 17-hydroxy-progesterone- | 0.02 | 338 | 1 |
| caproate | | | |
| triptorelin | 0.02 | 339 | 1 |
| ticagrelor | 0.02 | 340 | 1 |
| GSK650394 | 0.02 | 341 | 1 |
| LY2606368 | 0.02 | 342 | 1 |
| enrofloxacin | 0.02 | 343 | 1 |
| cabazitaxel | 0.02 | 344 | 1 |
| WZ-3146 | 0.02 | 345 | 1 |
| obatoclax | 0.02 | 346 | 1 |
| triamterene | 0.02 | 347 | 1 |
| pentamidine | 0.02 | 348 | 1 |
| lomitapide | 0.02 | 349 | 1 |
| ethynodiol-diacetate | 0.02 | 350 | 1 |
| SGI-1776 | 0.02 | 351 | 1 |
| embelin | 0.02 | 352 | 1 |
| skepinone-1 | 0.02 | 353 | 1 |
| sulfabenzamide | 0.02 | 354 | 1 |
| chloropyramine | 0.02 | 355 | 1 |
| iodophenpropit | 0.02 | 356 | 1 |
| domiphen | 0.02 | 357 | 1 |
| daunorubicin | 0.02 | 358 | 1 |
| beclomethasone-dipropionate | 0.02 | 359 | 1 |
| aurora-a-inhibitor-i | 0.02 | 360 | 1 |
| dienogest | 0.02 | 361 | 1 |
| alclometasone-dipropionate | 0.02 | 362 | 1 |
| PF-3845 | 0.02 | 363 | 1 |
| SDZ-21009 | 0.02 | 364 | 1 |
| UNC0631 | 0.02 | 365 | 1 |
| aripiprazole | 0.02 | 366 | 1 |
| lappaconite | 0.02 | 367 | 1 |
| volasertib | 0.02 | 368 | 1 |
| Y-27632 | 0.02 | 369 | 1 |
| HMN-214 | 0.02 | 370 | 1 |
| romidepsin | 0.02 | 371 | 1 |
| Medroxy-progesterone-acetate | 0.02 | 372 | 1 |
| mericitabine | 0.02 | 373 | 1 |
| MK-1775 | 0.02 | 374 | 1 |
| NMS-1286937 | 0.02 | 375 | 1 |
| benzonatate | 0.02 | 376 | 1 |
| SC-560 | 0.02 | 377 | 1 |
| KD025 | 0.02 | 378 | 1 |
| cimetidine | 0.02 | 379 | 1 |
| ASC-J9 | 0.02 | 380 | 1 |
| calcitriol | 0.02 | 381 | 1 |
| SNAP-94847 | 0.02 | 382 | 1 |
| SCH-900776 | 0.02 | 383 | 1 |
| niguldipine-(S)-(+) | 0.02 | 384 | 1 |
| dapivirine | 0.02 | 385 | 1 |
| SR-27897 | 0.02 | 386 | 1 |
| olaparib | 0.02 | 387 | 1 |
| GSK1292263 | 0.02 | 388 | 1 |
| Homoharringtonine | 0.02 | 389 | 1 |
| GSK2110183 | 0.02 | 390 | 1 |
| PETCM | 0.02 | 391 | 1 |
| acitretin | 0.02 | 392 | 1 |
| I-BET-762 | 0.02 | 393 | 1 |
| ACY-1215 | 0.02 | 394 | 1 |
| KP-1212 | 0.02 | 395 | 1 |
| retapamulin | 0.02 | 396 | 1 |
| UNC0737 | 0.02 | 397 | 1 |
| algestone-acetophenide | 0.02 | 398 | 1 |
| acesulfame-potassium | 0.02 | 399 | 1 |
| nobiletin | 0.02 | 400 | 1 |
| ispinesib | 0.02 | 401 | 1 |
| amitriptyline | 0.02 | 402 | 1 |
| eprinomectin | 0.02 | 403 | 1 |
| eperezolid | 0.02 | 404 | 1 |
| meclizine | 0.02 | 405 | 1 |
| IKK-16 | 0.02 | 406 | 1 |
| metatinib | 0.02 | 407 | 1 |
| fenoldopam | 0.02 | 408 | 1 |
| SB-228357 | 0.02 | 409 | 1 |
| ivermectin | 0.02 | 410 | 1 |
| tranylcypromine | 0.02 | 411 | 1 |
| ibuproxam | 0.02 | 412 | 1 |
| Ro-19-4605 | 0.02 | 413 | 1 |
| cepharanthine | 0.02 | 414 | 1 |
| MK-8245 | 0.02 | 415 | 1 |
| MBX-2982 | 0.02 | 416 | 1 |
| maltobionic-acid | 0.02 | 417 | 1 |
| tasisulam | 0.02 | 418 | 1 |
| tacedinaline | 0.02 | 419 | 1 |
| ZK-200775 | 0.02 | 420 | 1 |
| clofazimine | 0.02 | 421 | 1 |
| SB-590885 | 0.02 | 422 | 1 |
| CI-844 | 0.02 | 423 | 1 |
| filanesib | 0.02 | 424 | 1 |
| amiloride | 0.02 | 425 | 1 |
| CI-966 | 0.02 | 426 | 1 |
| temazepam | 0.02 | 427 | 1 |
| GNF-2 | 0.02 | 428 | 1 |
| bentazepam | 0.02 | 429 | 1 |
| OLDA | 0.02 | 430 | 1 |
| RAF265 | 0.02 | 431 | 1 |
| Benzyldimethylhexadecylammonium | 0.02 | 432 | 1 |
| SN-38 | 0.02 | 433 | 1 |
| carmustine | 0.02 | 434 | 1 |
| pyroxamide | 0.02 | 435 | 1 |
| oprozomib | 0.02 | 436 | 1 |
| MK-2206 | 0.02 | 437 | 1 |
| IBC-293 | 0.02 | 438 | 1 |
| tofacitinib | 0.02 | 439 | 1 |
| BIIB021 | 0.02 | 440 | 1 |
| methotrexate | 0.02 | 441 | 1 |
| RKI-1447 | 0.02 | 442 | 1 |
| fingolimod | 0.02 | 443 | 1 |
| chlorprothixene | 0.02 | 444 | 1 |
| AT-9283 | 0.02 | 445 | 1 |
| flumatinib | 0.02 | 446 | 1 |
| geni stein | 0.02 | 447 | 1 |
| gastrodin | 0.02 | 448 | 1 |
| NVP-BHG712 | 0.02 | 449 | 1 |
| K-Ras(G12C)-inhibitor-6 | 0.02 | 450 | 1 |
| L-701324 | 0.02 | 451 | 1 |
| NSC-697923 | 0.02 | 452 | 1 |
| nexturastat-a | 0.02 | 453 | 1 |
| megestrol-acetate | 0.02 | 454 | 1 |
| ixazomib | 0.02 | 455 | 1 |
| darifenacin | 0.02 | 456 | 1 |
| azacitidine | 0.02 | 457 | 1 |
| ONC201 | 0.02 | 458 | 1 |
| azaperone | 0.02 | 459 | 1 |
| rabeprazole | 0.02 | 460 | 1 |
| boceprevir | 0.02 | 461 | 1 |
| trimethoprim | 0.02 | 462 | 1 |
| prilocaine | 0.02 | 463 | 1 |
| SB-505124 | 0.02 | 464 | 1 |
| STA-5326 | 0.02 | 465 | 1 |
| PF-573228 | 0.02 | 466 | 1 |
| AZD7545 | 0.02 | 467 | 1 |
| stepronin | 0.02 | 468 | 1 |
| niraparib | 0.02 | 469 | 1 |
| telaprevir | 0.02 | 470 | 1 |
| vinflunine | 0.02 | 471 | 1 |
| PF-04136309 | 0.02 | 472 | 1 |
| amorolfine | 0.02 | 473 | 1 |
| AMG-9810 | 0.02 | 474 | 1 |
| PSB-1115 | 0.02 | 475 | 1 |
| basimglurant | 0.02 | 476 | 1 |
| medrysone | 0.02 | 477 | 1 |
| nelfinavir | 0.02 | 478 | 1 |
| 1-phenylbiguanide | 0.02 | 479 | 1 |
| levobunolol | 0.02 | 480 | 1 |
| echinomycin | 0.02 | 481 | 1 |
| AZD1480 | 0.02 | 482 | 1 |
| fluspirilene | 0.02 | 483 | 1 |
| Carboxyamidotriazole | 0.02 | 484 | 1 |
| fluorometholone | 0.02 | 485 | 1 |
| GDC-0980 | 0.02 | 486 | 1 |
| tetramethylthiuram-monosulfide | 0.02 | 487 | 1 |
| DPPE | 0.02 | 488 | 1 |
| alfacalcidol | 0.02 | 489 | 1 |
| pazopanib | 0.02 | 490 | 1 |
| valrubicin | 0.02 | 491 | 1 |
| loteprednol | 0.02 | 492 | 1 |
| CX-5461 | 0.02 | 493 | 1 |
| etanidazole | 0.02 | 494 | 1 |
| cytochlor | 0.02 | 495 | 1 |
| AY-9944 | 0.02 | 496 | 1 |
| sildenafil | 0.02 | 497 | 1 |
| methacycline | 0.02 | 498 | 1 |
| ribavirin | 0.02 | 499 | 1 |
| IOX2 | 0.02 | 500 | 1 |
| CCT128930 | 0.02 | 501 | 1 |
| tasuldine | 0.02 | 502 | 1 |
| ritonavir | 0.02 | 503 | 1 |
| 4E1RCat | 0.02 | 504 | 1 |
| pregnenolone-succinate | 0.02 | 505 | 1 |
| beta-lapachone | 0.02 | 506 | 1 |
| dehydrocory [symbol] e | 0.02 | 507 | 1 |
| thioridazine | 0.02 | 508 | 1 |
| aniracetam | 0.02 | 509 | 1 |
| LY2228820 | 0.02 | 510 | 1 |
| mocetinostat | 0.02 | 511 | 1 |
| methylprednisolone-sodium-succinate | 0.02 | 512 | 1 |
| WIN-64338 | 0.02 | 513 | 1 |
| SDZ-WAG-994 | 0.02 | 514 | 1 |
| bevantolol | 0.02 | 515 | 1 |
| haloperidol | 0.02 | 516 | 1 |
| L-701252 | 0.02 | 517 | 1 |
| CP-339818 | 0.02 | 518 | 1 |
| deferasirox | 0.02 | 519 | 1 |
| rutaecarpine | 0.02 | 520 | 1 |
| BI-D1870 | 0.02 | 521 | 1 |
| ginsenoside-rg3 | 0.02 | 522 | 1 |
| VE-821 | 0.02 | 523 | 1 |
| zolantidine | 0.02 | 524 | 1 |
| clobetasol-propionate | 0.02 | 525 | 1 |
| NOV-002 | 0.02 | 526 | 1 |
| LY411575 | 0.02 | 527 | 1 |
| 5′-Chloro-5′-deoxy-ENBA-(+/−) | 0.02 | 528 | 1 |
| aphidicolin | 0.02 | 529 | 1 |
| triclocarban | 0.02 | 530 | 1 |
| XL-147 | 0.02 | 531 | 1 |
| PCI-24781 | 0.02 | 532 | 1 |
| mycophenolate-mofetil | 0.02 | 533 | 1 |
| NGB-2904 | 0.02 | 534 | 1 |
| PI-828 | 0.02 | 535 | 1 |
| clozapine | 0.02 | 536 | 1 |
| fexinidazole | 0.02 | 537 | 1 |
| NPC-15199 | 0.02 | 538 | 1 |
| methotrexate | 0.02 | 539 | 1 |
| lopinavir | 0.02 | 540 | 1 |
| ICI-118,551 | 0.02 | 541 | 1 |
| serdemetan | 0.02 | 542 | 1 |
| sirolimus | 0.02 | 543 | 1 |
| CH223191 | 0.02 | 544 | 1 |
| buparvaquone | 0.02 | 545 | 1 |
| RGFP966 | 0.02 | 546 | 1 |
| esaprazole | 0.02 | 547 | 1 |
| costunolide | 0.02 | 548 | 1 |
| bromperidol | 0.02 | 549 | 1 |
| lapatinib | 0.02 | 550 | 1 |
| PKI-179 | 0.02 | 551 | 1 |
| ABT-737 | 0.02 | 552 | 1 |
| amisulpride | 0.02 | 553 | 1 |
| KI-16425 | 0.02 | 554 | 1 |
| andrographolide | 0.02 | 555 | 1 |
| A-366 | 0.02 | 556 | 1 |
| CRT0044876 | 0.02 | 557 | 1 |
| L-741742 | 0.02 | 558 | 1 |
| P276-00 | 0.02 | 559 | 1 |
| efloxate | 0.02 | 560 | 1 |
| YM-155 | 0.02 | 561 | 1 |
| T-0901317 | 0.02 | 562 | 1 |
| propafenone | 0.02 | 563 | 1 |
| merimepodib | 0.02 | 564 | 1 |
| napabucasin | 0.02 | 565 | 1 |
| SB-431542 | 0.02 | 566 | 1 |
| allylisothiocyanate | 0.02 | 567 | 1 |
| oridonin | 0.02 | 568 | 1 |
| azathioprine | 0.02 | 569 | 1 |
| KPT-276 | 0.02 | 570 | 1 |
| limonin | 0.02 | 571 | 1 |
| piperazinedione | 0.02 | 572 | 1 |
| GBR-12783 | 0.02 | 573 | 1 |
| 2-fluoro-2-deoxy-D-galactose | 0.02 | 574 | 1 |
| furosemide | 0.02 | 575 | 1 |
| diflorasone-diacetate | 0.02 | 576 | 1 |
| LFM-A13 | 0.02 | 577 | 1 |
| idoxuridine | 0.02 | 578 | 1 |
| dexlansoprazole | 0.02 | 579 | 1 |
| cabozantinib | 0.02 | 580 | 1 |
| combretastatin-A-4 | 0.01 | 581 | 1 |
| cidofovir | 0.01 | 582 | 1 |
| praziquantel | 0.01 | 583 | 1 |
| I-BET151 | 0.01 | 584 | 1 |
| megestrol-acetate | 0.01 | 585 | 1 |
| iopamidol | 0.01 | 586 | 1 |
| thiostrepton | 0.01 | 587 | 1 |
| nisoxetine | 0.01 | 588 | 1 |
| olopatadine | 0.01 | 589 | 1 |
| progesterone | 0.01 | 590 | 1 |
| nitisinone | 0.01 | 591 | 1 |
| tubocurarine | 0.01 | 592 | 1 |
| letosteine | 0.01 | 593 | 1 |
| cebranopadol | 0.01 | 594 | 1 |
| RG2833 | 0.01 | 595 | 1 |
| azithromycin | 0.01 | 596 | 1 |
| taladegib | 0.01 | 597 | 1 |
| TMPH | 0.01 | 598 | 1 |
| mifepristone | 0.01 | 599 | 1 |
| buspirone | 0.01 | 600 | 1 |
| valnemulin | 0.01 | 601 | 1 |
| doxycycline | 0.01 | 602 | 1 |
| cholecalciferol | 0.01 | 603 | 1 |
| FK-962 | 0.01 | 604 | 1 |
| fusidic-acid | 0.01 | 605 | 1 |
| florfenicol | 0.01 | 606 | 1 |
| SCS | 0.01 | 607 | 1 |
| velneperit | 0.01 | 608 | 1 |
| hydrocortisone | 0.01 | 609 | 1 |
| semagacestat | 0.01 | 610 | 1 |
| desonide | 0.01 | 611 | 1 |
| MK-0893 | 0.01 | 612 | 1 |
| entacapone | 0.01 | 613 | 1 |
| venetoclax | 0.01 | 614 | 1 |
| zalcitabine | 0.01 | 615 | 1 |
| T-0070907 | 0.01 | 616 | 1 |
| compound-401 | 0.01 | 617 | 1 |
| CTS-1027 | 0.01 | 618 | 1 |
| cephalosporin-c-zn | 0.01 | 619 | 1 |
| aminoguanidine | 0.01 | 620 | 1 |
| BMY-7378 | 0.01 | 621 | 1 |
| ethaverine | 0.01 | 622 | 1 |
| NBQX | 0.01 | 623 | 1 |
| hydrocortisone-valerate | 0.01 | 624 | 1 |
| AMG458 | 0.01 | 625 | 1 |
| NECA | 0.01 | 626 | 1 |
| danirixin | 0.01 | 627 | 1 |
| dipraglurant | 0.01 | 628 | 1 |
| AGN-192403 | 0.01 | 629 | 1 |
| CS-917 | 0.01 | 630 | 1 |
| LY2140023 | 0.01 | 631 | 1 |
| clomipramine | 0.01 | 632 | 1 |
| NMDA | 0.01 | 633 | 1 |
| FR-180204 | 0.01 | 634 | 1 |
| sunitinib | 0.01 | 635 | 1 |
| R-428 | 0.01 | 636 | 1 |
| corticosterone | 0.01 | 637 | 1 |
| homatropine | 0.01 | 638 | 1 |
| arbutin | 0.01 | 639 | 1 |
| DBeQ | 0.01 | 640 | 1 |
| S-14506 | 0.01 | 641 | 1 |
| itraconazole | 0.01 | 642 | 1 |
| meclocycline-sulfosalicylate | 0.01 | 643 | 1 |
| 3-carboxy-4- | 0.01 | 644 | 1 |
| hydroxyphenylglycine-(S) | | | |
| erteberel | 0.01 | 645 | 1 |
| dofequidar | 0.01 | 646 | 1 |
| miglustat | 0.01 | 647 | 1 |
| everolimus | 0.01 | 648 | 1 |
| SB-939 | 0.01 | 649 | 1 |
| hygromycin-b | 0.01 | 650 | 1 |
| ziprasidone | 0.01 | 651 | 1 |
| SKLB1002 | 0.01 | 652 | 1 |
| baicalin | 0.01 | 653 | 1 |
| CO-101244 | 0.01 | 654 | 1 |
| darapladib | 0.01 | 655 | 1 |
| OG-L002 | 0.01 | 656 | 1 |
| danusertib | 0.01 | 657 | 1 |
| ASA-404 | 0.01 | 658 | 1 |
| tanespimycin | 0.01 | 659 | 1 |
| mercaptopurine | 0.01 | 660 | 1 |
| dexniguldipine | 0.01 | 661 | 1 |
| SB-203186 | 0.01 | 662 | 1 |
| xaliproden | 0.01 | 663 | 1 |
| mibefradil | 0.01 | 664 | 1 |
| elbasvir | 0.01 | 665 | 1 |
| SANT-2 | 0.01 | 666 | 1 |
| flufenamic-acid | 0.01 | 667 | 1 |
| Hydroxylprogesterone | 0.01 | 668 | 1 |
| pranidipine | 0.01 | 669 | 1 |
| prednisolone-hemisuccinate | 0.01 | 670 | 1 |
| deptropine | 0.01 | 671 | 1 |
| NVP-BSK805 | 0.01 | 672 | 1 |
| avasimibe | 0.01 | 673 | 1 |
| apabetalone | 0.01 | 674 | 1 |
| oxybuprocaine | 0.01 | 675 | 1 |
| SR-33805 | 0.01 | 676 | 1 |
| NTNCB | 0.01 | 677 | 1 |
| IPI-145 | 0.01 | 678 | 1 |
| pentylenetetrazol | 0.01 | 679 | 1 |
| enasidenib | 0.01 | 680 | 1 |
| arglabin | 0.01 | 681 | 1 |
| Diethylcarbamazine | 0.01 | 682 | 1 |
| SU-11274 | 0.01 | 683 | 1 |
| dacarbazine | 0.01 | 684 | 1 |
| flumethasone | 0.01 | 685 | 1 |
| 10058-F4 | 0.01 | 686 | 1 |
| doxycycline | 0.01 | 687 | 1 |
| gliclazide | 0.01 | 688 | 1 |
| betamethasone | 0.01 | 689 | 1 |
| alexidine | 0.01 | 690 | 1 |
| floctafenine | 0.01 | 691 | 1 |
| riluzole | 0.01 | 692 | 1 |
| BAY-K-8644-(+/−) | 0.01 | 693 | 1 |
| UB-165 | 0.01 | 694 | 1 |
| talmapimod | 0.01 | 695 | 1 |
| etodolac | 0.01 | 696 | 1 |
| AQ-RA741 | 0.01 | 697 | 1 |
| sertindole | 0.01 | 698 | 1 |
| oritavancin | 0.01 | 699 | 1 |
| isoflurane | 0.01 | 700 | 1 |
| ocinaplon | 0.01 | 701 | 1 |
| SB-525334 | 0.01 | 702 | 1 |
| amprenavir | 0.01 | 703 | 1 |
| bafetinib | 0.01 | 704 | 1 |
| thalidomide | 0.01 | 705 | 1 |
| tenovin-6 | 0.01 | 706 | 1 |
| levodropropizine | 0.01 | 707 | 1 |
| droxinostat | 0.01 | 708 | 1 |
| xenalipin | 0.01 | 709 | 1 |
| butylphthalide | 0.01 | 710 | 1 |
| marbofloxacin | 0.01 | 711 | 1 |
| DAPT | 0.01 | 712 | 1 |
| mazindol | 0.01 | 713 | 1 |
| vorapaxar | 0.01 | 714 | 1 |
| benurestat | 0.01 | 715 | 1 |
| cirazoline | 0.01 | 716 | 1 |
| BRL-15572 | 0.01 | 717 | 1 |
| glutamine-(1) | 0.01 | 718 | 1 |
| inosine | 0.01 | 719 | 1 |
| 3-alpha-Hydroxy-5-beta- | 0.01 | 720 | 1 |
| androstan-17-one | | | |
| chlormadinone-acetate | 0.01 | 721 | 1 |
| AMG-337 | 0.01 | 722 | 1 |
| daunorubicin | 0.01 | 723 | 1 |
| chloramphenicol | 0.01 | 724 | 1 |
| D-4476 | 0.01 | 725 | 1 |
| prolylleucylglycinamide | 0.01 | 726 | 1 |
| EHop-016 | 0.01 | 727 | 1 |
| amodiaquine | 0.01 | 728 | 1 |
| remoxipride | 0.01 | 729 | 1 |
| drospirenone | 0.01 | 730 | 1 |
| sulfanitran | 0.01 | 731 | 1 |
| WAY-100635 | 0.01 | 732 | 1 |
| pitavastatin | 0.01 | 733 | 1 |
| Ro-106-9920 | 0.01 | 734 | 1 |
| cinoxacin | 0.01 | 735 | 1 |
| givinostat | 0.01 | 736 | 1 |
| moroxydine | 0.01 | 737 | 1 |
| haloprogin | 0.01 | 738 | 1 |
| telotristat-ethyl | 0.01 | 739 | 1 |
| todralazine | 0.01 | 740 | 1 |
| oleoylethanolamide | 0.01 | 741 | 1 |
| cefixime | 0.01 | 742 | 1 |
| H3-MECA | 0.01 | 743 | 1 |
| TG100-115 | 0.01 | 744 | 1 |
| AG-14361 | 0.01 | 745 | 1 |
| VX-702 | 0.01 | 746 | 1 |
| tanaproget | 0.01 | 747 | 1 |
| solithromycin | 0.01 | 748 | 1 |
| rigosertib | 0.01 | 749 | 1 |
| cefepime | 0.01 | 750 | 1 |
| pipamperone | 0.01 | 751 | 1 |
| grazoprevir | 0.01 | 752 | 1 |
| betahistine | 0.01 | 753 | 1 |
| PNU-74654 | 0.01 | 754 | 1 |
| taxifolin | 0.01 | 755 | 1 |
| mifobate | 0.01 | 756 | 1 |
| mirodenafil | 0.01 | 757 | 1 |
| disulfiram | 0.01 | 758 | 1 |
| piromidic-acid | 0.01 | 759 | 1 |
| nefiracetam | 0.01 | 760 | 1 |
| teniposide | 0.01 | 761 | 1 |
| darusentan | 0.01 | 762 | 1 |
| cyclovalone | 0.01 | 763 | 1 |
| GDC-0834 | 0.01 | 764 | 1 |
| dydrogesterone | 0.01 | 765 | 1 |
| escitalopram | 0.01 | 766 | 1 |
| nemorubicin | 0.01 | 767 | 1 |
| temsirolimus | 0.01 | 768 | 1 |
| cefditoren-pivoxil | 0.01 | 769 | 1 |
| efinaconazole | 0.01 | 770 | 1 |
| mitomycin-c | 0.01 | 771 | 1 |
| perifosine | 0.01 | 772 | 1 |
| LY2608204 | 0.01 | 773 | 1 |
| droxidopa | 0.01 | 774 | 1 |
| chlorthalidone | 0.01 | 775 | 1 |
| PHA-665752 | 0.01 | 776 | 1 |
| indatraline | 0.01 | 777 | 1 |
| L-670596 | 0.01 | 778 | 1 |
| mozavaptan | 0.01 | 779 | 1 |
| TAS-103 | 0.01 | 780 | 1 |
| PAC-1 | 0.01 | 781 | 1 |
| brompheniramine | 0.01 | 782 | 1 |
| mestinon | 0.01 | 783 | 1 |
| acetylcholine | 0.01 | 784 | 1 |
| mifepristone | 0.01 | 785 | 1 |
| ramosetron | 0.01 | 786 | 1 |
| pruvanserin | 0.01 | 787 | 1 |
| PLX-4720 | 0.01 | 788 | 1 |
| idazoxan | 0.01 | 789 | 1 |
| idelalisib | 0.01 | 790 | 1 |
| YM-58483 | 0.01 | 791 | 1 |
| reboxetine | 0.01 | 792 | 1 |
| dimethyl-fumarate | 0.01 | 793 | 1 |
| prednisolone-acetate | 0.01 | 794 | 1 |
| pimecrolimus | 0.01 | 795 | 1 |
| spaglumic-acid | 0.01 | 796 | 1 |
| paliperidone | 0.01 | 797 | 1 |
| LB42708 | 0.01 | 798 | 1 |
| LY2334737 | 0.01 | 799 | 1 |
| VE-822 | 0.01 | 800 | 1 |
| L-165041 | 0.01 | 801 | 1 |
| altretamine | 0.01 | 802 | 1 |
| orbifloxacin | 0.01 | 803 | 1 |
| mavoglurant | 0.01 | 804 | 1 |
| altrenogest | 0.01 | 805 | 1 |
| fagomine | 0.01 | 806 | 1 |
| 2-oxoglutaric-acid | 0.01 | 807 | 1 |
| PRT062607 | 0.01 | 808 | 1 |
| NNC-05-2090 | 0.01 | 809 | 1 |
| diclazuril | 0.01 | 810 | 1 |
| lanatoside-c | 0.01 | 811 | 1 |
| tivozanib | 0.01 | 812 | 1 |
| erythromycin | 0.01 | 813 | 1 |
| rheochrysidin | 0.01 | 814 | 1 |
| ACDPP | 0.01 | 815 | 1 |
| nifuroxazide | 0.01 | 816 | 1 |
| AZD1208 | 0.01 | 817 | 1 |
| PF-05212384 | 0.01 | 818 | 1 |
| toloxatone | 0.01 | 819 | 1 |
| MGCD-265 | 0.01 | 820 | 1 |
| kifunensine | 0.01 | 821 | 1 |
| cyclocytidine | 0.01 | 822 | 1 |
| aliskiren-hemifumarate | 0.01 | 823 | 1 |
| flavoxate | 0.01 | 824 | 1 |
| losmapimod | 0.01 | 825 | 1 |
| ergonovine | 0.01 | 826 | 1 |
| metrizamide | 0.01 | 827 | 1 |
| trimebutine | 0.01 | 828 | 1 |
| chloramphenicol-palmitate | 0.01 | 829 | 1 |
| golvatinib | 0.01 | 830 | 1 |
| afalanine | 0.01 | 831 | 1 |
| trimethoquinol | 0.01 | 832 | 1 |
| rotigotine | 0.01 | 833 | 1 |
| SGC-0946 | 0.01 | 834 | 1 |
| exifone | 0.01 | 835 | 1 |
| C646 | 0.01 | 836 | 1 |
| SB-222200 | 0.01 | 837 | 1 |
| SRT1720 | 0.01 | 838 | 1 |
| loxapine | 0.01 | 839 | 1 |
| misoprostol | 0.01 | 840 | 1 |
| N6-cyclopentyladenosine | 0.01 | 841 | 1 |
| tecovirimat | 0.01 | 842 | 1 |
| arbidol | 0.01 | 843 | 1 |
| butamben | 0.01 | 844 | 1 |
| CITCO | 0.01 | 845 | 1 |
| linezolid | 0.01 | 846 | 1 |
| brivudine | 0.01 | 847 | 1 |
| piricapiron | 0.01 | 848 | 1 |
| chlorprothixene | 0.01 | 849 | 1 |
| rimonabant | 0.01 | 850 | 1 |
| GSK-J4 | 0.01 | 851 | 1 |
| balofloxacin | 0.01 | 852 | 1 |
| homatropine-methylbromide | 0.01 | 853 | 1 |
| penbutolol | 0.01 | 854 | 1 |
| afatinib | 0.01 | 855 | 1 |
| anecortave-acetate | 0.01 | 856 | 1 |
| PNU-142633 | 0.01 | 857 | 1 |
| lofemizole | 0.01 | 858 | 1 |
| BH3N4096 | 0.01 | 859 | 1 |
| terfenadine | 0.01 | 860 | 1 |
| cyclizine | 0.01 | 861 | 1 |
| ticlopidine | 0.01 | 862 | 1 |
| PD-98059 | 0.01 | 863 | 1 |
| IDO5L | 0.01 | 864 | 1 |
| warfarin | 0.01 | 865 | 1 |
| NSI-189 | 0.01 | 866 | 1 |
| methylergometrine | 0.01 | 867 | 1 |
| iodoquinol | 0.01 | 868 | 1 |
| momiflumate | 0.01 | 869 | 1 |
| ibrolipim | 0.01 | 870 | 1 |
| CF102 | 0.01 | 871 | 1 |
| desmopressin-acetate | 0.01 | 872 | 1 |
| naringin-dihydrochalcone | 0.01 | 873 | 1 |
| balaglitazone | 0.01 | 874 | 1 |
| amfepramone | 0.01 | 875 | 1 |
| sulbentine | 0.01 | 876 | 1 |
| quinidine | 0.01 | 877 | 1 |
| ecamsule-triethanolamine | 0.01 | 878 | 1 |
| olmesartan-medoxomil | 0.01 | 879 | 1 |
| mebicar | 0.01 | 880 | 1 |
| ravuconazole | 0.01 | 881 | 1 |
| dacomitinib | 0.01 | 882 | 1 |
| idoxuridine | 0.01 | 883 | 1 |
| GW-9508 | 0.01 | 884 | 1 |
| NKP-1339 | 0.01 | 885 | 1 |
| tetrindole | 0.01 | 886 | 1 |
| CTEP | 0.01 | 887 | 1 |
| IWP-L6 | 0.01 | 888 | 1 |
| WZ-4002 | 0.01 | 889 | 1 |
| hexaminolevulinate | 0.01 | 890 | 1 |
| darglitazone | 0.01 | 891 | 1 |
| ORE1001 | 0.01 | 892 | 1 |
| medorinone | 0.01 | 893 | 1 |
| OMDM-2 | 0.01 | 894 | 1 |
| ilomastat | 0.01 | 895 | 1 |
| GZD824 | 0.01 | 896 | 1 |
| tyrphostin-AG-835 | 0.01 | 897 | 1 |
| etacrynic-acid | 0.01 | 898 | 1 |
| semapimod | 0.01 | 899 | 1 |
| oridonin | 0.01 | 900 | 1 |
| digitoxigenin | 0.01 | 901 | 1 |
| cardiogenol-c | 0.01 | 902 | 1 |
| deforolimus | 0.01 | 903 | 1 |
| LY2874455 | 0.01 | 904 | 1 |
| CNX-774 | 0.01 | 905 | 1 |
| cefpirome | 0.01 | 906 | 1 |
| fluoromethylcholine | 0.01 | 907 | 1 |
| caramiphen | 0.01 | 908 | 1 |
| zimelidine | 0.01 | 909 | potential |
| CFM-2 | 0.01 | 910 | potential |
| cathepsin-inhibitor-1 | 0.01 | 911 | potential |
| BIX-01294 | 0.01 | 912 | potential |
| leteprinim | 0.01 | 913 | potential |
| diosgenin | 0.01 | 914 | potential |
| tolperisone | 0.01 | 915 | potential |
| NU-7441 | 0.01 | 916 | potential |
| etonogestrel | 0.01 | 917 | potential |
| magnolol | 0.01 | 918 | potential |
| vidarabine | 0.01 | 919 | potential |
| talc | 0.01 | 920 | potential |
| piperazine | 0.01 | 921 | potential |
| gemcadiol | 0.01 | 922 | potential |
| homatropine | 0.01 | 923 | potential |
| SB-203580 | 0.01 | 924 | potential |
| dihydroergocristine | 0.01 | 925 | potential |
| rifampin | 0.01 | 926 | potential |
| tranilast | 0.01 | 927 | potential |
| ZM-336372 | 0.01 | 928 | potential |
| cefditoren-pivoxil | 0.01 | 929 | potential |
| seocalcitol | 0.01 | 930 | potential |
| doramectin | 0.01 | 931 | potential |
| ataluren | 0.01 | 932 | potential |
| isobutyramide | 0.01 | 933 | potential |
| domperidone | 0.01 | 934 | potential |
| choline | 0.01 | 935 | potential |
| mupirocin | 0.01 | 936 | potential |
| besifloxacin | 0.01 | 937 | potential |
| indirubin-3-monoxime | 0.01 | 938 | potential |
| A61603 | 0.01 | 939 | potential |
| 2-hydroxyethyl-salicylate | 0.01 | 940 | potential |
| riboflavin | 0.01 | 941 | potential |
| GSK2636771 | 0.01 | 942 | potential |
| UNC0642 | 0.01 | 943 | potential |
| rilpivirine | 0.01 | 944 | potential |
| SYM-2206 | 0.01 | 945 | potential |
| NVP-AEW541 | 0.01 | 946 | potential |
| fluticasone-propionate | 0.01 | 947 | potential |
| JNJ-26481585 | 0.01 | 948 | potential |
| SB-408124 | 0.01 | 949 | potential |
| aprindine | 0.01 | 950 | potential |
| trometamol | 0.01 | 951 | potential |
| minoxidil | 0.01 | 952 | potential |
| GPla | 0.01 | 953 | potential |
| cyt387 | 0.01 | 954 | potential |
| ursolic-acid | 0.01 | 955 | potential |
| PF-03758309 | 0.01 | 956 | potential |
| PA-824 | 0.01 | 957 | potential |
| tegobuvir | 0.01 | 958 | potential |
| 2-CMDO | 0.01 | 959 | potential |
| nemonapride | 0.01 | 960 | potential |
| trospium | 0.01 | 961 | potential |
| oncrasin-1 | 0.01 | 962 | potential |
| PRT062607 | 0.01 | 963 | potential |
| zatebradine | 0.01 | 964 | potential |
| desonide | 0.01 | 965 | potential |
| alcaftadine | 0.01 | 966 | potential |
| monobenzone | 0.01 | 967 | potential |
| rimexolone | 0.01 | 968 | potential |
| famprofazone | 0.01 | 969 | potential |
| olanzapine | 0.01 | 970 | potential |
| dextrose | 0.01 | 971 | potential |
| piretanide | 0.01 | 972 | potential |
| tofogliflozin | 0.01 | 973 | potential |
| BMS-649 | 0.01 | 974 | potential |
| benzoic-acid | 0.01 | 975 | potential |
| theobromine | 0.01 | 976 | potential |
| FG-2216 | 0.01 | 977 | potential |
| phenformin | 0.01 | 978 | potential |
| GSK461364 | 0.01 | 979 | potential |
| L-693403 | 0.01 | 980 | potential |
| artemisinin | 0.01 | 981 | potential |
| KU-55933 | 0.01 | 982 | potential |
| omidazole | 0.01 | 983 | potential |
| tideglusib | 0.01 | 984 | potential |
| bevirimat-dimeglumine | 0.01 | 985 | potential |
| dianhydrogalactitol | 0.01 | 986 | potential |
| imidafenacin | 0.01 | 987 | potential |
| decitabine | 0.01 | 988 | potential |
| 6,_7-dehydro-17- | 0.01 | 989 | potential |
| acetoxy-progesterone | | | |
| dehydroacetic-acid | 0.01 | 990 | potential |
| dizocilpine-(+) | 0.01 | 991 | potential |
| nalfurafine | 0.01 | 992 | potential |
| GDC-0879 | 0.01 | 993 | potential |
| epicatechin-gallate-(−) | 0.01 | 994 | potential |
| oleuropein | 0.01 | 995 | potential |
| eprobemide | 0.01 | 996 | potential |
| roscovitine | 0.01 | 997 | potential |
| fludrocortisone-acetate | 0.01 | 998 | potential |
| pacritinib | 0.01 | 999 | potential |
| R-59022 | 0.01 | 1000 | potential |
| E-2012 | 0.01 | 1001 | potential |
| primidone | 0.01 | 1002 | potential |
| rimcazole | 0.01 | 1003 | potential |
| metacetamol | 0.01 | 1004 | potential |
| RWJ-21757 | 0.01 | 1005 | potential |
| deflazacort | 0.01 | 1006 | potential |
| AM-630 | 0.01 | 1007 | potential |
| gallamine-triethiodide | 0.01 | 1008 | potential |
| tubastatin-a | 0.01 | 1009 | potential |
| CC-401 | 0.01 | 1010 | potential |
| apricitabine | 0.01 | 1011 | potential |
| AZD7687 | 0.01 | 1012 | potential |
| plurisin-#1 | 0.01 | 1013 | potential |
| lorlatinib | 0.01 | 1014 | potential |
| CT-7758 | 0.01 | 1015 | potential |
| gadodiamide | 0.01 | 1016 | potential |
| amcinonide | 0.01 | 1017 | potential |
| isoliquiritigenin | 0.01 | 1018 | potential |
| dabrafenib | 0.01 | 1019 | potential |
| sitafloxacin | 0.01 | 1020 | potential |
| gidazepam | 0.01 | 1021 | potential |
| PQ-401 | 0.01 | 1022 | potential |
| 4-PPBP | 0.01 | 1023 | potential |
| A-769662 | 0.01 | 1024 | potential |
| ADL5859 | 0.01 | 1025 | potential |
| mecamylamine | 0.01 | 1026 | potential |
| perhexiline | 0.01 | 1027 | potential |
| 1-EBIO | 0.01 | 1028 | potential |
| daltroban | 0.01 | 1029 | potential |
| haloperidol-decanoate | 0.01 | 1030 | potential |
| isoquinoline,-6,7-dimethoxy-1- | 0.01 | 1031 | potential |
| methyl-1,2,3,4-tetrahydro, | | | |
| niceritrol | 0.01 | 1032 | potential |
| fenoprofen | 0.01 | 1033 | potential |
| ivermectin | 0.01 | 1034 | potential |
| fenobam | 0.01 | 1035 | potential |
| veliflapon | 0.01 | 1036 | potential |
| GS-9620 | 0.01 | 1037 | potential |
| torin-2 | 0.01 | 1038 | potential |
| nevirapine | 0.01 | 1039 | potential |
| hydrocortisone-acetate | 0.01 | 1040 | potential |
| glycerol-monolaurate | 0.01 | 1041 | potential |
| diaveridine | 0.01 | 1042 | potential |
| val | 0.01 | 1043 | potential |
| cyclovirobuxin-d | 0.01 | 1044 | potential |
| CEP-32496 | 0.01 | 1045 | potential |
| chloralose | 0.01 | 1046 | potential |
| gamma-aminobutyric-acid | 0.01 | 1047 | potential |
| 7-aminocephalosporanic-acid | 0.01 | 1048 | potential |
| amibegron | 0.01 | 1049 | potential |
| CH5132799 | 0.01 | 1050 | potential |
| leflunomide | 0.01 | 1051 | potential |
| BML-190 | 0.01 | 1052 | potential |
| doxapram | 0.01 | 1053 | potential |
| amoxapine | 0.01 | 1054 | potential |
| FERb-033 | 0.01 | 1055 | potential |
| lubiprostone | 0.01 | 1056 | potential |
| 9-aminoacridine | 0.01 | 1057 | potential |
| CPI-1189 | 0.01 | 1058 | potential |
| pipenzolate | 0.01 | 1059 | potential |
| phensuximide | 0.01 | 1060 | potential |
| fadrozole | 0.01 | 1061 | potential |
| atomoxetine | 0.01 | 1062 | potential |
| BU226 | 0.01 | 1063 | potential |
| 3-CPMT | 0.01 | 1064 | potential |
| RS-56812 | 0.01 | 1065 | potential |
| org-27569 | 0.01 | 1066 | potential |
| tiratricol | 0.01 | 1067 | potential |
| isopropyl-palmitate | 0.01 | 1068 | potential |
| D-Serine | 0.01 | 1069 | potential |
| bithionol | 0.01 | 1070 | potential |
| ezetimibe | 0.01 | 1071 | potential |
| EO-1428 | 0.01 | 1072 | potential |
| desloratadine | 0.01 | 1073 | potential |
| maropitant | 0.01 | 1074 | potential |
| silodosin | 0.01 | 1075 | potential |
| isradipine | 0.01 | 1076 | potential |
| miltefosine | 0.01 | 1077 | potential |
| U-50488-(−) | 0.01 | 1078 | potential |
| ezetimibe | 0.01 | 1079 | potential |
| alpha-methylhistamine- | 0.01 | 1080 | potential |
| dihydrobromide-(S)-(+) | | | |
| narlaprevir | 0.01 | 1081 | potential |
| ibuprofen-piconol | 0.01 | 1082 | potential |
| ambroxol | 0.01 | 1083 | potential |
| dienestrol | 0.01 | 1084 | potential |
| NSC-4644 | 0.01 | 1085 | potential |
| bosutinib | 0.01 | 1086 | potential |
| ercalcitriol | 0.01 | 1087 | potential |
| cycloheximide | 0.01 | 1088 | potential |
| monoctanoin | 0.01 | 1089 | potential |
| testosterone | 0.01 | 1090 | potential |
| MK-5108 | 0.01 | 1091 | potential |
| MTPG | 0.01 | 1092 | potential |
| methoxsalen | 0.01 | 1093 | potential |
| purmorphamine | 0.01 | 1094 | potential |
| duloxetine | 0.01 | 1095 | potential |
| hydroxytacrine-maleate-(r,s) | 0.01 | 1096 | potential |
| pumosetrag | 0.01 | 1097 | potential |
| dichlorisone-acetate | 0.01 | 1098 | potential |
| orbifloxacin | 0.01 | 1099 | potential |
| methacycline | 0.01 | 1100 | potential |
| trans-4-Hydroxycrotonic-acid | 0.01 | 1101 | potential |
| testosterone-undecanoate | 0.01 | 1102 | potential |
| cotinine | 0.01 | 1103 | potential |
| AMG-319 | 0.01 | 1104 | potential |
| bismuth(iii)- | 0.01 | 1105 | potential |
| trifluoromethanesulfonate | | | |
| epinastine | 0.01 | 1106 | potential |
| imetit | 0.01 | 1107 | potential |
| lonafamib | 0.01 | 1108 | potential |
| suramin | 0.01 | 1109 | potential |
| mepyramine | 0.01 | 1110 | potential |
| hexamethonium | 0.01 | 1111 | potential |
| enflurane | 0.01 | 1112 | potential |
| rasagiline | 0.01 | 1113 | potential |
| BD-1063 | 0.01 | 1114 | potential |
| anpirtoline | 0.01 | 1115 | potential |
| R-1479 | 0.01 | 1116 | potential |
| 20-hydroxyecdysone | 0.01 | 1117 | potential |
| decoquinate | 0.01 | 1118 | potential |
| glimepiride | 0.01 | 1119 | potential |
| nitarsone | 0.01 | 1120 | potential |
| ritodrine | 0.01 | 1121 | potential |
| NVP-BEZ235 | 0.01 | 1122 | potential |
| metitepine | 0.01 | 1123 | potential |
| doxercalciferol | 0.01 | 1124 | potential |
| dronedarone | 0.01 | 1125 | potential |
| metaproterenol | 0.01 | 1126 | potential |
| tilmicosin | 0.01 | 1127 | potential |
| BAX-channel-blocker | 0.01 | 1128 | potential |
| simeprevir | 0.01 | 1129 | potential |
| etravirine | 0.01 | 1130 | potential |
| JTE-607 | 0.01 | 1131 | potential |
| prochlorperazine | 0.01 | 1132 | potential |
| abiraterone-acetate | 0.01 | 1133 | potential |
| foropafant | 0.01 | 1134 | potential |
| estramustine | 0.01 | 1135 | potential |
| tulobuterol | 0.01 | 1136 | potential |
| PF-05190457 | 0.01 | 1137 | potential |
| demecarium | 0.01 | 1138 | potential |
| KI-8751 | 0.01 | 1139 | potential |
| NE-100 | 0.01 | 1140 | potential |
| asiatic-acid | 0.01 | 1141 | potential |
| moxidectin | 0.01 | 1142 | potential |
| tonabersat | 0.01 | 1143 | potential |
| AMG319 | 0.01 | 1144 | potential |
| ethenzamide | 0.01 | 1145 | potential |
| hycanthone | 0.01 | 1146 | potential |
| pasireotide | 0.01 | 1147 | potential |
| sufentanil | 0.01 | 1148 | potential |
| selamectin | 0.01 | 1149 | potential |
| PHA-848125 | 0.01 | 1150 | potential |
| CGP-71683 | 0.01 | 1151 | potential |
| BAY-K-8644-(s)-(−) | 0.01 | 1152 | potential |
| mometasone-furoate | 0.01 | 1153 | potential |
| PX-I2 | 0.01 | 1154 | potential |
| vitamin-b12 | 0.01 | 1155 | potential |
| EPZ-5676 | 0.01 | 1156 | potential |
| cisplatin | 0.01 | 1157 | potential |
| halcinonide | 0.01 | 1158 | potential |
| bupranolol | 0.01 | 1159 | potential |
| NVP-DPP728 | 0.01 | 1160 | potential |
| minocycline | 0.01 | 1161 | potential |
| nor-Binaltorphimine-dihydrochloride | 0.01 | 1162 | potential |
| SB-202190 | 0.01 | 1163 | potential |
| biperiden | 0.01 | 1164 | potential |
| GS-9973 | 0.01 | 1165 | potential |
| limaprost-alfadex | 0.01 | 1166 | potential |
| GKT137831 | 0.01 | 1167 | potential |
| ketanserin | 0.01 | 1168 | potential |
| thioproperazine | 0.01 | 1169 | potential |
| CB-03-01 | 0.01 | 1170 | potential |
| aceclofenac | 0.01 | 1171 | potential |
| RS-39604 | 0.01 | 1172 | potential |
| sulfaphenazole | 0.01 | 1173 | potential |
| clonazepam | 0.01 | 1174 | potential |
| BTS-54505 | 0.01 | 1175 | potential |
| dioscin | 0.01 | 1176 | potential |
| sodium-glucoheptonate | 0.01 | 1177 | potential |
| papaverine | 0.01 | 1178 | potential |
| toremifene | 0.01 | 1179 | potential |
| dizocilpine-(−) | 0.01 | 1180 | potential |
| pidolic-acid | 0.01 | 1181 | potential |
| mequinol | 0.01 | 1182 | potential |
| preclamol | 0.01 | 1183 | potential |
| zanamivir | 0.01 | 1184 | potential |
| tizanidine | 0.01 | 1185 | potential |
| PIK-93 | 0.01 | 1186 | potential |
| hydroflumethiazide | 0.01 | 1187 | potential |
| anisodamine | 0.01 | 1188 | potential |
| WP1130 | 0.01 | 1189 | potential |
| sulpiride | 0.01 | 1190 | potential |
| BW-180C | 0.01 | 1191 | potential |
| butoconazole | 0.01 | 1192 | potential |
| fimasartan | 0.01 | 1193 | potential |
| bromopride | 0.01 | 1194 | potential |
| 9-aminoacridine | 0.01 | 1195 | potential |
| penciclovir | 0.01 | 1196 | potential |
| vigabatrin | 0.01 | 1197 | potential |
| amprolium | 0.01 | 1198 | potential |
| pranlukast | 0.01 | 1199 | potential |
| sulfaguanidine | 0.01 | 1200 | potential |
| N-benzylnaltrindole | 0.01 | 1201 | potential |
| trihexyphenidyl | 0.01 | 1202 | potential |
| RS-102895 | 0.01 | 1203 | potential |
| MK-2461 | 0.01 | 1204 | potential |
| TCS-359 | 0.01 | 1205 | potential |
| delanzomib | 0.01 | 1206 | potential |
| elafibranor | 0.01 | 1207 | potential |
| procaine | 0.01 | 1208 | potential |
| clidinium | 0.01 | 1209 | potential |
| adiporon | 0.01 | 1210 | potential |
| ractopamine | 0.01 | 1211 | potential |
| BTZ043-racemate | 0.01 | 1212 | potential |
| pyridoxine | 0.01 | 1213 | potential |
| fdcyd | 0.01 | 1214 | potential |
| phenolsulfonphthalein | 0.01 | 1215 | potential |
| chlorcyclizine | 0.01 | 1216 | potential |
| josamycin | 0.01 | 1217 | potential |
| homochlorcyclizine | 0.01 | 1218 | potential |
| clemizole | 0.01 | 1219 | potential |
| AMPA-(RS) | 0.01 | 1220 | potential |
| mequinol | 0.01 | 1221 | potential |
| cortodoxone | 0.01 | 1222 | potential |
| oltipraz | 0.01 | 1223 | potential |
| ICI-185,282 | 0.01 | 1224 | potential |
| xilobam | 0.01 | 1225 | potential |
| fluphenazine | 0.01 | 1226 | potential |
| methicillin | 0.01 | 1227 | potential |
| LY2090314 | 0.01 | 1228 | potential |
| cinalukast | 0.01 | 1229 | potential |
| chloroquine | 0.01 | 1230 | potential |
| copper-histidine | 0.01 | 1231 | potential |
| crizotinib-(S) | 0.01 | 1232 | potential |
| meprobamate | 0.01 | 1233 | potential |
| TGR-1202 | 0.01 | 1234 | potential |
| AZD1283 | 0.01 | 1235 | potential |
| CID-5458317 | 0.01 | 1236 | potential |
| metixene | 0.01 | 1237 | potential |
| SID-7969543 | 0.01 | 1238 | potential |
| presatovir | 0.01 | 1239 | potential |
| 4-mu-8C | 0.01 | 1240 | potential |
| drofenine | 0.01 | 1241 | potential |
| VGX-1027 | 0.01 | 1242 | potential |
| NMS-E973 | 0.01 | 1243 | potential |
| nanchangmycin | 0.01 | 1244 | potential |
| abafungin | 0.01 | 1245 | potential |
| prednisolone-tebutate | 0.01 | 1246 | potential |
| 1,3-dipropyl-8-phenylxanthine | 0.01 | 1247 | potential |
| SK&F-10047-(+) | 0.01 | 1248 | potential |
| defactinib | 0.01 | 1249 | potential |
| crotamiton | 0.01 | 1250 | potential |
| ganciclovir | 0.01 | 1251 | potential |
| INCB-024360 | 0.01 | 1252 | potential |
| navarixin | 0.01 | 1253 | potential |
| otenzepad | 0.01 | 1254 | potential |
| TG-003 | 0.01 | 1255 | potential |
| saxagliptin | 0.01 | 1256 | potential |
| clindamycin-palmitate | 0.01 | 1257 | potential |
| terguride | 0.01 | 1258 | potential |
| desipramine | 0.01 | 1259 | potential |
| colforsin-daproate | 0.01 | 1260 | potential |
| 5-carboxamidotryptamine | 0.01 | 1261 | potential |
| dextran | 0.01 | 1262 | potential |
| dixanthogen | 0.01 | 1263 | potential |
| sodium-oxybate | 0.01 | 1264 | potential |
| lidoflazine | 0.01 | 1265 | potential |
| enciprazine | 0.01 | 1266 | potential |
| AM-404 | 0.01 | 1267 | potential |
| sodium-stibogluconate | 0.01 | 1268 | potential |
| UNC0646 | 0.01 | 1269 | potential |
| BMS-707035 | 0.01 | 1270 | potential |
| butalbital | 0.01 | 1271 | potential |
| metenkephalin | 0.01 | 1272 | potential |
| alaproclate | 0.01 | 1273 | potential |
| aminocaproic-acid | 0.01 | 1274 | potential |
| nicotinamide | 0.01 | 1275 | potential |
| CS-110266 | 0.01 | 1276 | potential |
| RS-127445 | 0.01 | 1277 | potential |
| betamethasone-acetate | 0.01 | 1278 | potential |
| flunarizine | 0.01 | 1279 | potential |
| selinexor | 0.01 | 1280 | potential |
| chloroxoquinoline | 0.01 | 1281 | potential |
| vesnarinone | 0.01 | 1282 | potential |
| leucovorin | 0.01 | 1283 | potential |
| dazoxiben | 0.01 | 1284 | potential |
| vatalanib | 0.01 | 1285 | potential |
| VU0155069 | 0.01 | 1286 | potential |
| lidocaine | 0.01 | 1287 | potential |
| ropinirole | 0.01 | 1288 | potential |
| pyridoxal | 0.01 | 1289 | potential |
| arofylline | 0.01 | 1290 | potential |
| estradiol-benzoate | 0.01 | 1291 | potential |
| spaglumic-acid | 0.01 | 1292 | potential |
| gadobutrol | 0.01 | 1293 | potential |
| reparixin | 0.01 | 1294 | potential |
| N6022 | 0.01 | 1295 | potential |
| bemesetron | 0.01 | 1296 | potential |
| aloxistatin | 0.01 | 1297 | potential |
| aloperine | 0.01 | 1298 | potential |
| carsalam | 0.01 | 1299 | potential |
| butylated-hydroxytoluene | 0.01 | 1300 | potential |
| 10-hydroxycamptothecin | 0.01 | 1301 | potential |
| cnipagliflozin | 0.01 | 1302 | potential |
| thiocolchicoside | 0.01 | 1303 | potential |
| gabazine | 0.01 | 1304 | potential |
| methscopolamine | 0.01 | 1305 | potential |
| sipatrigine | 0.01 | 1306 | potential |
| tangeritin | 0.01 | 1307 | potential |
| GP2a | 0.01 | 1308 | potential |
| L-Aspartic-Acid | 0.01 | 1309 | potential |
| butofilolol | 0.01 | 1310 | potential |
| alibendol | 0.01 | 1311 | potential |
| SAR-245409 | 0.01 | 1312 | potential |
| tromaril | 0.01 | 1313 | potential |
| molindone | 0.01 | 1314 | potential |
| cyclosporin-a | 0.01 | 1315 | potential |
| amperozide | 0.01 | 1316 | potential |
| etilevodopa | 0.01 | 1317 | potential |
| tiaprofenic-acid | 0.01 | 1318 | potential |
| taurocholate | 0.01 | 1319 | potential |
| L-ergothioneine | 0.01 | 1320 | potential |
| taranabant | 0.01 | 1321 | potential |
| iopanic-acid | 0.01 | 1322 | potential |
| PSB-36 | 0.01 | 1323 | potential |
| fluorometholone-acetate | 0.01 | 1324 | potential |
| parthenolide-(altemate-stereo) | 0.01 | 1325 | potential |
| GDC-0941 | 0.01 | 1326 | potential |
| NSC-9965 | 0.01 | 1327 | potential |
| GR-159897 | 0.01 | 1328 | potential |
| pindolol | 0.01 | 1329 | potential |
| eugenol | 0.01 | 1330 | potential |
| pardoprunox | 0.01 | 1331 | potential |
| 2-hydroxyflutamide | 0.01 | 1332 | potential |
| SNX-5422 | 0.01 | 1333 | potential |
| clonixin-lysinate | 0.01 | 1334 | potential |
| malic-acid | 0.01 | 1335 | potential |
| cinepazet | 0.01 | 1336 | potential |
| S-methylcysteine | 0.01 | 1337 | potential |
| LX1031 | 0.01 | 1338 | potential |
| meloxicam | 0.01 | 1339 | potential |
| digitoxin | 0.01 | 1340 | potential |
| ellagic-acid | 0.01 | 1341 | potential |
| azeliragon | 0.01 | 1342 | potential |
| wnt-c59 | 0.01 | 1343 | potential |
| oxacillin | 0.01 | 1344 | potential |
| fluroxene | 0.01 | 1345 | potential |
| naloxone-benzoylhydrazone | 0.01 | 1346 | potential |
| sulisobenzone | 0.01 | 1347 | potential |
| 5-HMF | 0.01 | 1348 | potential |
| licochalcone-a | 0.01 | 1349 | potential |
| AG-1024 | 0.01 | 1350 | potential |
| tenofovir-disoproxil | 0.01 | 1351 | potential |
| butein | 0.01 | 1352 | potential |
| MLN2480 | 0.01 | 1353 | potential |
| bilastine | 0.01 | 1354 | potential |
| ifenprodil | 0.01 | 1355 | potential |
| ZM-323881 | 0.01 | 1356 | potential |
| olmesartan | 0.01 | 1357 | potential |
| bindarit | 0.01 | 1358 | potential |
| ameltolide | 0.01 | 1359 | potential |
| KU14R | 0.01 | 1360 | potential |
| enoxolone | 0.01 | 1361 | potential |
| medetomidine | 0.01 | 1362 | potential |
| asiatic-acid | 0.01 | 1363 | potential |
| succinylsulfathiazole | 0.01 | 1364 | potential |
| flutamide | 0.01 | 1365 | potential |
| entecavir | 0.01 | 1366 | potential |
| acamprosate | 0.01 | 1367 | potential |
| phthalylsulfathiazole | 0.01 | 1368 | potential |
| isoflupredone-acetate | 0.01 | 1369 | potential |
| paeonol | 0.01 | 1370 | potential |
| amidopyrine | 0.01 | 1371 | potential |
| BAY-85-8050 | 0.01 | 1372 | potential |
| 4-HQN | 0.01 | 1373 | potential |
| fmoc-1-leucine | 0.01 | 1374 | potential |
| piperacillin | 0.01 | 1375 | potential |
| digoxigenin | 0.01 | 1376 | potential |
| oxatomide | 0.01 | 1377 | potential |
| phenprobamate | 0.01 | 1378 | potential |
| paraxanthine | 0.01 | 1379 | potential |
| dantron | 0.01 | 1380 | potential |
| ZM-241385 | 0.01 | 1381 | potential |
| OSI-027 | 0.01 | 1382 | potential |
| genipin | 0.01 | 1383 | potential |
| SB-271046 | 0.01 | 1384 | potential |
| PRE-084 | 0.01 | 1385 | potential |
| gallopamil | 0.01 | 1386 | potential |
| rosoxacin | 0.01 | 1387 | potential |
| levallorphan | 0.01 | 1388 | potential |
| amiodarone | 0.01 | 1389 | potential |
| imipramine | 0.01 | 1390 | potential |
| flibanserin | 0.01 | 1391 | potential |
| bropirimine | 0.01 | 1392 | potential |
| perospirone | 0.01 | 1393 | potential |
| carbazochrome | 0.01 | 1394 | potential |
| PG-9 | 0.01 | 1395 | potential |
| melengestrol-acetate | 0.01 | 1396 | potential |
| diazoxide | 0.01 | 1397 | potential |
| cyproheptadine | 0.01 | 1398 | potential |
| nicotine | 0.01 | 1399 | potential |
| proxymetacaine | 0.01 | 1400 | potential |
| GSK2126458 | 0.01 | 1401 | potential |
| YM022 | 0.01 | 1402 | potential |
| ibutilide | 0.01 | 1403 | potential |
| SCH-23390 | 0.01 | 1404 | potential |
| amikacin | 0.01 | 1405 | potential |
| ardeparin | 0.01 | 1406 | potential |
| estradiol-valerate | 0.01 | 1407 | potential |
| DFB | 0.01 | 1408 | potential |
| pentolinium | 0.01 | 1409 | potential |
| azilsartan | 0.01 | 1410 | potential |
| piperidolate | 0.01 | 1411 | potential |
| novobiocin | 0.01 | 1412 | potential |
| eliglustat | 0.01 | 1413 | potential |
| tolimidone | 0.01 | 1414 | potential |
| vancomycin | 0.01 | 1415 | potential |
| bephenium-hydroxynaphthoate | 0.01 | 1416 | potential |
| IEM1460 | 0.01 | 1417 | potential |
| RU-58841 | 0.01 | 1418 | potential |
| lumiracoxib | 0.01 | 1419 | potential |
| epitiostanol | 0.01 | 1420 | potential |
| shikimic-acid | 0.01 | 1421 | potential |
| SCP-1 | 0.01 | 1422 | potential |
| dicycloverine | 0.01 | 1423 | potential |
| diflunisal | 0.01 | 1424 | potential |
| dichlorophen | 0.01 | 1425 | potential |
| trigonelline | 0.01 | 1426 | potential |
| felbamate | 0.01 | 1427 | potential |
| amineptine | 0.01 | 1428 | potential |
| geniposide | 0.01 | 1429 | potential |
| racecadotril | 0.01 | 1430 | potential |
| flucytosine | 0.01 | 1431 | potential |
| uracil | 0.01 | 1432 | potential |
| proxyfan | 0.01 | 1433 | potential |
| spermine | 0.01 | 1434 | potential |
| amlexanox | 0.01 | 1435 | potential |
| bentiromide | 0.01 | 1436 | potential |
| GSK429286A | 0.01 | 1437 | potential |
| CX-516 | 0.01 | 1438 | potential |
| zaltidine | 0.01 | 1439 | potential |
| N-acetylglycyl-D-glutamic-acid | 0.01 | 1440 | potential |
| emtricitabine | 0.01 | 1441 | potential |
| CPP | 0.01 | 1442 | potential |
| betamethasone-valerate | 0.01 | 1443 | potential |
| plovamer-acetate | 0.01 | 1444 | potential |
| benidipine | 0.01 | 1445 | potential |
| triclabendazole | 0.01 | 1446 | potential |
| TCS2002 | 0.01 | 1447 | potential |
| suritozole | 0.01 | 1448 | potential |
| SB-218078 | 0.01 | 1449 | potential |
| lonidamine | 0.01 | 1450 | potential |
| thiethylperazine | 0.01 | 1451 | potential |
| hyperin | 0.01 | 1452 | potential |
| torcetrapib | 0.01 | 1453 | potential |
| beclomethasone | 0.01 | 1454 | potential |
| andarine | 0.01 | 1455 | potential |
| falecalcitriol | 0.01 | 1456 | potential |
| pralidoxime | 0.01 | 1457 | potential |
| medroxyprogesterone | 0.01 | 1458 | potential |
| amflutizole | 0.01 | 1459 | potential |
| aprepitant | 0.01 | 1460 | potential |
| vercirnon | 0.01 | 1461 | potential |
| ondansetron | 0.01 | 1462 | potential |
| TMC-353121 | 0.01 | 1463 | potential |
| guanadrel | 0.01 | 1464 | potential |
| formononetin | 0.01 | 1465 | potential |
| oxybenzone | 0.01 | 1466 | potential |
| repsox | 0.01 | 1467 | potential |
| caspofungin-acetate | 0.01 | 1468 | potential |
| AZ-628 | 0.01 | 1469 | potential |
| AP1903 | 0.01 | 1470 | potential |
| GW-4064 | 0.01 | 1471 | potential |
| trepibutone | 0.01 | 1472 | potential |
| apoptosis-activator-II | 0.01 | 1473 | potential |
| tyrphostin-AG-494 | 0.01 | 1474 | potential |
| metharbital | 0.01 | 1475 | potential |
| spiradoline | 0.01 | 1476 | potential |
| laropiprant | 0.01 | 1477 | potential |
| NSC-405020 | 0.01 | 1478 | potential |
| lansoprazole | 0.01 | 1479 | potential |
| BADGE | 0.01 | 1480 | potential |
| cleviprex | 0.01 | 1481 | potential |
| cilostamide | 0.01 | 1482 | potential |
| nialamide | 0.01 | 1483 | potential |
| TAK-715 | 0.01 | 1484 | potential |
| CGK-733 | 0.01 | 1485 | potential |
| fenretinide | 0.01 | 1486 | potential |
| istradefylline | 0.01 | 1487 | potential |
| benproperine | 0.01 | 1488 | potential |
| blonanserin | 0.01 | 1489 | potential |
| almorexant | 0.01 | 1490 | potential |
| 06-Benzylguanine | 0.01 | 1491 | potential |
| ozolinone | 0.01 | 1492 | potential |
| SB-366791 | 0.01 | 1493 | potential |
| selexipag | 0.01 | 1494 | potential |
| acenocoumarol | 0.01 | 1495 | potential |
| blebbistatin-(−) | 0.01 | 1496 | potential |
| fosfosal | 0.01 | 1497 | potential |
| ranirestat | 0.01 | 1498 | potential |
| carbimazole | 0.01 | 1499 | potential |
| tenofovir | 0.01 | 1500 | potential |
| AZD9668 | 0.01 | 1501 | potential |
| tolamolol | 0.01 | 1502 | potential |
| triflupromazine | 0.01 | 1503 | potential |
| N-acetyl-tyrosine | 0.01 | 1504 | potential |
| sodium-gluconate | 0.01 | 1505 | potential |
| L-168049 | 0.01 | 1506 | potential |
| sitaxentan | 0.01 | 1507 | potential |
| meparfylon | 0.01 | 1508 | potential |
| omarigliptin | 0.01 | 1509 | potential |
| tyrphostin-AG-1296 | 0.01 | 1510 | potential |
| galantamine | 0.01 | 1511 | potential |
| ICI-192605 | 0.01 | 1512 | potential |
| AGI-5198 | 0.01 | 1513 | potential |
| mafenide | 0.01 | 1514 | potential |
| siramesine | 0.01 | 1515 | potential |
| diphenhydramine | 0.01 | 1516 | potential |
| nibentan | 0.01 | 1517 | potential |
| mercaptosuccinic-acid | 0.01 | 1518 | potential |
| prednisolone-acetate | 0.01 | 1519 | potential |
| SAR407899 | 0.01 | 1520 | potential |
| otilonium | 0.01 | 1521 | potential |
| VT-464 | 0.01 | 1522 | potential |
| CPCCOEt | 0.01 | 1523 | potential |
| BU-224 | 0.01 | 1524 | potential |
| acefylline | 0.01 | 1525 | potential |
| SDZ-NKT-343 | 0.01 | 1526 | potential |
| GSK-0660 | 0.01 | 1527 | potential |
| sobetirome | 0.01 | 1528 | potential |
| gly-gln | 0.01 | 1529 | potential |
| acadesine | 0.00 | 1530 | potential |
| olomoucine | 0.00 | 1531 | potential |
| FK-888 | 0.00 | 1532 | potential |
| dabigatran | 0.00 | 1533 | potential |
| cyclopenthiazide | 0.00 | 1534 | potential |
| tolazamide | 0.00 | 1535 | potential |
| propylthiouracil | 0.00 | 1536 | potential |
| proxyphylline | 0.00 | 1537 | potential |
| toltrazuril | 0.00 | 1538 | potential |
| betulinic-acid | 0.00 | 1539 | potential |
| indinavir | 0.00 | 1540 | potential |
| norcyclobenzaprine | 0.00 | 1541 | potential |
| voglibose | 0.00 | 1542 | potential |
| nafadotride | 0.00 | 1543 | potential |
| trap-101 | 0.00 | 1544 | potential |
| pioglitazone | 0.00 | 1545 | potential |
| flupentixol | 0.00 | 1546 | potential |
| metamizole | 0.00 | 1547 | potential |
| Ro-9187 | 0.00 | 1548 | potential |
| sparteine-sulfate | 0.00 | 1549 | potential |
| WHI-P154 | 0.00 | 1550 | potential |
| proadifen | 0.00 | 1551 | potential |
| prednisolone | 0.00 | 1552 | potential |
| phenothiazine | 0.00 | 1553 | potential |
| alacepril | 0.00 | 1554 | potential |
| desoxycortone | 0.00 | 1555 | potential |
| clemastine | 0.00 | 1556 | potential |
| oxandrolone | 0.00 | 1557 | potential |
| flumethasone | 0.00 | 1558 | potential |
| YM-511 | 0.00 | 1559 | potential |
| testolactone | 0.00 | 1560 | potential |
| BIBR-1532 | 0.00 | 1561 | potential |
| lurasidone | 0.00 | 1562 | potential |
| F351 | 0.00 | 1563 | potential |
| carbadox | 0.00 | 1564 | potential |
| suprofen | 0.00 | 1565 | potential |
| ginkgolide-b | 0.00 | 1566 | potential |
| TAK-875 | 0.00 | 1567 | potential |
| sonidegib | 0.00 | 1568 | potential |
| tepoxalin | 0.00 | 1569 | potential |
| lincomycin | 0.00 | 1570 | potential |
| vanoxerine | 0.00 | 1571 | potential |
| iodixanol | 0.00 | 1572 | potential |
| uridine-triacetate | 0.00 | 1573 | potential |
| clobutinol | 0.00 | 1574 | potential |
| altinicline | 0.00 | 1575 | potential |
| L-161982 | 0.00 | 1576 | potential |
| BMS-299897 | 0.00 | 1577 | potential |
| imatinib | 0.00 | 1578 | potential |
| BTS | 0.00 | 1579 | potential |
| CEP-33779 | 0.00 | 1580 | potential |
| mabuterol | 0.00 | 1581 | potential |
| meisoindigo | 0.00 | 1582 | potential |
| dichloroacetate | 0.00 | 1583 | potential |
| LY2608204 | 0.00 | 1584 | potential |
| UH-232-(+) | 0.00 | 1585 | potential |
| SKA-31 | 0.00 | 1586 | potential |
| pregnenolone | 0.00 | 1587 | potential |
| DPO-1 | 0.00 | 1588 | potential |
| linsitinib | 0.00 | 1589 | potential |
| JNJ-7777120 | 0.00 | 1590 | potential |
| cefonicid | 0.00 | 1591 | potential |
| cinromide | 0.00 | 1592 | potential |
| estetrol | 0.00 | 1593 | potential |
| sapropterin | 0.00 | 1594 | potential |
| MK-8033 | 0.00 | 1595 | potential |
| isometheptene-mucate | 0.00 | 1596 | potential |
| cerulenin | 0.00 | 1597 | potential |
| picotamide | 0.00 | 1598 | potential |
| allopurinol-riboside | 0.00 | 1599 | potential |
| ribitol | 0.00 | 1600 | potential |
| exisulind | 0.00 | 1601 | potential |
| pefloxacin | 0.00 | 1602 | potential |
| almitrine | 0.00 | 1603 | potential |
| oxybutynin | 0.00 | 1604 | potential |
| orphenadrine | 0.00 | 1605 | potential |
| dapsone | 0.00 | 1606 | potential |
| ttiacetin | 0.00 | 1607 | potential |
| miocamycin | 0.00 | 1608 | potential |
| cefetamet-pivoxil | 0.00 | 1609 | potential |
| MK-0752 | 0.00 | 1610 | potential |
| nimorazole | 0.00 | 1611 | potential |
| LY223982 | 0.00 | 1612 | potential |
| alpha-tochopheryl-acetate | 0.00 | 1613 | potential |
| tandutinib | 0.00 | 1614 | potential |
| buphenine | 0.00 | 1615 | potential |
| perflubron | 0.00 | 1616 | potential |
| MK-3207 | 0.00 | 1617 | potential |
| salirasib | 0.00 | 1618 | potential |
| beclomethasone-dipropionate | 0.00 | 1619 | potential |
| SC-12267 | 0.00 | 1620 | potential |
| ME0328 | 0.00 | 1621 | potential |
| ketorolac | 0.00 | 1622 | potential |
| thiazovivin | 0.00 | 1623 | potential |
| pergolide | 0.00 | 1624 | potential |
| nebivolol | 0.00 | 1625 | potential |
| salicylamide | 0.00 | 1626 | potential |
| isoetharine | 0.00 | 1627 | potential |
| lorglumide | 0.00 | 1628 | potential |
| canrenone | 0.00 | 1629 | potential |
| urea | 0.00 | 1630 | potential |
| lesinurad | 0.00 | 1631 | potential |
| methoprene-(s) | 0.00 | 1632 | potential |
| nonivamide | 0.00 | 1633 | potential |
| PACOCF3 | 0.00 | 1634 | potential |
| memantine | 0.00 | 1635 | potential |
| REV-5901 | 0.00 | 1636 | potential |
| nitrocaramiphen | 0.00 | 1637 | potential |
| INT-747 | 0.00 | 1638 | potential |
| regorafenib | 0.00 | 1639 | potential |
| adenosine | 0.00 | 1640 | potential |
| nitazoxanide | 0.00 | 1641 | potential |
| N-acetyltryptamine | 0.00 | 1642 | potential |
| resatorvid | 0.00 | 1643 | potential |
| tolmetin | 0.00 | 1644 | potential |
| phloretin | 0.00 | 1645 | potential |
| 5-hydroxytryptophan | 0.00 | 1646 | potential |
| capadenoson | 0.00 | 1647 | potential |
| PD-184352 | 0.00 | 1648 | potential |
| esmolol | 0.00 | 1649 | potential |
| bromantan | 0.00 | 1650 | potential |
| methyclothiazide | 0.00 | 1651 | potential |
| tetrabenazine | 0.00 | 1652 | potential |
| delavirdine | 0.00 | 1653 | potential |
| vecuronium | 0.00 | 1654 | potential |
| rucinol | 0.00 | 1655 | potential |
| celecoxib | 0.00 | 1656 | potential |
| tibolone | 0.00 | 1657 | potential |
| MEK1-2-inhibitor | 0.00 | 1658 | potential |
| odanacatib | 0.00 | 1659 | potential |
| 2-[1-(4-piperonyl)piperazinyl]- | 0.00 | 1660 | potential |
| benzothiazole | | | |
| BMS-927711 | 0.00 | 1661 | potential |
| rivanicline | 0.00 | 1662 | potential |
| sulfamethazine | 0.00 | 1663 | potential |
| propylhexedrine | 0.00 | 1664 | potential |
| irsogladine | 0.00 | 1665 | potential |
| AR-A014418 | 0.00 | 1666 | potential |
| difluprednate | 0.00 | 1667 | potential |
| clindamycin-phosphate | 0.00 | 1668 | potential |
| metaraminol | 0.00 | 1669 | potential |
| triamcinolone-acetonide | 0.00 | 1670 | potential |
| sotrastaurin | 0.00 | 1671 | potential |
| BMY-45778 | 0.00 | 1672 | potential |
| AM-92016 | 0.00 | 1673 | potential |
| alclofenac | 0.00 | 1674 | potential |
| PF-04457845 | 0.00 | 1675 | potential |
| JNJ-26990990 | 0.00 | 1676 | potential |
| BRL-37344 | 0.00 | 1677 | potential |
| medica-16 | 0.00 | 1678 | potential |
| BMS-599626 | 0.00 | 1679 | potential |
| thonzylamine | 0.00 | 1680 | potential |
| nithiamide | 0.00 | 1681 | potential |
| flunixin-meglumin | 0.00 | 1682 | potential |
| L-proline | 0.00 | 1683 | potential |
| tetracaine | 0.00 | 1684 | potential |
| anandamide | 0.00 | 1685 | potential |
| guanfacine | 0.00 | 1686 | potential |
| SKF-81297 | 0.00 | 1687 | potential |
| famciclovir | 0.00 | 1688 | potential |
| iopodic-acid | 0.00 | 1689 | potential |
| acetazolamide | 0.00 | 1690 | potential |
| voriconazole | 0.00 | 1691 | potential |
| pyrithione-zinc | 0.00 | 1692 | potential |
| flindokalner | 0.00 | 1693 | potential |
| BP-897 | 0.00 | 1694 | potential |
| parthenolide | 0.00 | 1695 | potential |
| idronoxil | 0.00 | 1696 | potential |
| ubenimex | 0.00 | 1697 | potential |
| trimipramine | 0.00 | 1698 | potential |
| dopamine | 0.00 | 1699 | potential |
| TAK-063 | 0.00 | 1700 | potential |
| lodoxamide | 0.00 | 1701 | potential |
| 4-P-PDOT | 0.00 | 1702 | potential |
| didox | 0.00 | 1703 | potential |
| 3-carboxy-4-hydroxyphenyl-glycine-(R) | 0.00 | 1704 | potential |
| dehydrocholate | 0.00 | 1705 | potential |
| LY215490 | 0.00 | 1706 | potential |
| DNQX | 0.00 | 1707 | potential |
| sulfamethazine | 0.00 | 1708 | potential |
| benzo[d]thiazole-2(3h)-thione | 0.00 | 1709 | potential |
| cis-urocanic acid | 0.00 | 1710 | potential |
| liranaftate | 0.00 | 1711 | potential |
| ceftibuten | 0.00 | 1712 | potential |
| dimemorfan | 0.00 | 1713 | potential |
| moxisylyte | 0.00 | 1714 | potential |
| chlorpropamide | 0.00 | 1715 | potential |
| beta-glycerophosphoric-acid | 0.00 | 1716 | potential |
| pamidronate | 0.00 | 1717 | potential |
| PIK-293 | 0.00 | 1718 | potential |
| fluorescein | 0.00 | 1719 | potential |
| leuprolide | 0.00 | 1720 | potential |
| aminothiazole | 0.00 | 1721 | potential |
| L-Hydroxyproline | 0.00 | 1722 | potential |
| avatrombopag | 0.00 | 1723 | potential |
| DY131 | 0.00 | 1724 | potential |
| roxithromycin | 0.00 | 1725 | potential |
| BMS-806 | 0.00 | 1726 | potential |
| enalaprilat | 0.00 | 1727 | potential |
| CH-170 | 0.00 | 1728 | potential |
| TMC647055 | 0.00 | 1729 | potential |
| JNJ-40411813 | 0.00 | 1730 | potential |
| PIK-294 | 0.00 | 1731 | potential |
| methscopolamine | 0.00 | 1732 | potential |
| AH11110 | 0.00 | 1733 | potential |
| 7-methylxanthine | 0.00 | 1734 | potential |
| orotic-acid | 0.00 | 1735 | potential |
| procyanidin-b-2 | 0.00 | 1736 | potential |
| sorbinil | 0.00 | 1737 | potential |
| pemirolast | 0.00 | 1738 | potential |
| bepridil | 0.00 | 1739 | potential |
| tiaramide | 0.00 | 1740 | potential |
| vilazodone | 0.00 | 1741 | potential |
| paritaprevir | 0.00 | 1742 | potential |
| mebeverine | 0.00 | 1743 | potential |
| CB-10-277 | 0.00 | 1744 | potential |
| sotalol | 0.00 | 1745 | potential |
| plerixafor | 0.00 | 1746 | potential |
| 2-Chloropyrazine | 0.00 | 1747 | potential |
| 7-keto-DHEA | 0.00 | 1748 | potential |
| vanillylacetone | 0.00 | 1749 | potential |
| telenzepine | 0.00 | 1750 | potential |
| olprinone | 0.00 | 1751 | potential |
| nomegestrol-acetate | 0.00 | 1752 | potential |
| estradiol | 0.00 | 1753 | potential |
| guanethidine | 0.00 | 1754 | potential |
| lornoxicam | 0.00 | 1755 | potential |
| satraplatin | 0.00 | 1756 | potential |
| tilorone | 0.00 | 1757 | potential |
| hydrocortisone-hemisuccinate | 0.00 | 1758 | potential |
| eptifibatide | 0.00 | 1759 | potential |
| pregabalin | 0.00 | 1760 | potential |
| solamargine | 0.00 | 1761 | potential |
| tebipenem | 0.00 | 1762 | potential |
| AGI-6780 | 0.00 | 1763 | potential |
| ethopabate | 0.00 | 1764 | potential |
| quinethazone | 0.00 | 1765 | potential |
| glutathione-monoisopropyl-ester | 0.00 | 1766 | potential |
| tyrphostin-AG-99 | 0.00 | 1767 | potential |
| medronic-acid | 0.00 | 1768 | potential |
| CP-945,598 | 0.00 | 1769 | potential |
| terlipressin | 0.00 | 1770 | potential |
| bivalirudin | 0.00 | 1771 | potential |
| benzotript | 0.00 | 1772 | potential |
| stattic | 0.00 | 1773 | potential |
| brivanib | 0.00 | 1774 | potential |
| LTA | 0.00 | 1775 | potential |
| gadoterate-meglumine | 0.00 | 1776 | potential |
| STF-62247 | 0.00 | 1777 | potential |
| hydroxyzine | 0.00 | 1778 | potential |
| trichloroethylene | 0.00 | 1779 | potential |
| SKF-86002 | 0.00 | 1780 | potential |
| sutezolid | 0.00 | 1781 | potential |
| phentermine | 0.00 | 1782 | potential |
| isoniazid | 0.00 | 1783 | potential |
| denotivir | 0.00 | 1784 | potential |
| SB-683698 | 0.00 | 1785 | potential |
| rotundine | 0.00 | 1786 | potential |
| acitazanolast | 0.00 | 1787 | potential |
| creatinine | 0.00 | 1788 | potential |
| donepezil | 0.00 | 1789 | potential |
| caprylic-acid | 0.00 | 1790 | potential |
| cilnidipine | 0.00 | 1791 | potential |
| eslicarbazepine-acetate | 0.00 | 1792 | potential |
| chrysophanic-acid | 0.00 | 1793 | potential |
| tobramycin | 0.00 | 1794 | potential |
| tiopronin | 0.00 | 1795 | potential |
| CAY10505 | 0.00 | 1796 | potential |
| IBOA-(DL) | 0.00 | 1797 | potential |
| tiracizine | 0.00 | 1798 | potential |
| fluvastatin | 0.00 | 1799 | potential |
| propoxycaine | 0.00 | 1800 | potential |
| CGP-53353 | 0.00 | 1801 | potential |
| dromostanolone-propionate | 0.00 | 1802 | potential |
| galeterone | 0.00 | 1803 | potential |
| CGP-52411 | 0.00 | 1804 | potential |
| doxazosin | 0.00 | 1805 | potential |
| GSK3787 | 0.00 | 1806 | potential |
| alvocidib | 0.00 | 1807 | potential |
| acedapsone | 0.00 | 1808 | potential |
| PF-02545920 | 0.00 | 1809 | potential |
| tecalcet | 0.00 | 1810 | potential |
| EPZ004777 | 0.00 | 1811 | potential |
| metaxalone | 0.00 | 1812 | potential |
| griseofulvin | 0.00 | 1813 | potential |
| D-Phenylalanine | 0.00 | 1814 | potential |
| tetradecylthioacetic-acid | 0.00 | 1815 | potential |
| loredipion | 0.00 | 1816 | potential |
| bromfenac | 0.00 | 1817 | potential |
| carisoprodol | 0.00 | 1818 | potential |
| cholic-acid | 0.00 | 1819 | potential |
| estropipate | 0.00 | 1820 | potential |
| polythiazide | 0.00 | 1821 | potential |
| N-acetyl-mannosamine | 0.00 | 1822 | potential |
| diminazene-aceturate | 0.00 | 1823 | potential |
| SRC-kinase-inhibitor-I | 0.00 | 1824 | potential |
| PD-153035 | 0.00 | 1825 | potential |
| 3-alpha-bis-(4-fluorophenyl)- | 0.00 | 1826 | potential |
| methoxytropane | | | |
| rifampin | 0.00 | 1827 | potential |
| AS-77 | 0.00 | 1828 | potential |
| bezafibrate | 0.00 | 1829 | potential |
| JNJ-38877605 | 0.00 | 1830 | potential |
| SC-10 | 0.00 | 1831 | potential |
| penicillamine-(D) | 0.00 | 1832 | potential |
| dexloxiglumide | 0.00 | 1833 | potential |
| carbinoxamine | 0.00 | 1834 | potential |
| tamibarotene | 0.00 | 1835 | potential |
| immepip | 0.00 | 1836 | potential |
| cyanopindolol | 0.00 | 1837 | potential |
| 2-iminobiotin | 0.00 | 1838 | potential |
| arotinolol | 0.00 | 1839 | potential |
| TAK-901 | 0.00 | 1840 | potential |
| terutroban | 0.00 | 1841 | potential |
| SDM25N | 0.00 | 1842 | potential |
| tie2-kinase-inhibitor | 0.00 | 1843 | potential |
| lacitol | 0.00 | 1844 | potential |
| citiolone | 0.00 | 1845 | potential |
| BIX-02188 | 0.00 | 1846 | potential |
| isbufylline | 0.00 | 1847 | potential |
| ditolylguanidine | 0.00 | 1848 | potential |
| clevudine | 0.00 | 1849 | potential |
| DMP-543 | 0.00 | 1850 | potential |
| KB-R7943 | 0.00 | 1851 | potential |
| pimethixene | 0.00 | 1852 | potential |
| adapalene | 0.00 | 1853 | potential |
| venlafaxine | 0.00 | 1854 | potential |
| piroximone | 0.00 | 1855 | potential |
| BU-239 | 0.00 | 1856 | potential |
| NU-1025 | 0.00 | 1857 | potential |
| SB-742457 | 0.00 | 1858 | potential |
| lemborexant | 0.00 | 1859 | potential |
| nornicotine | 0.00 | 1860 | potential |
| SKF-89976A | 0.00 | 1861 | potential |
| salicylanilide | 0.00 | 1862 | potential |
| bardoxolone-methyl | 0.00 | 1863 | potential |
| clinafloxacin | 0.00 | 1864 | potential |
| nefopam | 0.00 | 1865 | potential |
| SU014813 | 0.00 | 1866 | potential |
| rivastigmine | 0.00 | 1867 | potential |
| igmesine | 0.00 | 1868 | potential |
| tuaminoheptane | 0.00 | 1869 | potential |
| chlorindione | 0.00 | 1870 | potential |
| trifluoperazine | 0.00 | 1871 | potential |
| mizolastine | 0.00 | 1872 | potential |
| phenacetin | 0.00 | 1873 | potential |
| fulvestrant | 0.00 | 1874 | potential |
| CP-93129 | 0.00 | 1875 | potential |
| suloctidil | 0.00 | 1876 | potential |
| bopindolol | 0.00 | 1877 | potential |
| pifithrin-mu | 0.00 | 1878 | potential |
| HA-1004 | 0.00 | 1879 | potential |
| danoprevir | 0.00 | 1880 | potential |
| DIPT | 0.00 | 1881 | potential |
| GS-39783 | 0.00 | 1882 | potential |
| valethamate | 0.00 | 1883 | potential |
| hexoprenaline | 0.00 | 1884 | potential |
| SNX-2112 | 0.00 | 1885 | potential |
| ecabet | 0.00 | 1886 | potential |
| L-798,106 | 0.00 | 1887 | potential |
| calcipotriol | 0.00 | 1888 | potential |
| cobicistat | 0.00 | 1889 | potential |
| diazepam | 0.00 | 1890 | potential |
| pentobarbital | 0.00 | 1891 | potential |
| chrysin | 0.00 | 1892 | potential |
| paroxypropione | 0.00 | 1893 | potential |
| bacampicillin | 0.00 | 1894 | potential |
| AMG-208 | 0.00 | 1895 | potential |
| elesclomol | 0.00 | 1896 | potential |
| clodronic-acid | 0.00 | 1897 | potential |
| BMS-777607 | 0.00 | 1898 | potential |
| NNC-63-0532 | 0.00 | 1899 | potential |
| A-33903 | 0.00 | 1900 | potential |
| raclopride | 0.00 | 1901 | potential |
| zaleplon | 0.00 | 1902 | potential |
| AFN-1252 | 0.00 | 1903 | potential |
| ST-1859 | 0.00 | 1904 | potential |
| BRL-52537 | 0.00 | 1905 | potential |
| pemetrexed | 0.00 | 1906 | potential |
| metergoline | 0.00 | 1907 | potential |
| folic-acid | 0.00 | 1908 | potential |
| bifemelane | 0.00 | 1909 | potential |
| eprosartan | 0.00 | 1910 | potential |
| cephalothin | 0.00 | 1911 | potential |
| amoxicillin | 0.00 | 1912 | potential |
| dexamethasone-sodium-phosphate | 0.00 | 1913 | potential |
| propentofylline | 0.00 | 1914 | potential |
| icatibant-acetate | 0.00 | 1915 | potential |
| avagacestat | 0.00 | 1916 | potential |
| ergocalciferol | 0.00 | 1917 | potential |
| floxuridine | 0.00 | 1918 | potential |
| clobetasone-butyrate | 0.00 | 1919 | potential |
| AZ191 | 0.00 | 1920 | potential |
| econazole | 0.00 | 1921 | potential |
| torasemide | 0.00 | 1922 | potential |
| lazabemide | 0.00 | 1923 | potential |
| mepenzolate | 0.00 | 1924 | potential |
| 4-acetyl-1,1-dimethylpiperazinium | 0.00 | 1925 | potential |
| 2-thiouracil | 0.00 | 1926 | potential |
| ADX-47273 | 0.00 | 1927 | potential |
| metolazone | 0.00 | 1928 | potential |
| salvianolic-acid-B | 0.00 | 1929 | potential |
| spironolactone | 0.00 | 1930 | potential |
| emorfazone | 0.00 | 1931 | potential |
| carbetocin | 0.00 | 1932 | potential |
| camicinal | 0.00 | 1933 | potential |
| gentiopicrin | 0.00 | 1934 | potential |
| ATN-161 | 0.00 | 1935 | potential |
| 3-deazaneplanocin-A | 0.00 | 1936 | potential |
| L-Cysteinesulfinic-acid | 0.00 | 1937 | potential |
| nicotinyl-alcohol-tartrate | 0.00 | 1938 | potential |
| cloprostenol-(+/−) | 0.00 | 1939 | potential |
| zafirlukast | 0.00 | 1940 | potential |
| dichlorisone-acetate | 0.00 | 1941 | potential |
| ZD-2079 | 0.00 | 1942 | potential |
| SCMC-Lys | 0.00 | 1943 | potential |
| sodium-danshensu | 0.00 | 1944 | potential |
| cefcapene-pivoxil | 0.00 | 1945 | potential |
| acebutolol | 0.00 | 1946 | potential |
| isoxicam | 0.00 | 1947 | potential |
| Dexchlor-pheniramine | 0.00 | 1948 | potential |
| benserazide | 0.00 | 1949 | potential |
| secoisolariciresinol-diglucoside | 0.00 | 1950 | potential |
| teriflunomide | 0.00 | 1951 | potential |
| UNC0638 | 0.00 | 1952 | potential |
| CZC24832 | 0.00 | 1953 | potential |
| pridinol | 0.00 | 1954 | potential |
| STF-118804 | 0.00 | 1955 | potential |
| histamine | 0.00 | 1956 | potential |
| INH1 | 0.00 | 1957 | potential |
| Trimethobenzamide | 0.00 | 1958 | potential |
| vortioxetine | 0.00 | 1959 | potential |
| acetyl-farnesyl-cysteine | 0.00 | 1960 | potential |
| antazoline | 0.00 | 1961 | potential |
| talarozole | 0.00 | 1962 | potential |
| CGH2466 | 0.00 | 1963 | potential |
| IU1 | 0.00 | 1964 | potential |
| 1400W | 0.00 | 1965 | potential |
| L-glutamic-acid | 0.00 | 1966 | potential |
| azlocillin | 0.00 | 1967 | potential |
| zofenopril-calcium | 0.00 | 1968 | potential |
| bisacodyl | 0.00 | 1969 | potential |
| bakuchiol | 0.00 | 1970 | potential |
| licarbazepine | 0.00 | 1971 | potential |
| hesperetin | 0.00 | 1972 | potential |
| flurbiprofen-(+/−) | 0.00 | 1973 | potential |
| cilazapril | 0.00 | 1974 | potential |
| capsaicin | 0.00 | 1975 | potential |
| finasteride | 0.00 | 1976 | potential |
| lactulose | 0.00 | 1977 | potential |
| A66 | 0.00 | 1978 | potential |
| orlistat | 0.00 | 1979 | potential |
| GW-583340 | 0.00 | 1980 | potential |
| dapagliflozin | 0.00 | 1981 | potential |
| tioconazole | 0.00 | 1982 | potential |
| erythromycin | 0.00 | 1983 | potential |
| dosulepin | 0.00 | 1984 | potential |
| BRL-54443 | 0.00 | 1985 | potential |
| L-methionine | 0.00 | 1986 | potential |
| nedaplatin | 0.00 | 1987 | potential |
| fenofibric-acid | 0.00 | 1988 | potential |
| maprotiline | 0.00 | 1989 | potential |
| S-111 | 0.00 | 1990 | potential |
| topiroxostat | 0.00 | 1991 | potential |
| EXO-1 | 0.00 | 1992 | potential |
| potassium-canrenoate | 0.00 | 1993 | potential |
| daprodustat | 0.00 | 1994 | potential |
| hymecromone | 0.00 | 1995 | potential |
| cinflumide | 0.00 | 1996 | potential |
| cyproterone-acetate | 0.00 | 1997 | potential |
| mebrofenin | 0.00 | 1998 | potential |
| lorazepam | 0.00 | 1999 | potential |
| EVP-6124 | 0.00 | 2000 | potential |
| bremelanotide | 0.00 | 2001 | potential |
| AZD2014 | 0.00 | 2002 | potential |
| bemegride | 0.00 | 2003 | potential |
| olsalazine | 0.00 | 2004 | potential |
| oxyphenonium | 0.00 | 2005 | potential |
| lacidipine | 0.00 | 2006 | potential |
| chlorzoxazone | 0.00 | 2007 | potential |
| endo-IWR-1 | 0.00 | 2008 | potential |
| diloxanide | 0.00 | 2009 | potential |
| moxaverine | 0.00 | 2010 | potential |
| naltrindole | 0.00 | 2011 | potential |
| sucrose-octaacetate | 0.00 | 2012 | potential |
| PF-04691502 | 0.00 | 2013 | potential |
| ketoprofen | 0.00 | 2014 | potential |
| moracizine | 0.00 | 2015 | potential |
| tripelennamine | 0.00 | 2016 | potential |
| indobufen | 0.00 | 2017 | potential |
| FK-33-824 | 0.00 | 2018 | potential |
| frovatriptan | 0.00 | 2019 | potential |
| setipiprant | 0.00 | 2020 | potential |
| Ro-25-6981 | 0.00 | 2021 | potential |
| cytochalasin-b | 0.00 | 2022 | potential |
| proscillaridin-a | 0.00 | 2023 | potential |
| aclidinium | 0.00 | 2024 | potential |
| EPZ-5676 | 0.00 | 2025 | potential |
| etofibrate | 0.00 | 2026 | potential |
| siponimod | 0.00 | 2027 | potential |
| milacemide | 0.00 | 2028 | potential |
| mabuprofen | 0.00 | 2029 | potential |
| GW-3965 | 0.00 | 2030 | potential |
| rifaximin | 0.00 | 2031 | potential |
| L-655,708 | 0.00 | 2032 | potential |
| pirlindole | 0.00 | 2033 | potential |
| lincomycin | 0.00 | 2034 | potential |
| nifenazone | 0.00 | 2035 | potential |
| SKF-96365 | 0.00 | 2036 | potential |
| pimonidazole | 0.00 | 2037 | potential |
| scopolamine | 0.00 | 2038 | potential |
| 8-hydroxy-PIPAT | 0.00 | 2039 | potential |
| carbenoxolone | 0.00 | 2040 | potential |
| mefloquine | 0.00 | 2041 | potential |
| PU-H71 | 0.00 | 2042 | potential |
| diatrizoic-acid | 0.00 | 2043 | potential |
| guvacine | 0.00 | 2044 | potential |
| exalamide | 0.00 | 2045 | potential |
| Hydroxylchloroquine | 0.00 | 2046 | potential |
| liothyronine-(isomer) | 0.00 | 2047 | potential |
| lubiprostone | 0.00 | 2048 | potential |
| SAR131675 | 0.00 | 2049 | potential |
| isosorbide-mononitrate | 0.00 | 2050 | potential |
| sofalcone | 0.00 | 2051 | potential |
| spiramycin | 0.00 | 2052 | potential |
| ascorbic-acid | 0.00 | 2053 | potential |
| isovaleramide | 0.00 | 2054 | potential |
| quinapril | 0.00 | 2055 | potential |
| ranitidine | 0.00 | 2056 | potential |
| nicardipine | 0.00 | 2057 | potential |
| donitriptan | 0.00 | 2058 | potential |
| sa1003 | 0.00 | 2059 | potential |
| droloxifene | 0.00 | 2060 | potential |
| SB-239063 | 0.00 | 2061 | potential |
| estrone | 0.00 | 2062 | potential |
| azosemide | 0.00 | 2063 | potential |
| JTE-907 | 0.00 | 2064 | potential |
| propacetamol | 0.00 | 2065 | potential |
| SM-108 | 0.00 | 2066 | potential |
| resorcinol | 0.00 | 2067 | potential |
| hydroxyprogesterone-acetate | 0.00 | 2068 | potential |
| piboserod | 0.00 | 2069 | potential |
| ramifenazone | 0.00 | 2070 | potential |
| tipranavir | 0.00 | 2071 | potential |
| 17-alpha-methyltestosterone | 0.00 | 2072 | potential |
| pilocarpine | 0.00 | 2073 | potential |
| pentagastrin | 0.00 | 2074 | potential |
| Ethylenediaminetetraaceric-acid | 0.00 | 2075 | potential |
| emoxipin | 0.00 | 2076 | potential |
| crisaborole | 0.00 | 2077 | potential |
| tiabendazole | 0.00 | 2078 | potential |
| naratriptan | 0.00 | 2079 | potential |
| rizatriptan | 0.00 | 2080 | potential |
| vanillin | 0.00 | 2081 | potential |
| octisalate | 0.00 | 2082 | potential |
| natamycin | 0.00 | 2083 | potential |
| CX-4945 | 0.00 | 2084 | potential |
| danegaptide | 0.00 | 2085 | potential |
| motesanib | 0.00 | 2086 | potential |
| VX-745 | 0.00 | 2087 | potential |
| ioversol | 0.00 | 2088 | potential |
| ethinyl-estradiol | 0.00 | 2089 | potential |
| gabexate | 0.00 | 2090 | potential |
| pranoprofen | 0.00 | 2091 | potential |
| vicriviroc | 0.00 | 2092 | potential |
| SNC-80 | 0.00 | 2093 | potential |
| BMS-833923 | 0.00 | 2094 | potential |
| metyrapone | 0.00 | 2095 | potential |
| montelukast | 0.00 | 2096 | potential |
| LY2452473 | 0.00 | 2097 | potential |
| cresol | 0.00 | 2098 | potential |
| colesevalam | 0.00 | 2099 | potential |
| lamivudine | 0.00 | 2100 | potential |
| oseltamivir-carboxylate | 0.00 | 2101 | potential |
| benzydamine | 0.00 | 2102 | potential |
| SYM-2081 | 0.00 | 2103 | potential |
| FG-7142 | 0.00 | 2104 | potential |
| tiprenolol | 0.00 | 2105 | potential |
| UK-383367 | 0.00 | 2106 | potential |
| GDC-0810 | 0.00 | 2107 | potential |
| phenacemide | 0.00 | 2108 | potential |
| sodium-tetradecyl-sulfate | 0.00 | 2109 | potential |
| boronophenylalanine | 0.00 | 2110 | potential |
| dicloxacillin | 0.00 | 2111 | potential |
| PS178990 | 0.00 | 2112 | potential |
| metocurine | 0.00 | 2113 | potential |
| 2-amino-benzenesulfonamide | 0.00 | 2114 | potential |
| sinomenine | 0.00 | 2115 | potential |
| eperisone | 0.00 | 2116 | potential |
| PD-160170 | 0.00 | 2117 | potential |
| closantel | 0.00 | 2118 | 0 |
| piceatannol | 0.00 | 2119 | 0 |
| capecitabine | 0.00 | 2120 | 0 |
| glycitein | 0.00 | 2121 | 0 |
| tacrolimus | 0.00 | 2122 | 0 |
| SB-705498 | 0.00 | 2123 | 0 |
| cabaletta | 0.00 | 2124 | 0 |
| naltrexone | 0.00 | 2125 | 0 |
| cefotiam | 0.00 | 2126 | 0 |
| travoprost | 0.00 | 2127 | 0 |
| arecaidine-but-2-ynyl-ester | 0.00 | 2128 | 0 |
| 6-iodo-nordihydrocapsaicin | 0.00 | 2129 | 0 |
| caffeic-acid-phenethyl-ester | 0.00 | 2130 | 0 |
| BQ-123 | 0.00 | 2131 | 0 |
| flurbiprofen-(S)-(+) | 0.00 | 2132 | 0 |
| trans-4-[8-(3-Fluorophenyl)- | 0.00 | 2133 | 0 |
| 1,7-naphthyridin-6- | | | |
| yl]cyclohexanecarboxylie-acid | | | |
| 7-hydroxy-staurosporine | 0.00 | 2134 | 0 |
| moprolol | 0.00 | 2135 | 0 |
| asymmetrical-dimethylarginine | 0.00 | 2136 | 0 |
| zuclopenthixol | 0.00 | 2137 | 0 |
| albuterol | 0.00 | 2138 | 0 |
| naloxegol | 0.00 | 2139 | 0 |
| amlodipine | 0.00 | 2140 | 0 |
| bethanechol | 0.00 | 2141 | 0 |
| SKF-38393 | 0.00 | 2142 | 0 |
| sertraline | 0.00 | 2143 | 0 |
| ranolazine | 0.00 | 2144 | 0 |
| priralfinamide | 0.00 | 2145 | 0 |
| zibotentan | 0.00 | 2146 | 0 |
| delamanid | 0.00 | 2147 | 0 |
| DPI-201106 | 0.00 | 2148 | 0 |
| pravastatin | 0.00 | 2149 | 0 |
| pinitol | 0.00 | 2150 | 0 |
| 3-MPPI | 0.00 | 2151 | 0 |
| zaprinast | 0.00 | 2152 | 0 |
| verapamil | 0.00 | 2153 | 0 |
| scopolamine-n-oxide | 0.00 | 2154 | 0 |
| BYK-204165 | 0.00 | 2155 | 0 |
| loxistatin-acid | 0.00 | 2156 | 0 |
| oxytocin | 0.00 | 2157 | 0 |
| propofol | 0.00 | 2158 | 0 |
| ezatiostat | 0.00 | 2159 | 0 |
| bupivacaine | 0.00 | 2160 | 0 |
| allopurinol | 0.00 | 2161 | 0 |
| moguisteine | 0.00 | 2162 | 0 |
| coumophos | 0.00 | 2163 | 0 |
| terbutaline | 0.00 | 2164 | 0 |
| doxepin | 0.00 | 2165 | 0 |
| JZL-184 | 0.00 | 2166 | 0 |
| desogestrel | 0.00 | 2167 | 0 |
| N20C | 0.00 | 2168 | 0 |
| N-methyl-(−)ephedrine-[1r,2s] | 0.00 | 2169 | 0 |
| AR-12 | 0.00 | 2170 | 0 |
| deracoxib | 0.00 | 2171 | 0 |
| Methyllycaconitine | 0.00 | 2172 | 0 |
| HA-966-(R)-(+) | 0.00 | 2173 | 0 |
| L-Theanine | 0.00 | 2174 | 0 |
| tolonidine | 0.00 | 2175 | 0 |
| Ethylnorepinephrine | 0.00 | 2176 | 0 |
| TMS | 0.00 | 2177 | 0 |
| PGL5001 | 0.00 | 2178 | 0 |
| alvimopan | 0.00 | 2179 | 0 |
| rocuronium | 0.00 | 2180 | 0 |
| chiniofon | 0.00 | 2181 | 0 |
| fluoxymesterone | 0.00 | 2182 | 0 |
| succinic-acid | 0.00 | 2183 | 0 |
| cyclandelate | 0.00 | 2184 | 0 |
| TU-2100 | 0.00 | 2185 | 0 |
| UNC1999 | 0.00 | 2186 | 0 |
| cinanserin | 0.00 | 2187 | 0 |
| diethylstilbestrol | 0.00 | 2188 | 0 |
| cloranolol | 0.00 | 2189 | 0 |
| ZSTK-474 | 0.00 | 2190 | 0 |
| acexamic-acid | 0.00 | 2191 | 0 |
| Org-12962 | 0.00 | 2192 | 0 |
| fiacitabine | 0.00 | 2193 | 0 |
| levocarnitine | 0.00 | 2194 | 0 |
| methyldopa | 0.00 | 2195 | 0 |
| cinacalcet | 0.00 | 2196 | 0 |
| climbazole | 0.00 | 2197 | 0 |
| tiletamine | 0.00 | 2198 | 0 |
| ramatroban | 0.00 | 2199 | 0 |
| ruxolitinib | 0.00 | 2200 | 0 |
| 2-iodomelatonin | 0.00 | 2201 | 0 |
| estradiol-acetate | 0.00 | 2202 | 0 |
| hemoglobin-modulators-1 | 0.00 | 2203 | 0 |
| vildagliptin | 0.00 | 2204 | 0 |
| linopirdine | 0.00 | 2205 | 0 |
| pimobendan | 0.00 | 2206 | 0 |
| N-[2-(Piperidinylamino)ethyl]- | 0.00 | 2207 | 0 |
| 4-iodobenzamide | | | |
| melatonin | 0.00 | 2208 | 0 |
| protriptyline | 0.00 | 2209 | 0 |
| cromakalim | 0.00 | 2210 | 0 |
| oleanolic-acid | 0.00 | 2211 | 0 |
| betaxolol | 0.00 | 2212 | 0 |
| dioxybenzone | 0.00 | 2213 | 0 |
| sodium-picosulfate | 0.00 | 2214 | 0 |
| afloqualone | 0.00 | 2215 | 0 |
| GW-501516 | 0.00 | 2216 | 0 |
| oxyfedrine | 0.00 | 2217 | 0 |
| pimozide | 0.00 | 2218 | 0 |
| trans-10,cis-12-Conjugated- | 0.00 | 2219 | 0 |
| linoleic-acid | | | |
| azimilide | 0.00 | 2220 | 0 |
| mecarbinate | 0.00 | 2221 | 0 |
| NPC-01 | 0.00 | 2222 | 0 |
| cinepazide | 0.00 | 2223 | 0 |
| dihydrostreptomycin | 0.00 | 2224 | 0 |
| arecoline | 0.00 | 2225 | 0 |
| sulfacarbamide | 0.00 | 2226 | 0 |
| camptothecin | 0.00 | 2227 | 0 |
| troxerutin | 0.00 | 2228 | 0 |
| sodium-butyrate | 0.00 | 2229 | 0 |
| RSV604 | 0.00 | 2230 | 0 |
| lisofylline | 0.00 | 2231 | 0 |
| thiamet-g | 0.00 | 2232 | 0 |
| hydroxyurea | 0.00 | 2233 | 0 |
| lirimilast | 0.00 | 2234 | 0 |
| apraclonidine | 0.00 | 2235 | 0 |
| hemomex-s | 0.00 | 2236 | 0 |
| pevonedistat | 0.00 | 2237 | 0 |
| CHF5074 | 0.00 | 2238 | 0 |
| acetohexamide | 0.00 | 2239 | 0 |
| SR-59230A | 0.00 | 2240 | 0 |
| khellin | 0.00 | 2241 | 0 |
| alcuronium | 0.00 | 2242 | 0 |
| pyronaridine | 0.00 | 2243 | 0 |
| yohimbine | 0.00 | 2244 | 0 |
| pyrazolanthrone | 0.00 | 2245 | 0 |
| ZD-7114 | 0.00 | 2246 | 0 |
| SC-19220 | 0.00 | 2247 | 0 |
| solifenacin-succinate | 0.00 | 2248 | 0 |
| tramadol | 0.00 | 2249 | 0 |
| PF-5274857 | 0.00 | 2250 | 0 |
| LXR-623 | 0.00 | 2251 | 0 |
| zotarolimus | 0.00 | 2252 | 0 |
| SN-6 | 0.00 | 2253 | 0 |
| LY2784544 | 0.00 | 2254 | 0 |
| ceftiofur | 0.00 | 2255 | 0 |
| strychnine | 0.00 | 2256 | 0 |
| isoxepac | 0.00 | 2257 | 0 |
| pikamilone | 0.00 | 2258 | 0 |
| etomidate | 0.00 | 2259 | 0 |
| actarit | 0.00 | 2260 | 0 |
| hydroxypropyl-beta-cyclodextrin | 0.00 | 2261 | 0 |
| PD-173074 | 0.00 | 2262 | 0 |
| gaboxadol | 0.00 | 2263 | 0 |
| piperaquine-phosphate | 0.00 | 2264 | 0 |
| 4-iodo-L-phenylalanine | 0.00 | 2265 | 0 |
| 2-TEDC | 0.00 | 2266 | 0 |
| etosalamide | 0.00 | 2267 | 0 |
| procainamide | 0.00 | 2268 | 0 |
| methylatropine-nitrate | 0.00 | 2269 | 0 |
| physostigmine | 0.00 | 2270 | 0 |
| ivabradine | 0.00 | 2271 | 0 |
| tempol | 0.00 | 2272 | 0 |
| desoximetasone | 0.00 | 2273 | 0 |
| citicoline | 0.00 | 2274 | 0 |
| enprofylline | 0.00 | 2275 | 0 |
| cevimeline | 0.00 | 2276 | 0 |
| bicuculline-methochloride-(−) | 0.00 | 2277 | 0 |
| ciproxifan | 0.00 | 2278 | 0 |
| SQ-22536 | 0.00 | 2279 | 0 |
| betaine | 0.00 | 2280 | 0 |
| Ro-15-4513 | 0.00 | 2281 | 0 |
| oxolinic-acid | 0.00 | 2282 | 0 |
| sulmetozine | 0.00 | 2283 | 0 |
| vonoprazan | 0.00 | 2284 | 0 |
| sodium-monofluorophosphate | 0.00 | 2285 | 0 |
| apratastat | 0.00 | 2286 | 0 |
| terodiline | 0.00 | 2287 | 0 |
| U-0124 | 0.00 | 2288 | 0 |
| Capecitabine | 0.00 | 2289 | 0 |
| prinaberel | 0.00 | 2290 | 0 |
| iniparib | 0.00 | 2291 | 0 |
| dimethindene-(S)-(+) | 0.00 | 2292 | 0 |
| methsuximide | 0.00 | 2293 | 0 |
| bretazenil | 0.00 | 2294 | 0 |
| AZD2461 | 0.00 | 2295 | 0 |
| N-methylquipazine | 0.00 | 2296 | 0 |
| NVP-TAE226 | 0.00 | 2297 | 0 |
| chlorphensin-carbamate | 0.00 | 2298 | 0 |
| thiothixene | 0.00 | 2299 | 0 |
| nintedanib | 0.00 | 2300 | 0 |
| riboflavin | 0.00 | 2301 | 0 |
| lenvatinib | 0.00 | 2302 | 0 |
| ethisterone | 0.00 | 2303 | 0 |
| anamorelin | 0.00 | 2304 | 0 |
| GSK1838705A | 0.00 | 2305 | 0 |
| amthamine | 0.00 | 2306 | 0 |
| entrectinib | 0.00 | 2307 | 0 |
| AM-1241 | 0.00 | 2308 | 0 |
| RS-23597-190 | 0.00 | 2309 | 0 |
| talipexole | 0.00 | 2310 | 0 |
| AS-1949490 | 0.00 | 2311 | 0 |
| buflomedil | 0.00 | 2312 | 0 |
| cis-9,trans-11-Conjugated- | 0.00 | 2313 | 0 |
| linoleic-acid | | | |
| MDL-73005EF | 0.00 | 2314 | 0 |
| sulfadiazine | 0.00 | 2315 | 0 |
| ethotoin | 0.00 | 2316 | 0 |
| abametapir | 0.00 | 2317 | 0 |
| fidaxomicin | 0.00 | 2318 | 0 |
| ceftriaxone | 0.00 | 2319 | 0 |
| tosufloxacin | 0.00 | 2320 | 0 |
| EMD-66684 | 0.00 | 2321 | 0 |
| tofisopam | 0.00 | 2322 | 0 |
| nilutamide | 0.00 | 2323 | 0 |
| eliprodil | 0.00 | 2324 | 0 |
| zilpaterol | 0.00 | 2325 | 0 |
| silibinin | 0.00 | 2326 | 0 |
| adoprazine | 0.00 | 2327 | 0 |
| LY255283 | 0.00 | 2328 | 0 |
| maxacalcitol | 0.00 | 2329 | 0 |
| AGN-194310 | 0.00 | 2330 | 0 |
| phenylacetylglutamine | 0.00 | 2331 | 0 |
| menadione-bisulfite | 0.00 | 2332 | 0 |
| tolcapone | 0.00 | 2333 | 0 |
| 8-bromo-cAMP | 0.00 | 2334 | 0 |
| PRX-08066 | 0.00 | 2335 | 0 |
| PMPA | 0.00 | 2336 | 0 |
| sulfaguanidine | 0.00 | 2337 | 0 |
| IPPS4 | 0.00 | 2338 | 0 |
| telbivudine | 0.00 | 2339 | 0 |
| LTB4 | 0.00 | 2340 | 0 |
| arsanilic-acid | 0.00 | 2341 | 0 |
| uric-acid | 0.00 | 2342 | 0 |
| chenodeoxycholic-acid | 0.00 | 2343 | 0 |
| disopyramide | 0.00 | 2344 | 0 |
| SB-242235 | 0.00 | 2345 | 0 |
| terbinafine | 0.00 | 2346 | 0 |
| sulmazole | 0.00 | 2347 | 0 |
| roxarsone | 0.00 | 2348 | 0 |
| bazedoxifene | 0.00 | 2349 | 0 |
| XBD173 | 0.00 | 2350 | 0 |
| molidustat | 0.00 | 2351 | 0 |
| doxofylline | 0.00 | 2352 | 0 |
| reserpine | 0.00 | 2353 | 0 |
| AMN-082 | 0.00 | 2354 | 0 |
| dimesna | 0.00 | 2355 | 0 |
| erdosteine | 0.00 | 2356 | 0 |
| streptozotocin | 0.00 | 2357 | 0 |
| clobazam | 0.00 | 2358 | 0 |
| raltitrexed | 0.00 | 2359 | 0 |
| formestane | 0.00 | 2360 | 0 |
| 3-(4-methylbenzylidene)camphor | 0.00 | 2361 | 0 |
| carbenoxolone | 0.00 | 2362 | 0 |
| VCH-916 | 0.00 | 2363 | 0 |
| sivelestat | 0.00 | 2364 | 0 |
| tyrphostin-AG-18 | 0.00 | 2365 | 0 |
| methylnaltrexone | 0.00 | 2366 | 0 |
| nicergoline | 0.00 | 2367 | 0 |
| MK-0354 | 0.00 | 2368 | 0 |
| fluoxetine | 0.00 | 2369 | 0 |
| S18986 | 0.00 | 2370 | 0 |
| degarelix | 0.00 | 2371 | 0 |
| edoxudine | 0.00 | 2372 | 0 |
| SQ-109 | 0.00 | 2373 | 0 |
| primaquine | 0.00 | 2374 | 0 |
| althiazide | 0.00 | 2375 | 0 |
| GGTI-298 | 0.00 | 2376 | 0 |
| rolipram-R(−) | 0.00 | 2377 | 0 |
| swainsonine | 0.00 | 2378 | 0 |
| troleandomycin | 0.00 | 2379 | 0 |
| phenylephrine | 0.00 | 2380 | 0 |
| VU0357121 | 0.00 | 2381 | 0 |
| troglitazone | 0.00 | 2382 | 0 |
| methocarbamol | 0.00 | 2383 | 0 |
| IOWH032 | 0.00 | 2384 | 0 |
| fluocinonide | 0.00 | 2385 | 0 |
| RS-102221 | 0.00 | 2386 | 0 |
| pivmecillinam | 0.00 | 2387 | 0 |
| nicotine | 0.00 | 2388 | 0 |
| ursodeoxycholyltaurine | 0.00 | 2389 | 0 |
| bambuterol | 0.00 | 2390 | 0 |
| talampanel | 0.00 | 2391 | 0 |
| anacetrapib | 0.00 | 2392 | 0 |
| hydrastinine | 0.00 | 2393 | 0 |
| amylene-hydrate | 0.00 | 2394 | 0 |
| cefoselis | 0.00 | 2395 | 0 |
| desoxycorticosterone-pivalate | 0.00 | 2396 | 0 |
| aminosalicylate | 0.00 | 2397 | 0 |
| ETP-46464 | 0.00 | 2398 | 0 |
| mebhydrolin | 0.00 | 2399 | 0 |
| traxoprodil | 0.00 | 2400 | 0 |
| 3,3′-dichlorobenzaldazine | 0.00 | 2401 | 0 |
| octinoxate | 0.00 | 2402 | 0 |
| nithiamide | 0.00 | 2403 | 0 |
| pleconaril | 0.00 | 2404 | 0 |
| aminosalicylate | 0.00 | 2405 | 0 |
| norelgestromin | 0.00 | 2406 | 0 |
| rimantadine | 0.00 | 2407 | 0 |
| tetracycline | 0.00 | 2408 | 0 |
| azelastine | 0.00 | 2409 | 0 |
| dihydrotestosterone | 0.00 | 2410 | 0 |
| indoprofen | 0.00 | 2411 | 0 |
| erythrosine | 0.00 | 2412 | 0 |
| sulforaphane | 0.00 | 2413 | 0 |
| adarotene | 0.00 | 2414 | 0 |
| sulfamonomethoxine | 0.00 | 2415 | 0 |
| dibutyl-phthalate | 0.00 | 2416 | 0 |
| cutamesine | 0.00 | 2417 | 0 |
| org-26576 | 0.00 | 2418 | 0 |
| hesperidin | 0.00 | 2419 | 0 |
| naltriben | 0.00 | 2420 | 0 |
| tinidazole | 0.00 | 2421 | 0 |
| chromanol-293B-(−)-[3R,4S] | 0.00 | 2422 | 0 |
| NCS-382 | 0.00 | 2423 | 0 |
| spiperone | 0.00 | 2424 | 0 |
| fomepizole | 0.00 | 2425 | 0 |
| lisinopril | 0.00 | 2426 | 0 |
| lisinopril | 0.00 | 2427 | 0 |
| betrixaban | 0.00 | 2428 | 0 |
| A740003 | 0.00 | 2429 | 0 |
| exemestane | 0.00 | 2430 | 0 |
| fidarestat | 0.00 | 2431 | 0 |
| APD668 | 0.00 | 2432 | 0 |
| cefpiramide | 0.00 | 2433 | 0 |
| levomequitazine | 0.00 | 2434 | 0 |
| CV-1808 | 0.00 | 2435 | 0 |
| molsidomine | 0.00 | 2436 | 0 |
| relebactam | 0.00 | 2437 | 0 |
| dideoxyadenosine | 0.00 | 2438 | 0 |
| bupropion | 0.00 | 2439 | 0 |
| morin | 0.00 | 2440 | 0 |
| sphingosylphosphorylcholine | 0.00 | 2441 | 0 |
| zometapine | 0.00 | 2442 | 0 |
| testosterone-propionate | 0.00 | 2443 | 0 |
| cetirizine | 0.00 | 2444 | 0 |
| cintriamide | 0.00 | 2445 | 0 |
| carazolol | 0.00 | 2446 | 0 |
| tranilast | 0.00 | 2447 | 0 |
| GSK2190915 | 0.00 | 2448 | 0 |
| SANT-1 | 0.00 | 2449 | 0 |
| cridanimod | 0.00 | 2450 | 0 |
| DMNB | 0.00 | 2451 | 0 |
| L-Citrulline | 0.00 | 2452 | 0 |
| BW-373U86 | 0.00 | 2453 | 0 |
| armodafinil | 0.00 | 2454 | 0 |
| PYM50028 | 0.00 | 2455 | 0 |
| tyloxapol | 0.00 | 2456 | 0 |
| biotin | 0.00 | 2457 | 0 |
| benzthiazide | 0.00 | 2458 | 0 |
| diacerein | 0.00 | 2459 | 0 |
| MD-920 | 0.00 | 2460 | 0 |
| deoxycorticosterone-acetate | 0.00 | 2461 | 0 |
| cefoperazone | 0.00 | 2462 | 0 |
| ZLN005 | 0.00 | 2463 | 0 |
| caroverine | 0.00 | 2464 | 0 |
| neratinib | 0.00 | 2465 | 0 |
| sclareolide | 0.00 | 2466 | 0 |
| vismodegib | 0.00 | 2467 | 0 |
| filgotinib | 0.00 | 2468 | 0 |
| zomepirac | 0.00 | 2469 | 0 |
| otamixaban | 0.00 | 2470 | 0 |
| mesulergine | 0.00 | 2471 | 0 |
| chloroprocaine | 0.00 | 2472 | 0 |
| abacavir | 0.00 | 2473 | 0 |
| linsidomine | 0.00 | 2474 | 0 |
| vinpocetine | 0.00 | 2475 | 0 |
| arcyriaflavin-a | 0.00 | 2476 | 0 |
| BMS-191011 | 0.00 | 2477 | 0 |
| nepafenac | 0.00 | 2478 | 0 |
| ursodiol | 0.00 | 2479 | 0 |
| 6-benzylaminopurine | 0.00 | 2480 | 0 |
| SM-21 | 0.00 | 2481 | 0 |
| pirinixic-acid | 0.00 | 2482 | 0 |
| simvastatin | 0.00 | 2483 | 0 |
| eplerenone | 0.00 | 2484 | 0 |
| sibutramine | 0.00 | 2485 | 0 |
| 2′-MeCCPA | 0.00 | 2486 | 0 |
| N-salicoylaminophenol | 0.00 | 2487 | 0 |
| etazolate | 0.00 | 2488 | 0 |
| enalapril | 0.00 | 2489 | 0 |
| Ro-60-0175 | 0.00 | 2490 | 0 |
| bruceantin | 0.00 | 2491 | 0 |
| clorotepine | 0.00 | 2492 | 0 |
| GW-788388 | 0.00 | 2493 | 0 |
| azatadine | 0.00 | 2494 | 0 |
| pentagastrin | 0.00 | 2495 | 0 |
| deoxyepinephrine | 0.00 | 2496 | 0 |
| ipragliflozin-1-proline | 0.00 | 2497 | 0 |
| nelarabine | 0.00 | 2498 | 0 |
| 2-BFI | 0.00 | 2499 | 0 |
| moexipril | 0.00 | 2500 | 0 |
| 3-methyladenine | 0.00 | 2501 | 0 |
| aminopurvalanol-a | 0.00 | 2502 | 0 |
| chlorobutanol | 0.00 | 2503 | 0 |
| LY344864 | 0.00 | 2504 | 0 |
| PF-4708671 | 0.00 | 2505 | 0 |
| bufexamac | 0.00 | 2506 | 0 |
| cresopirine | 0.00 | 2507 | 0 |
| levodopa | 0.00 | 2508 | 0 |
| pancuronium | 0.00 | 2509 | 0 |
| diclofensine | 0.00 | 2510 | 0 |
| tropanyl-3,5-dimethylbenzoate | 0.00 | 2511 | 0 |
| SIB-1553A | 0.00 | 2512 | 0 |
| perindopril | 0.00 | 2513 | 0 |
| betazole | 0.00 | 2514 | 0 |
| puerarin | 0.00 | 2515 | 0 |
| nitenpyram | 0.00 | 2516 | 0 |
| 4-IBP | 0.00 | 2517 | 0 |
| efaproxiral | 0.00 | 2518 | 0 |
| benazepril | 0.00 | 2519 | 0 |
| opipramol | 0.00 | 2520 | 0 |
| triamcinolone | 0.00 | 2521 | 0 |
| merbarone | 0.00 | 2522 | 0 |
| bucladesine | 0.00 | 2523 | 0 |
| guaifenesin | 0.00 | 2524 | 0 |
| ufenamate | 0.00 | 2525 | 0 |
| debrisoquin | 0.00 | 2526 | 0 |
| tempol | 0.00 | 2527 | 0 |
| CNX-2006 | 0.00 | 2528 | 0 |
| RU-28318 | 0.00 | 2529 | 0 |
| ceramide | 0.00 | 2530 | 0 |
| AJ76-(+) | 0.00 | 2531 | 0 |
| ATPA | 0.00 | 2532 | 0 |
| L-733060 | 0.00 | 2533 | 0 |
| loxistatin-acid | 0.00 | 2534 | 0 |
| cyclophosphamide | 0.00 | 2535 | 0 |
| etafenone | 0.00 | 2536 | 0 |
| ML133 | 0.00 | 2537 | 0 |
| XAV-939 | 0.00 | 2538 | 0 |
| BCI-540 | 0.00 | 2539 | 0 |
| benzamil | 0.00 | 2540 | 0 |
| tolnaftate | 0.00 | 2541 | 0 |
| thiophanate | 0.00 | 2542 | 0 |
| gramine | 0.00 | 2543 | 0 |
| dyclonine | 0.00 | 2544 | 0 |
| avibactam | 0.00 | 2545 | 0 |
| phenserine | 0.00 | 2546 | 0 |
| ZK-93426 | 0.00 | 2547 | 0 |
| inimur | 0.00 | 2548 | 0 |
| pipotiazine-palmitate | 0.00 | 2549 | 0 |
| ulifloxacin | 0.00 | 2550 | 0 |
| tropicamide | 0.00 | 2551 | 0 |
| palomid-529 | 0.00 | 2552 | 0 |
| mannitol-D | 0.00 | 2553 | 0 |
| lidamidine | 0.00 | 2554 | 0 |
| fludarabine-phosphate | 0.00 | 2555 | 0 |
| cinoxate | 0.00 | 2556 | 0 |
| modaline | 0.00 | 2557 | 0 |
| aminoglutethimide | 0.00 | 2558 | 0 |
| 5-Amino-3-D- | 0.00 | 2559 | 0 |
| ribofuranosylthiazolo[4,5- | | | |
| d]pyrimidin-2,7(3H,6H)-dione | | | |
| pantethine | 0.00 | 2560 | 0 |
| ibuprofen-(s) | 0.00 | 2561 | 0 |
| halobetasol-propionate | 0.00 | 2562 | 0 |
| SDZ-205-557 | 0.00 | 2563 | 0 |
| 7-hydroxy-DPAT | 0.00 | 2564 | 0 |
| trichlormethiazide | 0.00 | 2565 | 0 |
| benperidol | 0.00 | 2566 | 0 |
| TAK-632 | 0.00 | 2567 | 0 |
| deserpidine | 0.00 | 2568 | 0 |
| SB-269970 | 0.00 | 2569 | 0 |
| alrestatin | 0.00 | 2570 | 0 |
| 4-methylhistamine | 0.00 | 2571 | 0 |
| ciprofibrate | 0.00 | 2572 | 0 |
| L-(+)-Rhamnose-Monohydrate | 0.00 | 2573 | 0 |
| tetraethylenepentamine | 0.00 | 2574 | 0 |
| indiplon | 0.00 | 2575 | 0 |
| xylazine | 0.00 | 2576 | 0 |
| fluocinolone-acetonide | 0.00 | 2577 | 0 |
| tempol | 0.00 | 2578 | 0 |
| acipimox | 0.00 | 2579 | 0 |
| bismuth-oxalate | 0.00 | 2580 | 0 |
| ceritinib | 0.00 | 2581 | 0 |
| SLV-320 | 0.00 | 2582 | 0 |
| ostarine | 0.00 | 2583 | 0 |
| AZD1981 | 0.00 | 2584 | 0 |
| fexofenadine | 0.00 | 2585 | 0 |
| GSK2330672 | 0.00 | 2586 | 0 |
| aminopentamide | 0.00 | 2587 | 0 |
| milrinone | 0.00 | 2588 | 0 |
| meptazinol | 0.00 | 2589 | 0 |
| benzoylpas | 0.00 | 2590 | 0 |
| T-0156 | 0.00 | 2591 | 0 |
| camostat-mesilate | 0.00 | 2592 | 0 |
| tocainide | 0.00 | 2593 | 0 |
| ibudilast | 0.00 | 2594 | 0 |
| MRS1845 | 0.00 | 2595 | 0 |
| tiagabine | 0.00 | 2596 | 0 |
| malotilate | 0.00 | 2597 | 0 |
| tasimelteon | 0.00 | 2598 | 0 |
| befuraline | 0.00 | 2599 | 0 |
| eglumetad | 0.00 | 2600 | 0 |
| SR-95639A | 0.00 | 2601 | 0 |
| cefadroxil | 0.00 | 2602 | 0 |
| JNJ-16259685 | 0.00 | 2603 | 0 |
| palovarotene | 0.00 | 2604 | 0 |
| setiptiline | 0.00 | 2605 | 0 |
| L-NIL | 0.00 | 2606 | 0 |
| atenolol-(−) | 0.00 | 2607 | 0 |
| 3-bromopyruvate | 0.00 | 2608 | 0 |
| enzastaurin | 0.00 | 2609 | 0 |
| R-96544 | 0.00 | 2610 | 0 |
| XL388 | 0.00 | 2611 | 0 |
| VTP-27999 | 0.00 | 2612 | 0 |
| G-15 | 0.00 | 2613 | 0 |
| nifenalol | 0.00 | 2614 | 0 |
| fenspiride | 0.00 | 2615 | 0 |
| Y-39983 | 0.00 | 2616 | 0 |
| norfloxacin | 0.00 | 2617 | 0 |
| buthionine-sulfoximine | 0.00 | 2618 | 0 |
| salubrinal | 0.00 | 2619 | 0 |
| vincamine | 0.00 | 2620 | 0 |
| epoprostenol | 0.00 | 2621 | 0 |
| guanaben-acetate | 0.00 | 2622 | 0 |
| ZK-164015 | 0.00 | 2623 | 0 |
| alverine | 0.00 | 2624 | 0 |
| fenoterol | 0.00 | 2625 | 0 |
| enisamium-iodide | 0.00 | 2626 | 0 |
| O-1918 | 0.00 | 2627 | 0 |
| ramipril | 0.00 | 2628 | 0 |
| 1-acetyl-4-methylpiperazine | 0.00 | 2629 | 0 |
| clopidol | 0.00 | 2630 | 0 |
| benzoquinonium-dibromide | 0.00 | 2631 | 0 |
| GSK-3-inhibitor-IX | 0.00 | 2632 | 0 |
| levalbuterol | 0.00 | 2633 | 0 |
| PD-102807 | 0.00 | 2634 | 0 |
| BF2.649 | 0.00 | 2635 | 0 |
| MK-2295 | 0.00 | 2636 | 0 |
| pheniramine | 0.00 | 2637 | 0 |
| ML347 | 0.00 | 2638 | 0 |
| SRT2104 | 0.00 | 2639 | 0 |
| ilomastat | 0.00 | 2640 | 0 |
| metaproterenol | 0.00 | 2641 | 0 |
| AEG3482 | 0.00 | 2642 | 0 |
| tianeptine | 0.00 | 2643 | 0 |
| vinbumine | 0.00 | 2644 | 0 |
| AC-264613 | 0.00 | 2645 | 0 |
| DMeOB | 0.00 | 2646 | 0 |
| bergapten | 0.00 | 2647 | 0 |
| o-3M3FBS | 0.00 | 2648 | 0 |
| somatostatin | 0.00 | 2649 | 0 |
| miglitol | 0.00 | 2650 | 0 |
| DMP-777 | 0.00 | 2651 | 0 |
| thiodiglycol | 0.00 | 2652 | 0 |
| 4-methylgenistein | 0.00 | 2653 | 0 |
| tenatoprazole | 0.00 | 2654 | 0 |
| halofuginone | 0.00 | 2655 | 0 |
| carfilzomib | 0.00 | 2656 | 0 |
| torcitabine | 0.00 | 2657 | 0 |
| hematoxylin | 0.00 | 2658 | 0 |
| cefetamet | 0.00 | 2659 | 0 |
| oxymetazoline | 0.00 | 2660 | 0 |
| SSR128129E | 0.00 | 2661 | 0 |
| promazine | 0.00 | 2662 | 0 |
| exherin | 0.00 | 2663 | 0 |
| CGP-37849 | 0.00 | 2664 | 0 |
| fosfomycin | 0.00 | 2665 | 0 |
| lacosamide | 0.00 | 2666 | 0 |
| AZD6482 | 0.00 | 2667 | 0 |
| hydrocortisone-butyrate | 0.00 | 2668 | 0 |
| dimethisoquin | 0.00 | 2669 | 0 |
| denatonium-benzoate | 0.00 | 2670 | 0 |
| PK-11195 | 0.00 | 2671 | 0 |
| pasiniazid | 0.00 | 2672 | 0 |
| atenolol-(+/−) | 0.00 | 2673 | 0 |
| blebbistatin-(+/−) | 0.00 | 2674 | 0 |
| mesalazine | 0.00 | 2675 | 0 |
| isopropyl-myristate | 0.00 | 2676 | 0 |
| resveratrol | 0.00 | 2677 | 0 |
| ipratropium | 0.00 | 2678 | 0 |
| telatinib | 0.00 | 2679 | 0 |
| ACPC | 0.00 | 2680 | 0 |
| gonadorelin | 0.00 | 2681 | 0 |
| potassium-p-aminobenzoate | 0.00 | 2682 | 0 |
| sophocarpine | 0.00 | 2683 | 0 |
| edrophonium | 0.00 | 2684 | 0 |
| MDL-11939 | 0.00 | 2685 | 0 |
| oxfendazole | 0.00 | 2686 | 0 |
| SKF-83566 | 0.00 | 2687 | 0 |
| lufenuron | 0.00 | 2688 | 0 |
| phenylbenzimidazole-sulfonic-acid | 0.00 | 2689 | 0 |
| propranolol | 0.00 | 2690 | 0 |
| GR-113808 | 0.00 | 2691 | 0 |
| ML161 | 0.00 | 2692 | 0 |
| anagrelide | 0.00 | 2693 | 0 |
| palmitoylcamitine | 0.00 | 2694 | 0 |
| allopurinol | 0.00 | 2695 | 0 |
| ganciclovir | 0.00 | 2696 | 0 |
| tryptophan | 0.00 | 2697 | 0 |
| alfadolone-acetate | 0.00 | 2698 | 0 |
| 2,4-dinitrophenol | 0.00 | 2699 | 0 |
| adatanserin | 0.00 | 2700 | 0 |
| gabapentin | 0.00 | 2701 | 0 |
| PSI-7976 | 0.00 | 2702 | 0 |
| DUP-697 | 0.00 | 2703 | 0 |
| meprylcaine | 0.00 | 2704 | 0 |
| mephenytoin | 0.00 | 2705 | 0 |
| alosetron | 0.00 | 2706 | 0 |
| prulifloxacin | 0.00 | 2707 | 0 |
| 4,5,6,7-tetrabromobenzotriazole | 0.00 | 2708 | 0 |
| PF-06463922 | 0.00 | 2709 | 0 |
| pipecuronium | 0.00 | 2710 | 0 |
| difenpiramide | 0.00 | 2711 | 0 |
| practolol | 0.00 | 2712 | 0 |
| vemurafenib | 0.00 | 2713 | 0 |
| ribociclib | 0.00 | 2714 | 0 |
| brinzolamide | 0.00 | 2715 | 0 |
| VU0364770 | 0.00 | 2716 | 0 |
| isodibut | 0.00 | 2717 | 0 |
| ofloxacin | 0.00 | 2718 | 0 |
| cyamemazine | 0.00 | 2719 | 0 |
| firocoxib | 0.00 | 2720 | 0 |
| bifonazole | 0.00 | 2721 | 0 |
| methapyrilene | 0.00 | 2722 | 0 |
| 3-indolebutyric-acid | 0.00 | 2723 | 0 |
| SEW-2871 | 0.00 | 2724 | 0 |
| GW-842166 | 0.00 | 2725 | 0 |
| beclamide | 0.00 | 2726 | 0 |
| L-NAME | 0.00 | 2727 | 0 |
| EBPC | 0.00 | 2728 | 0 |
| K-MAP | 0.00 | 2729 | 0 |
| triclosan | 0.00 | 2730 | 0 |
| CGP-7930 | 0.00 | 2731 | 0 |
| eltoprazine | 0.00 | 2732 | 0 |
| dexfenfluramine | 0.00 | 2733 | 0 |
| WYE-125132 | 0.00 | 2734 | 0 |
| NA | 0.00 | 2735 | 0 |
| apremilast | 0.00 | 2736 | 0 |
| NAN-190 | 0.00 | 2737 | 0 |
| fenclonine-(+/−) | 0.00 | 2738 | 0 |
| valdecoxib | 0.00 | 2739 | 0 |
| fasudil | 0.00 | 2740 | 0 |
| miconazole | 0.00 | 2741 | 0 |
| hydrastine-(1r,-9s) | 0.00 | 2742 | 0 |
| sulfinpyrazone | 0.00 | 2743 | 0 |
| methazolamide | 0.00 | 2744 | 0 |
| go-6983 | 0.00 | 2745 | 0 |
| NSC-632839 | 0.00 | 2746 | 0 |
| opicapone | 0.00 | 2747 | 0 |
| leucylleucine-methyl-ester | 0.00 | 2748 | 0 |
| INCB-003284 | 0.00 | 2749 | 0 |
| midodrine | 0.00 | 2750 | 0 |
| aptiganel | 0.00 | 2751 | 0 |
| neo-gilurytmal | 0.00 | 2752 | 0 |
| aceclidine | 0.00 | 2753 | 0 |
| oltipraz | 0.00 | 2754 | 0 |
| alfuzosin | 0.00 | 2755 | 0 |
| itacitinib | 0.00 | 2756 | 0 |
| dirithromycin | 0.00 | 2757 | 0 |
| tenidap | 0.00 | 2758 | 0 |
| GR125487 | 0.00 | 2759 | 0 |
| troventol | 0.00 | 2760 | 0 |
| 3-deazaneplanocin-A | 0.00 | 2761 | 0 |
| phthalylsulfacetamide | 0.00 | 2762 | 0 |
| mxolitinib-(S) | 0.00 | 2763 | 0 |
| nolatrexed | 0.00 | 2764 | 0 |
| pentacosanoic-acid | 0.00 | 2765 | 0 |
| uridine | 0.00 | 2766 | 0 |
| D-delta-Tocopherol | 0.00 | 2767 | 0 |
| norethindrone | 0.00 | 2768 | 0 |
| talniflumate | 0.00 | 2769 | 0 |
| faropenem-medoxomil | 0.00 | 2770 | 0 |
| bumetanide | 0.00 | 2771 | 0 |
| pinacidil | 0.00 | 2772 | 0 |
| MK-0773 | 0.00 | 2773 | 0 |
| isaxonine | 0.00 | 2774 | 0 |
| R406 | 0.00 | 2775 | 0 |
| TG-02 | 0.00 | 2776 | 0 |
| 4-aminohippuric-acid | 0.00 | 2777 | 0 |
| iguratimod | 0.00 | 2778 | 0 |
| phlorizin | 0.00 | 2779 | 0 |
| propantheline | 0.00 | 2780 | 0 |
| desoxypeganine | 0.00 | 2781 | 0 |
| talinolol | 0.00 | 2782 | 0 |
| AVL-292 | 0.00 | 2783 | 0 |
| finafloxacin | 0.00 | 2784 | 0 |
| titanocene-dichloride | 0.00 | 2785 | 0 |
| ripasudil | 0.00 | 2786 | 0 |
| alpha-linolenic-acid | 0.00 | 2787 | 0 |
| cyclopentolate | 0.00 | 2788 | 0 |
| mirtazapine | 0.00 | 2789 | 0 |
| vasopressin | 0.00 | 2790 | 0 |
| ARRY-334543 | 0.00 | 2791 | 0 |
| zolpidem | 0.00 | 2792 | 0 |
| estramustine-phosphate | 0.00 | 2793 | 0 |
| garenoxacin | 0.00 | 2794 | 0 |
| tremorine | 0.00 | 2795 | 0 |
| ICI-162846 | 0.00 | 2796 | 0 |
| maribavir | 0.00 | 2797 | 0 |
| fexaramine | 0.00 | 2798 | 0 |
| imiloxan | 0.00 | 2799 | 0 |
| peficitinib | 0.00 | 2800 | 0 |
| suvorexant | 0.00 | 2801 | 0 |
| RS-79948 | 0.00 | 2802 | 0 |
| bicyclol | 0.00 | 2803 | 0 |
| pirodavir | 0.00 | 2804 | 0 |
| equilin | 0.00 | 2805 | 0 |
| ephedrine-(racemic) | 0.00 | 2806 | 0 |
| epothilone-b | 0.00 | 2807 | 0 |
| diclofenac | 0.00 | 2808 | 0 |
| sodium-salicylate | 0.00 | 2809 | 0 |
| etofylline | 0.00 | 2810 | 0 |
| canertinib | 0.00 | 2811 | 0 |
| prednisone | 0.00 | 2812 | 0 |
| avanafil | 0.00 | 2813 | 0 |
| linalool | 0.00 | 2814 | 0 |
| trientine | 0.00 | 2815 | 0 |
| emetine | 0.00 | 2816 | 0 |
| meluadrine | 0.00 | 2817 | 0 |
| E-64 | 0.00 | 2818 | 0 |
| AC-7954-(+/−) | 0.00 | 2819 | 0 |
| oseltamivir-phosphate | 0.00 | 2820 | 0 |
| amifampridine | 0.00 | 2821 | 0 |
| TG-100572 | 0.00 | 2822 | 0 |
| lupanine | 0.00 | 2823 | 0 |
| aklomide | 0.00 | 2824 | 0 |
| CL-218872 | 0.00 | 2825 | 0 |
| gestrinone | 0.00 | 2826 | 0 |
| esculin | 0.00 | 2827 | 0 |
| gefitinib | 0.00 | 2828 | 0 |
| mepazine | 0.00 | 2829 | 0 |
| fanetizole | 0.00 | 2830 | 0 |
| 5-aminolevulinic-acid | 0.00 | 2831 | 0 |
| iloperidone | 0.00 | 2832 | 0 |
| HQK-1001 | 0.00 | 2833 | 0 |
| phenylbutazone | 0.00 | 2834 | 0 |
| picrotoxinin | 0.00 | 2835 | 0 |
| tolbutamide | 0.00 | 2836 | 0 |
| geniposidic-acid | 0.00 | 2837 | 0 |
| atropine | 0.00 | 2838 | 0 |
| citalopram | 0.00 | 2839 | 0 |
| flutrimazole | 0.00 | 2840 | 0 |
| RS-16566 | 0.00 | 2841 | 0 |
| relcovaptan | 0.00 | 2842 | 0 |
| levofloxacin | 0.00 | 2843 | 0 |
| baicalein | 0.00 | 2844 | 0 |
| GW-9662 | 0.00 | 2845 | 0 |
| cyclopiazonic-acid | 0.00 | 2846 | 0 |
| bilobalide | 0.00 | 2847 | 0 |
| perchlozone | 0.00 | 2848 | 0 |
| elvitegravir | 0.00 | 2849 | 0 |
| dexamethasone | 0.00 | 2850 | 0 |
| hexylresorcinol | 0.00 | 2851 | 0 |
| picartamide | 0.00 | 2852 | 0 |
| dropropizine | 0.00 | 2853 | 0 |
| astemizole | 0.00 | 2854 | 0 |
| sumanirole | 0.00 | 2855 | 0 |
| nafamostat | 0.00 | 2856 | 0 |
| tamoxifen | 0.00 | 2857 | 0 |
| xanthone | 0.00 | 2858 | 0 |
| lomerizine | 0.00 | 2859 | 0 |
| hydrocortisone-phosphate | 0.00 | 2860 | 0 |
| maraviroc | 0.00 | 2861 | 0 |
| RG4733 | 0.00 | 2862 | 0 |
| brimonidine | 0.00 | 2863 | 0 |
| BMY-14802 | 0.00 | 2864 | 0 |
| diphemanil | 0.00 | 2865 | 0 |
| sofosbuvir | 0.00 | 2866 | 0 |
| cefmenoxime | 0.00 | 2867 | 0 |
| telmisartan | 0.00 | 2868 | 0 |
| pilaralisib | 0.00 | 2869 | 0 |
| alimemazine | 0.00 | 2870 | 0 |
| ethacizin | 0.00 | 2871 | 0 |
| tazobactam | 0.00 | 2872 | 0 |
| oxypurinol | 0.00 | 2873 | 0 |
| tazarotene | 0.00 | 2874 | 0 |
| ginsenoside-rd | 0.00 | 2875 | 0 |
| phenylbutazone | 0.00 | 2876 | 0 |
| loperamide | 0.00 | 2877 | 0 |
| Z160 | 0.00 | 2878 | 0 |
| acifran | 0.00 | 2879 | 0 |
| moclobemide | 0.00 | 2880 | 0 |
| hydroquinidine | 0.00 | 2881 | 0 |
| mexiletine | 0.00 | 2882 | 0 |
| dexpanthenol | 0.00 | 2883 | 0 |
| cafestol | 0.00 | 2884 | 0 |
| ifosfamide | 0.00 | 2885 | 0 |
| PD-128907 | 0.00 | 2886 | 0 |
| trans-2-Undecenoic-acid | 0.00 | 2887 | 0 |
| BL-5583 | 0.00 | 2888 | 0 |
| LY294002 | 0.00 | 2889 | 0 |
| remimazolam | 0.00 | 2890 | 0 |
| cefoselis | 0.00 | 2891 | 0 |
| ramelteon | 0.00 | 2892 | 0 |
| RHC-80267 | 0.00 | 2893 | 0 |
| amygdalin | 0.00 | 2894 | 0 |
| ammonium-glycyrrhizinate | 0.00 | 2895 | 0 |
| GTP-14564 | 0.00 | 2896 | 0 |
| SB-221284 | 0.00 | 2897 | 0 |
| stanozolol | 0.00 | 2898 | 0 |
| teriflunomide | 0.00 | 2899 | 0 |
| tirofiban | 0.00 | 2900 | 0 |
| phloroglucin | 0.00 | 2901 | 0 |
| PNU-282987 | 0.00 | 2902 | 0 |
| carprofen | 0.00 | 2903 | 0 |
| picrotin | 0.00 | 2904 | 0 |
| sulfathiazole | 0.00 | 2905 | 0 |
| valproic-acid | 0.00 | 2906 | 0 |
| zacopride | 0.00 | 2907 | 0 |
| stemregenin-1 | 0.00 | 2908 | 0 |
| fipronil | 0.00 | 2909 | 0 |
| CNQX | 0.00 | 2910 | 0 |
| isavuconazole | 0.00 | 2911 | 0 |
| bornyl-acetate | 0.00 | 2912 | 0 |
| furazolidone | 0.00 | 2913 | 0 |
| UCL-2077 | 0.00 | 2914 | 0 |
| BAN-ORL-24 | 0.00 | 2915 | 0 |
| ABT-639 | 0.00 | 2916 | 0 |
| diltiazem | 0.00 | 2917 | 0 |
| raloxifene | 0.00 | 2918 | 0 |
| osimertinib | 0.00 | 2919 | 0 |
| dabigatran-etexilate | 0.00 | 2920 | 0 |
| felbinac | 0.00 | 2921 | 0 |
| clebopride | 0.00 | 2922 | 0 |
| piperacetazine | 0.00 | 2923 | 0 |
| dapiprazole | 0.00 | 2924 | 0 |
| glyburide | 0.00 | 2925 | 0 |
| gimeracil | 0.00 | 2926 | 0 |
| budipine | 0.00 | 2927 | 0 |
| buclizine | 0.00 | 2928 | 0 |
| AMPA-(S) | 0.00 | 2929 | 0 |
| valrocemide | 0.00 | 2930 | 0 |
| droperidol | 0.00 | 2931 | 0 |
| lisadimate | 0.00 | 2932 | 0 |
| S26948 | 0.00 | 2933 | 0 |
| tyrphostin-AG-879 | 0.00 | 2934 | 0 |
| tafenoquine | 0.00 | 2935 | 0 |
| taltirelin | 0.00 | 2936 | 0 |
| benfotiamine | 0.00 | 2937 | 0 |
| granisetron | 0.00 | 2938 | 0 |
| alprenolol | 0.00 | 2939 | 0 |
| decamethonium | 0.00 | 2940 | 0 |
| acepromazine | 0.00 | 2941 | 0 |
| sulfamethoxazole | 0.00 | 2942 | 0 |
| epalrestat | 0.00 | 2943 | 0 |
| metirosine | 0.00 | 2944 | 0 |
| butyl-paraben | 0.00 | 2945 | 0 |
| zosuquidar | 0.00 | 2946 | 0 |
| ibutamoren | 0.00 | 2947 | 0 |
| flurothyl | 0.00 | 2948 | 0 |
| perphenazine | 0.00 | 2949 | 0 |
| roflumilast | 0.00 | 2950 | 0 |
| secnidazole | 0.00 | 2951 | 0 |
| verinurad | 0.00 | 2952 | 0 |
| SDZ-220-581 | 0.00 | 2953 | 0 |
| UPF-1069 | 0.00 | 2954 | 0 |
| CG-400549 | 0.00 | 2955 | 0 |
| endo-IWR-1 | 0.00 | 2956 | 0 |
| ETC-1002 | 0.00 | 2957 | 0 |
| misonidazole | 0.00 | 2958 | 0 |
| suxibuzone | 0.00 | 2959 | 0 |
| clofedanol | 0.00 | 2960 | 0 |
| JNJ-1661010 | 0.00 | 2961 | 0 |
| afobazole | 0.00 | 2962 | 0 |
| amitifadine | 0.00 | 2963 | 0 |
| CGS-20625 | 0.00 | 2964 | 0 |
| GR46611 | 0.00 | 2965 | 0 |
| carebastine | 0.00 | 2966 | 0 |
| broxyquinoline | 0.00 | 2967 | 0 |
| ketotifen | 0.00 | 2968 | 0 |
| ganglioside-gm1 | 0.00 | 2969 | 0 |
| methylphenidate | 0.00 | 2970 | 0 |
| epirizole | 0.00 | 2971 | 0 |
| aceneuramic-acid | 0.00 | 2972 | 0 |
| etamsylate | 0.00 | 2973 | 0 |
| muscimol | 0.00 | 2974 | 0 |
| CD-1530 | 0.00 | 2975 | 0 |
| MY-5445 | 0.00 | 2976 | 0 |
| PF-03049423 | 0.00 | 2977 | 0 |
| KU-60019 | 0.00 | 2978 | 0 |
| equol | 0.00 | 2979 | 0 |
| fluocinolone-acetonide | 0.00 | 2980 | 0 |
| tipiracil | 0.00 | 2981 | 0 |
| protirelin | 0.00 | 2982 | 0 |
| melevodopa | 0.00 | 2983 | 0 |
| etoposide-phosphate | 0.00 | 2984 | 0 |
| amantadine | 0.00 | 2985 | 0 |
| 8-M-PDOT | 0.00 | 2986 | 0 |
| SB-206553 | 0.00 | 2987 | 0 |
| adrafinil | 0.00 | 2988 | 0 |
| hypoxanthine | 0.00 | 2989 | 0 |
| loreclezole | 0.00 | 2990 | 0 |
| atipamezole | 0.00 | 2991 | 0 |
| naftopidil | 0.00 | 2992 | 0 |
| enoxacin | 0.00 | 2993 | 0 |
| naloxone | 0.00 | 2994 | 0 |
| eprodisate | 0.00 | 2995 | 0 |
| bergenin | 0.00 | 2996 | 0 |
| GR-135531 | 0.00 | 2997 | 0 |
| rofecoxib | 0.00 | 2998 | 0 |
| TWS-119 | 0.00 | 2999 | 0 |
| nicorandil | 0.00 | 3000 | 0 |
| bicalutamide | 0.00 | 3001 | 0 |
| YM-201636 | 0.00 | 3002 | 0 |
| hydroxyamphetamine | 0.00 | 3003 | 0 |
| ZM-226600 | 0.00 | 3004 | 0 |
| propranolol-(R) | 0.00 | 3005 | 0 |
| LY3023414 | 0.00 | 3006 | 0 |
| rosmarinic-acid | 0.00 | 3007 | 0 |
| quinestrol | 0.00 | 3008 | 0 |
| serotonin | 0.00 | 3009 | 0 |
| carbamazepine | 0.00 | 3010 | 0 |
| benzocaine | 0.00 | 3011 | 0 |
| radafaxine | 0.00 | 3012 | 0 |
| ethoxzolamide | 0.00 | 3013 | 0 |
| anethole-trithione | 0.00 | 3014 | 0 |
| chlortetracycline | 0.00 | 3015 | 0 |
| ftorafur | 0.00 | 3016 | 0 |
| LB-100 | 0.00 | 3017 | 0 |
| abiraterone | 0.00 | 3018 | 0 |
| nafcillin | 0.00 | 3019 | 0 |
| pyrantel | 0.00 | 3020 | 0 |
| BD-1047 | 0.00 | 3021 | 0 |
| cilengitide | 0.00 | 3022 | 0 |
| LE-135 | 0.00 | 3023 | 0 |
| goserelin-acetate | 0.00 | 3024 | 0 |
| paracetamol | 0.00 | 3025 | 0 |
| GW-405833 | 0.00 | 3026 | 0 |
| clorsulon | 0.00 | 3027 | 0 |
| GW-0742 | 0.00 | 3028 | 0 |
| alprostadil | 0.00 | 3029 | 0 |
| etilefrine | 0.00 | 3030 | 0 |
| pargyline | 0.00 | 3031 | 0 |
| tamsulosin | 0.00 | 3032 | 0 |
| U-99194 | 0.00 | 3033 | 0 |
| phenprocoumon | 0.00 | 3034 | 0 |
| fudosteine | 0.00 | 3035 | 0 |
| tolvaptan | 0.00 | 3036 | 0 |
| methylprednisolone | 0.00 | 3037 | 0 |
| sulfapyridine | 0.00 | 3038 | 0 |
| sulfachlorpyridazine | 0.00 | 3039 | 0 |
| astragaloside-a | 0.00 | 3040 | 0 |
| chlorpyrifos | 0.00 | 3041 | 0 |
| taselisib | 0.00 | 3042 | 0 |
| PHTPP | 0.00 | 3043 | 0 |
| meclofenoxate | 0.00 | 3044 | 0 |
| LGX818 | 0.00 | 3045 | 0 |
| piperine | 0.00 | 3046 | 0 |
| balsalazide | 0.00 | 3047 | 0 |
| JDTic | 0.00 | 3048 | 0 |
| quinpirol-(−) | 0.00 | 3049 | 0 |
| etofylline-clofibrate | 0.00 | 3050 | 0 |
| dinoprost | 0.00 | 3051 | 0 |
| doramapimod | 0.00 | 3052 | 0 |
| gabapentin-enacarbil | 0.00 | 3053 | 0 |
| DPCPX | 0.00 | 3054 | 0 |
| betaxolol | 0.00 | 3055 | 0 |
| brimonidine | 0.00 | 3056 | 0 |
| hydroxytoluic-acid | 0.00 | 3057 | 0 |
| WYE-354 | 0.00 | 3058 | 0 |
| piketoprofen | 0.00 | 3059 | 0 |
| evatanepag | 0.00 | 3060 | 0 |
| gemfibrozil | 0.00 | 3061 | 0 |
| amtolmetin-guacil | 0.00 | 3062 | 0 |
| fipexide | 0.00 | 3063 | 0 |
| BIX-02188 | 0.00 | 3064 | 0 |
| methylprednisolone-aceponate | 0.00 | 3065 | 0 |
| ABT-202 | 0.00 | 3066 | 0 |
| ipriflavone | 0.00 | 3067 | 0 |
| trazodone | 0.00 | 3068 | 0 |
| OCO-1112 | 0.00 | 3069 | 0 |
| sodium-tanshinone-ii-a-sulfonate | 0.00 | 3070 | 0 |
| drospirenone | 0.00 | 3071 | 0 |
| sulbutiamine | 0.00 | 3072 | 0 |
| phenyl-aminosalicylate | 0.00 | 3073 | 0 |
| JNJ-37822681 | 0.00 | 3074 | 0 |
| acrivastine | 0.00 | 3075 | 0 |
| dextromethorphan | 0.00 | 3076 | 0 |
| CFTRinh-172 | 0.00 | 3077 | 0 |
| PHCCC | 0.00 | 3078 | 0 |
| phenylpiracetam | 0.00 | 3079 | 0 |
| PF-3274167 | 0.00 | 3080 | 0 |
| deferoxamine-mesylate | 0.00 | 3081 | 0 |
| ftorafur | 0.00 | 3082 | 0 |
| flumecinol | 0.00 | 3083 | 0 |
| flumethasone-pivalate | 0.00 | 3084 | 0 |
| DU-728 | 0.00 | 3085 | 0 |
| chlorphenamine | 0.00 | 3086 | 0 |
| trilostane | 0.00 | 3087 | 0 |
| halothane | 0.00 | 3088 | 0 |
| BRL-50481 | 0.00 | 3089 | 0 |
| MF-101 | 0.00 | 3090 | 0 |
| tarafenacin | 0.00 | 3091 | 0 |
| formoterol | 0.00 | 3092 | 0 |
| chenodeoxycholic-acid | 0.00 | 3093 | 0 |
| levamisole | 0.00 | 3094 | 0 |
| fosaprepitant-dimeglumine | 0.00 | 3095 | 0 |
| cinchocaine | 0.00 | 3096 | 0 |
| capreomycin | 0.00 | 3097 | 0 |
| 1,12-Besm | 0.00 | 3098 | 0 |
| dipivefrine | 0.00 | 3099 | 0 |
| 2-PMDQ | 0.00 | 3100 | 0 |
| SB-328437 | 0.00 | 3101 | 0 |
| niflumic-acid | 0.00 | 3102 | 0 |
| pramiracetam | 0.00 | 3103 | 0 |
| isoxsuprine | 0.00 | 3104 | 0 |
| olvanil | 0.00 | 3105 | 0 |
| lercanidipine | 0.00 | 3106 | 0 |
| busulfan | 0.00 | 3107 | 0 |
| cetrorelix | 0.00 | 3108 | 0 |
| examorelin | 0.00 | 3109 | 0 |
| cipemastat | 0.00 | 3110 | 0 |
| piconol | 0.00 | 3111 | 0 |
| betamipron | 0.00 | 3112 | 0 |
| oxotremorine-m | 0.00 | 3113 | 0 |
| FPL-55712 | 0.00 | 3114 | 0 |
| clotrimazole | 0.00 | 3115 | 0 |
| cabergoline | 0.00 | 3116 | 0 |
| CGP-37157 | 0.00 | 3117 | 0 |
| fananserin | 0.00 | 3118 | 0 |
| pinaverium | 0.00 | 3119 | 0 |
| balapiravir | 0.00 | 3120 | 0 |
| 1-(1,2-Diphenylethyl)piperidine-(+/−) | 0.00 | 3121 | 0 |
| prucalopride | 0.00 | 3122 | 0 |
| macitentan | 0.00 | 3123 | 0 |
| ONX-0914 | 0.00 | 3124 | 0 |
| etomoxir | 0.00 | 3125 | 0 |
| SGC-CBP30 | 0.00 | 3126 | 0 |
| baclofen-(R) | 0.00 | 3127 | 0 |
| huperzine-a | 0.00 | 3128 | 0 |
| nicergoline | 0.00 | 3129 | 0 |
| D-alpha-tocopheryl-succinate | 0.00 | 3130 | 0 |
| lomeguatrib | 0.00 | 3131 | 0 |
| tavaborole | 0.00 | 3132 | 0 |
| rifamycin-sv | 0.00 | 3133 | 0 |
| 2-phenylmelatonin | 0.00 | 3134 | 0 |
| allylthiourea | 0.00 | 3135 | 0 |
| HTMT | 0.00 | 3136 | 0 |
| propoxur | 0.00 | 3137 | 0 |
| chloroxine | 0.00 | 3138 | 0 |
| paromomycin | 0.00 | 3139 | 0 |
| efaroxan | 0.00 | 3140 | 0 |
| preladenant | 0.00 | 3141 | 0 |
| metoprolol | 0.00 | 3142 | 0 |
| cloxyquin | 0.00 | 3143 | 0 |
| methyldopa | 0.00 | 3144 | 0 |
| alfaxalone | 0.00 | 3145 | 0 |
| daidzein | 0.00 | 3146 | 0 |
| dapoxetine | 0.00 | 3147 | 0 |
| purvalanol-b | 0.00 | 3148 | 0 |
| isoleucine | 0.00 | 3149 | 0 |
| AGP-103 | 0.00 | 3150 | 0 |
| zamifenacin | 0.00 | 3151 | 0 |
| bay-w-9798 | 0.00 | 3152 | 0 |
| DAU-5884 | 0.00 | 3153 | 0 |
| gemifloxacin | 0.00 | 3154 | 0 |
| IPAG | 0.00 | 3155 | 0 |
| PRIMA1 | 0.00 | 3156 | 0 |
| m-3M3FBS | 0.00 | 3157 | 0 |
| xamoterol | 0.00 | 3158 | 0 |
| acetylcysteine | 0.00 | 3159 | 0 |
| ALX-5407 | 0.00 | 3160 | 0 |
| SB-657510 | 0.00 | 3161 | 0 |
| hydroxystilbamidine | 0.00 | 3162 | 0 |
| NVP-TAE226 | 0.00 | 3163 | 0 |
| NPY-5RA972 | 0.00 | 3164 | 0 |
| gestodene | 0.00 | 3165 | 0 |
| aspirin | 0.00 | 3166 | 0 |
| pramipexole | 0.00 | 3167 | 0 |
| capsazepine | 0.00 | 3168 | 0 |
| diftalone | 0.00 | 3169 | 0 |
| quinelorane | 0.00 | 3170 | 0 |
| lenalidomide | 0.00 | 3171 | 0 |
| nadifloxacin | 0.00 | 3172 | 0 |
| chloroxylenol | 0.00 | 3173 | 0 |
| asaraldehyde | 0.00 | 3174 | 0 |
| betamethasone-dipropionate | 0.00 | 3175 | 0 |
| nimesulide | 0.00 | 3176 | 0 |
| polidocanol | 0.00 | 3177 | 0 |
| JX-401 | 0.00 | 3178 | 0 |
| potassium-canrenoate | 0.00 | 3179 | 0 |
| ipsapirone | 0.00 | 3180 | 0 |
| dexamethasone-acetate | 0.00 | 3181 | 0 |
| hydrochlorothiazide | 0.00 | 3182 | 0 |
| butorphanol-(+)-tartrate | 0.00 | 3183 | 0 |
| tenoxicam | 0.00 | 3184 | 0 |
| zoxazolamine | 0.00 | 3185 | 0 |
| sarcosine | 0.00 | 3186 | 0 |
| BIBX-1382 | 0.00 | 3187 | 0 |
| 3,3′-diindolylmethane | 0.00 | 3188 | 0 |
| daucosterol | 0.00 | 3189 | 0 |
| octopamine | 0.00 | 3190 | 0 |
| FGIN-1-27 | 0.00 | 3191 | 0 |
| volinanserin | 0.00 | 3192 | 0 |
| SB-258585 | 0.00 | 3193 | 0 |
| adenosine-phosphate | 0.00 | 3194 | 0 |
| cephalomannine | 0.00 | 3195 | 0 |
| canagliflozin | 0.00 | 3196 | 0 |
| isoprenaline | 0.00 | 3197 | 0 |
| CL316243 | 0.00 | 3198 | 0 |
| ethionamide | 0.00 | 3199 | 0 |
| icomucret | 0.00 | 3200 | 0 |
| quetiapine | 0.00 | 3201 | 0 |
| metipranolol | 0.00 | 3202 | 0 |
| nitrofurantoin | 0.00 | 3203 | 0 |
| glutathione | 0.00 | 3204 | 0 |
| idebenone | 0.00 | 3205 | 0 |
| minaprine | 0.00 | 3206 | 0 |
| benfluorex | 0.00 | 3207 | 0 |
| S-Nitrosoglutathione | 0.00 | 3208 | 0 |
| moxonidine | 0.00 | 3209 | 0 |
| cortisone | 0.00 | 3210 | 0 |
| bucladesine | 0.00 | 3211 | 0 |
| proquazone | 0.00 | 3212 | 0 |
| triamcinolone | 0.00 | 3213 | 0 |
| cimetropium | 0.00 | 3214 | 0 |
| AD-5467 | 0.00 | 3215 | 0 |
| metformin | 0.00 | 3216 | 0 |
| acyclovir | 0.00 | 3217 | 0 |
| ampalex | 0.00 | 3218 | 0 |
| sulphadimethoxine | 0.00 | 3219 | 0 |
| KBG | 0.00 | 3220 | 0 |
| 1-octanol | 0.00 | 3221 | 0 |
| aminophylline | 0.00 | 3222 | 0 |
| IKK-2-inhibitor | 0.00 | 3223 | 0 |
| ciprofloxacin | 0.00 | 3224 | 0 |
| hexamethylenebisacetamide | 0.00 | 3225 | 0 |
| 6-chloromelatonin | 0.00 | 3226 | 0 |
| elagolix | 0.00 | 3227 | 0 |
| 1-ethyl-2-pyrrolidone | 0.00 | 3228 | 0 |
| saccharin | 0.00 | 3229 | 0 |
| PF-04217903 | 0.00 | 3230 | 0 |
| felodipine | 0.00 | 3231 | 0 |
| pibenzimol | 0.00 | 3232 | 0 |
| amfenac | 0.00 | 3233 | 0 |
| proflavine-hemisulfate | 0.00 | 3234 | 0 |
| testosterone-enanthate | 0.00 | 3235 | 0 |
| mepivacaine | 0.00 | 3236 | 0 |
| barbital | 0.00 | 3237 | 0 |
| metacresol | 0.00 | 3238 | 0 |
| atazanavir | 0.00 | 3239 | 0 |
| PD-407824 | 0.00 | 3240 | 0 |
| ODM-201 | 0.00 | 3241 | 0 |
| dihomo-gamma-linolenic-acid | 0.00 | 3242 | 0 |
| EPZ005687 | 0.00 | 3243 | 0 |
| irbesartan | 0.00 | 3244 | 0 |
| palonosetron | 0.00 | 3245 | 0 |
| pepstatin | 0.00 | 3246 | 0 |
| sulfamerazine | 0.00 | 3247 | 0 |
| ozanimod | 0.00 | 3248 | 0 |
| ZD-7155 | 0.00 | 3249 | 0 |
| glucosamine | 0.00 | 3250 | 0 |
| acetarsol | 0.00 | 3251 | 0 |
| cinnarazine | 0.00 | 3252 | 0 |
| dyphylline | 0.00 | 3253 | 0 |
| lycopene | 0.00 | 3254 | 0 |
| nerbacadol | 0.00 | 3255 | 0 |
| MR-16728 | 0.00 | 3256 | 0 |
| quinolinic-acid | 0.00 | 3257 | 0 |
| SB-756050 | 0.00 | 3258 | 0 |
| pyrazinamide | 0.00 | 3259 | 0 |
| camylofine-chlorhydrate | 0.00 | 3260 | 0 |
| phytosphingosine | 0.00 | 3261 | 0 |
| loxoprofen | 0.00 | 3262 | 0 |
| iododexetimide | 0.00 | 3263 | 0 |
| eletriptan | 0.00 | 3264 | 0 |
| ertugliflozin | 0.00 | 3265 | 0 |
| N6-methyladenosine | 0.00 | 3266 | 0 |
| arecaidine-propargyl-ester | 0.00 | 3267 | 0 |
| AZDI 446 | 0.00 | 3268 | 0 |
| para-toluenesulfonamide | 0.00 | 3269 | 0 |
| L-368899 | 0.00 | 3270 | 0 |
| xylometazoline | 0.00 | 3271 | 0 |
| bekanamycin | 0.00 | 3272 | 0 |
| beta-funaltrexamine | 0.00 | 3273 | 0 |
| firategrast | 0.00 | 3274 | 0 |
| EX-527 | 0.00 | 3275 | 0 |
| stetaderm | 0.00 | 3276 | 0 |
| methenamine | 0.00 | 3277 | 0 |
| 4,4-pentamethylenepiperidine | 0.00 | 3278 | 0 |
| nefazodone | 0.00 | 3279 | 0 |
| diperodon | 0.00 | 3280 | 0 |
| dinitolmide | 0.00 | 3281 | 0 |
| etofenamate | 0.00 | 3282 | 0 |
| clopidogrel | 0.00 | 3283 | 0 |
| BW-723C86 | 0.00 | 3284 | 0 |
| MK-3697 | 0.00 | 3285 | 0 |
| sevelamer | 0.00 | 3286 | 0 |
| enocitab ine | 0.00 | 3287 | 0 |
| FPH1-(BRD-6125) | 0.00 | 3288 | 0 |
| mebhydroline-1,5- | 0.00 | 3289 | 0 |
| naphtalenedisulfonate | | | |
| umeclidinium | 0.00 | 3290 | 0 |
| pipemidic-acid | 0.00 | 3291 | 0 |
| A922500 | 0.00 | 3292 | 0 |
| proglumide | 0.00 | 3293 | 0 |
| EMD-1214063 | 0.00 | 3294 | 0 |
| carvedilol | 0.00 | 3295 | 0 |
| NBI-27914 | 0.00 | 3296 | 0 |
| broxaterol | 0.00 | 3297 | 0 |
| SR-2640 | 0.00 | 3298 | 0 |
| kaempferol | 0.00 | 3299 | 0 |
| apigenin | 0.00 | 3300 | 0 |
| enzalutamide | 0.00 | 3301 | 0 |
| AZD3463 | 0.00 | 3302 | 0 |
| halopemide | 0.00 | 3303 | 0 |
| rilmenidine | 0.00 | 3304 | 0 |
| oxiniacic-acid | 0.00 | 3305 | 0 |
| carnitine-(D/L) | 0.00 | 3306 | 0 |
| dimercaptosuccinic-acid | 0.00 | 3307 | 0 |
| eltanolone | 0.00 | 3308 | 0 |
| AZD4547 | 0.00 | 3309 | 0 |
| tridihexethyl | 0.00 | 3310 | 0 |
| MK-6096 | 0.00 | 3311 | 0 |
| bavisant | 0.00 | 3312 | 0 |
| procyclidine | 0.00 | 3313 | 0 |
| LY2811376 | 0.00 | 3314 | 0 |
| FR-122047 | 0.00 | 3315 | 0 |
| milnacipran | 0.00 | 3316 | 0 |
| trolox | 0.00 | 3317 | 0 |
| tropesin | 0.00 | 3318 | 0 |
| mexeneone | 0.00 | 3319 | 0 |
| AZD4282 | 0.00 | 3320 | 0 |
| 2-hydroxysaclofen | 0.00 | 3321 | 0 |
| edelfosine | 0.00 | 3322 | 0 |
| tagatose | 0.00 | 3323 | 0 |
| gadoteridol | 0.00 | 3324 | 0 |
| AG-490 | 0.00 | 3325 | 0 |
| salicin | 0.00 | 3326 | 0 |
| LOXO-101 | 0.00 | 3327 | 0 |
| 4-hydroxy-phenazone | 0.00 | 3328 | 0 |
| bendamustine | 0.00 | 3329 | 0 |
| kinetin | 0.00 | 3330 | 0 |
| febuxostat | 0.00 | 3331 | 0 |
| SGX523 | 0.00 | 3332 | 0 |
| isopropamide-iodide | 0.00 | 3333 | 0 |
| eflornithine | 0.00 | 3334 | 0 |
| 1-deoxymannojirimycin | 0.00 | 3335 | 0 |
| CP-91149 | 0.00 | 3336 | 0 |
| carmoxirole | 0.00 | 3337 | 0 |
| PF-04620110 | 0.00 | 3338 | 0 |
| telithromycin | 0.00 | 3339 | 0 |
| penicillin-v-potassium | 0.00 | 3340 | 0 |
| AMG-517 | 0.00 | 3341 | 0 |
| securinine | 0.00 | 3342 | 0 |
| cefminox | 0.00 | 3343 | 0 |
| diphenylpyraline | 0.00 | 3344 | 0 |
| teprenone | 0.00 | 3345 | 0 |
| M-344 | 0.00 | 3346 | 0 |
| azelnidipine | 0.00 | 3347 | 0 |
| almotriptan | 0.00 | 3348 | 0 |
| lobendazole | 0.00 | 3349 | 0 |
| A-7 | 0.00 | 3350 | 0 |
| A-803467 | 0.00 | 3351 | 0 |
| iobenguane | 0.00 | 3352 | 0 |
| AZD2858 | 0.00 | 3353 | 0 |
| nTZDpa | 0.00 | 3354 | 0 |
| CFM-1571 | 0.00 | 3355 | 0 |
| iocetamic-acid | 0.00 | 3356 | 0 |
| ML167 | 0.00 | 3357 | 0 |
| TC2559 | 0.00 | 3358 | 0 |
| FPA-124 | 0.00 | 3359 | 0 |
| ferulic-acid | 0.00 | 3360 | 0 |
| L-Cystine | 0.00 | 3361 | 0 |
| liothyronine | 0.00 | 3362 | 0 |
| melperone | 0.00 | 3363 | 0 |
| adefovir | 0.00 | 3364 | 0 |
| naphazoline | 0.00 | 3365 | 0 |
| manidipine | 0.00 | 3366 | 0 |
| etizolam | 0.00 | 3367 | 0 |
| BMS-587101 | 0.00 | 3368 | 0 |
| mephentermine | 0.00 | 3369 | 0 |
| bucillamine | 0.00 | 3370 | 0 |
| trequinsin | 0.00 | 3371 | 0 |
| cytisine | 0.00 | 3372 | 0 |
| propiverine | 0.00 | 3373 | 0 |
| senicapoc | 0.00 | 3374 | 0 |
| phenazopyridine | 0.00 | 3375 | 0 |
| CGP-55845 | 0.00 | 3376 | 0 |
| streptomycin | 0.00 | 3377 | 0 |
| doxylamine | 0.00 | 3378 | 0 |
| piclamilast | 0.00 | 3379 | 0 |
| prazosin | 0.00 | 3380 | 0 |
| sulfameter | 0.00 | 3381 | 0 |
| anastrozole | 0.00 | 3382 | 0 |
| piromidic-acid | 0.00 | 3383 | 0 |
| enoximone | 0.00 | 3384 | 0 |
| eticlopride | 0.00 | 3385 | 0 |
| lumacaftor | 0.00 | 3386 | 0 |
| fosamprenavir | 0.00 | 3387 | 0 |
| LY303511 | 0.00 | 3388 | 0 |
| diadenosine-tetraphosphate | 0.00 | 3389 | 0 |
| monostearin | 0.00 | 3390 | 0 |
| sorbitol | 0.00 | 3391 | 0 |
| naringenin | 0.00 | 3392 | 0 |
| diazooxonorleucine | 0.00 | 3393 | 0 |
| temocapril | 0.00 | 3394 | 0 |
| emedastine | 0.00 | 3395 | 0 |
| CBS,-N-Cyclohexyl-2- | 0.00 | 3396 | 0 |
| benzothiazolesulfenamide | | | |
| pexidartinib | 0.00 | 3397 | 0 |
| methacholine | 0.00 | 3398 | 0 |
| rupatadine | 0.00 | 3399 | 0 |
| fomocaine | 0.00 | 3400 | 0 |
| procysteine | 0.00 | 3401 | 0 |
| troxipide | 0.00 | 3402 | 0 |
| CK-636 | 0.00 | 3403 | 0 |
| pirenperone | 0.00 | 3404 | 0 |
| tiapride | 0.00 | 3405 | 0 |
| N-acetylcarnosine | 0.00 | 3406 | 0 |
| nifekalant | 0.00 | 3407 | 0 |
| altanserin | 0.00 | 3408 | 0 |
| bepotastine | 0.00 | 3409 | 0 |
| methoxyphenamine | 0.00 | 3410 | 0 |
| conivaptan | 0.00 | 3411 | 0 |
| indocyanine-green | 0.00 | 3412 | 0 |
| mianserin | 0.00 | 3413 | 0 |
| mosapride | 0.00 | 3414 | 0 |
| fruquintinib | 0.00 | 3415 | 0 |
| UNC0321 | 0.00 | 3416 | 0 |
| selenomethionine | 0.00 | 3417 | 0 |
| E7449 | 0.00 | 3418 | 0 |
| linagliptin | 0.00 | 3419 | 0 |
| CGM097 | 0.00 | 3420 | 0 |
| ethambutol | 0.00 | 3421 | 0 |
| nadide | 0.00 | 3422 | 0 |
| nateglinide | 0.00 | 3423 | 0 |
| naftifine | 0.00 | 3424 | 0 |
| quinine | 0.00 | 3425 | 0 |
| nitrendipine | 0.00 | 3426 | 0 |
| co-102862 | 0.00 | 3427 | 0 |
| GSK163090 | 0.00 | 3428 | 0 |
| mitoflaxone | 0.00 | 3429 | 0 |
| carbendazim | 0.00 | 3430 | 0 |
| UBP-302 | 0.00 | 3431 | 0 |
| tiludronate | 0.00 | 3432 | 0 |
| sulfafurazole | 0.00 | 3433 | 0 |
| cefotetan | 0.00 | 3434 | 0 |
| acarbose | 0.00 | 3435 | 0 |
| tucatinib | 0.00 | 3436 | 0 |
| ethynodiol-diacetate | 0.00 | 3437 | 0 |
| cefoselis | 0.00 | 3438 | 0 |
| C-1 | 0.00 | 3439 | 0 |
| lerisetron | 0.00 | 3440 | 0 |
| naphthoquine-phosphate | 0.00 | 3441 | 0 |
| SNAP | 0.00 | 3442 | 0 |
| guanfacine | 0.00 | 3443 | 0 |
| ephedrine | 0.00 | 3444 | 0 |
| AMD 11070 | 0.00 | 3445 | 0 |
| SEP-227900 | 0.00 | 3446 | 0 |
| S-Isopropylisothiourea | 0.00 | 3447 | 0 |
| ML130 | 0.00 | 3448 | 0 |
| ifenprodil | 0.00 | 3449 | 0 |
| triazolam | 0.00 | 3450 | 0 |
| acecainide | 0.00 | 3451 | 0 |
| uridine | 0.00 | 3452 | 0 |
| tiotropium | 0.00 | 3453 | 0 |
| alpha-methylhistamine- | 0.00 | 3454 | 0 |
| dihydrobromide-(R)-(−) | | | |
| apocynin | 0.00 | 3455 | 0 |
| asunaprevir | 0.00 | 3456 | 0 |
| TAME | 0.00 | 3457 | 0 |
| adrenalone | 0.00 | 3458 | 0 |
| tricaprylin | 0.00 | 3459 | 0 |
| ARC-239 | 0.00 | 3460 | 0 |
| JNJ-42165279 | 0.00 | 3461 | 0 |
| KT-433 | 0.00 | 3462 | 0 |
| ZCL-278 | 0.00 | 3463 | 0 |
| fosinopril | 0.00 | 3464 | 0 |
| prednisolone-sodium-phosphate | 0.00 | 3465 | 0 |
| peramivir | 0.00 | 3466 | 0 |
| LCQ908 | 0.00 | 3467 | 0 |
| oxcarbazepine | 0.00 | 3468 | 0 |
| diroximel-fumarate | 0.00 | 3469 | 0 |
| netupitant | 0.00 | 3470 | 0 |
| quinine-ethyl-carbonate | 0.00 | 3471 | 0 |
| LY320135 | 0.00 | 3472 | 0 |
| alfacalcidol | 0.00 | 3473 | 0 |
| TG-100713 | 0.00 | 3474 | 0 |
| schisandrol-b | 0.00 | 3475 | 0 |
| ethyl-vanillin | 0.00 | 3476 | 0 |
| WB-4101 | 0.00 | 3477 | 0 |
| taurine | 0.00 | 3478 | 0 |
| triprolidine | 0.00 | 3479 | 0 |
| CC-930 | 0.00 | 3480 | 0 |
| remacemide | 0.00 | 3481 | 0 |
| efatutazone | 0.00 | 3482 | 0 |
| fenofibrate | 0.00 | 3483 | 0 |
| indium(iii)-isopropoxide | 0.00 | 3484 | 0 |
| indoramin | 0.00 | 3485 | 0 |
| idalopirdine | 0.00 | 3486 | 0 |
| amiprilose | 0.00 | 3487 | 0 |
| levocabastine | 0.00 | 3488 | 0 |
| tafamidis-meglumine | 0.00 | 3489 | 0 |
| paliperidone | 0.00 | 3490 | 0 |
| NPS-2143 | 0.00 | 3491 | 0 |
| SD-208 | 0.00 | 3492 | 0 |
| xibenolol | 0.00 | 3493 | 0 |
| lucitanib | 0.00 | 3494 | 0 |
| GW-7647 | 0.00 | 3495 | 0 |
| viloxazine | 0.00 | 3496 | 0 |
| edoxaban | 0.00 | 3497 | 0 |
| piribedil | 0.00 | 3498 | 0 |
| zidovudine | 0.00 | 3499 | 0 |
| niridazole | 0.00 | 3500 | 0 |
| indole-3-carbinol | 0.00 | 3501 | 0 |
| ICI-199441 | 0.00 | 3502 | 0 |
| chlorogenic-acid | 0.00 | 3503 | 0 |
| cinchonidine | 0.00 | 3504 | 0 |
| dalargin | 0.00 | 3505 | 0 |
| PHA-793887 | 0.00 | 3506 | 0 |
| zaltoprofen | 0.00 | 3507 | 0 |
| cioxacillin | 0.00 | 3508 | 0 |
| U-54494A | 0.00 | 3509 | 0 |
| 2-methyl-5-hydroxytryptamine | 0.00 | 3510 | 0 |
| luteolin | 0.00 | 3511 | 0 |
| furaltadone | 0.00 | 3512 | 0 |
| CDBA | 0.00 | 3513 | 0 |
| tacalcitol | 0.00 | 3514 | 0 |
| timolol | 0.00 | 3515 | 0 |
| harmane | 0.00 | 3516 | 0 |
| mephenesin | 0.00 | 3517 | 0 |
| dimpylate | 0.00 | 3518 | 0 |
| tandospirone | 0.00 | 3519 | 0 |
| gliquidone | 0.00 | 3520 | 0 |
| eszopiclone | 0.00 | 3521 | 0 |
| GBR-13069 | 0.00 | 3522 | 0 |
| refametinib | 0.00 | 3523 | 0 |
| alagebrium | 0.00 | 3524 | 0 |
| L-Arginine | 0.00 | 3525 | 0 |
| phenazopyridine | 0.00 | 3526 | 0 |
| pterostilbene | 0.00 | 3527 | 0 |
| isosteviol | 0.00 | 3528 | 0 |
| brequinar | 0.00 | 3529 | 0 |
| PPT | 0.00 | 3530 | 0 |
| pranlukast | 0.00 | 3531 | 0 |
| CGS-21680 | 0.00 | 3532 | 0 |
| 2-octyldodecan-1-ol | 0.00 | 3533 | 0 |
| ripazepam | 0.00 | 3534 | 0 |
| IMREG-1 | 0.00 | 3535 | 0 |
| dolasetron | 0.00 | 3536 | 0 |
| sertaconazole | 0.00 | 3537 | 0 |
| bicuculline-(+) | 0.00 | 3538 | 0 |
| BP-554 | 0.00 | 3539 | 0 |
| 5-fluorouracil | −0.01 | 3540 | 0 |
| phenacaine | −0.01 | 3541 | 0 |
| oxyclozanide | −0.01 | 3542 | 0 |
| dimaprit | −0.01 | 3543 | 0 |
| metrizoic-acid | −0.01 | 3544 | |
| RN-1747 | −0.01 | 3545 | 0 |
| cordycepin | −0.01 | 3546 | 0 |
| ethamivan | −0.01 | 3547 | 0 |
| brexpiprazole | −0.01 | 3548 | 0 |
| delivert | −0.01 | 3549 | 0 |
| BS-181 | −0.01 | 3550 | 0 |
| puromycin | −0.01 | 3551 | 0 |
| BIX-02189 | −0.01 | 3552 | 0 |
| acemetacin | −0.01 | 3553 | 0 |
| dimercaprol | −0.01 | 3554 | 0 |
| nitrofurazone | −0.01 | 3555 | 0 |
| palifosfamide | −0.01 | 3556 | 0 |
| nizofenone | −0.01 | 3557 | 0 |
| masitinib | −0.01 | 3558 | 0 |
| HA-966-(S)-(-) | −0.01 | 3559 | 0 |
| BIBU-1361 | −0.01 | 3560 | 0 |
| ilepcimide | −0.01 | 3561 | 0 |
| tolcapone | −0.01 | 3562 | 0 |
| sparfloxacin | −0.01 | 3563 | 0 |
| lisuride | −0.01 | 3564 | 0 |
| oxazepam | −0.01 | 3565 | 0 |
| benzoxiquine | −0.01 | 3566 | 0 |
| homoquinolinic-acid | −0.01 | 3567 | 0 |
| A205804 | −0.01 | 3568 | 0 |
| MMPX | −0.01 | 3569 | 0 |
| imidacloprid | −0.01 | 3570 | 0 |
| lixivaptan | −0.01 | 3571 | 0 |
| WAY-200070 | −0.01 | 3572 | 0 |
| guacetisal | −0.01 | 3573 | 0 |
| palmitoylethanolamide | −0.01 | 3574 | 0 |
| 5 -fluoro-3 -pyridyl-methanol | −0.01 | 3575 | 0 |
| PF-4981517 | −0.01 | 3576 | 0 |
| gepefrine | −0.01 | 3577 | 0 |
| RS-0481 | −0.01 | 3578 | 0 |
| octreotide | −0.01 | 3579 | 0 |
| rociletinib | −0.01 | 3580 | 0 |
| detomidine | −0.01 | 3581 | 0 |
| VUF10166 | −0.01 | 3582 | 0 |
| methoxamine | −0.01 | 3583 | 0 |
| butacaine | −0.01 | 3584 | 0 |
| atizoram | −0.01 | 3585 | 0 |
| carzenide | −0.01 | 3586 | 0 |
| BMS-626529 | −0.01 | 3587 | 0 |
| estriol | −0.01 | 3588 | 0 |
| monosod [symbol] luminol | −0.01 | 3589 | 0 |
| norethindrone | −0.01 | 3590 | 0 |
| isoguvacine | −0.01 | 3591 | 0 |
| OC000459 | −0.01 | 3592 | 0 |
| WZ811 | −0.01 | 3593 | 0 |
| azasetron | −0.01 | 3594 | 0 |
| deoxycorticosterone-acetate | −0.01 | 3595 | 0 |
| bucetin | −0.01 | 3596 | 0 |
| mesoridazine | −0.01 | 3597 | 0 |
| valaciclovir | −0.01 | 3598 | 0 |
| chlorphenesin | −0.01 | 3599 | 0 |
| AM-580 | −0.01 | 3600 | 0 |
| apafant | −0.01 | 3601 | 0 |
| NXY-059 | −0.01 | 3602 | 0 |
| alectinib | −0.01 | 3603 | 0 |
| riodipine | −0.01 | 3604 | 0 |
| fenbufen | −0.01 | 3605 | 0 |
| tomelukast | −0.01 | 3606 | 0 |
| T-5224 | −0.01 | 3607 | 0 |
| cefozopran | −0.01 | 3608 | 0 |
| L-152804 | −0.01 | 3609 | 0 |
| acalabrutinib | −0.01 | 3610 | 0 |
| AC-55649 | −0.01 | 3611 | 0 |
| fenigam | −0.01 | 3612 | 0 |
| omeprazole | −0.01 | 3613 | 0 |
| icariin | −0.01 | 3614 | 0 |
| sulindac | −0.01 | 3615 | 0 |
| 1-hexadecanol | −0.01 | 3616 | 0 |
| sulfamethoxypyridazine | −0.01 | 3617 | 0 |
| acetylsalicylsalicylic-acid | −0.01 | 3618 | 0 |
| piperonyl-butoxide | −0.01 | 3619 | 0 |
| probenecid | −0.01 | 3620 | 0 |
| iohexol | −0.01 | 3621 | 0 |
| terconazole | −0.01 | 3622 | 0 |
| ezutromid | −0.01 | 3623 | 0 |
| clindamycin | −0.01 | 3624 | 0 |
| acyclovir | −0.01 | 3625 | 0 |
| synephrine | −0.01 | 3626 | 0 |
| phenolphthalein | −0.01 | 3627 | 0 |
| 12-O-tetradecanoylphorbol-13-acetate | −0.01 | 3628 | 0 |
| nicaraven | −0.01 | 3629 | 0 |
| sparfloxacin | −0.01 | 3630 | 0 |
| amyleine | −0.01 | 3631 | 0 |
| toceranib | −0.01 | 3632 | 0 |
| ampicillin | −0.01 | 3633 | 0 |
| KW-2478 | −0.01 | 3634 | 0 |
| SB-415286 | −0.01 | 3635 | 0 |
| morinidazole | −0.01 | 3636 | 0 |
| acetohydroxamic-acid | −0.01 | 3637 | 0 |
| spermidine | −0.01 | 3638 | 0 |
| SR-57227A | −0.01 | 3639 | 0 |
| cyclobenzaprine | −0.01 | 3640 | 0 |
| DCPIB | −0.01 | 3641 | 0 |
| gamma-linolenic-acid | −0.01 | 3642 | 0 |
| CP-673451 | −0.01 | 3643 | 0 |
| mCPP | −0.01 | 3644 | 0 |
| PD-166793 | −0.01 | 3645 | 0 |
| raltegravir | −0.01 | 3646 | 0 |
| naproxen | −0.01 | 3647 | 0 |
| barnidipine | −0.01 | 3648 | 0 |
| itopride | −0.01 | 3649 | 0 |
| ABT-239 | −0.01 | 3650 | 0 |
| acetanilide | −0.01 | 3651 | 0 |
| PF-04447943 | −0.01 | 3652 | 0 |
| retinaldehyde | −0.01 | 3653 | 0 |
| didanosine | −0.01 | 3654 | 0 |
| actinoquinol | −0.01 | 3655 | 0 |
| F-II440 | −0.01 | 3656 | 0 |
| piracetam | −0.01 | 3657 | 0 |
| roxatidine-acetate | −0.01 | 3658 | 0 |
| SKF-77434 | −0.01 | 3659 | 0 |
| flucloxacillin | −0.01 | 3660 | 0 |
| oxelaidin | −0.01 | 3661 | 0 |
| alizapride | −0.01 | 3662 | 0 |
| clobenpropit | −0.01 | 3663 | 0 |
| chlorocresol | −0.01 | 3664 | 0 |
| esomeprazole | −0.01 | 3665 | 0 |
| abiraterone-acetate | −0.01 | 3666 | 0 |
| rifabutin | −0.01 | 3667 | 0 |
| AMG-487 | −0.01 | 3668 | 0 |
| MK-571 | −0.01 | 3669 | 0 |
| ritodrine | −0.01 | 3670 | 0 |
| neohesperidin-dihydrochalcone | −0.01 | 3671 | 0 |
| acedoben | −0.01 | 3672 | 0 |
| bexarotene | −0.01 | 3673 | 0 |
| MRS3777 | −0.01 | 3674 | 0 |
| dobutamine | −0.01 | 3675 | 0 |
| 7-chlorokynurenic-acid | −0.01 | 3676 | 0 |
| 4-phenolsulfonic-acid | −0.01 | 3677 | 0 |
| eprazinone | −0.01 | 3678 | 0 |
| API-001 | −0.01 | 3679 | 0 |
| rosiglitazone | −0.01 | 3680 | 0 |
| 1-((Z)-3-Chloroallyl)-1,3,5,7- | −0.01 | 3681 | 0 |
| tetraazaadamantan-1-ium | | | |
| carumonam | −0.01 | 3682 | 0 |
| IRL-2500 | −0.01 | 3683 | 0 |
| dipeptamin | −0.01 | 3684 | 0 |
| lorcaserin | −0.01 | 3685 | 0 |
| PD-128907 | −0.01 | 3686 | 0 |
| estrone | −0.01 | 3687 | 0 |
| sasapyrine | −0.01 | 3688 | 0 |
| NVP-ADW742 | −0.01 | 3689 | 0 |
| tolazoline | −0.01 | 3690 | 0 |
| cyromazine | −0.01 | 3691 | 0 |
| erdafitinib | −0.01 | 3692 | 0 |
| aceglutamide | −0.01 | 3693 | 0 |
| articaine | −0.01 | 3694 | 0 |
| noscapine | −0.01 | 3695 | 0 |
| cyacetacide | −0.01 | 3696 | 0 |
| tetrahydrozoline | −0.01 | 3697 | 0 |
| tirapazamine | −0.01 | 3698 | 0 |
| alpelisib | −0.01 | 3699 | 0 |
| glafenine | −0.01 | 3700 | 0 |
| rolipram | −0.01 | 3701 | 0 |
| carboxypyridine-disulfide | −0.01 | 3702 | 0 |
| WZ4003 | −0.01 | 3703 | 0 |
| CHIR-124 | −0.01 | 3704 | 0 |
| mirabegron | −0.01 | 3705 | 0 |
| levothyroxine | −0.01 | 3706 | 0 |
| butaclamol | −0.01 | 3707 | 0 |
| butylsop [symbol] bromide | −0.01 | 3708 | 0 |
| PHT-427 | −0.01 | 3709 | 0 |
| quiflapon | −0.01 | 3710 | 0 |
| gedunin | −0.01 | 3711 | 0 |
| diphenidol | −0.01 | 3712 | 0 |
| mepiroxol | −0.01 | 3713 | 0 |
| calcifediol | −0.01 | 3714 | 0 |
| novobiocin | −0.01 | 3715 | 0 |
| sesamin | −0.01 | 3716 | 0 |
| Ro-10-5824 | −0.01 | 3717 | 0 |
| NNC-55-0396 | −0.01 | 3718 | 0 |
| celiprolol | −0.01 | 3719 | 0 |
| losartan | −0.01 | 3720 | 0 |
| cortisone-acetate | −0.01 | 3721 | 0 |
| chuanxiongzine | −0.01 | 3722 | 0 |
| golgicide-a | −0.01 | 3723 | 0 |
| matrine | −0.01 | 3724 | 0 |
| caffeine | −0.01 | 3725 | 0 |
| DMAB-anabaseine | −0.01 | 3726 | 0 |
| iproniazid | −0.01 | 3727 | 0 |
| BVT-948 | −0.01 | 3728 | 0 |
| SCH-28080 | −0.01 | 3729 | 0 |
| TG-100801 | −0.01 | 3730 | 0 |
| cis-ACPD | −0.01 | 3731 | 0 |
| U-0126 | −0.01 | 3732 | 0 |
| N-alpha-Methylhistamine- | −0.01 | 3733 | 0 |
| dihydrochloride | | | |
| INC-280 | −0.01 | 3734 | 0 |
| trichloroacetic-acid | −0.01 | 3735 | 0 |
| difloxacin | −0.01 | 3736 | 0 |
| aspartame | −0.01 | 3737 | 0 |
| ibuprofen | −0.01 | 3738 | 0 |
| ammonium-lactate | −0.01 | 3739 | 0 |
| TXA127 | −0.01 | 3740 | 0 |
| tylosin | −0.01 | 3741 | 0 |
| laurocapram | −0.01 | 3742 | 0 |
| vardenafil | −0.01 | 3743 | 0 |
| lobenzarit | −0.01 | 3744 | 0 |
| isepamicin | −0.01 | 3745 | 0 |
| DS2-(806622) | −0.01 | 3746 | 0 |
| perampanel | −0.01 | 3747 | 0 |
| pidotimod | −0.01 | 3748 | 0 |
| pirenzepine | −0.01 | 3749 | 0 |
| LDN-212854 | −0.01 | 3750 | 0 |
| brucine | −0.01 | 3751 | 0 |
| sulbactam-pivoxil | −0.01 | 3752 | 0 |
| 2-pyridyl-ethylamine | −0.01 | 3753 | 0 |
| darunavir | −0.01 | 3754 | 0 |
| phenothrin | −0.01 | 3755 | 0 |
| WAY-161503 | −0.01 | 3756 | 0 |
| hyoscyamine | −0.01 | 3757 | 0 |
| entinostat | −0.01 | 3758 | 0 |
| chlorquinaldol | −0.01 | 3759 | 0 |
| pafuramidine | −0.01 | 3760 | 0 |
| levosimendan | −0.01 | 3761 | 0 |
| sulpiride | −0.01 | 3762 | 0 |
| L-365260 | −0.01 | 3763 | 0 |
| lithium-acetoacetate | −0.01 | 3764 | 0 |
| letrozole | −0.01 | 3765 | 0 |
| UNC0224 | −0.01 | 3766 | 0 |
| 3-bromo-7-nitroindazole | −0.01 | 3767 | 0 |
| compound-w | −0.01 | 3768 | 0 |
| veliparib | −0.01 | 3769 | 0 |
| cimaterol | −0.01 | 3770 | 0 |
| dehydroepiandrosterone-sulfate | −0.01 | 3771 | 0 |
| pazufloxacin | −0.01 | 3772 | 0 |
| tasquinimod | −0.01 | 3773 | 0 |
| hordenine | −0.01 | 3774 | 0 |
| benznidazole | −0.01 | 3775 | 0 |
| gallic-acid | −0.01 | 3776 | 0 |
| azalomycin-b | −0.01 | 3777 | 0 |
| meclofenamic-acid | −0.01 | 3778 | 0 |
| dehydroepiandrosterone | −0.01 | 3779 | 0 |
| varespladib | −0.01 | 3780 | 0 |
| PCI-34051 | −0.01 | 3781 | 0 |
| inositol | −0.01 | 3782 | 0 |
| nastorazepide | −0.01 | 3783 | 0 |
| avobenzone | −0.01 | 3784 | 0 |
| mitiglinide | −0.01 | 3785 | 0 |
| S-Sulfo-L-cysteine-sodium-salt | −0.01 | 3786 | 0 |
| cefaclor | −0.01 | 3787 | 0 |
| VX-765 | −0.01 | 3788 | 0 |
| Ro-04-5595 | −0.01 | 3789 | 0 |
| vernakalant | −0.01 | 3790 | 0 |
| isofloxythepin | −0.01 | 3791 | 0 |
| 7-methoxytacrine | −0.01 | 3792 | 0 |
| pirenoxine | −0.01 | 3793 | 0 |
| olmutinib | −0.01 | 3794 | 0 |
| timonacic | −0.01 | 3795 | 0 |
| fosphenytoin | −0.01 | 3796 | 0 |
| 2-ethyl-1,3-hexanediol | −0.01 | 3797 | 0 |
| tyrphostin-AG-1478 | −0.01 | 3798 | 0 |
| icaritin | −0.01 | 3799 | 0 |
| levo-phencynonate | −0.01 | 3800 | 0 |
| irosustat | −0.01 | 3801 | 0 |
| CPI-613 | −0.01 | 3802 | 0 |
| adrenosterone | −0.01 | 3803 | 0 |
| paricalcitol | −0.01 | 3804 | 0 |
| tetramisole | −0.01 | 3805 | 0 |
| spiroxatrine | −0.01 | 3806 | 0 |
| dibenzepine | −0.01 | 3807 | 0 |
| eupatilin | −0.01 | 3808 | 0 |
| levosulpiride | −0.01 | 3809 | 0 |
| deferiprone | −0.01 | 3810 | 0 |
| oxethazaine | −0.01 | 3811 | 0 |
| bromhexine | −0.01 | 3812 | 0 |
| levetiracetam | −0.01 | 3813 | 0 |
| deoxycholic-acid | −0.01 | 3814 | 0 |
| RX-821002 | −0.01 | 3815 | 0 |
| sulfacetamide | −0.01 | 3816 | 0 |
| methoxyamine | −0.01 | 3817 | 0 |
| Y-134 | −0.01 | 3818 | 0 |
| kainic-acid | −0.01 | 3819 | 0 |
| bimatoprost | −0.01 | 3820 | 0 |
| metronidazole | −0.01 | 3821 | 0 |
| QX-222 | −0.01 | 3822 | 0 |
| rolziracetam | −0.01 | 3823 | 0 |
| cladribine | −0.01 | 3824 | 0 |
| clofarabine | −0.01 | 3825 | 0 |
| imiquimod | −0.01 | 3826 | 0 |
| naringin | −0.01 | 3827 | 0 |
| safingol | −0.01 | 3828 | 0 |
| apixaban | −0.01 | 3829 | 0 |
| efavirenz | −0.01 | 3830 | 0 |
| nedocromil | −0.01 | 3831 | 0 |
| 2-deoxyglucose | −0.01 | 3832 | 0 |
| secnidazole | −0.01 | 3833 | 0 |
| PF-03084014 | −0.01 | 3834 | 0 |
| favipiravir | −0.01 | 3835 | 0 |
| I-BZA | −0.01 | 3836 | 0 |
| sitagliptin | −0.01 | 3837 | 0 |
| MM77 | −0.01 | 3838 | 0 |
| CP-724714 | −0.01 | 3839 | 0 |
| lofexidine | −0.01 | 3840 | 0 |
| pentetic-acid | −0.01 | 3841 | 0 |
| nizatidine | −0.01 | 3842 | 0 |
| GSK2816126 | −0.01 | 3843 | 0 |
| hyodeoxycholic-acid | −0.01 | 3844 | 0 |
| diosmetin | −0.01 | 3845 | 0 |
| PP242 | −0.01 | 3846 | 0 |
| nifursol | −0.01 | 3847 | 0 |
| rivaroxaban | −0.01 | 3848 | 0 |
| 3-AQC | −0.01 | 3849 | 0 |
| AZ-10417808 | −0.01 | 3850 | 0 |
| AEE788 | −0.01 | 3851 | 0 |
| cinchophen | −0.01 | 3852 | 0 |
| butylated-hydroxyanisole | −0.01 | 3853 | 0 |
| ibandronate | −0.01 | 3854 | 0 |
| nomifensine | −0.01 | 3855 | 0 |
| XE-991 | −0.01 | 3856 | 0 |
| ditiocarb-sodium-trihydrate | −0.01 | 3857 | 0 |
| omecamtiv-mecarbil | −0.01 | 3858 | 0 |
| sulfamethizole | −0.01 | 3859 | 0 |
| DMH1 | −0.01 | 3860 | 0 |
| VS-4718 | −0.01 | 3861 | 0 |
| zopolrestat | −0.01 | 3862 | 0 |
| 5-BDBD | −0.01 | 3863 | 0 |
| CL-225385 | −0.01 | 3864 | 0 |
| danofloxacin | −0.01 | 3865 | 0 |
| CHIR-99021 | −0.01 | 3866 | 0 |
| C646 | −0.01 | 3867 | 0 |
| imidapril | −0.01 | 3868 | 0 |
| kynurenic-acid | −0.01 | 3869 | 0 |
| oxprenolol | −0.01 | 3870 | 0 |
| methimazole | −0.01 | 3871 | 0 |
| temoporfin | −0.01 | 3872 | 0 |
| laquinimod | −0.01 | 3873 | 0 |
| AZD6765 | −0.01 | 3874 | 0 |
| tiquizium | −0.01 | 3875 | 0 |
| pizotifen | −0.01 | 3876 | 0 |
| ABT-724 | −0.01 | 3877 | 0 |
| schisandrin-b | −0.01 | 3878 | 0 |
| GBR-12935 | −0.01 | 3879 | 0 |
| ulipristal | −0.01 | 3880 | 0 |
| salvinorin-a | −0.01 | 3881 | 0 |
| zardaverine | −0.01 | 3882 | 0 |
| CGP-57380 | −0.01 | 3883 | 0 |
| SC-9 | −0.01 | 3884 | 0 |
| tacrine | −0.01 | 3885 | 0 |
| honokiol | −0.01 | 3886 | 0 |
| balicatib | −0.01 | 3887 | 0 |
| benztropine-mesylate | −0.01 | 3888 | 0 |
| thioctic-acid | −0.01 | 3889 | 0 |
| calcium-gluceptate | −0.01 | 3890 | 0 |
| nalbuphine | −0.01 | 3891 | 0 |
| mestranol | −0.01 | 3892 | 0 |
| palosuran | −0.01 | 3893 | 0 |
| salicylic-acid | −0.01 | 3894 | 0 |
| 5-methylfurmethiodide | −0.01 | 3895 | 0 |
| selegiline | −0.01 | 3896 | 0 |
| piroxicam | −0.01 | 3897 | 0 |
| CI-976 | −0.01 | 3898 | 0 |
| guanidine | −0.01 | 3899 | 0 |
| VP-20629 | −0.01 | 3900 | 0 |
| rufloxacin | −0.01 | 3901 | 0 |
| psoralen | −0.01 | 3902 | 0 |
| stavudine | −0.01 | 3903 | 0 |
| GDC-0879 | −0.01 | 3904 | 0 |
| 5-methylhydantoin-(L) | −0.01 | 3905 | 0 |
| cibenzoline | −0.01 | 3906 | 0 |
| sumatriptan | −0.01 | 3907 | 0 |
| netilmicin | −0.01 | 3908 | 0 |
| tioxolone | −0.01 | 3909 | 0 |
| cediranib | −0.01 | 3910 | 0 |
| indapamide | −0.01 | 3911 | 0 |
| diethyltoluamide | −0.01 | 3912 | 0 |
| carteolol | −0.01 | 3913 | 0 |
| bifendate | −0.01 | 3914 | 0 |
| UNC669 | −0.01 | 3915 | 0 |
| anidulafungin | −0.01 | 3916 | 0 |
| evans-blue | −0.01 | 3917 | 0 |
| fenoverine | −0.01 | 3918 | 0 |
| gatifloxacin | −0.01 | 3919 | 0 |
| mesna | −0.01 | 3920 | 0 |
| navitoclax | −0.01 | 3921 | 0 |
| sarpogrelate | −0.01 | 3922 | 0 |
| AM-281 | −0.01 | 3923 | 0 |
| N-MPPP | −0.01 | 3924 | 0 |
| benzbromarone | −0.01 | 3925 | 0 |
| candesartan | −0.01 | 3926 | 0 |
| 4-chlorophenylguanidine | −0.01 | 3927 | 0 |
| adenosine-triphosphate | −0.01 | 3928 | 0 |
| enilconazole | −0.01 | 3929 | 0 |
| NM107 | −0.01 | 3930 | 0 |
| mofezolac | −0.01 | 3931 | 0 |
| sulfadoxine | −0.01 | 3932 | 0 |
| GTS21 | −0.01 | 3933 | 0 |
| apalutamide | −0.01 | 3934 | 0 |
| oxonic-acid | −0.01 | 3935 | 0 |
| KRN-633 | −0.01 | 3936 | 0 |
| M-14157 | −0.01 | 3937 | 0 |
| usniacin-(+) | −0.01 | 3938 | 0 |
| RS-67506 | −0.01 | 3939 | 0 |
| colforsin | −0.01 | 3940 | 0 |
| PNU-22394 | −0.01 | 3941 | 0 |
| pantoprazole | −0.01 | 3942 | 0 |
| cephapirin | −0.01 | 3943 | 0 |
| castanospermine | −0.01 | 3944 | 0 |
| pramoxine | −0.01 | 3945 | 0 |
| diphencyprone | −0.01 | 3946 | 0 |
| NPPB | −0.01 | 3947 | 0 |
| pyridoxamine | −0.01 | 3948 | 0 |
| mitoxantrone | −0.01 | 3949 | 0 |
| clorprenaline | −0.01 | 3950 | 0 |
| targinine | −0.01 | 3951 | 0 |
| bendazac | −0.01 | 3952 | 0 |
| Ro-28-1675 | −0.01 | 3953 | 0 |
| drotaverine | −0.01 | 3954 | 0 |
| procodazole | −0.01 | 3955 | 0 |
| acetylcysteine | −0.01 | 3956 | 0 |
| L-745870 | −0.01 | 3957 | 0 |
| tozadenant | −0.01 | 3958 | 0 |
| captopril | −0.01 | 3959 | 0 |
| secalciferol | −0.01 | 3960 | 0 |
| AZD7762 | −0.01 | 3961 | 0 |
| CCT018159 | −0.01 | 3962 | 0 |
| sulfametopyrazine | −0.01 | 3963 | 0 |
| U-0126 | −0.01 | 3964 | 0 |
| eluxadoline | −0.01 | 3965 | 0 |
| PSI-6130 | −0.01 | 3966 | 0 |
| SB-200646 | −0.01 | 3967 | 0 |
| ABT-491 | −0.01 | 3968 | 0 |
| clarithromycin | −0.01 | 3969 | 0 |
| ospemifene | −0.01 | 3970 | 0 |
| 5-iodo-A-85380 | −0.01 | 3971 | 0 |
| CTS21166 | −0.01 | 3972 | 0 |
| sulbactam | −0.01 | 3973 | 0 |
| alogliptin | −0.01 | 3974 | 0 |
| SMI-4a | −0.01 | 3975 | 0 |
| pirfenidone | −0.01 | 3976 | 0 |
| protionamide | −0.01 | 3977 | 0 |
| YO-01027 | −0.01 | 3978 | 0 |
| mizoribine | −0.01 | 3979 | 0 |
| adiphenine | −0.01 | 3980 | 0 |
| nandrolone | −0.01 | 3981 | 0 |
| ropivacaine | −0.01 | 3982 | 0 |
| ID-8 | −0.01 | 3983 | 0 |
| phenylbutyrate | −0.01 | 3984 | 0 |
| WAY-213613 | −0.01 | 3985 | 0 |
| semaxanib | −0.01 | 3986 | 0 |
| AM-24 | −0.01 | 3987 | 0 |
| frentizole | −0.01 | 3988 | 0 |
| meticrane | −0.01 | 3989 | 0 |
| clioquinol | −0.01 | 3990 | 0 |
| regadenoson | −0.01 | 3991 | 0 |
| Ro-08-2750 | −0.01 | 3992 | 0 |
| ganaxolone | −0.01 | 3993 | 0 |
| lafutidine | −0.01 | 3994 | 0 |
| HEMADO | −0.01 | 3995 | 0 |
| cefuroxime | −0.01 | 3996 | 0 |
| K02288 | −0.01 | 3997 | 0 |
| ABT-702 | −0.01 | 3998 | 0 |
| ioxilan | −0.01 | 3999 | 0 |
| U-50488-(−) | −0.01 | 4000 | 0 |
| baricitinib | −0.01 | 4001 | 0 |
| tapentadol | −0.01 | 4002 | 0 |
| labetalol | −0.01 | 4003 | 0 |
| tropisetron | −0.01 | 4004 | 0 |
| nimodipine | −0.01 | 4005 | 0 |
| azodicarbonamide | −0.01 | 4006 | 0 |
| indoximod | −0.01 | 4007 | 0 |
| terazosin | −0.01 | 4008 | 0 |
| dofetilide | −0.01 | 4009 | 0 |
| synephrine | −0.01 | 4010 | 0 |
| sacubitril | −0.01 | 4011 | 0 |
| UNC1215 | −0.01 | 4012 | 0 |
| CGP-12177 | −0.01 | 4013 | 0 |
| rostafuroxine | −0.01 | 4014 | 0 |
| Pyrithyldione | −0.01 | 4015 | 0 |
| trioxsalen | −0.01 | 4016 | 0 |
| calcitriol | −0.01 | 4017 | 0 |
| flumequine | −0.01 | 4018 | 0 |
| dalfampridine | −0.01 | 4019 | 0 |
| ajmaline | −0.01 | 4020 | 0 |
| varenicline | −0.01 | 4021 | 0 |
| dasabuvir | −0.01 | 4022 | 0 |
| 1-naphthyl-PP1 | −0.01 | 4023 | 0 |
| prenylamine | −0.01 | 4024 | 0 |
| homosalate | −0.01 | 4025 | 0 |
| salicyl-alcohol | −0.01 | 4026 | 0 |
| faropenem | −0.01 | 4027 | 0 |
| benzylpenicillin | −0.01 | 4028 | 0 |
| ecabapide | −0.01 | 4029 | 0 |
| imexon | −0.01 | 4030 | 0 |
| levobunolol-(+) | −0.01 | 4031 | 0 |
| quipazine | −0.01 | 4032 | 0 |
| L-Quisqualic-acid | −0.01 | 4033 | 0 |
| BD-1008 | −0.01 | 4034 | 0 |
| RS-45041-190 | −0.01 | 4035 | 0 |
| MK-0812 | −0.01 | 4036 | 0 |
| foscamet | −0.01 | 4037 | 0 |
| calcium-levofolinate | −0.01 | 4038 | 0 |
| RS-67333 | −0.01 | 4039 | 0 |
| CDK1-5-inhibitor | −0.01 | 4040 | 0 |
| chlorazanil | −0.01 | 4041 | 0 |
| EIT-hydrobromide | −0.01 | 4042 | 0 |
| MM-102 | −0.01 | 4043 | 0 |
| istaroxime | −0.01 | 4044 | 0 |
| clenbuterol | −0.01 | 4045 | 0 |
| delapril | −0.01 | 4046 | 0 |
| norethisterone-enanthate | −0.01 | 4047 | 0 |
| H-89 | −0.01 | 4048 | 0 |
| ICI-215,001 | −0.01 | 4049 | 0 |
| midafotel | −0.01 | 4050 | 0 |
| isoconazole | −0.01 | 4051 | 0 |
| chlorothiazide | −0.01 | 4052 | 0 |
| marbofloxacin | −0.01 | 4053 | 0 |
| lestaurtinib | −0.01 | 4054 | 0 |
| aminomethyltransferase | −0.01 | 4055 | 0 |
| rebamipide | −0.01 | 4056 | 0 |
| tetrahydrouridine | −0.01 | 4057 | 0 |
| WAY-362450 | −0.01 | 4058 | 0 |
| chlorpromazine | −0.01 | 4059 | 0 |
| meglumine | −0.01 | 4060 | 0 |
| nepicastat | −0.01 | 4061 | 0 |
| N-acetyl-D-glucosamine | −0.01 | 4062 | 0 |
| IEM1754 | −0.01 | 4063 | 0 |
| obidoxime | −0.01 | 4064 | 0 |
| timofibrate | −0.01 | 4065 | 0 |
| fluconazole | −0.01 | 4066 | 0 |
| TGX-221 | −0.01 | 4067 | 0 |
| procaterol | −0.01 | 4068 | 0 |
| TTNPB | −0.01 | 4069 | 0 |
| FPL-64176 | −0.01 | 4070 | 0 |
| salazodine | −0.01 | 4071 | 0 |
| nikethamide | −0.01 | 4072 | 0 |
| norfloxacin | −0.01 | 4073 | 0 |
| erythritol | −0.01 | 4074 | 0 |
| telcagepant | −0.01 | 4075 | 0 |
| sevoflurane | −0.01 | 4076 | 0 |
| octocrylene | −0.01 | 4077 | 0 |
| TAK-285 | −0.01 | 4078 | 0 |
| docosanol | −0.01 | 4079 | 0 |
| tyloxapol | −0.01 | 4080 | 0 |
| atrasentan | −0.01 | 4081 | 0 |
| dimetridazole | −0.01 | 4082 | 0 |
| clomethiazole | −0.01 | 4083 | 0 |
| amoxicillin | −0.01 | 4084 | 0 |
| carglumic-acid | −0.01 | 4085 | 0 |
| ambenonium | −0.01 | 4086 | 0 |
| etoricoxib | −0.01 | 4087 | 0 |
| vanitiolide | −0.01 | 4088 | 0 |
| idasanutlin | −0.01 | 4089 | 0 |
| luliconazole | −0.01 | 4090 | 0 |
| prasterone-acetate | −0.01 | 4091 | 0 |
| daptomycin | −0.01 | 4092 | 0 |
| kakonein | −0.01 | 4093 | 0 |
| L-732,138 | −0.01 | 4094 | 0 |
| AH6809 | −0.01 | 4095 | 0 |
| methoxyflurane | −0.01 | 4096 | 0 |
| spironolactone | −0.01 | 4097 | 0 |
| hydroxyurea | −0.01 | 4098 | 0 |
| ibrutinib | −0.01 | 4099 | 0 |
| GR-79236 | −0.01 | 4100 | 0 |
| SX-011 | −0.01 | 4101 | 0 |
| cinaciguat | −0.01 | 4102 | 0 |
| PNU-120596 | −0.01 | 4103 | 0 |
| thiram | −0.01 | 4104 | 0 |
| oxacillin | −0.01 | 4105 | 0 |
| myricitrin | −0.01 | 4106 | 0 |
| aloe-emodin | −0.01 | 4107 | 0 |
| valganciclovir | −0.01 | 4108 | 0 |
| quinagolide | −0.01 | 4109 | 0 |
| 10-DEBC | −0.01 | 4110 | 0 |
| rhein | −0.01 | 4111 | 0 |
| clorgiline | −0.01 | 4112 | 0 |
| Pentostatin | −0.01 | 4113 | 0 |
| tranexamic-acid | −0.01 | 4114 | 0 |
| CEP-37440 | −0.01 | 4115 | 0 |
| m-Chlorophenylbiguanide | −0.01 | 4116 | 0 |
| beta-amyloid-synthesis-inhibitor | −0.01 | 4117 | 0 |
| etidronic-acid | −0.01 | 4118 | 0 |
| tedizolid-phosphate | −0.01 | 4119 | 0 |
| fenthion | −0.01 | 4120 | 0 |
| YS-035 | −0.01 | 4121 | 0 |
| 3-amino-benzamide | −0.01 | 4122 | 0 |
| JTC-801 | −0.01 | 4123 | 0 |
| sulfanilamide | −0.01 | 4124 | 0 |
| aminolevulinic-acid-benzyl-ester | −0.01 | 4125 | 0 |
| 7-nitroindazole | −0.01 | 4126 | 0 |
| 2-chloro-N6-cyclopentyladenosine | −0.01 | 4127 | 0 |
| pinocembrin | −0.01 | 4128 | 0 |
| FGIN-1-43 | −0.01 | 4129 | 0 |
| mefenamic-acid | −0.01 | 4130 | 0 |
| GABA-linoleamide | −0.01 | 4131 | 0 |
| TRV130 | −0.01 | 4132 | 0 |
| tirasemtiv | −0.01 | 4133 | 0 |
| LY2109761 | −0.01 | 4134 | 0 |
| risperidone | −0.01 | 4135 | 0 |
| paroxetine | −0.01 | 4136 | 0 |
| AMG-837 | −0.01 | 4137 | 0 |
| ibutilide | −0.01 | 4138 | 0 |
| valsartan | −0.01 | 4139 | 0 |
| isamoltane | −0.01 | 4140 | 0 |
| atglistatin | −0.01 | 4141 | 0 |
| pralidoxime-chloride | −0.01 | 4142 | 0 |
| glasdegib | −0.01 | 4143 | 0 |
| niacin | −0.01 | 4144 | 0 |
| phenytoin | −0.01 | 4145 | 0 |
| heptaminol | −0.01 | 4146 | 0 |
| nebracetam | −0.01 | 4147 | 0 |
| sorbic-acid | −0.01 | 4148 | 0 |
| taprenepag | −0.01 | 4149 | 0 |
| urapidil | −0.01 | 4150 | 0 |
| trimetozine | −0.01 | 4151 | 0 |
| moxifloxacin | −0.01 | 4152 | 0 |
| merbromin | −0.01 | 4153 | 0 |
| emodin | −0.01 | 4154 | 0 |
| chlorproguanil | −0.01 | 4155 | 0 |
| pyrazinoylguanidine | −0.01 | 4156 | 0 |
| anabasine | −0.01 | 4157 | 0 |
| chicago-sky-blue-6b | −0.01 | 4158 | 0 |
| nabumetone | −0.01 | 4159 | 0 |
| mdivi-1 | −0.01 | 4160 | 0 |
| marimastat | −0.01 | 4161 | 0 |
| LY225910 | −0.01 | 4162 | 0 |
| metazosin | −0.01 | 4163 | 0 |
| ponesimod | −0.01 | 4164 | 0 |
| quercetin | −0.01 | 4165 | 0 |
| flecainide | −0.01 | 4166 | 0 |
| XL-647 | −0.01 | 4167 | 0 |
| AKBA | −0.01 | 4168 | 0 |
| pozanicline | −0.01 | 4169 | 0 |
| zonisamide | −0.01 | 4170 | 0 |
| cianidanol | −0.01 | 4171 | 0 |
| indomethacin | −0.01 | 4172 | 0 |
| piperine | −0.01 | 4173 | 0 |
| diclofenamide | −0.01 | 4174 | 0 |
| flopropione | −0.01 | 4175 | 0 |
| levomepromazine | −0.01 | 4176 | 0 |
| ADD-233089 | −0.01 | 4177 | 0 |
| N-methyllidocaine-iodide | −0.01 | 4178 | 0 |
| pimavanserin | −0.01 | 4179 | 0 |
| levobunolol-(+/−) | −0.01 | 4180 | 0 |
| GR-144053 | −0.01 | 4181 | 0 |
| phenindione | −0.01 | 4182 | 0 |
| allantoin | −0.01 | 4183 | 0 |
| proxodolol | −0.01 | 4184 | 0 |
| oxaprozin | −0.01 | 4185 | 0 |
| loxoprofen | −0.01 | 4186 | 0 |
| YM-976 | −0.01 | 4187 | 0 |
| JNJ-17203212 | −0.01 | 4188 | 0 |
| talnetant | −0.01 | 4189 | 0 |
| telmesteine | −0.01 | 4190 | 0 |
| acetyl-1-leucine | −0.01 | 4191 | 0 |
| LY2886721 | −0.01 | 4192 | 0 |
| CGP-13501 | −0.01 | 4193 | 0 |
| U-0521 | −0.01 | 4194 | 0 |
| norethindrone-acetate | −0.01 | 4195 | 0 |
| trimetazidine | −0.01 | 4196 | 0 |
| agmatine | −0.01 | 4197 | 0 |
| NNC-711 | −0.01 | 4198 | 0 |
| eplerenone | −0.01 | 4199 | 0 |
| ioxaglic-acid | −0.01 | 4200 | 0 |
| naltrexone | −0.01 | 4201 | 0 |
| rucaparib | −0.01 | 4202 | 0 |
| azelaic-acid | −0.01 | 4203 | 0 |
| landiolol | −0.01 | 4204 | 0 |
| theophylline | −0.01 | 4205 | 0 |
| aminohydroxybutyric-acid | −0.01 | 4206 | 0 |
| posaconazole | −0.01 | 4207 | 0 |
| mefexamide | −0.01 | 4208 | 0 |
| atovaquone | −0.01 | 4209 | 0 |
| ML-9 | −0.01 | 4210 | 0 |
| methylthiouracil | −0.01 | 4211 | 0 |
| KY02111 | −0.01 | 4212 | 0 |
| lovastatin | −0.01 | 4213 | 0 |
| dazmegrel | −0.01 | 4214 | 0 |
| saquinavir | −0.01 | 4215 | 0 |
| ebselen | −0.01 | 4216 | 0 |
| vesamicol | −0.01 | 4217 | 0 |
| ciglitazone | −0.01 | 4218 | 0 |
| nutlin-3 | −0.01 | 4219 | 0 |
| tiotidine | −0.01 | 4220 | 0 |
| monastrol | −0.01 | 4221 | 0 |
| VX-222 | −0.01 | 4222 | 0 |
| adaprev | −0.01 | 4223 | 0 |
| lithium-citrate | −0.01 | 4224 | 0 |
| vildagliptin | −0.01 | 4225 | 0 |
| org-9768 | −0.01 | 4226 | 0 |
| ASP3026 | −0.01 | 4227 | 0 |
| scopine | −0.01 | 4228 | 0 |
| oxaloacetate | −0.01 | 4229 | 0 |
| SB-334867 | −0.01 | 4230 | 0 |
| colfosceril-palmitate | −0.01 | 4231 | 0 |
| FH1 | −0.01 | 4232 | 0 |
| zolmitriptan | −0.01 | 4233 | 0 |
| r(−)-apomorphine | −0.01 | 4234 | 0 |
| perzinfotel | −0.01 | 4235 | 0 |
| VU0361737 | −0.01 | 4236 | 0 |
| thioperamide | −0.01 | 4237 | 0 |
| neostigmine | −0.01 | 4238 | 0 |
| AM-251 | −0.01 | 4239 | 0 |
| ethacridine-lactate-monohydrate | −0.01 | 4240 | 0 |
| daclatasvir | −0.01 | 4241 | 0 |
| FR-139317 | −0.01 | 4242 | 0 |
| SB-216763 | −0.01 | 4243 | 0 |
| guanaben-acetate | −0.01 | 4244 | 0 |
| argatroban | −0.01 | 4245 | 0 |
| dextroamphetamine | −0.01 | 4246 | 0 |
| iodipamide | −0.01 | 4247 | 0 |
| epinephrine | −0.01 | 4248 | 0 |
| tolfenamic-acid | −0.01 | 4249 | 0 |
| neohesperidin | −0.01 | 4250 | 0 |
| cortisone-acetate | −0.01 | 4251 | 0 |
| tagatose | −0.01 | 4252 | 0 |
| cisapride | −0.01 | 4253 | 0 |
| myricetin | −0.01 | 4254 | 0 |
| dihydromyricetin | −0.01 | 4255 | 0 |
| fursultiamine | −0.01 | 4256 | 0 |
| RS-100329 | −0.01 | 4257 | 0 |
| letermovir | −0.01 | 4258 | 0 |
| bisoprolol | −0.01 | 4259 | 0 |
| fostamatinib | −0.01 | 4260 | 0 |
| cyanocobalamin | −0.01 | 4261 | 0 |
| tramiprosate | −0.01 | 4262 | 0 |
| alarelin | −0.01 | 4263 | 0 |
| zileuton | −0.01 | 4264 | 0 |
| methiopril | −0.01 | 4265 | 0 |
| picolinic-acid | −0.01 | 4266 | 0 |
| A-674563 | −0.01 | 4267 | 0 |
| azilsartan-medoxomil | −0.01 | 4268 | 0 |
| cariporide | −0.01 | 4269 | 0 |
| hydralazine | −0.01 | 4270 | 0 |
| androstenone | −0.01 | 4271 | 0 |
| omeprazole-magnesium | −0.01 | 4272 | 0 |
| xylose | −0.01 | 4273 | 0 |
| nalmefene | −0.01 | 4274 | 0 |
| prasugrel | −0.01 | 4275 | 0 |
| PCA-4248 | −0.01 | 4276 | 0 |
| oleanolic-acid | −0.01 | 4277 | 0 |
| forodesine | −0.01 | 4278 | 0 |
| bipenamol | −0.01 | 4279 | 0 |
| fludroxycortide | −0.01 | 4280 | 0 |
| lomefloxacin | −0.01 | 4281 | 0 |
| peretinoin | −0.01 | 4282 | 0 |
| clofibric-acid | −0.01 | 4283 | 0 |
| cefdinir | −0.01 | 4284 | 0 |
| captamine | −0.01 | 4285 | 0 |
| MDL-72832 | −0.01 | 4286 | 0 |
| cariprazine | −0.01 | 4287 | 0 |
| albendazole-oxide | −0.01 | 4288 | 0 |
| sucralose | −0.01 | 4289 | 0 |
| copanlisib | −0.01 | 4290 | 0 |
| phenelzine | −0.01 | 4291 | 0 |
| cilostazol | −0.01 | 4292 | 0 |
| acetophenazine | −0.01 | 4293 | 0 |
| pronethalol | −0.01 | 4294 | 0 |
| ipidacrine | −0.01 | 4295 | 0 |
| nalidixic-acid | −0.01 | 4296 | 0 |
| se-methylselenocysteine | −0.01 | 4297 | 0 |
| desvenlafaxine | −0.01 | 4298 | 0 |
| fenaclon | −0.01 | 4299 | 0 |
| indirubin | −0.01 | 4300 | 0 |
| doconexent-ethyl-ester | −0.01 | 4301 | 0 |
| dexfosfoserine | −0.01 | 4302 | 0 |
| norepinephrine | −0.01 | 4303 | 0 |
| clocortolone-pivalate | −0.01 | 4304 | 0 |
| kil6198 | −0.01 | 4305 | 0 |
| cyclamic-acid | −0.01 | 4306 | 0 |
| ginkgolide-a | −0.01 | 4307 | 0 |
| fendiline | −0.01 | 4308 | 0 |
| candesartan-cilextil | −0.01 | 4309 | 0 |
| cis-urocanic-acid | −0.01 | 4310 | 0 |
| glipizide | −0.01 | 4311 | 0 |
| batimastat | −0.01 | 4312 | 0 |
| LY2603618 | −0.01 | 4313 | 0 |
| mevalonic-d,-1-acid-lactone | −0.01 | 4314 | 0 |
| nitromide | −0.01 | 4315 | 0 |
| tropisetron | −0.01 | 4316 | 0 |
| gavestinel | −0.01 | 4317 | 0 |
| HC-030031 | −0.01 | 4318 | 0 |
| cinnarazine | −0.01 | 4319 | 0 |
| estradiol-cypionate | −0.01 | 4320 | 0 |
| hexestrol | −0.01 | 4321 | 0 |
| clonidine | −0.01 | 4322 | 0 |
| AZD5069 | −0.01 | 4323 | 0 |
| anisotropine | −0.01 | 4324 | 0 |
| TIC10 | −0.01 | 4325 | 0 |
| 5-hydroxymethyl-tolterodine | −0.01 | 4326 | 0 |
| rolipram-S(+) | −0.01 | 4327 | 0 |
| saclofen | −0.01 | 4328 | 0 |
| 8-hydroxy-DPAT | −0.01 | 4329 | 0 |
| nafcillin | −0.01 | 4330 | 0 |
| VU0364439 | −0.01 | 4331 | 0 |
| loratadine | −0.01 | 4332 | 0 |
| tetrahydropapaverine | −0.01 | 4333 | 0 |
| NU-7026 | −0.01 | 4334 | 0 |
| Ro-20-1724 | −0.01 | 4335 | 0 |
| triciribine | −0.01 | 4336 | 0 |
| MNITMT | −0.01 | 4337 | 0 |
| pelanserin | −0.01 | 4338 | 0 |
| proguanil | −0.01 | 4339 | 0 |
| bendroflumethiazide | −0.01 | 4340 | 0 |
| beta-CCB | −0.01 | 4341 | 0 |
| triciribine | −0.01 | 4342 | 0 |
| tryptanthrin | −0.01 | 4343 | 0 |
| creatinol-phosphate | −0.01 | 4344 | 0 |
| adenine | −0.01 | 4345 | 0 |
| agomelatine | −0.01 | 4346 | 0 |
| cytisine | −0.01 | 4347 | 0 |
| sotagliflozin | −0.01 | 4348 | 0 |
| oxiconazole | −0.01 | 4349 | 0 |
| crenolanib | −0.01 | 4350 | 0 |
| corosolic-acid | −0.01 | 4351 | 0 |
| lamotrigine | −0.01 | 4352 | 0 |
| SPP301 | −0.01 | 4353 | 0 |
| 1s,2r-phenylpropanolamine | −0.01 | 4354 | 0 |
| zotepine | −0.01 | 4355 | 0 |
| GSK256066 | −0.01 | 4356 | 0 |
| AST-1306 | −0.01 | 4357 | 0 |
| PSB-06126 | −0.01 | 4358 | 0 |
| troxerutin | −0.01 | 4359 | 0 |
| choline-alfoscerate | −0.01 | 4360 | 0 |
| metoxibutropate | −0.01 | 4361 | 0 |
| menbutone | −0.01 | 4362 | 0 |
| ZD-7288 | −0.01 | 4363 | 0 |
| nifurtimox | −0.01 | 4364 | 0 |
| clofoctol | −0.01 | 4365 | 0 |
| ethyl-paraben | −0.01 | 4366 | 0 |
| RG108 | −0.01 | 4367 | 0 |
| BGT226 | −0.01 | 4368 | 0 |
| safinamide | −0.01 | 4369 | 0 |
| dutasteride | −0.01 | 4370 | 0 |
| eloperastine | −0.01 | 4371 | 0 |
| mevastatin | −0.01 | 4372 | 0 |
| pyritinol | −0.01 | 4373 | 0 |
| nilvadipine | −0.01 | 4374 | 0 |
| CGP-54626 | −0.01 | 4375 | 0 |
| caffeic-acid | −0.01 | 4376 | 0 |
| chlorotrianisene | −0.01 | 4377 | 0 |
| retigabine | −0.01 | 4378 | 0 |
| nesbuvir | −0.01 | 4379 | 0 |
| ambrisentan | −0.01 | 4380 | 0 |
| azomycin-(2-nitroimidazole) | −0.01 | 4381 | 0 |
| aztreonam | −0.01 | 4382 | 0 |
| fudosteine | −0.01 | 4383 | 0 |
| PP-1 | −0.01 | 4384 | 0 |
| fenticonazole | −0.01 | 4385 | 0 |
| bismuth- [symbol] potassium | −0.01 | 4386 | 0 |
| PI3K-IN-2 | −0.01 | 4387 | 0 |
| FPL-62064 | −0.01 | 4388 | 0 |
| picolamine | −0.01 | 4389 | 0 |
| androsterone | −0.01 | 4390 | 0 |
| trapidil | −0.01 | 4391 | 0 |
| ICI-63197 | −0.01 | 4392 | 0 |
| TCI | −0.01 | 4393 | 0 |
| SRPIN340 | −0.01 | 4394 | 0 |
| WZ8040 | −0.01 | 4395 | 0 |
| isobutamben | −0.01 | 4396 | 0 |
| cytarabine | −0.01 | 4397 | 0 |
| OAC1 | −0.01 | 4398 | 0 |
| tolterodine | −0.01 | 4399 | 0 |
| diosmin | −0.01 | 4400 | 0 |
| LY2365109 | −0.01 | 4401 | 0 |
| resibufogenin | −0.01 | 4402 | 0 |
| hydroxyfasudil | −0.01 | 4403 | 0 |
| clotiapine | −0.01 | 4404 | 0 |
| dihydroergotamine | −0.01 | 4405 | 0 |
| promethazine | −0.01 | 4406 | 0 |
| butenafine | −0.01 | 4407 | 0 |
| vandetanib | −0.01 | 4408 | 0 |
| fleroxacin | −0.01 | 4409 | 0 |
| Y-27152 | −0.01 | 4410 | 0 |
| ICI-89406 | −0.01 | 4411 | 0 |
| fesoterodine | −0.01 | 4412 | 0 |
| guanosine | −0.01 | 4413 | 0 |
| OSI-420 | −0.01 | 4414 | 0 |
| dilazep | −0.01 | 4415 | 0 |
| coumarin | −0.01 | 4416 | 0 |
| icosapent | −0.01 | 4417 | 0 |
| indacaterol | −0.01 | 4418 | 0 |
| 4-iodo-6-phenylpyrimidine | −0.01 | 4419 | 0 |
| ku-0063794 | −0.01 | 4420 | 0 |
| ceftiofur | −0.01 | 4421 | 0 |
| L-655240 | −0.01 | 4422 | 0 |
| pritelivir | −0.01 | 4423 | 0 |
| 1-azakenpaullone | −0.01 | 4424 | 0 |
| oglemilast | −0.01 | 4425 | 0 |
| QX-314 | −0.01 | 4426 | 0 |
| C-751 | −0.01 | 4427 | 0 |
| PF-04937319 | −0.01 | 4428 | 0 |
| 3-deazaadenosine | −0.01 | 4429 | 0 |
| 16,16-dimethylprostaglandin-e2 | −0.01 | 4430 | 0 |
| chlormezanone | −0.01 | 4431 | 0 |
| levodropropizine | −0.01 | 4432 | 0 |
| rutin | −0.01 | 4433 | 0 |
| icotinib | −0.01 | 4434 | 0 |
| geniposide | −0.01 | 4435 | 0 |
| azacyclonol | −0.01 | 4436 | 0 |
| carboplatin | −0.01 | 4437 | 0 |
| selumetinib | −0.01 | 4438 | 0 |
| osthol | −0.01 | 4439 | 0 |
| inositol-hexanicotinate | −0.01 | 4440 | 0 |
| orteronel | −0.01 | 4441 | 0 |
| pentoxifylline | −0.01 | 4442 | 0 |
| RTA-408 | −0.01 | 4443 | 0 |
| temozolomide | −0.01 | 4444 | 0 |
| baicalin | −0.01 | 4445 | 0 |
| purvalanol-a | −0.01 | 4446 | 0 |
| trelagliptin | −0.01 | 4447 | 0 |
| trandolapril | −0.01 | 4448 | 0 |
| perfluamine | −0.01 | 4449 | 0 |
| phosphatidylcholine | −0.01 | 4450 | 0 |
| danazol | −0.01 | 4451 | 0 |
| doxifluridine | −0.01 | 4452 | 0 |
| tetrandrine | −0.01 | 4453 | 0 |
| tezacaftor | −0.01 | 4454 | 0 |
| propranolol-(S) | −0.01 | 4455 | 0 |
| iodoantipyrine | −0.01 | 4456 | 0 |
| dantrolene | −0.01 | 4457 | 0 |
| lersivirine | −0.01 | 4458 | 0 |
| tadalafil | −0.01 | 4459 | 0 |
| bromocriptine | −0.01 | 4460 | 0 |
| cilomilast | −0.01 | 4461 | 0 |
| etebenecid | −0.01 | 4462 | 0 |
| norgestrel | −0.01 | 4463 | 0 |
| tracazolate | −0.01 | 4464 | 0 |
| rufinamide | −0.01 | 4465 | 0 |
| daphnetin | −0.01 | 4466 | 0 |
| INCB-3284 | −0.01 | 4467 | 0 |
| metoclopramide | −0.01 | 4468 | 0 |
| 2-cyanopyrimidine | −0.01 | 4469 | 0 |
| XL888 | −0.01 | 4470 | 0 |
| elacridar | −0.01 | 4471 | 0 |
| LY456236 | −0.01 | 4472 | 0 |
| ST-91 | −0.01 | 4473 | 0 |
| probucol | −0.02 | 4474 | 0 |
| zolimidine | −0.02 | 4475 | 0 |
| parecoxib | −0.02 | 4476 | 0 |
| diacetamate | −0.02 | 4477 | 0 |
| NVP-AUY922 | −0.02 | 4478 | 0 |
| Y-26763 | −0.02 | 4479 | 0 |
| simvastatin | −0.02 | 4480 | 0 |
| bedaquiline | −0.02 | 4481 | 0 |
| chromocarb | −0.02 | 4482 | 0 |
| FK-866 | −0.02 | 4483 | 0 |
| parachlorophenol | −0.02 | 4484 | 0 |
| ixazomib-citrate | −0.02 | 4485 | 0 |
| neridronic-acid | −0.02 | 4486 | 0 |
| 2,6-dimethylpiperidine | −0.02 | 4487 | 0 |
| I-BZA2 | −0.02 | 4488 | 0 |
| RI-1 | −0.02 | 4489 | 0 |
| Ixabepilone | −0.02 | 4490 | 0 |
| roquinimex | −0.02 | 4491 | 0 |
| colistin-b-sulfate | −0.02 | 4492 | 0 |
| U-104 | −0.02 | 4493 | 0 |
| RN-1734 | −0.02 | 4494 | 0 |
| flumazenil | −0.02 | 4495 | 0 |
| SCH-58261 | −0.02 | 4496 | 0 |
| levonorgestrel | −0.02 | 4497 | 0 |
| RITA | −0.02 | 4498 | 0 |
| topiramate | −0.02 | 4499 | 0 |
| masoprocol | −0.02 | 4500 | 0 |
| SB-218795 | −0.02 | 4501 | 0 |
| AS-604850 | −0.02 | 4502 | 0 |
| urethane | −0.02 | 4503 | 0 |
| JNJ-10191584 | −0.02 | 4504 | 0 |
| omeprazole-sulfide | −0.02 | 4505 | 0 |
| orantinib | −0.02 | 4506 | 0 |
| linifanib | −0.02 | 4507 | 0 |
| 1,5-dicaffeoylquinic-acid | −0.02 | 4508 | 0 |
| ivacaftor | −0.02 | 4509 | 0 |
| 7-nitroindazole | −0.02 | 4510 | 0 |
| m-THP | −0.02 | 4511 | 0 |
| dicoumarol | −0.02 | 4512 | 0 |
| BMS-986020 | −0.02 | 4513 | 0 |
| dipyridamole | −0.02 | 4514 | 0 |
| PJ-34 | −0.02 | 4515 | 0 |
| dichlorvos | −0.02 | 4516 | 0 |
| mezlocillin | −0.02 | 4517 | 0 |
| ethimizol | −0.02 | 4518 | 0 |
| peruvoside | −0.02 | 4519 | 0 |
| carbetapentane | −0.02 | 4520 | 0 |
| BAM7 | −0.02 | 4521 | 0 |
| ponatinib | −0.02 | 4522 | 0 |
| sangivamycin | −0.02 | 4523 | 0 |
| ronidazole | −0.02 | 4524 | 0 |
| repaglinide | −0.02 | 4525 | 0 |
| fluvoxamine | −0.02 | 4526 | 0 |
| acetyl-11-keto-beta-boswellic-acid | −0.02 | 4527 | 0 |
| 2,3-DCPE | −0.02 | 4528 | 0 |
| tesaglitazar | −0.02 | 4529 | 0 |
| fleroxacin | −0.02 | 4530 | 0 |
| P5091 | −0.02 | 4531 | 0 |
| AV-412 | −0.02 | 4532 | 0 |
| clopamide | −0.02 | 4533 | 0 |
| alizarin | −0.02 | 4534 | 0 |
| inosine | −0.02 | 4535 | 0 |
| bis(maltolato)oxovanadium(IV) | −0.02 | 4536 | 0 |
| necrostatin-1 | −0.02 | 4537 | 0 |
| DVD-111 | −0.02 | 4538 | 0 |
| ornithine | −0.02 | 4539 | 0 |
| alendronate | −0.02 | 4540 | 0 |
| PSB-11 | −0.02 | 4541 | 0 |
| glycopyrrolate | −0.02 | 4542 | 0 |
| dibenzothiophene | −0.02 | 4543 | 0 |
| chlorindanol | −0.02 | 4544 | 0 |
| thiamine | −0.02 | 4545 | 0 |
| BRD4770 | −0.02 | 4546 | 0 |
| ZAPA | −0.02 | 4547 | 0 |
| ouabain | −0.02 | 4548 | 0 |
| hexylcaine | −0.02 | 4549 | 0 |
| meglitinide | −0.02 | 4550 | 0 |
| clinofibrate | −0.02 | 4551 | 0 |
| SD-169 | −0.02 | 4552 | 0 |
| voxtalisib | −0.02 | 4553 | 0 |
| creatine | −0.02 | 4554 | 0 |
| lithocholie-acid | −0.02 | 4555 | 0 |
| PNU-37883 | −0.02 | 4556 | 0 |
| CCMI | −0.02 | 4557 | 0 |
| rosuvastatin | −0.02 | 4558 | 0 |
| AMG-232 | −0.02 | 4559 | 0 |
| epiandrosterone | −0.02 | 4560 | 0 |
| sodium-gualenate | −0.02 | 4561 | 0 |
| dasatinib | −0.02 | 4562 | 0 |
| amrinone | −0.02 | 4563 | 0 |
| dacinostat | −0.02 | 4564 | 0 |
| IDRA-21 | −0.02 | 4565 | 0 |
| hypericin | −0.02 | 4566 | 0 |
| saracatinib | −0.02 | 4567 | 0 |
| PP-121 | −0.02 | 4568 | 0 |
| fludarabine | −0.02 | 4569 | 0 |
| phenazone | −0.02 | 4570 | 0 |
| chloroxine | −0.02 | 4571 | 0 |
| centpropazine | −0.02 | 4572 | 0 |
| SIB-1757 | −0.02 | 4573 | 0 |
| sulfasalazine | −0.02 | 4574 | 0 |
| MPEP | −0.02 | 4575 | 0 |
| fosfomycin | −0.02 | 4576 | 0 |
| GW-1929 | −0.02 | 4577 | 0 |
| siguazodan | −0.02 | 4578 | 0 |
| oleamide | −0.02 | 4579 | 0 |
| perillyl-alcohol | −0.02 | 4580 | 0 |
| AZD5438 | −0.02 | 4581 | 0 |
| DCEBIO | −0.02 | 4582 | 0 |
| EGF816 | −0.02 | 4583 | 0 |
| 5,7-dichlorokynurenic-acid | −0.02 | 4584 | 0 |
| AZD8931 | −0.02 | 4585 | 0 |
| ethosuximide | −0.02 | 4586 | 0 |
| brigatinib | −0.02 | 4587 | 0 |
| hesperadin | −0.02 | 4588 | 0 |
| AT13387 | −0.02 | 4589 | 0 |
| teneligliptin | −0.02 | 4590 | 0 |
| naftidrofuryl | −0.02 | 4591 | 0 |
| fentiazac | −0.02 | 4592 | 0 |
| docetaxel | −0.02 | 4593 | 0 |
| pelitinib | −0.02 | 4594 | 0 |
| bunazosin | −0.02 | 4595 | 0 |
| torin-1 | −0.02 | 4596 | 0 |
| genipin | −0.02 | 4597 | 0 |
| LY364947 | −0.02 | 4598 | 0 |
| telotristat | −0.02 | 4599 | 0 |
| pomalidomide | −0.02 | 4600 | 0 |
| equol | −0.02 | 4601 | 0 |
| EMD-386088 | −0.02 | 4602 | 0 |
| editol | −0.02 | 4603 | 0 |
| ritanserin | −0.02 | 4604 | 0 |
| gilteritinib | −0.02 | 4605 | 0 |
| chromanol-(+/−) | −0.02 | 4606 | 0 |
| AV-608 | −0.02 | 4607 | 0 |
| LY2157299 | −0.02 | 4608 | 0 |
| PD-123319 | −0.02 | 4609 | 0 |
| CGS-15943 | −0.02 | 4610 | 0 |
| brilliant-green | −0.02 | 4611 | 0 |
| latrepirdine | −0.02 | 4612 | 0 |
| bosentan | −0.02 | 4613 | 0 |
| NU6027 | −0.02 | 4614 | 0 |
| estradiol | −0.02 | 4615 | 0 |
| erlotinib | −0.02 | 4616 | 0 |
| GDC-0152 | −0.02 | 4617 | 0 |
| D-7193 | −0.02 | 4618 | 0 |
| tecastemizole | −0.02 | 4619 | 0 |
| R306465 | −0.02 | 4620 | 0 |
| ozagrel | −0.02 | 4621 | 0 |
| MRS-1220 | −0.02 | 4622 | 0 |
| PD-318088 | −0.02 | 4623 | 0 |
| AP26113 | −0.02 | 4624 | 0 |
| PD-198306 | −0.02 | 4625 | 0 |
| CHIR-98014 | −0.02 | 4626 | 0 |
| efonidip [symbol] monoethanolate | −0.02 | 4627 | 0 |
| geniposidic-acid | −0.02 | 4628 | 0 |
| ER-27319 | −0.02 | 4629 | 0 |
| exatecan-mesylate | −0.02 | 4630 | 0 |
| MEK162 | −0.02 | 4631 | 0 |
| kenpaullone | −0.02 | 4632 | 0 |
| diarylpropionitrile | −0.02 | 4633 | 0 |
| sodium-tetradecyl-sulfate | −0.02 | 4634 | 0 |
| batimastat | −0.02 | 4635 | 0 |
| oxymatrine | −0.02 | 4636 | 0 |
| forskolin | −0.02 | 4637 | 0 |
| CCT137690 | −0.02 | 4638 | 0 |
| BVD-523 | −0.03 | 4639 | 0 |
| alvespimycin | −0.03 | 4640 | 0 |
| WP1066 | −0.03 | 4641 | 0 |
| pantothenic-acid | −0.03 | 4642 | 0 |
| PP-2 | −0.03 | 4643 | 0 |
| DRF053-(R) | −0.03 | 4644 | 0 |
| erythromycin-ethylsuccinate | −0.03 | 4645 | 0 |
| beta-naphthol | −0.03 | 4646 | 0 |
| PHA-767491 | −0.03 | 4647 | 0 |
| noretynodrel | −0.03 | 4648 | 0 |
| riociguat | −0.03 | 4649 | 0 |
| levocetirizine | −0.03 | 4650 | 0 |
| cromoglicic-acid | −0.03 | 4651 | 0 |
| salidroside | −0.03 | 4652 | 0 |
| birinapant | −0.03 | 4653 | 0 |
| atorvastatin | −0.03 | 4654 | 0 |
| GW-441756 | −0.03 | 4655 | 0 |
| acivicin | −0.03 | 4656 | 0 |
| PD-168393 | −0.03 | 4657 | 0 |
| LCL-161 | −0.03 | 4658 | 0 |
| R547 | −0.03 | 4659 | 0 |
| rutin | −0.03 | 4660 | 0 |
| NVP-BVU972 | −0.03 | 4661 | 0 |
| P22077 | −0.03 | 4662 | 0 |
| doxorubicin | −0.03 | 4663 | 0 |
| phentolamine | −0.03 | 4664 | 0 |
| trametinib | −0.03 | 4665 | 0 |
| latanoprost | −0.03 | 4666 | 0 |
| R406 | −0.03 | 4667 | 0 |
| tosedostat | −0.03 | 4668 | 0 |
| PHA-665752 | −0.04 | 4669 | 0 |
| CR8-(R) | −0.04 | 4670 | 0 |
| BMS-690514 | −0.04 | 4671 | 0 |
| delanzomib | −0.04 | 4672 | 0 |
| FG-4592 | −0.04 | 4673 | 0 |
| poziotinib | −0.04 | 4674 | 0 |
| epirubicin | −0.04 | 4675 | 0 |
| BMS-387032 | −0.05 | 4676 | 0 |
| cobimetinib | −0.05 | 4677 | 0 |
| AS-703026 | −0.05 | 4678 | 0 |
| Ro-4987655 | −0.05 | 4679 | 0 |
| AT-7519 | −0.05 | 4680 | 0 |
| mevastatin | −0.05 | 4681 | 0 |
| MG-132 | −0.06 | 4682 | 0 |
| TAK-733 | −0.06 | 4683 | 0 |
| PD-0325901 | −0.07 | 4684 | 0 |
| AZD8330 | −0.07 | 4685 | 0 |
| ganetespib | −0.07 | 4686 | 0 |

[symbol]

indicates data missing or illegible when filed

TABLE 5A
GDSC-based Drug target classes with sensitivity in SCN, SRBCT or Blood cancer types

| | | | | | | | | SCN | SRBCT | SCN | Sensitivity |
|---|---|---|---|---|---|---|---|---|---|---|---|
| | | | | | | | | & | & | & | in |
| | | SCN - SCLC | | | | | | Blood | Blood | SRBCT | SCN, |
| | | vs LUAD | | SRBCT vs | | Blood vs | | avg. | avg. | avg | SRBCT |
| drug target | | lung cancer | | OTHER | | OTHER | | Avg. | Avg. | Avg. | or |
| target | freq | signlogp | rank | signlogp | rank | signlogp | rank | rank | rank | rank | Blood |
| HDAC1 | 9 | 2.31 | 1 | 0.65 | 7 | 1.16 | 2 | 1.5 | 4.5 | 4 | 1 |
| ROCK2 | 3 | 1.22 | 2 | 0.32 | 12 | 0.48 | 14 | 8 | 13 | 7 | 1 |
| AURKA | 4 | 1.15 | 3 | 0.53 | 10 | 0.07 | 21 | 12 | 15.5 | 6.5 | 1 |
| CDK7 | 5 | 0.86 | 4 | −0.15 | 29 | 0.98 | 3 | 3.5 | 16 | 16.5 | 1 |
| AURKB | 5 | 0.82 | 5 | 0.31 | 13 | 0.13 | 19 | 12 | 16 | 9 | 1 |
| MET | 5 | 0.71 | 6 | 0.43 | 11 | 0.31 | 16 | 11 | 13.5 | 8.5 | 1 |
| BTK | 4 | 0.66 | 7 | 0.82 | 4 | 0.67 | 8 | 7.5 | 6 | 5.5 | 1 |
| ALK | 7 | 0.64 | 8 | 0.70 | 6 | 0.71 | 7 | 7.5 | 6.5 | 7 | 1 |
| JAK2 | 3 | 0.63 | 9 | 0.99 | 3 | 0.97 | 4 | 6.5 | 3.5 | 6 | 1 |
| CDK2 | 4 | 0.56 | 10 | −0.33 | 34 | 0.50 | 12 | 11 | 23 | 22 | 1 |
| FLT3 | 8 | 0.56 | 11 | 0.54 | 9 | 1.50 | 1 | 6 | 5 | 10 | 1 |
| AURKC | 3 | 0.40 | 12 | 0.05 | 17 | −0.05 | 24 | 18 | 20.5 | 14.5 | 1 |
| VEGFR2 | 3 | 0.39 | 13 | 0.01 | 22 | 0.49 | 13 | 13 | 17.5 | 17.5 | 1 |
| OTHERS | 4 | 0.30 | 14 | 0.02 | 21 | 0.32 | 15 | 14.5 | 18 | 17.5 | 1 |
| PDGFRA | 4 | 0.29 | 15 | −0.07 | 27 | 0.09 | 20 | 17.5 | 23.5 | 21 | 1 |
| KIT | 12 | 0.29 | 16 | 0.05 | 18 | −0.45 | 35 | 25.5 | 26.5 | 17 | 1 |
| FGFR1 | 3 | 0.26 | 17 | −0.06 | 25 | 0.59 | 10 | 13.5 | 17.5 | 21 | 1 |
| BMX | 3 | 0.22 | 18 | −0.38 | 35 | 0.00 | 22 | 20 | 28.5 | 26.5 | 1 |
| CSF1R | 4 | 0.20 | 19 | −0.42 | 37 | −0.31 | 32 | 25.5 | 34.5 | 28 | 1 |
| PDK1 | 4 | 0.18 | 21 | 0.70 | 5 | 0.66 | 9 | 15 | 7 | 13 | 1 |
| BRD4 | 3 | 0.16 | 22 | 0.12 | 16 | 0.85 | 5 | 13.5 | 10.5 | 19 | 1 |
| MTORC2 | 4 | 0.09 | 25 | −0.07 | 26 | 0.51 | 11 | 18 | 18.5 | 25.5 | 1 |
| CDK9 | 5 | 0.08 | 26 | −0.15 | 29 | 0.82 | 6 | 16 | 17.5 | 27.5 | 1 |
| PARP1 | 4 | −0.15 | 34 | 1.51 | 1 | −0.14 | 27 | 30.5 | 14 | 17.5 | 1 |
| PARP2 | 4 | −0.15 | 34 | 1.51 | 1 | −0.14 | 27 | 30.5 | 14 | 17.5 | 1 |
| IGF1R | 4 | −0.53 | 41 | 0.62 | 8 | −1.53 | 46 | 43.5 | 27 | 24.5 | 1 |
| VEGFR | 9 | 0.20 | 20 | 0.17 | 14 | −0.53 | 38 | 29 | 26 | 17 | 0 |
| BCL-XL | 3 | 0.10 | 23 | 0.03 | 19 | −0.05 | 25 | 24 | 22 | 21 | 0 |
| BCL2 | 3 | 0.10 | 23 | 0.03 | 19 | −0.05 | 25 | 24 | 22 | 21 | 0 |
| AKT3 | 4 | 0.07 | 27 | −0.38 | 36 | −0.24 | 31 | 29 | 33.5 | 31.5 | 0 |
| PDGFR | 11 | 0.01 | 28 | −0.03 | 23 | −0.83 | 43 | 35.5 | 33 | 25.5 | 0 |
| IR | 4 | 0.01 | 29 | 0.16 | 15 | −0.40 | 34 | 31.5 | 24.5 | 22 | 0 |
| AKT1 | 5 | −0.01 | 30 | −0.48 | 38 | −0.23 | 29 | 29.5 | 33.5 | 34 | 0 |
| AKT2 | 5 | −0.01 | 30 | −0.48 | 38 | −0.23 | 29 | 29.5 | 33.5 | 34 | 0 |
| CDK4 | 3 | −0.06 | 32 | −0.04 | 24 | −0.03 | 23 | 27.5 | 23.5 | 28 | 0 |
| MTORC1 | 5 | −0.06 | 33 | −0.14 | 28 | 0.16 | 18 | 25.5 | 23 | 30.5 | 0 |
| PI3K | 11 | −0.18 | 36 | −0.27 | 32 | 0.19 | 17 | 26.5 | 24.5 | 34 | 0 |
| LCK | 3 | −0.31 | 37 | −0.97 | 43 | −0.74 | 41 | 39 | 42 | 40 | 0 |
| BRAF | 6 | −0.37 | 38 | −2.84 | 49 | −1.89 | 48 | 43 | 48.5 | 43.5 | 0 |
| ERBB2 | 5 | −0.45 | 39 | −1.22 | 45 | −1.22 | 45 | 42 | 45 | 42 | 0 |
| RET | 6 | −0.53 | 40 | −0.32 | 33 | −0.48 | 36 | 38 | 34.5 | 36.5 | 0 |
| MICROTUBULE | 3 | −0.60 | 42 | −1.47 | 46 | −2.57 | 49 | 45.5 | 47.5 | 44 | 0 |
| STABILISER | | | | | | | | | | | |
| MTOR | 8 | −0.94 | 43 | −1.20 | 44 | −0.49 | 37 | 40 | 40.5 | 43.5 | 0 |
| JNK2 | 3 | −0.97 | 44 | −0.58 | 40 | −0.80 | 42 | 43 | 41 | 42 | 0 |
| PKC | 4 | −1.14 | 45 | −2.19 | 48 | −0.39 | 33 | 39 | 40.5 | 46.5 | 0 |
| ABL | 7 | −1.19 | 46 | −0.65 | 41 | −1.01 | 44 | 45 | 42.5 | 43.5 | 0 |
| HSP90 | 5 | −1.60 | 47 | −0.26 | 31 | −0.54 | 39 | 43 | 35 | 39 | 0 |
| SRC | 6 | −1.76 | 48 | −1.94 | 47 | −0.63 | 40 | 44 | 43.5 | 47.5 | 0 |
| EGFR | 10 | −3.14 | 49 | −0.89 | 42 | −1.82 | 47 | 48 | 44.5 | 45.5 | 0 |
| MEK1 | 6 | −8.59 | 50 | −5.68 | 50 | −6.38 | 50 | 50 | 50 | 50 | 0 |
| MEK2 | 6 | −8.59 | 50 | −5.68 | 50 | −6.38 | 50 | 50 | 50 | 50 | 0 |

TABLE 5B
PRISM-based Drug target classes with sensitivity
in SCN, SRBCT or Blood cancer types

| | | SCN - SCLC vs LUAD lung cancer | | | | |
|---|---|---|---|---|---|---|
| | | | | signlogp | | |
| | | | | (pos = more | | |
| drug targets | | | | sensitive | | Sensitivity |
| target | freq | pval | ES | in SCLC) | rank | in SCN |
| TUBB | 26 | 5.51E−14 | 0.78 | 13.26 | 1 | 1 |
| AURKA | 23 | 3.71E−11 | 0.73 | 10.43 | 2 | 1 |
| HDAC1 | 29 | 1.41E−08 | 0.57 | 7.85 | 3 | 1 |
| HDAC3 | 20 | 9.60E−08 | 0.65 | 7.02 | 4 | 1 |
| AURKB | 22 | 4.83E−07 | 0.59 | 6.32 | 5 | 1 |
| HDAC6 | 17 | 6.59E−06 | 0.61 | 5.18 | 6 | 1 |
| BRD4 | 9 | 5.32E−05 | 0.77 | 4.27 | 7 | 1 |
| IMPDH1 | 9 | 8.28E−05 | 0.75 | 4.08 | 8 | 1 |
| HDAC5 | 10 | 1.16E−04 | 0.70 | 3.94 | 9 | 1 |
| HDAC8 | 14 | 1.41E−04 | 0.59 | 3.85 | 10 | 1 |
| AURKC | 10 | 2.67E−04 | 0.67 | 3.57 | 11 | 1 |
| BCL2 | 16 | 4.35E−04 | 0.51 | 3.36 | 12 | 1 |
| HDAC9 | 13 | 4.59E−04 | 0.57 | 3.34 | 13 | 1 |
| HDAC4 | 11 | 4.66E−04 | 0.62 | 3.33 | 14 | 1 |
| NR3C1 | 66 | 4.74E−04 | 0.25 | 3.32 | 15 | 1 |
| TUBB1 | 10 | 7.65E−04 | 0.63 | 3.12 | 16 | 1 |
| HDAC2 | 18 | 8.25E−04 | 0.47 | 3.08 | 17 | 1 |
| TOP2A | 34 | 8.63E−04 | 0.34 | 3.06 | 18 | 1 |
| MTOR | 38 | 1.51E−03 | 0.31 | 2.82 | 19 | 1 |
| MET | 26 | 2.17E−03 | 0.36 | 2.66 | 20 | 1 |
| TOP1 | 13 | 3.67E−03 | 0.49 | 2.44 | 21 | 1 |
| HDAC10 | 9 | 5.10E−03 | 0.58 | 2.29 | 22 | 1 |
| CHRM5 | 48 | 5.20E−03 | 0.25 | 2.28 | 23 | 1 |
| AKT2 | 9 | 5.32E−03 | 0.57 | 2.27 | 24 | 1 |
| PGR | 38 | 7.02E−03 | 0.27 | 2.15 | 25 | 1 |
| PIK3CD | 25 | 1.57E−02 | 0.31 | 1.81 | 26 | 1 |
| PIK3CG | 31 | 2.77E−02 | 0.26 | 1.56 | 27 | 1 |
| CHRM1 | 108 | 3.63E−02 | 0.14 | 1.44 | 28 | 1 |
| HRH1 | 111 | 4.52E−02 | 0.13 | 1.34 | 29 | 1 |
| BRAF | 14 | 4.79E−02 | 0.37 | 1.32 | 30 | 1 |
| PIK3CA | 29 | 4.81E−02 | 0.25 | 1.32 | 31 | 1 |
| DRD4 | 55 | 4.83E−02 | 0.19 | 1.32 | 32 | 1 |
| CHRM2 | 84 | 5.53E−02 | 0.15 | 1.26 | 33 | 1 |
| FLT4 | 22 | 6.02E−02 | 0.28 | 1.22 | 34 | 1 |
| FLT3 | 27 | 6.13E−02 | 0.25 | 1.21 | 35 | 1 |
| IKBKB | 13 | 6.55E−02 | 0.36 | 1.18 | 36 | 1 |
| KIT | 26 | 7.66E−02 | 0.25 | 1.12 | 37 | 1 |
| SMO | 11 | 7.92E−02 | 0.38 | 1.10 | 38 | 1 |
| ATP1A1 | 16 | 8.04E−02 | 0.32 | 1.09 | 39 | 1 |
| RAF1 | 11 | 8.63E−02 | 0.38 | 1.06 | 40 | 1 |
| JAK2 | 22 | 8.74E−02 | 0.27 | 1.06 | 41 | 1 |
| KDR | 52 | 8.76E−02 | 0.17 | 1.06 | 42 | 1 |
| EHMT2 | 11 | 1.05E−01 | 0.37 | 0.98 | 43 | potential |
| DRD3 | 73 | 1.12E−01 | 0.14 | 0.95 | 44 | potential |
| CHRNA2 | 22 | 1.19E−01 | 0.25 | 0.92 | 45 | potential |
| CSF1R | 13 | 1.25E−01 | 0.33 | 0.90 | 46 | potential |
| DRD5 | 38 | 1.26E−01 | 0.19 | 0.90 | 47 | potential |
| JAK3 | 13 | 1.26E−01 | 0.33 | 0.90 | 48 | potential |
| SYK | 11 | 1.34E−01 | 0.35 | 0.87 | 49 | potential |
| SLC6A4 | 52 | 1.46E−01 | 0.16 | 0.84 | 50 | potential |
| CYP2D6 | 13 | 1.46E−01 | 0.32 | 0.84 | 51 | potential |
| AKT1 | 23 | 1.47E−01 | 0.24 | 0.83 | 52 | potential |
| HTR1E | 32 | 1.53E−01 | 0.20 | 0.81 | 53 | potential |
| SLC12A1 | 12 | 1.57E−01 | 0.33 | 0.81 | 54 | potential |
| DHFR | 11 | 1.57E−01 | 0.34 | 0.80 | 55 | potential |
| CDK2 | 23 | 1.63E−01 | 0.23 | 0.79 | 56 | potential |
| JAK1 | 12 | 1.72E−01 | 0.32 | 0.76 | 57 | potential |
| HTR6 | 51 | 1.81E−01 | 0.15 | 0.74 | 58 | potential |
| MAPK14 | 32 | 1.98E−01 | 0.19 | 0.70 | 59 | potential |
| DRD1 | 65 | 2.06E−01 | 0.13 | 0.69 | 60 | potential |
| HTR2C | 90 | 2.14E−01 | 0.11 | 0.67 | 61 | potential |
| MAPK8 | 13 | 2.22E−01 | 0.29 | 0.65 | 62 | potential |
| PIK3CB | 22 | 2.28E−01 | 0.22 | 0.64 | 63 | potential |
| PRKDC | 9 | 2.31E−01 | 0.35 | 0.64 | 64 | potential |
| CHRM4 | 64 | 2.32E−01 | 0.13 | 0.63 | 65 | potential |
| HTR1B | 55 | 2.40E−01 | 0.14 | 0.62 | 66 | potential |
| HTR2A | 129 | 2.41E−01 | 0.09 | 0.62 | 67 | potential |
| CHEK1 | 19 | 2.47E−01 | 0.24 | 0.61 | 68 | potential |
| DRD2 | 126 | 2.47E−01 | 0.09 | 0.61 | 69 | potential |
| PLA2G1B | 14 | 2.57E−01 | 0.27 | 0.59 | 70 | potential |
| MAPK11 | 16 | 2.60E−01 | 0.25 | 0.58 | 71 | potential |
| TYMS | 18 | 2.72E−01 | 0.24 | 0.56 | 72 | potential |
| FLT1 | 24 | 2.80E−01 | 0.20 | 0.55 | 73 | potential |
| AR | 56 | 2.86E−01 | 0.13 | 0.54 | 74 | potential |
| GABRG2 | 36 | 2.87E−01 | 0.16 | 0.54 | 75 | potential |
| HTR1D | 54 | 2.92E−01 | 0.13 | 0.53 | 76 | potential |
| MMP8 | 10 | 2.94E−01 | 0.31 | 0.53 | 77 | potential |
| CYP3A4 | 48 | 3.10E−01 | 0.14 | 0.51 | 78 | potential |
| BCHE | 16 | 3.15E−01 | 0.24 | 0.50 | 79 | potential |
| FGFR1 | 20 | 3.40E−01 | 0.21 | 0.47 | 80 | potential |
| GABRB1 | 28 | 3.52E−01 | 0.18 | 0.45 | 81 | potential |
| GRM5 | 18 | 3.85E−01 | 0.21 | 0.41 | 82 | potential |
| CHRM3 | 88 | 3.86E−01 | 0.10 | 0.41 | 83 | potential |
| HTR7 | 52 | 3.86E−01 | 0.13 | 0.41 | 84 | potential |
| CHRNA4 | 22 | 3.97E−01 | 0.19 | 0.40 | 85 | potential |
| HTR1A | 116 | 4.01E−01 | 0.08 | 0.40 | 86 | potential |
| XDH | 9 | 4.38E−01 | 0.29 | 0.36 | 87 | potential |
| AVPR1A | 10 | 4.48E−01 | 0.27 | 0.35 | 88 | potential |
| GABRD | 27 | 4.63E−01 | 0.16 | 0.33 | 89 | potential |
| IGF1R | 12 | 4.70E−01 | 0.24 | 0.33 | 90 | potential |
| SLC6A2 | 51 | 4.84E−01 | 0.12 | 0.32 | 91 | potential |
| TRPC5 | 9 | 5.00E−01 | 0.28 | 0.30 | 92 | potential |
| KCNN4 | 16 | 5.01E−01 | 0.21 | 0.30 | 93 | potential |
| NR1I2 | 21 | 5.05E−01 | 0.18 | 0.30 | 94 | potential |
| GABRB3 | 31 | 5.15E−01 | 0.15 | 0.29 | 95 | potential |
| CNR1 | 16 | 5.25E−01 | 0.20 | 0.28 | 96 | potential |
| NTRK1 | 12 | 5.28E−01 | 0.23 | 0.28 | 97 | potential |
| SERPINA6 | 12 | 5.42E−01 | 0.23 | 0.27 | 98 | potential |
| SIGMAR1 | 23 | 5.43E−01 | 0.17 | 0.27 | 99 | potential |
| ROCK1 | 16 | 5.68E−01 | 0.20 | 0.25 | 100 | potential |
| FAAH | 9 | 5.69E−01 | 0.26 | 0.25 | 101 | potential |
| GABRE | 26 | 5.93E−01 | 0.15 | 0.23 | 102 | potential |
| GABRG1 | 26 | 5.93E−01 | 0.15 | 0.23 | 103 | potential |
| GABRG3 | 26 | 5.93E−01 | 0.15 | 0.23 | 104 | potential |
| GABRP | 26 | 5.93E−01 | 0.15 | 0.23 | 105 | potential |
| GABRQ | 26 | 5.93E−01 | 0.15 | 0.23 | 106 | potential |
| PDGFRB | 26 | 5.97E−01 | 0.15 | 0.22 | 107 | potential |
| GABRB2 | 33 | 5.98E−01 | 0.13 | 0.22 | 108 | potential |
| CYP3A5 | 13 | 6.00E−01 | 0.21 | 0.22 | 109 | potential |
| MAPK1 | 41 | 6.01E−01 | 0.12 | 0.22 | 110 | potential |
| MMP13 | 9 | 6.09E−01 | 0.25 | 0.22 | 111 | potential |
| AVPR2 | 10 | 6.22E−01 | 0.24 | 0.21 | 112 | potential |
| CYP2C19 | 13 | 6.29E−01 | 0.21 | 0.20 | 113 | potential |
| DNMT1 | 9 | 6.41E−01 | 0.25 | 0.19 | 114 | potential |
| GABRA5 | 44 | 6.51E−01 | 0.11 | 0.19 | 115 | potential |
| CYP1A2 | 12 | 6.54E−01 | 0.21 | 0.18 | 116 | potential |
| MAPK12 | 11 | 7.36E−01 | 0.21 | 0.13 | 117 | potential |
| CDK4 | 13 | 7.44E−01 | 0.19 | 0.13 | 118 | 0 |
| GABRA3 | 45 | 7.50E−01 | 0.10 | 0.12 | 119 | 0 |
| GRIN2B | 39 | 7.57E−01 | 0.11 | 0.12 | 120 | 0 |
| CYP2B6 | 9 | 7.62E−01 | 0.22 | 0.12 | 121 | 0 |
| HTR5A | 19 | 7.65E−01 | 0.15 | 0.12 | 122 | 0 |
| TYR | 12 | 7.69E−01 | 0.19 | 0.11 | 123 | 0 |
| CYP19A1 | 19 | 7.81E−01 | 0.15 | 0.11 | 124 | 0 |
| NR3C2 | 23 | 7.82E−01 | 0.14 | 0.11 | 125 | 0 |
| GABRA2 | 44 | 7.94E−01 | 0.10 | 0.10 | 126 | 0 |
| SLC12A3 | 14 | 8.29E−01 | 0.17 | 0.08 | 127 | 0 |
| HRH3 | 16 | 8.31E−01 | 0.16 | 0.08 | 128 | 0 |
| KCNH2 | 39 | 8.51E−01 | 0.10 | 0.07 | 129 | 0 |
| GNRHR | 9 | 8.63E−01 | 0.20 | 0.06 | 130 | 0 |
| GRIN2C | 22 | 8.75E−01 | 0.13 | 0.06 | 131 | 0 |
| SGK1 | 9 | 8.78E−01 | 0.20 | 0.06 | 132 | 0 |
| HTR2B | 66 | 8.79E−01 | 0.07 | 0.06 | 133 | 0 |
| PPARD | 9 | 8.87E−01 | 0.19 | 0.05 | 134 | 0 |
| CACNA1F | 13 | 9.06E−01 | 0.16 | 0.04 | 135 | 0 |
| FGFR3 | 12 | 9.19E−01 | 0.16 | 0.04 | 136 | 0 |
| CALM1 | 17 | 9.19E−01 | 0.13 | 0.04 | 137 | 0 |
| PARP1 | 16 | 9.27E−01 | 0.14 | 0.03 | 138 | 0 |
| KCNA5 | 11 | 9.33E−O1 | 0.16 | 0.03 | 139 | 0 |
| CES1 | 10 | 9.46E−01 | 0.17 | 0.02 | 140 | 0 |
| CYP17A1 | 9 | 9.55E−01 | 0.17 | 0.02 | 141 | 0 |
| HTR1F | 26 | 9.63E−01 | 0.10 | 0.02 | 142 | 0 |
| MMP9 | 12 | 9.73E−01 | 0.14 | 0.01 | 143 | 0 |
| CACNA1G | 17 | 9.81E−01 | 0.11 | 0.01 | 144 | 0 |
| ADRB3 | 34 | 9.82E−01 | 0.08 | 0.01 | 145 | 0 |
| FGFR2 | 13 | 9.84E−01 | 0.13 | 0.01 | 146 | 0 |
| HRH4 | 21 | 9.89E−01 | 0.10 | 0.00 | 147 | 0 |
| CA14 | 19 | 0.99 | 0.10 | 0.00 | 148 | 0 |
| KCNJ10 | 10 | 1.00 | −0.13 | 0.00 | 149 | 0 |
| CACNB3 | 9 | 0.98 | −0.15 | −0.01 | 150 | 0 |
| CACNB4 | 9 | 0.98 | −0.15 | −0.01 | 151 | 0 |
| GRIA3 | 13 | 0.98 | −0.13 | −0.01 | 152 | 0 |
| GRIA4 | 13 | 0.98 | −0.13 | −0.01 | 153 | 0 |
| SCN1A | 25 | 0.97 | −0.10 | −0.01 | 154 | 0 |
| SCN10A | 35 | 0.95 | −0.09 | −0.02 | 155 | 0 |
| GRIA2 | 22 | 0.95 | −0.11 | −0.02 | 156 | 0 |
| CHRNA3 | 13 | 0.94 | −0.15 | −0.03 | 157 | 0 |
| CACNB1 | 12 | 0.93 | −0.16 | −0.03 | 158 | 0 |
| SCN3A | 21 | 0.91 | −0.12 | −0.04 | 159 | 0 |
| CA7 | 12 | 0.91 | −0.16 | −0.04 | 160 | 0 |
| RPS6KB1 | 11 | 0.90 | −0.17 | −0.04 | 161 | 0 |
| HRH2 | 23 | 0.90 | −0.12 | −0.05 | 162 | 0 |
| SCN5A | 43 | 0.89 | −0.09 | −0.05 | 163 | 0 |
| PRKCA | 19 | 0.87 | −0.14 | −0.06 | 164 | 0 |
| CYP2C9 | 11 | 0.87 | −0.18 | −0.06 | 165 | 0 |
| GRIN2D | 23 | 0.87 | −0.12 | −0.06 | 166 | 0 |
| SCN9A | 21 | 0.86 | −0.13 | −0.07 | 167 | 0 |
| ADORA1 | 36 | 0.85 | −0.10 | −0.07 | 168 | 0 |
| HTR3A | 50 | 0.85 | −0.09 | −0.07 | 169 | 0 |
| ADORA3 | 26 | 0.84 | −0.12 | −0.08 | 170 | 0 |
| CACNA1I | 15 | 0.84 | −0.16 | −0.08 | 171 | 0 |
| ADRA1B | 69 | 0.83 | −0.08 | −0.08 | 172 | 0 |
| VDR | 17 | 0.82 | −0.15 | −0.09 | 173 | 0 |
| GRM1 | 9 | 0.80 | −0.21 | −0.09 | 174 | 0 |
| HSD11B1 | 12 | 0.80 | −0.19 | −0.09 | 175 | 0 |
| SCN2A | 19 | 0.79 | −0.15 | −0.10 | 176 | 0 |
| GSK3B | 29 | 0.79 | −0.12 | −0.10 | 177 | 0 |
| CA6 | 13 | 0.79 | −0.18 | −0.10 | 178 | 0 |
| GRIN2A | 32 | 0.77 | −0.12 | −0.12 | 179 | 0 |
| KCNJ8 | 11 | 0.75 | −0.20 | −0.12 | 180 | 0 |
| GRIK2 | 10 | 0.75 | −0.21 | −0.12 | 181 | 0 |
| CACNB2 | 15 | 0.74 | −0.18 | −0.13 | 182 | 0 |
| OPRM1 | 35 | 0.74 | −0.12 | −0.13 | 183 | 0 |
| GLRA1 | 18 | 0.74 | −0.16 | −0.13 | 184 | 0 |
| NOS2 | 19 | 0.74 | −0.16 | −0.13 | 185 | 0 |
| ADRA1A | 115 | 0.74 | −0.06 | −0.13 | 186 | 0 |
| HTR4 | 21 | 0.73 | −0.15 | −0.13 | 187 | 0 |
| TACR1 | 9 | 0.73 | −0.23 | −0.14 | 188 | 0 |
| SCN11A | 18 | 0.73 | −0.16 | −0.14 | 189 | 0 |
| SCN7A | 18 | 0.73 | −0.16 | −0.14 | 190 | 0 |
| SCN8A | 18 | 0.73 | −0.16 | −0.14 | 191 | 0 |
| CHRNB2 | 12 | 0.72 | −0.20 | −0.14 | 192 | 0 |
| TNF | 20 | 0.71 | −0.16 | −0.15 | 193 | 0 |
| CACNA1S | 17 | 0.70 | −0.17 | −0.15 | 194 | 0 |
| CNR2 | 12 | 0.70 | −0.20 | −0.15 | 195 | 0 |
| PPARG | 35 | 0.70 | −0.12 | −0.15 | 196 | 0 |
| CACNG1 | 11 | 0.70 | −0.21 | −0.15 | 197 | 0 |
| CACNA1H | 18 | 0.67 | −0.17 | −0.17 | 198 | 0 |
| CACNA2D2 | 10 | 0.67 | −0.23 | −0.18 | 199 | 0 |
| MAOB | 30 | 0.67 | −0.13 | −0.18 | 200 | 0 |
| ESR1 | 54 | 0.66 | −0.10 | −0.18 | 201 | 0 |
| CA4 | 24 | 0.66 | −0.15 | −0.18 | 202 | 0 |
| ALOX5 | 28 | 0.65 | −0.14 | −0.19 | 203 | 0 |
| CACNA1D | 18 | 0.65 | −0.17 | −0.19 | 204 | 0 |
| ADORA2B | 24 | 0.64 | −0.15 | −0.20 | 205 | 0 |
| MAOA | 31 | 0.64 | −0.13 | −0.20 | 206 | 0 |
| SLC18A2 | 9 | 0.63 | −0.25 | −0.20 | 207 | 0 |
| GRIN3B | 11 | 0.62 | −0.23 | −0.20 | 208 | 0 |
| KCNQ2 | 9 | 0.62 | −0.25 | −0.21 | 209 | 0 |
| PRKACA | 11 | 0.62 | −0.23 | −0.21 | 210 | 0 |
| GRIN1 | 42 | 0.62 | −0.12 | −0.21 | 211 | 0 |
| TRPV1 | 20 | 0.61 | −0.17 | −0.22 | 212 | 0 |
| ADRB1 | 67 | 0.60 | −0.09 | −0.22 | 213 | 0 |
| GABRA6 | 37 | 0.60 | −0.13 | −0.22 | 214 | 0 |
| ACHE | 34 | 0.60 | −0.13 | −0.23 | 215 | 0 |
| OPRD1 | 31 | 0.59 | −0.14 | −0.23 | 216 | 0 |
| CYSLTR1 | 9 | 0.58 | −0.26 | −0.24 | 217 | 0 |
| PTGER1 | 10 | 0.57 | −0.25 | −0.25 | 218 | 0 |
| SLC6A3 | 40 | 0.55 | −0.13 | −0.26 | 219 | 0 |
| CA12 | 28 | 0.55 | −0.15 | −0.26 | 220 | 0 |
| CHRNA7 | 23 | 0.52 | −0.17 | −0.29 | 221 | 0 |
| TSPO | 12 | 0.51 | −0.24 | −0.29 | 222 | 0 |
| GRIK1 | 10 | 0.49 | −0.26 | −0.31 | 223 | 0 |
| KCNJ11 | 17 | 0.48 | −0.20 | −0.32 | 224 | 0 |
| CA9 | 18 | 0.48 | −0.20 | −0.32 | 225 | 0 |
| AHR | 9 | 0.47 | −0.28 | −0.33 | 226 | 0 |
| ADRA1D | 59 | 0.47 | −0.11 | −0.33 | 227 | 0 |
| CA2 | 36 | 0.46 | −0.14 | −0.34 | 228 | 0 |
| ATP4A | 10 | 0.45 | −0.27 | −0.34 | 229 | 0 |
| GRIA1 | 24 | 0.44 | −0.18 | −0.35 | 230 | 0 |
| GABBR1 | 16 | 0.44 | −0.22 | −0.36 | 231 | 0 |
| GABRA1 | 60 | 0.43 | −0.11 | −0.37 | 232 | 0 |
| ABCC8 | 14 | 0.42 | −0.23 | −0.37 | 233 | 0 |
| TGFBR1 | 10 | 0.40 | −0.28 | −0.39 | 234 | 0 |
| CACNA1C | 30 | 0.40 | −0.16 | −0.40 | 235 | 0 |
| CCKBR | 9 | 0.40 | −0.30 | −0.40 | 236 | 0 |
| RRM1 | 11 | 0.40 | −0.27 | −0.40 | 237 | 0 |
| HTR3B | 18 | 0.39 | −0.21 | −0.41 | 238 | 0 |
| GABRA4 | 36 | 0.38 | −0.15 | −0.42 | 239 | 0 |
| KCNMA1 | 13 | 0.37 | −0.25 | −0.43 | 240 | 0 |
| SCN4A | 26 | 0.36 | −0.18 | −0.44 | 241 | 0 |
| ALK | 15 | 0.36 | −0.24 | −0.44 | 242 | 0 |
| OPRK1 | 39 | 0.34 | −0.15 | −0.47 | 243 | 0 |
| ADRB2 | 78 | 0.33 | −0.11 | −0.48 | 244 | 0 |
| PPARA | 18 | 0.33 | −0.22 | −0.48 | 245 | 0 |
| PTGDR2 | 9 | 0.32 | −0.32 | −0.50 | 246 | 0 |
| GRIN3A | 18 | 0.31 | −0.23 | −0.51 | 247 | 0 |
| GLRA2 | 10 | 0.31 | −0.31 | −0.51 | 248 | 0 |
| CA1 | 39 | 0.28 | −0.16 | −0.55 | 249 | 0 |
| RET | 16 | 0.28 | −0.25 | −0.56 | 250 | 0 |
| NOS1 | 9 | 0.28 | −0.33 | −0.56 | 251 | 0 |
| AGTR1 | 16 | 0.25 | −0.26 | −0.60 | 252 | 0 |
| LCK | 18 | 0.25 | −0.24 | −0.61 | 253 | 0 |
| ADRA2B | 65 | 0.22 | −0.13 | −0.65 | 254 | 0 |
| CACNA2D1 | 17 | 0.21 | −0.26 | −0.68 | 255 | 0 |
| ABL1 | 17 | 0.20 | −0.26 | −0.69 | 256 | 0 |
| AKR1B1 | 12 | 0.20 | −0.31 | −0.70 | 257 | 0 |
| EDNRA | 11 | 0.19 | −0.33 | −0.71 | 258 | 0 |
| MTNR1A | 9 | 0.19 | −0.36 | −0.73 | 259 | 0 |
| MTNR1B | 9 | 0.19 | −0.36 | −0.73 | 260 | 0 |
| ADRA2C | 64 | 0.18 | −0.14 | −0.75 | 261 | 0 |
| DPP4 | 12 | 0.17 | −0.32 | −0.76 | 262 | 0 |
| NOS3 | 15 | 0.17 | −0.29 | −0.77 | 263 | 0 |
| PDE1A | 9 | 0.17 | −0.37 | −0.78 | 264 | 0 |
| RRM2 | 9 | 0.17 | −0.37 | −0.78 | 265 | 0 |
| ADORA2A | 31 | 0.17 | −0.20 | −0.78 | 266 | 0 |
| PDGFRA | 22 | 0.14 | −0.25 | −0.86 | 267 | 0 |
| PDE5A | 20 | 0.14 | −0.26 | −0.87 | 268 | 0 |
| CDK5 | 13 | 0.13 | −0.32 | −0.88 | 269 | 0 |
| GLRB | 16 | 0.13 | −0.29 | −0.89 | 270 | 0 |
| ACE | 23 | 0.13 | −0.25 | −0.90 | 271 | 0 |
| ERBB2 | 27 | 0.12 | −0.23 | −0.91 | 272 | 0 |
| PDE3A | 20 | 0.10 | −0.27 | −1.00 | 273 | 0 |
| ADRA2A | 83 | 0.06 | −0.15 | −1.19 | 274 | 0 |
| ESR2 | 34 | 0.06 | −0.23 | −1.23 | 275 | 0 |
| CACNA1B | 13 | 0.05 | −0.38 | −1.29 | 276 | 0 |
| PTPN1 | 11 | 0.05 | −0.41 | −1.32 | 277 | 0 |
| CFTR | 14 | 0.05 | −0.37 | −1.33 | 278 | 0 |
| MMP2 | 12 | 0.05 | −0.40 | −1.34 | 279 | 0 |
| HMGCR | 12 | 0.04 | −0.40 | −1.36 | 280 | 0 |
| PDE4B | 22 | 0.03 | −0.31 | −1.50 | 281 | 0 |
| KCNQ1 | 11 | 0.03 | −0.44 | −1.55 | 282 | 0 |
| HSP90AA1 | 15 | 0.02 | −0.38 | −1.61 | 283 | 0 |
| PTGS1 | 80 | 0.02 | −0.17 | −1.71 | 284 | 0 |
| CDK1 | 22 | 0.02 | −0.33 | −1.72 | 285 | 0 |
| PNP | 9 | 0.01 | −0.53 | −1.87 | 286 | 0 |
| MAP2K2 | 9 | 0.01 | −0.53 | −1.89 | 287 | 0 |
| PDE4C | 15 | 0.01 | −0.44 | −2.17 | 288 | 0 |
| SRC | 17 | 0.01 | −0.41 | −2.20 | 289 | 0 |
| PDE4A | 24 | 0.00 | −0.36 | −2.42 | 290 | 0 |
| PDE4D | 19 | 0.00 | −0.41 | −2.44 | 291 | 0 |
| EGFR | 54 | 0.00 | −0.25 | −2.53 | 292 | 0 |
| PTGS2 | 90 | 0.00 | −0.19 | −2.55 | 293 | 0 |
| MAP2K1 | 18 | 0.00 | −0.45 | −2.83 | 294 | 0 |

### XI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Pan-Cancer Convergence to a Small Cell Neuroendocrine Phenotype that Shares Susceptibilities with Hematological Malignancies

Small cell neuroendocrine (SCN) cancers are an aggressive cancer subtype. In prostate and lung adenocarcinoma, transdifferentiation towards a SCN phenotype is a resistance route in response to targeted therapies. Here, the inventors identified pan-tissue convergence to a SCN state characterized by shared genome-wide patterns of expression, methylation, and copy number alteration. The inventors find that the SCN molecular phenotype is more widespread across various epithelial cancers than previously realized, with these additional cases associated with poor prognosis. More broadly, non-SCN metastases have higher expression of SCN-associated transcription factors than non-SCN primary tumors. Experimental drug sensitivity and gene dependency screens demonstrate that these convergent SCN cancers have shared vulnerabilities. These common vulnerabilities are found across unannotated SCN-like epithelial cases, pediatric small round blue cell tumors, and unexpectedly in hematological malignancies. The SCN convergent phenotype and common sensitivity profiles with hematological cancers can guide treatment options beyond the limitations of tissue-specific targeted therapies.

Small cell neuroendocrine (SCN) cancers are a histologically similar and highly aggressive cancer subtype that appears across tissue types. No effective treatment modalities are available. Non-SCN epithelial cancers have been shown to transdifferentiate to SCN cancers in response to targeted therapy. This has important consequences in that SCN cancers, once considered rare, may become increasingly common with the emergence of resistance cases from targeted therapies. Here the invenotrs define molecular signatures for SCN cancers, and the inventors find that SCN cancers share similar drug and RNAi vulnerabilities with blood cancers. The inventors' results guide the detection of SCN-like cases in the clinic, and support the exploration of treatments for SCN cancers that mimic treatments for blood cancers.

A. Introduction

Neuroendocrine cells are found in numerous tissues, but the cell of origin of SCNCs across tissues is unclear. Small cell lung cancer (SCLC) can arise de novo, possibly from a cell of neuroendocrine origin, as seen in mouse models or in transformation experiments performed in distinct lung cell types (Park et al., 2011; Sutherland et al., 2011). A second proposed mechanism for SCLC origin is through transdifferentiation from another non-neuroendocrine cell lineage, which has been observed in patient tumors, and studied in mouse in vivo experiments (Niederst et al., 2015; Yang et al., 2018). Across multiple tissues types, adenocarcinomas have been observed to escape targeted therapy through evolution to a histologically similar SCN phenotype, highlighting the need to characterize and develop therapies for this aggressive cancer outcome. There are currently no effective therapies for SCNCs. SCNC is considered a systemic disease and typically leads to early metastases. Etoposide or platinum-based chemotherapies are the primary first-line treatment modalities (Klimstra et al., 2015; Nadal et al., 2014). These treatments are only transiently effective, and 5-year survival rates for SCN lung and prostate cancers are less than 20% (Alanee et al., 2015). Here the inventors sought to provide a molecular and functional underpinning for the observed pathology-based similarities between SCN cancers arising in multiple tissue types. In an unsupervised fashion, the data led to an unanticipated signature and vulnerability similarity between SCN cancers and hematopoietic malignancies.

B. Results

1. Small Cell Neuroendocrine Cancers Across Tissues Converge on a Common Molecular Phenotype

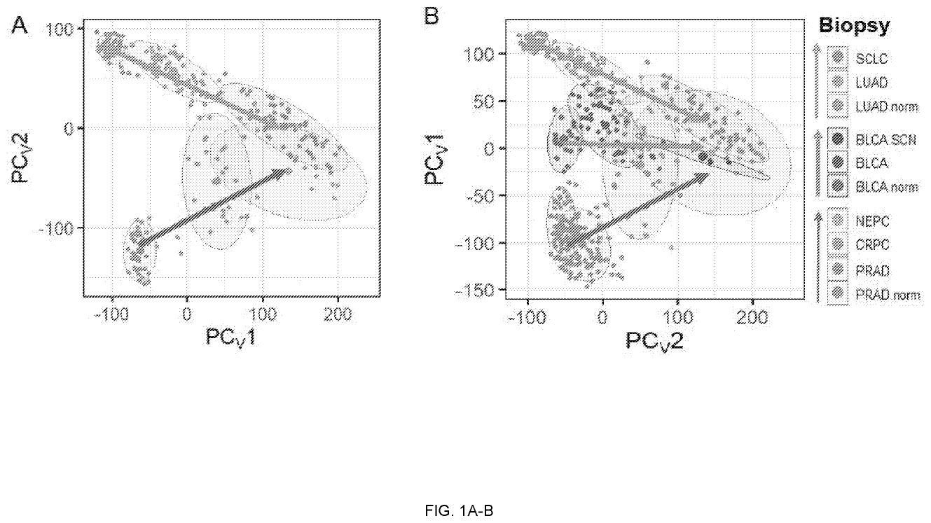

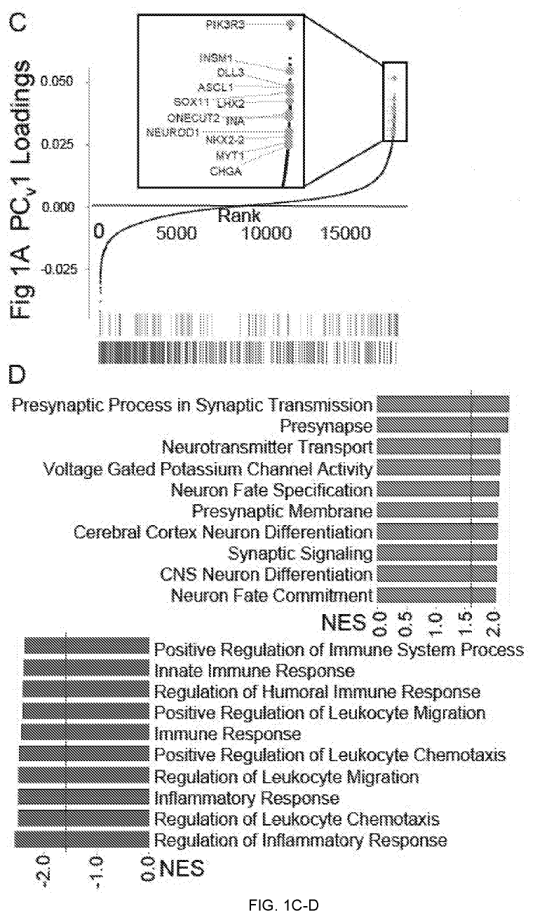

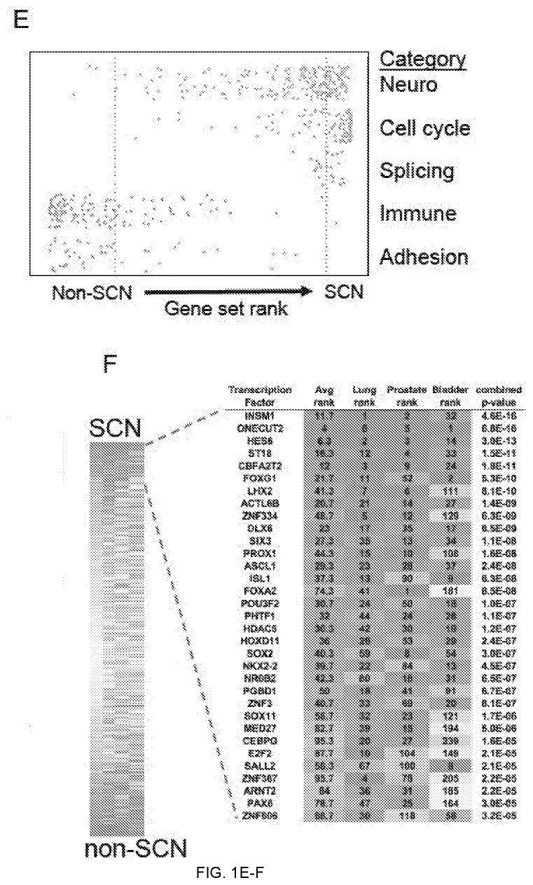

With the advent of global-omic profiling of SCN cancers (Beltran et al., 2016; George et al., 2015; Robertson et al., 2018), the inventors aimed to comprehensively define and explore the molecular similarities of SCN tumors across various tissues. An unsupervised principal component analysis (PCA) performed on RNA-seq datasets of tumor biopsies of lung adenocarcinoma (LUAD), castration-resistant prostate adenocarcinoma (CRPC-adeno), small cell lung cancer (SCLC), and castration-resistant neuroendocrine prostate cancer (NEPC), along with both normal lung and prostate tissues, revealed a strongly convergent expression signature (FIG. 1A), as previously reported in comparisons of normal tissue and SCN tumors (Park et al., 2018). The inventors further find that as tumor cell states progress along a transdifferentiation trajectory from adenocarcinoma to SCNC (arrows), the tumors become increasingly independent of tissue of origin, and converge to a shared set of features reflecting a common SCN state.

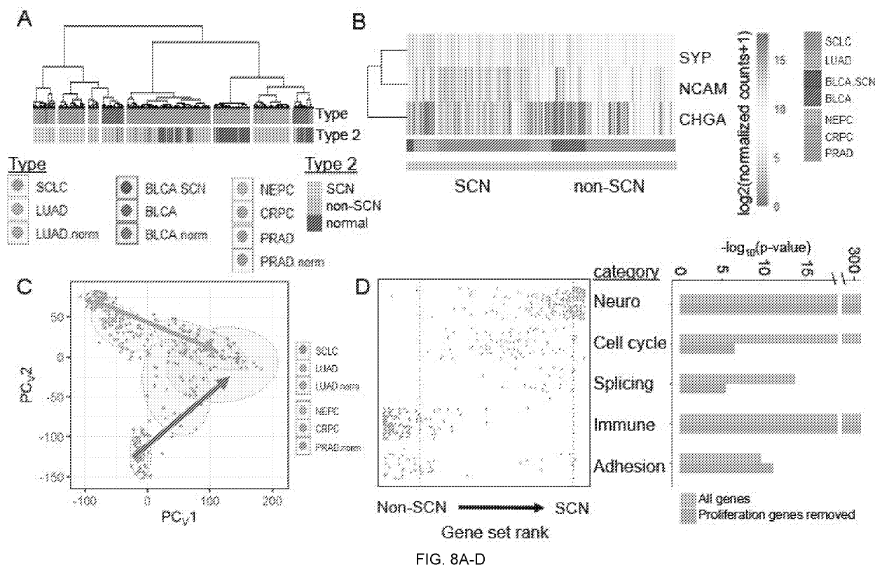

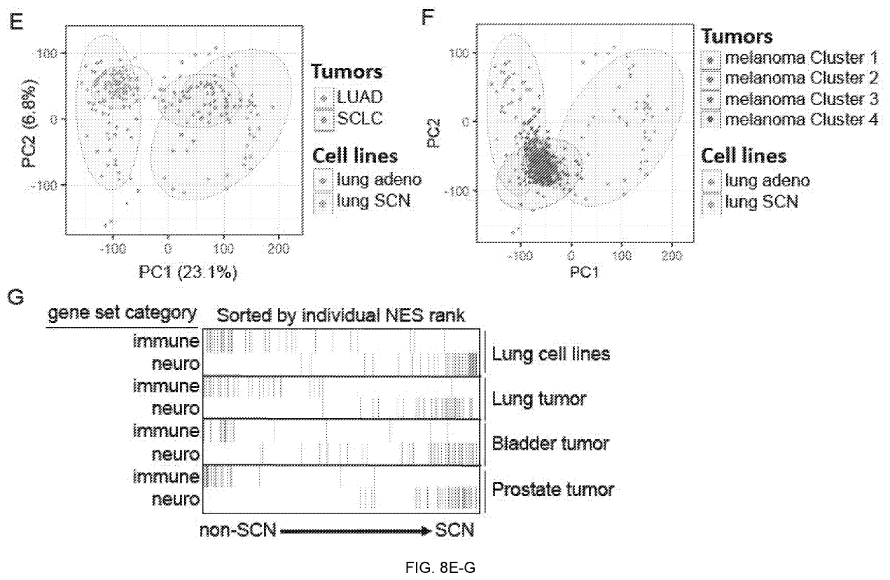

Four samples of bladder cancer (BLCA) in the TCGA, recently reclassified as having small cell neuroendocrine features (Robertson et al., 2018), also converge with the lung and prostate SCN cases when included in the PCA analysis (FIG. 1B), with the PCA recapitulating a developmental landscape for the three tissues. Hierarchical clustering of the samples supported that SCNCs of these three epithelial tissues were more similar to each other than to their non-SCNC counterparts (FIG. 8A). The genes that most strongly contribute to the SCN signature in the pan-tissue analysis are enriched for known small cell neuroendocrine-associated genes such as CHGA and INSM1, and genes related to neural transcriptional programs such as ASCL1, NEUROD1, SEZ6, INA, and NKX2-2 (FIG. 1C). It is important to note that any one cancer incidence may contain only a subset of these markers (FIG. 8B) and hence be missed by traditional classification schemes based on only a few markers (Oberg et al., 2015). In sum, unsupervised analyses revealed that SCN cancers of different tissues are more similar to each other than are adenocarcinomas of different tissues.

To further investigate the function of genes differentially expressed along the normal to SCNC convergent trajectory, the inventors applied enrichment analysis to the ranked list of genes. Enriched in the SCN state were gene sets related to neuron development and neuronal function, as well as splicing and cell cycle, while the de-enriched gene sets included those related to adhesion, and to immune response and inflammation (hereafter referred to as immune gene sets) (FIG. 1D-E). To determine the contribution of proliferation genes on convergence, the inventors removed proliferation and proliferation-associated genes, and found that the convergence of SCNCs across tissues was maintained (FIG. 8C). Enrichment analysis on ranked genes from the proliferation gene-removed PCA confirmed the loss of enrichment of cell cycle gene sets, and the maintenance of enrichment of neuronal gene sets (FIG. 8D).

2. A Common Transcription Factor Network is Reflected in Small Cell Neuroendocrine Cancer Profiles Across Tissues

The inventors built a data-guided transcription network across lung, prostate, and bladder SCN and non-SCN tumor datasets using ARACNe to uncover the transcription factor network defining SCNCs. The inventors employed the Virtual Inference of Protein-activity by Enriched Regulon (VIPER) algorithm to infer protein activity from gene expression data. This analysis revealed a remarkable similarity in data-driven inferred transcription factor activity across lung, prostate, and bladder SCNCs (FIG. 1F). The transcription factors identified included multiple factors central to neural development and brain patterning, such as LHX2, HES6, PROX1, PAX6, MYT1, and NKX2-2 (data not shown). These genes highlight the influence of neuronal gene programs and transcription factors in multiple tissues in the development of a SCNC identity.

Gene expression-based PCA defined on lung adeno and lung SCN cell lines faithfully predicted SCN cases of lung tumors (FIG. 8E), but not other transitions such as melanoma dedifferentiation (Tsoi et al., 2018) (FIG. 8F), supporting cell lines as an informative model for the interrogation of the SCN and non-SCN dichotomy. Neuronal gene sets were enriched in SCN tumors compared to their tissue-respective adenocarcinomas, while immune gene sets were de-enriched. Interestingly, the same immune gene sets were de-enriched in small cell lung cancer cell lines, which do not contain an immune cell subpopulation as tumor biopsies do, indicating that the reduction of canonical immune and inflammation mediators in the SCN state is in part cancer cell-intrinsic (FIG. 8G).

3. The Shared Small Cell Neuroendocrine Gene Expression Signature has an Epigenomic Basis

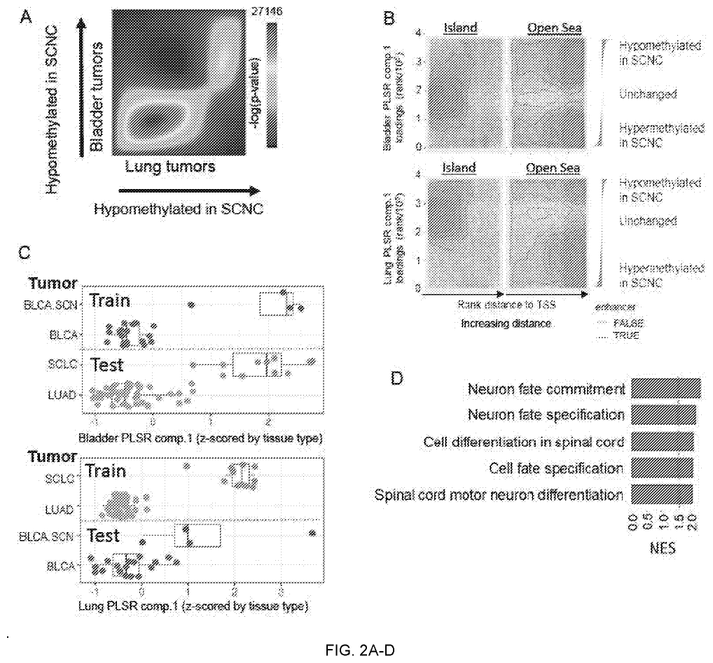

As epigenetic mechanisms regulate gene expression and differentiation, the inventors investigated changes in DNA methylation patterns that accompany the SCN transition. Signature overlap analysis using rank-rank hypergeometric overlap (RRHO) on PLSR loadings-based signatures created individually from either lung or bladder cancers showed that the methylation signatures were highly similar (FIG. 2A) (Plaisier et al., 2010). To characterize the methylation sites distinguishing SCNCs from their adenocarcinoma counterparts, the inventors plotted methylation sites by their rank in the PLSR loadings (prostate was not included here due to much fewer sites measured by the corresponding microarray platform). This analysis revealed that sites within open-sea regions (typically distant from the transcriptional start site (TSS)), rather than CG island regions (TSS proximal), are important in contribution to the SCN to non-SCN distinction (1-sided KS-test p value<2.2×10-16) (FIG. 2B). Distinct from the canonical role of DNA methylation in regulating transcription at gene promoters, these distal methylation changes that delineate the epigenetic differences between SCN and non-SCN cancers are consistent with enhancer-based regulation of gene expression programs (Sur and Taipale, 2016).

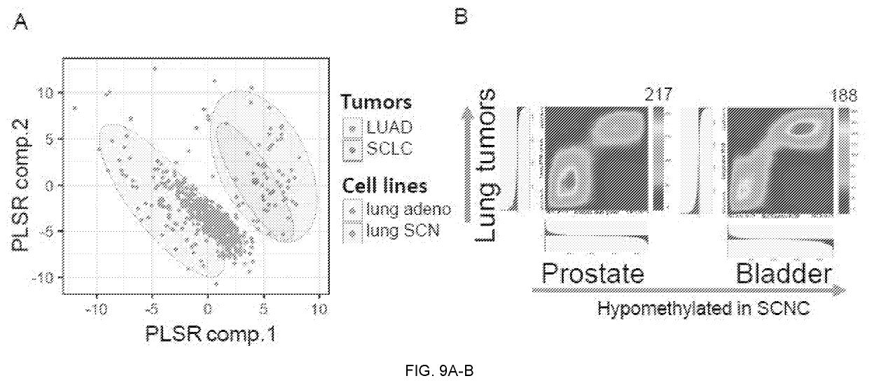

The inventors next investigated whether the methylation sites distinguishing lung SCN from non-SCN tumors could separate the same groupings in bladder cancer, or vice versa. When the inventors projected lung cancer samples to a PLSR methylation signature based on the bladder non-SCN-SCN dichotomy, they observed that these sites on average distinguished SCLC from LUAD (FIG. 2C). In the reverse direction, when BLCA samples were projected onto a PLSR-based framework trained on the lung tumor biopsies, not all the BLCA SCN samples were completely distinct from the BLCA non-SCN samples, but on average had methylation patterns more similar to lung SCLC, highlighting the concordance of non-SCN versus SCN methylation profiles across tissues (FIG. 2C). To gain insight into the genes being regulated by methylation changes in SCNCs across multiple tissues, the inventors co-analyzed the subset of sites measured in all datasets (lung, bladder, and prostate). Projection of gene-summarized methylation values of lung tumor samples onto a PLSR analysis of lung cell lines confirmed concordance of methylation patterns between tumors and cell lines (FIG. 9A). Pairwise RRHO analyses supported the methylation-based concordance of SCN tumors from all three tissues (FIG. 9B). Gene-based summarization of methylation sites revealed that the top differentially enriched gene sets that appeared across lung, prostate, and bladder tissues were strongly related to neuronal development (FIG. 2D). Taken together, the methylation analysis supports that the pan-tissue convergent similarity in SCNCs is functionally maintained across their methylomes.

4. Selection for Small Cell Neuroendocrine-Associated DNA Copy Number Alteration Signatures Across Tissue Types

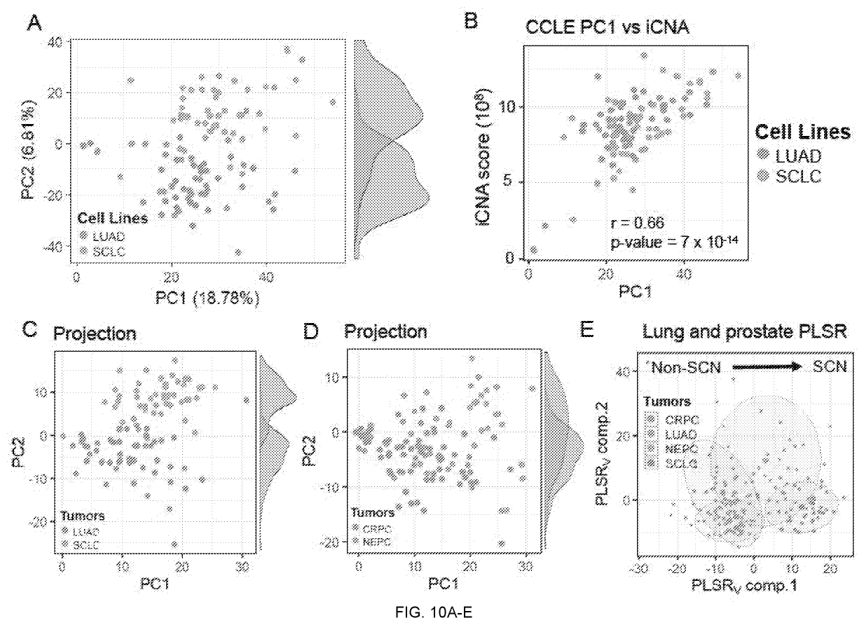

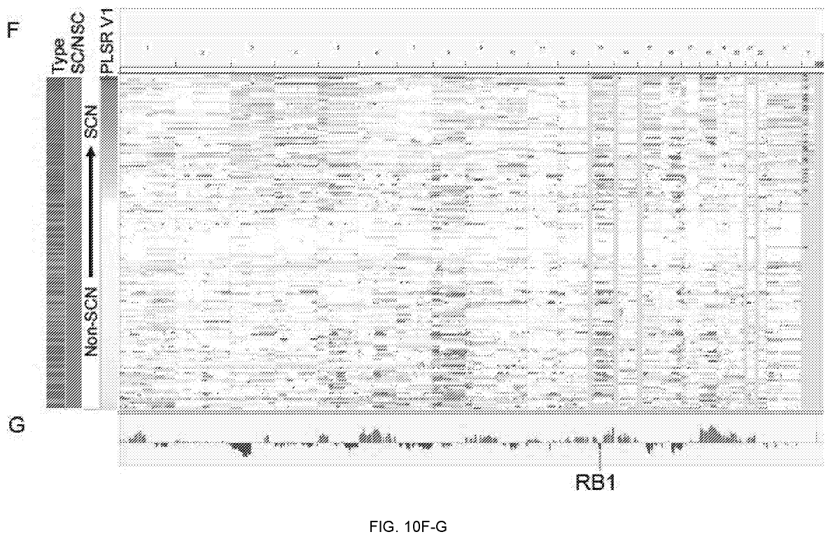

Recurrent genomic scars in cancer are reflective of current and past selective forces that confer fitness advantages through copy number changes in distinct genomic regions (Graham et al., 2017). PCA of DNA copy number patterns in lung cell lines in the Cancer Cell Line Encyclopedia (CCLE) showed clear differences in the copy number amplification and deletion patterns between SCN and non-SCN lines along the second principal component (FIG. 10A), with the first principal component describing the overall degree of aneuploidy of each tumor sample (FIG. 10B). The inventors projected patient tumor samples onto this PCA framework and found that the CNA patterns in the SCN lung cancer (SCLC) cell lines were reflected in both SCLC (FIG. 10C) and NEPC (FIG. 10D) patient tumors. To determine consistently amplified or deleted regions in SCN cases of the two tissue types, the inventors performed PLSR on lung and prostate samples together (FIG. 10E-F) or independently (FIG. 10G), regressing on a binary phenotype of SCN or non-SCN. In the independent PLSR analyses results, the inventors examined the genomic loci loadings for consistencies (FIG. 10G). This consistency analysis demonstrated that SCNCs shared particular amplifications and deletions, such as 1p amplification and 3p deletion. As a positive control, the inventors noted that RB1 was included in the consistent deletion region on chromosome 13. Overall, the shared CNA patterns support that common selective forces act on small cell variants in both lung and prostate cancers. Furthermore, these specific CNA changes are seen in both de novo (lung) and treatment-induced transdifferentiation (prostate) cases of SCN cancers, supporting that the selective pressure of different tumor development pathways can similarly shape the copy number landscape of SCNCs.

5. A Pan-Cancer Predictor of Small Cell Neuroendocrine Cancers Reveals Unannotated SCN Cases

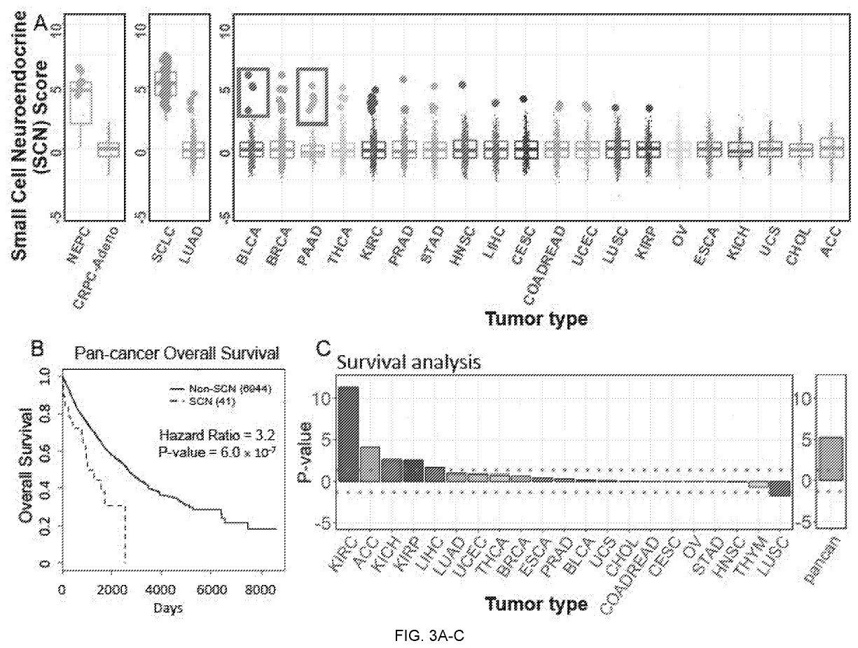

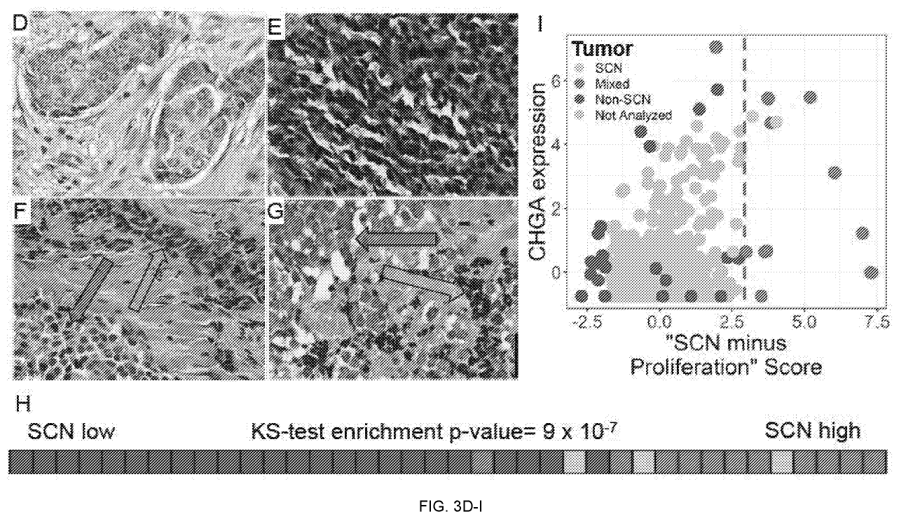

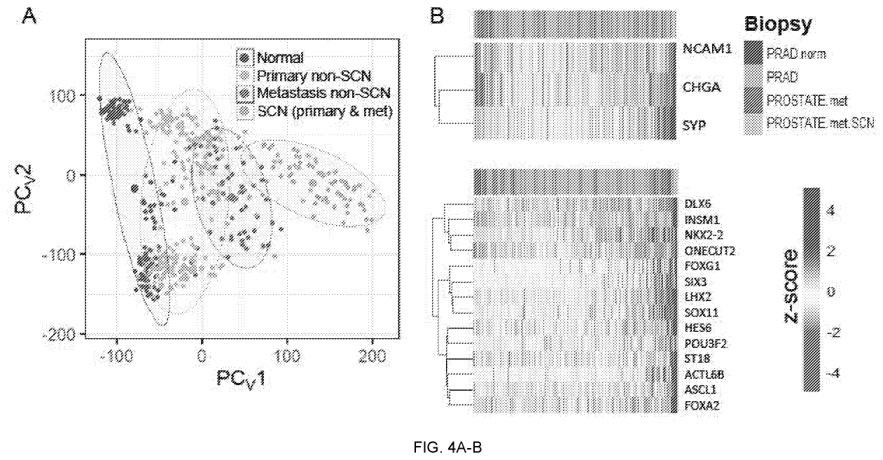

Clinical classification of SCNCs relies on histology and pathology defined by a few sets of morphological and molecular features. To uncover cases missed by these traditional approaches the inventors applied global omics profile-based scoring in previously analyzed datasets of purported non-SCN tumors (data not shown). Namely, the inventors used the gene expression-defined PCA SCN convergence framework, which provides a weighting for each gene (FIG. 1A), to predict SCN phenotypes across approximately 7000 primary TCGA tumors from 21 different epithelial tumor types (FIG. 3A). Boxplots of the first component in the prediction model reveal a low frequency of primary tumor cases that contain a SCN component (˜1%). Known SCN cases previously annotated in bladder as well as non-SCN primitive neuroectodermal tumors (PNET) of the pancreas were predicted using the inventors' classifier (red boxes FIG. 3A). Except for the low grade pancreatic PNETs, these samples were high grade tumors (data not shown), consistent with reports in the literature for the grades of pathology-defined SCN samples (Oronsky et al., 2017; Watson et al., 2018). Of note, there were many high-grade samples that are not SCN-like. Thus, SCN high-grade tumors may need to be treated differently in the clinic compared to other (non-SCN) high-grade tumors.

As SCN cases are often highly aggressive, the inventors compared the transcriptome-based predicted SCN (referred to as SCN-like) to non-SCN cases, controlling for tumor type, and found a significant decrease in patient overall survival in the SCN-like cases (excluding the indolent primitive neuroectodermal tumor cases found in pancreatic adenocarcinoma) (FIG. 3B). Association of the SCN phenotype with poorer overall survival was robust to changes in the SCN-score threshold used (data not shown), supporting that the SCN phenotype exists along a spectrum. This is confirmed, in both individual and pan-cancer survival analysis based on the continuous SCN score, in the majority of epithelial cancers (FIG. 3C). The traditional classification is that 30% of a tumor needs to be neuroendocrine for a cancer to be called neuroendocrine histologically, creating a dichotomous classification (Oronsky et al., 2017). The inventors' model places each cancer sample along a small cell neuroendocrine spectrum, and supports that cancers with higher levels of SCN features have increasingly poorer outcomes, even those with SCN features in the range that they are not overtly annotated as SCN by pathology analysis.

The inventors next investigated the histological features of the tumors most strongly predicted to be SCN-like based on the gene expression-derived SCN signature score. Notably, a number of TCGA cases received SCN scores greater than 3 standard deviations above the mean, although almost all were not diagnosed as neuroendocrine carcinoma based on the original pathology reports. To confirm the computational prediction, the inventors chose high signature-score outlier samples based on proliferation-removed SCN score across multiple tissue types (CESC, COADREAD, ESCA, HNSC, LUAD, LUSC, PRAD, STAD, THCA) and a pathologist analyzed the corresponding hematoxylin and eosin (H&E)-stained histology slides. Fourteen of 16 cases were confirmed by the pathology analysis to have SCN or neuroendocrine features. Cases with no SCN region identified may be explained by tumors with focal SCN, and pathology calls being performed on tissue not adjacent to the tissue used for sequencing (Table S5; web resource online at systems.crump.ucla.edu/scn/pathology_images.html).

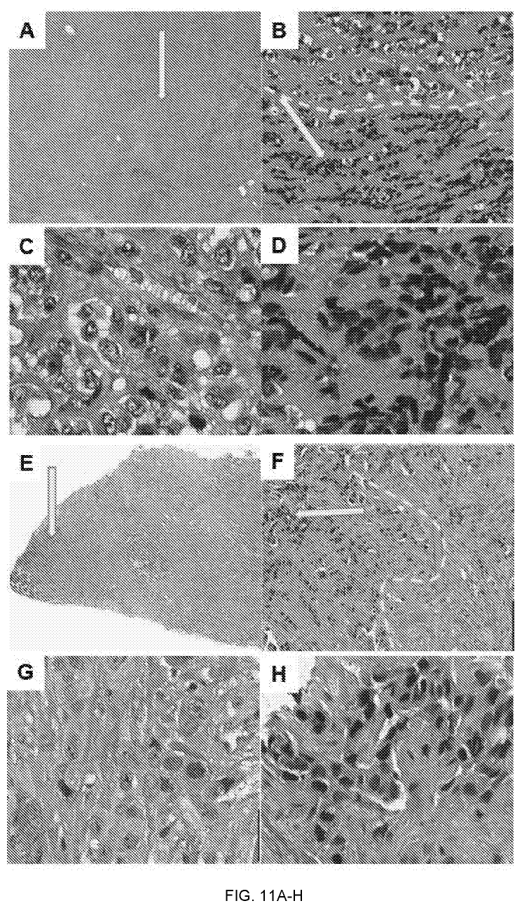

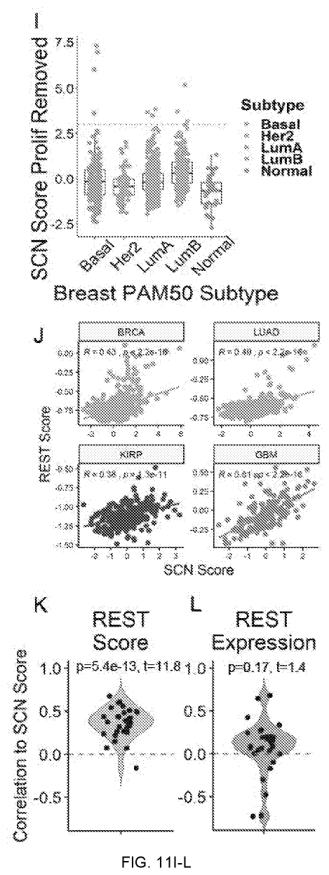

Because SCN breast cancers are considered rare, with a reported incidence of 0.1% (Wang et al., 2014), the inventors further investigated whether the predicted SCN-like BRCA cases in the TCGA indeed contained SCN features, which would suggest an under-diagnosis of breast cancer cases with SCN features. In this analysis, the inventors ranked samples by their proliferation-removed SCN score. A pathologist thoroughly examined a select number of H&E stained slides of TCGA breast tumor cases covering a full range of SCN scores (FIG. 3D-G; FIG. 11A-H; Table S5; web resource: https://systems.crump.ucla.edu/scn/pathology_images.html). The SCN score was statistically predictive of samples with pathology-based SCN features (p value=9.1×10-7; Kolmogorov-Smirnov enrichment test) (FIG. 3H). Thus, the pan-tissue pathology analysis validated the transcriptome-based SCN signature score as a predictor of tumors with SCN morphology features.

In the pathology analysis, high SCN signature-score BRCA samples were typically called either SCN-positive, or more often as having mixed histology (cells with SCN features mixed with a non-SCN breast tumor subtype, most frequently invasive ductal carcinoma). Three of 4 breast cancer subtypes (Basal, Luminal B, Luminal A) displayed regions with SCN pathology, usually in cases with accompanying genetic dysregulation of TP53 and RB1, suggesting a subset of cases for which closer pathologic interrogation will be beneficial to uncover the often focal regions of SCN morphology (FIG. 11I, Table S5). The SCN score-high BRCA samples did not uniformly express the traditional SCN markers of CHGA, SYP, and NCAM1 (FIG. 3I, Table S5). This finding reinforces the appreciation that heterogeneity in expression precludes the use of only a small set of markers in the clinical identification of aggressive SCN signature-positive tumor cases and their accompanying poorer prognosis.

The negative regulator of neural gene expression REST/NRSF functions as a transcriptional repressor. Loss of REST transcriptional suppression activity has been linked to promoting the SCN phenotype in NEPC (Zhang et al., 2015). In SCLC, REST induction by Notch in part drives inhibition of neuroendocrine differentiation in a subset of tumor cells, thus helping enforce tumor heterogeneity (Lim et al., 2017). The inventors' analysis supports that REST activity regulates the SCN phenotype in a pan-cancer manner (FIG. 11J-L). Namely, the expression of REST repressional target genes is positively correlated to SCN score across many epithelial and other tumor types, consistent with loss of REST-mediated suppression with increasing SCN score (FIG. 11K, p value=5.4×10-13, one-way t-test). Overall the SCN score and pathology analyses above validate that tumors with SCN-like features can be missed by standard pathology examination, and thus may be present more commonly than currently appreciated in cancer types in which SCNCs are considered rare, such as in breast cancer.

6. Mutations Associated with the Pan-Tissue Small Cell Neuroendocrine Phenotype

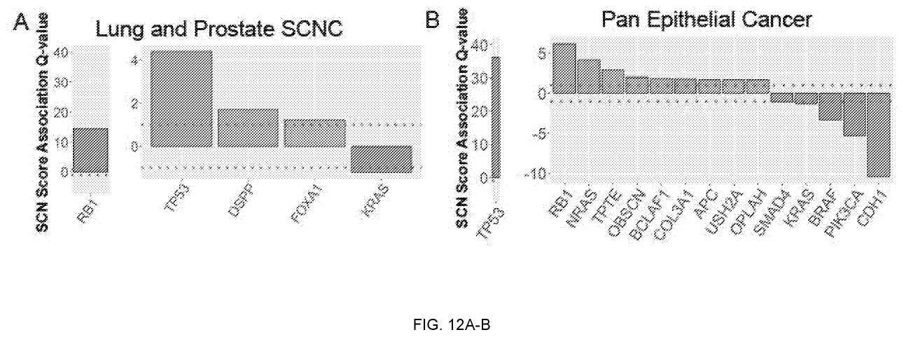

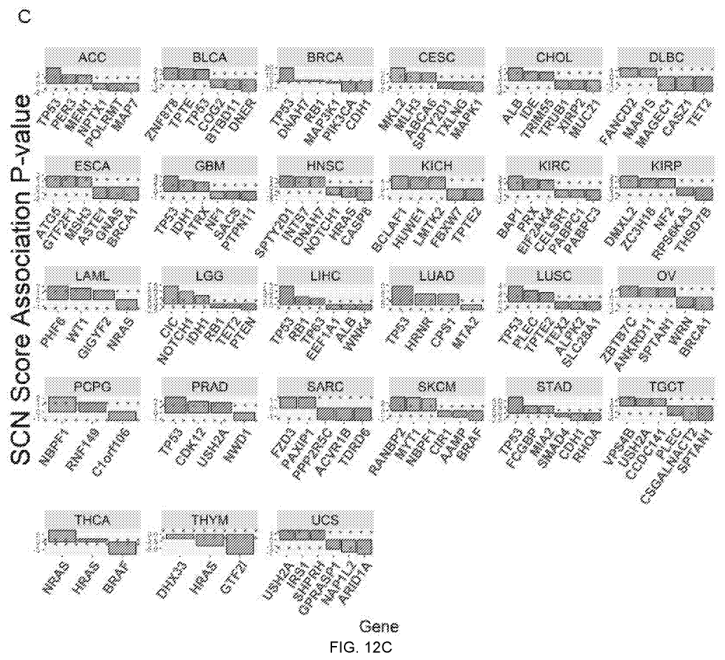

Mutations in tumor suppressor genes TP53 and RB1 are known to be highly associated with lung and prostate SCNCs (Beltran et al., 2016; George et al., 2015), and TP53 and RB1 loss have been shown to contribute to the formation of SCN-like histology and the transdifferentiation of adenocarcinomas to NEPC in vivo (Ku et al., 2017). By interrogating the spectrum of primary samples, the inventors sought to identify additional mutations associated with SCNCs in a pan-tissue context. The inventors applied a logistic regression model to fit mutation data in the TCGA dataset to SCN score, controlling for cancer type. The inventors first investigated the known cases of SCNC in lung and prostate tissues, which confirmed substantial and statistically significant TP53 and RB1 mutations as well as significant association with mutation of FOXA1, whose wild-type expression inhibits transition to NEPC (Kim et al., 2017) (FIG. 12A). In non-SCN epithelial cancers the inventors found that TP53 and RB1 mutations are associated with higher SCN score (FIG. 12B). Additionally the inventors uncovered SCN-associated mutations in NRAS (neuroblastoma-RAS) (FIG. 12B) and genes such as OBSCN and BCLAF1 that have been previously associated with tissue specific SCNCs (Cho et al., 2016; Rudin et al., 2012). In contrast, KRAS mutations were enriched in cases on the non-SCN side of the spectrum. Even in cancer types that are already highly neuronal such as glioblastoma (GBM), TP53 mutations were still associated with higher SCN score (FIG. 12C). This result aligns with published findings that the proneural subtype of GBM has a higher preponderance of TP53 and IDH1 mutations, while the mesenchymal subtype is enriched for NF1 mutations (Verhaak et al., 2010). Taken together, these results point to mutations beyond TP53 and RB1 that are common to SCN cancers across tissue types, and supports their mutational contribution to the development of SCN phenotypes.

7. Metastatic Non-SCN Tumors Express the SCN Signature Profile More Strongly than Primary Non-SCN Tumors

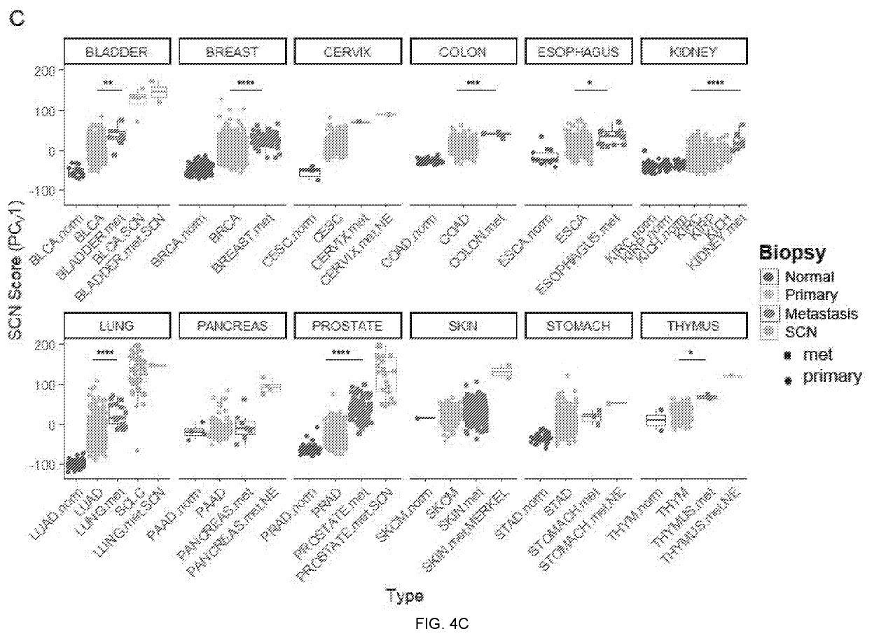

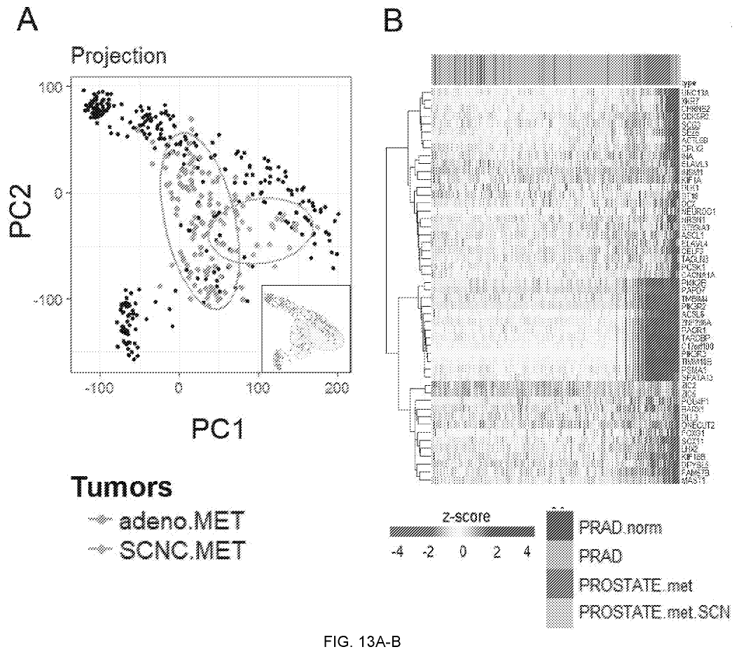

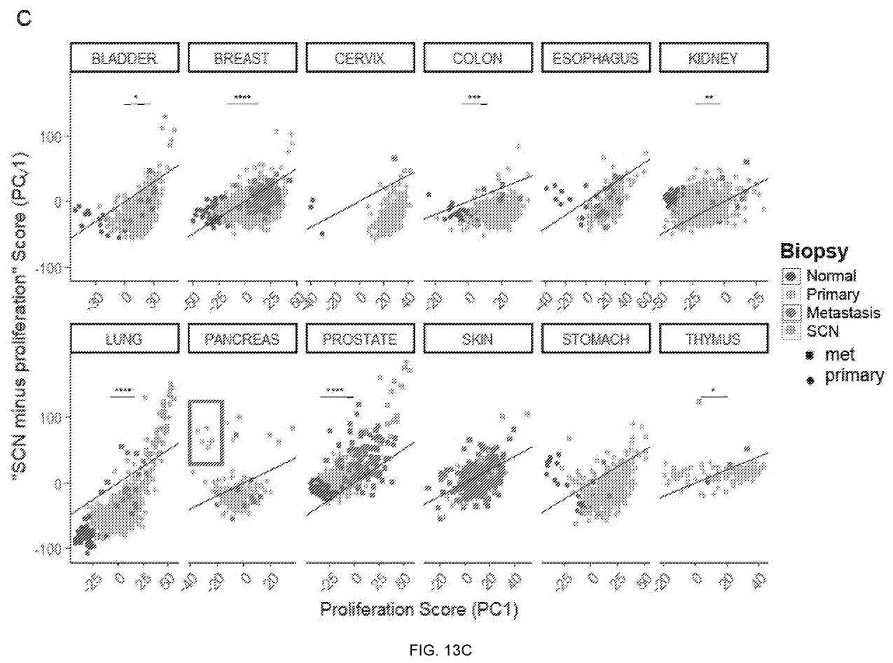

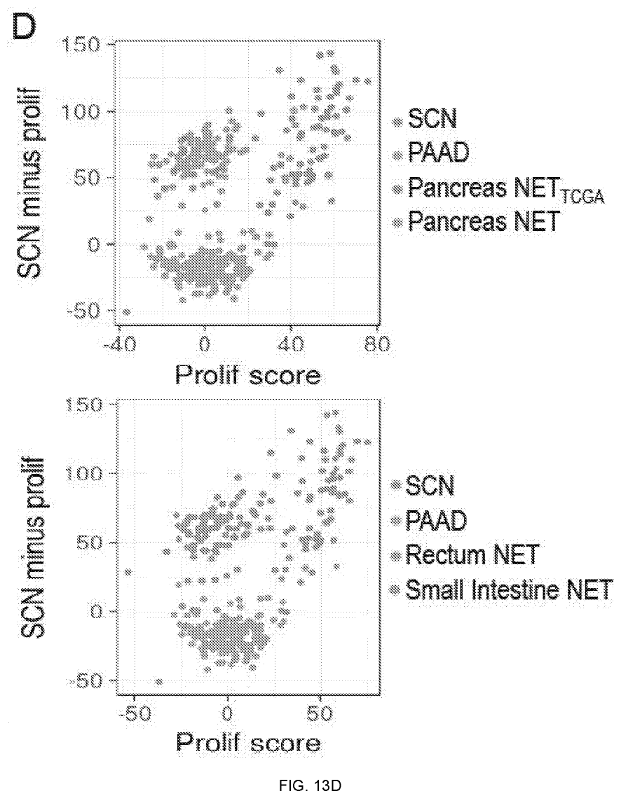

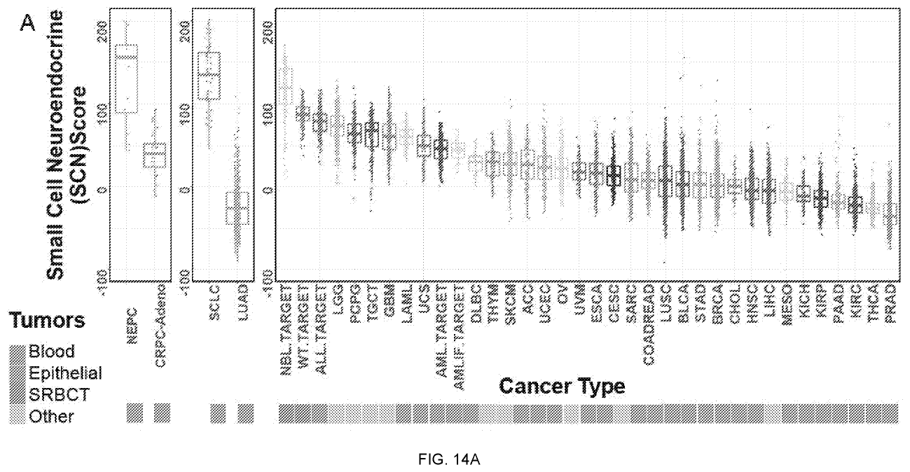

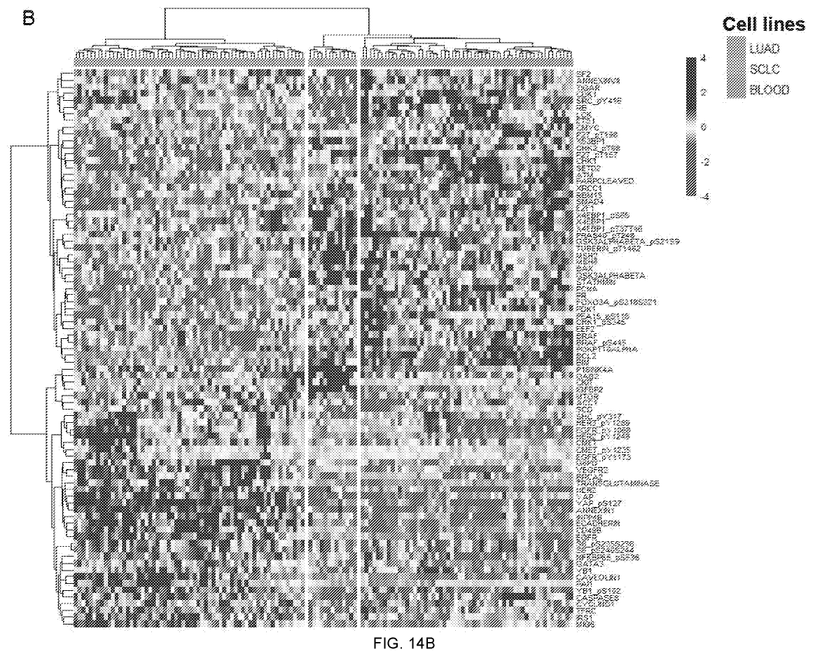

Given that SCNCs can arise from epithelial tissues, the inventors investigated the extent of the SCN signature in metastatic adenocarcinomas in comparison to primary adenocarcinomas. For lung, prostate, and bladder tissues, the inventors found that the expression profiles of metastatic adenocarcinoma samples were on average more similar to SCNCs than were their respective primary adenocarcinomas (FIG. 4A). Metastatic adenocarcinoma samples typically do not express canonical SCN markers, but express increasing levels of other SCN-associated genes and key transcription factors (FIG. 4B). To investigate this finding at a pan-tissue scale, the inventors analyzed a published dataset of metastases across many different tissues, which included both adenocarcinoma and SCNC metastases (Robinson et al., 2017). To determine the SCN signature score of metastatic adenocarcinoma and SCNC samples from all tissues in this metastasis dataset, they were projected onto the SCN framework of FIG. 1A (FIG. 13A). Plotting the expression levels of the top 50 genes of the SCN signature in prostate cancers visually demonstrated the increased similarity of prostate metastatic adenocarcinomas to prostate SCN cancers (FIG. 13B). The inventors then annotated metastatic samples by their site of origin and scored normal, primary, metastatic, and SCNCs by their position on the SCN spectrum (FIG. 4C). This analysis revealed that metastatic non-SCN samples tended to have SCN-score distributions significantly shifted upwards on the SCN spectrum in relation to their respective primary non-SCN samples, in multiple different tissues.

Because the SCN score contains both neuronal and proliferation components (FIG. 1E), the inventors sought to deconvolute these components and define the contribution of each in the primary, metastatic, and SCN samples. The inventors found that while a proliferation signal is a contributing component, metastatic cases have a significant increase in the expression of the SCN program even when the proliferation component is removed (FIG. 13C). Additionally, in the pancreas, this signature deconvolution separates low grade PNET cases (red box FIG. 13C) from SCN cases, and these cancers are documented to be distinct (Yachida et al., 2012). PNET cases have neuroendocrine features but are indolent and hence have a low “proliferation score”. When the inventors analyzed additional low grade pancreatic PNETs, as well as neuroendocrine tumors of the rectum and small intestine (Alvarez et al., 2018), they both fell in the same pattern of having low proliferation but high neuroendocrine features (FIG. 13D). Taken together, these primary tumor versus metastasis results support that metastatic adenocarcinomas derive elements of their aggressive phenotypes from both a proliferation program and from a program associated with the neuronal programs of SCNCs.

8. Hematopoietic Cancers Share Expression Profiles and Drug Sensitivities with SCN Cancers.

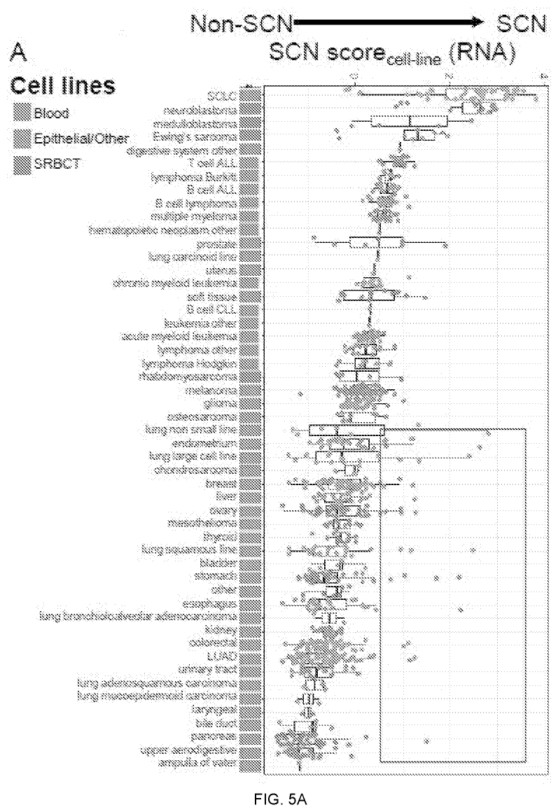

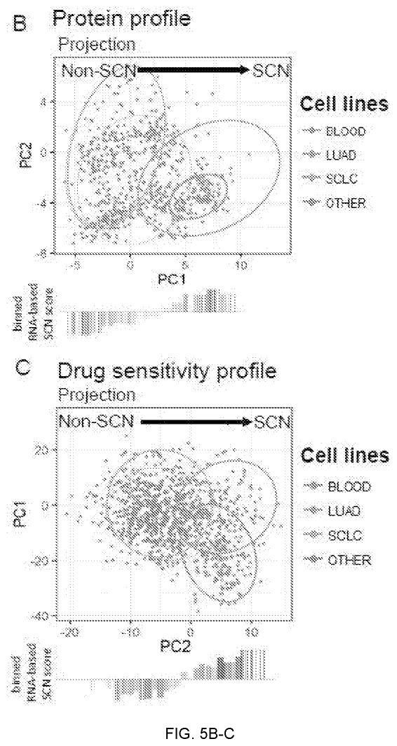

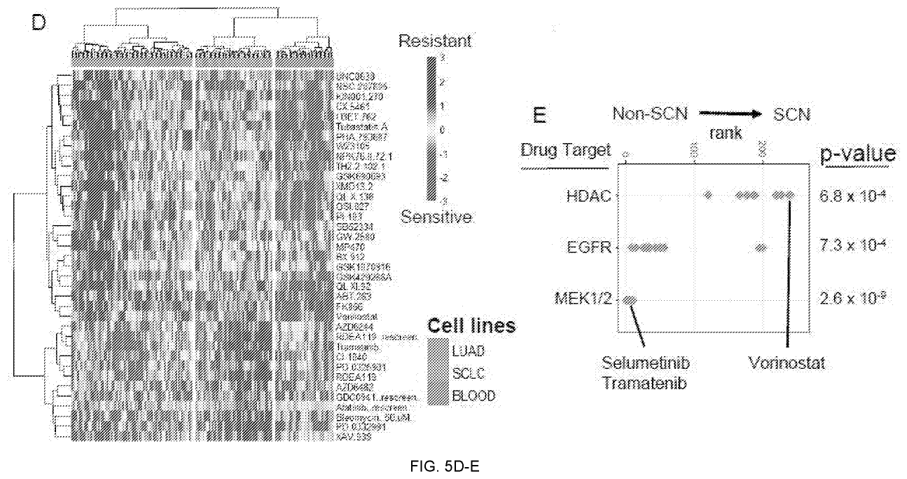

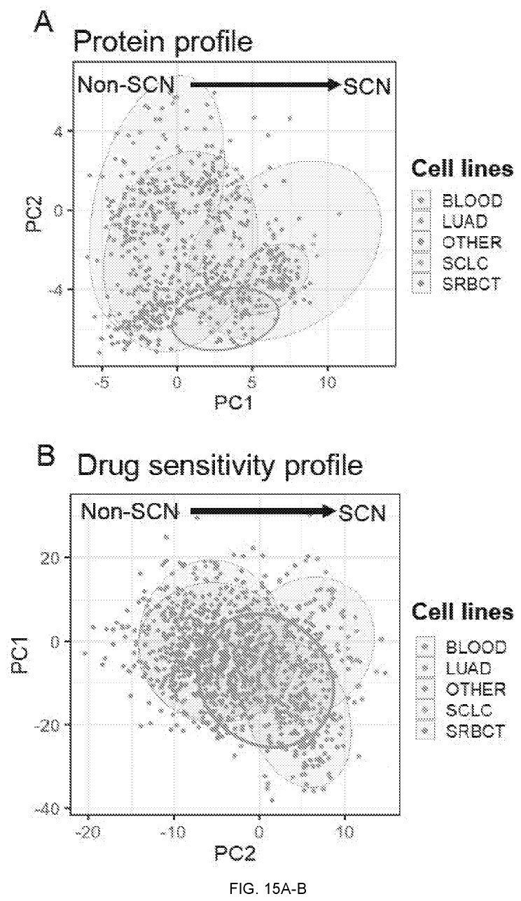

The inventors next leveraged the concordance of SCN tumors and cell lines (FIGS. 8-10) and the availability of drug screen and other data types across cancer cell lines, to gain insight into potential therapeutic vulnerabilities of SCNCs. The inventors scored all CCLE cell lines based on their SCN-gene expression score (based on projection of all lines onto the PLSR space defined by lung adeno and SCN cell lines) (FIG. 5A). Consistent with the tumor findings, i) known SCN cell lines had higher SCN scores than most epithelial lines, and ii) the epithelial cases included a few cell lines that were not annotated as small cell but nonetheless had a strong SCN score that was well into the tail of the distribution of scores for that tumor type (red box in FIG. 5A). Unexpectedly, hematopoietic cancer cell lines had higher SCN expression signatures in comparison to the non-blood epithelial cancers (FIG. 5A), which was likewise observed when the same analysis was performed on tumor data (FIG. 14A).

The inventors next analyzed protein expression signatures using reverse-phase protein array measurements across cell lines from various tissues (Li et al., 2017). The inventors performed PCA on the lung adeno and SCN lines, and found them to be well segregated by this unsupervised protein expression profile-based approach (FIG. 5B). The inventors then projected all other cancer types onto this protein-defined framework. Strikingly, the inventors saw that blood cancers as a group had protein profiles highly similar to lung SCN cancers (FIG. 5B), further supporting the unanticipated similarity in profiles of SCNCs and cancers of the hematopoietic system. The proteins more highly expressed in SCN cancers included the anti-apoptotic factor BCL2, while proteins higher in non-SCN cancers included EGFR, Caspase8, E-Cadherin, and RB (data not shown). To visualize these results in a gene-oriented framework, the inventors used the top differentially expressed proteins between lung adeno and SCN lines to create a clustering-based heatmap of both lung and blood cancer cell lines (FIG. 14B). This framework again found that blood cancer lines clustered with lung SCN lines, with similar increased expression of proteins such as BCL2, and regulators of cell cycle such as ATM, CHK1, and E2F1, which are candidate therapeutic targets in SCLC (Doerr et al., 2017).

Remarkably, the similarity between SCN cancers and blood cancers was seen again when applying PCA analysis to large-scale drug sensitivity data. Here the inventors used a published drug screen of 255 small molecules and common chemotherapeutic agents across a wide panel of cancer cell lines from multiple tissue types (Iorio et al., 2016). Analogous to the protein and gene expression-based analysis, PCA was performed on the matrix of drug sensitivity IC50 values across lung adeno and lung SCN cell lines using all drugs in the screen. This unsupervised approach revealed a unique sensitivity/resistance profile for lung SCN cases compared to lung adenocarcinoma (FIG. 5C). The inventors then projected all other cell lines onto this PCA space. Parallel to the protein-based analysis, blood cancers projected into lung SCN drug sensitivity space, while other epithelial tissue cancer types projected into the lung adeno space (FIG. 5C). Hierarchical clustering based on the drugs with top differential sensitivity between lung adeno and SCN lines also supported the drug sensitivity similarities between lung SCN and blood cancers (FIG. 5D). Compared to lung adeno lines, lung SCN lines were more sensitive to drugs that inhibit histone deacetylation such as Vorinostat (FIG. 5E). Lung SCN lines were more resistant to drugs which target EGFR or components of ERK/MAPK signaling pathways, such as Trametinib and Selumatinib (AZD6244) (FIG. 5E). Taken together, these findings support that cancers of the hematopoietic system have similarities to SCN cancers that range from expression profiles to drug sensitivity-based phenotype profiles.

9. Small-Round-Blue Cell Tumors Share Drug Sensitivities with Lung SCN and Blood Cancers

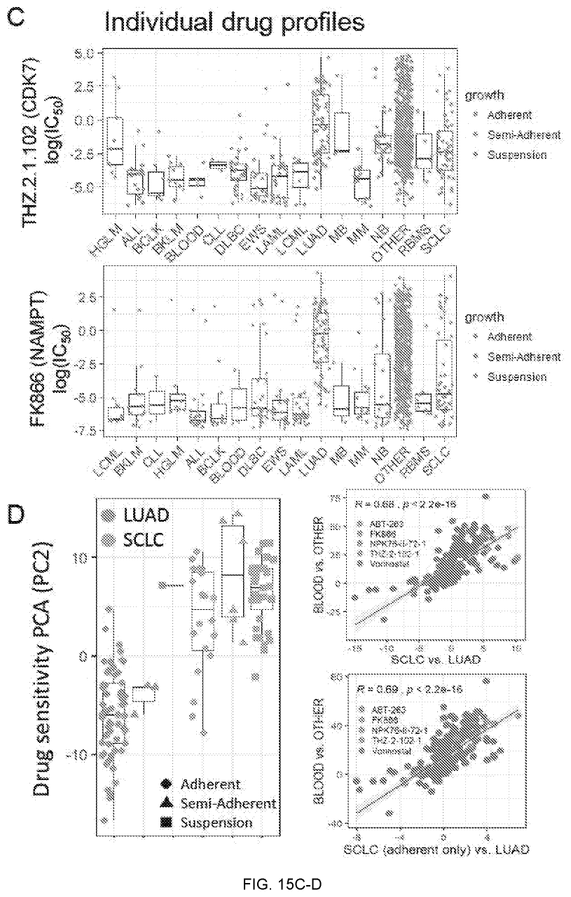

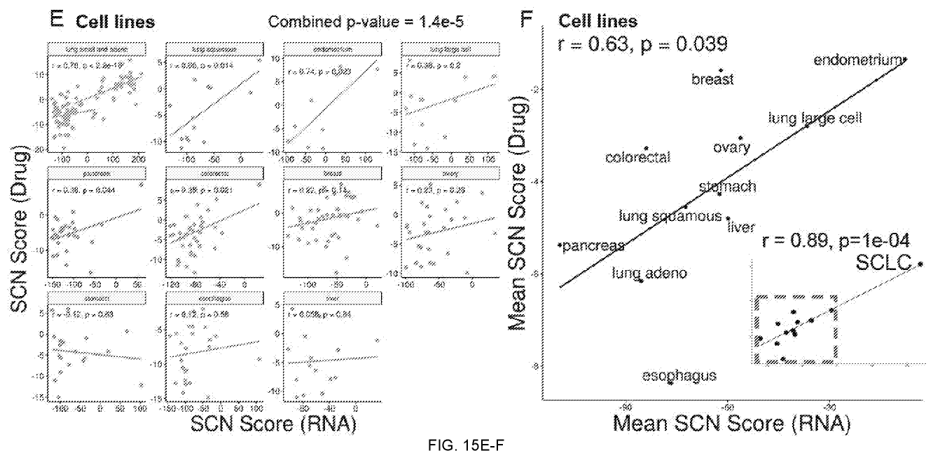

In addition to blood cancers, the inventors' gene signature analysis revealed that small-round-blue cell tumor (SRBCT) cell lines, such as neuroblastoma, medulloblastoma, Ewing's sarcoma, and rhabdomyosarcoma, have high SCN gene expression signature scores (FIG. 5A). These similarities between SRBCTs and SCNCs are also observed in protein profile (FIG. 15A) and drug sensitivity profile analysis (FIG. 15B).

Particularly interesting were drugs with high differential sensitivity in both lung SCN versus adeno, and blood versus non-blood cell line comparisons, supporting that the drug acts through a shared SCN-blood mechanism. The top drugs with such a pan-SCN-blood sensitivity profile included FK866, and THZ-2-102-1 (FIG. 15C). FK866 is one of several NAMPT inhibitors shown to have indications for efficacy in both SCLC and neuronal cancers (Cole et al., 2017; Watson et al., 2009). THZ-2-102-1, a CDK7 inhibitor, targets components of transcriptional regulation and has been shown to be highly effective in MYCN-amplified neuroblastoma (Chipumuro et al., 2014). For many individual drugs, blood cancers of multiple types, SCLCs, and SRBCTs displayed increased sensitivity compared to other epithelial cancer types. Of note, blood cancers and the majority of, but not all, SCN cancers grow in suspension in vitro. However, their common drug sensitivities are not primarily a result of this growth condition. PCA using all drugs in the database indicated that SCLC suspension and SCLC adherent lines are intermingled in their overall drug sensitivity profiles, and are both distinct from LUAD (FIG. 15D). The parallel drug sensitivities of lung SCN and blood cancers (R=0.68) was maintained when the analysis was restricted to the 31% of SCLC lines in the drug sensitivity database that grow adherent in culture (R=0.69; n=20 adherent, n=6 semi-adherent, n=38 suspension) (FIG. 15D). Furthermore, SRBCTs largely grow adherent in culture (n=50 adherent, n=6 semi-adherent, n=6 suspension). Taken together, these results support that the commonalities in drug sensitivities among these three groups are not solely due to suspension culture characteristics (FIG. 15C-D).

10. An Expression-Based SCN Classifier is Predictive of Sensitivity to SCN Targeting Drugs in Non-SCN Epithelial Cancers Across Tissues

In their expression analysis, the inventors found epithelial tumors that had high SCN scores despite not being reported as small cell carcinoma by initial pathology analysis (boxes FIG. 3A). The inventors' cancer cell line analysis revealed analogous cases. More specifically, while epithelial cancer cell lines generally had mean SCN scores lower than blood cancers or SRBCTs, a small subset of epithelial cancer lines had high expression of SCN gene programs well into the positive tail of the distribution (red box in FIG. 5A).

The inventors found that SCN-like cell lines based on expression generally matched SCN-like cell lines based on drug sensitivity profiles (FIG. 15E). The correlation between SCN expression score and sensitivity to SCN-targeting drugs was significant in multiple epithelial tissue types including lung squamous, endometrium, pancreas, and colorectal cancer cell lines. The same trend was seen in 5 of 6 additional epithelial tissue types that did not reach individual significance, and overall the 10 non-lung epithelial tissue types together had a Liptak-Stouffer combined p value of 1.4×10-5. In addition, tissues with a greater mean SCN-expression signature, such as endometrium, breast, and large cell lung cancers, also had drug sensitivity profiles that were closer to that of small cell lung cancer (FIG. 15F).

These drug sensitivity findings support that the predictions based on SCN expression signatures have functional consequences. Thus, not only do cancers of the hematopoietic system and pediatric SRBCTs have similar drug sensitivities to adult SCNCs, but as epithelial cancers from various tissues develop SCN gene expression programs, they become increasingly susceptible to a similar panel of SCN-targeting drugs. These results point to an underappreciated nuance: in addition to distinct cancer types like SRBCTs, blood cancers, or the mixed small cell and non-small cell histology seen in patient biopsies, the cell line data indicates that the small cell phenotype exists along an expression signature-defined spectrum that has influence on the therapeutic vulnerabilities in individual cancers. As such, screening for and targeting the SCN phenotype in individual patient cases could have clinical benefit.

In summary, interrogation and co-analysis of three cell line datasets (gene expression, protein expression profiling, and drug sensitivity data) pointed to three classes of cancer types (blood cancers, SRBCTs, and epithelial SCN-like cancers) with SCNC-like molecular profiles. These concordant molecular profiles had functional consequences and revealed specific shared drug vulnerabilities.

11. Validation of Shared SCN and Blood Susceptibilities Based on Gene Dependencies

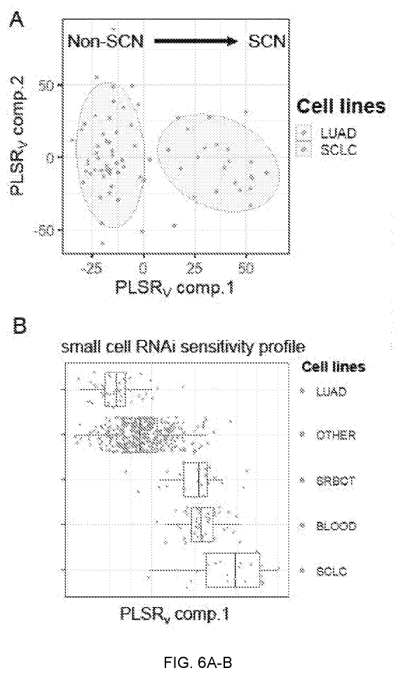

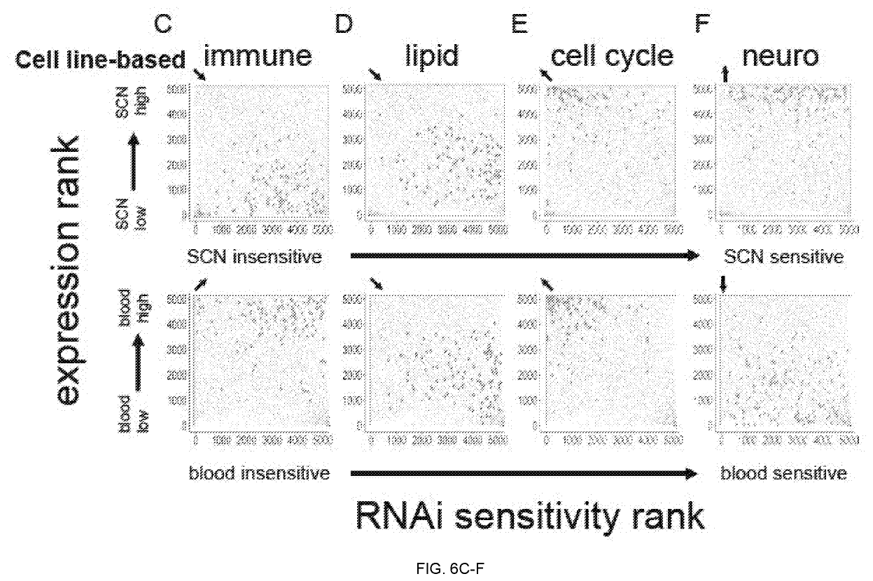

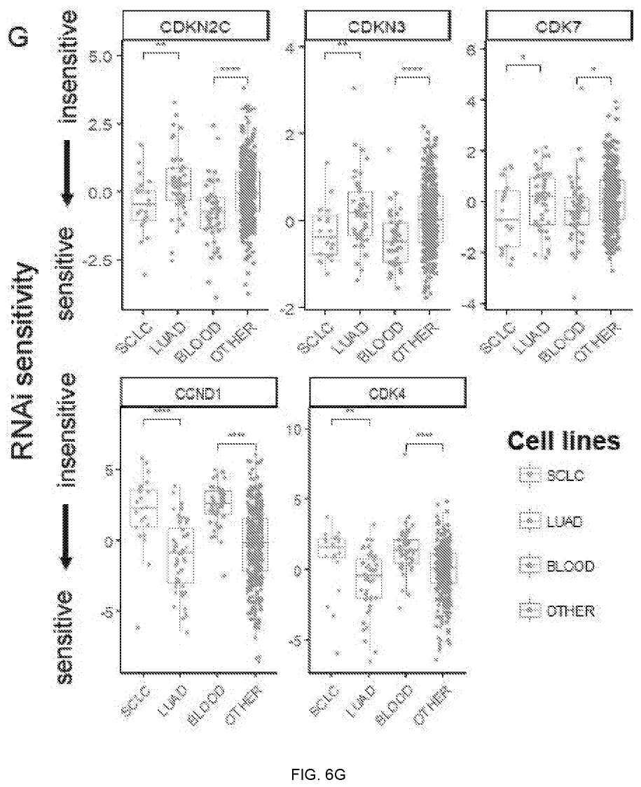

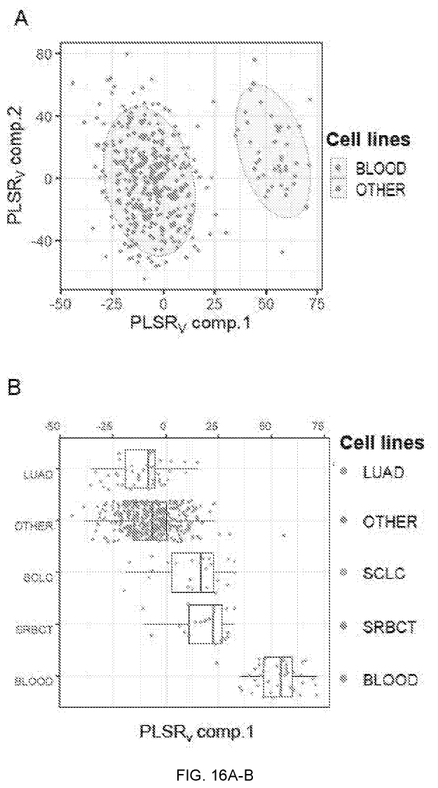

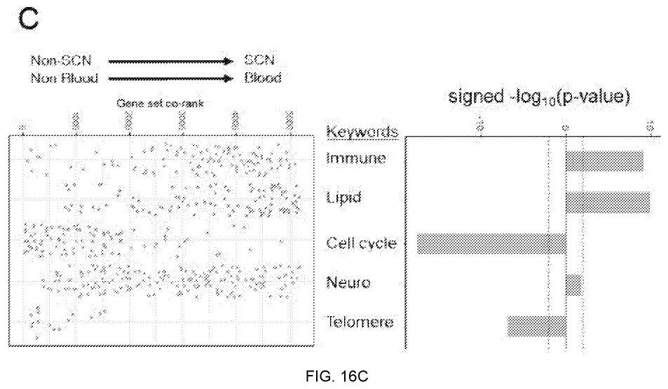

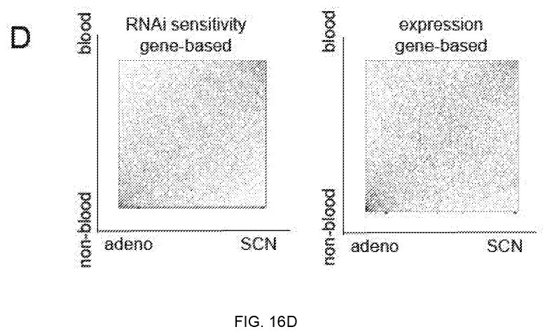

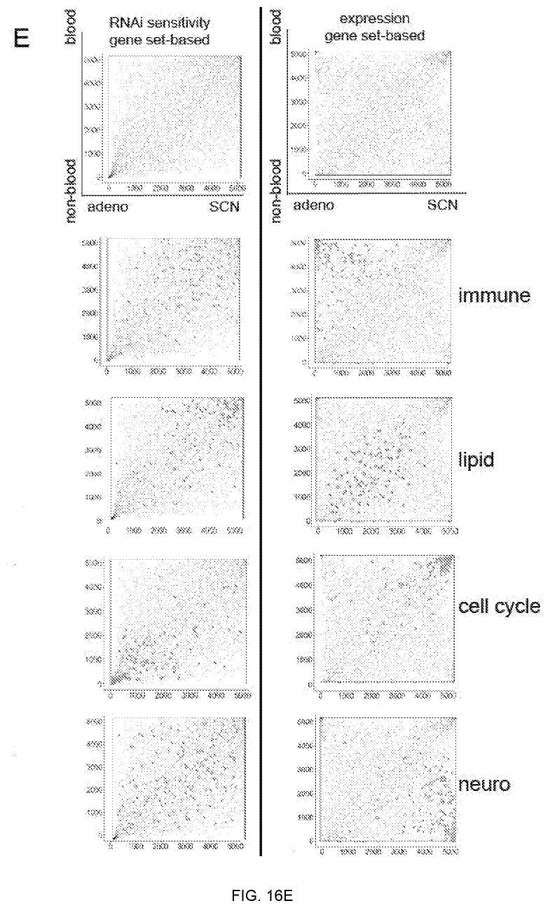

In that drug susceptibility profiles implicated shared phenotypes between SCN and blood cancers, the inventors next sought to validate these findings using an independent empirical dataset. Towards this end, the inventors analyzed a genome-scale RNA-interference (RNAi; shRNA-based) functional screen across a large panel of cell lines from various tissues (Tsherniak et al., 2017). The inventors first trained a prediction model on the gene dependency data between lung adeno and SCN cell lines (FIG. 6A). A PLSR-based susceptibility prediction approach had 92% cross-validation accuracy for predicting the identity of lung cell lines that have concordant annotation and gene expression signatures (data not shown). Applying the prediction model to the remaining cell types showed that blood and SRBCT lines share gene depletion-defined susceptibilities with the lung SCN lines (FIG. 6B). In reverse, using a blood versus non-blood RNAi sensitivity framework as the predictor confirmed that SCNCs and SRBCT's have more blood-like sensitivities (FIG. 16A-B). Thus, the RNAi dependency framework validates the inventors' drug-based findings that SCNCs, hematopoietic cancers, and SRBCTs have shared susceptibilities.

Investigating the biological pathways that had shared knockdown susceptibility in SCN and blood cancer cell lines, revealed that both cancer types were enriched in sensitivity to disruption of immune pathways and lipid and sterol metabolism (FIG. 6C-D, FIG. 16C). Notably, this susceptibility enrichment was not exclusively tied to elevated (nor depressed) gene expression. In fact, SCN cell lines have lower expression of immune pathway genes but remain sensitive to their disruption. In contrast, SCN and blood cancer cell lines share increased expression of cell cycle pathway genes, but both had decreased sensitivity to the knockdown of these genes (FIG. 6E). Although SCNCs had high expression of neural genes (FIG. 1), and a few distinct neural gene sets did show genetic sensitivity to knockdown (FIG. 6F upper panel), there was no substantial enrichment of susceptibility to knockdown of neural genes—supporting that many components of the upregulated neuronal gene programs promote phenotypes distinct from the regulation of cell survival (FIG. 6F). These expression versus sensitivity results highlight that cells are not particularly dependent on genes with elevated expression. For example, blood and SCN cancers shared an expression-based proliferation profile, but gene dependency data did not support proliferation as the most direct determinant of shared blood and SCN vulnerabilities (FIG. 6E). Furthermore, even the knockdown of genes with reduced expression can have functional consequences.

While the inventors' transcriptome, proteome, drug sensitivity, and gene susceptibility analyses above uncovered many similarities between SCNCs and blood cancers (FIG. 16C-D), naturally there are also many differences, especially at the gene expression level with blood cancers expressing more immune-annotated genes, and SCNCs expressing more neuro-annotated genes (FIG. 16E).

The inventors further investigated specific knockdown sensitivities of genes encoding protein targets currently of interest in clinical trials for various cancer types. The inventors focused on CDKs and CDK antagonists, which had strong differential sensitivities in the lung adeno versus small cell, and blood versus non-blood comparisons. CCND1 and CDK4 knockdown were more effective in lung adeno compared to either SCLC or blood cancers, while knockdown of CDKN2C, a CDK4 inhibitor, was more effective in SCLC and blood cancers (FIG. 6G). The CCND1-CDK4 complex has been shown inhibit phospho-RB to promote cell cycle progression, and loss of RB function is a hallmark in the development of the SCN phenotype (FIG. 10G & FIGS. 12A-B). These results predict that CDK4i-treated epithelial cancers could use de-differentiation to an SCN phenotype as a resistance mechanism. This finding is consistent with RB loss as a resistance mechanism to CDK4 antagonism observed in preclinical models of liver cancer and glioblastoma, and in patients with metastatic breast cancer (Bollard et al., 2017; Condorelli et al., 2018). Notably, CDK7 knockdown was more effective in SCLC and blood cancers, paralleling the drug sensitivity finding for THZ-2-102-1, a CDK7 inhibitor (FIG. 6G, FIG. 15C). Thus, CDK7 sensitivity provides a specific example of a previously documented sensitivity in both SCNCs and blood cancers (Cayrol et al., 2017; Christensen et al., 2014), that is a part of the wider panel of shared sensitivities revealed by the inventors' pan-cancer and functional screen analysis.

12. Concordant Gene Expression-Based Drug Sensitivity Profiles in SCN and Blood Tumors

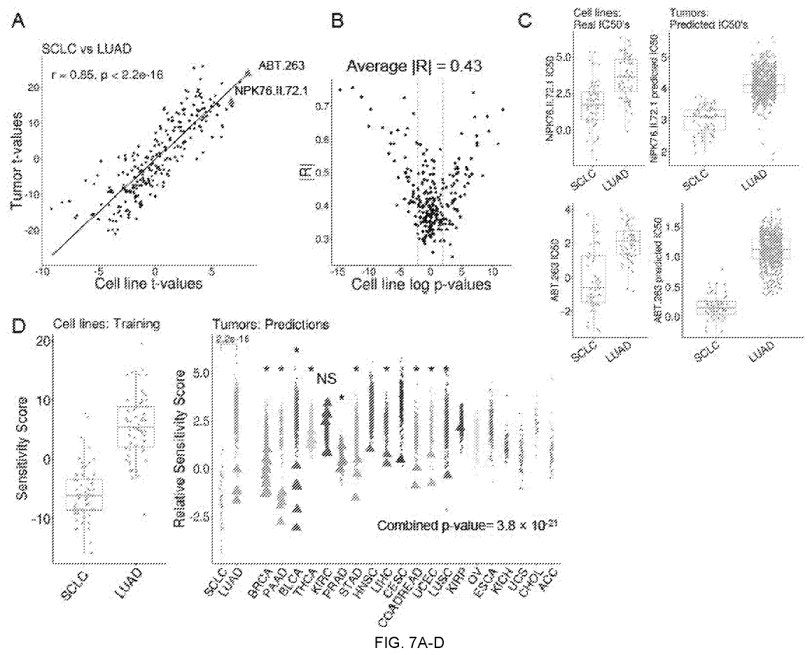

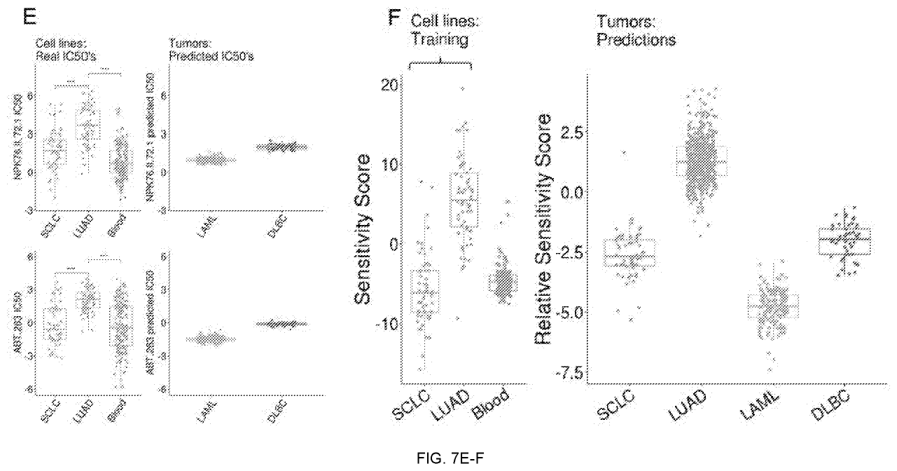

The inventors next investigated if the gene expression profiles associated with drug susceptibilities are present in primary tumors. Using an elastic net regression framework, the inventors built a predictor of drug sensitivities based on the lung adeno (LUAD) and lung SCN (SCLC) cell line gene expression profiles. The inventors applied this cell line-trained predictor to the expression profiles of tumors. In general, drugs that were differentially potent in adeno versus SCN lung cancer cell lines, were likewise predicted to be differentially potent in adeno versus SCN lung tumors (average |R|=0.43) (FIG. 7A-B). This result included a drug that targets BCL2 (ABT.263), a gene which is often overexpressed in SCLC as a resistance mechanism to etoposide treatment (van Meerbeeck et al., 2011); and the PLK inhibitor NPK76.II.72.1 (FIG. 7C, red dots FIG. 7A). To determine the global sensitivity status of individual tumors, elastic net-predicted tumor sensitivities were projected onto the PCA drug sensitivities of lung adeno and SCN cancer cell lines (as shown in FIG. 5C). The inventors found that epithelial tumors with (previously unannotated) SCN features (based on gene expression profiles, FIG. 3A), typically had drug sensitivity profiles more similar to annotated SCLC tumors than to LUAD tumors (FIG. 7D). 10 of 11 tumor types had a significant association of expression-based predicted SCN status to predicted SCN-like sensitivity (KS test-based; FIG. 7D). These results support that the expression profiles associated with drug sensitivities in vitro are reflective of the in vivo tumor setting.

The inventors then expanded this analysis to include hematopoietic tumors. As in the cell line empirical-sensitivity data case, blood tumors shared predicted-drug sensitivity profiles with SCN tumors. These shared profiles included drugs that target BCL2 (e.g., ABT.263) which are already approved in chronic lymphoblastic leukemia (CLL), carry breakthrough designation for acute myelogenous leukemia (AML), and are in various stages of therapy development for other hematological malignancies (Seymour et al., 2018) (FIG. 7E-F). Thus, the inventors observe a previously unappreciated similarity between SCNCs and blood cancers that spans gene and protein expression, and drug sensitivity and gene vulnerability profiles. Importantly, the sensitivity profiles demonstrate predicted concordance in tumor data, raising the potential for developing SCN-targeted therapeutics from the established knowledge base for clinical treatment of hematopoietic malignancies.

C. Discussion

In this work, by defining the molecular signatures shared between SCN cancers from different tissue types, the inventors identified molecular networks, key transcription factors, and gene mutations that drive a convergent SCN phenotype. The strength of these shared profiles allowed the inventors to define a molecular classifier that scores a spectrum of intermediate and outlying SCN-like cancers in patient biopsies not currently identified by histology or limited biomarker sets alone. Survival analysis supports the aggressiveness of these SCN-like epithelial cancers (FIG. 3B) (Chen et al., 2018). The implications of identifying this molecular spectrum of SCN cancers is apparent in functional data on drug response and gene dependency, which further supports the value of identifying SCN-like cases in clinical assessment. The inventors found three categories of tumors to generally share expression and sensitivity features with pathology-defined SCN cases: (i) SCN-like epithelial cancers, (ii) pediatric small round blue cell tumors (SRBCTs), and (iii) hematopoietic malignancies.

The inventors' molecular profiling-based SCN scoring identified cases with SCN or neuroendocrine features across numerous purportedly non-SCN primary tumors. These cases may not have been reported in the original pathology reports due to the fact that their SCN component is typically focal, and the conventional thinking that SCN is rare in breast and other epithelial tumors. Epithelial tumor types, such as breast cancer, in which SCN tumors rarely occur but have poor prognosis (Inno et al., 2016), present statistical challenges in advancing care through clinical trials, and thus cross-tissue learning supported by shared molecular profiles will likely be required. In sum, the inventors' findings of shared molecular profiles, and shared drug and genetic sensitivities support and guide efforts to predict rare SCNC cases and develop therapies that will target SCNCs from multiple tissue sites of origin.

The cell of origin for SCN cancers is not universally defined. SCN cancers have been alternatively reported to arise from primary normal neuroendocrine cell precursors in some cases, and transdifferentiation from adenocarcinoma in others (Watson et al., 2015). The cell of origin may vary depending on factors such as whether the SCN cancers arise de novo or as a resistance mechanism to therapeutics (Feng et al., 2017; Rickman et al., 2017). In either case, the transdifferentiation-linked path to an SCN cancer converges, and thus leads to molecular profiles and phenotypes increasingly independent of the tissue of origin. This model of convergence is supported by the inventors' demonstration that normal prostate and lung cells can be reprogrammed into prostate and lung SCNCs, respectively, using a common set of small cell cancer-associated genetic perturbations (Park et al., 2018). Despite using distinct cell types as the starting material, the resulting tumor models had highly similar molecular profiles, furthermore matching the overlapping profiles of patient prostate and lung SCNCs. Combination therapies that both treat non-SCN cancers and prevent escape towards SCN cancers may help curtail the incidence of the disease. The pan-tissue scope of shared SCN susceptibilities supports the potential for pan-cancer therapy development to impact a large number of otherwise aggressive, metastatic, and treatment-resistant cancers.

D. Methods

1. Data Acquisition and Processing

Gene expression data was obtained from TCGA (available online at //xenabrowser.net/hub/), (George et al., 2015)(SCLC data), (Beltran et al., 2016) (CRPC/NEPC data). (Robinson et al., 2017) (MET500 data), and all lung lines in the Cancer Cell Line Encyclopedia (CCLE) (Barretina et al., 2012). To minimize batch effects, the inventors uniformly processed the raw FASTQs through a single analysis pipeline (TOIL) (Vivian et al., 2017). CCLE lung cell lines were processed through TOIL when comparing to TCGA samples which were also processed through TOIL. CCLE data on all cell lines was obtained in a format pre-processed through Salmon (Patro et al., 2017) (https://ocg.cancer.gov/ctd2-data-project/translational-genomics-research-institute-quantified-cancer-cell-line-encyclopedia), and all analyses done on CCLE cell lines only were performed using this data from this processing pipeline (FIG. 5A). The CLCGP lung cancer microarray gene expression dataset used to build the ARACNe network was downloaded from www.uni-koeln.de/med-fak/cicgp. Upper quartile normalized expression values were transformed to [(log)]_2 (x+1).

Methylation data was obtained from TCGA, (Iorio et al., 2016) (GSE68379), Beltran et al. (dbGaP phs000909.v1.p1), and (Mohammad et al., 2015) (GSE66298). The 450K array data (from TCGA, Iorio et al., and Mohammed et al.) was obtained in processed form. Beltran et al. reduced representation bisulfite sequencing (RRBS) data was obtained in FASTQ format, and aligned to hg38 using bwameth (Pedersen et al., 2014). Methylation metrics were called using MethylDackel, which groups cytosines into one of three sequence contexts: CpG, CHG, or CHH. Only cytosines in CpG context were used for downstream analysis.

Cell line annotation was performed by harmonizing the annotation of the CCLE, GDSC, Demeter, and Ceres datasets. Cell line annotations were cross checked with ATCC (https://www.atcc.org/), Cellosaurus (https://web.expasy.org/cellosaurus/), and DSMZ (https://www.dsmz.de/) when possible. When discrepancies arose between these 3 online sources and the primary annotation, the inventors defaulted to the online sources, which have stringent analysis pipelines. Lines with problematic annotation as defined by Cellosaurus were left out of the analyses. Cell line culture growth characteristics (e.g. adherent vs suspension) were obtained from the Dependency Map database (available online at //depmap.org/portal/) and (Iorio et al., 2016).

2. Quantification and Statistical Analysis

a. PCA/PLSR

Log 2 transformed upper quartile normalized expression of coding genes was used to perform unsupervised principal component analysis (PCA). This method uncovers latent components which are a linear combinations of the features that most strongly vary across the datasets. PCA was performed centered and unscaled using the prcomp function in R. Varimax rotation of the PCA or PLSR loadings was performed on 2 components, without Kaiser normalization, using the R varimax package. Projections onto varimax-rotated PCA/PLSR frameworks were done by multiplication of the original projected sample scores by the varimax rotation matrix. When applicable (e.g. FIG. 1A) TCGA cancers (LUAD, PRAD, and BLCA) were randomly down sampled to more closely match the number of samples in data sets from George et al. and Beltran et al.

b. Small Cell Neuroendocrine (SCN) and Proliferation Scores

i. Tumors

For RNAseq data using patient tumors, “SCN score” is the PC1 score after projection onto the FIG. 1A varimax PCA framework. Because of the nature of PCA, this score is determined as a linear combination of weights that includes every coding gene, and hence is not strictly dependent on solely one subset of genes. After projection onto this framework, the score was either z-scored in each individual tumor type as in FIG. 3A (to highlight outlier samples), or left un-zscored to place cancers on a common scale as in FIGS. 4A, 4C, and S7A. Samples greater than 3 standard deviations from the mean in the z-scored analysis were deemed “SCN-like” (e.g. FIG. 3A, FIG. 3B).

As SCN cancers are highly proliferative and display neuroendocrine features the inventors sought to de-convolve these two influences on SCN score. To that end the inventors created a “SCN minus proliferation score”. A list of proliferation genes was generated from the union of three lists of proliferation genes published by Benporath et al, Cyclebase (https://cyclebase.org/), and KEGG cell cycle genes. PCA was performed using only these proliferation genes, and the absolute value of each gene's Pearson correlation to PC1 was calculated. A ROC curve was created using two classes with the pROC package to choose a threshold cutoff (Youden's J statistic) for genes highly correlated with proliferation. Correlated genes above threshold and all annotated proliferation genes from the original list were removed. PCA and GSEA-squared analysis (see below) was then redone. “SCN minus proliferation score” is thus the PC1 score after sample projection onto the varimax PCA of the samples used in FIG. 1A with this new gene list. A “Proliferation Only Score” was also created using the union of genes in the three lists and those removed using the ROC curve method. These proliferation-related genes were used to create another varimax PCA of the samples in FIG. 1A. Proliferation Score is the varimax PC1 score after projection onto this framework. The proliferation-removed score was used in the analysis of the breast cancer slides to highlight samples that had a high probability of having neuroendocrine features. This method additionally has utility in distinguishing between SCN samples which have both neuroendocrine and proliferative features, from the indolent primitive neuroectodermal tumors which have neuroendocrine features but lack a proliferative signature. The proliferation-removed score was used in FIG. 3I, 11I, S6C-D. For FIGS. 3I and 11J, the proliferation-removed score was z-scored, since only BRCA tumors were involved in these panels. For FIG. 13D, both the x and y-axes were left un-z-scored to place all samples on a common scale.

ii. Cell Lines:

For cell lines, RNA and RNAi sensitivity based SCN scores were calculated by projection onto the varimax PLSR framework of the SCLC/LUAD dichotomy. For both these data types, SCN Score is the “component 1” score after projection onto this framework. Samples with annotation not concordant with their expression-based predictions, from a linear-discriminant Leave-one-out Cross Validation analysis using the first three components of an expression PCA, were removed in the RNAi susceptibility-based PLSR. For protein data, the “SCN score” is the PC1 score of a sample upon projection onto the protein data-based framework of the SCLC/LUAD dichotomy (e.g. FIG. 5B). For drug sensitivity, as the distinction between non-SCN (LUAD) and SCN cases (SCLC) is on PC2, the drug sensitivity “SCN score” is the PC2 score of a sample after projection onto that framework (e.g. FIG. 5C).

iii. Transcription Factor Analysis

ARACNe (Lachmann et al., 2016b) network connections were created using all genes, and then the network nodes were restricted to 1675 transcription factors (TFs) by combining all TF gene sets in the GO gene ontology. One network was built for each of lung (CLCGP et al., 2013), prostate (Beltran et al., 2016), and bladder (TCGA), using a balanced set of samples from the SCN and adenocarcinoma groups when possible, with default settings. VIPER analysis (Alvarez et al., 2016) was performed using the msviper function from R package viper, with a minimum network size of 10. The combined p-value across the three tissues was calculated using Stouffer's method by converting two-way p-values from msviper into one-way p-values using the two2one and sumz functions from the metap package in R.

iv. GSEA-Squared

Gene Set Enrichment Analysis (Subramanian et al., 2005) was done on pre-ranked lists of genes using the MSigDB C5 gene sets and Kolmogorov-Smirnov (KS)-based statistics. In order to identify categories of genesets that could be enriched or de-enriched, all individual words in the genesets were collected and their frequencies were tabulated. Words with frequencies<5 or >500 were excluded. All genesets were then ranked by their NES value. Using genesets containing each particular word, all the individual words were then ranked by their KS test p-value using ks.test.2 at (https://github.com/franapoli/signed-ks-test). Top words with small p-values were considered categories of interest, such as ‘immune’ or ‘neuron’. A manual curation of other top words related to the categories, and inspection of gene sets containing these words, was done to group related gene sets. The keywords used for each category are listed below:

| Category | Keywords |
|---|---|
| Neuro | NEURO, SYNAP, VOLTAGE, AXON, CEREBRAL, |
| | CORTEX |
| Cell cycle | CELL_CYCLE, MITOTIC, DNA_REPLICATION, |
| | CHROMOSOME_SEGREGATION, SPINDLE, |
| | CELL_DIVISION |
| Splicing | SPLICING, SPLICESOME, SPLICESOMAL |
| Immune | INFLAM, IMMUNE, IMMUNITY, INTERLUEKIN, |
| | LEUKOCYTE |
| Adhesion | ADHESION, ADHERENS |

All gene sets were then ranked by their Normalized Enrichment Score (NES), and KS tests were performed to assess the distribution of gene set categories using ks.test.2. This second application of KS test-based enrichment analysis led to the coining of ‘GSEA-squared,’ enrichment analysis first on genes, and then on geneset categories. For multiple-hypothesis testing of KS tests of enrichment in FIGS. 6C-F, each category's one-way p-values were corrected using the Benjamani-Hochberg method placing them within the full list of keywords.

vi. REST Analysis

REST/NRSF is transcriptional repressor and restricts neural gene expression. The inventors used an aggregate measure of REST activity by calculating a “REST Score” for each sample from RNAseq data. “REST Score” for individual samples was determined using the gene set NRSF_01 from MSIGDB (http://software.broadinstitute.org/gsea/msigdb/geneset_page.jsp?geneSetName=NRSF_01) which contains genes that have a 3′ UTR motif that matches annotation for the REST transcription factor. In this model a higher “REST Score” corresponds to de-repression of REST target genes, and hence conditions that support more neural gene expression, For each sample, a “single sample gene set enrichment score” (ssGSEA) was determined using the V$NRSF_01 gene set. REST gene expression and “Rest Score” were correlated to “SCN Score” in individual cancer types using the Pearson's correlation (Barbie et al., 2009).

vii. Rank Rank Hypergeometric Overlap (RRHO)

Rank Rank Hypergeometric Overlap was performed using the online tool and the R package RRHO, with step size 100 for expression data and methylation gene-based data, and 2000 for methylation probe-based data (Plaisier et al., 2010).

viii. Methylation Analysis

Methylation levels were expressed as β-values, indicating the overall proportion of methylation at each particular site [(methylated/(methylated+umethylated)]. PCAs were performed centered and unscaled on the entire data matrix. The IlluminaHumanMethylation450k.db package was used to provide annotation information on the location of the probe in relation to regulatory elements. Tissue-agnostic enhancer locations were provided by the IlluminaHumanMethylation450k.db package (Tim Triche, Jr., 2017), which informatically determines enhancer probes using ENCODE data. For the site-based analysis, PLSR was run individually on lung and bladder tissues, regressing against the phenotype of SCN or non-SCN. Non-SCN samples for lung and bladder were down-sampled to more closely match the number of SCN samples. To test for the importance of OpenSea sites in contributing to the SCN versus non-SCN distinction, the absolute value of the loadings was ranked, and 1-sided KS test was performed against a background of all sites. To incorporate prostate methylation reduced-representation bisulfate sequencing (RRBS) data with the lung and bladder 450K array data, the number of sites was reduced to sites represented across both platforms. Sites were then further collapsed to genes by matching probes to genes using IlluminaHumanMethylation450k annotation. Probes that matched to multiple genes based on the Illumina annotation were removed. Averaged methylation values for each gene were then ranked by PLSR loadings on each tissue type. These loadings from the three tissue types were averaged and GSEA was performed on this ranked list. The gene-based summarization of methylation data was also performed on the lung cell lines and PLSR was performed. The equivalent methylation data format from tumors was projected to the cell line framework.

ix. CNA Analysis

TCGA SNP6.0 Affymetrix derived seg files were downloaded from the GDC repository. Cell line seg files were created using RAWCOPY from the .cel files with default settings (Mayrhofer et al., 2016). Seg files were inputted into GISTIC2.0 to obtain both thresholded calls and continuous log 2 CNA values mapped to genes. PCA was performed uncentered and unscaled on the continuous log 2 CNA data. IGV was used for visualization. To obtain concordant CNA regions across lung and prostate small cell samples, PLSR was performed on lung and prostate tissues separately, with 1 and 0 representing SCN and non-SCN samples, respectively. For lung, a random sample of LUAD samples and all SCLC samples were used. For prostate, the samples were subset to include only one sample from each patient. One region containing highly focal CNAs on Chromosome 1 were removed by inspection of prostate PCA loadings, because they vastly dominated the top components' loadings of the PCA analysis and were likely technical artifacts. Relative amplification or deletion regions that were consistent across lung and prostate tissues were retained (defined strictly by commonly positive or commonly negative CNA PLSR loadings values). The loadings for consistent regions were averaged; non-consistent regions were set to 0.

Integrated CNA (iCNA) score (Graham et al., 2017) for each sample was defined as:

$$i C N A = \frac{{\sum }_{segments} \vert segment end - segment start \vert \times \vert segment mean \vert}{♯ base pairs in sample}$$

c. Pathology Analysis

To evaluate the molecular profile (gene expression)-based SCN predictions, a pathologist evaluated the available TCGA slide images located at http://cancer.digitalslidearchive.net/. Typically, each TCGA case has sections from formalin-fixed paraffin-embedded (FFPE) tissue and from frozen tissue. To avoid artifacts associated with frozen sections, only FFPE sections were used for histology classification. For the pan-cancer histology analysis, 16 samples with high SCN signature score from multiple tissue types were analyzed by a pathologist. Representative images of neuroendocrine and non-neuroendocrine regions were obtained by taking screen shots. From the breast cancer (BRCA) cohort, 38 samples were selected to include 1) the majority of SCN high cases, 2) a random sampling of tumors with middling SCN scores, 3) tumors with low SCN score, and 4) samples with high expression levels of at least one of four traditional SCN markers, CHGA, SYP, NCAM1, and TTF1. Each digital slide was divided into 4 roughly equal quadrants to ensure that the slide was examined evenly across different regions. In each quadrant, 20 non-contiguous areas were examined at low, medium and high powers to determine the histologic features of the tumor. Representative images were obtained at the highest magnification (400×) by taking screen shots. Cases were classified as mixed tumors when two different histologic types (most commonly, invasive ductal carcinoma and small cell neuroendocrine carcinoma) co-existed. For a case to be classified as a mixed tumor, the minor component (usually, small cell neuroendocrine carcinoma) needed to occupy a substantial area of the tumor. Specifically, the inventors required that the tumor cells of the minor histology should coalesce in a region that is equal to or larger than 2 high power fields. To determine if pathology based-SCN positive cases were enriched in samples with high SCN score, samples were rank ordered by SCN score and a Kolmogorov-Smirnov enrichment test was performed on pathology-based SCN status (Table S5). Pathology website with all detailed images is located online at //systems.crump.ucla.edu/scn/.

d. Survival Analysis

For the pan-cancer analysis, Cox regressions were performed based on SCN-like/non-SCN status, controlling for cancer type using the coxph function in R. Survival on a continuous scale was performed based on the SCN score (PC1 score) determined by projection onto the FIG. 1A plot, controlling for cancer type using the coxph function in R. Wald-test p-values were reported.

e. Mutation Analysis

Mutations were assembled from the MutsigCV2 calls for each tumor type from firebrowse.org. The gene list used was the union of recurrent mutations (mutsigcv2 q-value<0.1) from all cancers in the TCGA. To determine the association of mutations with the neuroendocrine phenotype a generalized linear model (GLM) was used. In the analysis of cancers with known SCN cases (FIG. 12A) a logistic regression was conditioned on the SCN score and the tissue type on the combined data from “prostate” (consisting of NEPC, CRPC, PRAD) and “lung” (SCLC, LUAD). For the pan-epithelial cancer analysis (FIG. 12B) the logistic regression was conditioned on SCN score and cancer type. In the individual cancer cases (FIG. 12C) a Wilcoxon rank sum test on neuroendocrine score of mutant vs non-mutant was used. Multiple hypothesis correction was performed with the Benjamani-Hochberg method.

f. RPPA Analysis

RPPA data was obtained from (Li et al., 2017). As this data has missing values, imputation was performed using probabilistic PCA, using the ppca and completeObs functions in the pcaMethods package (Stacklies et al., 2007). LUAD and SCLC cell lines were processed together in one batch without other cell lines so as not to intermix test and training sets. Missing value imputation for all other cell lines were performed together in one batch. Prior to each imputation the inventors removed all proteins with greater than 25% missing values in that batch. Samples were projected onto the PCA of the imputed values SCLC and LUAD (FIG. 5B).

g. Drug Sensitivity Analysis

Drug sensitivity data (log IC50 values) was obtained from GDSC (Iorio et al., 2016). For LUAD and SCLC cell lines, differential sensitivity to each drug was calculated using the Student's t-test. These drugs were then ranked by the t-test statistic. Annotation on drug target and target pathway was obtained from the pharmacogenomics screen published by Iorio et al. From the annotation on drug targets, a list of genes or biological targets was obtained. For each target, a KS test was performed (bootstrap n=1000) on the t-test ranked list of drugs that contained that target in its annotation, resulting in a list of targets significantly enriched or de-enriched for small cell cancer sensitivity. Missing values in the drug sensitivity data were imputed using the weighted average of k-nearest neighbors (n=3). For SCLC and LUAD samples that were used as training data, k-Nearest Neighbors (kNN) imputation was performed on the lung samples alone. kNN imputation was then then performed on all other cell lines together, excluding the SCLC and LUAD samples. PCA was performed on the imputed data. The inventors then projected the drug data for all lines onto the PCA defined on lung SCLC and LUAD, including cancer of hematological and neuroectodermal origin. Projected points (all lines excluding lung SCLC and LUAD) in the drug sensitivity plot were annotated by their expression projection values. These values were binned along the x-axis and mean expression projection values were summarized in the corresponding waterfall plot (eg. FIG. 5B, FIG. 5C). Combined p-values were obtained by the weighted-Stouffer test with the one way null hypothesis that the Pearson correlation is less than zero; using the lower tail of the t-distribution for values outputted by cor.test in R (lung small cell not included). The square root of individual group sample sizes were used as weights to stabilize the variance of the mean in this calculation (Zaykin, 2011).

h. Gene Dependency

shRNA data was taken from the Achilles Project (Tshemiak et al., 2017). Demeter gene dependency scores were used. A lower Demeter score indicates sensitivity to downregulation of that gene. PLSR was performed on 1) LUAD and SCLC lines, and 2) blood and non-blood lines (all cell lines except SRBCT, LUAD, or SCLC lines). Varimax rotation was performed on 2 components. Other cell lines were projected onto the lung or blood PLSR frameworks using the varimax-rotated loadings. Student's t-test was performed on lung adeno versus lung SCN lines, and blood versus non-blood lines, producing two gene lists, each ranked by p-value representing differential RNAi sensitivity in the two above comparisons. RRHO was performed on these two ranked lists of genes, and on their corresponding GSEA-analyzed genesets ranked by NES score. For each gene, the sum of the lung adeno versus SCN rank and blood versus non-blood rank for each gene was calculated, and the list was co-ranked using this sum. Enrichment analysis (GSEA) was performed on the co-ranked list of genes using the MSigDB C2: KEGG, C2: Reactome, and C5: all GO gene sets. GSEA-squared (see above) was performed by ranking individual words by their signed KS test p-value using ks.test.2. The keywords used for each category are listed below:

| Category | Keywords |
|---|---|
| Telomere | TELOMERE |
| Immune | INFLAM, IMMUNE, IMMUNITY, INTERLUEKIN, |
| | LEUKOCYTE |
| Cell cycle | CELL_CYCLE, MITOTIC, DNA_REPLICATION, |
| | CHROMOSOME_SEGREGATION, SPINDLE, |
| | CELL_DIVISION |
| Lipids | COA, LIPID, STEROL |
| Neuro | NEURO, SYNAP, VOLTAGE, AXON, CEREBRAL, |
| | CORTEX |

i. Tumor Drug Sensitivity Prediction

Tumor drug sensitivity prediction were performed using elastic nets with the caret package in R, using cell line RNAseq and IC50 drug sensitivity data. For each drug two distinct models were created. 1) Using the 1000 most variable genes at the RNA level to predict drug sensitivity in the SCLC and LUAD cell lines (used in FIGS. 7A-D), and 2) using 1000 of the most variable genes across all cell lines (used in FIG. 7E-F). This resulted in a total of 510 models (255 drugs each). Using the predicted values, tumors were projected onto the PCA of real drug sensitivity values shown in FIG. 6C. The relative sensitivity scores are the PC2 values from this projection.

E. Tables

TABLE S5-A
Pan-Tissue Cancers Pathology Calls

| | “SCN | | |
|---|---|---|---|
| | minus | | |
| | proliferation” | | |
| | score | | |
| | (PC1; | Cancer | |
| Sample | x-axis) | Type | Pathology Call |
| TCGA-A6- | 6.852 | COADREAD | Moderately differentiated adenocarcinoma |
| 5656 | | | with extensive neuroendocrine differentiation |
| TCGA-XQ- | 4.875 | PRAD | Mixed prostate adenocarcinoma and small |
| A8TA | | | cell neuroendocrine carcinoma |
| TCGA-86- | 4.809 | LUAD | Mixed poorly differentiated lung adenocarcinoma |
| 8358 | | | and small cell neuroendocrine carcinoma |
| TCGA-BR- | 4.503 | STAD | Mixed gastric adenocarcinoma and small |
| A4CR | | | cell neuroendocrine carcinoma |
| TCGA-HU- | 4.325 | STAD | Moderately differentiated gastric adenocarcinoma |
| A4GD | | | with focal neuroendocrine differentiation |
| TCGA-HD- | 4.323 | HNSC | Mixed poorly differentiated head and neck squamous |
| A634 | | | carcinoma and small cell neuroendocrine carcinoma |
| TCGA-37- | 4.108 | LUSC | Neuroendocrine tumor of the lung (atypical |
| 4129 | | | carcinoid tumor) |
| TCGA-55- | 4.023 | LUAD | Mixed poorly differentiated lung adenocarcinoma |
| 8620 | | | and small cell neuroendocrine carcinoma |
| TCGA-VS- | 3.965 | CESC | Mixed poorly differentiated uterine cervical squamous |
| A9UP | | | carcinoma and small cell neuroendocrine carcinoma |
| TCGA-VS- | 3.899 | CESC | Mixed poorly differentiated uterine cervical squamous |
| A9UL | | | carcinoma and small cell neuroendocrine carcinoma |
| TCGA-D6- | 3.826 | HNSC | Mixed poorly differentiated head and neck squamous |
| A6EP | | | carcinoma and small cell neuroendocrine carcinoma |
| TCGA-LN- | 3.802 | ESCA | Mixed esophageal squamous carcinoma and small |
| A9FO | | | cell neuroendocrine carcinoma |
| TCGA-2H- | 3.477 | ESCA | NI * |
| A9GG | | | |
| TCGA-FE- | 3.442 | THCA | NI * |
| A239 | | | |
| TCGA-EL- | 3.365 | THCA | Papillary thyroid carcinoma with focal area |
| A4K6 | | | of neuroendocrine differentiation |
| TCGA-56- | 3.253 | LUSC | Mixed poorly differentiated lung squamous carcinoma |
| 7223 | | | and small cell neuroendocrine carcinoma |

| | Evidence | | | | | | |
|---|---|---|---|---|---|---|---|
| | of | | | | | | |
| | SCN- | | | | | | |
| | associatecd | | | | | | |
| | TP53 or | | | | | | |
| | RB1 | | | | | | |
| | genomic | TP53 | | | RB1 | | |
| Sample | alterations | mut | CNA | summary | mut | CNA | summary |
| TCGA-A6- | Yes | mut | −1 | LOF | 0 | 1 | |
| 5656 | | | | | | | |
| TCGA-XQ- | Yes | nonmut | −1 | LOF | 0 | −1 | LOF |
| A8TA | | | | | | | |
| TCGA-86- | Yes | NA | −1 | LOF | NA | −1 | LOF |
| 8358 | | | | | | | |
| TCGA-BR- | Yes | mut | 0 | LOF | 1 | 1 | LOF |
| A4CR | | | | | | | |
| TCGA-HU- | Yes | nonmut | −1 | LOF | 1 | 1 | LOF |
| A4GD | | | | | | | |
| TCGA-HD- | No | nonmut | 0 | | 0 | 0 | |
| A634 | | | | | | | |
| TCGA-37- | Yes | NA | 0 | | NA | −1 | LOF |
| 4129 | | | | | | | |
| TCGA-55- | Yes | NA | −2 | LOF | NA | −2 | LOF |
| 8620 | | | | | | | |
| TCGA-VS- | Yes | nonmut | −1 | LOF | 0 | −2 | LOF |
| A9UP | | | | | | | |
| TCGA-VS- | Yes | nonmut | 1 | | 0 | −1 | LOF |
| A9UL | | | | | | | |
| TCGA-D6- | Yes | mut | 2 | LOF | 0 | −1 | LOF |
| A6EP | | | | | | | |
| TCGA-LN- | Yes | nonmut | 0 | | 1 | −1 | LOF |
| A9FO | | | | | | | |
| TCGA-2H- | Yes | nonmut | NA | | 1 | NA | LOF |
| A9GG | | | | | | | |
| TCGA-FE- | Yes | NA | 0 | | NA | −1 | LOF |
| A239 | | | | | | | |
| TCGA-EL- | No | nonmut | 1 | | 0 | 0 | |
| A4K6 | | | | | | | |
| TCGA-56- | Yes | NA | −1 | LOF | NA | 1 | |
| 7223 | | | | | | | |

* NI = no small cell neuroendocrine carcinoma region identified.
Note
NI cases may be explained by tumors with focal SCN, and pathology calls being performed on tissue (paraffin) not adjacent to tissue used for sequencing (frozen).
Key: For CNA: −2 = deep deletion, −1 = deletion, 0 = unaltered, 1 = amplified, 2 = strong amplification; For mutation: mut = mutated, nonmut = not mutated, NA = not available; LOF: potential loss of function = mutation or CNA loss

TABLE S5-B
BRCA Pathology Call Statistics

| | “SCN | | | |
|---|---|---|---|---|
| | minus | | | |
| | proliferation” | | | |
| | score | | | SCN |
| BRCA | (PC1; | PAM | | histology |
| Sample | x-axis) | 50 | Pathology Call | features |
| TCGA-AC- | 7.3 | Basal | Mixed invasive ductal carcinoma and | |
| A2QH | | | small cell neuroendocrine carcinoma | |
| TCGA-A7- | 7.0 | Basal | Mixed invasive ductal carcinoma and | |
| A13D | | | small cell neuroendocrine carcinoma | |
| TCGA-A1- | 6.0 | Basal | Mixed large cell neuroendocrine carcinoma | |
| A0SK | | | and small cell neuroendocrine carcinoma | |
| TCGA-D8- | 5.2 | LumB | Mixed invasive ductal carcinoma and | |
| A13Y | | | small cell neuroendocrine carcinoma | |
| TCGA-BH- | 4.0 | LumA | Small cell neuroendocrine carcinoma | |
| A0HL | | | | |
| TCGA-E9- | 3.8 | LumA | Mixed invasive ductal carcinoma and | |
| A245 | | | small cell neuroendocrine carcinoma | |
| TCGA-C8- | 3.7 | LumB | Mixed invasive ductal carcinoma and | |
| A27A | | | small cell neuroendocrine carcinoma | |
| TCGA-A7- | 3.7 | LumA | Mixed intracystic papillary carcinoma | |
| A0DC | | | and small cell neuroendocrine carcinoma | |
| TCGA-A7- | 3.6 | Basal | Mixed invasive ductal carcinoma and | |
| A26I | | | small cell neuroendocrine carcinoma | |
| TCGA-A7- | 3.5 | LumA | Mixed invasive ductal carcinoma and | |
| A26E | | | small cell neuroendocrine carcinoma | |
| TCGA-B6- | 3.2 | LumB | Small cell neuroendocrine carcinoma | |
| A1KC | | | | |
| TCGA-E2- | 3.0 | LumB | Mixed invasive ductal carcinoma and | |
| A15S | | | small cell neuroendocrine carcinoma | |
| TCGA-AC- | 2.7 | LumA | Invasive ductal carcinoma | Negative |
| A3OD | | | | |
| TCGA-AR- | 2.5 | Basal | Small cell neuroendocrine carcinoma | |
| A2LR | | | | |
| TCGA-EW- | 2.3 | LumB | Invasive ductal carcinoma | Negative |
| A1OX | | | | |
| TCGA-D8- | 2.1 | LumA | Invasive ductal carcinoma | Negative |
| A1XD | | | | |
| TCGA-A7- | 2.0 | LumA | Invasive ductal carcinoma with | Negative |
| A3RF | | | abundant mucin (mucinous carcinoma) | |
| TCGA-A7- | 1.9 | LumA | Mixed invasive ductal carcinoma and | |
| A3J0 | | | small cell neuroendocrine carcinoma | |
| TCGA-E2- | 1.4 | LumA | Invasive ductal carcinoma | Negative |
| A14T | | | | |
| TCGA-A8- | 1.1 | Basal | Invasive ductal carcinoma | Negative |
| A07C | | | | |
| TCGA-GM- | 0.2 | LumA | Invasive ductal carcinoma | Negative |
| A2DA | | | | |
| TCGA-AO- | 0.1 | Basal | Invasive ductal carcinoma | Negative |
| A1KR | | | | |
| TCGA-3C- | −0.1 | LumA | Invasive ductal carcinoma | Negative |
| AALK | | | | |
| TCGA-E2- | −0.3 | LumA | Invasive ductal carcinoma with | Negative |
| A1IK | | | abundant mucin (mucinous carcinoma) | |
| TCGA-A2- | −0.7 | Normal | Invasive ductal carcinoma | Negative |
| A0CL | | | | |
| TCGA-LD- | −1.9 | Normal | Invasive ductal carcinoma | Negative |
| A9QF | | | | |
| TCGA-C8- | −1.9 | Her2 | Invasive ductal carcinoma | Negative |
| A26X | | | | |
| TCGA-D8- | −1.9 | Basal | Invasive ductal carcinoma | Negative |
| A1JK | | | | |
| TCGA-BH- | −1.9 | LumA | Invasive ductal carcinoma | Negative |
| A0B6 | | | | |
| TCGA-OL- | −2.0 | Her2 | Invasive ductal carcinoma | Negative |
| A66P | | | | |
| TCGA-AR- | −2.0 | Normal | Invasive ductal carcinoma | Negative |
| A2LH | | | | |
| TCGA-AC- | −2.0 | Basal | Invasive ductal carcinoma | Negative |
| A2QJ | | | | |
| TCGA-A8- | −2.1 | Normal | Invasive ductal carcinoma | Negative |
| A08H | | | | |
| TCGA-E9- | −2.1 | Basal | Invasive ductal carcinoma | Negative |
| A1ND | | | | |
| TCGA-OL- | −2.2 | Normal | Invasive ductal carcinoma | Negative |
| A5RY | | | | |
| TCGA-AR- | −2.4 | Basal | Invasive ductal carcinoma | Negative |
| A5QQ | | | | |
| TCGA-EW- | −2.4 | Basal | Invasive ductal carcinoma | Negative |
| A1PB | | | | |
| TCGA-AO- | −2.7 | Normal | Invasive ductal carcinoma | Negative |
| A03U | | | | |
| | | | Kolmogorov Smirnov enrichment p-value = | 9 × 10\hat{ }−7 |

| | SCN marker gene expression levels | | | | | | | |
|---|---|---|---|---|---|---|---|---|
| BRCA | normalized counts | | | | z-scores | | | |
| Sample | CGHA | SYP | NCAM1 | TTF1 | CGHA | SYP | NCAM1 | TTF1 |
| TCGA-AC- | 2.0 | 4.6 | 13.2 | 10.3 | 0.0 | −1.2 | 3.9 | 2.3 |
| A2QH | | | | | | | | |
| TCGA-A7- | 5.4 | 7.1 | 9.9 | 10.5 | 1.2 | 0.3 | 2.1 | 2.9 |
| A13D | | | | | | | | |
| TCGA-A1- | 10.5 | 10.9 | 13.0 | 9.9 | 3.1 | 2.7 | 3.8 | 1.4 |
| A0SK | | | | | | | | |
| TCGA-D8- | 17.0 | 11.0 | 8.2 | 9.4 | 5.5 | 2.8 | 1.1 | 0.3 |
| A13Y | | | | | | | | |
| TCGA-BH- | 14.9 | 12.0 | 4.8 | 9.2 | 4.7 | 3.4 | −0.8 | −0.2 |
| A0HL | | | | | | | | |
| TCGA-E9- | 14.8 | 13.1 | 6.2 | 10.4 | 4.7 | 4.1 | 0.0 | 2.6 |
| A245 | | | | | | | | |
| TCGA-C8- | 16.9 | 11.4 | 9.1 | 10.4 | 5.4 | 3.0 | 1.6 | 2.5 |
| A27A | | | | | | | | |
| TCGA-A7- | 3.9 | 7.9 | 5.2 | 9.5 | 0.7 | 0.8 | −0.6 | 0.5 |
| A0DC | | | | | | | | |
| TCGA-A7- | 3.8 | 7.4 | 9.0 | 9.4 | 0.6 | 0.6 | 1.5 | 0.4 |
| A26I | | | | | | | | |
| TCGA-A7- | 0.0 | 5.3 | 6.2 | 9.8 | −0.8 | −0.7 | −0.1 | 1.2 |
| A26E | | | | | | | | |
| TCGA-B6- | 15.3 | 12.3 | 5.8 | 8.9 | 4.9 | 3.6 | −0.2 | −0.8 |
| A1KC | | | | | | | | |
| TCGA-E2- | 3.8 | 8.7 | 4.2 | 9.3 | 0.6 | 1.4 | −1.2 | 0.1 |
| A15S | | | | | | | | |
| TCGA-AC- | 3.2 | 6.9 | 7.7 | 10.6 | 0.4 | 0.2 | 0.8 | 3.0 |
| A3OD | | | | | | | | |
| TCGA-AR- | 3.8 | 4.9 | 10.5 | 9.5 | 0.6 | −1.0 | 2.4 | 0.5 |
| A2LR | | | | | | | | |
| TCGA-EW- | 3.3 | 9.1 | 2.3 | 8.9 | 0.5 | 1.6 | −2.2 | −0.8 |
| A1OX | | | | | | | | |
| TCGA-D8- | 0.0 | 6.3 | 7.4 | 8.4 | −0.8 | −0.2 | 0.6 | −2.0 |
| A1XD | | | | | | | | |
| TCGA-A7- | 17.6 | 13.4 | 6.4 | 8.9 | 5.7 | 4.2 | 0.1 | −0.9 |
| A3RF | | | | | | | | |
| TCGA-A7- | 21.3 | 12.7 | 12.0 | 10.0 | 7.1 | 3.8 | 3.2 | 1.6 |
| A3J0 | | | | | | | | |
| TCGA-E2- | 16.0 | 13.2 | 5.3 | 9.5 | 5.1 | 4.1 | −0.6 | 0.6 |
| A14T | | | | | | | | |
| TCGA-A8- | 0.0 | 4.9 | 8.1 | 11.1 | −0.8 | −1.0 | 1.1 | 4.2 |
| A07C | | | | | | | | |
| TCGA-GM- | 1.4 | 6.0 | 6.9 | 8.9 | −0.3 | −0.3 | 0.4 | −0.9 |
| A2DA | | | | | | | | |
| TCGA-AO- | 0.0 | 5.6 | 5.5 | 9.0 | −0.8 | −0.6 | −0.4 | −0.7 |
| A1KR | | | | | | | | |
| TCGA-3C- | 2.4 | 6.1 | 13.0 | 9.2 | 0.1 | −0.3 | 3.8 | −0.2 |
| AALK | | | | | | | | |
| TCGA-E2- | 12.8 | 12.2 | 10.5 | 8.7 | 3.9 | 3.5 | 2.4 | −1.3 |
| A1IK | | | | | | | | |
| TCGA-A2- | 14.1 | 7.2 | 6.9 | 9.1 | 4.4 | 0.4 | 0.4 | −0.5 |
| A0CL | | | | | | | | |
| TCGA-LD- | 2.5 | 6.5 | 6.1 | 9.0 | 0.2 | 0.0 | −0.1 | −0.6 |
| A9QF | | | | | | | | |
| TCGA-C8- | 0.0 | 5.3 | 6.0 | 8.7 | −0.8 | −0.7 | −0.1 | −1.3 |
| A26X | | | | | | | | |
| TCGA-D8- | 0.0 | 5.4 | 5.0 | 9.3 | −0.8 | −0.7 | −0.7 | 0.1 |
| A1JK | | | | | | | | |
| TCGA-BH- | 2.6 | 6.1 | 6.9 | 8.4 | 0.2 | −0.3 | 0.4 | −2.0 |
| A0B6 | | | | | | | | |
| TCGA-OL- | 3.5 | 6.3 | 6.2 | 8.7 | 0.5 | −0.1 | 0.0 | −1.4 |
| A66P | | | | | | | | |
| TCGA-AR- | 3.2 | 5.2 | 8.0 | 9.3 | 0.4 | −0.8 | 1.0 | 0.1 |
| A2LH | | | | | | | | |
| TCGA-AC- | 6.0 | 6.8 | 5.4 | 7.9 | 1.4 | 0.2 | −0.5 | −3.2 |
| A2QJ | | | | | | | | |
| TCGA-A8- | 5.4 | 6.0 | 6.9 | 9.1 | 1.2 | −0.4 | 0.4 | −0.5 |
| A08H | | | | | | | | |
| TCGA-E9- | 1.4 | 5.1 | 5.3 | 9.4 | −0.3 | −0.9 | −0.5 | 0.3 |
| A1ND | | | | | | | | |
| TCGA-OL- | 2.8 | 4.9 | 6.4 | 9.4 | 0.3 | −1.0 | 0.1 | 0.2 |
| A5RY | | | | | | | | |
| TCGA-AR- | 3.0 | 4.5 | 9.8 | 9.3 | 0.3 | −1.3 | 2.0 | 0.0 |
| A5QQ | | | | | | | | |
| TCGA-EW- | 1.9 | 6.2 | 5.9 | 8.7 | −0.1 | −0.2 | −0.2 | −1.3 |
| A1PB | | | | | | | | |
| TCGA-AO- | 0.0 | 5.1 | 9.7 | 9.3 | −0.8 | −0.9 | 2.0 | 0.1 |
| A03U | | | | | | | | |

TABLE S5-C
BRCA Mutation Analysis

| | | | | TP53 |
|---|---|---|---|---|
| | | SCN Score | | pathway |
| | | Prolif | Sub | TP53 |
| Sample | Pathology Call | Removed | type | mutation |
| TCGA.AC. | Mixed invasive ductal carcinoma and | 7.300 | Basal | mut |
| A2QH.01 | small cell neuroendocrine carcinoma | | | |
| TCGA.A7. | Mixed inv asiv e ductal carcinoma and | 7.001 | Basal | mut |
| A13D.01 | small cell neuroendocrine carcinoma | | | |
| TCGA.A1. | Mixed large cell neuroendocrine carcinoma | 6.039 | Basal | mut |
| A0SK.01 | and small cell neuroendocrine carcinoma | | | |
| TCGA.D8. | Mixed invasive ductal carcinoma and | 5.202 | LumB | nonmut |
| A13Y.01 | small cell neuroendocrine carcinoma | | | |
| TCGA.E9. | Mixed invasive ductal carcinoma and | 3.846 | LumA | nonmut |
| A245.01 | small cell neuroendocrine carcinoma | | | |
| TCGA.C8. | Mixed invasive ductal carcinoma and | 3.737 | LumB | mut |
| A27A.01 | small cell neuroendocrine carcinoma | | | |
| TCGA.A7. | Mixed intracystic papillary carcinoma | 3.685 | LumA | nonmut |
| A0DC.01 | andsmall cell neuroendocrine carcinoma | | | |
| TCGA.A7. | Mixed invasive ductal carcinoma and | 3.628 | Basal | mut |
| A26I.01 | small cell neuroendocrine carcinoma | | | |
| TCGA.A7. | Mixed invasive ductal carcinoma and | 3.507 | LumA | nonmut |
| A26E.01 | small cell neuroendocrine carcinoma | | | |
| TCGA.B6. | Small cell neuroendocrine carcinoma | 3.196 | LumB | nonmut |
| A1KC.01 | | | | |

| | TP53 pathway | | | RB1 gene | | TP53 | |
|---|---|---|---|---|---|---|---|
| | TP53 | MDM1/2 | MDM4 | RB1 | RB1 | Pathway | RB1 |
| Sample | CNA | CNA | CNA | mutation | CNA | Summary | Summary |
| TCGA.AC. | 1 | 0 | 0 | nonmut | −1 | TP53 mut | RB1 loss |
| A2QH.01 | | | | | | | |
| TCGA.A7. | 1 | −1 | 1 | nonmut | −1 | TP53 mut, | RB1 loss |
| A13D.01 | | | | | | MDM4 amp | |
| TCGA.A1. | −1 | 0 | −1 | nonmut | −2 | TP53 mut, | RB1 deep |
| A0SK.01 | | | | | | TP53 loss | deletion |
| TCGA.D8. | −1 | 1 | 0 | nonmut | −1 | TP53 loss, | RB1 loss |
| A13Y.01 | | | | | | MDM1/2 amp | |
| TCGA.E9. | 0 | 0 | 1 | nonmut | 0 | MDM4 amp | |
| A245.01 | | | | | | | |
| TCGA.C8. | −1 | −1 | 0 | nonmut | 0 | TP53 mut, | |
| A27A.01 | | | | | | TP53 loss | |
| TCGA.A7. | 1 | 1 | −1 | nonmut | −1 | MDM1/2 amp | RB1 loss |
| A0DC.01 | | | | | | | |
| TCGA.A7. | −1 | −1 | 1 | nonmut | −1 | TP53 mut, | RB1 loss |
| A26I.01 | | | | | | TP53 loss, | |
| | | | | | | MDM4 amp | |
| TCGA.A7. | −1 | 0 | 1 | nonmut | −1 | TP53 loss, | RB1 loss |
| A26E.01 | | | | | | MDM4 amp | |
| TCGA.B6. | 1 | 1 | 1 | nonmut | −1 | MDM1/2 amp, | RB1 loss |
| A1KC.01 | | | | | | MDM4 amp | |

Key: For CNA: −2 = deep deletion, −1 = deletion, 0 = unaltered, 1 = amplified, 2 = strong amplification; For mutation: mut = mutated, nonmut = not mutated.

### Example 2: Methods for Diagnosing the SCN Phenotype

A. Results

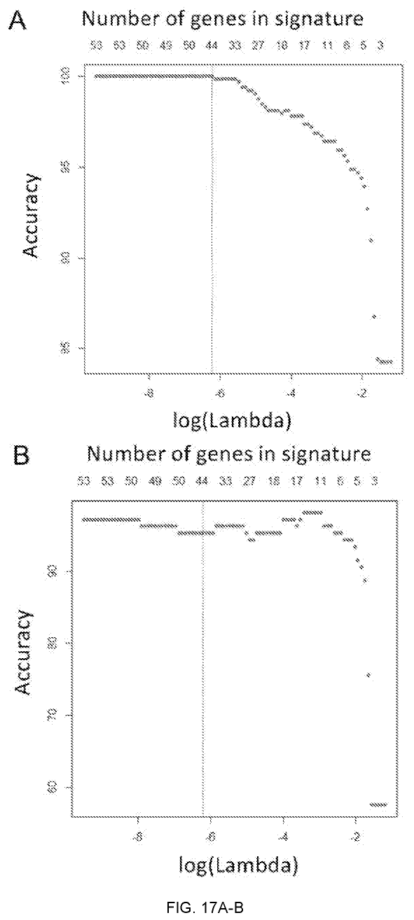

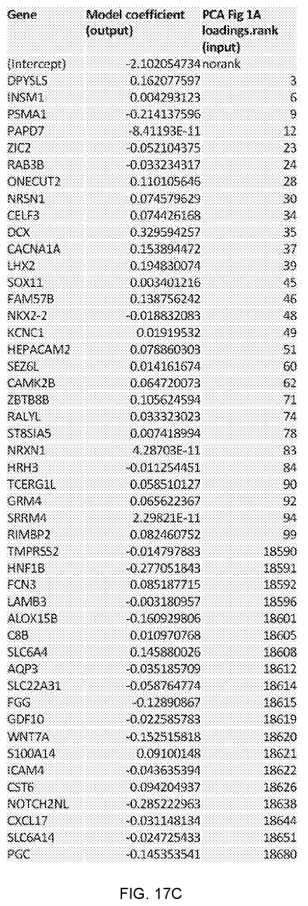

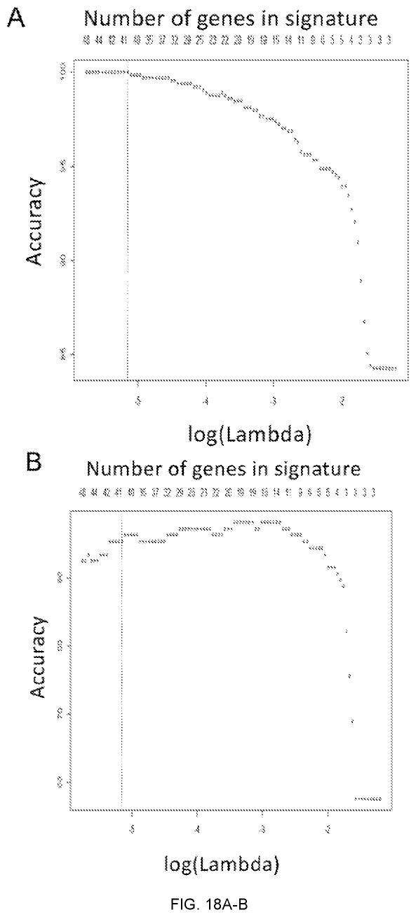

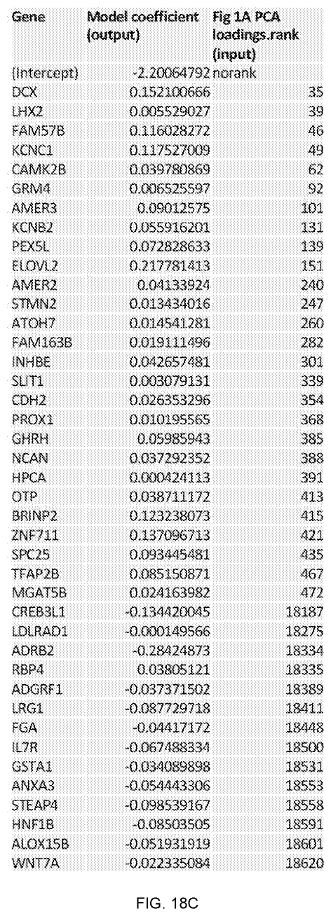

The inventors generated an algorithm to predict tumors and cell lines with SCN features based on the gene expression profiles of SCN tumors. As input gene sets the inventors used the top and bottom of the lists of SCN signature created from the PCA loadings of FIG. 1A. Training of a logistic regression model with LASSO (LRL) on the top and bottom 100 genes from the loadings was predictive of SCN cases in the original data (FIG. 17A), and of SCN cell lines in the CCLE lung test set (FIG. 17B), and resulted in a model of 47 genes (FIG. 17C). An additional predictive model was built starting with the top 500 and bottom 500 genes and resulted in a model of 41 genes (FIG. 18A-C). This data highlights that different sets of final genes for the top N lists can be similarly informative. This result demonstrates the ability for the inventors' signatures to be of diagnostic use in the clinic to predict cases of SCN cancer, which suffer from poor overall survival (FIG. 3B).

B. Methods

Prediction of samples with SCN features using a logistic regression model with LASSO (LRL). Logistic Regression with LASSO (LRL) was used to select subsets of genes from list of top N/bottom N genes (e.g., N=100, 500) from the ranked loadings of the SCN PCA framework in FIG. 1A to determine a compact signature predictive of samples with an SCN phenotype. Samples included in the model were LUAD, SCLC, NEPC, CRPC, and the pan-cancer SCN positive cases confirmed by pathology (BRCA, CESC, COADREAD, ESCA, HNSC, LUAD, LUSC, PRAD, STAD, THCA; Table S5). The LASSO method allows the selection of a subset of genes from these gene lists, to create a compact signature useful for assay development. LRL was used to assign a probability (on a scale from 0 to 1) to each sample belonging to the Small Cell Neuroendocrine (SCN, 1) or non-Small Cell Neuroendocrine (non-SCN, 0). Cases above 0.5 were assigned to the SCN class, while those below to the Non-SCN class. Algorithm implementation was performed using R with the glmnet package.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Alanee, S., Moore, A., Nutt, M., Holland, B., Dynda, D., El-Zawahry, A. & McVary, K. T. (2015). Contemporary Incidence and Mortality Rates of Neuroendocrine Prostate Cancer. Anticancer Res. 35, 4145-4150.
2. Alvarez, M. J., Subramaniam, P. S., Tang, L. H., Grunn, A., Aburi, M., Rieckhof, G., Komissarova, E. V., Hagan, E. A., Bodei, L., et al. (2018). A precision oncology approach to the pharmacological targeting of mechanistic dependencies in neuroendocrine tumors. Nat. Genet. 50, 979-989.
3. Alvarez, M. J., Shen, Y., Giorgi, F. M., Lachmann, A., Ding, B. B., Ye, B. H., and Califano, A. (2016). Functional characterization of somatic mutations in cancer using network-based inference of protein activity. Nat. Genet. 48, 838-847.
4. Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
5. Barretina, J., Caponigro, G., Stransky, N., Venkatesan, K., Margolin, A. A., Kim, S., Wilson, C. J., Lehár, J., Kryukov, G. V., Sonkin, D., et al. (2012). The Cancer Cell Line Encyclopedia enables predictive modelling of anticancer drug sensitivity. Nature 483, 603-307.
6. Beltran, H., Prandi, D., Mosquera, J. M., Benelli, M., Puca, L., Cyrta, J., Marotz, C., Giannopoulou, E., Chakravarthi, B. V. S. K., et al. (2016). Divergent clonal evolution of castration-resistant neuroendocrine prostate cancer. Nat. Med. 22, 298-305.
7. Bollard, J., Miguela, V., Ruiz de Galarreta, M., Venkatesh, A., Bian, C. B., Roberto, M. P., Tovar, V., Sia, D., Molina-Sanchez, P., et al. (2017). Palbociclib (PD-0332991), a selective CDK4/6 inhibitor, restricts tumour growth in preclinical models of hepatocellular carcinoma. Gut 66, 1286-1296.
8. Borromeo, M. D., Savage, T. K., Kollipara, R. K., He, M., Augustyn, A., Osborne, J. K., Girard, L., Minna, J. D., Gazdar, A. F., et al. (2016). ASCL1 and NEUROD1 Reveal Heterogeneity in Pulmonary Neuroendocrine Tumors and Regulate Distinct Genetic Programs. Cell Rep. 16, 1259-1272.
9. Carlson, M. (2018). org.Hs.eg.db: Genome wide annotation for Human. R package version 3.6.0.
10. Cayrol, F., Praditsuktavorn, P., Fernando, T. M., Kwiatkowski, N., Marullo, R., Calvo-Vidal, M. N., Phillip, J., Pera, B., Yang, S. N., et al. (2017). THZ1 targeting CDK7 suppresses STAT transcriptional activity and sensitizes T-cell lymphomas to BCL2 inhibitors. Nat. Commun. 8, 14290.
11. Chen, F., Zhang, Y., Gibbons, D. L., Deneen, B., Kwiatkowski, D. J., Ittmann, M. & Creighton, C. J. (2018). Pan-Cancer Molecular Classes Transcending Tumor Lineage Across 32 Cancer Types, Multiple Data Platforms, and over 10,000 Cases. Clin. Cancer Res.
12. Chipumuro, E., Marco, E., Christensen, C. L., Kwiatkowski, N., Zhang, T., Hatheway, C. M., Abraham, B. J., Sharma, B., Yeung, C., et al. (2014). CDK7 Inhibition Suppresses Super-Enhancer-Linked Oncogenic Transcription in MYCN-Driven Cancer. Cell 159, 1126-1139.
13. Cho, S. Y., Choi, M., Ban, H.-J., Lee, C. H., Park, S., Kim, H., Kim, Y.-S., Lee, Y. S. & Lee, J.-Y. (2016). Cervical small cell neuroendocrine tumor mutation profiles via whole exome sequencing. Oncotarget 8, 8095-8104.
14. Christensen, C. L., Kwiatkowski, N., Abraham, B. J., Carretero, J., Al-Shahrour, F., Zhang, T., Chipumuro, E., Herter-Sprie, G. S., Akbay, E. A., et al. (2014). Targeting Transcriptional Addictions in Small Cell Lung Cancer with a Covalent CDK7 Inhibitor. Cancer Cell 26, 909-922.
15. CLCGP et al. (2013). A Genomics-Based Classification of Human Lung Tumors. Sci. Transl. Med. 5, 209ra153.
16. Cole, J., Guiot, M.-C., Gravel, M., Bernier, C., Shore, G. C. & Roulston, A. (2017). Novel NAPRT specific antibody identifies small cell lung cancer and neuronal cancers as promising clinical indications for a NAMPT inhibitor/niacin co-administration strategy. Oncotarget 8.
17. Condorelli, R., Spring, L., O'Shaughnessy, J., Lacroix, L., Bailleux, C., Scott, V., Dubois, J., Nagy, R. J., Lanman, R. B., et al. (2018). Polyclonal RB1 mutations and acquired resistance to CDK 4/6 inhibitors in patients with metastatic breast cancer. Ann. Oncol. Off. J. Eur. Soc. Med. Oncol. 29, 640-645.
18. Cyrenne, B. M., Lewis, J. M., Weed, J. G., Carlson, K. R., Mirza, F. N., Foss, F. M. & Girardi, M. (2017). Synergy of BCL2 and histone deacetylase inhibition against leukemic cells from cutaneous T-cell lymphoma patients. Blood 130, 2073-2083.
19. Dardenne, E., Beltran, H., Benelli, M., Gayvert, K., Berger, A., Puca, L., Cyrta, J., Sboner, A., Noorzad, Z., et al. (2016). N-Myc Induces an EZH2-Mediated Transcriptional Program Driving Neuroendocrine Prostate Cancer. Cancer Cell 30, 563-577.
20. De Las Casas, L. E., Gokden, M., Mukunyadzi, P., White, P., Baker, S. J., Hermonat, P. L., You, H., Korourian, S., Malak, S. F., et al. (2004). A morphologic and statistical comparative study of small-cell carcinoma and non-Hodgkin's lymphoma in fine-needle aspiration biopsy material from lymph nodes. Diagn. Cytopathol. 31, 229-234.
21. Doerr, F., George, J., Schmitt, A., Beleggia, F., Rehkamper, T., Hermann, S., Walter, V., Weber, J.-P., Thomas, R. K., et al. (2017). Targeting a non-oncogene addiction to the ATR/CHK1 axis for the treatment of small cell lung cancer. Sci. Rep. 7, 15511.
22. Feng, N., Yin, Y., He, Y. & Huang, J. (2017). Alternative Splicing Provides a Novel Molecular Mechanism for Prostatic Small-cell Neuroendocrine Carcinoma. Eur. Urol. 71, 79-80.
23. George, J., Lim, J. S., Jang, S. J., Cun, Y., Ozretić, L., Kong, G., Leenders, F., Lu, X., Fernandez-Cuesta, L., et al. (2015). Comprehensive genomic profiles of small cell lung cancer. Nature 524, 47-53.
24. Goethe, E., Carter, B. Z., Rao, G. & Pemmaraju, N. (2018). Glioblastoma and acute myeloid leukemia: malignancies with striking similarities. J. Neurooncol. 136, 223-231.
25. Graham, N. A., Minasyan, A., Lomova, A., Cass, A., Balanis, N. G., Friedman, M., Chan, S., Zhao, S., Delgado, A., et al. (2017). Recurrent patterns of DNA copy number alterations in tumors reflect metabolic selection pressures. Mol. Syst. Biol. 13, 914.
26. Inno, A., Bogina, G., Turazza, M., Bortesi, L., Duranti, S., Massocco, A., Zamboni, G., Carbognin, G., Alongi, F., et al. (2016). Neuroendocrine Carcinoma of the Breast: Current Evidence and Future Perspectives. The Oncologist 21, 28-32.
27. Iorio, F., Knijnenburg, T. A., Vis, D. J., Bignell, G. R., Menden, M. P., Schubert, M., Aben, N., Gonsalves, E., Barthorpe, S., et al. (2016). A Landscape of Pharmacogenomic Interactions in Cancer. Cell 166, 740-754.
28. Kim, J., Jin, H., Zhao, J. C., Yang, Y. A., Li, Y., Yang, X., Dong, X. & Yu, J. (2017). FOXA1 inhibits prostate cancer neuroendocrine differentiation. Oncogene 36, 4072-4080.
29. Klimstra, D. S., Beltran, H., Lilenbaum, R. & Bergsland, E. (2015). The Spectrum of Neuroendocrine Tumors: Histologic Classification, Unique Features and Areas of Overlap. Am. Soc. Clin. Oncol. Educ. Book 35, 92-103.
30. Ku, S. Y., Rosario, S., Wang, Y., Mu, P., Seshadri, M., Goodrich, Z. W., Goodrich, M. M., Labbé, D. P., Gomez, E. C., et al. (2017). Rb1 and Trp53 cooperate to suppress prostate cancer lineage plasticity, metastasis, and antiandrogen resistance. Science 355, 78-83.
31. Kuhn, M. C. from J. W., Steve Weston, Andre Williams, Chris Keefer, Allan Engelhardt, Tony Cooper, Zachary Mayer, Brenton Kenkel, the R. Core Team, Michael Benesty, Reynald Lescarbeau, Andrew Ziem, Luca Scrucca, Yuan Tang and Can Candan. (2016). caret: Classification and Regression.
32. Lachmann, A., Giorgi, F. M., Lopez, G., and Califano, A. (2016a). ARACNe-AP: gene network reverse engineering through adaptive partitioning inference of mutual information. Bioinformatics 32, 2233-2235.
33. Lachmann, A., Giorgi, F. M., Lopez, G., and Califano, A. (2016b). ARACNe-AP: gene network reverse engineering through adaptive partitioning inference of mutual information. Bioinforma. Oxf. Engl. 32, 2233-2235.
34. Lapenna, S. & Giordano, A. (2009). Cell cycle kinases as therapeutic targets for cancer. Nat. Rev. Drug Discov. 8, 547-566.
35. Lee, J. K., Phillips, J. W., Smith, B. A., Park, J. W., Stoyanova, T., McCaffrey, E. F., Baertsch, R., Sokolov, A., Meyerowitz, J. G., et al. (2016). N-Myc Drives Neuroendocrine Prostate Cancer Initiated from Human Prostate Epithelial Cells. Cancer Cell 29, 536-547.
36. Lee, J.-K., Lee, J., Kim, S., Kim, S., Youk, J., Park, S., An, Y., Keam, B., Kim, D.-W., et al. (2017). Clonal History and Genetic Predictors of Transformation Into Small-Cell Carcinomas From Lung Adenocarcinomas. J. Clin. Oncol. Off. J. Am. Soc. Clin. Oncol. 35, 3065-3074.
37. Li, J., Zhao, W., Akbani, R., Liu, W., Ju, Z., Ling, S., Vellano, C. P., Roebuck, P., Yu, Q., et al. (2017). Characterization of Human Cancer Cell Lines by Reverse-phase Protein Arrays. Cancer Cell 31, 225-239.
38. Lim, J. S., Ibaseta, A., Fischer, M. M., Cancilla, B., O'Young, G., Cristea, S., Luca, V. C., Yang, D., Jahchan, N. S., et al. (2017). Intratumoural heterogeneity generated by Notch signalling promotes small-cell lung cancer. Nature 545, 360-364.
39. Lochmann, T. L., Floros, K. V., Naseri, M., Powell, K. M., Cook, W., March, R. J., Stein, G. T., Greninger, P., Maves, Y. K., et al. (2018). Venetoclax Is Effective in Small-Cell Lung Cancers with High BCL-2 Expression. Clin. Cancer Res. 24, 360-369.
40. Lucas, D. R., Bentley, G., Dan, M. E., Tabaczka, P., Poulik, J. M. & Mott, M. P. (2001). Ewing sarcoma vs lymphoblastic lymphoma. A comparative immunohistochemical study. Am. J. Clin. Pathol. 115, 11-17.
41. Malta, T. M., Sokolov, A., Gentles, A. J., Burzykowski, T., Poisson, L., Weinstein, J. N., Kamińska, B., Huelsken, J., Omberg, L., et al. (2018). Machine Learning Identifies Stemness Features Associated with Oncogenic Dedifferentiation. Cell 173, 338-354.e15.
42. Marcoux, N., Gettinger, S. N., O'Kane, G., Arbour, K. C., Neal, J. W., Husain, H., Evans, T. L., Brahmer, J. R., Muzikansky, A., et al. (2018). EGFR—Mutant Adenocarcinomas That Transform to Small-Cell Lung Cancer and Other Neuroendocrine Carcinomas: Clinical Outcomes. J. Clin. Oncol. JCO.18.01585.
43. Mayrhofer, M., Viklund, B., and Isaksson, A. (2016). Rawcopy: Improved copy number analysis with Affymetrix arrays. Sci. Rep. 6.
44. van Meerbeeck, J. P., Fennell, D. A. & De Ruysscher, D. K. M. (2011). Small-cell lung cancer. Lancet Lond. Engl. 378, 1741-1755.
45. Mermel, C. H., Schumacher, S. E., Hill, B., Meyerson, M. L., Beroukhim, R., and Getz, G. (2011). GISTIC2.0 facilitates sensitive and confident localization of the targets of focal somatic copy-number alteration in human cancers. Genome Biol. 12, R41.
46. Mohammad, H. P., Smitheman, K. N., Kamat, C. D., Soong, D., Federowicz, K. E., Van Aller, G. S., Schneck, J. L., Carson, J. D., Liu, Y., Butticello, M., et al. (2015). A DNA Hypomethylation Signature Predicts Antitumor Activity of LSD1 Inhibitors in SCLC. Cancer Cell 28, 57-69.
47. Nadal, R., Schweizer, M., Kryvenko, 0. N., Epstein, J. I. & Eisenberger, M. A. (2014). Small cell carcinoma of the prostate. Nat. Rev. Urol. 11, 213-219.
48. Niederst, M. J., Sequist, L. V., Poirier, J. T., Mermel, C. H., Lockerman, E. L., Garcia, A. R., Katayama, R., Costa, C., Ross, K. N., et al. (2015). RB loss in resistant EGFR mutant lung adenocarcinomas that transform to small-cell lung cancer. Nat. Commun. 6, 6377.
49. Nilson, K. A., Guo, J., Turek, M. E., Brogie, J. E., Delaney, E., Luse, D. S. & Price, D. H. (2015). THZ1 Reveals Roles for Cdk7 in Co-transcriptional Capping and Pausing. Mol. Cell 59, 576-587.
50. Oberg, K., Modlin, I. M., De Herder, W., Pavel, M., Klimstra, D., Frilling, A., Metz, D. C., Heaney, A., Kwekkeboom, D., et al. (2015). Consensus on biomarkers for neuroendocrine tumour disease. Lancet Oncol. 16, e435-e446.
51. Oronsky, B., Ma, P. C., Morgensztern, D. & Carter, C. A. (2017). Nothing But NET: A Review of Neuroendocrine Tumors and Carcinomas. Neoplasia N. Y. N 19, 991-1002.
52. Ozdemirli, M., Fanburg-Smith, J. C., Hartmann, D. P., Shad, A. T., Lage, J. M., Magrath, I. T., Azumi, N., Harris, N. L., Cossman, J., et al. (1998). Precursor B-Lymphoblastic lymphoma presenting as a solitary bone tumor and mimicking Ewing's sarcoma: a report of four cases and review of the literature. Am. J. Surg. Pathol. 22, 795-804.
53. Park, J. W., Lee, J. K., Sheu, K. M., Wang, L., Balanis, N. G., Nguyen, K., Smith, B. A., Cheng, C., Tsai, B. L., et al. (2018). Reprogramming normal human epithelial tissues to a common, lethal neuroendocrine cancer lineage. Science 362, 91-95.
54. Park, K.-S., Liang, M.-C., Raiser, D. M., Zamponi, R., Roach, R. R., Curtis, S. J., Walton, Z., Schaffer, B. E., Roake, C. M., et al. (2011). Characterization of the cell of origin for small cell lung cancer. Cell Cycle Georget. Ill. 10, 2806-2815.
55. Pascual, G., Avgustinova, A., Mejetta, S., Martin, M., Castellanos, A., Attolini, C. S.-O., Berenguer, A., Prats, N., Toll, A., et al. (2017). Targeting metastasis-initiating cells through the fatty acid receptor CD36. Nature 541, 41-45.
56. Patro, R., Duggal, G., Love, M. I., Irizarry, R. A., and Kingsford, C. (2017). Salmon provides fast and bias-aware quantification of transcript expression. Nat. Methods 14, 417-419.
57. Pedersen, B. S., Eyring, K., De, S., Yang, I. V., and Schwartz, D. A. (2014). Fast and accurate alignment of long bisulfite-seq reads. ArXiv14011129 Q-Bio.
58. Plaisier, S. B., Taschereau, R., Wong, J. A. & Graeber, T. G. (2010). Rank-rank hypergeometric overlap: identification of statistically significant overlap between gene-expression signatures. Nucleic Acids Res. 38, e169.
59. Platta, C. S., Greenblatt, D. Y., Kunnimalaiyaan, M. & Chen, H. (2007). The HDAC Inhibitor Trichostatin A Inhibits Growth of Small Cell Lung Cancer Cells. J. Surg. Res. 142, 219-226.
60. Rickman, D. S., Beltran, H., Demichelis, F. & Rubin, M. A. (2017). Biology and evolution of poorly differentiated neuroendocrine tumors. Nat. Med. 23, 1-10.
61. Robertson, A. G., Kim, J., Al-Ahmadie, H., Bellmunt, J., Guo, G., Cherniack, A. D., Hinoue, T., Laird, P. W., Hoadley, K. A., et al. (2018). Comprehensive Molecular Characterization of Muscle-Invasive Bladder Cancer. Cell 174, 1033.
62. Robin, X., Turck, N., Hainard, A., Tiberti, N., Lisacek, F., Sanchez, J.-C., and Müller, M. (2011). pROC: an open-source package for R and S+ to analyze and compare ROC curves. BMC Bioinformatics 12, 77.
63. Robinson, D. R., Wu, Y.-M., Lonigro, R. J., Vats, P., Cobain, E., Everett, J., Cao, X., Rabban, E., Kumar-Sinha, C., et al. (2017). Integrative clinical genomics of metastatic cancer. Nature 548, 297-303.
64. Rohart, F., Gautier, B., Singh, A., and Lê Cao, K.-A. (2017). mixOmics: An R package for ‘omics feature selection and multiple data integration. PLOS Comput. Biol. 13, e1005752.
65. Rudin, C. M., Durinck, S., Stawiski, E. W., Poirier, J. T., Modrusan, Z., Shames, D. S., Bergbower, E. A., Guan, Y., Shin, J., et al. (2012). Comprehensive genomic analysis identifies SOX2 as a frequently amplified gene in small-cell lung cancer. Nat. Genet. 44, 1111-1116.
66. Seymour, J. F., Kipps, T. J., Eichhorst, B., Hillmen, P., D'Rozario, J., Assouline, S., Owen, C., Gerecitano, J., Robak, T., et al. (2018). Venetoclax-Rituximab in Relapsed or Refractory Chronic Lymphocytic Leukemia. N. Engl. J. Med. 378, 1107-1120.
67. Smith, B. A., Balanis, N. G., Nanjundiah, A., Sheu, K. M., Tsai, B. L., Zhang, Q., Park, J. W., Thompson, M., Huang, J., et al. (2018). A Human Adult Stem Cell Signature Marks Aggressive Variants across Epithelial Cancers. Cell Rep. 24, 3353-3366.e5.
68. Stacklies, W., Redestig, H., Scholz, M., Walther, D., and Selbig, J. (2007). pcaMethods—a bioconductor package providing PCA methods for incomplete data. Bioinforma. Oxf. Engl. 23, 1164-1167.
69. Subramanian, A., Tamayo, P., Mootha, V. K., Mukherjee, S., Ebert, B. L., Gillette, M. A., Paulovich, A., Pomeroy, S. L., Golub, T. R., and Lander, E. S. (2005). Gene set enrichment analysis: a knowledge-based approach for interpreting genome-wide expression profiles. Proc. Natl. Acad. Sci. 102, 15545-15550.
70. Sur, I. & Taipale, J. (2016). The role of enhancers in cancer. Nat. Rev. Cancer 16, 483-493.
71. Sutherland, K. D., Proost, N., Brouns, I., Adriaensen, D., Song, J.-Y. & Berns, A. (2011). Cell of Origin of Small Cell Lung Cancer: Inactivation of Trp53 and Rb1 in Distinct Cell Types of Adult Mouse Lung. Cancer Cell 19, 754-764.
72. Therneau, T. M., and Grambsch, P. M. (2000). Modeling Survival Data: Extending the Cox Model (Springer Science & Business Media).
73. Treviño, V., Martinez-Ledesma, E., and Tamez-Peña, J. (2017). Identification of outcome-related driver mutations in cancer using conditional co-occurrence distributions. Sci. Rep. 7, 43350.
74. Tsherniak, A., Vazquez, F., Montgomery, P. G., Weir, B. A., Kryukov, G., Cowley, G. S., Gill, S., Harrington, W. F., Pantel, S., et al. (2017). Defining a Cancer Dependency Map. Cell 170, 564-576.e16.
75. Tsoi, J., Robert, L., Paraiso, K., Galvan, C., Sheu, K. M., Lay, J., Wong, D. J. L., Atefi, M., Shirazi, R., et al. (2018). Multi-stage Differentiation Defines Melanoma Subtypes with Differential Vulnerability to Drug-Induced Iron-Dependent Oxidative Stress. Cancer Cell 33, 890-904.e5.
76. Verhaak, R. G. W., Hoadley, K. A., Purdom, E., Wang, V., Qi, Y., Wilkerson, M. D., Miller, C. R., Ding, L., Golub, T., et al. (2010). Integrated Genomic Analysis Identifies Clinically Relevant Subtypes of Glioblastoma Characterized by Abnormalities in PDGFRA, IDH1, EGFR, and NF1. Cancer Cell 17, 98-110.
77. Vivian, J., Rao, A. A., Nothaft, F. A., Ketchum, C., Armstrong, J., Novak, A., Pfeil, J., Narkizian, J., Deran, A. D., Musselman-Brown, A., et al. (2017). Toil enables reproducible, open source, big biomedical data analyses. Nat. Biotechnol. 35, 314-316.
78. Wang, C.-Y., Shahi, P., Huang, J. T. W., Phan, N. N., Sun, Z., Lin, Y.-C., Lai, M.-D. & Werb, Z. (2017). Systematic analysis of the achaete-scute complex-like gene signature in clinical cancer patients. Mol. Clin. Oncol. 6, 7-18.
79. Wang, J., Wei, B., Albarracin, C. T., Hu, J., Abraham, S. C. & Wu, Y. (2014). Invasive neuroendocrine carcinoma of the breast: a population-based study from the surveillance, epidemiology and end results (SEER) database. BMC Cancer 14, 147.
80. Watson, G. A., Ahmed, Y., Picardo, S., Chew, S., Cobbe, S., Mahony, C., Crotty, J., Wallis, F., Shelly, M. J., et al. (2018). Unusual Sites of High-Grade Neuroendocrine Carcinomas: A Case Series and Review of the Literature. Am. J. Case Rep. 19, 710-723.
81. Watson, M., Roulston, A., Belec, L., Billot, X., Marcellus, R., Bedard, D., Bernier, C., Branchaud, S., Chan, H., et al. (2009). The Small Molecule GMX1778 Is a Potent Inhibitor of NAD+ Biosynthesis: Strategy for Enhanced Therapy in Nicotinic Acid Phosphoribosyltransferase 1-Deficient Tumors. Mol. Cell. Biol. 29, 5872-5888.
82. Watson, P. A., Arora, V. K. & Sawyers, C. L. (2015). Emerging mechanisms of resistance to androgen receptor inhibitors in prostate cancer. Nat. Rev. Cancer 15, 701-711.
83. Wickham, H. (2016). ggplot2: Elegant Graphics for Data Analysis, 2016 (Springer-Verlag New York).
84. Wong, Y. N. S., Jack, R. H., Mak, V., Henrik, M. & Davies, E. A. (2009). The epidemiology and survival of extrapulmonary small cell carcinoma in South East England, 1970-2004. BMC Cancer 9.
85. Yachida, S., Vakiani, E., White, C. M., Zhong, Y., Saunders, T., Morgan, R., de Wilde, R. F., Maitra, A., Hicks, J., et al. (2012). Small Cell and Large Cell Neuroendocrine Carcinomas of the Pancreas are Genetically Similar and Distinct From Well-differentiated Pancreatic Neuroendocrine Tumors: Am. J. Surg. Pathol. 36, 173-184.
86. Yang, D., Denny, S. K., Greenside, P. G., Chaikovsky, A. C., Brady, J. J., Ouadah, Y., Granja, J. M., Jahchan, N. S., Lim, J. S., et al. (2018). Intertumoral Heterogeneity in SCLC Is Influenced by the Cell Type of Origin. Cancer Discov. 8, 1316-1331.
87. Zaykin, D. V. (2011). Optimally weighted Z-test is a powerful method for combining probabilities in meta-analysis. J. Evol. Biol. 24, 1836-1841.
88. Zhang, X., Coleman, I. M., Brown, L. G., True, L. D., Kollath, L., Lucas, J. M., Lam, H.-M., Dumpit, R., Corey, E., et al. (2015). SRRM4 Expression and the Loss of REST Activity May Promote the Emergence of the Neuroendocrine Phenotype in Castration-Resistant Prostate Cancer. Clin. Cancer Res. 21, 4698-4708.

What is claimed is:

1. A method comprising measuring the level of expression of one or more biomarkers from Tables 1-3 in a biological sample from a cancer patient.

2. The method of claim 1, wherein the patient has an epithelial or hematological cancer.

3. The method of claim 1 or 2, wherein the method comprises measuring the level of expression of at least five biomarkers from Tables 1-3.

4. The method of claim 3, wherein at least 2 of the measured biomarkers has an absolute value of the signature weight of greater than 0.025.

5. The method of claim 3, wherein the method comprises measuring the level of expression of at least 10 biomarkers from Tables 1-3 and wherein at least 5 of the measured biomarkers has an absolute value of the signature weight of greater than 0.025.

6. The method of claim 5, wherein the method comprises measuring the level of expression of at least 10 biomarkers from Tables 1-3 and wherein at least 5 of the measured biomarkers has an absolute value of the signature weight of greater than 0.03.

7. The method of any one of claims 4-6, wherein at least 20, 30, 40, or 50 biomarkers are measured.

8. The method of any one of claims 1-6, wherein the expression level of the measured biomarkers are determined to be not significantly different as compared to a control level of expression, wherein the control level of expression comprises the level of expression of the biomarkers in a small cell neuroendocrine cancer or small-round-blue cell tumor.

9. The method of any one of claims 1-8, wherein the one or more biomarkers has an absolute value of the signature weight of greater than 0.025.

10. The method of any one of claims 1-9, wherein 1, 2, 3, 4, 5, 6, 7, or 8 biomarkers from Tables 1-3 are excluded from being measured for expression levels in the biological sample from the cancer patient.

11. The method of any one of claims 1-10, further comprising comparing the level of expression to the level of expression of the biomarker(s) in a control sample.

12. The method of claim 11, wherein the control sample is a biological sample from a patient having a small cell neuroendocrine cancer or small-round-blue cell tumor.

13. The method of any one of claims 1-10, further comprising comparing the level of expression to a control level of expression of the biomarker(s).

14. The method of claim 13, wherein the control level of expression represents the level of expression of the biomarker in a small cell neuroendocrine cancer or small-round-blue cell tumor.

15. The method of any one of claims 1-14, wherein the biological sample was a tissue sample, a blood sample, a biopsy sample, a saliva sample, or a tumor sample.

16. The method of any one of claims 1-15, wherein the biological sample comprises tumor tissue.

17. The method of any one of claims 1-16, wherein the biological sample comprises metastatic tumor tissue or is from the lymph nodes.

18. The method of any one of claims 1-17, wherein the subject has been treated for a cancer.

19. The method of claim 18, wherein the treatment comprises a targeted therapy.

20. The method of any one of claims 1-19, further comprising evaluating tumor size and/or lymph node status.

21. The method of one any of claims 1-20, further comprising calculating a risk score for the patient.

22. The method of claim 21, wherein the risk score indicates a risk of decreased overall survival, metastasis, and/or recurrence.

23. The method of any one of claims 1-22, further comprising treating the patient for a small cell neuroendocrine cancer or small-round-blue cell tumor (SRBCT).

24. The method of claim 23, wherein the treatment comprises ABT-263, NSC-207895, NPK76-II-72-1, XMD13-2, MP470, BX-912, GW-2580, GSK1070916, WZ3105, GSK690693, OSI-027, FK866, I-BET-762, GSK429286A, UNC0638, PHA-793887, QL-X-138, Vorinostat, Tubastatin A, CX-5461, or combinations thereof.

25. The method of claim 23, wherein the treatment comprises vorinostat, VU0238429, CUDC-101, BX-912, oxaliplatin, alisertib, salinomycin, BMS-754807, KX2-391, D-64131, oxyquinoline, axitinib, barasertib, BI-78D3, M-344, evodiamine, UNBS-5162, vinorelbine, albendazole, BI-2536, or combinations thereof.

26. The method of claim 23, wherein the treatment comprises a drug listed in Table 4A, 4B, or combinations thereof.

27. A method for treating small cell neuroendocrine (SCN) cancer or small-round-blue cell tumor (SRBCT) in a patient comprising administering a cancer treatment to the patient, wherein the cancer treatment is a treatment selected from Table 4A, 4B, or combinations of treatments from Table 4A and/or 4B.

28. The method of claim 27, wherein the patient has been determined to have a SCN or SRBCT cancer based on the level of expression of one or more biomarkers from tables 1-3 in a biological sample from the patient.

29. A method for treating SCN or SRBCT cancer in a patient comprising administering a cancer treatment to a patient determined to have a SCN or SRBCT cancer, wherein the patient was determined to have a SCN or SRBCT by measuring the expression level of one or more biomarkers from tables 1-3 in a biological sample from the patient.

30. The method of any one of claims 27-29, wherein the patient has an epithelial or hematological cancer.

31. The method of any one of claims 28-30, wherein the method comprises measuring the level of expression of at least five biomarkers from Tables 1-3.

32. The method of any one of claims 28-31, wherein the expression level of the measured biomarkers were determined to be not significantly different than a control level of expression, wherein the control level of expression comprises the level of expression of the biomarkers in a SCN or SRBCT cancer.

33. The method of any one of claims 28-32, wherein the one or more biomarkers has an absolute value of the signature weight of greater than 0.025.

34. The method of any one of claims 28-33, wherein 1, 2, 3, 4, 5, 6, 7, or 8 biomarkers from Tables 1-3 are excluded from being measured for expression levels in the biological sample from the cancer patient.

35. The method of any one of claims 27-34, wherein the biological sample from the patient comprises a tissue sample, a blood sample, a biopsy sample, a saliva sample, or a tumor sample.

36. The method of any one of claims 27-35, wherein the biological sample comprises tumor tissue.

37. The method of any one of claims 27-36, wherein the biological sample comprises metastatic tumor tissue or is from the lymph nodes.

38. The method of any one of claims 27-37, wherein the subject has been treated for a cancer.

39. The method of claim 38, wherein the treatment comprises a targeted therapy.

40. The method of any one of claims 27-39, wherein the treatment comprises ABT-263, NSC-207895, NPK76-II-72-1, XMD13-2, MP470, BX-912, GW-2580, GSK1070916, WZ3105, GSK690693, OSI-027, FK866, I-BET-762, GSK429286A, UNC0638, PHA-793887, QL-X-138, Vorinostat, Tubastatin A, CX-5461, or combinations thereof.

41. The method of any one of claims 27-39, wherein the treatment comprises vorinostat, VU0238429, CUDC-101, BX-912, oxaliplatin, alisertib, salinomycin, BMS-754807, KX2-391, D-64131, oxyquinoline, axitinib, barasertib, BI-78D3, M-344, evodiamine, UNBS-5162, vinorelbine, albendazole, BI-2536, or combinations thereof.

42. The method of any one of claims 27-39, wherein the treatment comprises a drug listed in Table 4A, 4B, or combinations thereof.

43. A method for prognosing a cancer patient or for diagnosing a SCN or SRBCT cancer, comprising:
measuring the expression level of one or more biomarkers from Tables 1-3 in a biological sample from the patient;
comparing the expression level of the at least one biomarker to a control level of expression, wherein the control level of expression comprises the level of expression of the biomarkers in a SCN or SRBCT cancer; and
diagnosing the patient as having a SCN or SRBCT cancer when the level of expression of the measured biomarker is not substantially different from the control level of expression.

44. The method of claim 43, wherein the patient has an epithelial or hematological cancer.

45. The method of claim 43 or 44, wherein the method comprises measuring the level of expression of at least five biomarkers from Tables 1-3.

46. The method of any one of claims 43-45, wherein 1, 2, 3, 4, 5, 6, 7, or 8 biomarkers from Tables 1-3 are excluded from being measured for expression levels in the biological sample from the cancer patient.

47. The method of any one of claims 43-46, wherein the one or more biomarkers has an absolute value of the signature weight of greater than 0.025.

48. The method of any one of claims 43-47, further comprising comparing the level of expression to the level of expression of the biomarker(s) in a control sample.

49. The method of any one of claims 43-48, wherein the biological sample was a tissue sample, a blood sample, a biopsy sample, a saliva sample, or a tumor sample.

50. The method of any one of claims 43-49, wherein the biological sample comprises tumor tissue.

51. The method of any one of claims 43-50, wherein the biological sample comprises metastatic tumor tissue or is from the lymph nodes.

52. The method of any one of claims 43-51, wherein the subject has been treated for a cancer.

53. The method of claim 52, wherein the treatment comprises a targeted therapy.

54. The method of any one of claims 43-53, further comprising evaluating tumor size and/or lymph node status.

55. The method of one any of claims 43-54, further comprising calculating a risk score for the patient.

56. The method of claim 55, wherein the risk score indicates a risk of decreased overall survival, metastasis, and/or recurrence.

57. The method of any one of claims 43-56, further comprising treating the patient for a small cell neuroendocrine cancer.

58. The method of claim 57, wherein the treatment comprises ABT-263, NSC-207895, NPK76-II-72-1, XMD13-2, MP470, BX-912, GW-2580, GSK1070916, WZ3105, GSK690693, OSI-027, FK866, I-BET-762, GSK429286A, UNC0638, PHA-793887, QL-X-138, Vorinostat, Tubastatin A, CX-5461, or combinations thereof.

59. The method of claim 57, wherein the treatment comprises vorinostat, VU0238429, CUDC-101, BX-912, oxaliplatin, alisertib, salinomycin, BMS-754807, KX2-391, D-64131, oxyquinoline, axitinib, barasertib, BI-78D3, M-344, evodiamine, UNBS-5162, vinorelbine, albendazole, BI-2536, or combinations thereof.

60. The method of claim 57, wherein the treatment comprises a drug listed in Table 4A, 4B, or combinations thereof.

61. A method for treating small cell neuroendocrine cancer in a patient comprising administering a cancer treatment to a patient determined to have a small cell neuroendocrine cancer, wherein the patient was determined to have a small cell neuroendocrine cancer by measuring the expression level of at least ten biomarkers from tables 1-3 in a biological sample from the patient; wherein the biomarker has an absolute value of the signature weight of greater than 0.025; and wherein the treatment comprises ABT-263, NSC-207895, NPK76-II-72-1, XMD13-2, MP470, BX-912, GW-2580, GSK1070916, WZ3105, GSK690693, OSI-027, FK866, I-BET-762, GSK429286A, UNC0638, PHA-793887, QL-X-138, Vorinostat, Tubastatin A, CX-5461, or combinations thereof.

62. A method for treating small cell neuroendocrine cancer in a patient comprising administering a cancer treatment to a patient determined to have a small cell neuroendocrine cancer, wherein the patient was determined to have a small cell neuroendocrine cancer by measuring the expression level of at least ten biomarkers from tables 1-3 in a biological sample from the patient; wherein the biomarker has an absolute value of the signature weight of greater than 0.025; and wherein the treatment comprises vorinostat, VU0238429, CUDC-101, BX-912, oxaliplatin, alisertib, salinomycin, BMS-754807, KX2-391, D-64131, oxyquinoline, axitinib, barasertib, BI-78D3, M-344, evodiamine, UNBS-5162, vinorelbine, albendazole, BI-2536, or combinations thereof.

63. The method of claim 61 or 62, wherein at least one biomarker has an absolute value of the signature weight of greater than 0.04.

64. The method of claim 63, wherein at least 2 biomarkers have an absolute value of the signature weight of greater than 0.03.
